(19) United States
(12) Patent Application Publication
(10) Pub. No.: US 2023/0203003 A1
(43) Pub. Date: Jun. 29, 2023

(54) Notch Inhibitors and Uses Thereof

(71) Applicant: StemSynergy Therapeutics, Inc., Miami, FL (US)

(72) Inventors: Darren Orton, Nashville, TN (US); William Guerrant, Davie, FL (US); Matthew Knowe, Old Hickory, TN (US); Dennis Liang Fei, Miami, FL (US)

(21) Appl. No.: 17/925,917

(22) PCT Filed: May 21, 2021
(86) PCT No.: PCT/US2021/033696

§ 371 Date: Nov. 17, 2022

Related U.S. Application Data
(60) Provisional application No. 63/028,194, filed on May 21, 2020.

(51) Int. Cl. C07D 401/12; C07D 413/12; C07D 405/12; C07D 471/04; C07D 405/14; C07D 215/22; A61K 45/06

(52) U.S. Cl. CPC C07D 401/12; A61K 45/06; C07D 215/22; C07D 405/12; C07D 405/14; C07D 413/12; C07D 471/04

(57) ABSTRACT

Disclosed herein, inter alia, are compounds for inhibiting Notch and uses thereof.

### CROSS-REFERENCES TO RELATED APPLICATIONS

This application claims the benefit of U.S. Provisional Application No. 63/028,194, filed May 21, 2020, which is incorporated herein by reference in its entirety and for all purposes.

### BACKGROUND

Notch is a major developmental pathway that regulates cancer stem cells (CSCs) in Notch-driven cancers. Notch signaling is initiated upon the physical interaction of cells expressing ligands with neighboring cells expressing Notch receptors. Notch ligand/receptor interaction results in irreversible cleavage of Notch receptors by gamma-secretase and subsequent generation of Notch intracellular domains (NICDs). NICDs translocate to the nucleus and are required for the stepwise formation of an active Notch Transcription Complex (NTC) that includes recruitment of the DNA-binding protein CSL, followed by the transcriptional coactivator Mastermind-like 1. The NTC subsequently recruits additional coactivators and drives transcription of target genes. Compounds and methods that prevent NTC assembly will inhibit NICDs-directed transcription, thus reducing the growth of Notch associated cancers. Disclosed herein, inter alia, are solutions to these and other problems known in the art.

### BRIEF SUMMARY

In an aspect is provided a compound having the formula:

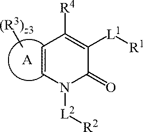

or a salt (e.g., pharmaceutically acceptable salt) thereof.

L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene (e.g., C₁—C₈, C₁—C₆, or C₁—C₄) or substituted or unsubstituted heteroalkylene (e.g., 2 to 8 membered, 2 to 6 membered, or 2 to 4 membered).

R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene (e.g., C₁—C₈, C₁—C₆, or C₁—C₄) or substituted or unsubstituted heteroalkylene (e.g., 2 to 8 membered, 2 to 6 membered, or 2 to 4 membered).

R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered),

Ring A is C₅—C₆ cycloalkyl, 5 to 6 membered heterocycloalkyl, phenyl, or 5 to 6 membered heteroaryl.

R³ is independently halogen, oxo, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)m3, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); two adjacent R³ substituents may optionally be joined to form a substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

z3 is independently an integer from 0 to 8.

R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or -OR^{4D}.

R^{1A}, R^{1B}, R^{1C},R^{1D}, R^{2A}, R^{2B}, R^{2C}, R^{2D}, R^{3A}, R^{3B}, R^{3C}, R^{3D}, R^{4A}, R^{4B}, R^{4D}, R^{L1}, and R^{L2} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, -COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered) ; R^{2A} and R^{2B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered) ; R^{3A} and R^{3B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered) ; R^{4A} and R^{4B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

X¹, X², X³, and X⁴, are independently —F, —Cl, —Br, or —I.

n1, n2, and n3 are independently an integer from 0 to 4.

m1, m2, m3, v1, v2, and v3 are independently 1 or 2; wherein the compound is not:

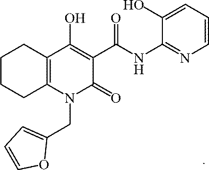

In an aspect is provided a pharmaceutical composition including a compound described herein, or a salt (e.g., pharmaceutically acceptable salt) thereof, and a pharmaceutically acceptable excipient.

In an aspect is provided a method of decreasing the level of Notch (e.g., one or more of Notch 1, Notch 2, Notch 3, and/or Notch 4) protein activity in a subject, the method including administering a compound described herein, or a salt (e.g., pharmaceutically acceptable salt) thereof, to the subject.

In an aspect is provided a method of decreasing the level of Notch (e.g., one or more of Notch 1, Notch 2, Notch 3, and/or Notch 4) activity in a cell, the method including contacting the cell with a compound described herein, or a salt (e.g., pharmaceutically acceptable salt) thereof.

In an aspect is provided a method of decreasing the level of CSL-Notch (e.g., one or more of Notch 1, Notch 2, Notch 3, and/or Notch 4)-Mastermind complex activity in a subject, the method including administering a compound described herein, or a salt (e.g., pharmaceutically acceptable salt) thereof, to the subject.

In an aspect is provided a method of decreasing the level of CSL-Notch (e.g., one or more of Notch 1, Notch 2, Notch 3, and/or Notch 4)-Mastermind complex activity in a cell, the method including contacting the cell with a compound described herein, or a salt (e.g., pharmaceutically acceptable salt) thereof.

In an aspect is provided a method of inhibiting cancer growth in a subject in need thereof, the method including administering to the subject in need thereof an effective amount of a compound described herein, or a salt (e.g., pharmaceutically acceptable salt) thereof.

In an aspect is provided a method of treating a cancer in a subject in need thereof, the method including administering to the subject in need thereof an effective amount of a compound described herein, or a salt (e.g., pharmaceutically acceptable salt) thereof.

### DETAILED DESCRIPTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-and multivalent radicals. The alkyl may include a designated number of carbons (e.g., C₁—C₁₀ means one to ten carbons). Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds. In embodiments, the alkyl is fully saturated. In embodiments, the alkyl is monounsaturated. In embodiments, the alkyl is polyunsaturated.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. In embodiments, an alkenylene includes one or more double bonds. In embodiments, an alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as -NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. In embodiments, a heteroalkenylene includes one or more double bonds. In embodiments, a heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems are bridged monocyclic rings or fused bicyclic rings. In embodiments, bridged monocyclic rings contain a monocyclic cycloalkyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, andbicyclo[4.2.1]nonane. In embodiments, fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. In embodiments, cycloalkyl groups are optionally substituted with one or two groups which are independently oxo or thia. In embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic cycloalkyl is attached to the parent molecular moiety through any carbon atom contained within the base ring. In embodiments, multicyclic cycloalkyl ring systems are a monocyclic cycloalkyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic cycloalkyl groups include, but are not limited to tetradecahydrophenanthrenyl, perhydrophenothiazin-1-yl, and perhydrophenoxazin-1-yl.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. In embodiments, monocyclic cycloalkenyl ring systems are cyclic hydrocarbon groups containing from 3 to 8 carbon atoms, where such groups are unsaturated (i.e., containing at least one annular carbon carbon double bond), but not aromatic. Examples of monocyclic cycloalkenyl ring systems include cyclopentenyl and cyclohexenyl. In embodiments, bicyclic cycloalkenyl rings are bridged monocyclic rings or a fused bicyclic rings. In embodiments, bridged monocyclic rings contain a monocyclic cycloalkenyl ring where two non adjacent carbon atoms of the monocyclic ring are linked by an alkylene bridge of between one and three additional carbon atoms (i.e., a bridging group of the form (CH₂)_{w}, where w is 1, 2, or 3). Representative examples of bicyclic cycloalkenyls include, but are not limited to, norbornenyl and bicyclo[2.2.2]oct 2 enyl. In embodiments, fused bicyclic cycloalkenyl ring systems contain a monocyclic cycloalkenyl ring fused to either a phenyl, a monocyclic cycloalkyl, a monocyclic cycloalkenyl, a monocyclic heterocyclyl, or a monocyclic heteroaryl. The bridged or fused bicyclic cycloalkenyl is attached to the parent molecular moiety through any carbon atom contained within the monocyclic cycloalkenyl ring. In embodiments, cycloalkenyl groups are optionally substituted with one or two groups which are independently oxo or thia. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic cycloalkenyl is attached to the parent molecular moiety through any carbon atom contained within the base ring. In embodiments, multicyclic cycloalkenyl rings contain a monocyclic cycloalkenyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two ring systems independently selected from the group consisting of a phenyl, a monocyclic heteroaryl, a monocyclic cycloalkyl, a monocyclic cycloalkenyl, and a monocyclic heterocyclyl.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through an atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through an atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through an atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁—C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Nonlimiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl, benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ 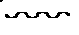 ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁—C₄ alkylsulfonyl”).

The term “alkylarylene” as an arylene moiety covalently bonded to an alkylene moiety (also referred to herein as an alkylene linker). In embodiments, the alkylarylene group has the formula:

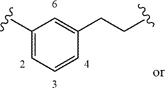

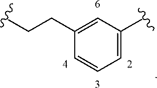

An alkylarylene moiety may be substituted (e.g., with a substituent group) on the alkylene moiety or the arylene linker (e.g. at carbons 2, 3, 4, or 6) with halogen, oxo, —N₃, —CF₃, —CCl₃, —CBr₃, —CI₃, —CN, —CHO, —OH, —NH₂, —COOH, —CONH₂, —NO₂, —SH, —SO₂CH₃, —SO₃H, —OSO₃H, —SO₂NH₂, —NHNH₂, —ONH₂, —NHC(O)NHNH₂, substituted or unsubstituted C₁-C₅ alkyl or substituted or unsubstituted 2 to 5 membered heteroalkyl). In embodiments, the alkylarylene is unsubstituted.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′”, —OC(O)R′, —C(O)R′, —CO₂R′, —CONR′R″, —OC(O)NR′R″, —NR″C(O)R′, —NR′—C(O)NR″R′”, —NR″C(O)₂R′, —NR—C(NR′R″R′”)═NR″, —NR—C(NR′R″)═NR′”, —S(O)R′, —S(O)₂R′, —S(O)₂NR′R″, —NRSO₂R′, —NR′NR″R″’, —ONR′R″, —NR′C(O)NR″NR″‘R⁗, —CN, —NO₂, —NR′SO₂R″, —NR′C(O)R″, —NR′C(O)—OR″, —NR′OR″, in a number ranging from zero to (2 m′+1), where m′ is the total number of carbon atoms in such radical. R, R′, R″, R‴, and R⁗ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R‴, and R⁗ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′”, —OC(O)R′, —C(O)R′, —CO₂R′, —CONR′R″, —OC(O)NR′R″, —NR″C(O)R′, —NR′—C(O)NR″R″’, —NR″C(O)₂R′, —NR—C(NR′R″R′”)═NR″, —NR—C(NR′R″)═NR′”, —S(O)R′, —S(O)₂R′, —S(O)₂NR′R″, —NRSO₂R′, —NR′NR″R″’, —ONR′R″, —NR′C(O)NR″NR″‘R⁗, —CN, —NO₂, —R′, —N₃, —CH(Ph)₂, fluoro(C₁—C₄)alkoxy, and fluoro(C₁—C₄)alkyl, —NR′SO₂R″, —NR′C(O)R″, —NR′C(O)—OR″, —NR′OR″, in a number ranging from zero to the total number of open valences on the aromatic ring system; and where R′, R″, R‴, and R⁗ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R‴, and R⁗ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)-(CRR′)_{q}-U-, wherein T and U are independently -NR-, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′”)_{d}-, where s and d are independently integers of from 0 to 3, and X′ is —O—, —NR′—, —S—, —S(O)—, —S(O)₂—, or —S(O)₂NR′—. The substituents R, R′, R″, and R‴ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group” or “substituent” as used herein, means a group selected from the following moieties:
(A) oxo, halogen, —CCl₃, —CBr₃, —CF₃, —CI₃, CHCl₂, —CHBr₂, —CHF₂, —CHI2, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁—C₈ alkyl, C₁—C₆ alkyl, or C₁—C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃—C₈ cycloalkyl, C₃—C₆ cycloalkyl, or C₅—C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆—C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁—C₂₀ alkyl, C₁—C₁₂ alkyl, C₁—C₈ alkyl, C₁—C₆ alkyl, C₁—C₄ alkyl, or C₁—C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃—C₁₀ cycloalkyl, C₃—C₈ cycloalkyl, C₃—C₆ cycloalkyl, C₄—C₆ cycloalkyl, or C₅—C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆—C₁₂ aryl, C₆—C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, CHCl₂, —CHBr₂, —CHF₂, —CHI2, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁—C₈ alkyl, C₁—C₆ alkyl, or C₁—C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃—C₈ cycloalkyl, C₃—C₆ cycloalkyl, or C₅—C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆—C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁—C₂₀alkyl, C₁—C₁₂ alkyl, C₁—C₈ alkyl, C₁—C₆alkyl, C₁—C₄ alkyl, or C₁—C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃—C₁₀ cycloalkyl, C₃—C₈ cycloalkyl, C₃—C₆ cycloalkyl, C₄—C₆ cycloalkyl, or C₅—C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆—C₁₂ aryl, C₆—C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁—C₈ alkyl, C₁—C₆ alkyl, or C₁—C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃—C₈ cycloalkyl, C₃—C₆ cycloalkyl, or C₅—C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆—C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁—C₂₀ alkyl, C₁—C₁₂ alkyl, C₁—C₈ alkyl, C₁—C₆ alkyl, C₁—C₄ alkyl, or C₁—C₂ alkyl), heteroalkyl (e.g., 2 to 20 membered heteroalkyl, 2 to 12 membered heteroalkyl, 2 to 8 membered heteroalkyl, 2 to 6 membered heteroalkyl, 4 to 6 membered heteroalkyl, 2 to 3 membered heteroalkyl, or 4 to 5 membered heteroalkyl), cycloalkyl (e.g., C₃—C₁₀ cycloalkyl, C₃—C₈ cycloalkyl, C₃—C₆ cycloalkyl, C₄—C₆ cycloalkyl, or C₅—C₆ cycloalkyl), heterocycloalkyl (e.g., 3 to 10 membered heterocycloalkyl, 3 to 8 membered heterocycloalkyl, 3 to 6 membered heterocycloalkyl, 4 to 6 membered heterocycloalkyl, 4 to 5 membered heterocycloalkyl, or 5 to 6 membered heterocycloalkyl), aryl (e.g., C₆—C₁₂ aryl, C₆—C₁₀ aryl, or phenyl), or heteroaryl (e.g., 5 to 12 membered heteroaryl, 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —N₃, unsubstituted alkyl (e.g., C₁—C₈ alkyl, C₁—C₆ alkyl, or C₁—C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃—C₈ cycloalkyl, C₃—C₆ cycloalkyl, or C₅—C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆—C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁—C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃—C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆—C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁—C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and each substituted or unsubstituted heteroaryl is a substituted or unsubstituted 5 to 6 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁—C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃—C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆—C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁—C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃—C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆—C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁—C₈ alkyl, each substituted or unsubstituted heteroalkyl is a substituted or unsubstituted 2 to 8 membered heteroalkyl, each substituted or unsubstituted cycloalkyl is a substituted or unsubstituted C₃—C₇ cycloalkyl, each substituted or unsubstituted heterocycloalkyl is a substituted or unsubstituted 3 to 7 membered heterocycloalkyl, each substituted or unsubstituted aryl is a substituted or unsubstituted phenyl, and/or each substituted or unsubstituted heteroaryl is a substituted or unsubstituted 5 to 6 membered heteroaryl. In some embodiments, each substituted or unsubstituted alkylene is a substituted or unsubstituted C₁—C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted phenylene, and/or each substituted or unsubstituted heteroarylene is a substituted or unsubstituted 5 to 6 membered heteroarylene. In some embodiments, the compound is a chemical species set forth herein, for example in the Examples section, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

In a recited claim or chemical formula description herein, each R substituent or L linker that is described as being “substituted” without reference as to the identity of any chemical moiety that composes the “substituted” group (also referred to herein as an “open substitution” on a R substituent or L linker or an “openly substituted” R substituent or L linker), the recited R substituent or L linker may, in embodiments, be substituted with one or more first substituent groups as defined below.

The first substituent group is denoted with a corresponding first decimal point numbering system such that, for example, R¹ may be substituted with one or more first substituent groups denoted by R^{1.1}, R² may be substituted with one or more first substituent groups denoted by R^{2.1}, R³ may be substituted with one or more first substituent groups denoted by R^{3.1}, R⁴ may be substituted with one or more first substituent groups denoted by R^{4.1}, R⁵ may be substituted with one or more first substituent groups denoted by R^{5.1}, and the like up to or exceeding an R¹⁰⁰ that may be substituted with one or more first substituent groups denoted by R^{100.1}. As a further example, R^{1A} may be substituted with one or more first substituent groups denoted by R^{1A.1}, R^{2A} may be substituted with one or more first substituent groups denoted by R^{2A.1}, R^{3A} may be substituted with one or more first substituent groups denoted by R^{3A.1}, R^{4A} may be substituted with one or more first substituent groups denoted by R^{4A.1}, R^{5A} may be substituted with one or more first substituent groups denoted by R^{5A.1} and the like up to or exceeding an R^{100A} may be substituted with one or more first substituent groups denoted by R^{100A.1} . As a further example, L¹ may be substituted with one or more first substituent groups denoted by R^{L1.1}, L² may be substituted with one or more first substituent groups denoted by R^{L2.1}, L³ may be substituted with one or more first substituent groups denoted by R^{L3.1}, L⁴ may be substituted with one or more first substituent groups denoted by R^{L4.1}, L⁵ may be substituted with one or more first substituent groups denoted by R^{L5.1} and the like up to or exceeding an L¹⁰⁰ which may be substituted with one or more first substituent groups denoted by R^{L100.1}. Thus, each numbered R group or L group (alternatively referred to herein as R^{WW} or L^{WW} wherein “WW” represents the stated superscript number of the subject R group or L group) described herein may be substituted with one or more first substituent groups referred to herein generally as R^{WW.1} or R^{LWW.1}, respectively. In turn, each first substituent group (e.g. R^{1.1}, R^{2.1}, R^{3.1}, R^{4.1}, R^{5.1}... R^{100.1}; R^{1A.1}, R^{2A.1}, R^{3A.1}, R^{4A.1}, R^{5A.1} ... R^{100A.1}; R^{L1.1}, R^{L2.1}, R^{L3.1}, R^{L4.1}, R^{L5.1} ... R^{L100.1}) may be further substituted with one or more second substituent groups (e.g. R^{1.2}, R^{2.2}, R^{3.2}, R^{4.2}, R^{5.2}... R^{100.2}; R^{1A.2}, R^{2A.2}, R^{3A.2}, R^{4A.2}, R^{5A.2} ... R^{100A.2.}; R^{L1.2}, R^{L2.2}, R^{L3.2}, R^{L4.2}, R^{L5.2}... R^{L100.2}, respectively). Thus, each first substituent group, which may alternatively be represented herein as R^{WW.1} as described above, may be further substituted with one or more second substituent groups, which may alternatively be represented herein as R^{WW.2}.

Finally, each second substituent group (e.g. R^{1.2}, R^{2.2}, R^{3.2}, R^{4.2}, R^{5.2}... R^{100.2}; R^{1A.2}, R^{2A.2}, R^{3A.2}, R^{4A.2}, R^{5A.2} ... R^{100A.2}; R^{L1.2}, R^{L2.2}, R^{L3.2}, R^{L4.2}, R^{L5.2} ... R^{L100.2}) may be further substituted with one or more third substituent groups (e.g. R^{1.3}, R^{2.3}, R^{3.3}, R^{4.3}, R^{5.3}... R^{100.3}; R^{1A.3}, R^{2A.3}, R^{3A.3}, R^{4A.3}, R^{5A.3} ... R^{100A.3.}; R^{L1.3}, R^{L2.3}, R^{L3.3}, R^{L4.3}, R^{L5.3} ... R^{L100.3}; respectively). Thus, each second substituent group, which may alternatively be represented herein as R^{WW.2} as described above, may be further substituted with one or more third substituent groups, which may alternatively be represented herein as R^{WW.3}. Each of the first substituent groups may be optionally different. Each of the second substituent groups may be optionally different. Each of the third substituent groups may be optionally different.

Thus, as used herein, R^{WW} represents a substituent recited in a claim or chemical formula description herein which is openly substituted. “WW” represents the stated superscript number of the subject R group (1, 2, 3, 1A, 2A, 3A, 1B, 2B, 3B, etc.). Likewise, L^{WW} is a linker recited in a claim or chemical formula description herein which is openly substituted. Again, “WW” represents the stated superscript number of the subject L group (1, 2, 3, 1A, 2A, 3A, 1B, 2B, 3B, etc.). As stated above, in embodiments, each R^{WW} may be unsubstituted or independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group may be unsubstituted or independently substituted with one or more third substituent groups, referred to herein as R^{WW.3}. Similarly, each L^{WW} linker may be unsubstituted or independently substituted with one or more first substituent groups, referred to herein as R^{LWW.1}; each first substituent group, R^{LWW.1}, may be unsubstituted or independently substituted with one or more second substituent groups, referred to herein as R^{LWW.2}; and each second substituent group may be unsubstituted or independently substituted with one or more third substituent groups, referred to herein as R^{LWW.3}. Each first substituent group is optionally different. Each second substituent group is optionally different. Each third substituent group is optionally different. For example, if R^{WW} is phenyl, the said phenyl group is optionally substituted by one or more R^{WW.1} groups as defined herein below, e.g. when R^{WW.1} is R^{WW.2} substituted alkyl, examples of groups so formed include but are not limited to itself optionally substituted by 1 or more R^{WW.2},which R^{WW.2} is optionally substituted by one or more R^{WW.3}. By way of example when R^{WW.1} is alkyl, groups that could be formed, include but are not limited to:

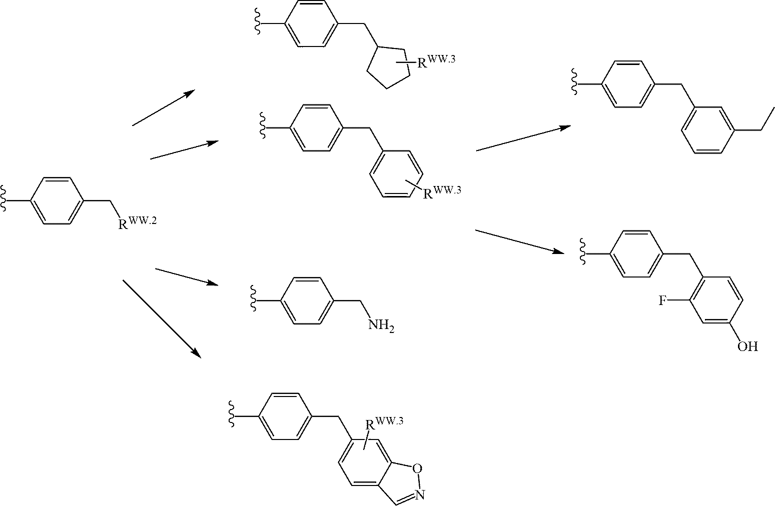

R^{WW.1} is independently oxo, halogen, —CX^{WW.1}3, —CHX^{WW.1}₂, —CH₂X^{WW.1}, —OCX^{WW.1}₃, —OCH₂X^{WW.1}, —OCHX^{WW.1}₂, —CN, —OH, —NH2, —COOH, —CONH2, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.2}-substituted or unsubstituted alkyl (e.g., C₁—C₈, C₁—C₆, C₁—C₄, or C₁—C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW}^{.}²-substituted or unsubstituted cycloalkyl (e.g., C₃—C₈, C₃—C₆, C₄—C₆, or C₅—C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆—C₁₂, C₆—C₁₀, or phenyl), or R^{WW}^{.}²-substituted or unsubstituted heteroaryl (e.g., 5 to 12 membered, 5 to 10 membered, 5 to 9 membered, or 5 to 6 membered). In embodiments, R^{WW.1} is independently oxo, halogen, —CX^{WW.1}₃, —CHX^{WW.1}₂, —CH₂X^{WW.1}, —OCX^{WW.1}₃, —OCH₂X^{WW.1}, —OCHX^{WW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁—C₈, C₁—C₆, C₁—C₄, or C₁—C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃—C₈, C₃—C₆, C₄—C₆, or C₅—C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆—C₁₂, C₆—C₁₀, or phenyl), or unsubstituted heteroaryl (e.g., 5 to 12 membered, 5 to 10 membered, 5 to 9 membered, or 5 to 6 membered). X^{WW.1} is independently —F, —Cl, —Br, or —I.

R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.3}-substituted or unsubstituted alkyl (e.g., C₁—C₈, C₁—C₆, C₁—C₄, or C₁—C₂), R^{WW.2}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃—C₈, C₃—C₆, C₄—C₆, or C₅-C₆), R^{WW.2}-substituted or unsubstituted heterocycloalkyl (e.g., 3 to 8 membered, 3 to 6 membered, 4 to 6 membered, 4 to 5 membered, or 5 to 6 membered), R^{WW.2}-substituted or unsubstituted aryl (e.g., C₆—C₁₂, C₆—C₁₀, or phenyl), or R^{WW.2}-substituted or unsubstituted heteroaryl (e.g., 5 to 12 membered, 5 to 10 membered, 5 to 9 membered, or 5 to 6 membered). In embodiments, R^{WW.2} is independently oxo, halogen, —CX^{WW.2}₃, —CHX^{WW.2}₂, —CH₂X^{WW.2}, —OCX^{WW.2}₃, —OCH₂X^{WW.2}, —OCHX^{WW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═ (O)H, —NHC(O)—OH, —NHOH, —N₃, unsubstituted alkyl (e.g., C₁—C₈, C₁—C₆, C₁—C₄, or C₁—C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃—C₈, C₃—C₆, C₄—C₆, or C₅—C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆—C₁₂, C₆—C₁₀, or phenyl), or unsubstituted heteroaryl (e.g., 5 to 12 membered, 5 to 10 membered, 5 to 9 membered, or 5 to 6 membered). X^{WW.2} is independently —F, —Cl, —Br, or —I.

R^{WW.2} is independently oxo, halogen, —CX^{WW.3}₃, —CHX^{WW.3}₂, —CH₂X^{WW.3}, —OCX^{WW.3}₃, —OCH₂X^{WW.3}, —OCHX^{WW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁—C₈, C₁—C₆, C₁—C₄, or C₁—C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃—C₈, C₃—C₆, C₄—C₆, or C₅—C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆—C₁₂, C₆—C₁₀, or phenyl), or unsubstituted heteroaryl (e.g., 5 to 12 membered, 5 to 10 membered, 5 to 9 membered, or 5 to 6 membered). X^{WW.3} is independently —F, —Cl, —Br, or —I.

Where two different R^{WW} substituents are joined together to form an openly substituted ring (e.g. substituted cycloalkyl, substituted heterocycloalkyl, substituted aryl or substituted heteroaryl), in embodiments the openly substituted ring may be independently substituted with one or more first substituent groups, referred to herein as R^{WW.1}; each first substituent group, R^{WW.1}, may be unsubstituted or independently substituted with one or more second substituent groups, referred to herein as R^{WW.2}; and each second substituent group, R^{WW.2}, may be unsubstituted or independently substituted with one or more third substituent groups, referred to herein as R^{WW.2}; and each third substituent group, R^{WW.2}, is unsubstituted. Each first substituent group is optionally different. Each second substituent group is optionally different. Each third substituent group is optionally different. In the context of two different R^{WW} substituents joined together to form an openly substituted ring, the “WW” symbol in the R^{WW.1}, R^{WW.2} and R^{WW.2} refers to the designated number of one of the two different R^{WW} substituents. For example, in embodiments where R^{100A} and R^{100B} are optionally joined together to form an openly substituted ring, R^{WW.1} is R^{100A.1}, R^{WW.2} is R^{100A.2}, and R^{WW.2} is R^{100A.3.} Alternatively, in embodiments where R^{100A} and R^{100B} are optionally joined together to form an openly substituted ring, R^{WW.1} is R^{100B.1}, R^{WW.2} is R^{100B.2}, and R^{WW.2} is R^{100B.3}. R^{WW.1}, R^{WW.2} and R^{WW.2} in this paragraph are as defined in the preceding paragraphs.

R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, -NHOH, —N₃, R^{LWW.2}-substituted or unsubstituted alkyl (e.g., C₁—C₈, C₁—C₆, C₁—C₄, or C₁—C₂), R^{LWW.2}-substituted or unsubstituted heteroalkyl (e.g., 2 to 8 membered, 2 to 6 membered, 4 to 6 membered, 2 to 3 membered, or 4 to 5 membered), R^{LWW.2}-substituted or unsubstituted cycloalkyl (e.g., C₃—C₈, C₃—C₆, C₄—C₆, or C₅-C₆), R^{LWW.2}-substituted or unsubstituted heterocycloalkyl (e.g., 3 to 8 membered, 3 to 6 membered, 4 to 6 membered, 4 to 5 membered, or 5 to 6 membered), R^{LWW.2}-substituted or unsubstituted aryl (e.g., C₆—C₁₂, C₆—C₁₀, or phenyl), or R^{LWW.2_}substituted or unsubstituted heteroaryl (e.g., 5 to 12 membered, 5 to 10 membered, 5 to 9 membered, or 5 to 6 membered). In embodiments, R^{LWW.1} is independently oxo, halogen, —CX^{LWW.1}₃, —CHX^{LWW.1}₂, —CH₂X^{LWW.1}, —OCX^{LWW.1}₃, —OCH₂X^{LWW.1}, —OCHX^{LWW.1}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁—C₈, C₁—C₆, C₁—C₄, or C₁—C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃—C₈, C₃—C₆, C₄—C₆, or C₅—C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆—C₁₂, C₆—C₁₀, or phenyl), or unsubstituted heteroaryl (e.g., 5 to 12 membered, 5 to 10 membered, 5 to 9 membered, or 5 to 6 membered). X^{LWW.1} is independently —F, —Cl, —Br, or —I.

R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{LWW.3}-substituted or unsubstituted alkyl (e.g., C₁—C₈, C₁—C₆, C₁—C₄, or C₁—C₂), R^{LWW.3}-substituted or unsubstituted heteroalkyl (e.g., 2 to 8 membered, 2 to 6 membered, 4 to 6 membered, 2 to 3 membered, or 4 to 5 membered), R^{WW.2}-substituted or unsubstituted cycloalkyl (e.g., C₃—C₈, C₃—C₆, C₄—C₆, or C₅—C₆), R^{LWW.3}-substituted or unsubstituted heterocycloalkyl (e.g., 3 to 8 membered, 3 to 6 membered, 4 to 6 membered, 4 to 5 membered, or 5 to 6 membered), R^{LWW.3}-substituted or unsubstituted aryl (e.g., C₆—C₁₂, C₆—C₁₀, or phenyl), or R^{LWW.3}-substituted or unsubstituted heteroaryl (e.g., 5 to 12 membered, 5 to 10 membered, 5 to 9 membered, or 5 to 6 membered). In embodiments, R^{LWW.2} is independently oxo, halogen, —CX^{LWW.2}₃, —CHX^{LWW.2}₂, —CH₂X^{LWW.2}, —OCX^{LWW.2}₃, —OCH₂X^{LWW.2}, —OCHX^{LWW.2}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁—C₈, C₁—C₆, C₁—C₄, or C₁—C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃—C₈, C₃—C₆, C₄—C₆, or C₅—C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆—C₁₂, C₆—C₁₀, or phenyl), or unsubstituted heteroaryl (e.g., 5 to 12 membered, 5 to 10 membered, 5 to 9 membered, or 5 to 6 membered). X^{LWW.2} is independently —F, —Cl, —Br, or —I.

R^{LWW.3} is independently oxo, halogen, —CX^{LWW.3}₃, —CHX^{LWW.3}₂, —CH₂X^{LWW.3}, —OCX^{LWW.3}₃, —OCH₂X^{LWW.3}, —OCHX^{LWW.3}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, unsubstituted alkyl (e.g., C₁—C₈, C₁—C₆, C₁—C₄, or C₁—C₂), unsubstituted heteroalkyl (e.g., 2 to 8 membered, 2 to 6 membered, 4 to 6 membered, 2 to 3 membered, or 4 to 5 membered), unsubstituted cycloalkyl (e.g., C₃—C₈, C₃-C₆, C₄—C₆, or C₅—C₆), unsubstituted heterocycloalkyl (e.g., 3 to 8 membered, 3 to 6 membered, 4 to 6 membered, 4 to 5 membered, or 5 to 6 membered), unsubstituted aryl (e.g., C₆—C₁₂, C₆-C₁₀, or phenyl), or unsubstituted heteroaryl (e.g., 5 to 12 membered, 5 to 10 membered, 5 to 9 membered, or 5 to 6 membered). X^{LWW.3} is independently —F, —Cl, —Br, or —I.

In the event that any R group recited in a claim or chemical formula description set forth herein (R^{WW} substituent) is not specifically defined in this disclosure, then that R group (R:^{WW} group) is hereby defined as independently oxo, halogen, —CX^{WW}₃, —CHX^{WW}₂, —CH₂X^{WW}, —OCX^{WW}₃, —OCH₂X^{WW}, —OCHX^{WW}₂, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)—OH, —NHOH, —N₃, R^{WW.1}-substituted or unsubstituted alkyl (e.g., C₁—C₈, C₁—C₆, C₁—C₄, or C₁—C₂), R^{WW.1}-substituted or unsubstituted heteroalkyl (e.g., 2 to 8 membered, 2 to 6 membered, 4 to 6 membered, 2 to 3 membered, or 4 to 5 membered), R^{WW.1}-substituted or unsubstituted cycloalkyl (e.g., C₃—C₈, C₃—C₆, C₄—C₆, or C₅-C₆), R^{WW.1}-substituted or unsubstituted heterocycloalkyl (e.g., 3 to 8 membered, 3 to 6 membered, 4 to 6 membered, 4 to 5 membered, or 5 to 6 membered), R^{WW.1}-substituted or unsubstituted aryl (e.g., C₆—C₁₂, C₆—C₁₀, or phenyl), or R^{WW.1}-substituted or unsubstituted heteroaryl (e.g., 5 to 12 membered, 5 to 10 membered, 5 to 9 membered, or 5 to 6 membered). X^{WW} is independently —F, —Cl, —Br, or —I. Again, “WW” represents the stated superscript number of the subject R group (e.g. 1, 2, 3, 1A, 2A, 3A, 1B, 2B, 3B, etc.). R^{WW.1}, R^{WW.2}, and R^{WW.2}, are as defined above.

In the event that any L linker group recited in a claim or chemical formula description set forth herein (i.e. an L^{WW} substituent) is not explicitly defined, then that L group (L^{WW} group) is herein defined as independently a bond, —O—, —NH—, —C(O)—, —C(O)NH—, —NHC(O)—, —NHC(O)NH—, —C(O)O—, —OC(O)—, —S—, —SO₂NH—, —NHSO₂—, R^{LWW.1}-substituted or unsubstituted alkylene (e.g., C₁—C₈, C₁—C₆, C₁—C₄, or C₁—C₂), R^{LWW.1}-substituted or unsubstituted heteroalkylene (e.g., 2 to 8 membered, 2 to 6 membered, 4 to 6 membered, 2 to 3 membered, or 4 to 5 membered), R^{LWW.1}-substituted or unsubstituted cycloalkylene (e.g., C₃—C₈, C₃—C₆, C₄—C₆, or C₅—C₆), R^{LWW.1}-substituted or unsubstituted heterocycloalkylene (e.g., 3 to 8 membered, 3 to 6 membered, 4 to 6 membered, 4 to 5 membered, or 5 to 6 membered), R^{LWW.1}-substituted or unsubstituted arylene (e.g., C₆—C₁₂, C₆—C₁₀, or phenyl), or R^{LWW.1}-substituted or unsubstituted heteroarylene (e.g., 5 to 12 membered, 5 to 10 membered, 5 to 9 membered, or 5 to 6 membered). Again, “WW” represents the stated superscript number of the subject L group (1, 2, 3, 1A, 2A, 3A, 1B, 2B, 3B, etc.). R^{LWW.1}, as well as R^{LWW.2} and R^{LWW.3}, are as defined above.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)-or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

As used herein, the term “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH₂, —C(O)OH, —N—hydroxysuccinimide, or -maleimide) and a second bioconjugate reactive group (e.g., sulfhydryl, sulfur-containing amino acid, amine, amine sidechain containing amino acid, or carboxylate) provided herein may be bound, for example, by covalent bond, linker (e.g., a first linker of second linker), or non-covalent bond (e.g. electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions, and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N—hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g., -sulfo-N-hydroxysuccinimide moiety) is covalently attached to the second bioconjugate reactive group (e.g., an amine).

Useful bioconjugate reactive moieties used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding;
(m) metal bonding to reactive phosphorus groups (e.g. phosphines) to form, for example, phosphate diester bonds;
(n) azides coupled to alkynes using copper catalyzed cycloaddition click chemistry; and
(o) biotin conjugate can react with avidin or streptavidin to form an avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

“Analog,” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished as R^{13.A}, R^{13.B}, R^{13.C}, R¹³^{.}^{D}, etc., wherein each of R^{13.A}, R¹³^{.}^{B}, R^{13.C}, R^{13.D}, etc. is defined within the scope of the definition of R¹³ and optionally differently.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (-)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer’s, normal sucrose, normal glucose, binders, fillers, disintegrants, lubricants, coatings, sweeteners, flavors, salt solutions (such as Ringer’s solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/- 10% of the specified value. In embodiments, about includes the specified value.

“Contacting” is used in accordance with its plain ordinary meaning and refers to the process of allowing at least two distinct species (e.g., chemical compounds including biomolecules or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

As defined herein, the term “activation”, “activate”, “activating”, “activator” and the like in reference to a protein-inhibitor interaction means positively affecting (e.g., increasing) the activity or function of the protein relative to the activity or function of the protein in the absence of the activator. In embodiments activation means positively affecting (e.g., increasing) the concentration or levels of the protein relative to the concentration or level of the protein in the absence of the activator. The terms may reference activation, or activating, sensitizing, or up-regulating signal transduction or enzymatic activity or the amount of a protein decreased in a disease. Thus, activation may include, at least in part, partially or totally increasing stimulation, increasing or enabling activation, or activating, sensitizing, or up-regulating signal transduction or enzymatic activity or the amount of a protein associated with a disease (e.g., a protein which is decreased in a disease relative to a non-diseased control). Activation may include, at least in part, partially or totally increasing stimulation, increasing or enabling activation, or activating, sensitizing, or up-regulating signal transduction or enzymatic activity or the amount of a protein

The terms “agonist,” “activator,” “upregulator,” etc. refer to a substance capable of detectably increasing the expression or activity of a given gene or protein. The agonist can increase expression or activity 10%, 20%, 30%, 40%, 50%, 60%, 70%, 80%, 90% or more in comparison to a control in the absence of the agonist. In certain instances, expression or activity is 1.5-fold, 2-fold, 3-fold, 4-fold, 5-fold, 10-fold or higher than the expression or activity in the absence of the agonist.

As defined herein, the term “inhibition”, “inhibit”, “inhibiting” and the like in reference to a protein-inhibitor interaction means negatively affecting (e.g., decreasing) the activity or function of the protein relative to the activity or function of the protein in the absence of the inhibitor. In embodiments inhibition means negatively affecting (e.g., decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

A “Notch inhibitor” refers to a compound (e.g., a compound described herein) that decreases the activity of Notch (e.g., Notch intracellular domain (NICD), Notch 1, Notch 2, Notch 3, or Notch 4; or intracellular domain thereof), level of activity of Notch (e.g., Notch intracellular domain (NICD), level of activity of Notch Transcription Complex (NTC), level of NTC, level of activity of Notch 1, level of activity of Notch 2, level of activity of Notch 3, or level of activity of Notch 4; or level of activity of intracellular domain thereof) when compared to a control, such as absence of the compound or a compound with known inactivity.

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

The term “Notch” refers to one or more (e.g., 1, 2, 3, or 4) of the four human transcription factors Notch 1, Notch 2, Notch 3, and/or Notch 4. The term includes any recombinant or naturally-occurring form of Notch (e.g., one or more of Notch 1, Notch 2, Notch 3, and/or Notch 4), including variants thereof that maintain Notch (e.g., one or more of Notch 1, Notch 2, Notch 3, and/or Notch 4) function or activity (e.g., within at least 30%, 40%, 50%, 60%, 70%, 80%, 90%, 95%, or 100% function or activity compared to wildtype Notch (e.g., one or more of Notch 1, Notch 2, Notch 3, and/or Notch 4)). In embodiments, Notch (e.g., one or more of Notch 1, Notch 2, Notch 3, and/or Notch 4) protein is a cleaved form of the full length protein. In embodiments, the Notch (e.g., one or more of Notch 1, Notch 2, Notch 3, and/or Notch 4) protein is the intracellular domain of the full length protein. In embodiments, Notch refers to Notch 1. In embodiments, Notch refers to Notch 2. In embodiments, Notch refers to Notch 3. In embodiments, Notch refers to Notch 4.

The terms “Notch homolog 1”, “Notch1”, and “Notch 1”,refer to the human transcription factor Notch1. The term includes any recombinant or naturally-occurring form of Notch1, including variants thereof that maintain Notch1 function or activity (e.g., within at least 30%, 40%, 50%, 60%, 70%, 80%, 90%, 95%, or 100% function or activity compared to wildtype Notch1). In embodiments, Notch1 is encoded by the NOTCH1 gene. In embodiments, Notch1 has the amino acid sequence set forth in or corresponding to Entrez 4851, UniProt P46531, or RefSeq (protein) NP_060087. In embodiments, Notch1 has the amino acid sequence set forth in or corresponding to RefSeq (protein) NP_060087.3. In embodiments, the Notch1 protein is a cleaved form of the full length protein. In embodiments, the Notch1 protein is the intracellular domain of the full length protein.

The terms “Notch homolog 2”, “Notch2”, “Neurogenic locus notch homolog protein 2”, and “Notch 2”,refer to the human transcription factor Notch2. The term includes any recombinant or naturally-occurring form of Notch2, including variants thereof that maintain Notch2 function or activity (e.g., within at least 30%, 40%, 50%, 60%, 70%, 80%, 90%, 95%, or 100% function or activity compared to wildtype Notch2). In embodiments, Notch2 is encoded by the NOTCH2 gene. In embodiments, Notch2 has the amino acid sequence set forth in or corresponding to Entrez 4853, UniProt Q04721, or RefSeq (protein) NP_077719. In embodiments, Notch2 has the amino acid sequence set forth in or corresponding to RefSeq (protein) NP_077719.2. In embodiments, the Notch2 protein is a cleaved form of the full length protein. In embodiments, the Notch2 protein is the intracellular domain of the full length protein.

The term “Notch homolog 3”, “Notch3”, “Neurogenic locus notch homolog protein 3”, and “Notch 3”,refer to the human transcription factor Notch3. The term includes any recombinant or naturally-occurring form of Notch3, including variants thereof that maintain Notch3 function or activity (e.g., within at least 30%, 40%, 50%, 60%, 70%, 80%, 90%, 95%, or 100% function or activity compared to wildtype Notch3). In embodiments, Notch3 is encoded by the NOTCH3 gene. In embodiments, Notch3 has the amino acid sequence set forth in or corresponding to Entrez 4854, UniProt Q9UM47, or RefSeq (protein) NP_000426. In embodiments, Notch3 has the amino acid sequence set forth in or corresponding to RefSeq (protein) NP_000426.2. In embodiments, the Notch3 protein is a cleaved form of the full length protein. In embodiments, the Notch3 protein is the intracellular domain of the full length protein.

The term “Notch homolog 4”, “Notch4”, “Neurogenic locus notch homolog protein 4”, and “Notch 4”,refer to the human transcription factor Notch4. The term includes any recombinant or naturally-occurring form of Notch4, including variants thereof that maintain Notch4 function or activity (e.g., within at least 30%, 40%, 50%, 60%, 70%, 80%, 90%, 95%, or 100% function or activity compared to wildtype Notch4). In embodiments, Notch4 is encoded by the NOTCH4 gene. In embodiments, Notch4 has the amino acid sequence set forth in or corresponding to Entrez 4855, UniProt Q99466, or RefSeq (protein) NP_004548. In embodiments, Notch4 has the amino acid sequence set forth in or corresponding to RefSeq (protein) NP_004548.3. In embodiments, the Notch4 protein is a cleaved form of the full length protein. In embodiments, the Notch4 protein is the intracellular domain of the full length protein.

The term “Recombination signal binding protein for immunoglobulin kappa J region”, “RBPJ”, “CSL”, and “CBF1” refer to the human protein RBPJ, which is the human homolog of of the Drosophila gene Suppressor of Hairless. The term includes any recombinant or naturally-occurring form of CSL, including variants thereof that maintain CSL function or activity (e.g., within at least 30%, 40%, 50%, 60%, 70%, 80%, 90%, 95%, or 100% function or activity compared to wildtype CSL). In embodiments, CSL is encoded by the RBPJ gene. In embodiments, CSL has the amino acid sequence set forth in or corresponding to Entrez 3516, UniProt Q06330, or RefSeq (protein) NP_005340. In embodiments, CSL has the amino acid sequence set forth in or corresponding to RefSeq (protein) NP_005340.2.

The term “Mastermind”, “Mastermind-like protein 1”, and “MAML1” refer to the human protein Mastermind-like protein 1. The term includes any recombinant or naturally-occurring form of Mastermind, including variants thereof that maintain Mastermind function or activity (e.g., within at least 30%, 40%, 50%, 60%, 70%, 80%, 90%, 95%, or 100% function or activity compared to wildtype Mastermind). In embodiments, Mastermind is encoded by the MAML1 gene. In embodiments, Mastermind has the amino acid sequence set forth in or corresponding to Entrez 9794, UniProt Q92585, or RefSeq (protein) NP_055572. In embodiments, Mastermind has the amino acid sequence set forth in or corresponding to RefSeq (protein) NP_055572.1.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

The term “modulator” refers to a composition that increases or decreases the level of a target molecule or the function of a target molecule or the physical state of the target of the molecule relative to the absence of the modulator. In some embodiments, a Notch (e.g., one or more of Notch 1, Notch 2, Notch 3, and/or Notch 4) associated disease modulator is a compound that reduces the severity of one or more symptoms of a disease associated with Notch (e.g., one or more of Notch 1, Notch 2, Notch 3, and/or Notch 4) (e.g. cancer). A Notch (e.g., one or more of Notch 1, Notch 2, Notch 3, and/or Notch 4) modulator is a compound that increases or decreases the activity or function or level of activity or level of function of Notch (e.g., one or more of Notch 1, Notch 2, Notch 3, and/or Notch 4). In some embodiments, a Notch (e.g., one or more of Notch 1, Notch 2, Notch 3, and/or Notch 4) associated disease modulator is a compound that reduces the severity of one or more symptoms of a disease associated with Notch (e.g., one or more of Notch 1, Notch 2, Notch 3, and/or Notch 4) (e.g. cancer). A Notch (e.g., one or more of Notch 1, Notch 2, Notch 3, and/or Notch 4) modulator is a compound that increases or decreases the activity or function or level of activity or level of function of Notch (e.g., one or more of Notch 1, Notch 2, Notch 3, and/or Notch 4).

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The term “associated” or “associated with” in the context of a substance or substance activity or function associated with a disease (e.g. a protein associated disease, a cancer associated with Notch (e.g., one or more of Notch 1, Notch 2, Notch 3, and/or Notch 4) activity, Notch (e.g., one or more of Notch 1, Notch 2, Notch 3, and/or Notch 4) associated cancer, Notch (e.g., one or more of Notch 1, Notch 2, Notch 3, and/or Notch 4) associated disease (e.g., cancer)) means that the disease (e.g., cancer) is caused by (in whole or in part), or a symptom of the disease is caused by (in whole or in part) the substance or substance activity or function. For example, a cancer associated with Notch (e.g., one or more of Notch 1, Notch 2, Notch 3, and/or Notch 4) activity or function may be a cancer that results (entirely or partially) from aberrant Notch (e.g., one or more of Notch 1, Notch 2, Notch 3, and/or Notch 4) function (e.g. enzyme activity, protein-protein interaction, signaling pathway) or a cancer wherein a particular symptom of the disease is caused (entirely or partially) by aberrant Notch (e.g., one or more of Notch 1, Notch 2, Notch 3, and/or Notch 4) activity or function. As used herein, what is described as being associated with a disease, if a causative agent, could be a target for treatment of the disease. For example, a cancer associated with Notch (e.g., one or more of Notch 1, Notch 2, Notch 3, and/or Notch 4) activity or function or a Notch (e.g., one or more of Notch 1, Notch 2, Notch 3, and/or Notch 4) associated disease (e.g., cancer), may be treated with a Notch (e.g., one or more of Notch 1, Notch 2, Notch 3, and/or Notch 4) modulator or Notch (e.g., one or more of Notch 1, Notch 2, Notch 3, and/or Notch 4) inhibitor, in the instance where increased Notch (e.g., one or more of Notch 1, Notch 2, Notch 3, and/or Notch 4) activity or function (e.g., signaling pathway activity) causes the disease (e.g., cancer). A cancer associated with Notch (e.g., one or more of Notch 1, Notch 2, Notch 3, and/or Notch 4) activity or function or a Notch (e.g., one or more of Notch 1, Notch 2, Notch 3, and/or Notch 4) associated disease (e.g., cancer), may be treated with a Notch (e.g., one or more of Notch 1, Notch 2, Notch 3, and/or Notch 4) modulator or Notch (e.g., one or more of Notch 1, Notch 2, Notch 3, and/or Notch 4) activator, in the instance where decreased Notch (e.g., one or more of Notch 1, Notch 2, Notch 3, and/or Notch 4) activity or function (e.g. signaling pathway activity) causes the disease (e.g., cancer).

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “signaling pathway” as used herein refers to a series of interactions between cellular and optionally extra-cellular components (e.g., proteins, nucleic acids, small molecules, ions, lipids) that conveys a change in one component to one or more other components, which in turn may convey a change to additional components, which is optionally propagated to other signaling pathway components. For example, binding of a Notch (e.g., one or more of Notch 1, Notch 2, Notch 3, and/or Notch 4) with a compound as described herein may reduce the interactions between the Notch (e.g., one or more of Notch 1, Notch 2, Notch 3, and/or Notch 4) and downstream effectors or signaling pathway components, resulting in changes in cell growth, proliferation, or survival.

In this disclosure, “comprises,” “comprising,” “containing” and “having” and the like can have the meaning ascribed to them in U.S. Patent law and can mean “includes,” “including,” and the like. “Consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The terms “disease” or “condition” refer to a state of being or health status of a patient or subject capable of being treated with the compounds or methods provided herein. The disease may be a cancer. In some further instances, “cancer” refers to human cancers and carcinomas, sarcomas, adenocarcinomas, lymphomas, leukemias, etc., including solid and lymphoid cancers, kidney, breast, lung, bladder, colon, ovarian, prostate, pancreas, stomach, brain, head and neck, skin, uterine, testicular, glioma, esophagus, and liver cancer, including hepatocarcinoma, lymphoma, including B-acute lymphoblastic lymphoma, non-Hodgkin’s lymphomas (e.g., Burkitt’s, Small Cell, and Large Cell lymphomas), Hodgkin’s lymphoma, leukemia (including AML, ALL, and CML), or multiple myeloma.

As used herein, the term “cancer” refers to all types of cancer, neoplasm or malignant tumors found in mammals (e.g., humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin’s Disease, and Non-Hodgkin’s Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, acute lymphoblastic leukemia (ALL), chronic lymphocytic leukemia (CLL), acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, acute myeloid leukemia (AML), chronic myeloid leukemia (CML), leukemia cutis, embryonal leukemia, eosinophilic leukemia, Gross’ leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myelodysplastic syndrome (MDS), myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling’s leukemia, stem cell leukemia, subleukemic leukemia, or undifferentiated cell leukemia.

As used herein, the term “lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin’s disease. Hodgkin’s disease represents approximately 15% of all diagnosed lymphomas. This is a cancer associated with Reed-Sternberg malignant B lymphocytes. Non-Hodgkin’s lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma (MCL), follicular lymphoma, marginal zone B-cell lymphoma (MZL), mucosa-associated lymphatic tissue lymphoma (MALT), extranodal lymphoma, nodal (monocytoid B-cell) lymphoma, splenic lymphoma, diffuse large cell B-lymphoma (DLBCL), activated B-cell subtype diffuse large B-cell lymphoma (ABC-DBLCL), germinal center B-cell like diffuse large B-cell lymphoma, Burkitt’s lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cutaneous T-cell lymphoma, peripheral T-cell lymphoma, anaplastic large cell lymphoma, mycosis fungocides, and precursor T-lymphoblastic lymphoma.

The term “sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy’s sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms’ tumor sarcoma, endometrial sarcoma, stromal sarcoma, Ewing’s sarcoma, fascial sarcoma, fibroblastic sarcoma, giant cell sarcoma, granulocytic sarcoma, Hodgkin’s sarcoma, idiopathic multiple pigmented hemorrhagic sarcoma, immunoblastic sarcoma of B cells, lymphoma, immunoblastic sarcoma of T-cells, Jensen’s sarcoma, Kaposi’s sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” is taken to mean a tumor arising from the melanocytic system of the skin and other organs. Melanomas that may be treated with a compound or method provided herein include, for example, acral-lentiginous melanoma, amelanotic melanoma, benign juvenile melanoma, Cloudman’s melanoma, S91 melanoma, Harding-Passey melanoma, juvenile melanoma, lentigo maligna melanoma, malignant melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher’s carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

As used herein, the terms “metastasis,” “metastatic,” and “metastatic cancer” can be used interchangeably and refer to the spread of a proliferative disease or disorder, e.g., cancer, from one organ or another non-adjacent organ or body part. “Metastatic cancer” is also called “Stage IV cancer.” Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The terms “cutaneous metastasis” or “skin metastasis” refer to secondary malignant cell growths in the skin, wherein the malignant cells originate from a primary cancer site (e.g., breast). In cutaneous metastasis, cancerous cells from a primary cancer site may migrate to the skin where they divide and cause lesions. Cutaneous metastasis may result from the migration of cancer cells from breast cancer tumors to the skin.

The term “visceral metastasis” refer to secondary malignant cell growths in the internal organs (e.g., heart, lungs, liver, pancreas, intestines) or body cavities (e.g., pleura, peritoneum), wherein the malignant cells originate from a primary cancer site (e.g., head and neck, liver, breast). In visceral metastasis, cancerous cells from a primary cancer site may migrate to the internal organs where they divide and cause lesions. Visceral metastasis may result from the migration of cancer cells from liver cancer tumors or head and neck tumors to internal organs.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient’s physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject’s condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease’s transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease’s spread; relieve the disease’s symptoms (e.g., ocular pain, seeing halos around lights, red eye, very high intraocular pressure), fully or partially remove the disease’s underlying cause, shorten a disease’s duration, or do a combination of these things.

“Treating” and “treatment” as used herein include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient. In embodiments, the treating or treatment is not prophylactic treatment (e.g., the patient has a disease, the patient suffers from a disease).

The term “prevent” refers to a decrease in the occurrence of Notch (e.g., one or more of Notch 1, Notch 2, Notch 3, and/or Notch 4) associated disease symptoms or Notch (e.g., one or more of Notch 1, Notch 2, Notch 3, and/or Notch 4) associated disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual’s disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

“Co-administer” it is meant that a composition described herein is administered at the same time, just prior to, or just after the administration of one or more additional therapies. The compounds provided herein can be administered alone or can be coadministered to the patient. Coadministration is meant to include simultaneous or sequential administration of the compounds individually or in combination (more than one compound). Thus, the preparations can also be combined, when desired, with other active substances (e.g., to reduce metabolic degradation). The compositions of the present disclosure can be delivered transdermally, by a topical route, or formulated as applicator sticks, solutions, suspensions, emulsions, gels, creams, ointments, pastes, jellies, paints, powders, and aerosols.

“Anti-cancer agent” is used in accordance with its plain ordinary meaning and refers to a composition (e.g., compound, drug, antagonist, inhibitor, modulator) having antineoplastic properties or the ability to inhibit the growth or proliferation of cells. In some embodiments, an anti-cancer agent is a chemotherapeutic. In some embodiments, an anti-cancer agent is an agent identified herein having utility in methods of treating cancer. In some embodiments, an anti-cancer agent is an agent approved by the FDA or similar regulatory agency of a country other than the USA, for treating cancer. In embodiments, an anti-cancer agent is an agent with antineoplastic properties that has not (e.g., yet) been approved by the FDA or similar regulatory agency of a country other than the USA, for treating cancer. Examples of anti-cancer agents include, but are not limited to, MEK (e.g., MEK1, MEK2, or MEK1 and MEK2) inhibitors (e.g., XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5- azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g., cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g., U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec.RTM.), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL.sub.2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g., Taxol.TM (i.e., paclitaxel), Taxotere.TM, compounds comprising the taxane skeleton, Erbulozole (i.e., R-55104), Dolastatin 10 (i.e., DLS-10 and NSC-376128), Mivobulin isethionate (i.e., as CI-980), Vincristine, NSC-639829, Discodermolide (i.e., as NVP-XX-A-296), ABT-751 (Abbott, i.e., E-7010), Altorhyrtins (e.g., Altorhyrtin A and Altorhyrtin C), Spongistatins (e.g., Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e., LU-103793 and NSC-D-669356), Epothilones (e.g., Epothilone A, Epothilone B, Epothilone C (i.e., desoxyepothilone A or dEpoA), Epothilone D (i.e., KOS-862, dEpoB, and desoxyepothilone B), Epothilone E, Epothilone F, Epothilone B N-oxide, Epothilone A N-oxide, 16-aza-epothilone B, 21-aminoepothilone B (i.e., BMS-310705), 21-hydroxyepothilone D (i.e., Desoxyepothilone F and dEpoF), 26-fluoroepothilone, Auristatin PE (i.e., NSC-654663), Soblidotin (i.e., TZT-1027), LS-4559-P (Pharmacia, i.e., LS-4577), LS-4578 (Pharmacia, i.e., LS-477-P), LS-4477 (Pharmacia), LS-4559 (Pharmacia), RPR-112378 (Aventis), Vincristine sulfate, DZ-3358 (Daiichi), FR-182877 (Fujisawa, i.e., WS-9885B), GS-164 (Takeda), GS-198 (Takeda), KAR-2 (Hungarian Academy of Sciences), BSF-223651 (BASF, i.e., ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e., LY-355703), AC-7739 (Ajinomoto, i.e., AVE-8063A and CS-39.HCl), AC-7700 (Ajinomoto, i.e., AVE-8062, AVE-8062A, CS-39-L-Ser.HCl, and RPR-258062A), Vitilevuamide, Tubulysin A, Canadensol, Centaureidin (i.e., NSC-106969), T-138067 (Tularik, i.e., T-67, TL-138067 and TI-138067), COBRA-1 (Parker Hughes Institute, i.e., DDE-261 and WHI-261), H10 (Kansas State University), H16 (Kansas State University), Oncocidin A1 (i.e., BTO-956 and DIME), DDE-313 (Parker Hughes Institute), Fijianolide B, Laulimalide, SPA-2 (Parker Hughes Institute), SPA-1 (Parker Hughes Institute, i.e., SPIKET-P), 3-IAABU (Cytoskeleton/Mt. Sinai School of Medicine, i.e., MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e., MF-191), TMPN (Arizona State University), Vanadocene acetylacetonate, T-138026 (Tularik), Monsatrol, Inanocine (i.e., NSC-698666), 3-IAABE (Cytoskeleton/Mt. Sinai School of Medicine), A-204197 (Abbott), T-607 (Tuiarik, i.e., T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (-)-Phenylahistin (i.e., NSCL-96F037), D-68838 (Asta Medica), D-68836 (Asta Medica), Myoseverin B, D-43411 (Zentaris, i.e., D-81862), A-289099 (Abbott), A-318315 (Abbott), HTI-286 (i.e., SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to ¹¹¹In, ⁹⁰Y, or ¹³¹I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g., gefitinib (Iressa™), erlotinib (Tarceva™), cetuximab (Erbitux™), lapatinib (Tykerb™), panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like. A moiety of an anti-cancer agent is a monovalent anti-cancer agent (e.g., a monovalent form of an agent listed above).

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples).

“CSL-Notch-Mastermind complex” is used in accordance with its well understood meaning in biology and refers to the protein complex including the proteins CSL, Notch (e.g., one or more of Notch 1, Notch 2, Notch 3, and/or Notch 4), and Mastermind, which may each interact with one or both of the other proteins either directly or indirectly through another component of the complex. In embodiments, the CSL-Notch (e.g., one or more of Notch 1, Notch 2, Notch 3, and/or Notch 4)-Mastermind complex modulates transcription. The Notch (e.g., one or more of Notch 1, Notch 2, Notch 3, and/or Notch 4) protein included in the CSL-Notch-Mastermind complex may be an intracellular portion of the full length Notch (e.g., one or more of Notch 1, Notch 2, Notch 3, and/or Notch 4) receptor. In embodiments, the Notch in the CSL-Notch-Mastermind complex is Notch 1. In embodiments, the Notch in the CSL-Notch-Mastermind complex is Notch 2. In embodiments, the Notch in the CSL-Notch-Mastermind complex is Notch 3. In embodiments, the Notch in the CSL-Notch-Mastermind complex is Notch 4.

### II. Compounds

In an aspect is provided a compound having the formula:

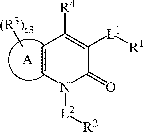

or a salt (e.g., pharmaceutically acceptable salt) thereof.

L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene (e.g., C₁—C₈, C₁—C₆, or C₁—C₄) or substituted or unsubstituted heteroalkylene (e.g., 2 to 8 membered, 2 to 6 membered, or 2 to 4 membered).

R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R¹²)—, —N(R^{L2})SO₂—, substituted or unsubstituted alkylene (e.g., C₁—C₈, C₁—C₆, or C₁—C₄) or substituted or unsubstituted heteroalkylene (e.g., 2 to 8 membered, 2 to 6 membered, or 2 to 4 membered).

R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

Ring A is C₅—C₆ cycloalkyl, 5 to 6 membered heterocycloalkyl, phenyl, or 5 to 6 membered heteroaryl.

R³ is independently halogen, oxo, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); two adjacent R³ substituents may optionally be joined to form a substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

z3 is independently an integer from 0 to 8.

R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D}.

R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered) ; R^{2A} and R^{2B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered) ; R^{3A} and R^{3B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered) ; R^{4A} and R^{4B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

X¹, X², X³, and X⁴ are independently —F, —Cl, —Br, or —I.

n1, n2, and n3 are independently an integer from 0 to 4.

m1, m2, m3, v1, v2, and v3 are independently 1 or 2.

In embodiments, Ring A is C₅—C₆ cycloalkyl. In embodiments, Ring A is C₅ cycloalkyl. In embodiments, Ring A is C₆ cycloalkyl. In embodiments, Ring A is C₅ cycloalkenyl. In embodiments, Ring A is C₆ cycloalkenyl. In embodiments, Ring A is 5 to 6 membered heterocycloalkyl. In embodiments, Ring A is 5 membered heterocycloalkyl. In embodiments, Ring A is 6 membered heterocycloalkyl. In embodiments, Ring A is 5 membered heterocycloalkenyl. In embodiments, Ring A is 6 membered heterocycloalkenyl.

In embodiments, Ring A is phenyl. In embodiments, Ring A is a 5 to 6 membered heteroaryl. In embodiments, Ring A is a 5 membered heteroaryl. In embodiments, Ring A is a 6 membered heteroaryl. In embodiments, Ring A is pyridyl. In embodiments, Ring A is pyrazinyl. In embodiments, Ring A is pyridazinyl. In embodiments, Ring A is pyrimidinyl. In embodiments, Ring A is triazinyl.

In embodiments, the compound has the formula:

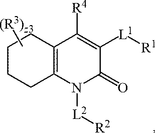

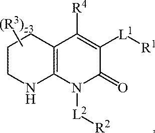

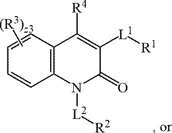

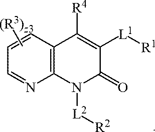

R¹, L¹, R², L², R³, R⁴, and z3 are as described herein, including in embodiments. In embodiments, the compound has the formula:

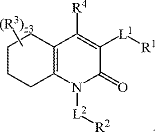

R¹, L¹, R², L², R³, R⁴, and z3 are as described herein, including in embodiments. In embodiments, the compound has the formula:

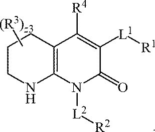

R¹, L¹, R², L², R³, R⁴, and z3 are as described herein, including in embodiments. In embodiments, the compound has the formula:

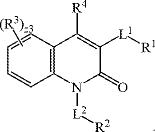

R¹, L¹, R², L², R³, R⁴, and z3 are as described herein, including in embodiments. In embodiments, the compound has the formula:

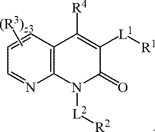

R¹, L¹, R², L², R³, R⁴, and z3 are as described herein, including in embodiments.

In embodiments, the compound has the formula:

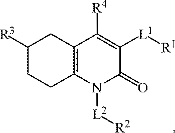

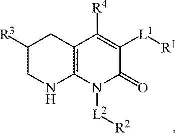

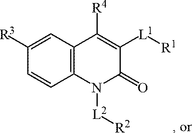

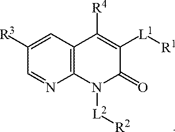

R¹, L¹, R², L², R³, and R⁴ are as described herein, including in embodiments. In embodiments, the compound has the formula:

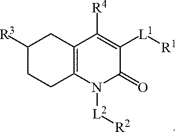

R¹, L¹, R², L², R³, and R⁴ are as described herein, including in embodiments. In embodiments, the compound has the formula:

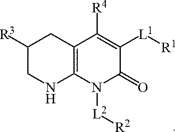

R¹, L¹, R², L², R³, and R⁴ are as described herein, including in embodiments. In embodiments, the compound has the formula:

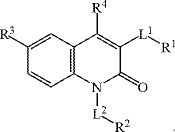

R¹, L¹, R², L², R³, and R⁴ are as described herein, including in embodiments. In embodiments, the compound has the formula:

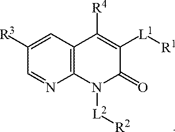

R¹, L¹, R², L², R³, and R⁴ are as described herein, including in embodiments.

In embodiments, the compound has the formula:

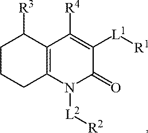

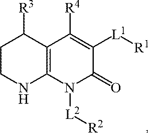

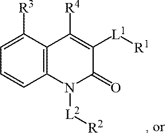

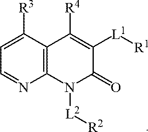

R¹, L¹, R², L², R³, and R⁴ are as described herein, including in embodiments. In embodiments, the compound has the formula:

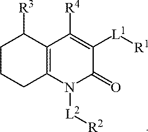

R¹, L¹, R², L², R³, and R⁴ are as described herein, including in embodiments. In embodiments, the compound has the formula:

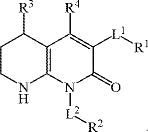

R¹, L¹, R², L², R³, and R⁴ are as described herein, including in embodiments. In embodiments, the compound has the formula:

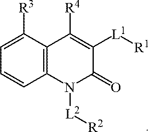

R¹, L¹, R², L², R³, and R⁴ are as described herein, including in embodiments. In embodiments, the compound has the formula:

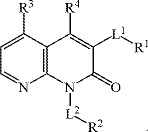

R¹, L¹, R², L², R³, and R⁴ are as described herein, including in embodiments.

In embodiments, the compound has the formula:

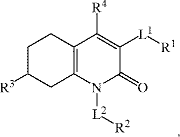

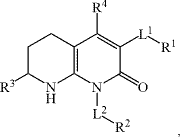

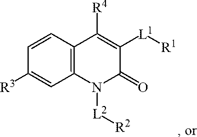

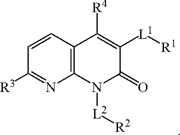

R¹, L¹, R², L², R³, and R⁴ are as described herein, including in embodiments. In embodiments, the compound has the formula:

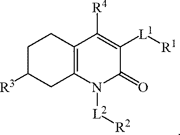

R¹, L¹, R², L², R³, and R⁴ are as described herein, including in embodiments. In embodiments, the compound has the formula:

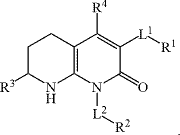

R¹, L¹, R², L², R³, and R⁴ are as described herein, including in embodiments. In embodiments, the compound has the formula:

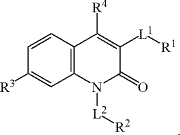

R¹, L¹, R², L², R³, and R⁴ are as described herein, including in embodiments. In embodiments, the compound has the formula:

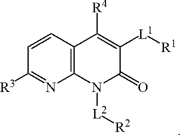

R¹, L¹, R², L², R³, and R⁴ are as described herein, including in embodiments.

In embodiments, the compound has the formula:

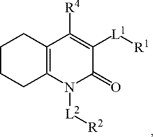

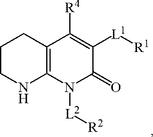

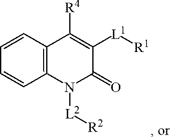

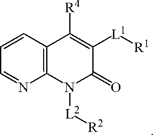

R¹, L¹, R², L², and R⁴ are as described herein, including in embodiments. In embodiments, the compound has the formula:

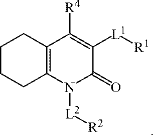

R¹, L¹, R², L², and R⁴ are as described herein, including in embodiments. In embodiments, the compound has the formula:

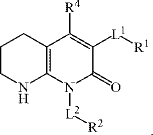

R¹, L¹, R², L², and R⁴ are as described herein, including in embodiments. In embodiments, the compound has the formula:

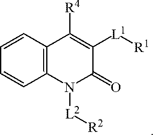

R¹, L¹, R², L², and R⁴ are as described herein, including in embodiments. In embodiments, the compound has the formula:

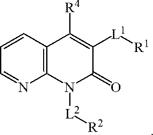

R¹, L¹, R², L², and R⁴ are as described herein, including in embodiments.

In embodiments, the compound has the formula:

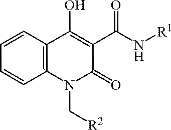

and R¹ and R² are as described herein, including in embodiments.

In embodiments, the compound has the formula:

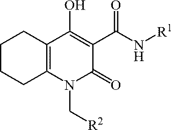

and R¹ and R² are as described herein, including in embodiments.

In embodiments, L¹ is a bond. In embodiments, L¹ is —N(R^{L1})—. In embodiments, L¹ is —O—. In embodiments, L¹ is —S—. In embodiments, L¹ is —SO₂—. In embodiments, L¹ is —C(O)—. In embodiments, L¹ is —C(O)N(R^{L1})—. In embodiments, L¹ is —N(R^{L1})C(O)—. In embodiments, L¹ is —N(R^{L1})C(O)NH—. In embodiments, L¹ is —NHC(O)N(R^{L1})—. In embodiments, L¹ is —C(O)O—. In embodiments, L¹ is —OC(O)—. In embodiments, L¹ is —SO₂N(R^{L1})—. In embodiments, L¹ is —N(R^{L1})SO₂—. In embodiments, L¹ is substituted or unsubstituted alkylene (e.g., C₁—C₈, C₁—C₆, or C₁—C₄). In embodiments, L¹ is substituted or unsubstituted heteroalkylene (e.g., 2 to 8 membered, 2 to 6 membered, or 2 to 4 membered). In embodiments, L¹ is a bond, —NH—, —O—, —S—, —SO₂—, —C(O)—, —C(O)NH—, —NHC(O)—, —NHC(O)NH—, —C(O)O—, —OC(O)—, —SO₂NH—, —NHSO₂—, substituted or unsubstituted C₁—C₆ alkylene, or, substituted or unsubstituted 2 to 6 membered heteroalkylene.

In embodiments, L¹ is substituted or unsubstituted heteroalkylene. In embodiments, L¹ is —C(O)N(R^{L1})—(C₁—C₆ alkyl)- or —SO₂N(R^{L1})—(C₁—C₆ alkyl)-. In embodiments, L¹ is —C(O)N(R^{L1})CH₂— or —SO₂N(R^{L1})CH₂—. In embodiments, L¹ is —C(O)N(R^{L1})CH₂—. In embodiments, L¹ is a substituted or unsubstituted alkylene. In embodiments, L¹ is an unsubstituted C₁—C₆ alkylene. In embodiments, L¹ is an unsubstituted methylene. In embodiments, L¹ is a substituted alkylene. In embodiments, L¹ is a substituted C₁—C₆ alkylene. In embodiments, L¹ is —C(O)—. In embodiments, L¹ is -C(O)N(R^{L1})(C₁—C₆ alkyl)-. In embodiments, L¹ is -SO₂N(R^{L1})(C₁—C₆ alkyl)--. In embodiments, L¹ is —C(O)N(R^{L1})CH₂—. In embodiments, L¹ is —SO₂N(R^{L1})CH₂—. In embodiments, L¹ is -C(O)NH(C₁—C₆ alkyl)-. In embodiments, L¹ is —SO₂NH—(C₁—C₆ alkyl)-. In embodiments, L¹ is —C(O)NHCH₂—. In embodiments, L¹ is —SO₂NHCH₂—. In embodiments, L¹ is -(C₁—C₆ alkyl)—C(O)N(R^{L1})— or -(C₁—C₆ alkyl)—SO₂N(R^{L1})—. In embodiments, L¹ is —CH₂C(O)N(R^{L1})— or —CH₂SO₂N(R^{L1})—. In embodiments, L¹ is -(C₁—C₆ alkyl)—C(O)N(R^{L1})—. In embodiments, L¹ is -(C₁—C₆ alkyl)—SO₂N(R^{L1})—. In embodiments, L¹ is —CH₂C(O)N(R^{L1})—. In embodiments, L¹ is —CH₂SO₂N(R^{L1})—. In embodiments, L¹ is -(C₁—C₆ alkyl)N(R^{L1})-. In embodiments, L¹ is —CH₂N(R^{L1})—. In embodiments, L¹ is -(C₁—C₆ alkyl)—C(O)NH—. In embodiments, L¹ is -(C₁-C₆ alkyl)—SO₂NH—. In embodiments, L¹ is —CH₂C(O)NH—. In embodiments, L¹ is —CH₂SO₂NH—. In embodiments, L¹ is -(C₁—C₆ alkyl)NH-. In embodiments, L¹ is —CH₂NH—. In embodiments, L¹ is —C(O)NH—, —NHC(O)—, —NHC(O)NH—, —SO₂NH—, —NHSO₂—, or substituted or unsubstituted heteroalkylene. In embodiments, L¹ is —C(O)NH—, —NHC(O)—, —NHC(O)NH—, —SO₂NH—, —NHSO₂—, or substituted or unsubstituted 2 to 3 membered heteroalkylene. In embodiments, L¹ is substituted or unsubstituted heteroalkylene. In embodiments, L¹ is substituted heteroalkylene. In embodiments, L¹ is unsubstituted heteroalkylene. In embodiments, L¹ is substituted or unsubstituted 2 to 3 membered heteroalkylene. In embodiments, L¹ is substituted 2 to 3 membered heteroalkylene. In embodiments, L¹ is unsubstituted 2 to 3 membered heteroalkylene.

In embodiments, L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, —N(R^{L1})CH₂—, —OCH₂—, —SCH₂—, —SO₂CH₂—, —C(O)CH₂—, —C(O)N(R^{L1})CH₂—, —N(R^{L1})C(O)CH₂—, —N(R^{L1})C(O)NHCH₂—, —NHC(O)N(R^{L1})CH₂—, —C(O)OCH₂—, —OC(O)CH₂—, —SO₂N(R^{L1})CH₂—, —N(R^{L1})SO₂CH₂—, —CH₂N(R^{L1})—, —CH₂O—, —CH₂S—, —CH₂SO₂—, —CH₂C(O)—, —CH₂C(O)N(R^{L1})—, —CH₂N(R^{L1})C(O)—, —CH₂N(R^{L1})C(O)NH—, —CH₂NHC(O)N(R^{L1})—, —CH₂C(O)O—, —CH₂OC(O)—, —CH₂SO₂N(R^{L1})—, or —CH₂N(R^{L1})SO₂—. In embodiments, L¹ is a bond. In embodiments, L¹ is —N(R^{L1})—. In embodiments, L¹ is —O—. In embodiments, L¹ is —S—. In embodiments, L¹ is —SO₂—. In embodiments, L¹ is —C(O)—. In embodiments, L¹ is —C(O)N(R^{L1})—. In embodiments, L¹ is —N(R^{L1})C(O)—. In embodiments, L¹ is —N(R^{L1})C(O)NH—. In embodiments, L¹ is —NHC(O)N(R^{L1})—. In embodiments, L¹ is —C(O)O—. In embodiments, L¹ is —OC(O)—. In embodiments, L¹ is —SO₂N(R^{L1})—. In embodiments, L¹ is —N(R^{L1})SO₂—. In embodiments, L¹ is —N(R^{L1})CH₂—. In embodiments, L¹ is —OCH₂—. In embodiments, L¹ is —SCH₂—. In embodiments, L¹ is —SO₂CH₂—. In embodiments, L¹ is —C(O)CH₂—. In embodiments, L¹ is —C(O)N(R^{L1})CH₂—. In embodiments, L¹ is —N(R^{L1})C(O)CH₂—. In embodiments, L¹ is —N(R^{L1})C(O)NHCH₂—. In embodiments, L¹ is —NHC(O)N(R^{L1})CH₂—. In embodiments, L¹ is —C(O)OCH₂—. In embodiments, L¹ is —OC(O)CH₂—. In embodiments, L¹ is —SO₂N(R^{L1})CH₂—. In embodiments, L¹ is —N(R^{L1})SO₂CH₂—. In embodiments, L¹ is —CH₂N(R^{L1})—. In embodiments, L¹ is —CH₂O—. In embodiments, L¹ is —CH₂S—. In embodiments, L¹ is —CH₂SO₂—. In embodiments, L¹ is —CH₂C(O)—. In embodiments, L¹ is —CH₂C(O)N(R^{L1})—. In embodiments, L¹ is —CH₂N(R^{L1})C(O)—. In embodiments, L¹ is —CH₂N(R^{L1})C(O)NH—. In embodiments, L¹ is —CH₂NHC(O)N(R^{L1})—. In embodiments, L¹ is —CH₂C(O)O—. In embodiments, L¹ is —CH₂OC(O)—. In embodiments, L¹ is —CH₂SO₂N(R^{L1})—. In embodiments, L¹ is —CH₂N(R^{L1})SO₂—. In embodiments, L¹ is —C(O)NH—, —NHC(O)—, —NHC(O)NH—, —SO₂NH—, —NHSO₂—, —NHCH₂—,—CH₂NH—, —C(O)NHCH₂—, or —NHC(O)CH₂—. In embodiments, L¹ is —C(O)NH—. In embodiments, L¹ is —NHC(O)—. In embodiments, L¹ is —NHC(O)NH—. In embodiments, L¹ is —SO₂NH—. In embodiments, L¹ is —NHSO₂—. In embodiments, L¹ is —NHCH₂—. In embodiments, L¹ is —CH₂NH—. In embodiments, L¹ is —C(O)NHCH₂—. In embodiments, L¹ is —NHC(O)CH₂—. In embodiments, L¹ is —C(O)N(R^{L1})— or —C(O)N(R^{L1})CH₂—. In embodiments, L¹ is —C(O)N(R^{L1})—. In embodiments, L¹ is —C(O)N(R^{L1})CH₂—. In embodiments, L¹ is —C(O)NH—. In embodiments, L¹ is —C(O)NHCH₂—. In embodiments, the right atom in the mainchain of the linker depicted for L¹ is directly bonded to R¹ (e.g., the —NH— of —C(O)NH— is directly bonded to R¹). In embodiments, the left atom in the mainchain of the linker depicted for L¹ is directly bonded to R¹ (e.g., the —C(O)— of —C(O)NH— is directly bonded to R¹).

In embodiments, R^{L1} is independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, unsubstituted alkyl, or unsubstituted cycloalkyl. In embodiments, R^{L1} is independently hydrogen, unsubstituted C₁—C₆ alkyl, or unsubstituted C₃—C₆ cycloalkyl. In embodiments, R^{L1} is independently hydrogen, unsubstituted methyl, unsubstituted ethyl, unsubstituted isopropyl, or unsubstituted cyclopropyl. In embodiments, R^{L1} is independently hydrogen. In embodiments, R^{L1} is independently hydrogen. In embodiments, R^{L1} is independently unsubstituted methyl. In embodiments, R^{L1} is independently unsubstituted ethyl. In embodiments, R^{L1} is independently unsubstituted isopropyl. In embodiments, R^{L1} is independently unsubstituted cyclopropyl.

In embodiments, R¹ is independently substituted or unsubstituted aryl or substituted or unsubstituted heteroaryl. In embodiments, R¹ is independently substituted phenyl or substituted 5 to 6 membered heteroaryl.

In embodiments, R¹ is independently hydrogen, oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R¹ is independently substituted or unsubstituted C₁—C₆ alkyl, substituted or unsubstituted 2 to 6 membered heteroalkyl, substituted or unsubstituted C₃—C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R¹ is independently —CCl₃. In embodiments, R¹ is independently —CBr₃. In embodiments, R¹ is independently —CF₃. In embodiments, R¹ is independently —CI₃. In embodiments, R¹ is independently —CHCl₂. In embodiments, R¹ is independently —CHBr₂. In embodiments, R¹ is independently —CHF₂. In embodiments, R¹ is independently —CHI₂. In embodiments, R¹ is independently —CH₂Cl. In embodiments, R¹ is independently —CH₂Br. In embodiments, R¹ is independently —CH₂F. In embodiments, R¹ is independently —CH₂I. In embodiments, R¹ is independently —CN. In embodiments, R¹ is independently —OH. In embodiments, R¹ is independently —NH₂. In embodiments, R¹ is independently —COOH. In embodiments, R¹ is independently —CONH₂. In embodiments, R¹ is independently —OCCl₃. In embodiments, R¹ is independently —OCF₃. In embodiments, R¹ is independently —OCBr₃. In embodiments, R¹ is independently —OCI₃. In embodiments, R¹ is independently —OCHCl₂. In embodiments, R¹ is independently —OCHBr₂. In embodiments, R¹ is independently —OCHI2. In embodiments, R¹ is independently —OCHF₂. In embodiments, R¹ is independently —OCH₂Cl. In embodiments, R¹ is independently —OCH₂Br. In embodiments, R¹ is independently —OCH₂I. In embodiments, R¹ is independently —OCH₂F. In embodiments, R¹ is independently halogen. In embodiments, R¹ is independently —NO₂. In embodiments, R¹ is independently —OCH₃. In embodiments, R¹ is independently —OCH₂CH₃. In embodiments, R¹ is independently —OCH(CH₃)₂. In embodiments, R¹ is independently —OC(CH₃)₃. In embodiments, R¹ is independently —CH₃. In embodiments, R¹ is independently —CH₂CH₃. In embodiments, R¹ is independently —CH(CH₃)₂. In embodiments, R¹ is independently —C(CH₃)₃. In embodiments, R¹ is independently unsubstituted cyclopropyl. In embodiments, R¹ is independently unsubstituted cyclobutyl. In embodiments, R¹ is independently unsubstituted cyclopentyl. In embodiments, R¹ is independently unsubstituted cyclohexyl.

In embodiments, R¹ is independently hydrogen. In embodiments, R¹ is independently oxo. In embodiments, R¹ is independently halogen. In embodiments, R¹ is independently —CX¹₃. In embodiments, R¹ is independently —CHX¹₂. In embodiments, R¹ is independently —CH₂X¹. In embodiments, R¹ is independently —OCX¹₃. In embodiments, R¹ is independently —OCH₂X¹. In embodiments, R¹ is independently —OCHX¹₂. In embodiments, R¹ is independently —CN. In embodiments, R¹ is independently —SF₅. In embodiments, R¹ is independently —N₃. In embodiments, R¹ is independently -SOₙ₁R^{1D}. In embodiments, R¹ is independently -SOᵥ₁NR^{1A}R^{1B}. In embodiments, R¹ is independently -NR^{1C}NR^{1A}R^{1B}. In embodiments, R¹ is independently —ONR^{1A}R^{1B}. In embodiments, R¹ is independently —NHC(O)NR^{1C}NR^{1A}R^{1B}. In embodiments, R¹ is independently —NHC(O)NR^{1A}R^{1B}. In embodiments, R¹ is independently —N(O)ₘ₁. In embodiments, R¹ is independently —NR^{1A}R^{1B}. In embodiments, R¹ is independently —C(O)R^{1C}. In embodiments, R¹ is independently —C(O)—OR^{1C}. In embodiments, R¹ is independently —C(O)NR^{1A}R^{1B}. In embodiments, R¹ is independently —OR^{1D}. In embodiments, R¹ is independently —NR^{1A}SO₂R^{1D}. In embodiments, R¹ is independently —NR^{1A}C(O)R^{1C}. In embodiments, R¹ is independently —NR^{1A}C(O)OR^{1C}. In embodiments, R¹ is independently -NR^{1A}OR^{1C}.

In embodiments, R¹ is independently substituted or unsubstituted alkyl (e.g., C₁—C₈, C₁—C₆, or C₁—C₄). In embodiments, R¹ is independently substituted or unsubstituted heteroalkyl (e.g., 2 to 8 membered, 2 to 6 membered, or 2 to 4 membered). In embodiments, R¹ is independently substituted or unsubstituted cycloalkyl (e.g., C₃—C₈, C₃—C₆, or C₅—C₆). In embodiments, R¹ is independently substituted or unsubstituted heterocycloalkyl (e.g., 3 to 8 membered, 3 to 6 membered, or 5 to 6 membered). In embodiments, R¹ is independently substituted or unsubstituted aryl (e.g., C₆—C₁₀, C₁₀, or phenyl). In embodiments, R¹ is independently substituted or unsubstituted heteroaryl (e.g., 5 to 10 membered, 5 to 9 membered, or 5 to 6 membered).

In embodiments, R¹ is independently R¹⁰-substituted or unsubstituted alkyl (e.g., C₁-C₈, C₁—C₆, or C₁—C₄). In embodiments, R¹ is independently R¹⁰-substituted or unsubstituted heteroalkyl (e.g., 2 to 8 membered, 2 to 6 membered, or 2 to 4 membered). In embodiments, R¹ is independently R¹⁰-substituted or unsubstituted cycloalkyl (e.g., C₃—C₈, C₃—C₆, or C₅—C₆). In embodiments, R¹ is independently R¹⁰-substituted or unsubstituted heterocycloalkyl (e.g., 3 to 8 membered, 3 to 6 membered, or 5 to 6 membered). In embodiments, R¹ is independently R¹⁰-substituted or unsubstituted aryl (e.g., C₆—C₁₀, C₁₀, or phenyl). In embodiments, R¹ is independently R¹⁰-substituted or unsubstituted heteroaryl (e.g., 5 to 10 membered, 5 to 9 membered, or 5 to 6 membered).

In embodiments, R¹ is independently R¹⁰-substituted or unsubstituted C₁—C₆ alkyl, R¹⁰-substituted or unsubstituted 2 to 6 membered heteroalkyl, R¹⁰-substituted or unsubstituted C₃—C₆ cycloalkyl, R¹⁰-substituted or unsubstituted 3 to 6 membered heterocycloalkyl, R¹⁰-substituted or unsubstituted phenyl, or R¹⁰-substituted or unsubstituted 5 to 6 membered heteroaryl. In embodiments, R¹ is independently R¹⁰-substituted or unsubstituted C₁—C₆ alkyl. In embodiments, R¹ is independently R¹⁰-substituted or unsubstituted 2 to 6 membered heteroalkyl. In embodiments, R¹ is independently R¹⁰-substituted or unsubstituted C₃—C₆ cycloalkyl. In embodiments, R¹ is independently R¹⁰-substituted or unsubstituted 3 to 6 membered heterocycloalkyl. In embodiments, R¹ is independently R¹⁰-substituted or unsubstituted phenyl. In embodiments, R¹ is independently or R¹⁰-substituted or unsubstituted 5 to 6 membered heteroaryl.

In embodiments, R¹ is independently R¹⁰-substituted or unsubstituted C₃—C₆ cycloalkyl, R¹⁰-substituted or unsubstituted 3 to 6 membered heterocycloalkyl, R¹⁰-substituted or unsubstituted phenyl, or R¹⁰-substituted or unsubstituted 5 to 6 membered heteroaryl.

In embodiments, R¹ is independently R¹⁰-substituted phenyl or R¹⁰-substituted 5 to 6 membered heteroaryl. In embodiments, R¹ is independently R¹⁰-substituted phenyl. In embodiments, R¹ is independently R¹⁰-substituted 5 to 6 membered heteroaryl. In embodiments, R¹ is independently

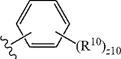

and R¹⁰ is as described herein and z10 is independently an integer from 0 to 5. z10 is independently an integer from 0 to 9. In embodiments, z10 is independently 0. In embodiments, z10 is independently 1. In embodiments, z10 is independently 2. In embodiments, z10 is independently 3. In embodiments, z10 is independently 4. In embodiments, z10 is independently 5. In embodiments, z10 is independently 6. In embodiments, z10 is independently 7. In embodiments, z10 is independently 8. In embodiments, z10 is independently 9. In embodiments, z10 is independently an integer from 0 to 5. In embodiments, R¹ is independently

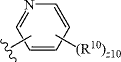

and R¹⁰ is as described herein and z10 is independently an integer from 0 to 4. In embodiments, R¹ is independently

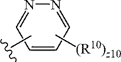

and R¹⁰ is as described herein and z10 is independently an integer from 0 to 3. In embodiments, R¹ is independently

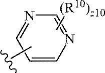

and R¹⁰ is as described herein and z10 is independently an integer from 0 to 3. In embodiments, R¹ is independently

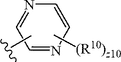

and R¹⁰ is as described herein and z10 is independently an integer from 0 to 3. In embodiments, R¹ is independently

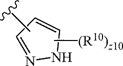

and R¹⁰ is as described herein and z10 is independently an integer from 0 to 3.

In embodiments, R¹ is

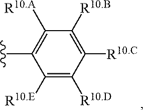

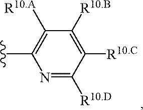

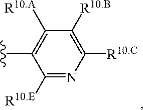

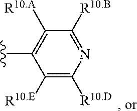

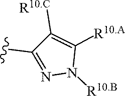

and R^{10.A}, R^{10.B}, R^{10.C}, R^{10.D}, and R^{10.E} are independently hydrogen or any value of R¹⁰ described herein, including in embodiments. In embodiments, R¹ is

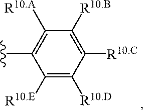

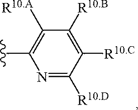

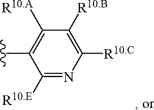

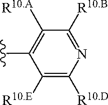

and R^{10.A}, R^{10.B}, R^{10.C}, R^{10.D}, and R^{10.E} are independently hydrogen or any value of R¹⁰ described herein, including in embodiments.

In embodiments, R¹ is independently

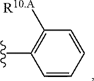

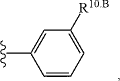

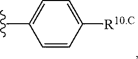

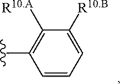

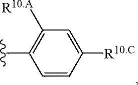

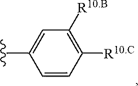

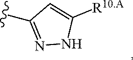

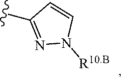

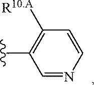

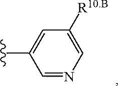

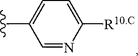

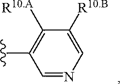

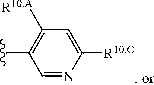

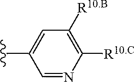

and R^{10.A}, R^{10.B}, and R^{10.C} are independently hydrogen or any value of R¹⁰ described herein, including in embodiments.

In embodiments, R¹ is independently

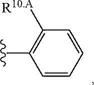

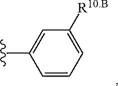

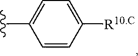

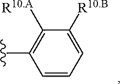

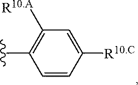

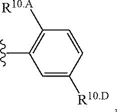

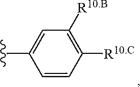

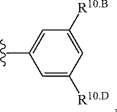

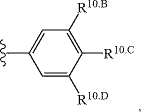

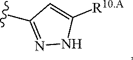

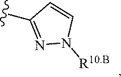

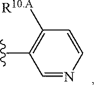

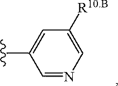

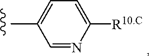

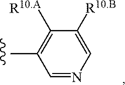

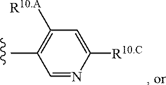

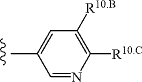

and R^{10.A}, R^{10.B}, R¹⁰^{.C}, and R^{10.D} are independently hydrogen or any value of R¹⁰ described herein, including in embodiments.

In embodiments, R¹ is independently

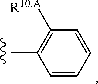

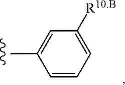

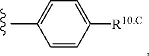

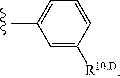

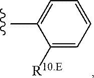

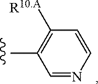

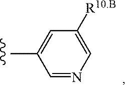

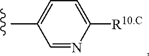

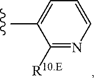

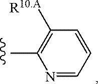

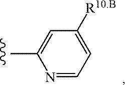

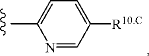

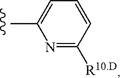

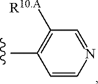

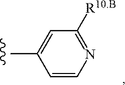

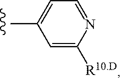

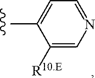

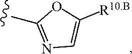

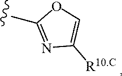

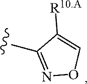

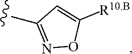

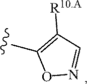

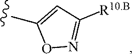

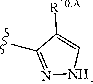

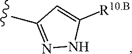

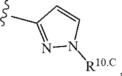

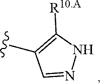

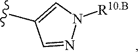

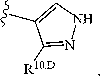

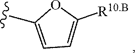

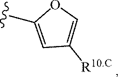

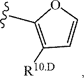

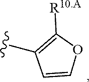

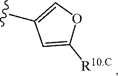

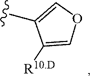

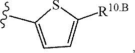

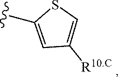

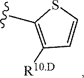

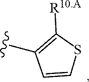

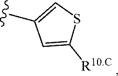

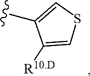

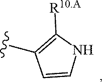

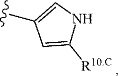

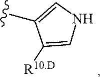

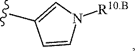

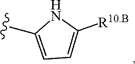

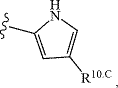

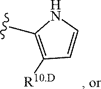

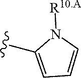

and R^{10.A}, R^{10.B}, R^{10.C}, R^{10.D}, and R^{10.E} are independently hydrogen or any value of R¹⁰ described herein, including in embodiments.

In embodiments, R¹ is independently

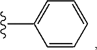

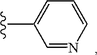

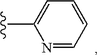

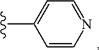

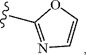

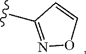

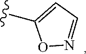

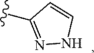

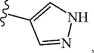

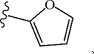

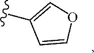

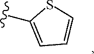

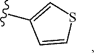

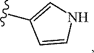

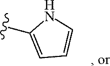

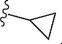

In embodiments, R¹ is independently

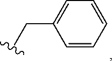

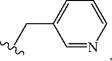

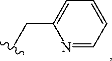

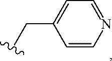

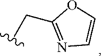

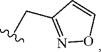

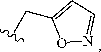

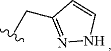

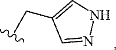

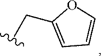

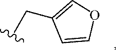

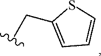

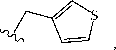

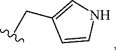

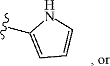

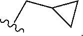

In embodiments, R¹ is independently

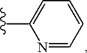

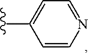

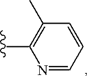

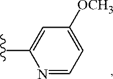

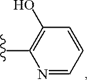

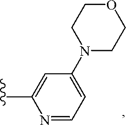

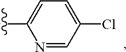

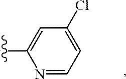

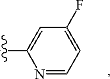

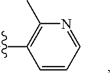

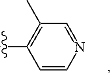

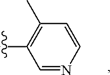

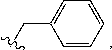

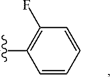

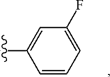

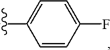

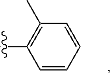

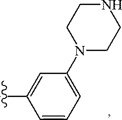

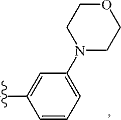

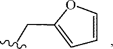

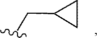

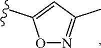

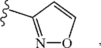

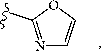

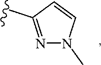

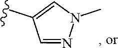

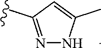

In embodiments, R¹ is independently

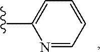

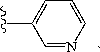

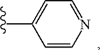

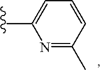

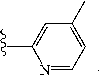

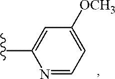

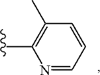

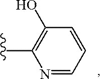

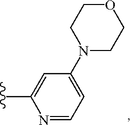

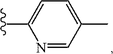

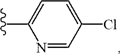

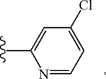

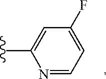

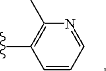

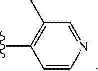

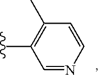

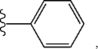

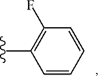

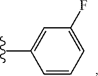

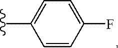

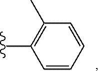

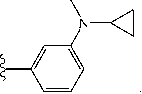

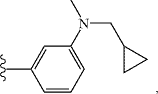

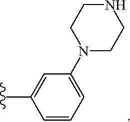

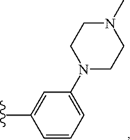

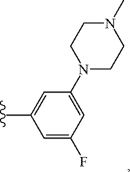

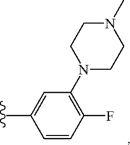

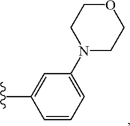

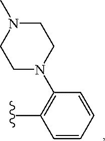

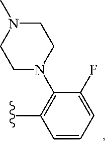

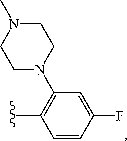

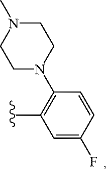

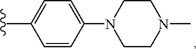

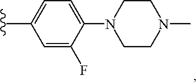

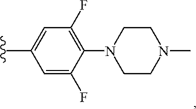

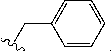

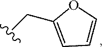

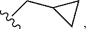

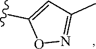

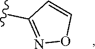

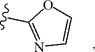

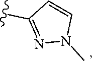

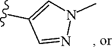

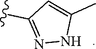

In embodiments, R¹ is independently —SO₂NR^{1A}R^{1B}, —NR^{1A}R^{1B}, or —C(O)NR^{1A}R^{1B}. In embodiments, R¹ is independently —SO₂NR^{1A}R^{1B} or —C(O)NR^{1A}R^{1B}. In embodiments, R¹ is independently —C(O)NR^{1A}R^{1B}.

In embodiments, R¹ is independently —NR^{1A}R^{1B}.

In embodiments, X¹ is independently —F. In embodiments, X¹ is independently —Cl. In embodiments, X¹ is independently —Br. In embodiments, X¹ is independently —I.

In embodiments, n1 is independently 0. In embodiments, n1 is independently 1. In embodiments, n1 is independently 2. In embodiments, n1 is independently 3. In embodiments, n1 is independently 4.

In embodiments, m1 is independently 1. In embodiments, m1 is independently 2. In embodiments, v1 is independently 1. In embodiments, v1 is independently 2.

In embodiments, R^{1A} and R^{1B} are independently hydrogen, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R^{1A} and R^{1B} are independently hydrogen, substituted or unsubstituted alkyl (e.g., C₁—C₈, C₁—C₆, or C₁—C₄), substituted or unsubstituted cycloalkyl (e.g., C₃—C₈, C₃—C₆, or C₅—C₆), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R^{1A} and R^{1B} are independently hydrogen, substituted or unsubstituted C₁—C₆ alkyl, substituted or unsubstituted C₃—C₆ cycloalkyl, substituted or unsubstituted phenyl, or substituted or unsubstituted 5 to 6 membered heteroaryl.

In embodiments, R^{1A} is independently hydrogen. In embodiments, R^{1A} is independently —CCl₃. In embodiments, R^{1A} is independently —CBr₃. In embodiments, R^{1A} is independently —CF₃. In embodiments, R^{1A} is independently —CI₃. In embodiments, R^{1A} is independently —CHCl₂. In embodiments, R^{1A} is independently —CHBr₂. In embodiments, R^{1A} is independently —CHF₂. In embodiments, R^{1A} is independently —CHI2. In embodiments, R^{1A} is independently —CH₂Cl. In embodiments, R^{1A} is independently —CH₂Br. In embodiments, R^{1A} is independently —CH₂F. In embodiments, R^{1A} is independently —CH₂I. In embodiments, R^{1A} is independently —CN. In embodiments, R^{1A} is independently —OH. In embodiments, R^{1A} is independently —NH₂. In embodiments, R^{1A} is independently -COOH. In embodiments, R^{1A} is independently —CONH₂. In embodiments, R^{1A} is independently —OCCl₃. In embodiments, R^{1A} is independently —OCF₃. In embodiments, R^{1A} is independently —OCBr₃. In embodiments, R^{1A} is independently —OCI₃. In embodiments, R^{1A} is independently —OCHCl₂. In embodiments, R^{1A} is independently —OCHBr₂. In embodiments, R^{1A} is independently —OCHI₂. In embodiments, R^{1A} is independently —OCHF₂. In embodiments, R^{1A} is independently —OCH₂Cl. In embodiments, R^{1A} is independently —OCH₂Br. In embodiments, R^{1A} is independently —OCH₂I. In embodiments, R^{1A} is independently —OCH₂F. In embodiments, R^{1A} is independently halogen. In embodiments, R^{1A} is independently —NO₂. In embodiments, R^{1A} is independently —OCH₃. In embodiments, R^{1A} is independently -OCH₂CH₃. In embodiments, R^{1A} is independently —OCH(CH₃)₂. In embodiments, R^{1A} is independently —OC(CH₃)₃. In embodiments, R^{1A} is independently —CH₃. In embodiments, R^{1A} is independently —CH₂CH₃. In embodiments, R^{1A} is independently —CH(CH₃)₂. In embodiments, R^{1A} is independently —C(CH₃)₃. In embodiments, R^{1A} is independently unsubstituted cyclopropyl. In embodiments, R^{1A} is independently unsubstituted cyclobutyl. In embodiments, R^{1A} is independently unsubstituted cyclopentyl. In embodiments, R^{1A} is independently unsubstituted cyclohexyl. In embodiments, R^{1A} is independently substituted or unsubstituted alkyl (e.g., C₁—C₈, C₁—C₆, or C₁—C₄). In embodiments, R^{1A} is independently substituted or unsubstituted heteroalkyl (e.g., 2 to 8 membered, 2 to 6 membered, or 2 to 4 membered). In embodiments, R^{1A} is independently substituted or unsubstituted cycloalkyl (e.g., C₃—C₈, C₃—C₆, or C₅—C₆). In embodiments, R^{1A} is independently substituted or unsubstituted heterocycloalkyl (e.g., 3 to 8 membered, 3 to 6 membered, or 5 to 6 membered). In embodiments, R^{1A} is independently substituted or unsubstituted aryl (e.g., C₆-C₁₀, C₁₀, or phenyl). In embodiments, R^{1A} is independently substituted or unsubstituted heteroaryl (e.g., 5 to 10 membered, 5 to 9 membered, or 5 to 6 membered).

In embodiments, R^{1A} is independently hydrogen, unsubstituted C₁—C₄ alkyl, or unsubstituted cyclopropyl.

In embodiments, R^{1A} are independently substituted or unsubstituted phenyl or substituted or unsubstituted 5 to 6 membered heteroaryl. In embodiments, R^{1A} is independently R¹⁰-substituted phenyl or R¹⁰-substituted 5 to 6 membered heteroaryl. In embodiments, R^{1A} is independently

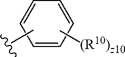

and R¹⁰ is as described herein and z10 is independently an integer from 0 to 5. In embodiments, R^{1A} is independently

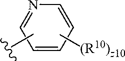

and R¹⁰ is as described herein and z10 is independently an integer from 0 to 4. In embodiments, R^{1A} is independently

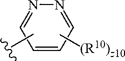

and R¹⁰ is as described herein and z10 is independently an integer from 0 to 3. In embodiments, R^{1A} is independently

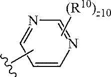

and R¹⁰ is as described herein and z10 is independently an integer from 0 to 3. In embodiments, R^{1A} is independently

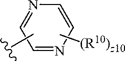

and R¹⁰ is as described herein and z10 is independently an integer from 0 to 3.

In embodiments, R^{1A} is independently

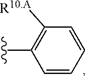

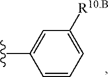

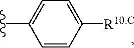

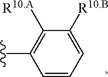

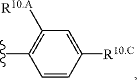

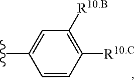

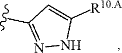

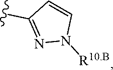

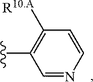

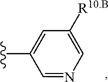

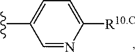

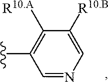

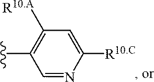

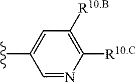

and R^{10.A}, R^{10.B}, and R^{10.C} are independently hydrogen or any value of R¹⁰ described herein, including in embodiments.

In embodiments, R^{1A} is independently

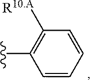

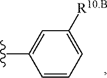

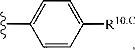

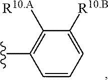

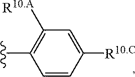

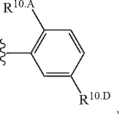

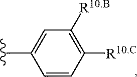

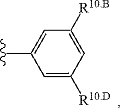

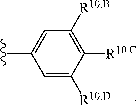

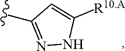

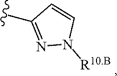

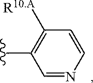

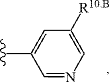

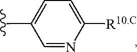

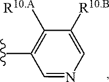

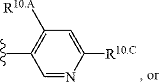

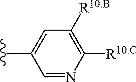

and R^{10.A}, R^{10.B}, R¹⁰^{.C}, and R^{10.D} are independently hydrogen or any value of R¹⁰ described herein, including in embodiments.

In embodiments, R^{1A} is independently

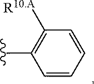

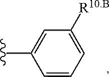

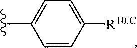

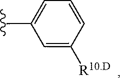

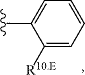

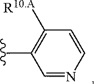

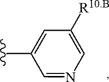

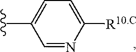

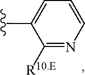

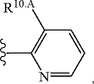

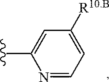

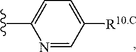

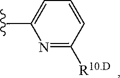

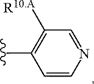

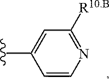

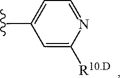

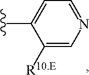

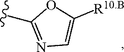

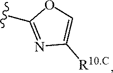

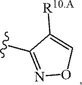

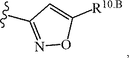

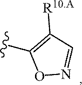

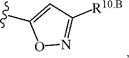

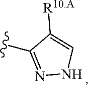

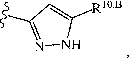

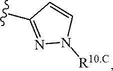

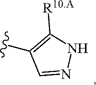

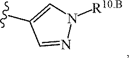

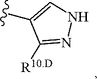

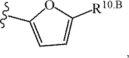

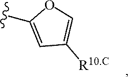

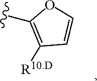

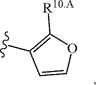

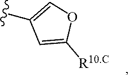

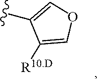

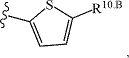

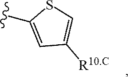

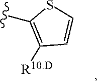

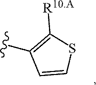

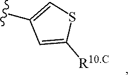

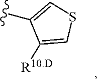

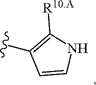

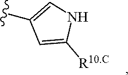

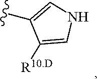

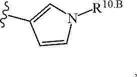

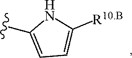

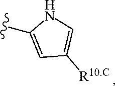

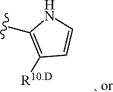

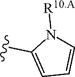

and R^{10.A}, R^{10.B}, R^{10.C}, R^{10.D}, and R^{10.E} are independently hydrogen or any value of R¹⁰ described herein, including in embodiments.

In embodiments, R^{1A} is independently

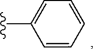

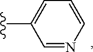

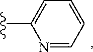

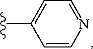

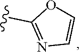

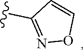

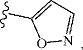

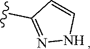

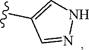

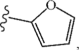

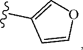

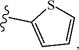

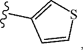

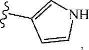

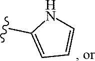

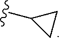

In embodiments, R^{1A} is independently

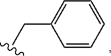

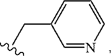

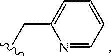

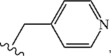

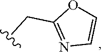

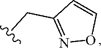

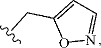

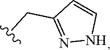

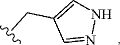

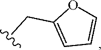

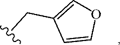

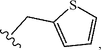

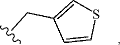

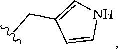

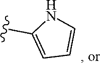

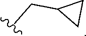

In embodiments, R^{1A} is independently

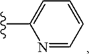

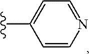

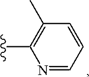

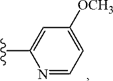

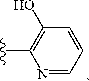

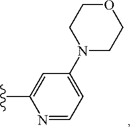

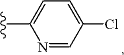

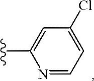

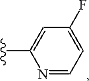

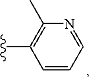

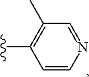

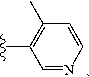

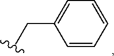

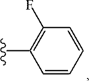

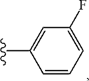

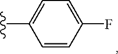

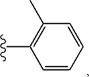

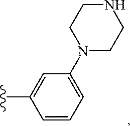

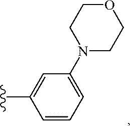

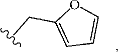

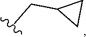

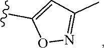

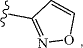

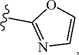

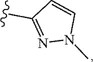

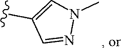

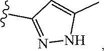

In embodiments, R^{1A} is independently

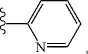

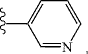

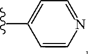

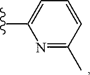

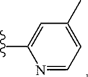

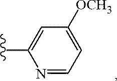

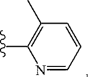

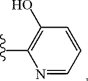

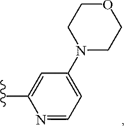

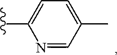

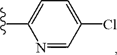

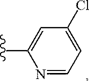

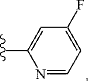

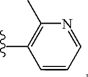

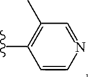

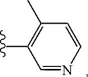

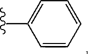

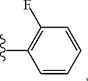

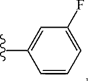

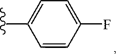

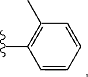

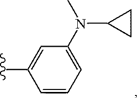

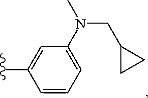

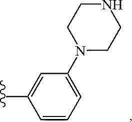

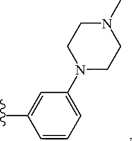

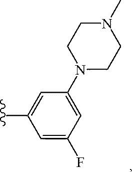

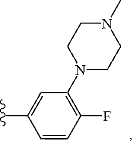

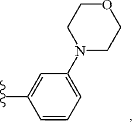

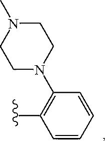

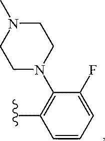

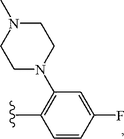

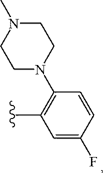

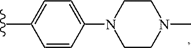

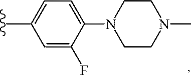

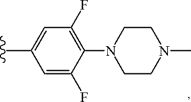

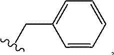

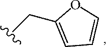

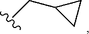

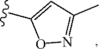

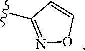

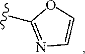

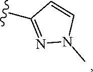

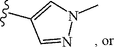

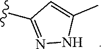

In embodiments, R^{1B} is independently hydrogen. In embodiments, R^{1B} is independently —CCl₃. In embodiments, R^{1B} is independently —CBr₃. In embodiments, R^{1B} is independently —CF₃. In embodiments, R^{1B} is independently —CI₃. In embodiments, R^{1B} is independently —CHCl₂. In embodiments, R^{1B} is independently —CHBr₂. In embodiments, R^{1B} is independently —CHF₂. In embodiments, R^{1B} is independently —CHI2. In embodiments, R^{1B} is independently —CH₂Cl. In embodiments, R^{1B} is independently —CH2Br. In embodiments, R^{1B} is independently —CH₂F. In embodiments, R^{1B} is independently —CH₂I. In embodiments, R^{1B} is independently —CN. In embodiments, R^{1B} is independently —OH. In embodiments, R^{1B} is independently —NH₂. In embodiments, R^{1B} is independently —COOH. In embodiments, R^{1B} is independently —CONH₂. In embodiments, R^{1B} is independently —OCCl₃. In embodiments, R^{1B} is independently —OCF₃. In embodiments, R^{1B} is independently —OCBr₃. In embodiments, R^{1B} is independently —OCI₃. In embodiments, R^{1B} is independently —OCHCl₂. In embodiments, R^{1B} is independently —OCHBr₂. In embodiments, R^{1B} is independently —OCHI2. In embodiments, R^{1B} is independently —OCHF₂. In embodiments, R^{1B} is independently —OCH₂Cl. In embodiments, R^{1B} is independently —OCH₂Br. In embodiments, R^{1B} is independently —OCH₂I. In embodiments, R^{1B} is independently —OCH₂F. In embodiments, R^{1B} is independently halogen. In embodiments, R^{1B} is independently —NO₂. In embodiments, R^{1B} is independently —OCH₃. In embodiments, R^{1B} is independently —OCH₂CH₃. In embodiments, R^{1B} is independently —OCH(CH₃)₂. In embodiments, R^{1B} is independently —OC(CH₃)₃. In embodiments, R^{1B} is independently —CH₃. In embodiments, R^{1B} is independently —CH₂CH₃. In embodiments, R^{1B} is independently —CH(CH₃)₂. In embodiments, R^{1B} is independently —C(CH₃)₃. In embodiments, R^{1B} is independently unsubstituted cyclopropyl. In embodiments, R^{1B} is independently unsubstituted cyclobutyl. In embodiments, R^{1B} is independently unsubstituted cyclopentyl. In embodiments, R^{1B} is independently unsubstituted cyclohexyl. In embodiments, R^{1B} is independently substituted or unsubstituted alkyl (e.g., C₁—C₈, C₁—C₆, or C₁—C₄). In embodiments, R^{1B} is independently substituted or unsubstituted heteroalkyl (e.g., 2 to 8 membered, 2 to 6 membered, or 2 to 4 membered). In embodiments, R^{1B} is independently substituted or unsubstituted cycloalkyl (e.g., C₃—C₈, C₃—C₆, or C₅—C₆). In embodiments, R^{1B} is independently substituted or unsubstituted heterocycloalkyl (e.g., 3 to 8 membered, 3 to 6 membered, or 5 to 6 membered). In embodiments, R^{1B} is independently substituted or unsubstituted aryl (e.g., C₆-C₁₀, C₁₀, or phenyl). In embodiments, R^{1B} is independently substituted or unsubstituted heteroaryl (e.g., 5 to 10 membered, 5 to 9 membered, or 5 to 6 membered).

In embodiments, R^{1B} is independently hydrogen, unsubstituted C₁—C₄ alkyl, or unsubstituted cyclopropyl.

In embodiments, R^{1B} are independently substituted or unsubstituted phenyl or substituted or unsubstituted 5 to 6 membered heteroaryl. In embodiments, R^{1B} is independently R¹⁰-substituted phenyl or R¹⁰-substituted 5 to 6 membered heteroaryl. In embodiments, R^{1B} is independently

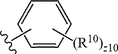

and R¹⁰ is as described herein and z10 is independently an integer from 0 to 5. In embodiments, R^{1B} is independently

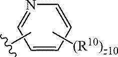

and R¹⁰ is as described herein and z10 is independently an integer from 0 to 4. In embodiments, R^{1B} is independently

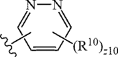

and R¹⁰ is as described herein and z10 is independently an integer from 0 to 3. In embodiments, R^{1B} is independently

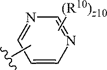

and R¹⁰ is as described herein and z10 is independently an integer from 0 to 3. In embodiments, R^{1B} is independently

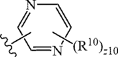

and R¹⁰ is as described herein and z10 is independently an integer from 0 to 3.

In embodiments, R^{1B} is independently

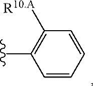

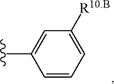

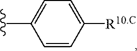

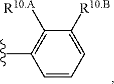

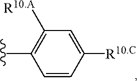

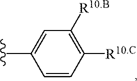

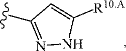

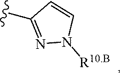

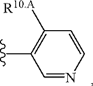

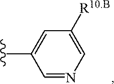

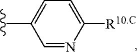

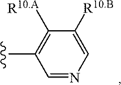

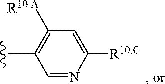

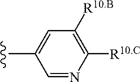

and R^{10.A}, R^{10.B}, and R^{10.C} are independently hydrogen or any value of R¹⁰ described herein, including in embodiments.

In embodiments, R^{1B} is independently

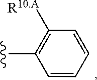

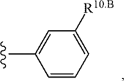

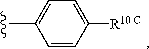

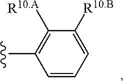

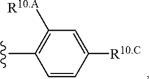

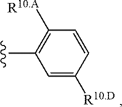

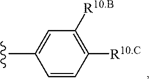

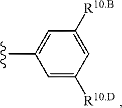

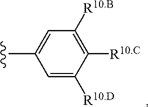

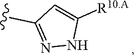

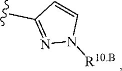

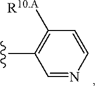

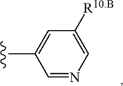

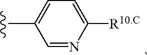

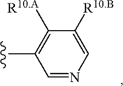

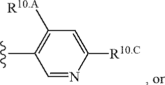

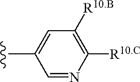

and R^{10.A}, R^{10.B} , R^{10.C}, and R^{10.D} are independently hydrogen or any value of R¹⁰ described herein, including in embodiments.

In embodiments, R^{1B} is independently

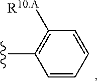

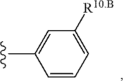

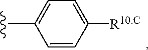

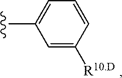

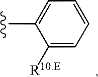

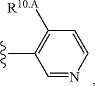

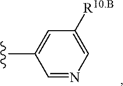

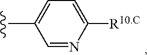

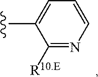

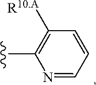

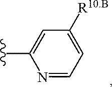

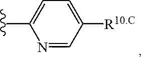

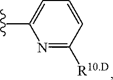

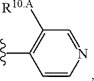

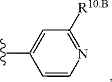

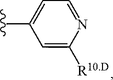

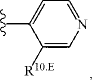

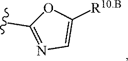

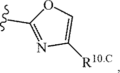

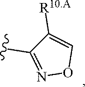

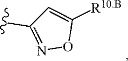

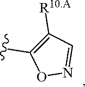

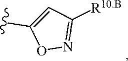

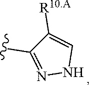

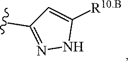

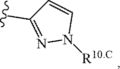

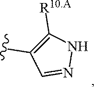

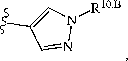

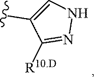

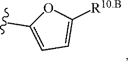

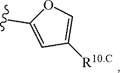

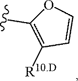

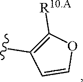

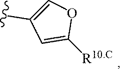

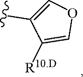

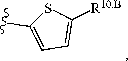

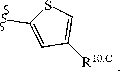

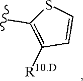

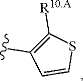

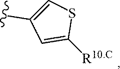

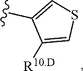

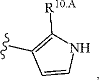

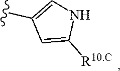

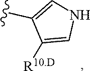

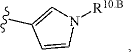

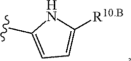

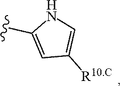

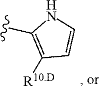

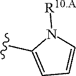

and R^{10.A}, R^{10.B}, R^{10.C}, R^{10.D}, and R^{10.E} are independently hydrogen or any value of R¹⁰ described herein, including in embodiments..

In embodiments, R^{1B} is independently

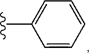

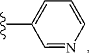

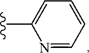

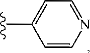

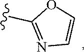

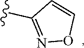

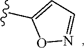

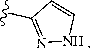

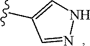

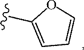

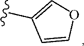

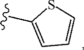

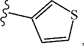

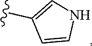

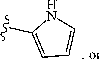

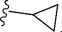

In embodiments, R^{1B} is independently

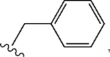

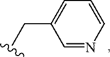

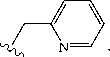

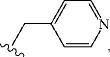

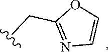

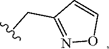

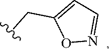

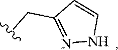

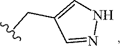

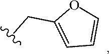

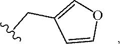

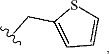

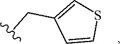

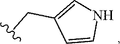

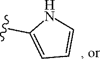

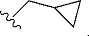

In embodiments, R^{1B} is independently

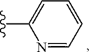

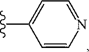

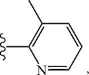

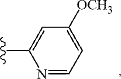

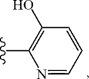

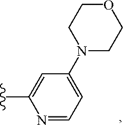

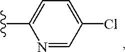

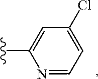

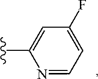

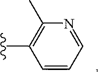

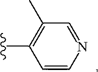

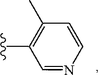

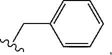

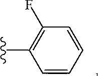

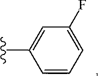

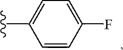

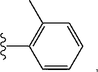

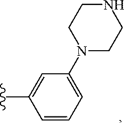

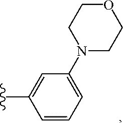

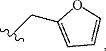

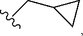

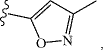

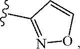

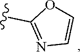

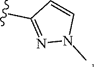

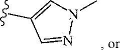

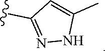

In embodiments, R^{1B} is independently

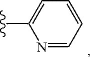

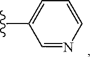

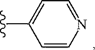

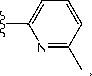

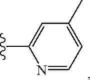

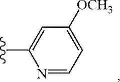

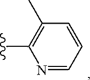

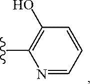

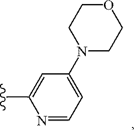

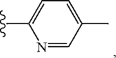

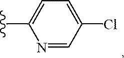

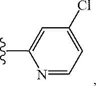

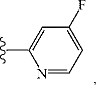

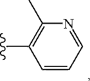

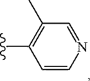

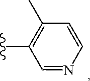

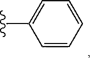

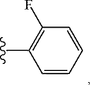

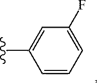

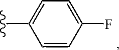

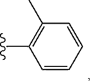

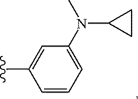

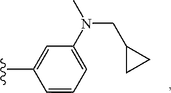

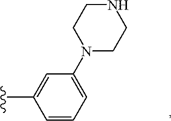

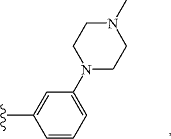

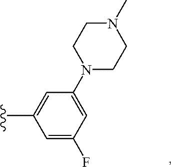

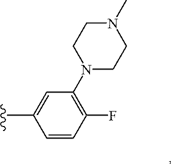

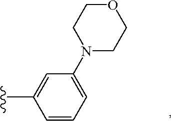

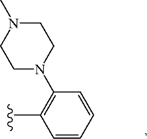

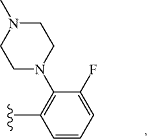

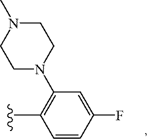

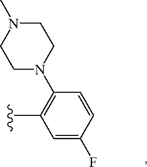

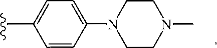

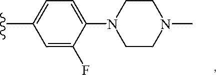

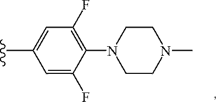

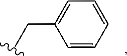

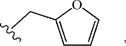

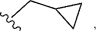

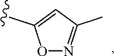

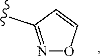

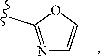

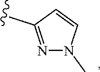

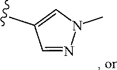

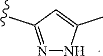

In embodiments, R^{1A} and R^{1B} substituents bonded to the same nitrogen atom are joined to form a substituted or unsubstituted heterocycloalkyl (e.g., 3 to 8 membered, 3 to 6 membered, or 5 to 6 membered). In embodiments, R^{1A} and R^{1B} substituents bonded to the same nitrogen atom are joined to form a substituted or unsubstituted C₃—C₆ heterocycloalkyl. In embodiments, R^{1A} and R^{1B} bonded to the same nitrogen atom are joined to form a substituted or unsubstituted piperazinyl. In embodiments, R^{1A} and R^{1B} substituents bonded to the same nitrogen atom are joined to form a substituted or unsubstituted heteroaryl (e.g., 5 to 10 membered, 5 to 9 membered, or 5 to 6 membered). In embodiments, R^{1A} and R^{1B} substituents bonded to the same nitrogen atom are joined to form a substituted or unsubstituted 5 to 6 membered heteroaryl. In embodiments, R^{1A} and R^{1B} bonded to the same nitrogen atom are joined to form

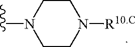

In embodiments, R^{1A} and R^{1B} bonded to the same nitrogen atom are joined to form

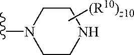

wherein R¹⁰ and z10 are as described herein. In embodiments, R^{1A} and R^{1B} bonded to the same nitrogen atom are joined to form

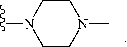

In embodiments, R^{1C} is independently hydrogen. In embodiments, R^{1C} is independently —CCl₃. In embodiments, R^{1C} is independently —CBr₃. In embodiments, R^{1C} is independently —CF₃. In embodiments, R^{1C} is independently —CI₃. In embodiments, R^{1C} is independently —CHCl₂. In embodiments, R^{1C} is independently —CHBr₂. In embodiments, R^{1C} is independently —CHF₂. In embodiments, R^{1C} is independently —CHI2. In embodiments, R^{1C} is independently —CH₂Cl. In embodiments, R^{1C} is independently —CH₂Br. In embodiments, R^{1C} is independently —CH₂F. In embodiments, R^{1C} is independently —CH₂I. In embodiments, R^{1C} is independently —CN. In embodiments, R^{1C} is independently —OH. In embodiments, R^{1C} is independently —NH₂. In embodiments, R^{1C} is independently —COOH. In embodiments, R^{1C} is independently —CONH₂. In embodiments, R^{1C} is independently —OCCl₃. In embodiments, R^{1C} is independently —OCF₃. In embodiments, R^{1C} is independently —OCBr₃. In embodiments, R^{1C} is independently —OCI₃. In embodiments, R^{1C} is independently —OCHCl₂. In embodiments, R^{1C} is independently —OCHBr₂. In embodiments, R^{1C} is independently —OCHI2. In embodiments, R^{1C} is independently —OCHF₂. In embodiments, R^{1C} is independently —OCH₂Cl. In embodiments, R^{1C} is independently —OCH₂Br. In embodiments, R^{1C} is independently —OCH₂I. In embodiments, R^{1C} is independently —OCH₂F. In embodiments, R^{1C} is independently halogen. In embodiments, R^{1C} is independently —NO₂. In embodiments, R^{1C} is independently —OCH₃. In embodiments, R^{1C} is independently -OCH₂CH₃. In embodiments, R^{1C} is independently —OCH(CH₃)₂. In embodiments, R^{1C} is independently-OC(CH₃)₃. In embodiments, R^{1C} is independently —CH₃. In embodiments, R^{1C} is independently —CH₂CH₃. In embodiments, R^{1C} is independently —CH(CH₃)₂. In embodiments, R^{1C} is independently —C(CH₃)₃. In embodiments, R^{1C} is independently unsubstituted cyclopropyl. In embodiments, R^{1C} is independently unsubstituted cyclobutyl. In embodiments, R^{1C} is independently unsubstituted cyclopentyl. In embodiments, R^{1C} is independently unsubstituted cyclohexyl. In embodiments, R^{1C} is independently substituted or unsubstituted alkyl (e.g., C₁—C₈, C₁—C₆, or C₁—C₄). In embodiments, R^{1C} is independently substituted or unsubstituted heteroalkyl (e.g., 2 to 8 membered, 2 to 6 membered, or 2 to 4 membered). In embodiments, R^{1C} is independently substituted or unsubstituted cycloalkyl (e.g., C₃—C₈, C₃—C₆, or C₅—C₆). In embodiments, R^{1C} is independently substituted or unsubstituted heterocycloalkyl (e.g., 3 to 8 membered, 3 to 6 membered, or 5 to 6 membered). In embodiments, R^{1C} is independently substituted or unsubstituted aryl (e.g., C₆-C₁₀, C₁₀, or phenyl). In embodiments, R^{1C} is independently substituted or unsubstituted heteroaryl (e.g., 5 to 10 membered, 5 to 9 membered, or 5 to 6 membered).

In embodiments, R^{1D} is independently hydrogen. In embodiments, R^{1D} is independently —CCl₃. In embodiments, R^{1D} is independently —CBr₃. In embodiments, R^{1D} is independently —CF₃. In embodiments, R^{1D} is independently —CI₃. In embodiments, R^{1D} is independently —CHCl₂. In embodiments, R^{1D} is independently —CHBr₂. In embodiments, R^{1D} is independently —CHF₂. In embodiments, R^{1D} is independently —CHI2. In embodiments, R^{1D} is independently —CH₂Cl. In embodiments, R^{1D} is independently —CH₂Br. In embodiments, R^{1D} is independently —CH₂F. In embodiments, R^{1D} is independently —CH₂I. In embodiments, R^{1D} is independently —CN. In embodiments, R^{1D} is independently —OH. In embodiments, R^{1D} is independently —NH₂. In embodiments, R^{1D} is independently -COOH. In embodiments, R^{1D} is independently —CONH₂. In embodiments, R^{1D} is independently —OCCl₃. In embodiments, R^{1D} is independently —OCF₃. In embodiments, R^{1D} is independently —OCBr₃. In embodiments, R^{1D} is independently —OCI₃. In embodiments, R^{1D} is independently —OCHCl₂. In embodiments, R^{1D} is independently —OCHBr₂. In embodiments, R^{1D} is independently —OCHI2. In embodiments, R^{1D} is independently —OCHF₂. In embodiments, R^{1D} is independently —OCH₂Cl. In embodiments, R^{1D} is independently —OCH₂Br. In embodiments, R^{1D} is independently —OCH₂I. In embodiments, R^{1D} is independently —OCH₂F. In embodiments, R^{1D} is independently halogen. In embodiments, R^{1D} is independently —NO₂. In embodiments, R^{1D} is independently —OCH₃. In embodiments, R^{1D} is independently -OCH₂CH₃. In embodiments, R^{1D} is independently —OCH(CH₃)₂. In embodiments, R^{1D} is independently —OC(CH₃)₃. In embodiments, R^{1D} is independently —CH₃. In embodiments, R^{1D} is independently —CH₂CH₃. In embodiments, R^{1D} is independently —CH(CH₃)₂. In embodiments, R^{1D} is independently —C(CH₃)₃. In embodiments, R^{1D} is independently unsubstituted cyclopropyl. In embodiments, R^{1D} is independently unsubstituted cyclobutyl. In embodiments, R^{1D} is independently unsubstituted cyclopentyl. In embodiments, R^{1D} is independently unsubstituted cyclohexyl. In embodiments, R^{1D} is independently substituted or unsubstituted alkyl (e.g., C₁—C₈, C₁—C₆, or C₁—C₄). In embodiments, R^{1D} is independently substituted or unsubstituted heteroalkyl (e.g., 2 to 8 membered, 2 to 6 membered, or 2 to 4 membered). In embodiments, R^{1D} is independently substituted or unsubstituted cycloalkyl (e.g., C₃—C₈, C₃—C₆, or C₅—C₆). In embodiments, R^{1D} is independently substituted or unsubstituted heterocycloalkyl (e.g., 3 to 8 membered, 3 to 6 membered, or 5 to 6 membered). In embodiments, R^{1D} is independently substituted or unsubstituted aryl (e.g., C₆-C₁₀, C₁₀, or phenyl). In embodiments, R^{1D} is independently substituted or unsubstituted heteroaryl (e.g., 5 to 10 membered, 5 to 9 membered, or 5 to 6 membered).

R¹⁰ is independently oxo, halogen, -CX¹⁰₃, -CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR¹⁰CNR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR¹⁰AC(O)OR^{10C}, —NR^{10A}OR^{10C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); two adjacent R¹⁰ substituents may optionally be joined to form a substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

X¹⁰ is independently —F, —Cl, —Br, or —I. In embodiments, X¹⁰ is independently —F. In embodiments, X¹⁰ is independently —Cl. In embodiments, X¹⁰ is independently —Br. In embodiments, X¹⁰ is independently —I.

n10 is independently an integer from 0 to 4. In embodiments, n10 is independently 0. In embodiments, n10 is independently 1. In embodiments, n10 is independently 2. In embodiments, n10 is independently 3. In embodiments, n10 is independently 4.

m10 and v10 are independently 1 or 2. In embodiments, m10 is independently 1. In embodiments, m10 is independently 2. In embodiments, v10 is independently 1. In embodiments, v10 is independently 2.

In embodiments, R¹⁰ is independently halogen, -CX¹⁰₃, -CHX¹⁰₂, —CH₂X¹⁰, —OCX¹⁰₃, —OCH₂X¹⁰, —OCHX¹⁰₂, —CN, —SOₙ₁₀R^{10D}, —SOᵥ₁₀₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR1^{0A}C(O)R^{10C}, —NR^{10A}C(O)OR^{10C}, —NR^{10A}OR^{10C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); two adjacent R¹⁰ substituents may optionally be joined to form a substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered). In embodiments, R¹⁰ is independently halogen, —CX¹⁰₃, —CHX¹⁰₂, —CH₂X¹⁰, —OCX¹⁰₃, —OCH₂X¹⁰, —OCHX¹⁰₂, —CN, —SO₂R^{10D}, —SR^{1OD}, —C(O)R^{10C}, —OR^{10D}, substituted or unsubstituted C₁—C₆ alkyl, substituted or unsubstituted 2 to 6 membered heteroalkyl, substituted or unsubstituted C₃—C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R¹⁰ is independently halogen, —CX¹⁰₃, -CHX¹⁰₂, —CH₂X¹⁰, —OCX¹⁰₃, —OCH₂X¹⁰, —OCHX¹⁰₂, —SR^{10D}, —OR^{10D}, unsubstituted C₁—C₄ alkyl, unsubstituted 2 to 4 membered heteroalkyl, unsubstituted C₃—C₆ cycloalkyl, unsubstituted 3 to 6 membered heterocycloalkyl, unsubstituted phenyl, or unsubstituted 5 to 6 membered heteroaryl.

In embodiments, R¹⁰ is independently halogen, —OH, —OCH₃, —CH₃, unsubstituted 6 membered heterocycloalkyl. In embodiments, R¹⁰ is independently halogen. In embodiments, R¹⁰ is independently —OH. In embodiments, R¹⁰ is independently —OCH₃. In embodiments, R¹⁰ is independently —CH₃. In embodiments, R¹⁰ is independently unsubstituted 6 membered heterocycloalkyl. In embodiments, R¹⁰ is independently —F. In embodiments, R¹⁰ is independently —Cl. In embodiments, R¹⁰ is independently unsubstituted morpholinyl. In embodiments, R¹⁰ is independently unsubstituted piperazinyl. In embodiments, R¹⁰ is independently substituted piperazinyl. In embodiments, R¹⁰ is independently

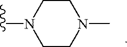

In embodiments, R¹⁰ is independently unsubstituted furanyl.

In embodiments, R¹⁰ is independently substituted or unsubstituted C₁—C₆ alkyl. In embodiments, R¹⁰ is independently substituted or unsubstituted 2 to 6 membered heteroalkyl. In embodiments, R¹⁰ is independently substituted or unsubstituted C₃—C₆ cycloalkyl. In embodiments, R¹⁰ is independently substituted or unsubstituted 3 to 6 membered heterocycloalkyl. In embodiments, R¹⁰ is independently substituted or unsubstituted phenyl. In embodiments, R¹⁰ is independently substituted or unsubstituted 5 to 6 membered heteroaryl.

In embodiments, R¹⁰ is independently oxo, halogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHC1₂, —OCHBr₂, —OCHI₂, —OCHF₂, —OCH₂Cl, —OCH₂Br, —OCH₂I, —OCH₂F, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R¹⁰ is independently halogen. In embodiments, R¹⁰ is independently —F. In embodiments, R¹⁰ is independently —Cl. In embodiments, R¹⁰ is independently —Br. In embodiments, R¹⁰ is independently —I. In embodiments, R¹⁰ is independently oxo. In embodiments, R¹⁰ is independently —CX¹⁰₃. In embodiments, R¹⁰ is independently -CHX¹⁰₂. In embodiments, R¹⁰ is independently -CH₂X¹^{°}. In embodiments, R¹⁰ is independently —OCX¹⁰₃. In embodiments, R¹⁰ is independently —OCH₂X¹⁰. In embodiments, R¹⁰ is independently -OCHX¹⁰₂. In embodiments, R¹⁰ is independently —CN. In embodiments, R¹⁰ is independently -SOₙ₁₀R^{10D}. In embodiments, R¹⁰ is independently -SOᵥ₁₀NR^{10A}R^{10B}. In embodiments, R¹⁰ is independently —NR^{10C}NR^{10A}R^{10B}. In embodiments, R¹⁰ is independently -ONR^{10A}R^{10B}. In embodiments, R¹⁰ is independently —NHC(O)NR¹⁰CNR^{10A}R^{10B}. In embodiments, R¹⁰ is independently —NHC(O)NR^{10A}R^{10B}. In embodiments, R¹⁰ is independently —N(O)ₘ₁₀. In embodiments, R¹⁰ is independently —NR^{10A}R^{10B}. In embodiments, R¹⁰ is independently —C(O)R^{10C}. In embodiments, R¹⁰ is independently —C(O)—OR^{10C}. In embodiments, R¹⁰ is independently —C(O)NR^{10A}R^{10B}. In embodiments, R¹⁰ is independently -OR^{10D}. In embodiments, R¹⁰ is independently -NR^{10A}SO₂R^{10D}. In embodiments, R¹⁰ is independently —NR^{10A}C(O)R^{10C}. In embodiments, R¹⁰ is independently —NR^{10A}C(O)OR^{10C}. In embodiments, R¹⁰ is independently —NR^{10A}OR^{10C}. In embodiments, R¹⁰ is independently —SF₅. In embodiments, R¹⁰ is independently —N₃.

In embodiments, R¹⁰ is independently —NR^{10A}R^{10B}. In embodiments, R^{10A} is independently substituted or unsubstituted C₁—C₄ alkyl. In embodiments, R^{10A} is independently unsubstituted C₁—C₄ alkyl. In embodiments, R^{10A} is independently substituted methyl. In embodiments, R^{10A} is independently unsubstituted methyl. In embodiments, R^{10A} is independently substituted ethyl. In embodiments, R^{10A} is independently unsubstituted ethyl. In embodiments, R^{10A} is independently substituted propyl. In embodiments, R^{10A} is independently unsubstituted propyl. In embodiments, R^{10A} is independently substituted butyl. In embodiments, R^{10A} is independently unsubstituted butyl. In embodiments, R^{10A} is independently substituted or unsubstituted C₃—C₆ cycloalkyl. In embodiments, R^{10A} is independently unsubstituted C₃—C₆ cycloalkyl. In embodiments, R^{10A} is independently unsubstituted cyclopropyl. In embodiments, R^{10A} is independently unsubstituted cyclobutyl. In embodiments, R^{10A} is independently unsubstituted cyclopentyl. In embodiments, R^{10A} is independently unsubstituted cyclohexyl. In embodiments, R^{10B} is independently substituted or unsubstituted C₁—C₄ alkyl. In embodiments, R^{10B} is independently unsubstituted C₁—C₄ alkyl. In embodiments, R^{10B} is independently substituted methyl. In embodiments, R^{10B} is independently unsubstituted methyl. In embodiments, R^{10B} is independently substituted ethyl. In embodiments, R^{10B} is independently unsubstituted ethyl. In embodiments, R^{10B} is independently substituted propyl. In embodiments, R^{10B} is independently unsubstituted propyl. In embodiments, R^{10B} is independently substituted butyl. In embodiments, R^{10B} is independently unsubstituted butyl. In embodiments, R^{10B} is independently substituted or unsubstituted C₃—C₆ cycloalkyl. In embodiments, R^{10B} is independently unsubstituted C₃—C₆ cycloalkyl. In embodiments, R^{10B} is independently unsubstituted cyclopropyl. In embodiments, R^{10B} is independently unsubstituted cyclobutyl. In embodiments, R^{10B} is independently unsubstituted cyclopentyl. In embodiments, R^{10B} is independently unsubstituted cyclohexyl. In embodiments, R¹⁰ is independently

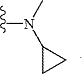

In embodiments, R¹⁰ is independently

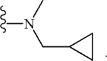

In embodiments, R¹⁰ is independently —SCH₃. In embodiments, R¹⁰ is independently —OCH₃. In embodiments, R¹⁰ is independently unsubstituted C₁—C₄ alkyl. In embodiments, R¹⁰ is independently unsubstituted cyclopropyl. In embodiments, R¹⁰ is independently unsubstituted phenyl. In embodiments, R¹⁰ is independently hydrogen. In embodiments, R¹⁰ is independently —CCl₃. In embodiments, R¹⁰ is independently —CBr₃. In embodiments, R¹⁰ is independently —CF₃. In embodiments, R¹⁰ is independently —CI₃. In embodiments, R¹⁰ is independently —CHCl₂. In embodiments, R¹⁰ is independently —CHBr₂. In embodiments, R¹⁰ is independently —CHF₂. In embodiments, R¹⁰ is independently —CHI2. In embodiments, R¹⁰ is independently —CH₂Cl. In embodiments, R¹⁰ is independently -CH₂Br. In embodiments, R¹⁰ is independently —CH₂F. In embodiments, R¹⁰ is independently —CH₂I. In embodiments, R¹⁰ is independently —CN. In embodiments, R¹⁰ is independently —OH. In embodiments, R¹⁰ is independently —NH₂. In embodiments, R¹⁰ is independently —COOH. In embodiments, R¹⁰ is independently —CONH₂. In embodiments, R¹⁰ is independently —OCCl₃. In embodiments, R¹⁰ is independently —OCF₃. In embodiments, R¹⁰ is independently —OCBr₃. In embodiments, R¹⁰ is independently —OCI₃. In embodiments, R¹⁰ is independently —OCHCl₂. In embodiments, R¹⁰ is independently -OCHBr₂. In embodiments, R¹⁰ is independently —OCHI2. In embodiments, R¹⁰ is independently —OCHF₂. In embodiments, R¹⁰ is independently —OCH₂Cl. In embodiments, R¹⁰ is independently —OCH2Br. In embodiments, R¹⁰ is independently —OCH₂I. In embodiments, R¹⁰ is independently —OCH₂F. In embodiments, R¹⁰ is independently halogen. In embodiments, R¹⁰ is independently —NO₂. In embodiments, R¹⁰ is independently —OCH₃. In embodiments, R¹⁰ is independently —OCH₂CH₃. In embodiments, R¹⁰ is independently -OCH(CH₃)₂. In embodiments, R¹⁰ is independently —OC(CH₃)₃. In embodiments, R¹⁰ is independently —CH₃. In embodiments, R¹⁰ is independently —CH₂CH₃. In embodiments, R¹⁰ is independently —CH(CH₃)₂. In embodiments, R¹⁰ is independently —C(CH₃)₃. In embodiments, R¹⁰ is independently unsubstituted cyclopropyl. In embodiments, R¹⁰ is independently unsubstituted cyclobutyl. In embodiments, R¹⁰ is independently unsubstituted cyclopentyl. In embodiments, R¹⁰ is independently unsubstituted cyclohexyl. In embodiments, R¹⁰ is independently substituted or unsubstituted alkyl (e.g., C₁—C₈, C₁—C₆, or C₁—C₄). In embodiments, R¹⁰ is independently substituted or unsubstituted heteroalkyl (e.g., 2 to 8 membered, 2 to 6 membered, or 2 to 4 membered). In embodiments, R¹⁰ is independently substituted or unsubstituted cycloalkyl (e.g., C₃—C₈, C₃—C₆, or C₅—C₆). In embodiments, R¹⁰ is independently substituted or unsubstituted heterocycloalkyl (e.g., 3 to 8 membered, 3 to 6 membered, or 5 to 6 membered). In embodiments, R¹⁰ is independently substituted or unsubstituted aryl (e.g., C₆—C₁₀, C₁₀, or phenyl). In embodiments, R¹⁰ is independently substituted or unsubstituted heteroaryl (e.g., 5 to 10 membered, 5 to 9 membered, or 5 to 6 membered). In embodiments, R¹⁰ is independently unsubstituted alkyl (e.g., C₁—C₈, C₁—C₆, or C₁—C₄). In embodiments, R¹⁰ is independently unsubstituted heteroalkyl (e.g., 2 to 8 membered, 2 to 6 membered, or 2 to 4 membered). In embodiments, R¹⁰ is independently unsubstituted cycloalkyl (e.g., C₃—C₈, C₃—C₆, or C₅—C₆). In embodiments, R¹⁰ is independently unsubstituted heterocycloalkyl (e.g., 3 to 8 membered, 3 to 6 membered, or 5 to 6 membered). In embodiments, R¹⁰ is independently unsubstituted aryl (e.g., C₆—C₁₀, C₁₀, or phenyl). In embodiments, R¹⁰ is independently unsubstituted heteroaryl (e.g., 5 to 10 membered, 5 to 9 membered, or 5 to 6 membered).

In embodiments, two adjacent R¹⁰ substituents are joined to form a substituted or unsubstituted cycloalkyl (e.g., C₃—C₈, C₃—C₆, or C₅—C₆). In embodiments, two adjacent R¹⁰ substituents are joined to form a substituted or unsubstituted heterocycloalkyl (e.g., 3 to 8 membered, 3 to 6 membered, or 5 to 6 membered). In embodiments, two adjacent R¹⁰ substituents are joined to form a substituted or unsubstituted aryl (e.g., C₆—C₁₀, C₁₀, or phenyl). In embodiments, two adjacent R¹⁰ substituents are joined to form a substituted or unsubstituted heteroaryl (e.g., 5 to 10 membered, 5 to 9 membered, or 5 to 6 membered). In embodiments, two adjacent R¹⁰ substituents are joined to form an unsubstituted cycloalkyl (e.g., C₃—C₈, C₃—C₆, or C₅—C₆). In embodiments, two adjacent R¹⁰ substituents are joined to form an unsubstituted heterocycloalkyl (e.g., 3 to 8 membered, 3 to 6 membered, or 5 to 6 membered). In embodiments, two adjacent R¹⁰ substituents are joined to form an unsubstituted aryl (e.g., C₆—C₁₀, C₁₀, or phenyl). In embodiments, two adjacent R¹⁰ substituents are joined to form an unsubstituted heteroaryl (e.g., 5 to 10 membered, 5 to 9 membered, or 5 to 6 membered).

R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); R^{10A} and R^{10B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

In embodiments, R^{10A}, R^{10B}, R^{10C}, and R^{10D} are independently hydrogen, —CCl₃, —CBr₃, —CF₃, —CI₃,—CHCl₂, —CHBr₂, —CHF₂, —CHI₂, —CH₂Cl, —CH₂Br, —CH₂F, —CH₂I, unsubstituted C₁—C₆ alkyl, or unsubstituted C₃—C₆ cycloalkyl. In embodiments, R^{10A}, R^{10B}, R^{10C}, and R^{10D} are independently hydrogen, —CCl₃, —CBr₃, —CF₃, —CI₃,—CHCl₂, —CHBr₂, —CHF₂, —CHI2, —CH₂Cl, —CH₂Br, —CH₂F, —CH₂I, or unsubstituted methyl.

In embodiments, R^{10A} is independently halogen. In embodiments, R^{10A} is independently —CH₂OCH₃. In embodiments, R^{10A} is independently —SO₂CH₃. In embodiments, R^{10A} is independently —SCH₃. In embodiments, R^{10A} is independently —OCH₃. In embodiments, R^{10A} is independently unsubstituted C₁—C₄ alkyl. In embodiments, R^{10A} is independently unsubstituted cyclopropyl. In embodiments, R^{10A} is independently unsubstituted phenyl. In embodiments, R^{10A} is independently hydrogen. In embodiments, R^{10A} is independently —CCl₃. In embodiments, R^{10A} is independently —CBr₃. In embodiments, R^{10A} is independently —CF₃. In embodiments, R^{10A} is independently —CI₃. In embodiments, R^{10A} is independently —CHCl₂. In embodiments, R^{10A} is independently —CHBr₂. In embodiments, R^{10A} is independently —CHF₂. In embodiments, R^{10A} is independently —CHI2. In embodiments, R^{10A} is independently —CH₂Cl. In embodiments, R^{10A} is independently —CH₂Br. In embodiments, R^{10A} is independently —CH₂F. In embodiments, R^{10A} is independently —CH₂I. In embodiments, R^{10A} is independently —CN. In embodiments, R^{10A} is independently —OH. In embodiments, R^{10A} is independently —NH₂. In embodiments, R^{10A} is independently —COOH. In embodiments, R^{10A} is independently —CONH₂. In embodiments, R^{10A} is independently —OCCl₃. In embodiments, R^{10A} is independently —OCF₃. In embodiments, R^{10A} is independently —OCBr₃. In embodiments, R^{10A} is independently —OCI₃. In embodiments, R^{10A} is independently —OCHCl₂. In embodiments, R^{10A} is independently —OCHBr₂. In embodiments, R^{10A} is independently —OCHI2. In embodiments, R^{10A} is independently —OCHF₂. In embodiments, R^{10A} is independently —OCH₂Cl. In embodiments, R^{10A} is independently —OCH₂Br. In embodiments, R^{10A} is independently —OCH₂I. In embodiments, R^{10A} is independently —OCH₂F. In embodiments, R^{10A} is independently halogen. In embodiments, R^{10A} is independently —NO₂. In embodiments, R^{10A} is independently —OCH₃. In embodiments, R^{10A} is independently —OCH₂CH₃. In embodiments, R^{10A} is independently —OCH(CH₃)₂. In embodiments, R^{10A} is independently-OC(CH₃)₃. In embodiments, R^{10A} is independently —CH₃. In embodiments, R^{10A} is independently —CH₂CH₃. In embodiments, R^{10A} is independently —CH(CH₃)₂. In embodiments, R^{10A} is independently —C(CH₃)₃. In embodiments, R^{10A} is independently unsubstituted cyclopropyl. In embodiments, R^{10A} is independently unsubstituted cyclobutyl. In embodiments, R^{10A} is independently unsubstituted cyclopentyl. In embodiments, R^{10A} is independently unsubstituted cyclohexyl. In embodiments, R^{10A} is independently substituted or unsubstituted alkyl (e.g., C₁—C₈, C₁—C₆, or C₁—C₄). In embodiments, R^{10A} is independently substituted or unsubstituted heteroalkyl (e.g., 2 to 8 membered, 2 to 6 membered, or 2 to 4 membered). In embodiments, R^{10A} is independently substituted or unsubstituted cycloalkyl (e.g., C₃—C₈, C₃—C₆, or C₅—C₆). In embodiments, R^{10A} is independently substituted or unsubstituted heterocycloalkyl (e.g., 3 to 8 membered, 3 to 6 membered, or 5 to 6 membered). In embodiments, R^{10A} is independently substituted or unsubstituted aryl (e.g., C₆—C₁₀, C₁₀, or phenyl). In embodiments, R^{10A} is independently substituted or unsubstituted heteroaryl (e.g., 5 to 10 membered, 5 to 9 membered, or 5 to 6 membered). In embodiments, R^{10A} is independently unsubstituted alkyl (e.g., C₁—C₈, C₁—C₆, or C₁—C₄). In embodiments, R^{10A} is independently unsubstituted heteroalkyl (e.g., 2 to 8 membered, 2 to 6 membered, or 2 to 4 membered). In embodiments, R^{10A} is independently unsubstituted cycloalkyl (e.g., C₃-C₈, C₃—C₆, or C₅—C₆). In embodiments, R^{10A} is independently unsubstituted heterocycloalkyl (e.g., 3 to 8 membered, 3 to 6 membered, or 5 to 6 membered). In embodiments, R^{10A} is independently unsubstituted aryl (e.g., C₆—C₁₀, C₁₀, or phenyl). In embodiments, R^{10A} is independently unsubstituted heteroaryl (e.g., 5 to 10 membered, 5 to 9 membered, or 5 to 6 membered).

In embodiments, R^{10B} is independently halogen. In embodiments, R^{10B} is independently —CH₂OCH₃. In embodiments, R^{10B} is independently —SO₂CH₃. In embodiments, R^{10B} is independently —SCH₃. In embodiments, R^{10B} is independently —OCH₃. In embodiments, R^{10B} is independently unsubstituted C₁—C₄ alkyl. In embodiments, R^{10B} is independently unsubstituted cyclopropyl. In embodiments, R^{10B} is independently unsubstituted phenyl. In embodiments, R^{10B} is independently hydrogen. In embodiments, R^{10B} is independently —CCl₃. In embodiments, R^{10B} is independently —CBr₃. In embodiments, R^{10B} is independently —CF₃. In embodiments, R^{10B} is independently —CI₃. In embodiments, R^{10B} is independently —CHCl₂. In embodiments, R^{10B} is independently —CHBr₂. In embodiments, R^{10B} is independently —CHF₂. In embodiments, R^{10B} is independently —CHI2. In embodiments, R^{10B} is independently —CH₂Cl. In embodiments, R^{10B} is independently —CH2Br. In embodiments, R^{10B} is independently —CH₂F. In embodiments, R^{10B} is independently —CH₂I. In embodiments, R^{10B} is independently —CN. In embodiments, R^{10B} is independently —OH. In embodiments, R^{10B} is independently —NH₂. In embodiments, R^{10B} is independently -COOH. In embodiments, R^{10B} is independently —CONH₂. In embodiments, R^{10B} is independently —OCCl₃. In embodiments, R^{10B} is independently —OCF₃. In embodiments, R^{10B} is independently —OCBr₃. In embodiments, R^{10B} is independently —OCI₃. In embodiments, R^{10B} is independently —OCHCl₂. In embodiments, R^{10B} is independently —OCHBr2. In embodiments, R^{10B} is independently —OCHI2. In embodiments, R^{10B} is independently —OCHF₂. In embodiments, R^{10B} is independently —OCH₂Cl. In embodiments, R^{10B} is independently —OCH2Br. In embodiments, R^{10B} is independently —OCH₂I. In embodiments, R^{10B} is independently —OCH₂F. In embodiments, R^{10B} is independently halogen. In embodiments, R^{10B} is independently —NO₂. In embodiments, R^{10B} is independently —OCH₃. In embodiments, R^{10B} is independently -OCH₂CH₃. In embodiments, R^{10B} is independently-OCH(CH₃)₂. In embodiments, R^{10B} is independently-OC(CH₃)₃. In embodiments, R^{10B} is independently —CH₃. In embodiments, R^{10B} is independently —CH₂CH₃. In embodiments, R^{10B} is independently —CH(CH₃)₂. In embodiments, R^{10B} is independently-C(CH₃)₃. In embodiments, R^{10B} is independently unsubstituted cyclopropyl. In embodiments, R^{10B} is independently unsubstituted cyclobutyl. In embodiments, R^{10B} is independently unsubstituted cyclopentyl. In embodiments, R^{10B} is independently unsubstituted cyclohexyl. In embodiments, R^{10B} is independently substituted or unsubstituted alkyl (e.g., C₁—C₈, C₁—C₆, or C₁—C₄). In embodiments, R^{10B} is independently substituted or unsubstituted heteroalkyl (e.g., 2 to 8 membered, 2 to 6 membered, or 2 to 4 membered). In embodiments, R^{10B} is independently substituted or unsubstituted cycloalkyl (e.g., C₃—C₈, C₃—C₆, or C₅—C₆). In embodiments, R^{10B} is independently substituted or unsubstituted heterocycloalkyl (e.g., 3 to 8 membered, 3 to 6 membered, or 5 to 6 membered). In embodiments, R^{10B} is independently substituted or unsubstituted aryl (e.g., C₆—C₁₀, C₁₀, or phenyl). In embodiments, R^{10B} is independently substituted or unsubstituted heteroaryl (e.g., 5 to 10 membered, 5 to 9 membered, or 5 to 6 membered). In embodiments, R^{10B} is independently unsubstituted alkyl (e.g., C₁—C₈, C₁—C₆, or C₁—C₄). In embodiments, R^{10B} is independently unsubstituted heteroalkyl (e.g., 2 to 8 membered, 2 to 6 membered, or 2 to 4 membered). In embodiments, R^{10B} is independently unsubstituted cycloalkyl (e.g., C₃-C₈, C₃—C₆, or C₅—C₆). In embodiments, R^{10B} is independently unsubstituted heterocycloalkyl (e.g., 3 to 8 membered, 3 to 6 membered, or 5 to 6 membered). In embodiments, R^{10B} is independently unsubstituted aryl (e.g., C₆—C₁₀, C₁₀, or phenyl). In embodiments, R^{10B} is independently unsubstituted heteroaryl (e.g., 5 to 10 membered, 5 to 9 membered, or 5 to 6 membered).

In embodiments, R^{10A} and R^{10B} substituents bonded to the same nitrogen atom are joined to form a substituted or unsubstituted heterocycloalkyl (e.g., 3 to 8 membered, 3 to 6 membered, or 5 to 6 membered). In embodiments, R^{10A} and R^{10B} substituents bonded to the same nitrogen atom are joined to form a substituted or unsubstituted heteroaryl (e.g., 5 to 10 membered, 5 to 9 membered, or 5 to 6 membered). In embodiments, R^{10A} and R^{10B} substituents bonded to the same nitrogen atom are joined to form an unsubstituted heterocycloalkyl (e.g., 3 to 8 membered, 3 to 6 membered, or 5 to 6 membered). In embodiments, R^{10A} and R^{10B} substituents bonded to the same nitrogen atom are joined to form an unsubstituted heteroaryl (e.g., 5 to 10 membered, 5 to 9 membered, or 5 to 6 membered).

In embodiments, R^{10C} is independently halogen. In embodiments, R^{10C} is independently —CH₂OCH₃. In embodiments, R^{10C} is independently —SO₂CH₃. In embodiments, R^{10C} is independently —SCH₃. In embodiments, R^{10C} is independently —OCH₃. In embodiments, R^{10C} is independently unsubstituted C₁—C₄ alkyl. In embodiments, R^{10C} is independently unsubstituted cyclopropyl. In embodiments, R^{10C} is independently unsubstituted phenyl. In embodiments, R^{10C} is independently hydrogen. In embodiments, R^{10C} is independently —CCl₃. In embodiments, R^{10C} is independently —CBr₃. In embodiments, R^{10C} is independently —CF₃. In embodiments, R^{10C} is independently —CI₃. In embodiments, R^{10C} is independently —CHCl₂. In embodiments, R^{10C} is independently —CHBr₂. In embodiments, R^{10C} is independently —CHF₂. In embodiments, R^{10C} is independently —CHI2. In embodiments, R^{10C} is independently —CH₂Cl. In embodiments, R^{10C} is independently —CH2Br. In embodiments, R^{10C} is independently —CH₂F. In embodiments, R^{10C} is independently —CH₂I. In embodiments, R^{10C} is independently —CN. In embodiments, R^{10C} is independently —OH. In embodiments, R^{10C} is independently —NH₂. In embodiments, R^{10C} is independently -COOH. In embodiments, R^{10C} is independently —CONH₂. In embodiments, R^{10C} is independently —OCCl₃. In embodiments, R^{10C} is independently —OCF₃. In embodiments, R^{10C} is independently —OCBr₃. In embodiments, R^{10C} is independently —OCI₃. In embodiments, R^{10C} is independently —OCHCl₂. In embodiments, R^{10C} is independently —OCHBr₂. In embodiments, R^{10C} is independently —OCHI2. In embodiments, R^{10C} is independently —OCHF₂. In embodiments, R^{10C} is independently —OCH₂Cl. In embodiments, R^{10C} is independently —OCH2Br. In embodiments, R^{10C} is independently —OCH₂I. In embodiments, R^{10C} is independently —OCH₂F. In embodiments, R^{10C} is independently halogen. In embodiments, R^{10C} is independently —NO₂. In embodiments, R^{10C} is independently —OCH₃. In embodiments, R^{10C} is independently -OCH₂CH₃. In embodiments, R^{10C} is independently —OCH(CH₃)₂. In embodiments, R^{10C} is independently-OC(CH₃)₃. In embodiments, R^{10C} is independently —CH₃. In embodiments, R^{10C} is independently —CH₂CH₃. In embodiments, R^{10C} is independently —CH(CH₃)₂. In embodiments, R^{10C} is independently —C(CH₃)₃. In embodiments, R^{10C} is independently unsubstituted cyclopropyl. In embodiments, R^{10C} is independently unsubstituted cyclobutyl. In embodiments, R^{10C} is independently unsubstituted cyclopentyl. In embodiments, R^{10C} is independently unsubstituted cyclohexyl. In embodiments, R^{10C} is independently substituted or unsubstituted alkyl (e.g., C₁—C₈, C₁—C₆, or C₁—C₄). In embodiments, R^{10C} is independently substituted or unsubstituted heteroalkyl (e.g., 2 to 8 membered, 2 to 6 membered, or 2 to 4 membered). In embodiments, R^{10C} is independently substituted or unsubstituted cycloalkyl (e.g., C₃—C₈, C₃—C₆, or C₅—C₆). In embodiments, R^{10C} is independently substituted or unsubstituted heterocycloalkyl (e.g., 3 to 8 membered, 3 to 6 membered, or 5 to 6 membered). In embodiments, R^{10C} is independently substituted or unsubstituted aryl (e.g., C₆—C₁₀, C₁₀, or phenyl). In embodiments, R^{10C} is independently substituted or unsubstituted heteroaryl (e.g., 5 to 10 membered, 5 to 9 membered, or 5 to 6 membered). In embodiments, R^{10C} is independently unsubstituted alkyl (e.g., C₁—C₈, C₁—C₆, or C₁—C₄). In embodiments, R^{10C} is independently unsubstituted heteroalkyl (e.g., 2 to 8 membered, 2 to 6 membered, or 2 to 4 membered). In embodiments, R^{10C} is independently unsubstituted cycloalkyl (e.g., C₃-C₈, C₃—C₆, or C₅—C₆). In embodiments, R^{10C} is independently unsubstituted heterocycloalkyl (e.g., 3 to 8 membered, 3 to 6 membered, or 5 to 6 membered). In embodiments, R^{10C} is independently unsubstituted aryl (e.g., C₆—C₁₀, C₁₀, or phenyl). In embodiments, R^{10C} is independently unsubstituted heteroaryl (e.g., 5 to 10 membered, 5 to 9 membered, or 5 to 6 membered).

In embodiments, R^{10D} is independently halogen. In embodiments, R^{10D} is independently —CH₂OCH₃. In embodiments, R^{10D} is independently —SO₂CH₃. In embodiments, R^{10D} is independently —SCH₃. In embodiments, R^{10D} is independently —OCH₃. In embodiments, R^{10D} is independently unsubstituted C₁—C₄ alkyl. In embodiments, R^{10D} is independently unsubstituted cyclopropyl. In embodiments, R^{10D} is independently unsubstituted phenyl. In embodiments, R^{10D} is independently hydrogen. In embodiments, R^{10D} is independently —CCl₃. In embodiments, R^{10D} is independently —CBr₃. In embodiments, R^{10D} is independently —CF₃. In embodiments, R^{10D} is independently —CI₃. In embodiments, R^{10D} is independently —CHCl₂. In embodiments, R^{10D} is independently —CHBr₂. In embodiments, R^{10D} is independently —CHF₂. In embodiments, R^{10D} is independently —CHI2. In embodiments, R^{10D} is independently —CH₂Cl. In embodiments, R^{10D} is independently —CH₂Br. In embodiments, R^{10D} is independently —CH₂F. In embodiments, R^{10D} is independently —CH₂I. In embodiments, R^{10D} is independently —CN. In embodiments, R^{10D} is independently —OH. In embodiments, R^{10D} is independently —NH₂. In embodiments, R^{10D} is independently —COOH. In embodiments, R^{10D} is independently —CONH₂. In embodiments, R^{10D} is independently —OCCl₃. In embodiments, R^{10D} is independently —OCF₃. In embodiments, R^{10D} is independently —OCBr₃. In embodiments, R^{10D} is independently —OCI₃. In embodiments, R^{10D} is independently —OCHCl₂. In embodiments, R^{10D} is independently —OCHBr₂. In embodiments, R^{10D} is independently —OCHI2. In embodiments, R^{10D} is independently —OCHF₂. In embodiments, R^{10D} is independently —OCH₂Cl. In embodiments, R^{10D} is independently —OCH₂Br. In embodiments, R^{10D} is independently —OCH₂I. In embodiments, R^{10D} is independently —OCH₂F. In embodiments, R^{10D} is independently halogen. In embodiments, R^{10D} is independently —NO₂. In embodiments, R^{10D} is independently —OCH₃. In embodiments, R^{10D} is independently -OCH₂CH₃. In embodiments, R^{10D} is independently —OCH(CH₃)₂. In embodiments, R^{10D} is independently-OC(CH₃)₃. In embodiments, R^{10D} is independently —CH₃. In embodiments, R^{10D} is independently —CH₂CH₃. In embodiments, R^{10D} is independently —CH(CH₃)₂. In embodiments, R^{10D} is independently —C(CH₃)₃. In embodiments, R^{10D} is independently unsubstituted cyclopropyl. In embodiments, R^{10D} is independently unsubstituted cyclobutyl. In embodiments, R^{10D} is independently unsubstituted cyclopentyl. In embodiments, R^{10D} is independently unsubstituted cyclohexyl. In embodiments, R^{10D} is independently substituted or unsubstituted alkyl (e.g., C₁—C₈, C₁—C₆, or C₁—C₄). In embodiments, R^{10D} is independently substituted or unsubstituted heteroalkyl (e.g., 2 to 8 membered, 2 to 6 membered, or 2 to 4 membered). In embodiments, R^{10D} is independently substituted or unsubstituted cycloalkyl (e.g., C₃—C₈, C₃—C₆, or C₅—C₆). In embodiments, R^{10D} is independently substituted or unsubstituted heterocycloalkyl (e.g., 3 to 8 membered, 3 to 6 membered, or 5 to 6 membered). In embodiments, R^{10D} is independently substituted or unsubstituted aryl (e.g., C₆—C₁₀, C₁₀, or phenyl). In embodiments, R^{10D} is independently substituted or unsubstituted heteroaryl (e.g., 5 to 10 membered, 5 to 9 membered, or 5 to 6 membered). In embodiments, R^{10D} is independently unsubstituted alkyl (e.g., C₁—C₈, C₁—C₆, or C₁—C₄). In embodiments, R^{10D} is independently unsubstituted heteroalkyl (e.g., 2 to 8 membered, 2 to 6 membered, or 2 to 4 membered). In embodiments, R^{10D} is independently unsubstituted cycloalkyl (e.g., C₃-C₈, C₃—C₆, or C₅—C₆). In embodiments, R^{10D} is independently unsubstituted heterocycloalkyl (e.g., 3 to 8 membered, 3 to 6 membered, or 5 to 6 membered). In embodiments, R^{10D} is independently unsubstituted aryl (e.g., C₆—C₁₀, C₁₀, or phenyl). In embodiments, R^{10D} is independently unsubstituted heteroaryl (e.g., 5 to 10 membered, 5 to 9 membered, or 5 to 6 membered). R^{10D} is independently hydrogen or unsubstituted C₁—C₄ alkyl.

In embodiments, R^{10.A}, R¹⁰^{.}^{B}, R^{10.C}, R^{10.D}, and R¹⁰^{.}^{E} are independently halogen, —OH, —CF₃, —CHF₂, —CH₂F, —OCF₃, —OCH₂F, —OCHF₂, —OCH₃, —SCH₃, —OCH₃, unsubstituted C₁—C₄ alkyl, unsubstituted cyclopropyl, unsubstituted morpholinyl, or unsubstituted piperazinyl, or unsubstituted phenyl. In embodiments, R^{10.A}, R^{10.B}, R^{10.C}, R^{10.D}, and R^{10.E} are independently —F, —Cl, —CH₃, —OCH₃, —OH, unsubstituted morpholinyl, or unsubstituted piperazinyl.

In embodiments, R^{10.C} is independently unsubstituted C₁—C₄ alkyl. In embodiments, R^{10.C} is independently hydrogen.

In embodiments, R^{10.A} is independently hydrogen, halogen, —CX^{10.A}₃, —CHX^{10.A}₂, —CH₂X¹⁰^{.A}, —OCX^{10.A}₃, —OCH₂X¹⁰^{.A}, —OCHX^{10.A}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D},—NR^{10A}C(O)R^{10C}, —NR^{10A}C(O)OR^{10C}, —NR^{10A}OR^{10C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered). X^{10.A} is independently halogen.

In embodiments, R^{10.A} is independently hydrogen, halogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R^{10.A} is independently hydrogen. In embodiments, R^{10.A} is independently halogen. In embodiments, R^{10.A} is independently —CX^{10.A}₃. In embodiments, R^{10.A} is independently —CHX^{10.A}₂. In embodiments, R^{10.A} is independently —CH₂X^{10.A}. In embodiments, R^{10.A} is independently —OCX^{10.A}₃. In embodiments, R^{10.A} is independently —OCH₂X^{10.A}. In embodiments, R^{10.A} is independently —OCHX^{10.A}₂. In embodiments, R^{10.A} is independently —CN. In embodiments, R^{10.A} is independently —SOₙ₁₀R^{10D}. In embodiments, R^{10.A} is independently —SOᵥ₁₀NR^{10A}R^{10B}. In embodiments, R^{10.A} is independently —NR^{10C}NR^{10A}R^{10B}. In embodiments, R^{10.A} is independently —ONR^{10A}R^{10B}. In embodiments, R^{10.A} is independently —NHC(O)NR^{10C}NR^{10A}R^{10B}. In embodiments, R^{10.A} is independently —NHC(O)NR^{10A}R^{10B}. In embodiments, R^{10.A} is independently -N(O)ₘ₁₀. In embodiments, R^{10.A} is independently -NR^{10A}R^{10B}. In embodiments, R^{10.A} is independently —C(O)R^{10C}. In embodiments, R^{10.A} is independently —C(O)—OR^{10C}. In embodiments, R^{10.A} is independently —C(O)NR^{10A}R^{10B}. In embodiments, R^{10.A} is independently -OR^{10D}. In embodiments, R^{10.A} is independently —NR^{10A}SO₂R^{10D}. In embodiments, R^{10.A} is independently —NR^{10A}C(O)R^{10C}. In embodiments, R^{10.A} is independently —NR^{10A}C(O)OR^{10C}. In embodiments, R^{10.A} is independently —NR^{10A}OR^{10C}. In embodiments, R^{10.A} is independently —SF₅. In embodiments, R^{10.A} is independently —N₃. In embodiments, R^{10.A} is independently —F. In embodiments, R^{10.A} is independently —Cl. In embodiments, R^{10.A} is independently —Br. In embodiments, R^{10.A} is independently —I. In embodiments, R^{10.A} is independently —CH₂OCH₃. In embodiments, R^{10.A} is independently —SO₂CH₃. In embodiments, R^{10.A} is independently —SCH₃. In embodiments, R^{10.A} is independently —OCH₃. In embodiments, R^{10.A} is independently —CH₂CH₂OCH₃. In embodiments, R^{10.A} is independently —SO₂CH₂CH₃. In embodiments, R^{10.A} is independently —SCH2CH3. In embodiments, R^{10.A} is independently —OCH2CH3. In embodiments, R^{10.A} is independently —CH₂OCH₂CH₃. In embodiments, R^{10.A} is independently unsubstituted C₁—C₄ alkyl. In embodiments, R^{10.A} is independently unsubstituted cyclopropyl. In embodiments, R^{10.A} is independently unsubstituted phenyl. In embodiments, R^{10.A} is independently hydrogen. In embodiments, R^{10.A} is independently —CCl₃. In embodiments, R^{10.A} is independently —CBr₃. In embodiments, R^{10.A} is independently —CF₃. In embodiments, R^{10.A} is independently —CI₃. In embodiments, R^{10.A} is independently —CHCl₂. In embodiments, R^{10.A} is independently —CHBr₂. In embodiments, R^{10.A} is independently —CHF₂. In embodiments, R^{10.A} is independently —CHI2. In embodiments, R^{10.A} is independently —CH₂Cl. In embodiments, R^{10.A} is independently -CH2Br. In embodiments, R^{10.A} is independently —CH₂F. In embodiments, R^{10.A} is independently —CH₂I. In embodiments, R^{10.A} is independently —CN. In embodiments, R^{10.A} is independently —OH. In embodiments, R^{10.A} is independently —NH₂. In embodiments, R^{10.A} is independently -COOH. In embodiments, R^{10.A} is independently —CONH₂. In embodiments, R^{10.A} is independently —OCCl₃. In embodiments, R^{10.A} is independently —OCF₃. In embodiments, R^{10.A} is independently —OCBr₃. In embodiments, R^{10.A} is independently —OCI₃. In embodiments, R^{10.A} is independently —OCHCl₂. In embodiments, R^{10.A} is independently -OCHBr₂. In embodiments, R^{10.A} is independently —OCHI2. In embodiments, R^{10.A} is independently —OCHF₂. In embodiments, R^{10.A} is independently —OCH₂Cl. In embodiments, R^{10.A} is independently —OCH₂Br. In embodiments, R^{10.A} is independently —OCH₂I. In embodiments, R^{10.A} is independently —OCH₂F. In embodiments, R^{10.A} is independently halogen. In embodiments, R^{10.A} is independently —NO₂. In embodiments, R^{10.A} is independently —OCH₃. In embodiments, R^{10.A} is independently —OCH₂CH₃. In embodiments, R^{10.A} is independently —OCH(CH₃)₂. In embodiments, R^{10.A} is independently —OC(CH₃)₃. In embodiments, R^{10.A} is independently —CH₃. In embodiments, R^{10.A} is independently -CH₂CH₃. In embodiments, R^{10.A} is independently —CH(CH₃)₂. In embodiments, R^{10.A} is independently —C(CH₃)₃. In embodiments, R^{10.A} is independently unsubstituted cyclopropyl. In embodiments, R^{10.A} is independently unsubstituted cyclobutyl. In embodiments, R^{10.A} is independently unsubstituted cyclopentyl. In embodiments, R^{10.A} is independently unsubstituted cyclohexyl. In embodiments, R^{10.A} is independently substituted or unsubstituted alkyl (e.g., C₁—C₈, C₁—C₆, or C₁—C₄). In embodiments, R^{10.A} is independently substituted or unsubstituted heteroalkyl (e.g., 2 to 8 membered, 2 to 6 membered, or 2 to 4 membered). In embodiments, R^{10.A} is independently substituted or unsubstituted cycloalkyl (e.g., C₃—C₈, C₃—C₆, or C₅—C₆). In embodiments, R^{10.A} is independently substituted or unsubstituted heterocycloalkyl (e.g., 3 to 8 membered, 3 to 6 membered, or 5 to 6 membered). In embodiments, R^{10.A} is independently substituted or unsubstituted aryl (e.g., C₆—C₁₀, C₁₀, or phenyl). In embodiments, R^{10.A} is independently substituted or unsubstituted heteroaryl (e.g., 5 to 10 membered, 5 to 9 membered, or 5 to 6 membered). In embodiments, R^{10.A} is independently unsubstituted alkyl (e.g., C₁—C₈, C₁—C₆, or C₁—C₄). In embodiments, R^{10.A} is independently unsubstituted heteroalkyl (e.g., 2 to 8 membered, 2 to 6 membered, or 2 to 4 membered). In embodiments, R^{10.A} is independently unsubstituted cycloalkyl (e.g., C₃-C₈, C₃—C₆, or C₅—C₆). In embodiments, R^{10.A} is independently unsubstituted heterocycloalkyl (e.g., 3 to 8 membered, 3 to 6 membered, or 5 to 6 membered). In embodiments, R^{10.A} is independently unsubstituted aryl (e.g., C₆—C₁₀, C₁₀, or phenyl). In embodiments, R^{10.A} is independently unsubstituted heteroaryl (e.g., 5 to 10 membered, 5 to 9 membered, or 5 to 6 membered). In embodiments, R^{10.A} is independently —F. In embodiments, R^{10.A} is independently —Cl. In embodiments, R^{10.A} is independently —CH₃. In embodiments, R^{10.A} is independently —OCH₃. In embodiments, R^{10.A} is independently —OH. In embodiments, R^{10.A} is independently unsubstituted morpholinyl. In embodiments, R^{10.A} is independently unsubstituted piperazinyl. In embodiments, X^{10.A} is independently —F. In embodiments, X^{10.A} is independently —Cl. In embodiments, X^{10.A} is independently —Br. In embodiments, X^{10.A} is independently —I.

In embodiments, R^{10.B} is independently hydrogen, halogen, —CX^{10.B}₃, —CHX^{10.B}₂, —CH₂X^{10.B}, —OCX^{10.B}₃, —OCH₂X^{10.B}, —OCHX^{10.B}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered),. X^{10.B} is independently halogen.

In embodiments, R^{10.B} is independently hydrogen, halogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R^{10.B} is independently hydrogen. In embodiments, R^{10.B} is independently halogen. In embodiments, R^{10.B} is independently -CX^{10.B}₃. In embodiments, R^{10.B} is independently -CHX^{10.B}₂. In embodiments, R^{10.B} is independently —CH₂X^{10.B}. In embodiments, R^{10.B} is independently -OCX^{10.B}₃. In embodiments, R^{10.B} is independently —OCH₂X^{10.B}. In embodiments, R^{10.B} is independently —OCHX^{10.B}₂. In embodiments, R^{10.B} is independently —CN. In embodiments, R^{10.B} is independently -SOₙ₁₀R^{10D}. In embodiments, R^{10.B} is independently -SOᵥioNR^{10A}R^{10B}. In embodiments, R^{10.B} is independently -NR^{10C}NR^{10A}R^{10B}. In embodiments, R^{10.B} is independently -ONR^{10A}R^{10B}. In embodiments, R^{10.B} is independently —NHC(O)NR^{10C}NR^{10A}R^{10B}. In embodiments, R^{10.B} is independently —NHC(O)NR^{10A}R^{10B}. In embodiments, R^{10.B} is independently —N(O)ₘ₁₀. In embodiments, R^{10.B} is independently —NR^{10A}R^{10B}. In embodiments, R^{10.B} is independently —C(O)R^{10C}. In embodiments, R^{10.B} is independently —C(O)—OR^{10C}. In embodiments, R^{10.B} is independently —C(O)NR^{10A}R^{10B}. In embodiments, R^{10.B} is independently —OR^{10D}. In embodiments, R^{10.B} is independently -NR^{10A}SO₂R^{10D}. In embodiments, R^{10.B} is independently —NR¹⁰AC(O)R^{10C}. In embodiments, R^{10.B} is independently —NR^{10A}C(O)OR^{10C}. In embodiments, R^{10.B} is independently —NR^{10A}OR^{10C}. In embodiments, R^{10.B} is independently —SF₅. In embodiments, R^{10.B} is independently —N₃. In embodiments, R^{10.B} is independently —F. In embodiments, R^{10.B} is independently —Cl. In embodiments, R^{10.B} is independently —Br. In embodiments, R^{10.B} is independently —I. In embodiments, R^{10.B} is independently —CH₂OCH₃. In embodiments, R^{10.B} is independently —SO₂CH₃. In embodiments, R^{10.B} is independently —SCH₃. In embodiments, R^{10.B} is independently —OCH₃. In embodiments, R^{10.B} is independently —CH₂CH₂OCH₃. In embodiments, R^{10.B} is independently —SO₂CH₂CH₃. In embodiments, R^{10.B} is independently —SCH2CH3. In embodiments, R^{10.B} is independently —OCH2CH3. In embodiments, R^{10.B} is independently —CH₂OCH₂CH₃. In embodiments, R^{10.B} is independently unsubstituted C₁—C₄ alkyl. In embodiments, R^{10.B} is independently unsubstituted cyclopropyl. In embodiments, R^{10.B} is independently unsubstituted phenyl. In embodiments, R^{10.B} is independently hydrogen. In embodiments, R^{10.B} is independently —CCl₃. In embodiments, R^{10.B} is independently —CBr₃. In embodiments, R^{10.B} is independently —CF₃. In embodiments, R^{10.B} is independently —CI₃. In embodiments, R^{10.B} is independently —CHC1₂. In embodiments, R^{10.B} is independently —CHBr₂. In embodiments, R^{10.B} is independently —CHF₂. In embodiments, R^{10.B} is independently —CHI2. In embodiments, R^{10.B} is independently —CH₂C1. In embodiments, R^{10.B} is independently —CH₂Br. In embodiments, R^{10.B} is independently —CH₂F. In embodiments, R^{10.B} is independently —CH₂I. In embodiments, R^{10.B} is independently —CN. In embodiments, R^{10.B} is independently —OH. In embodiments, R^{10.B} is independently —NH₂. In embodiments, R^{10.B} is independently —COOH. In embodiments, R^{10.B} is independently —CONH₂. In embodiments, R^{10.B} is independently —OCC1₃. In embodiments, R^{10.B} is independently —OCF₃. In embodiments, R^{10.B} is independently —OCBr₃. In embodiments, R^{10.B} is independently —OCI₃. In embodiments, R^{10.B} is independently —OCHC1₂. In embodiments, R^{10.B} is independently —OCHBr₂. In embodiments, R^{10.B} is independently —OCHI2. In embodiments, R^{10.B} is independently —OCHF₂. In embodiments, R^{10.B} is independently —OCH₂C1. In embodiments, R^{10.B} is independently —OCH₂Br. In embodiments, R^{10.B} is independently -OCH₂I. In embodiments, R^{10.B} is independently —OCH₂F. In embodiments, R^{10.B} is independently halogen. In embodiments, R^{10.B} is independently —NO₂. In embodiments, R^{10.B} is independently —OCH₃. In embodiments, R^{10.B} is independently-OCH₂CH₃. In embodiments, R^{10.B} is independently —OCH(CH₃)₂. In embodiments, R^{10.B} is independently -OC(CH₃)₃. In embodiments, R^{10.B} is independently —CH₃. In embodiments, R^{10.B} is independently —CH₂CH₃. In embodiments, R^{10.B} is independently —CH(CH₃)₂. In embodiments, R^{10.B} is independently —C(CH₃)₃. In embodiments, R^{10.B} is independently unsubstituted cyclopropyl. In embodiments, R^{10.B} is independently unsubstituted cyclobutyl. In embodiments, R^{10.B} is independently unsubstituted cyclopentyl. In embodiments, R^{10.B} is independently unsubstituted cyclohexyl. In embodiments, R^{10.B} is independently substituted or unsubstituted alkyl (e.g., C₁—C₈, C₁—C₆, or C₁—C₄). In embodiments, R^{10.B} is independently substituted or unsubstituted heteroalkyl (e.g., 2 to 8 membered, 2 to 6 membered, or 2 to 4 membered). In embodiments, R^{10.B} is independently substituted or unsubstituted cycloalkyl (e.g., C₃—C₈, C₃—C₆, or C₅—C₆). In embodiments, R^{10.B} is independently substituted or unsubstituted heterocycloalkyl (e.g., 3 to 8 membered, 3 to 6 membered, or 5 to 6 membered). In embodiments, R^{10.B} is independently substituted or unsubstituted aryl (e.g., C₆—C₁₀, C₁₀, or phenyl). In embodiments, R^{10.B} is independently substituted or unsubstituted heteroaryl (e.g., 5 to 10 membered, 5 to 9 membered, or 5 to 6 membered). In embodiments, R^{10.B} is independently unsubstituted alkyl (e.g., C₁—C₈, C₁—C₆, or C₁—C₄). In embodiments, R^{10.B} is independently unsubstituted heteroalkyl (e.g., 2 to 8 membered, 2 to 6 membered, or 2 to 4 membered). In embodiments, R^{10.B} is independently unsubstituted cycloalkyl (e.g., C₃-C₈, C₃—C₆, or C₅—C₆). In embodiments, R^{10.B} is independently unsubstituted heterocycloalkyl (e.g., 3 to 8 membered, 3 to 6 membered, or 5 to 6 membered). In embodiments, R^{10.B} is independently unsubstituted aryl (e.g., C₆—C₁₀, C₁₀, or phenyl). In embodiments, R^{10.B} is independently unsubstituted heteroaryl (e.g., 5 to 10 membered, 5 to 9 membered, or 5 to 6 membered). In embodiments, R^{10.B} is independently —F. In embodiments, R^{10.B} is independently —Cl. In embodiments, R^{10.B} is independently —CH₃. In embodiments, R^{10.B} is independently —OCH₃. In embodiments, R^{10.B} is independently —OH. In embodiments, R^{10.B} is independently unsubstituted morpholinyl. In embodiments, R^{10.B} is independently unsubstituted piperazinyl. In embodiments, X^{10 B} is independently —F. In embodiments, X^{10 B} is independently —Cl. In embodiments, X^{10 B} is independently —Br. In embodiments, X^{10 B} is independently —I.

In embodiments, R^{10.C} is independently hydrogen, halogen, -CX^{10.C}3, -CHX^{10.C}₂, —CH₂X^{10.C}, —OCX^{10.C}₃, —OCH₂X^{10.C}, —OCHX^{10.C}₂ ,—CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR¹⁰AC(O)R^{10C}, —NR^{10A}C(O)OR^{10C}, —NR^{10A}OR^{10C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆-C₁₀, Cₗo, or phenyl), or substituted or unsubstituted heteroaryl (e.g., 5 to 10 membered, 5 to 9 membered, or 5 to 6 membered),. X^{10.C} is independently halogen.

In embodiments, R^{10.C} is independently hydrogen, halogen, —CCl₃, —CBr₃, —CF₃, —CI₃, —CHCl₂, —CHBr₂, —CHF₂, —CHI2, —CH₂C1, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCC1₃, —OCF₃, —OCBr₃, —OCI₃, —OCHC1₂, —OCHBr₂, —OCHI₂, —OCHF₂, —OCH₂C1, —OCH₂Br, —OCH₂I, —OCH₂F, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R^{10.C} is independently hydrogen. In embodiments, R^{10.C} is independently halogen. In embodiments, R^{10.C} is independently —CX^{10.C}₃. In embodiments, R^{10.C} is independently —CHX^{10.C}₂. In embodiments, R^{10.C} is independently —CH₂X^{10.C}. In embodiments, R^{10.C} is independently -OCX^{10.C}₃. In embodiments, R^{10.C} is independently —OCH₂X^{10.C}. In embodiments, R^{10.C} is independently —OCHX^{10.C}₂. In embodiments, R^{10.C} is independently —CN. In embodiments, R^{10.C} is independently —SOₙ₁₀R^{10D}. In embodiments, R^{10.C} is independently —SOᵥ₁₀NR^{10A}R^{10B}. In embodiments, R^{10.C} is independently —NR^{10C}NR^{10A}R^{10B}. In embodiments, R^{10.C} is independently —ONR^{10A}R^{10B}. In embodiments, R^{10.C} is independently —NHC(O)NR^{10C}NR^{10A}R^{10B}. In embodiments, R^{10.C} is independently —NHC(O)NR^{10A}R^{10B}. In embodiments, R^{10.C} is independently —N(O)ₘ₁₀. In embodiments, R^{10.C} is independently -NR^{10A}R^{10B}. In embodiments, R^{10.C} is independently —C(O)R^{10C}. In embodiments, R^{10.C} is independently —C(O)—OR^{10C}. In embodiments, R^{10.C} is independently —C(O)NR^{10A}R^{10B}. In embodiments, R^{10.C} is independently —OR^{10D}. In embodiments, R^{10.C} is independently -NR^{10A}SO₂R^{10D}. In embodiments, R^{10.C} is independently —NR¹⁰AC(O)R^{10C}. In embodiments, R^{10.C} is independently —NR^{10A}C(O)OR^{10C}. In embodiments, R^{10.C} is independently —NR^{10A}OR^{10C}. In embodiments, R^{10.C} is independently —SF₅. In embodiments, R^{10.C} is independently —N₃. In embodiments, R^{10.C} is independently —F. In embodiments, R^{10.C} is independently —Cl. In embodiments, R^{10.C} is independently —Br. In embodiments, R^{10.C} is independently —I. In embodiments, R^{10.C} is independently —CH_{2O}CH₃. In embodiments, R^{10.C} is independently —SO₂CH₃. In embodiments, R^{10.C} is independently —SCH₃. In embodiments, R^{10.C} is independently —OCH₃. In embodiments, R^{10.C} is independently —CH₂CH₂OCH₃. In embodiments, R^{10.C} is independently —SO₂CH₂CH₃. In embodiments, R^{10.C} is independently —SCH2CH3. In embodiments, R^{10.C} is independently —OCH2CH3. In embodiments, R^{10.C} is independently —CH₂OCH₂CH₃. In embodiments, R^{10.C} is independently unsubstituted C₁—C₄ alkyl. In embodiments, R^{10.C} is independently unsubstituted cyclopropyl. In embodiments, R^{10.C} is independently unsubstituted phenyl. In embodiments, R^{10.C} is independently hydrogen. In embodiments, R^{10.C} is independently —CCl₃. In embodiments, R^{10.C} is independently —CBr₃. In embodiments, R^{10.C} is independently —CF₃. In embodiments, R^{10.C} is independently —CI₃. In embodiments, R^{10.C} is independently —CHC1₂. In embodiments, R^{10.C} is independently —CHBr₂. In embodiments, R^{10.C} is independently —CHF₂. In embodiments, R^{10.C} is independently —CHI2. In embodiments, R^{10.C} is independently —CH₂C1. In embodiments, R^{10.C} is independently —CH₂Br. In embodiments, R^{10.C} is independently —CH₂F. In embodiments, R^{10.C} is independently —CH₂I. In embodiments, R^{10.C} is independently —CN. In embodiments, R^{10.C} is independently —OH. In embodiments, R^{10.C} is independently —NH₂. In embodiments, R^{10.C} is independently —COOH. In embodiments, R^{10.C} is independently —CONH₂. In embodiments, R^{10.C} is independently —OCC1₃. In embodiments, R^{10.C} is independently —OCF₃. In embodiments, R^{10.C} is independently —OCBr₃. In embodiments, R^{10.C} is independently —OCI₃. In embodiments, R^{10.C} is independently —OCHC1₂. In embodiments, R^{10.C} is independently —OCHBr₂. In embodiments, R^{10.C} is independently —OCHI2. In embodiments, R^{10.C} is independently —OCHF₂. In embodiments, R^{10.C} is independently —OCH₂C1. In embodiments, R^{10.C} is independently —OCH₂Br. In embodiments, R^{10.C} is independently —OCH₂I. In embodiments, R^{10.C} is independently —OCH₂F. In embodiments, R^{10.C} is independently halogen. In embodiments, R^{10.C} is independently —NO₂. In embodiments, R^{10.C} is independently —OCH₃. In embodiments, R^{10.C} is independently —OCH₂CH₃. In embodiments, R^{10.C} is independently —OCH(CH₃)2. In embodiments, R^{10.C} is independently —OC(CH₃)3. In embodiments, R^{10.C} is independently —CH₃. In embodiments, R^{10.C} is independently —CH₂CH₃. In embodiments, R^{10.C} is independently —CH(CH₃)₂. In embodiments, R^{10.C} is independently —C(CH₃)₃. In embodiments, R^{10.C} is independently unsubstituted cyclopropyl. In embodiments, R^{10.C} is independently unsubstituted cyclobutyl. In embodiments, R^{10.C} is independently unsubstituted cyclopentyl. In embodiments, R^{10.C} is independently unsubstituted cyclohexyl. In embodiments, R^{10.C} is independently substituted or unsubstituted alkyl (e.g., C₁—C₈, C₁—C₆, or C₁—C₄). In embodiments, R^{10.C} is independently substituted or unsubstituted heteroalkyl (e.g., 2 to 8 membered, 2 to 6 membered, or 2 to 4 membered). In embodiments, R^{10.C} is independently substituted or unsubstituted cycloalkyl (e.g., C₃—C₈, C₃—C₆, or C₅—C₆). In embodiments, R^{10.C} is independently substituted or unsubstituted heterocycloalkyl (e.g., 3 to 8 membered, 3 to 6 membered, or 5 to 6 membered). In embodiments, R^{10.C} is independently substituted or unsubstituted aryl (e.g., C₆—C₁₀, C₁₀, or phenyl). In embodiments, R^{10.C} is independently substituted or unsubstituted heteroaryl (e.g., 5 to 10 membered, 5 to 9 membered, or 5 to 6 membered). In embodiments, R^{10.C} is independently unsubstituted alkyl (e.g., C₁—C₈, C₁—C₆, or C₁—C₄). In embodiments, R^{10.C} is independently unsubstituted heteroalkyl (e.g., 2 to 8 membered, 2 to 6 membered, or 2 to 4 membered). In embodiments, R^{10.C} is independently unsubstituted cycloalkyl (e.g., C₃-C₈, C₃—C₆, or C₅—C₆). In embodiments, R^{10.C} is independently unsubstituted heterocycloalkyl (e.g., 3 to 8 membered, 3 to 6 membered, or 5 to 6 membered). In embodiments, R^{10.C} is independently unsubstituted aryl (e.g., C₆—C₁₀, C₁₀, or phenyl). In embodiments, R^{10.C} is independently unsubstituted heteroaryl (e.g., 5 to 10 membered, 5 to 9 membered, or 5 to 6 membered). In embodiments, R^{10.C} is a substituted or unsubstituted cycloalkyl or substituted or unsubstituted heterocycloalkyl. In embodiments, R^{10.C} is a substituted or unsubstituted C₆ cycloalkyl or substituted or unsubstituted 6 membered heterocycloalkyl. In embodiments, R^{10.C} is a substituted or unsubstituted 6 membered heterocycloalkyl. In embodiments, R^{10.C} is a substituted or unsubstituted piperazinyl. In embodiments, R^{10.C} is independently —F. In embodiments, R^{10.C} is independently —Cl. In embodiments, R^{10.C} is independently —CH₃. In embodiments, R^{10.C} is independently —OCH₃. In embodiments, R^{10.C} is independently —OH. In embodiments, R^{10.C} is independently unsubstituted morpholinyl. In embodiments, R^{10.C} is independently unsubstituted piperazinyl. In embodiments, X^{10.C} is independently —F. In embodiments, X^{10.C} is independently —Cl. In embodiments, X^{10.C} is independently —Br. In embodiments, X^{10.C} is independently —I.

In embodiments, R^{10.D} is independently hydrogen, halogen, —CX^{10.D}₃, —CHX^{10.D}₂, —CH₂X^{10.D}, —OCX^{10.D}3, —OCH₂X^{10.D}, —OCHX^{10.D}₂, —CN, —SOₙ₁₀R^{10D}, —SOᵥ₁₀NR^{10A}R^{10B}, —NR¹⁰CNR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR¹⁰AC(O)OR^{10C}, —NR^{10A}OR^{10C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered),. X^{10.D} is independently halogen.

In embodiments, R^{10.D} is independently hydrogen, halogen, —CCl₃, —CBr₃, —CF₃, —CI₃, —CHC1₂, —CHBr₂, —CHF₂, —CHI2, —CH₂C1, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCC1₃, —OCF₃, —OCBr₃, —OCI₃, —OCHC1₂, —OCHBr₂, —OCHI₂, —OCHF₂, —OCH₂C1, —OCH₂Br, —OCH₂I, —OCH₂F, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R^{10.D} is independently hydrogen. In embodiments, R^{10.D} is independently halogen. In embodiments, R^{10.D} is independently -CX^{10.D}₃. In embodiments, R^{10.D} is independently -CHX^{10.D}₂. In embodiments, R^{10.D} is independently —CH₂X^{10.D}. In embodiments, R^{10.D} is independently —OCX^{10.D}₃. In embodiments, R^{10.D} is independently —OCH₂X^{10.D}. In embodiments, R^{10.D} is independently —OCHX^{10.D}₂. In embodiments, R^{10.D} is independently —CN. In embodiments, R^{10.D} is independently —SOₙ₁₀R^{10D}. In embodiments, R^{10.D} is independently —SOᵥ₁₀NR^{10A}R^{10B}. In embodiments, R^{10.D} is independently —NR^{10C}NR^{10A}R^{10B}. In embodiments, R^{10.D} is independently —ONR^{10A}R^{10B}. In embodiments, R^{10.D} is independently —NHC(O)NR^{10C}NR^{10A}R^{10B}. In embodiments, R^{10.D} is independently —NHC(O)NR^{10A}R^{10B}. In embodiments, R^{10.D} is independently —N(O)ₘ₁₀. In embodiments, R^{10.D} is independently —NR^{10A}R^{10B}. In embodiments, R^{10.D} is independently —C(O)R^{10C}. In embodiments, R^{10.D} is independently —C(O)—OR^{10C}. In embodiments, R^{10.D} is independently —C(O)NR^{10A}R^{10B}. In embodiments, R^{10.D} is independently —OR^{10D}. In embodiments, R^{10.D} is independently -NR^{10A}SO₂R^{10D}. In embodiments, R^{10.D} is independently —NR^{10A}C(O)R^{10C}. In embodiments, R^{10.D} is independently —NR^{10A}C(O)OR^{10C}. In embodiments, R^{10.D} is independently —NR^{10A}OR^{10C}. In embodiments, R^{10.D} is independently —SF₅. In embodiments, R^{10.D} is independently —N₃. In embodiments, R^{10.D} is independently —F. In embodiments, R^{10.D} is independently —Cl. In embodiments, R^{10.D} is independently —Br. In embodiments, R^{10.D} is independently —I. In embodiments, R^{10.D} is independently —CH₂OCH₃. In embodiments, R^{10.D} is independently —SO₂CH₃. In embodiments, R^{10.D} is independently —SCH₃. In embodiments, R^{10.D} is independently —OCH₃. In embodiments, R^{10.D} is independently —CH₂CH₂OCH₃. In embodiments, R^{10.D} is independently —SO₂CH₂CH₃. In embodiments, R^{10.D} is independently —SCH2CH3. In embodiments, R^{10.D} is independently —OCH2CH3. In embodiments, R^{10.D} is independently —CH₂OCH₂CH₃. In embodiments, R^{10.D} is independently unsubstituted C₁—C₄ alkyl. In embodiments, R^{10.D} is independently unsubstituted cyclopropyl. In embodiments, R^{10.D} is independently unsubstituted phenyl. In embodiments, R^{10.D} is independently hydrogen. In embodiments, R^{10.D} is independently -CCl₃. In embodiments, R^{10.D} is independently —CBr₃. In embodiments, R^{10.D} is independently —CF₃. In embodiments, R^{10.D} is independently —CI₃. In embodiments, R^{10.D} is independently —CHCl₂. In embodiments, R^{10.D} is independently —CHBr₂. In embodiments, R^{10.D} is independently —CHF₂. In embodiments, R^{10.D} is independently —CHI₂. In embodiments, R^{10.D} is independently —CH₂Cl. In embodiments, R^{10.D} is independently -CH2Br. In embodiments, R^{10.D} is independently —CH₂F. In embodiments, R^{10.D} is independently —CH₂I. In embodiments, R^{10.D} is independently —CN. In embodiments, R^{10.D} is independently —OH. In embodiments, R^{10.D} is independently —NH₂. In embodiments, R^{10.D} is independently —COOH. In embodiments, R^{10.D} is independently —CONH₂. In embodiments, R^{10.D} is independently —OCCl₃. In embodiments, R^{10.D} is independently —OCF₃. In embodiments, R^{10.D} is independently —OCBr₃. In embodiments, R^{10.D} is independently —OCI₃. In embodiments, R^{10.D} is independently —OCHCl₂. In embodiments, R^{10.D} is independently —OCHBr₂. In embodiments, R^{10.D} is independently —OCHI₂. In embodiments, R^{10.D} is independently —OCHF₂. In embodiments, R^{10.D} is independently —OCH₂Cl. In embodiments, R^{10.D} is independently —OCH₂Br. In embodiments, R^{10.D} is independently —OCH₂I. In embodiments, R^{10.D} is independently —OCH₂F. In embodiments, R^{10.D} is independently halogen. In embodiments, R^{10.D} is independently —NO₂. In embodiments, R^{10.D} is independently —OCH₃. In embodiments, R^{10.D} is independently —OCH₂CH₃. In embodiments, R^{10.D} is independently —OCH(CH₃)2. In embodiments, R^{10.D} is independently —OC(CH₃)3. In embodiments, R^{10.D} is independently —CH₃. In embodiments, R^{10.D} is independently —CH₂CH₃. In embodiments, R^{10.D} is independently —CH(CH₃)₂. In embodiments, R^{10.D} is independently —C(CH₃)₃. In embodiments, R^{10.D} is independently unsubstituted cyclopropyl. In embodiments, R^{10.D} is independently unsubstituted cyclobutyl. In embodiments, R^{10.D} is independently unsubstituted cyclopentyl. In embodiments, R^{10.D} is independently unsubstituted cyclohexyl. In embodiments, R^{10.D} is independently substituted or unsubstituted alkyl (e.g., C₁—C₈, C₁—C₆, or C₁—C₄). In embodiments, R^{10.D} is independently substituted or unsubstituted heteroalkyl (e.g., 2 to 8 membered, 2 to 6 membered, or 2 to 4 membered). In embodiments, R^{10.D} is independently substituted or unsubstituted cycloalkyl (e.g., C₃—C₈, C₃—C₆, or C₅—C₆). In embodiments, R^{10.D} is independently substituted or unsubstituted heterocycloalkyl (e.g., 3 to 8 membered, 3 to 6 membered, or 5 to 6 membered). In embodiments, R^{10.D} is independently substituted or unsubstituted aryl (e.g., C₆—C₁₀, C₁₀, or phenyl). In embodiments, R^{10.D} is independently substituted or unsubstituted heteroaryl (e.g., 5 to 10 membered, 5 to 9 membered, or 5 to 6 membered). In embodiments, R^{10.D} is independently unsubstituted alkyl (e.g., C₁—C₈, C₁—C₆, or C₁—C₄). In embodiments, R^{10.D} is independently unsubstituted heteroalkyl (e.g., 2 to 8 membered, 2 to 6 membered, or 2 to 4 membered). In embodiments, R^{10.D} is independently unsubstituted cycloalkyl (e.g., C₃-C₈, C₃—C₆, or C₅—C₆). In embodiments, R^{10.D} is independently unsubstituted heterocycloalkyl (e.g., 3 to 8 membered, 3 to 6 membered, or 5 to 6 membered). In embodiments, R^{10.D} is independently unsubstituted aryl (e.g., C₆—C₁₀, C₁₀, or phenyl). In embodiments, R^{10.D} is independently unsubstituted heteroaryl (e.g., 5 to 10 membered, 5 to 9 membered, or 5 to 6 membered). In embodiments, R^{10.D} is independently —F. In embodiments, R^{10.D} is independently —Cl. In embodiments, R^{10.D} is independently —CH₃. In embodiments, R^{10.D} is independently —OCH₃. In embodiments, R^{10.D} is independently —OH. In embodiments, R^{10.D} is independently unsubstituted morpholinyl. In embodiments, R^{10.D} is independently unsubstituted piperazinyl. In embodiments, X^{10.D} is independently —F. In embodiments, X^{10.D} is independently —Cl. In embodiments, X^{10.D} is independently —Br. In embodiments, X^{10.D} is independently —I.

In embodiments, R^{10.E} is independently hydrogen, halogen, —CX^{10.E}₃, —CHX^{10.E}₂, —CH₂X^{10.E}, —OCX^{10.E}3, —OCH₂X^{10.E}, —OCHX^{10.E}2, —CN, —SOₙ₁₀R^{10D}, —SOᵥ₁₀NR^{10A}R^{10B}, —NR^{10C}CNR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆-C₁₀, C₁₀, or phenyl), or substituted or unsubstituted heteroaryl (e.g., 5 to 10 membered, 5 to 9 membered, or 5 to 6 membered),. X^{10.E} is independently halogen.

In embodiments, R^{10.E} is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R^{10.E} is independently hydrogen. In embodiments, R^{10.E} is independently halogen. In embodiments, R^{10.E} is independently —CX^{10.E}₃. In embodiments, R^{10.E} is independently —CHX^{10.E}₂. In embodiments, R^{10.E} is independently —CH₂X^{.E}. In embodiments, R^{10.E} is independently —OCX^{10.E}₃. In embodiments, R^{10.E} is independently —OCH₂X^{10.E}. In embodiments, R^{10.E} is independently —OCHX^{10.E}₂. In embodiments, R^{10.E} is independently —CN. In embodiments, R^{10.E} is independently —SOₙ₁₀R^{10D}. In embodiments, R^{10.E} is independently —SOᵥ₁₀NR^{10A}R^{10B}. In embodiments, R^{10.E} is independently —NR^{10C}NR^{10A}R^{10B}. In embodiments, R^{10.E} is independently —ONR^{10A}R^{10B}. In embodiments, R^{10.E} is independently —NHC(O)NR^{10C}NR^{10A}R^{10B}. In embodiments, R^{10.E} is independently —NHC(O)NR^{10A}R^{10B}. In embodiments, R^{10.E} is independently -N(O)ₘ₁₀. In embodiments, R^{10.E} is independently -NR^{10A}R^{10B}. In embodiments, R^{10.E} is independently —C(O)R^{10C}. In embodiments, R^{10.E} is independently —C(O)—OR^{10C}. In embodiments, R^{10.E} is independently —C(O)NR^{10A}R^{10B}. In embodiments, R^{10.E} is independently -OR^{10D}. In embodiments, R^{10.E} is independently —NR^{10A}SO₂R^{10D}. In embodiments, R^{10.E} is independently —NR^{10A}C(O)R^{10C}. In embodiments, R^{10.E} is independently —NR^{10A}C(O)OR^{10C}. In embodiments, R^{10.E} is independently —NR^{10A}OR^{10C}. In embodiments, R^{10.E} is independently —SF₅. In embodiments, R^{10.E} is independently —N₃. In embodiments, R^{10.E} is independently —F. In embodiments, R^{10.E} is independently —Cl. In embodiments, R^{10.E} is independently —Br. In embodiments, R^{10.E} is independently —I. In embodiments, R^{10.E} is independently —CH₂OCH₃. In embodiments, R^{10.E} is independently —SO₂CH₃. In embodiments, R^{10.E} is independently —SCH₃. In embodiments, R^{10.E} is independently —OCH₃. In embodiments, R^{10.E} is independently —CH₂CH₂OCH₃. In embodiments, R^{10.E} is independently —SO₂CH₂CH₃. In embodiments, R^{10.E} is independently —SCH2CH3. In embodiments, R^{10.E} is independently —OCH2CH3. In embodiments, R^{10.E} is independently —CH₂OCH₂CH₃. In embodiments, R^{10.E} is independently unsubstituted C₁—C₄ alkyl. In embodiments, R^{10.E} is independently unsubstituted cyclopropyl. In embodiments, R^{10.E} is independently unsubstituted phenyl. In embodiments, R^{10.E} is independently hydrogen. In embodiments, R^{10.E} is independently —CCl₃. In embodiments, R^{10.E} is independently —CBr₃. In embodiments, R^{10.E} is independently —CF₃. In embodiments, R^{10.E} is independently —CI₃. In embodiments, R^{10.E} is independently —CHCl₂. In embodiments, R^{10.E} is independently —CHBr₂. In embodiments, R^{10.E} is independently —CHF₂. In embodiments, R^{10.E} is independently —CHI₂. In embodiments, R^{10.E} is independently —CH₂Cl. In embodiments, R^{10.E} is independently —CH₂Br. In embodiments, R^{10.E} is independently —CH₂F. In embodiments, R^{10.E} is independently —CH₂I. In embodiments, R^{10.E} is independently —CN. In embodiments, R^{10.E} is independently —OH. In embodiments, R^{10.E} is independently —NH₂. In embodiments, R^{10.E} is independently —COOH. In embodiments, R^{10.E} is independently —CONH₂. In embodiments, R^{10.E} is independently —OCCl₃. In embodiments, R^{10.E} is independently —OCF₃. In embodiments, R^{10.E} is independently —OCBr₃. In embodiments, R^{10.E} is independently —OCI₃. In embodiments, R^{10.E} is independently —OCHCl₂. In embodiments, R^{10.E} is independently —OCHBr₂. In embodiments, R^{10.E} is independently —OCHI₂. In embodiments, R^{10.E} is independently —OCHF₂. In embodiments, R^{10.E} is independently —OCH₂Cl. In embodiments, R^{10.E} is independently —OCH₂Br. In embodiments, R^{10.E} is independently —OCH₂I. In embodiments, R^{10.E} is independently —OCH₂F. In embodiments, R^{10.E} is independently halogen. In embodiments, R^{10.E} is independently —NO₂. In embodiments, R^{10.E} is independently —OCH₃. In embodiments, R^{10.E} is independently —OCH₂CH₃. In embodiments, R^{10.E} is independently —OCH(CH₃)₂. In embodiments, R^{10.E} is independently —OC(CH₃)₃. In embodiments, R^{10.E} is independently —CH₃. In embodiments, R^{10.E} is independently —CH₂CH₃. In embodiments, R^{10.E} is independently —CH(CH₃)₂. In embodiments, R^{10.E} is independently —C(CH₃)₃. In embodiments, R^{10.E} is independently unsubstituted cyclopropyl. In embodiments, R^{10.E} is independently unsubstituted cyclobutyl. In embodiments, R^{10.E} is independently unsubstituted cyclopentyl. In embodiments, R^{10.E} is independently unsubstituted cyclohexyl. In embodiments, R^{10.E} is independently substituted or unsubstituted alkyl (e.g., C₁—C₈, C₁—C₆, or C₁—C₄). In embodiments, R^{10.E} is independently substituted or unsubstituted heteroalkyl (e.g., 2 to 8 membered, 2 to 6 membered, or 2 to 4 membered). In embodiments, R^{10.E} is independently substituted or unsubstituted cycloalkyl (e.g., C₃—C₈, C₃—C₆, or C₅—C₆). In embodiments, R^{10.E} is independently substituted or unsubstituted heterocycloalkyl (e.g., 3 to 8 membered, 3 to 6 membered, or 5 to 6 membered). In embodiments, R^{10.E} is independently substituted or unsubstituted aryl (e.g., C₆—C₁₀, C₁₀, or phenyl). In embodiments, R^{10.E} is independently substituted or unsubstituted heteroaryl (e.g., 5 to 10 membered, 5 to 9 membered, or 5 to 6 membered). In embodiments, R^{10.E} is independently unsubstituted alkyl (e.g., C₁—C₈, C₁—C₆, or C₁—C₄). In embodiments, R^{10.E} is independently unsubstituted heteroalkyl (e.g., 2 to 8 membered, 2 to 6 membered, or 2 to 4 membered). In embodiments, R^{10.E} is independently unsubstituted cycloalkyl (e.g., C₃—C₈, C₃—C₆, or C₅—C₆). In embodiments, R^{10.E} is independently unsubstituted heterocycloalkyl (e.g., 3 to 8 membered, 3 to 6 membered, or 5 to 6 membered). In embodiments, R^{10.E} is independently unsubstituted aryl (e.g., C₆—C₁₀, C₁₀, or phenyl). In embodiments, R^{10.E} is independently unsubstituted heteroaryl (e.g., 5 to 10 membered, 5 to 9 membered, or 5 to 6 membered). In embodiments, R^{10.E} is independently —F. In embodiments, R^{10.E} is independently —Cl. In embodiments, R^{10.E} is independently —CH₃. In embodiments, R^{10.E} is independently —OCH₃. In embodiments, R^{10.E} is independently —OH. In embodiments, R^{10.E} is independently unsubstituted morpholinyl. In embodiments, R^{10.E} is independently unsubstituted piperazinyl. In embodiments, X^{10.E} is independently —F. In embodiments, X^{10.E} is independently —Cl. In embodiments, X^{10.E} is independently —Br. In embodiments, X^{10.E} is independently —I.

In embodiments, L² is a bond. In embodiments, L² is —N(R^{L2})—. In embodiments, L² is —O—. In embodiments, L² is —S—. In embodiments, L² is —SO₂—. In embodiments, L² is —C(O)—. In embodiments, L² is —C(O)N(R^{L2})—. In embodiments, L² is —N(R^{L2})C(O)—. In embodiments, L² is —N(R^{L2})C(O)NH—. In embodiments, L² is —NHC(O)N(R^{L2})—. In embodiments, L² is —C(O)O—. In embodiments, L² is —OC(O)—. In embodiments, L² is —SO₂N(R^{L2})—. In embodiments, L² is —N(R^{L2})SO₂—. In embodiments, L² is substituted or unsubstituted alkylene (e.g., C₁—C₈, C₁—C₆, or C₁—C₄). In embodiments, L² is substituted or unsubstituted heteroalkylene (e.g., 2 to 8 membered, 2 to 6 membered, or 2 to 4 membered). In embodiments, L² is a substituted or unsubstituted 2 to 6 membered heteroalkylene.

In embodiments, L² is a bond. In embodiments, L² is —N(R^{L2})—. In embodiments, L² is —O—. In embodiments, L² is —S—. In embodiments, L² is —SO₂—. In embodiments, L² is —C(O)—. In embodiments, L² is —C(O)N(R^{L2})—. In embodiments, L² is —N(R^{L2})C(O)—. In embodiments, L² is —N(R^{L2})C(O)NH—. In embodiments, L² is —NHC(O)N(R^{L2})—. In embodiments, L² is —C(O)O—. In embodiments, L² is —OC(O)—. In embodiments, L² is —SO₂N(R^{L2})—. In embodiments, L² is —N(R^{L2})SO₂—.

In embodiments, L² is a bond or substituted or unsubstituted C₁—C₆ alkylene. In embodiments, L² is a bond or unsubstituted C₁—C₄ alkylene. In embodiments, L² is a bond. In embodiments, L² is unsubstituted C₁—C₄ alkylene. In embodiments, L² is unsubstituted methylene. In embodiments, L² is unsubstituted ethylene. In embodiments, L² is unsubstituted propylene. In embodiments, L² is unsubstituted butylene. In embodiments, L² is unsubstituted n-butylene. In embodiments, L² is unsubstituted tert-butylene. In embodiments, L² is unsubstituted iso-butylene. In embodiments, L² is unsubstituted sec-butylene.

In embodiments, R^{L2} is independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, unsubstituted alkyl, or unsubstituted cycloalkyl. In embodiments, R^{L2} is independently hydrogen, unsubstituted C₁—C₆ alkyl, or unsubstituted C₃—C₆ cycloalkyl. In embodiments, R^{L2} is independently hydrogen, unsubstituted methyl, unsubstituted ethyl, unsubstituted isopropyl, or unsubstituted cyclopropyl. In embodiments, R^{L2} is independently hydrogen. In embodiments, R^{L2} is independently unsubstituted methyl. In embodiments, R^{L2} is independently unsubstituted ethyl. In embodiments, R^{L2} is independently unsubstituted isopropyl. In embodiments, R^{L2} is independently unsubstituted cyclopropyl.

In embodiments, R² is independently hydrogen, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R² is independently substituted or unsubstituted C₁—C₄ alkyl or substituted or unsubstituted C₃—C₆ cycloalkyl. In embodiments, R² is independently unsubstituted C₁—C₄ alkyl or unsubstituted C₃—C₆ cycloalkyl. In embodiments, R² is independently unsubstituted methyl or unsubstituted cyclopropyl. In embodiments, R² is independently unsubstituted methyl.

In embodiments, R² is independently hydrogen. In embodiments, R² is independently halogen. In embodiments, R² is independently —CX²₃. In embodiments, R² is independently —CHX²₂. In embodiments, R² is independently —CH₂X². In embodiments, R² is independently —OCX²₃. In embodiments, R² is independently —OCH₂X². In embodiments, R² is independently —OCHX²₂. In embodiments, R² is independently —CN. In embodiments, R² is independently —SF₅. In embodiments, R² is independently —N₃. In embodiments, R² is independently —SOₙ₂R^{2D}. In embodiments, R² is independently —SOᵥ₂NR^{2A}R^{2B}. In embodiments, R² is independently —NR^{2C}NR^{2A}R^{2B}. In embodiments, R² is independently —ONR^{2A}R^{2B}. In embodiments, R² is independently —NHC(O)NR^{2C}NR^{2A}R^{2B}. In embodiments, R² is independently —NHC(O)NR^{2A}R^{2B}. In embodiments, R² is independently —N(O)ₘ₂. In embodiments, R² is independently —NR^{2A}R^{2B}. In embodiments, R² is independently —C(O)R^{2C}. In embodiments, R² is independently —C(O)—OR^{2C}. In embodiments, R² is independently —C(O)NR^{2A}R^{2B}. In embodiments, R² is independently —OR^{2D}. In embodiments, R² is independently —NR^{2A}SO₂R^{2D}. In embodiments, R² is independently —NR^{2A}C(O)R^{2C}. In embodiments, R² is independently —NR^{2A}C(O)OR^{2C}. In embodiments, R² is independently -NR^{2A}OR^{2C}.

In embodiments, R² is independently —F. In embodiments, R² is independently —Cl. In embodiments, R² is independently —Br. In embodiments, R² is independently —I. In embodiments, R² is independently —SCH₃. In embodiments, R² is independently —OCH₃. In embodiments, R² is independently —SCH₂CH₃. In embodiments, R² is independently —OCH₂CH₃. In embodiments, R² is independently unsubstituted C₁—C₄ alkyl. In embodiments, R² is independently unsubstituted cyclopropyl. In embodiments, R² is independently hydrogen. In embodiments, R² is independently —CCl₃. In embodiments, R² is independently —CBr₃. In embodiments, R² is independently —CF₃. In embodiments, R² is independently —CI₃. In embodiments, R² is independently —CHCl₂. In embodiments, R² is independently —CHBr₂. In embodiments, R² is independently —CHF₂. In embodiments, R² is independently —CHI2. In embodiments, R² is independently —CH₂Cl. In embodiments, R² is independently —CH2Br. In embodiments, R² is independently —CH₂F. In embodiments, R² is independently —CH₂I. In embodiments, R² is independently —CN. In embodiments, R² is independently —OH. In embodiments, R² is independently —NH₂. In embodiments, R² is independently —COOH. In embodiments, R² is independently —CONH₂. In embodiments, R² is independently —OCCl₃. In embodiments, R² is independently —OCF₃. In embodiments, R² is independently —OCBr₃. In embodiments, R² is independently —OCI₃. In embodiments, R² is independently —OCHCl₂. In embodiments, R² is independently —OCHBr₂. In embodiments, R² is independently —OCHI2. In embodiments, R² is independently —OCHF₂. In embodiments, R² is independently —OCH₂Cl. In embodiments, R² is independently —OCH₂Br. In embodiments, R² is independently —OCH₂I. In embodiments, R² is independently —OCH₂F. In embodiments, R² is independently —OCH₃. In embodiments, R² is independently -OCH₂CH₃. In embodiments, R² is independently —OCH(CH₃)₂. In embodiments, R² is independently —OC(CH₃)₃. In embodiments, R² is independently —CH₃. In embodiments, R² is independently —CH₂CH₃. In embodiments, R² is independently —CH(CH₃)₂. In embodiments, R² is independently —C(CH₃)₃. In embodiments, R² is independently unsubstituted cyclopropyl. In embodiments, R² is independently unsubstituted cyclobutyl. In embodiments, R² is independently unsubstituted cyclopentyl. In embodiments, R² is independently unsubstituted cyclohexyl. In embodiments, R² is independently halogen. In embodiments, R² is independently —NO₂. In embodiments, R² is independently —CH₂CH(CH₃)₂. In embodiments, R² is independently unsubstituted propyl. In embodiments, R² is independently unsubstituted butyl. In embodiments, R² is independently unsubstituted pentyl. In embodiments, R² is independently unsubstituted hexyl. In embodiments, R² is independently substituted or unsubstituted alkyl (e.g., C₁—C₈, C₁—C₆, or C₁—C₄). In embodiments, R² is independently substituted or unsubstituted heteroalkyl (e.g., 2 to 8 membered, 2 to 6 membered, or 2 to 4 membered). In embodiments, R² is independently substituted or unsubstituted cycloalkyl (e.g., C₃—C₈, C₃—C₆, or C₅—C₆). In embodiments, R² is independently substituted or unsubstituted heterocycloalkyl (e.g., 3 to 8 membered, 3 to 6 membered, or 5 to 6 membered). In embodiments, R² is independently substituted or unsubstituted aryl (e.g., C₆—C₁₀, C₁₀, or phenyl). In embodiments, R² is independently substituted or unsubstituted heteroaryl (e.g., 5 to 10 membered, 5 to 9 membered, or 5 to 6 membered). In embodiments, R² is independently unsubstituted alkyl (e.g., C₁—C₈, C₁—C₆, or C₁—C₄). In embodiments, R² is independently unsubstituted heteroalkyl (e.g., 2 to 8 membered, 2 to 6 membered, or 2 to 4 membered). In embodiments, R² is independently unsubstituted cycloalkyl (e.g., C₃—C₈, C₃—C₆, or C₅—C₆). In embodiments, R² is independently unsubstituted heterocycloalkyl (e.g., 3 to 8 membered, 3 to 6 membered, or 5 to 6 membered). In embodiments, R² is independently unsubstituted aryl (e.g., C₆—C₁₀, C₁₀, or phenyl). In embodiments, R² is independently unsubstituted heteroaryl (e.g., 5 to 10 membered, 5 to 9 membered, or 5 to 6 membered).

In embodiments, R² is independently substituted or unsubstituted aryl or substituted or unsubstituted heteroaryl. In embodiments, R² is independently substituted phenyl or substituted 5 to 6 membered heteroaryl.

In embodiments, R² is independently substituted or unsubstituted C₁—C₆ alkyl, substituted or unsubstituted 2 to 6 membered heteroalkyl, substituted or unsubstituted C₃—C₆ cycloalkyl, substituted or unsubstituted 3 to 6 membered heterocycloalkyl, substituted or unsubstituted phenyl, or substituted or unsubstituted 5 to 6 membered heteroaryl.

In embodiments, R² is independently unsubstituted alkyl. In embodiments, R² is independently substituted or unsubstituted aryl or substituted or unsubstituted heteroaryl. In embodiments, R² is independently substituted or unsubstituted phenyl or substituted or unsubstituted 5 to 6 membered heteroaryl. In embodiments, R² is independently substituted or unsubstituted phenyl or substituted or unsubstituted 5 to 6 membered heteroaryl. In embodiments, R² is independently substituted or unsubstituted phenyl. In embodiments, R² is independently substituted or unsubstituted 5 to 6 membered heteroaryl. In embodiments, R² is independently substituted phenyl. In embodiments, R² is independently substituted 5 to 6 membered heteroaryl. In embodiments, R² is independently unsubstituted phenyl. In embodiments, R² is independently unsubstituted 5 to 6 membered heteroaryl. In embodiments, R² is independently substituted 5 membered heteroaryl. In embodiments, R² is independently substituted 6 membered heteroaryl. In embodiments, R² is independently unsubstituted 5 membered heteroaryl. In embodiments, R² is independently unsubstituted 6 membered heteroaryl.

In embodiments, R² is substituted or unsubstituted 5 membered heteroaryl. In embodiments, R² is substituted or unsubstituted triazolyl. In embodiments, R² is substituted or unsubstituted 1,2,4-triazolyl. In embodiments, R² is substituted or unsubstituted pyrrolyl. In embodiments, R² is substituted or unsubstituted pyrazolyl. In embodiments, R² is substituted or unsubstituted imidazolyl. In embodiments, R² is substituted or unsubstituted tetrazolyl. In embodiments, R² is substituted or unsubstituted furanyl. In embodiments, R² is substituted or unsubstituted thienyl. In embodiments, R² is substituted or unsubstituted oxazolyl. In embodiments, R² is substituted or unsubstituted isoxazolyl. In embodiments, R² is substituted or unsubstituted thiazolyl. In embodiments, R² is substituted or unsubstituted isothiazolyl. In embodiments, R² is substituted or unsubstituted oxadiazolyl. In embodiments, R² is substituted or unsubstituted thiadiazolyl. In embodiments, R² is unsubstituted 5 membered heteroaryl. In embodiments, R² is unsubstituted triazolyl. In embodiments, R² is unsubstituted 1,2,4-triazolyl. In embodiments, R² is unsubstituted pyrrolyl. In embodiments, R² is unsubstituted pyrazolyl. In embodiments, R² is unsubstituted imidazolyl. In embodiments, R² is unsubstituted tetrazolyl. In embodiments, R² is unsubstituted furanyl. In embodiments, R² is unsubstituted thienyl. In embodiments, R² is unsubstituted oxazolyl. In embodiments, R² is unsubstituted isoxazolyl. In embodiments, R² is unsubstituted thiazolyl. In embodiments, R² is unsubstituted isothiazolyl. In embodiments, R² is unsubstituted oxadiazolyl. In embodiments, R² is unsubstituted thiadiazolyl.

In embodiments, R² is substituted or unsubstituted C₃—C₆ cycloalkyl. In embodiments, R² is substituted or unsubstituted C₄—C₆ cycloalkyl. In embodiments, R² is substituted or unsubstituted C₅—C₆ cycloalkyl. In embodiments, R² is substituted or unsubstituted C₃ cycloalkyl. In embodiments, R² is substituted or unsubstituted C₄ cycloalkyl. In embodiments, R² is substituted or unsubstituted C₅ cycloalkyl. In embodiments, R² is substituted or unsubstituted C₆ cycloalkyl. In embodiments, R² is substituted or unsubstituted C₃ cycloalkenyl. In embodiments, R² is substituted or unsubstituted C₄ cycloalkenyl. In embodiments, R² is substituted or unsubstituted C₅ cycloalkenyl. In embodiments, R² is substituted or unsubstituted C₆ cycloalkenyl.

In embodiments, R² is substituted or unsubstituted 3 to 6 membered heterocycloalkyl. In embodiments, R² is substituted or unsubstituted 4 to 6 membered heterocycloalkyl. In embodiments, R² is substituted or unsubstituted 5 to 6 membered heterocycloalkyl. In embodiments, R² is substituted or unsubstituted 3 membered heterocycloalkyl. In embodiments, R² is substituted or unsubstituted 4 membered heterocycloalkyl. In embodiments, R² is substituted or unsubstituted 5 membered heterocycloalkyl. In embodiments, R² is substituted or unsubstituted 6 membered heterocycloalkyl. In embodiments, R² is substituted or unsubstituted 3 membered heterocycloalkenyl. In embodiments, R² is substituted or unsubstituted 4 membered heterocycloalkenyl. In embodiments, R² is substituted or unsubstituted 5 membered heterocycloalkenyl. In embodiments, R² is substituted or unsubstituted 6 membered heterocycloalkenyl.

In embodiments, R² is substituted or unsubstituted phenyl. In embodiments, R² is substituted or unsubstituted 5 to 6 membered heteroaryl. In embodiments, R² is substituted or unsubstituted 6 membered heteroaryl. In embodiments, R² is unsubstituted phenyl. In embodiments, R² is unsubstituted 5 to 6 membered heteroaryl. In embodiments, R² is unsubstituted 6 membered heteroaryl.

In embodiments, R² is a bond or substituted or unsubstituted C₁—C₆ alkyl. In embodiments, R² is a bond or unsubstituted C₁—C₄ alkyl. In embodiments, R² is a bond. In embodiments, R² is unsubstituted C₁—C₄ alkyl. In embodiments, R² is unsubstituted methyl. In embodiments, R² is unsubstituted ethyl. In embodiments, R² is unsubstituted propyl. In embodiments, R² is unsubstituted n-propyl. In embodiments, R² is unsubstituted isopropyl. In embodiments, R² is unsubstituted butyl. In embodiments, R² is unsubstituted n-butyl. In embodiments, R² is unsubstituted tert-butyl. In embodiments, R² is unsubstituted iso-butyl. In embodiments, R² is unsubstituted sec-butyl.

In embodiments, R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, R²⁰-substituted or unsubstituted alkyl (e.g., C₁—C₈, C₁—C₆, or C₁—C₄), R²⁰-substituted or unsubstituted heteroalkyl (e.g., 2 to 8 membered, 2 to 6 membered, or 2 to 4 membered), R²⁰-substituted or unsubstituted cycloalkyl (e.g., C₃—C₈, C₃—C₆, or C₅—C₆), R²⁰-substituted or unsubstituted heterocycloalkyl (e.g., 3 to 8 membered, 3 to 6 membered, or 5 to 6 membered), R²⁰-substituted or unsubstituted aryl (e.g., C₆—C₁₀, C₁₀, or phenyl), or R²⁰-substituted or unsubstituted heteroaryl (e.g., 5 to 10 membered, 5 to 9 membered, or 5 to 6 membered).

In embodiments, R² is independently R²⁰-substituted or unsubstituted C₁—C₆ alkyl, R²⁰-substituted or unsubstituted 2 to 6 membered heteroalkyl, R²⁰-substituted or unsubstituted C₃—C₆ cycloalkyl, R²⁰-substituted or unsubstituted 3 to 6 membered heterocycloalkyl, R²⁰-substituted or unsubstituted phenyl, or R²⁰-substituted or unsubstituted 5 to 6 membered heteroaryl. In embodiments, R² is independently R²⁰-substituted or unsubstituted C₁—C₆ alkyl. In embodiments, R² is independently R²⁰-substituted or unsubstituted 2 to 6 membered heteroalkyl. In embodiments, R² is independently R²⁰-substituted or unsubstituted C₃—C₆ cycloalkyl. In embodiments, R² is independently R²⁰-substituted or unsubstituted 3 to 6 membered heterocycloalkyl. In embodiments, R² is independently R²⁰-substituted or unsubstituted phenyl. In embodiments, R² is independently or R²⁰-substituted or unsubstituted 5 to 6 membered heteroaryl.

In embodiments, R² is independently R²⁰-substituted or unsubstituted C₃—C₆ cycloalkyl, R²⁰-substituted or unsubstituted 3 to 6 membered heterocycloalkyl, R²⁰-substituted or unsubstituted phenyl, or R²⁰-substituted or unsubstituted 5 to 6 membered heteroaryl.

In embodiments, R² is independently R²⁰-substituted or unsubstituted alkyl (e.g., C₁-C₈, C₁—C₆, or C₁—C₄). In embodiments, R² is independently R²⁰-substituted or unsubstituted heteroalkyl (e.g., 2 to 8 membered, 2 to 6 membered, or 2 to 4 membered). In embodiments, R² is independently R²⁰-substituted or unsubstituted cycloalkyl (e.g., C₃—C₈, C₃—C₆, or C₅—C₆). In embodiments, R² is independently R²⁰-substituted or unsubstituted heterocycloalkyl (e.g., 3 to 8 membered, 3 to 6 membered, or 5 to 6 membered). In embodiments, R² is independently R²⁰-substituted or unsubstituted aryl (e.g., C₆—C₁₀, C₁₀, or phenyl). In embodiments, R² is independently R²⁰-substituted or unsubstituted heteroaryl (e.g., 5 to 10 membered, 5 to 9 membered, or 5 to 6 membered).

In embodiments, R² is independently R²⁰-substituted phenyl or R²⁰-substituted 5 to 6 membered heteroaryl. In embodiments, R² is independently R²⁰-substituted phenyl. In embodiments, R² is independently R²⁰-substituted 5 to 6 membered heteroaryl. In embodiments, R² is independently

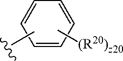

and R²⁰ is as described herein and z20 is independently an integer from 0 to 5. z20 is independently an integer from 0 to 9. In embodiments, z20 is independently 0. In embodiments, z20 is independently 1. In embodiments, z20 is independently 2. In embodiments, z20 is independently 3. In embodiments, z20 is independently 4. In embodiments, z20 is independently 5. In embodiments, z20 is independently 6. In embodiments, z20 is independently 7. In embodiments, z20 is independently 8. In embodiments, z20 is independently 9. In embodiments, z20 is independently an integer from 0 to 5. In embodiments, R² is independently

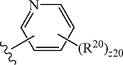

and R²⁰ is as described herein and z20 is independently an integer from 0 to 4. In embodiments, R² is independently

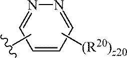

and R²⁰ is as described herein and z20 is independently an integer from 0 to 3. In embodiments, R² is independently

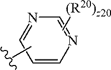

and R²⁰ is as described herein and z20 is independently an integer from 0 to 3. In embodiments, R² is independently

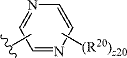

and R²⁰ is as described herein and z20 is independently an integer from 0 to 3. In embodiments, R² is independently

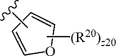

and R²⁰ is as described herein and z20 is independently an integer from 0 to 3.

In embodiments, R² is independently

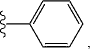

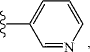

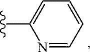

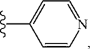

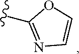

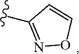

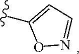

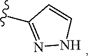

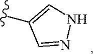

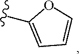

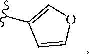

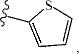

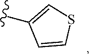

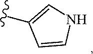

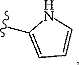

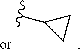

In embodiments, R² is independently

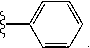

In embodiments, R² is independently

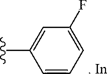

embodiments, R² is independently

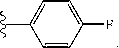

In embodiments, R² is independently

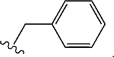

In embodiments, R² is independently

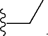

In embodiments, R² is independently

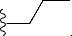

In embodiments, R² is independently

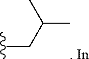

embodiments, R² is independently

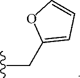

In embodiments, X² is independently —F. In embodiments, X² is independently —Cl. In embodiments, X² is independently —Br. In embodiments, X² is independently —I.

In embodiments, n2 is independently 0. In embodiments, n2 is independently 1. In embodiments, n2 is independently 2. In embodiments, n2 is independently 3. In embodiments, n2 is independently 4.

In embodiments, m2 is independently 1. In embodiments, m2 is independently 2. In embodiments, v2 is independently 1. In embodiments, v2 is independently 2.

In embodiments, R^{2A} is independently hydrogen. In embodiments, R^{2A} is independently —CCl₃. In embodiments, R^{2A} is independently —CBr₃. In embodiments, R^{2A} is independently —CF₃. In embodiments, R^{2A} is independently —CI₃. In embodiments, R^{2A} is independently —CHCl₂. In embodiments, R^{2A} is independently —CHBr2. In embodiments, R^{2A} is independently —CHF₂. In embodiments, R^{2A} is independently —CHI2. In embodiments, R^{2A} is independently —CH₂Cl. In embodiments, R^{2A} is independently —CH2Br. In embodiments, R^{2A} is independently —CH₂F. In embodiments, R^{2A} is independently —CH₂I. In embodiments, R^{2A} is independently —CN. In embodiments, R^{2A} is independently —OH. In embodiments, R^{2A} is independently —NH₂. In embodiments, R^{2A} is independently —COOH. In embodiments, R^{2A} is independently —CONH₂. In embodiments, R^{2A} is independently —OCCl₃. In embodiments, R^{2A} is independently —OCF₃. In embodiments, R^{2A} is independently —OCBr₃. In embodiments, R^{2A} is independently —OCI₃. In embodiments, R^{2A} is independently —OCHCl₂. In embodiments, R^{2A} is independently —OCHBr₂. In embodiments, R^{2A} is independently —OCHI2. In embodiments, R^{2A} is independently —OCHF₂. In embodiments, R^{2A} is independently —OCH₂Cl. In embodiments, R^{2A} is independently —OCH₂Br. In embodiments, R^{2A} is independently —OCH₂I. In embodiments, R^{2A} is independently —OCH₂F. In embodiments, R^{2A} is independently halogen. In embodiments, R^{2A} is independently —NO₂. In embodiments, R^{2A} is independently —OCH₃. In embodiments, R^{2A} is independently —OCH₂CH₃. In embodiments, R^{2A} is independently —OCH(CH₃)₂. In embodiments, R^{2A} is independently —OC(CH₃)₃. In embodiments, R^{2A} is independently —CH₃. In embodiments, R^{2A} is independently —CH₂CH₃. In embodiments, R^{2A} is independently —CH(CH₃)₂. In embodiments, R^{2A} is independently —C(CH₃)₃. In embodiments, R^{2A} is independently unsubstituted cyclopropyl. In embodiments, R^{2A} is independently unsubstituted cyclobutyl. In embodiments, R^{2A} is independently unsubstituted cyclopentyl. In embodiments, R^{2A} is independently unsubstituted cyclohexyl. In embodiments, R^{2A} is independently substituted or unsubstituted alkyl (e.g., C₁—C₈, C₁—C₆, or C₁—C₄). In embodiments, R^{2A} is independently substituted or unsubstituted heteroalkyl (e.g., 2 to 8 membered, 2 to 6 membered, or 2 to 4 membered). In embodiments, R^{2A} is independently substituted or unsubstituted cycloalkyl (e.g., C₃—C₈, C₃—C₆, or C₅—C₆). In embodiments, R^{2A} is independently substituted or unsubstituted heterocycloalkyl (e.g., 3 to 8 membered, 3 to 6 membered, or 5 to 6 membered). In embodiments, R^{2A} is independently substituted or unsubstituted aryl (e.g., C₆-C₁₀, C₁₀, or phenyl). In embodiments, R^{2A} is independently substituted or unsubstituted heteroaryl (e.g., 5 to 10 membered, 5 to 9 membered, or 5 to 6 membered).

In embodiments, R^{2B} is independently hydrogen. In embodiments, R^{2B} is independently —CCl₃. In embodiments, R^{2B} is independently —CBr₃. In embodiments, R^{2B} is independently —CF₃. In embodiments, R^{2B} is independently —CI₃. In embodiments, R^{2B} is independently —CHCl₂. In embodiments, R^{2B} is independently —CHBr2. In embodiments, R^{2B} is independently —CHF₂. In embodiments, R^{2B} is independently —CHI2. In embodiments, R^{2B} is independently —CH₂Cl. In embodiments, R^{2B} is independently —CH2Br. In embodiments, R^{2B} is independently —CH₂F. In embodiments, R^{2B} is independently —CH₂I. In embodiments, R^{2B} is independently —CN. In embodiments, R^{2B} is independently —OH. In embodiments, R^{2B} is independently —NH₂. In embodiments, R^{2B} is independently -COOH. In embodiments, R^{2B} is independently —CONH₂. In embodiments, R^{2B} is independently —OCCl₃. In embodiments, R^{2B} is independently —OCF₃. In embodiments, R^{2B} is independently —OCBr₃. In embodiments, R^{2B} is independently —OCI₃. In embodiments, R^{2B} is independently —OCHCl₂. In embodiments, R^{2B} is independently —OCHBr₂. In embodiments, R^{2B} is independently —OCHI2. In embodiments, R^{2B} is independently —OCHF₂. In embodiments, R^{2B} is independently —OCH₂Cl. In embodiments, R^{2B} is independently —OCH₂Br. In embodiments, R^{2B} is independently —OCH₂I. In embodiments, R^{2B} is independently —OCH₂F. In embodiments, R^{2B} is independently halogen. In embodiments, R^{2B} is independently —NO₂. In embodiments, R^{2B} is independently —OCH₃. In embodiments, R^{2B} is independently —OCH₂CH₃. In embodiments, R^{2B} is independently —OCH(CH₃)₂. In embodiments, R^{2B} is independently —OC(CH₃)₃. In embodiments, R^{2B} is independently —CH₃. In embodiments, R^{2B} is independently —CH₂CH₃. In embodiments, R^{2B} is independently —CH(CH₃)₂. In embodiments, R^{2B} is independently —C(CH₃)₃. In embodiments, R^{2B} is independently unsubstituted cyclopropyl. In embodiments, R^{2B} is independently unsubstituted cyclobutyl. In embodiments, R^{2B} is independently unsubstituted cyclopentyl. In embodiments, R^{2B} is independently unsubstituted cyclohexyl. In embodiments, R^{2B} is independently substituted or unsubstituted alkyl (e.g., C₁—C₈, C₁—C₆, or C₁—C₄). In embodiments, R^{2B} is independently substituted or unsubstituted heteroalkyl (e.g., 2 to 8 membered, 2 to 6 membered, or 2 to 4 membered). In embodiments, R^{2B} is independently substituted or unsubstituted cycloalkyl (e.g., C₃—C₈, C₃—C₆, or C₅—C₆). In embodiments, R^{2B} is independently substituted or unsubstituted heterocycloalkyl (e.g., 3 to 8 membered, 3 to 6 membered, or 5 to 6 membered). In embodiments, R^{2B} is independently substituted or unsubstituted aryl (e.g., C₆-C₁₀, C₁₀, or phenyl). In embodiments, R^{2B} is independently substituted or unsubstituted heteroaryl (e.g., 5 to 10 membered, 5 to 9 membered, or 5 to 6 membered).

In embodiments, R^{2A} and R^{2B} substituents bonded to the same nitrogen atom are joined to form a substituted or unsubstituted heterocycloalkyl (e.g., 3 to 8 membered, 3 to 6 membered, or 5 to 6 membered). In embodiments, R^{2A} and R^{2B} substituents bonded to the same nitrogen atom are joined to form a substituted or unsubstituted C₃—C₆ heterocycloalkyl. In embodiments, R^{2A} and R^{2B} bonded to the same nitrogen atom are joined to form a substituted or unsubstituted piperazinyl. In embodiments, R^{2A} and R^{2B} substituents bonded to the same nitrogen atom are joined to form a substituted or unsubstituted heteroaryl (e.g., 5 to 10 membered, 5 to 9 membered, or 5 to 6 membered). In embodiments, R^{2A} and R^{2B} substituents bonded to the same nitrogen atom are joined to form a substituted or unsubstituted 5 to 6 membered heteroaryl.

In embodiments, R^{2C} is independently hydrogen. In embodiments, R^{2C} is independently —CCl₃. In embodiments, R^{2C} is independently —CBr₃. In embodiments, R^{2C} is independently —CF₃. In embodiments, R^{2C} is independently —CI₃. In embodiments, R^{2C} is independently —CHCl₂. In embodiments, R^{2C} is independently —CHBr₂. In embodiments, R^{2C} is independently —CHF₂. In embodiments, R^{2C} is independently —CHI2. In embodiments, R^{2C} is independently —CH₂Cl. In embodiments, R^{2C} is independently —CH2Br. In embodiments, R^{2C} is independently —CH₂F. In embodiments, R^{2C} is independently —CH₂I. In embodiments, R^{2C} is independently —CN. In embodiments, R^{2C} is independently —OH. In embodiments, R^{2C} is independently —NH₂. In embodiments, R^{2C} is independently —COOH. In embodiments, R^{2C} is independently —CONH₂. In embodiments, R^{2C} is independently —OCCl₃. In embodiments, R^{2C} is independently —OCF₃. In embodiments, R^{2C} is independently —OCBr₃. In embodiments, R^{2C} is independently —OCI₃. In embodiments, R^{2C} is independently —OCHCl₂. In embodiments, R^{2C} is independently —OCHBr₂. In embodiments, R^{2C} is independently —OCHI2. In embodiments, R^{2C} is independently —OCHF₂. In embodiments, R^{2C} is independently —OCH₂Cl. In embodiments, R^{2C} is independently —OCH2Br. In embodiments, R^{2C} is independently —OCH₂I. In embodiments, R^{2C} is independently —OCH₂F. In embodiments, R^{2C} is independently halogen. In embodiments, R^{2C} is independently —NO₂. In embodiments, R^{2C} is independently —OCH₃. In embodiments, R^{2C} is independently —OCH₂CH₃. In embodiments, R^{2C} is independently —OCH(CH₃)₂. In embodiments, R^{2C} is independently —OC(CH₃)₃. In embodiments, R^{2C} is independently —CH₃. In embodiments, R^{2C} is independently —CH₂CH₃. In embodiments, R^{2C} is independently —CH(CH₃)₂. In embodiments, R^{2C} is independently —C(CH₃)₃. In embodiments, R^{2C} is independently unsubstituted cyclopropyl. In embodiments, R^{2C} is independently unsubstituted cyclobutyl. In embodiments, R^{2C} is independently unsubstituted cyclopentyl. In embodiments, R^{2C} is independently unsubstituted cyclohexyl. In embodiments, R^{2C} is independently substituted or unsubstituted alkyl (e.g., C₁—C₈, C₁—C₆, or C₁—C₄). In embodiments, R^{2C} is independently substituted or unsubstituted heteroalkyl (e.g., 2 to 8 membered, 2 to 6 membered, or 2 to 4 membered). In embodiments, R^{2C} is independently substituted or unsubstituted cycloalkyl (e.g., C₃—C₈, C₃—C₆, or C₅—C₆). In embodiments, R^{2C} is independently substituted or unsubstituted heterocycloalkyl (e.g., 3 to 8 membered, 3 to 6 membered, or 5 to 6 membered). In embodiments, R^{2C} is independently substituted or unsubstituted aryl (e.g., C₆-C₁₀, C₁₀, or phenyl). In embodiments, R^{2C} is independently substituted or unsubstituted heteroaryl (e.g., 5 to 10 membered, 5 to 9 membered, or 5 to 6 membered).

In embodiments, R^{2D} is independently hydrogen. In embodiments, R^{2D} is independently —CCl₃. In embodiments, R^{2D} is independently —CBr₃. In embodiments, R^{2D} is independently —CF₃. In embodiments, R^{2D} is independently —CI₃. In embodiments, R^{2D} is independently —CHCl₂. In embodiments, R^{2D} is independently —CHBr₂. In embodiments, R^{2D} is independently —CHF₂. In embodiments, R^{2D} is independently —CHI2. In embodiments, R^{2D} is independently —CH₂Cl. In embodiments, R^{2D} is independently —CH2Br. In embodiments, R^{2D} is independently —CH₂F. In embodiments, R^{2D} is independently —CH₂I. In embodiments, R^{2D} is independently —CN. In embodiments, R^{2D} is independently —OH. In embodiments, R^{2D} is independently —NH₂. In embodiments, R^{2D} is independently —COOH. In embodiments, R^{2D} is independently —CONH₂. In embodiments, R^{2D} is independently —OCCl₃. In embodiments, R^{2D} is independently —OCF₃. In embodiments, R^{2D} is independently —OCBr₃. In embodiments, R^{2D} is independently —OCI₃. In embodiments, R^{2D} is independently —OCHCl₂. In embodiments, R^{2D} is independently —OCHBr₂. In embodiments, R^{2D} is independently —OCHI2. In embodiments, R^{2D} is independently —OCHF₂. In embodiments, R^{2D} is independently —OCH₂Cl. In embodiments, R^{2D} is independently —OCH2Br. In embodiments, R^{2D} is independently —OCH₂I. In embodiments, R^{2D} is independently —OCH₂F. In embodiments, R^{2D} is independently halogen. In embodiments, R^{2D} is independently —NO₂. In embodiments, R^{2D} is independently —OCH₃. In embodiments, R^{2D} is independently —OCH₂CH₃. In embodiments, R^{2D} is independently —OCH(CH₃)₂. In embodiments, R^{2D} is independently —OC(CH₃)₃. In embodiments, R^{2D} is independently —CH₃. In embodiments, R^{2D} is independently —CH₂CH₃. In embodiments, R^{2D} is independently —CH(CH₃)₂. In embodiments, R^{2D} is independently —C(CH₃)₃. In embodiments, R^{2D} is independently unsubstituted cyclopropyl. In embodiments, R^{2D} is independently unsubstituted cyclobutyl. In embodiments, R^{2D} is independently unsubstituted cyclopentyl. In embodiments, R^{2D} is independently unsubstituted cyclohexyl. In embodiments, R^{2D} is independently substituted or unsubstituted alkyl (e.g., C₁—C₈, C₁—C₆, or C₁—C₄). In embodiments, R^{2D} is independently substituted or unsubstituted heteroalkyl (e.g., 2 to 8 membered, 2 to 6 membered, or 2 to 4 membered). In embodiments, R^{2D} is independently substituted or unsubstituted cycloalkyl (e.g., C₃—C₈, C₃—C₆, or C₅—C₆). In embodiments, R^{2D} is independently substituted or unsubstituted heterocycloalkyl (e.g., 3 to 8 membered, 3 to 6 membered, or 5 to 6 membered). In embodiments, R^{2D} is independently substituted or unsubstituted aryl (e.g., C₆-C₁₀, C₁₀, or phenyl). In embodiments, R^{2D} is independently substituted or unsubstituted heteroaryl (e.g., 5 to 10 membered, 5 to 9 membered, or 5 to 6 membered).

R²⁰ is independently oxo, halogen, -CX²⁰₃, -CHX²⁰₂, —CH₂X²⁰, -OCX²⁰₃, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R^{20B}, —N(O)ₘ₂₀, —NR^{20A}R^{20B}, —C(O)R^{20C}, —C(O)—OR^{20C}, —C(O)NR^{20A}R^{20B}, —OR^{20D}, —NR^{20A}SO₂R^{20D},—NR^{20A}C(O)R^{20C}, —NR^{20A}C(O)OR^{20C}, —NR^{20A}OR^{20C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); two adjacent R²⁰ substituents may optionally be joined to form a substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

X²⁰ is independently —F, —Cl, —Br, or —I. In embodiments, X²⁰ is independently —F. In embodiments, X²⁰ is independently —Cl. In embodiments, X²⁰ is independently —Br. In embodiments, X²⁰ is independently —I.

n20 is independently an integer from 0 to 4. In embodiments, n20 is independently 0. In embodiments, n20 is independently 1. In embodiments, n20 is independently 2. In embodiments, n20 is independently 3. In embodiments, n20 is independently 4.

m20 and v20 are independently 1 or 2. In embodiments, m20 is independently 1. In embodiments, m20 is independently 2. In embodiments, v20 is independently 1. In embodiments, v20 is independently 2.

In embodiments, R²⁰ is independently halogen, —CX²⁰₃, —CHX²⁰₂, —CH₂X²⁰, —OCX²⁰₃, —OCH₂X²⁰, —OCHX²⁰₂, —CN, —SOₙ₂₀R^{20D}, —SO_{V20}NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R^{20B}, —N(O)ₘ₂₀, —NR^{20A}R^{20B}, —C(O)R^{20C}, —C(O)—OR^{20C}, —C(O)NR^{20A}R^{20B}, —OR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); two adjacent R²⁰ substituents may optionally be joined to form a substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R²⁰ is independently halogen. In embodiments, R²⁰ is independently —F. In embodiments, R²⁰ is independently —Cl.

In embodiments, R²⁰ is independently substituted or unsubstituted C₁—C₆ alkyl. In embodiments, R²⁰ is independently substituted or unsubstituted 2 to 6 membered heteroalkyl. In embodiments, R²⁰ is independently substituted or unsubstituted C₃—C₆ cycloalkyl. In embodiments, R²⁰ is independently substituted or unsubstituted 3 to 6 membered heterocycloalkyl. In embodiments, R²⁰ is independently substituted or unsubstituted phenyl. In embodiments, R²⁰ is independently substituted or unsubstituted 5 to 6 membered heteroaryl.

In embodiments, R²⁰ is independently oxo, halogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered).

In embodiments, R²⁰ is independently halogen. In embodiments, R²⁰ is independently —F. In embodiments, R²⁰ is independently —Cl. In embodiments, R²⁰ is independently —Br. In embodiments, R²⁰ is independently —I. In embodiments, R²⁰ is independently oxo. In embodiments, R²⁰ is independently -CX²⁰₃. In embodiments, R²⁰ is independently —CHX²⁰₂. In embodiments, R²⁰ is independently —CH₂X²⁰. In embodiments, R²⁰ is independently -OCX²⁰₃. In embodiments, R²⁰ is independently —OCH₂X²⁰. In embodiments, R²⁰ is independently -OCHX²⁰₂. In embodiments, R²⁰ is independently —CN. In embodiments, R²⁰ is independently -SOₙ₁₀R^{20D}. In embodiments, R²⁰ is independently —SOᵥ₁₀NR²⁰AR^{20B}. In embodiments, R²⁰ is independently —NR^{20C}NR^{20A}R^{20B}. In embodiments, R²⁰ is independently -ONR^{20A}R^{20B}. In embodiments, R²⁰ is independently —NHC(O)NR^{20C}NR^{20A}R^{20B}. In embodiments, R²⁰ is independently —NHC(O)NR^{20A}R^{20B}. In embodiments, R²⁰ is independently -N(O)ₘ₁₀. In embodiments, R²⁰ is independently —NR^{20A}R^{20B}. In embodiments, R²⁰ is independently —C(O)R^{20C}. In embodiments, R²⁰ is independently —C(O)—OR^{20C}. In embodiments, R²⁰ is independently —C(O)NR^{20A}R^{20B}. In embodiments, R²⁰ is independently -OR^{20D}. In embodiments, R²⁰ is independently —NR^{20A}SO₂R^{20D}. In embodiments, R²⁰ is independently —NR^{20A}C(O)R^{20C}. In embodiments, R²⁰ is independently —NR^{20A}C(O)OR^{20C}. In embodiments, R²⁰ is independently —NR^{20A}OR^{20C}. In embodiments, R²⁰ is independently —SF₅. In embodiments, R²⁰ is independently —N₃.

In embodiments, R²⁰ is independently —SCH₃. In embodiments, R²⁰ is independently —OCH₃. In embodiments, R²⁰ is independently unsubstituted C₁—C₄ alkyl. In embodiments, R²⁰ is independently unsubstituted cyclopropyl. In embodiments, R²⁰ is independently unsubstituted phenyl. In embodiments, R²⁰ is independently hydrogen. In embodiments, R²⁰ is independently —CCl₃. In embodiments, R²⁰ is independently —CBr₃. In embodiments, R²⁰ is independently —CF₃. In embodiments, R²⁰ is independently —CI₃. In embodiments, R²⁰ is independently —CHCl₂. In embodiments, R²⁰ is independently —CHBr₂. In embodiments, R²⁰ is independently —CHF₂. In embodiments, R²⁰ is independently —CHI2. In embodiments, R²⁰ is independently —CH₂Cl. In embodiments, R²⁰ is independently -CH₂Br. In embodiments, R²⁰ is independently —CH₂F. In embodiments, R²⁰ is independently —CH₂I. In embodiments, R²⁰ is independently —CN. In embodiments, R²⁰ is independently —OH. In embodiments, R²⁰ is independently —NH₂. In embodiments, R²⁰ is independently —COOH. In embodiments, R²⁰ is independently —CONH₂. In embodiments, R²⁰ is independently —OCCl₃. In embodiments, R²⁰ is independently —OCF₃. In embodiments, R²⁰ is independently —OCBr₃. In embodiments, R²⁰ is independently —OCI₃. In embodiments, R²⁰ is independently —OCHCl₂. In embodiments, R²⁰ is independently —OCHBr₂. In embodiments, R²⁰ is independently —OCHI2. In embodiments, R²⁰ is independently —OCHF₂. In embodiments, R²⁰ is independently —OCH₂Cl. In embodiments, R²⁰ is independently —OCH2Br. In embodiments, R²⁰ is independently —OCH₂I. In embodiments, R²⁰ is independently —OCH₂F. In embodiments, R²⁰ is independently halogen. In embodiments, R²⁰ is independently —NO₂. In embodiments, R²⁰ is independently —OCH₃. In embodiments, R²⁰ is independently —OCH₂CH₃. In embodiments, R²⁰ is independently —OCH(CH₃)₂. In embodiments, R²⁰ is independently —OC(CH₃)₃. In embodiments, R²⁰ is independently —CH₃. In embodiments, R²⁰ is independently —CH₂CH₃. In embodiments, R²⁰ is independently —CH(CH₃)₂. In embodiments, R²⁰ is independently —C(CH₃)₃. In embodiments, R²⁰ is independently unsubstituted cyclopropyl. In embodiments, R²⁰ is independently unsubstituted cyclobutyl. In embodiments, R²⁰ is independently unsubstituted cyclopentyl. In embodiments, R²⁰ is independently unsubstituted cyclohexyl. In embodiments, R²⁰ is independently substituted or unsubstituted alkyl (e.g., C₁—C₈, C₁—C₆, or C₁—C₄). In embodiments, R²⁰ is independently substituted or unsubstituted heteroalkyl (e.g., 2 to 8 membered, 2 to 6 membered, or 2 to 4 membered). In embodiments, R²⁰ is independently substituted or unsubstituted cycloalkyl (e.g., C₃—C₈, C₃—C₆, or C₅—C₆). In embodiments, R²⁰ is independently substituted or unsubstituted heterocycloalkyl (e.g., 3 to 8 membered, 3 to 6 membered, or 5 to 6 membered). In embodiments, R²⁰ is independently substituted or unsubstituted aryl (e.g., C₆—C₁₀, C₁₀, or phenyl). In embodiments, R²⁰ is independently substituted or unsubstituted heteroaryl (e.g., 5 to 10 membered, 5 to 9 membered, or 5 to 6 membered). In embodiments, R²⁰ is independently unsubstituted alkyl (e.g., C₁—C₈, C₁—C₆, or C₁—C₄). In embodiments, R²⁰ is independently unsubstituted heteroalkyl (e.g., 2 to 8 membered, 2 to 6 membered, or 2 to 4 membered). In embodiments, R²⁰ is independently unsubstituted cycloalkyl (e.g., C₃—C₈, C₃—C₆, or C₅—C₆). In embodiments, R²⁰ is independently unsubstituted heterocycloalkyl (e.g., 3 to 8 membered, 3 to 6 membered, or 5 to 6 membered). In embodiments, R²⁰ is independently unsubstituted aryl (e.g., C₆—C₁₀, C₁₀, or phenyl). In embodiments, R²⁰ is independently unsubstituted heteroaryl (e.g., 5 to 10 membered, 5 to 9 membered, or 5 to 6 membered).

In embodiments, two adjacent R²⁰ substituents are joined to form a substituted or unsubstituted cycloalkyl (e.g., C₃—C₈, C₃—C₆, or C₅—C₆). In embodiments, two adjacent R²⁰ substituents are joined to form a substituted or unsubstituted heterocycloalkyl (e.g., 3 to 8 membered, 3 to 6 membered, or 5 to 6 membered). In embodiments, two adjacent R²⁰ substituents are joined to form a substituted or unsubstituted aryl (e.g., C₆—C₁₀, C₁₀, or phenyl). In embodiments, two adjacent R²⁰ substituents are joined to form a substituted or unsubstituted heteroaryl (e.g., 5 to 10 membered, 5 to 9 membered, or 5 to 6 membered). In embodiments, two adjacent R²⁰ substituents are joined to form an unsubstituted cycloalkyl (e.g., C₃—C₈, C₃—C₆, or C₅—C₆). In embodiments, two adjacent R²⁰ substituents are joined to form an unsubstituted heterocycloalkyl (e.g., 3 to 8 membered, 3 to 6 membered, or 5 to 6 membered). In embodiments, two adjacent R²⁰ substituents are joined to form an unsubstituted aryl (e.g., C₆—C₁₀, C₁₀, or phenyl). In embodiments, two adjacent R²⁰ substituents are joined to form an unsubstituted heteroaryl (e.g., 5 to 10 membered, 5 to 9 membered, or 5 to 6 membered).

R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered),; R^{20A} and R^{20B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

In embodiments, R^{20A} is independently halogen. In embodiments, R^{20A} isindependently —SCH₃. In embodiments, R^{20A} is independently —OCH₃. In embodiments, R^{20A} is independently unsubstituted C₁—C₄ alkyl. In embodiments, R^{20A} is independently unsubstituted cyclopropyl. In embodiments, R^{20A} is independently unsubstituted phenyl. In embodiments, R^{20A} is independently hydrogen. In embodiments, R^{20A} is independently —CCl₃. In embodiments, R^{20A} is independently —CBr₃. In embodiments, R^{20A} is independently —CF₃. In embodiments, R^{20A} is independently —CI₃. In embodiments, R^{20A} is independently —CHCl₂. In embodiments, R^{20A} is independently —CHBr₂. In embodiments, R^{20A} is independently —CHF₂. In embodiments, R^{20A} is independently —CHI2. In embodiments, R^{20A} is independently —CH₂Cl. In embodiments, R^{20A} is independently -CH₂Br. In embodiments, R^{20A} is independently —CH₂F. In embodiments, R^{20A} is independently —CH₂I. In embodiments, R^{20A} is independently —CN. In embodiments, R^{20A} is independently —OH. In embodiments, R^{20A} is independently —NH₂. In embodiments, R^{20A} is independently —COOH. In embodiments, R^{20A} is independently —CONH₂. In embodiments, R^{20A} is independently —OCCl₃. In embodiments, R^{20A} is independently —OCF₃. In embodiments, R^{20A} is independently —OCBr₃. In embodiments, R^{20A} is independently —OCI₃. In embodiments, R^{20A} is independently —OCHCl₂. In embodiments, R^{20A} is independently —OCHBr₂. In embodiments, R^{20A} is independently —OCHI2. In embodiments, R^{20A} is independently —OCHF₂. In embodiments, R^{20A} is independently —OCH₂Cl. In embodiments, R^{20A} is independently —OCH2Br. In embodiments, R^{20A} is independently -OCH₂I. In embodiments, R^{20A} is independently —OCH₂F. In embodiments, R^{20A} is independently halogen. In embodiments, R^{20A} is independently —NO₂. In embodiments, R^{20A} is independently —OCH₃. In embodiments, R^{20A} is independently —OCH₂CH₃. In embodiments, R^{20A} is independently —OCH(CH₃)₂. In embodiments, R^{20A} is independently -OC(CH₃)₃. In embodiments, R^{20A} is independently —CH₃. In embodiments, R^{20A} is independently —CH₂CH₃. In embodiments, R^{20A} is independently —CH(CH₃)₂. In embodiments, R^{20A} is independently —C(CH₃)₃. In embodiments, R^{20A} is independently unsubstituted cyclopropyl. In embodiments, R^{20A} is independently unsubstituted cyclobutyl. In embodiments, R^{20A} is independently unsubstituted cyclopentyl. In embodiments, R^{20A} is independently unsubstituted cyclohexyl. In embodiments, R^{20A} is independently substituted or unsubstituted alkyl (e.g., C₁—C₈, C₁—C₆, or C₁—C₄). In embodiments, R^{20A} is independently substituted or unsubstituted heteroalkyl (e.g., 2 to 8 membered, 2 to 6 membered, or 2 to 4 membered). In embodiments, R^{20A} is independently substituted or unsubstituted cycloalkyl (e.g., C₃—C₈, C₃—C₆, or C₅—C₆). In embodiments, R^{20A} is independently substituted or unsubstituted heterocycloalkyl (e.g., 3 to 8 membered, 3 to 6 membered, or 5 to 6 membered). In embodiments, R^{20A} is independently substituted or unsubstituted aryl (e.g., C₆—C₁₀, C₁₀, or phenyl). In embodiments, R^{20A} is independently substituted or unsubstituted heteroaryl (e.g., 5 to 10 membered, 5 to 9 membered, or 5 to 6 membered). In embodiments, R^{20A} is independently unsubstituted alkyl (e.g., C₁—C₈, C₁—C₆, or C₁—C₄). In embodiments, R^{20A} is independently unsubstituted heteroalkyl (e.g., 2 to 8 membered, 2 to 6 membered, or 2 to 4 membered). In embodiments, R^{20A} is independently unsubstituted cycloalkyl (e.g., C₃-C₈, C₃—C₆, or C₅—C₆). In embodiments, R^{20A} is independently unsubstituted heterocycloalkyl (e.g., 3 to 8 membered, 3 to 6 membered, or 5 to 6 membered). In embodiments, R^{20A} is independently unsubstituted aryl (e.g., C₆—C₁₀, C₁₀, or phenyl). In embodiments, R^{20A} is independently unsubstituted heteroaryl (e.g., 5 to 10 membered, 5 to 9 membered, or 5 to 6 membered).

In embodiments, R^{20B} is independently halogen. In embodiments, R^{20B} is independently —SCH₃. In embodiments, R^{20B} is independently —OCH₃. In embodiments, R^{20B} is independently unsubstituted C₁—C₄ alkyl. In embodiments, R^{20B} is independently unsubstituted cyclopropyl. In embodiments, R^{20B} is independently unsubstituted phenyl. In embodiments, R^{20B} is independently hydrogen. In embodiments, R^{20B} is independently —CCl₃. In embodiments, R^{20B} is independently —CBr₃. In embodiments, R^{20B} is independently —CF₃. In embodiments, R^{20B} is independently —CI₃. In embodiments, R^{20B} is independently —CHCl₂. In embodiments, R^{20B} is independently —CHBr₂. In embodiments, R^{20B} is independently —CHF₂. In embodiments, R^{20B} is independently —CHI2. In embodiments, R^{20B} is independently —CH₂Cl. In embodiments, R^{20B} is independently —CH2Br. In embodiments, R^{20B} is independently —CH₂F. In embodiments, R^{20B} is independently —CH₂I. In embodiments, R^{20B} is independently —CN. In embodiments, R^{20B} is independently —OH. In embodiments, R^{20B} is independently —NH₂. In embodiments, R^{20B} is independently —COOH. In embodiments, R^{20B} is independently —CONH₂. In embodiments, R^{20B} is independently —OCCl₃. In embodiments, R^{20B} is independently —OCF₃. In embodiments, R^{20B} is independently —OCBr₃. In embodiments, R^{20B} is independently —OCI₃. In embodiments, R^{20B} is independently —OCHCl₂. In embodiments, R^{20B} is independently —OCHBr₂. In embodiments, R^{20B} is independently —OCHI2. In embodiments, R^{20B} is independently —OCHF₂. In embodiments, R^{20B} is independently —OCH₂Cl. In embodiments, R^{20B} is independently —OCH2Br. In embodiments, R^{20B} is independently -OCH₂I. In embodiments, R^{20B} is independently —OCH₂F. In embodiments, R^{20B} is independently halogen. In embodiments, R^{20B} is independently —NO₂. In embodiments, R^{20B} is independently —OCH₃. In embodiments, R^{20B} is independently —OCH₂CH₃. In embodiments, R^{20B} is independently —OCH(CH₃)₂. In embodiments, R^{20B} is independently —OC(CH₃)₃. In embodiments, R^{20B} is independently —CH₃. In embodiments, R^{20B} is independently —CH₂CH₃. In embodiments, R^{20B} is independently —CH(CH₃)₂. In embodiments, R^{20B} is independently —C(CH₃)₃. In embodiments, R^{20B} is independently unsubstituted cyclopropyl. In embodiments, R^{20B} is independently unsubstituted cyclobutyl. In embodiments, R^{20B} is independently unsubstituted cyclopentyl. In embodiments, R^{20B} is independently unsubstituted cyclohexyl. In embodiments, R^{20B} is independently substituted or unsubstituted alkyl (e.g., C₁—C₈, C₁—C₆, or C₁—C₄). In embodiments, R^{20B} is independently substituted or unsubstituted heteroalkyl (e.g., 2 to 8 membered, 2 to 6 membered, or 2 to 4 membered). In embodiments, R^{20B} is independently substituted or unsubstituted cycloalkyl (e.g., C₃—C₈, C₃—C₆, or C₅—C₆). In embodiments, R^{20B} is independently substituted or unsubstituted heterocycloalkyl (e.g., 3 to 8 membered, 3 to 6 membered, or 5 to 6 membered). In embodiments, R^{20B} is independently substituted or unsubstituted aryl (e.g., C₆—C₁₀, C₁₀, or phenyl). In embodiments, R^{20B} is independently substituted or unsubstituted heteroaryl (e.g., 5 to 10 membered, 5 to 9 membered, or 5 to 6 membered). In embodiments, R^{20B} is independently unsubstituted alkyl (e.g., C₁—C₈, C₁—C₆, or C₁—C₄). In embodiments, R^{20B} is independently unsubstituted heteroalkyl (e.g., 2 to 8 membered, 2 to 6 membered, or 2 to 4 membered). In embodiments, R^{20B} is independently unsubstituted cycloalkyl (e.g., C₃-C₈, C₃—C₆, or C₅—C₆). In embodiments, R^{20B} is independently unsubstituted heterocycloalkyl (e.g., 3 to 8 membered, 3 to 6 membered, or 5 to 6 membered). In embodiments, R^{20B} is independently unsubstituted aryl (e.g., C₆—C₁₀, C₁₀, or phenyl). In embodiments, R^{20B} is independently unsubstituted heteroaryl (e.g., 5 to 10 membered, 5 to 9 membered, or 5 to 6 membered).

In embodiments, R^{20A} and R^{20B} substituents bonded to the same nitrogen atom are joined to form a substituted or unsubstituted heterocycloalkyl (e.g., 3 to 8 membered, 3 to 6 membered, or 5 to 6 membered). In embodiments, R^{20A} and R^{20B} substituents bonded to the same nitrogen atom are joined to form a substituted or unsubstituted heteroaryl (e.g., 5 to 10 membered, 5 to 9 membered, or 5 to 6 membered). In embodiments, R^{20A} and R^{20B} substituents bonded to the same nitrogen atom are joined to form an unsubstituted heterocycloalkyl (e.g., 3 to 8 membered, 3 to 6 membered, or 5 to 6 membered). In embodiments, R^{20A} and R^{20B} substituents bonded to the same nitrogen atom are joined to form an unsubstituted heteroaryl (e.g., 5 to 10 membered, 5 to 9 membered, or 5 to 6 membered).

In embodiments, R^{20C} is independently halogen. In embodiments, R^{20C} is independently —SCH₃. In embodiments, R^{20C} is independently —OCH₃. In embodiments, R^{20C} is independently unsubstituted C₁—C₄ alkyl. In embodiments, R^{20C} is independently unsubstituted cyclopropyl. In embodiments, R^{20C} is independently unsubstituted phenyl. In embodiments, R^{20C} is independently hydrogen. In embodiments, R^{20C} is independently —CCl₃. In embodiments, R^{20C} is independently —CBr₃. In embodiments, R^{20C} is independently —CF₃. In embodiments, R^{20C} is independently —CI₃. In embodiments, R^{20C} is independently —CHCl₂. In embodiments, R^{20C} is independently —CHBr₂. In embodiments, R^{20C} is independently —CHF₂. In embodiments, R^{20C} is independently —CHI2. In embodiments, R^{20C} is independently —CH₂Cl. In embodiments, R^{20C} is independently —CH2Br. In embodiments, R^{20C} is independently —CH₂F. In embodiments, R^{20C} is independently —CH₂I. In embodiments, R^{20C} is independently —CN. In embodiments, R^{20C} is independently —OH. In embodiments, R^{20C} is independently —NH₂. In embodiments, R^{20C} is independently —COOH. In embodiments, R^{20C} is independently —CONH₂. In embodiments, R^{20C} is independently —OCCl₃. In embodiments, R^{20C} is independently —OCF₃. In embodiments, R^{20C} is independently —OCBr₃. In embodiments, R^{20C} is independently —OCI₃. In embodiments, R^{20C} is independently —OCHCl₂. In embodiments, R^{20C} is independently —OCHBr₂. In embodiments, R^{20C} is independently —OCHI2. In embodiments, R^{20C} is independently —OCHF₂. In embodiments, R^{20C} is independently —OCH₂Cl. In embodiments, R^{20C} is independently —OCH₂Br. In embodiments, R^{20C} is independently —OCH₂I. In embodiments, R^{20C} is independently —OCH₂F. In embodiments, R^{20C} is independently halogen. In embodiments, R^{20C} is independently —NO₂. In embodiments, R^{20C} is independently —OCH₃. In embodiments, R^{20C} is independently —OCH₂CH₃. In embodiments, R^{20C} is independently —OCH(CH₃)₂. In embodiments, R^{20C} is independently —OC(CH₃)₃. In embodiments, R^{20C} is independently —CH₃. In embodiments, R^{20C} is independently —CH₂CH₃. In embodiments, R^{20C} is independently —CH(CH₃)₂. In embodiments, R^{20C} is independently -C(CH₃)₃. In embodiments, R^{20C} is independently unsubstituted cyclopropyl. In embodiments, R^{20C} is independently unsubstituted cyclobutyl. In embodiments, R^{20C} is independently unsubstituted cyclopentyl. In embodiments, R^{20C} is independently unsubstituted cyclohexyl. In embodiments, R^{20C} is independently substituted or unsubstituted alkyl (e.g., C₁—C₈, C₁—C₆, or C₁—C₄). In embodiments, R^{20C} is independently substituted or unsubstituted heteroalkyl (e.g., 2 to 8 membered, 2 to 6 membered, or 2 to 4 membered). In embodiments, R^{20C} is independently substituted or unsubstituted cycloalkyl (e.g., C₃—C₈, C₃-C₆, or C₅—C₆). In embodiments, R^{20C} is independently substituted or unsubstituted heterocycloalkyl (e.g., 3 to 8 membered, 3 to 6 membered, or 5 to 6 membered). In embodiments, R^{20C} is independently substituted or unsubstituted aryl (e.g., C₆—C₁₀, C₁₀, or phenyl). In embodiments, R^{20C} is independently substituted or unsubstituted heteroaryl (e.g., 5 to 10 membered, 5 to 9 membered, or 5 to 6 membered). In embodiments, R^{20C} is independently unsubstituted alkyl (e.g., C₁—C₈, C₁—C₆, or C₁—C₄). In embodiments, R^{20C} is independently unsubstituted heteroalkyl (e.g., 2 to 8 membered, 2 to 6 membered, or 2 to 4 membered). In embodiments, R^{20C} is independently unsubstituted cycloalkyl (e.g., C₃—C₈, C₃-C₆, or C₅—C₆). In embodiments, R^{20C} is independently unsubstituted heterocycloalkyl (e.g., 3 to 8 membered, 3 to 6 membered, or 5 to 6 membered). In embodiments, R^{20C} is independently unsubstituted aryl (e.g., C₆—C₁₀, C₁₀, or phenyl). In embodiments, R^{20C} is independently unsubstituted heteroaryl (e.g., 5 to 10 membered, 5 to 9 membered, or 5 to 6 membered).

In embodiments, R^{20D} is independently halogen. In embodiments, R^{20D} is independently —SCH₃. In embodiments, R^{20D} is independently —OCH₃. In embodiments, R^{20D} is independently unsubstituted C₁—C₄ alkyl. In embodiments, R^{20D} is independently unsubstituted cyclopropyl. In embodiments, R^{20D} is independently unsubstituted phenyl. In embodiments, R^{20D} is independently hydrogen. In embodiments, R^{20D} is independently —CCl₃. In embodiments, R^{20D} is independently —CBr₃. In embodiments, R^{20D} is independently —CF₃. In embodiments, R^{20D} is independently —CI₃. In embodiments, R^{20D} is independently —CHCl₂. In embodiments, R^{20D} is independently —CHBr₂. In embodiments, R^{20D} is independently —CHF₂. In embodiments, R^{20D} is independently —CHI2. In embodiments, R^{20D} is independently —CH₂Cl. In embodiments, R^{20D} is independently -CH₂Br. In embodiments, R^{20D} is independently —CH₂F. In embodiments, R^{20D} is independently —CH₂I. In embodiments, R^{20D} is independently —CN. In embodiments, R^{20D} is independently —OH. In embodiments, R^{20D} is independently —NH₂. In embodiments, R^{20D} is independently —COOH. In embodiments, R^{20D} is independently —CONH₂. In embodiments, R^{20D} is independently —OCCl₃. In embodiments, R^{20D} is independently —OCF₃. In embodiments, R^{20D} is independently —OCBr₃. In embodiments, R^{20D} is independently —OCI₃. In embodiments, R^{20D} is independently —OCHCl₂. In embodiments, R^{20D} is independently —OCHBr₂. In embodiments, R^{20D} is independently —OCHI2. In embodiments, R^{20D} is independently —OCHF₂. In embodiments, R^{20D} is independently —OCH₂Cl. In embodiments, R^{20D} is independently —OCH₂Br. In embodiments, R^{20D} is independently —OCH₂I. In embodiments, R^{20D} is independently —OCH₂F. In embodiments, R^{20D} is independently halogen. In embodiments, R^{20D} is independently —NO₂. In embodiments, R^{20D} is independently —OCH₃. In embodiments, R^{20D} is independently -OCH₂CH₃. In embodiments, R^{20D} is independently —OCH(CH₃)₂. In embodiments, R^{20D} is independently-OC(CH₃)₃. In embodiments, R^{20D} is independently —CH₃. In embodiments, R^{20D} is independently —CH₂CH₃. In embodiments, R^{20D} is independently —CH(CH₃)₂. In embodiments, R^{20D} is independently —C(CH₃)₃. In embodiments, R^{20D} is independently unsubstituted cyclopropyl. In embodiments, R^{20D} is independently unsubstituted cyclobutyl. In embodiments, R^{20D} is independently unsubstituted cyclopentyl. In embodiments, R^{20D} is independently unsubstituted cyclohexyl. In embodiments, R^{20D} is independently substituted or unsubstituted alkyl (e.g., C₁—C₈, C₁—C₆, or C₁—C₄). In embodiments, R^{20D} is independently substituted or unsubstituted heteroalkyl (e.g., 2 to 8 membered, 2 to 6 membered, or 2 to 4 membered). In embodiments, R^{20D} is independently substituted or unsubstituted cycloalkyl (e.g., C₃—C₈, C₃—C₆, or C₅—C₆). In embodiments, R^{20D} is independently substituted or unsubstituted heterocycloalkyl (e.g., 3 to 8 membered, 3 to 6 membered, or 5 to 6 membered). In embodiments, R^{20D} is independently substituted or unsubstituted aryl (e.g., C₆—C₁₀, C₁₀, or phenyl). In embodiments, R^{20D} is independently substituted or unsubstituted heteroaryl (e.g., 5 to 10 membered, 5 to 9 membered, or 5 to 6 membered). In embodiments, R^{20D} is independently unsubstituted alkyl (e.g., C₁—C₈, C₁—C₆, or C₁—C₄). In embodiments, R^{20D} is independently unsubstituted heteroalkyl (e.g., 2 to 8 membered, 2 to 6 membered, or 2 to 4 membered). In embodiments, R^{20D} is independently unsubstituted cycloalkyl (e.g., C₃-C₈, C₃—C₆, or C₅—C₆). In embodiments, R^{20D} is independently unsubstituted heterocycloalkyl (e.g., 3 to 8 membered, 3 to 6 membered, or 5 to 6 membered). In embodiments, R^{20D} is independently unsubstituted aryl (e.g., C₆—C₁₀, C₁₀, or phenyl). In embodiments, R^{20D} is independently unsubstituted heteroaryl (e.g., 5 to 10 membered, 5 to 9 membered, or 5 to 6 membered). R^{20D} is independently hydrogen or unsubstituted C₁—C₄ alkyl.

In embodiments, R³ is independently halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —NO₂, —SH, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, —CH₃, —CH₂CH₃, —OCH₃, or —OCH₂CH₃. In embodiments, R³ is independently halogen, oxo, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —NO₂, —SH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CH₃, —CH₂CH₃, —OCH₃, or —OCH₂CH₃. In embodiments, R³ is independently —OH, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, —CH₃, —CH₂CH₃, —OCH₃, or —OCH₂CH₃. In embodiments, R³ is independently —OCH₃. In embodiments, R³ is independently halogen, —CCl₃, —CBr₃, —CF₃, —CI₃, —CHCl₂, —CHBr₂, —CHF₂, —CHI2, —CH₂Cl, —CH₂Br, —CH₂F, or —CH₂I. In embodiments, R³ is independently —F or —CF₃. In embodiments, R³ is independently —CF₃. In embodiments, R³ is independently halogen. In embodiments, R³ is independently —CCl₃. In embodiments, R³ is independently —CBr₃. In embodiments, R³ is independently —CF₃. In embodiments, R³ is independently —CI₃. In embodiments, R³ is independently —CHCl₂. In embodiments, R³ is independently —CHBr₂. In embodiments, R³ is independently —CHF₂. In embodiments, R³ is independently —CHI2. In embodiments, R³ is independently —CH₂Cl. In embodiments, R³ is independently —CH2Br. In embodiments, R³ is independently —CH₂F. In embodiments, R³ is independently —CH₂I. In embodiments, R³ is independently —CN. In embodiments, R³ is independently —OH. In embodiments, R³ is independently —NH₂. In embodiments, R³ is independently -COOH. In embodiments, R³ is independently —CONH₂. In embodiments, R³ is independently —NO₂. In embodiments, R³ is independently —SH. In embodiments, R³ is independently —SO₃H. In embodiments, R³ is independently —SO₄H. In embodiments, R³ is independently —SO₂NH₂. In embodiments, R³ is independently —NHNH₂. In embodiments, R³ is independently —ONH₂. In embodiments, R³ is independently —NHC(O)NHNH₂. In embodiments, R³ is independently —NHC(O)NH₂. In embodiments, R³ is independently —NHSO₂H. In embodiments, R³ is independently —NHC(O)H. In embodiments, R³ is independently —NHC(O)OH. In embodiments, R³ is independently —NHOH. In embodiments, R³ is independently —OCCl₃. In embodiments, R³ is independently —OCF₃. In embodiments, R³ is independently —OCBr₃. In embodiments, R³ is independently —OCI₃. In embodiments, R³ is independently —OCHCl₂. In embodiments, R³ is independently —OCHBr₂. In embodiments, R³ is independently —OCHI2. In embodiments, R³ is independently —OCHF₂. In embodiments, R³ is independently —OCH₂Cl. In embodiments, R³ is independently —OCH₂Br. In embodiments, R³ is independently —OCH₂I. In embodiments, R³ is independently —OCH₂F. In embodiments, R³ is independently —SF₅. In embodiments, R³ is independently —N₃. In embodiments, R³ is independently —F. In embodiments, R³ is independently —Cl. In embodiments, R³ is independently —Br. In embodiments, R³ is independently —I. In embodiments, R³ is independently —CH₂OCH₃. In embodiments, R³ is independently —SCH₃. In embodiments, R³ is independently —OCH₃. In embodiments, R³ is independently —CH₂CH₂OCH₃. In embodiments, R³ is independently —SCH₂CH₃. In embodiments, R³ is independently —OCH₂CH₃. In embodiments, R³ is independently —CH₂OCH₂CH₃. In embodiments, R³ is independently unsubstituted C₁—C₄ alkyl. In embodiments, R³ is independently unsubstituted cyclopropyl. In embodiments, R³ is independently hydrogen. In embodiments, R³ is independently —OCH₃. In embodiments, R³ is independently —OCH₂CH₃. In embodiments, R³ is independently —OCH(CH₃)₂. In embodiments, R³ is independently —OC(CH₃)₃. In embodiments, R³ is independently —CH₃. In embodiments, R³ is independently —CH₂CH₃. In embodiments, R³ is independently —CH(CH₃)₂. In embodiments, R³ is independently -C(CH₃)₃. In embodiments, R³ is independently unsubstituted cyclopropyl. In embodiments, R³ is independently unsubstituted cyclobutyl. In embodiments, R³ is independently unsubstituted cyclopentyl. In embodiments, R³ is independently unsubstituted cyclohexyl. In embodiments, R³ is independently substituted or unsubstituted alkyl (e.g., C₁—C₈, C₁—C₆, or C₁—C₄). In embodiments, R³ is independently substituted or unsubstituted heteroalkyl (e.g., 2 to 8 membered, 2 to 6 membered, or 2 to 4 membered). In embodiments, R³ is independently substituted or unsubstituted cycloalkyl (e.g., C₃—C₈, C₃—C₆, or C₅—C₆). In embodiments, R³ is independently substituted or unsubstituted heterocycloalkyl (e.g., 3 to 8 membered, 3 to 6 membered, or 5 to 6 membered). In embodiments, R³ is independently substituted or unsubstituted aryl (e.g., C₆—C₁₀, C₁₀, or phenyl). In embodiments, R³ is independently substituted or unsubstituted heteroaryl (e.g., 5 to 10 membered, 5 to 9 membered, or 5 to 6 membered). In embodiments, R³ is independently unsubstituted alkyl (e.g., C₁—C₈, C₁—C₆, or C₁—C₄). In embodiments, R³ is independently unsubstituted heteroalkyl (e.g., 2 to 8 membered, 2 to 6 membered, or 2 to 4 membered). In embodiments, R³ is independently unsubstituted cycloalkyl (e.g., C₃—C₈, C₃—C₆, or C₅—C₆). In embodiments, R³ is independently unsubstituted heterocycloalkyl (e.g., 3 to 8 membered, 3 to 6 membered, or 5 to 6 membered). In embodiments, R³ is independently unsubstituted aryl (e.g., C₆—C₁₀, C₁₀, or phenyl). In embodiments, R³ is independently unsubstituted heteroaryl (e.g., 5 to 10 membered, 5 to 9 membered, or 5 to 6 membered).

In embodiments, R³ is independently substituted or unsubstituted 3 to 8 membered heterocycloalkyl. In embodiments, R³ is independently unsubstituted 3 to 8 membered heterocycloalkyl. In embodiments, R³ is independently substituted or unsubstituted piperazinyl. In embodiments, R³ is independently unsubstituted piperazinyl. In embodiments, R³ is independently substituted piperazinyl. In embodiments, R³ is independently piperazinyl substituted with unsubstituted C₁—C₄ alkyl. In embodiments, R³ is independently piperazinyl substituted with substituted or unsubstituted 2 to 8 membered heteroalkyl. In embodiments, R³ is independently

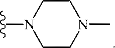

In embodiments, R³ is independently

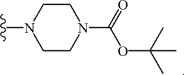

In embodiments, two adjacent R³ substituents are joined to form a substituted or unsubstituted cycloalkyl (e.g., C₃—C₈, C₃—C₆, or C₅—C₆). In embodiments, two adjacent R³ substituents are joined to form a substituted or unsubstituted heterocycloalkyl (e.g., 3 to 8 membered, 3 to 6 membered, or 5 to 6 membered). In embodiments, two adjacent R³ substituents are joined to form a substituted or unsubstituted aryl (e.g., C₆—C₁₀, C₁₀, or phenyl). In embodiments, two adjacent R³ substituents are joined to form a substituted or unsubstituted heteroaryl (e.g., 5 to 10 membered, 5 to 9 membered, or 5 to 6 membered). In embodiments, two adjacent R³ substituents are joined to form an unsubstituted cycloalkyl (e.g., C₃—C₈, C₃—C₆, or C₅—C₆). In embodiments, two adjacent R³ substituents are joined to form an unsubstituted heterocycloalkyl (e.g., 3 to 8 membered, 3 to 6 membered, or 5 to 6 membered). In embodiments, two adjacent R³ substituents are joined to form an unsubstituted aryl (e.g., C₆—C₁₀, C₁₀, or phenyl). In embodiments, two adjacent R³ substituents are joined to form an unsubstituted heteroaryl (e.g., 5 to 10 membered, 5 to 9 membered, or 5 to 6 membered).

In embodiments, z3 is independently 0. In embodiments, z3 is independently 1. In embodiments, z3 is independently 2. In embodiments, z3 is independently 3. In embodiments, z3 is independently 4.

In embodiments, R³ is independently hydrogen. In embodiments, R³ is independently halogen. In embodiments, R³ is independently —CX³₃. In embodiments, R³ is independently —CHX³₂. In embodiments, R³ is independently —CH₂X³. In embodiments, R³ is independently —OCX³₃. In embodiments, R³ is independently —OCH₂X³. In embodiments, R³ is independently —OCHX³₂. In embodiments, R³ is independently —CN. In embodiments, R³ is independently —SF₅. In embodiments, R³ is independently —N₃. In embodiments, R³ is independently —SOₙ₃R^{3D}. In embodiments, R³ is independently —SOᵥ₃NR^{3A}R^{3B}. In embodiments, R³ is independently —NR^{3C}NR^{3A}R^{3B}. In embodiments, R³ is independently —ONR^{3A}R^{3B}. In embodiments, R³ is independently —NHC(O)NR^{3C}NR^{3A}R^{3B}. In embodiments, R³ is independently —NHC(O)NR^{3A}R^{3B}. In embodiments, R³ is independently —N(O)ₘ₃. In embodiments, R³ is independently —NR^{3A}R^{3B}. In embodiments, R³ is independently —C(O)R^{3C}. In embodiments, R³ is independently —C(O)—OR^{3C}. In embodiments, R³ is independently —C(O)NR^{3A}R^{3B}. In embodiments, R³ is independently —OR^{3D}. In embodiments, R³ is independently —NR³⁴SO₂R^{3D}. In embodiments, R³ is independently —NR^{3A}C(O)R^{3C}. In embodiments, R³ is independently —NR^{3A}C(O)OR^{3C}. In embodiments, R³ is independently —NR^{3A}OR^{3C}.

In embodiments, X³ is independently —F. In embodiments, X³ is independently —Cl. In embodiments, X³ is independently —Br. In embodiments, X³ is independently —I.

In embodiments, n3 is independently 0. In embodiments, n3 is independently 1. In embodiments, n3 is independently 2. In embodiments, n3 is independently 3. In embodiments, n3 is independently 4.

In embodiments, m3 is independently 1. In embodiments, m3 is independently 2. In embodiments, v3 is independently 1. In embodiments, v3 is independently 2.

In embodiments, R^{3A} is independently hydrogen. In embodiments, R^{3A} is independently —CCl₃. In embodiments, R^{3A} is independently —CBr₃. In embodiments, R^{3A} is independently —CF₃. In embodiments, R^{3A} is independently —CI₃. In embodiments, R^{3A} is independently —CHCl₂. In embodiments, R^{3A} is independently —CHBr₂. In embodiments, R^{3A} is independently —CHF₂. In embodiments, R^{3A} is independently —CHI2. In embodiments, R^{3A} is independently —CH₂Cl. In embodiments, R^{3A} is independently —CH2Br. In embodiments, R^{3A} is independently —CH₂F. In embodiments, R^{3A} is independently —CH₂I. In embodiments, R^{3A} is independently —CN. In embodiments, R^{3A} is independently —OH. In embodiments, R^{3A} is independently —NH₂. In embodiments, R^{3A} is independently -COOH. In embodiments, R^{3A} is independently —CONH₂. In embodiments, R^{3A} is independently —OCCl₃. In embodiments, R^{3A} is independently —OCF₃. In embodiments, R^{3A} is independently —OCBr₃. In embodiments, R^{3A} is independently —OCI₃. In embodiments, R^{3A} is independently —OCHCl₂. In embodiments, R^{3A} is independently —OCHBr₂. In embodiments, R^{3A} is independently —OCHI2. In embodiments, R^{3A} is independently —OCHF₂. In embodiments, R^{3A} is independently —OCH₂Cl. In embodiments, R^{3A} is independently —OCH₂Br. In embodiments, R^{3A} is independently —OCH₂I. In embodiments, R^{3A} is independently —OCH₂F. In embodiments, R^{3A} is independently halogen. In embodiments, R^{3A} is independently —NO₂. In embodiments, R^{3A} is independently —OCH₃. In embodiments, R^{3A} is independently -OCH₂CH₃. In embodiments, R^{3A} is independently —OCH(CH₃)₂. In embodiments, R^{3A} is independently —OC(CH₃)₃. In embodiments, R^{3A} is independently —CH₃. In embodiments, R^{3A} is independently —CH₂CH₃. In embodiments, R^{3A} is independently —CH(CH₃)₂. In embodiments, R^{3A} is independently —C(CH₃)₃. In embodiments, R^{3A} is independently unsubstituted cyclopropyl. In embodiments, R^{3A} is independently unsubstituted cyclobutyl. In embodiments, R^{3A} is independently unsubstituted cyclopentyl. In embodiments, R^{3A} is independently unsubstituted cyclohexyl. In embodiments, R^{3A} is independently substituted or unsubstituted alkyl (e.g., C₁—C₈, C₁—C₆, or C₁—C₄). In embodiments, R^{3A} is independently substituted or unsubstituted heteroalkyl (e.g., 2 to 8 membered, 2 to 6 membered, or 2 to 4 membered). In embodiments, R^{3A} is independently substituted or unsubstituted cycloalkyl (e.g., C₃—C₈, C₃—C₆, or C₅—C₆). In embodiments, R^{3A} is independently substituted or unsubstituted heterocycloalkyl (e.g., 3 to 8 membered, 3 to 6 membered, or 5 to 6 membered). In embodiments, R^{3A} is independently substituted or unsubstituted aryl (e.g., C₆-C₁₀, C₁₀, or phenyl). In embodiments, R^{3A} is independently substituted or unsubstituted heteroaryl (e.g., 5 to 10 membered, 5 to 9 membered, or 5 to 6 membered).

In embodiments, R^{3B} is independently hydrogen. In embodiments, R^{3B} is independently —CCl₃. In embodiments, R^{3B} is independently —CBr₃. In embodiments, R^{3B} is independently —CF₃. In embodiments, R^{3B} is independently —CI₃. In embodiments, R^{3B} is independently —CHCl₂. In embodiments, R^{3B} is independently —CHBr2. In embodiments, R^{3B} is independently —CHF₂. In embodiments, R^{3B} is independently —CHI2. In embodiments, R^{3B} is independently —CH₂Cl. In embodiments, R^{3B} is independently —CH2Br. In embodiments, R^{3B} is independently —CH₂F. In embodiments, R^{3B} is independently —CH₂I. In embodiments, R^{3B} is independently —CN. In embodiments, R^{3B} is independently —OH. In embodiments, R^{3B} is independently —NH₂. In embodiments, R^{3B} is independently -COOH. In embodiments, R^{3B} is independently —CONH₂. In embodiments, R^{3B} is independently —OCCl₃. In embodiments, R^{3B} is independently —OCF₃. In embodiments, R^{3B} is independently —OCBr₃. In embodiments, R^{3B} is independently —OCI₃. In embodiments, R^{3B} is independently —OCHCl₂. In embodiments, R^{3B} is independently —OCHBr₂. In embodiments, R^{3B} is independently —OCHI2. In embodiments, R^{3B} is independently —OCHF₂. In embodiments, R^{3B} is independently —OCH₂Cl. In embodiments, R^{3B} is independently —OCH₂Br. In embodiments, R^{3B} is independently —OCH₂I. In embodiments, R^{3B} is independently —OCH₂F. In embodiments, R^{3B} is independently halogen. In embodiments, R^{3B} is independently —NO₂. In embodiments, R^{3B} is independently —OCH₃. In embodiments, R^{3B} is independently -OCH₂CH₃. In embodiments, R^{3B} is independently —OCH(CH₃)₂. In embodiments, R^{3B} is independently-OC(CH₃)₃. In embodiments, R^{3B} is independently —CH₃. In embodiments, R^{3B} is independently —CH₂CH₃. In embodiments, R^{3B} is independently —CH(CH₃)₂. In embodiments, R^{3B} is independently —C(CH₃)₃. In embodiments, R^{3B} is independently unsubstituted cyclopropyl. In embodiments, R^{3B} is independently unsubstituted cyclobutyl. In embodiments, R^{3B} is independently unsubstituted cyclopentyl. In embodiments, R^{3B} is independently unsubstituted cyclohexyl. In embodiments, R^{3B} is independently substituted or unsubstituted alkyl (e.g., C₁—C₈, C₁—C₆, or C₁—C₄). In embodiments, R^{3B} is independently substituted or unsubstituted heteroalkyl (e.g., 2 to 8 membered, 2 to 6 membered, or 2 to 4 membered). In embodiments, R^{3B} is independently substituted or unsubstituted cycloalkyl (e.g., C₃—C₈, C₃—C₆, or C₅—C₆). In embodiments, R^{3B} is independently substituted or unsubstituted heterocycloalkyl (e.g., 3 to 8 membered, 3 to 6 membered, or 5 to 6 membered). In embodiments, R^{3B} is independently substituted or unsubstituted aryl (e.g., C₆-C₁₀, C₁₀, or phenyl). In embodiments, R^{3B} is independently substituted or unsubstituted heteroaryl (e.g., 5 to 10 membered, 5 to 9 membered, or 5 to 6 membered).

In embodiments, R^{3A} and R^{3B} substituents bonded to the same nitrogen atom are joined to form a substituted or unsubstituted heterocycloalkyl (e.g., 3 to 8 membered, 3 to 6 membered, or 5 to 6 membered). In embodiments, R^{3A} and R^{3B} substituents bonded to the same nitrogen atom are joined to form a substituted or unsubstituted 3 to 6 membered heterocycloalkyl. In embodiments, R^{3A} and R^{3B} bonded to the same nitrogen atom are joined to form a substituted or unsubstituted piperazinyl. In embodiments, R^{3A} and R^{3B} substituents bonded to the same nitrogen atom are joined to form a substituted or unsubstituted heteroaryl (e.g., 5 to 10 membered, 5 to 9 membered, or 5 to 6 membered). In embodiments, R^{3A} and R^{3B} substituents bonded to the same nitrogen atom are joined to form a substituted or unsubstituted 5 to 6 membered heteroaryl.

In embodiments, R^{3C} is independently hydrogen. In embodiments, R^{3C} is independently —CCl₃. In embodiments, R^{3C} is independently —CBr₃. In embodiments, R^{3C} is independently —CF₃. In embodiments, R^{3C} is independently —CI₃. In embodiments, R^{3C} is independently —CHCl₂. In embodiments, R^{3C} is independently —CHBr₂. In embodiments, R^{3C} is independently —CHF₂. In embodiments, R^{3C} is independently —CHI2. In embodiments, R^{3C} is independently —CH₂Cl. In embodiments, R^{3C} is independently —CH₂Br. In embodiments, R^{3C} is independently —CH₂F. In embodiments, R^{3C} is independently —CH₂I. In embodiments, R^{3C} is independently —CN. In embodiments, R^{3C} is independently —OH. In embodiments, R^{3C} is independently —NH₂. In embodiments, R^{3C} is independently —COOH. In embodiments, R^{3C} is independently —CONH₂. In embodiments, R^{3C} is independently —OCCl₃. In embodiments, R^{3C} is independently —OCF₃. In embodiments, R^{3C} is independently —OCBr₃. In embodiments, R^{3C} is independently —OCI₃. In embodiments, R^{3C} is independently —OCHCl₂. In embodiments, R^{3C} is independently —OCHBr₂. In embodiments, R^{3C} is independently —OCHI2. In embodiments, R^{3C} is independently —OCHF₂. In embodiments, R^{3C} is independently —OCH₂Cl. In embodiments, R^{3C} is independently —OCH2Br. In embodiments, R^{3C} is independently —OCH₂I. In embodiments, R^{3C} is independently —OCH₂F. In embodiments, R^{3C} is independently halogen. In embodiments, R^{3C} is independently —NO₂. In embodiments, R^{3C} is independently —OCH₃. In embodiments, R^{3C} is independently —OCH₂CH₃. In embodiments, R^{3C} is independently —OCH(CH₃)₂. In embodiments, R^{3C} is independently —OC(CH₃)₃. In embodiments, R^{3C} is independently —CH₃. In embodiments, R^{3C} is independently —CH₂CH₃. In embodiments, R^{3C} is independently —CH(CH₃)₂. In embodiments, R^{3C} is independently —C(CH₃)₃. In embodiments, R^{3C} is independently unsubstituted cyclopropyl. In embodiments, R^{3C} is independently unsubstituted cyclobutyl. In embodiments, R^{3C} is independently unsubstituted cyclopentyl. In embodiments, R^{3C} is independently unsubstituted cyclohexyl. In embodiments, R^{3C} is independently substituted or unsubstituted alkyl (e.g., C₁—C₈, C₁—C₆, or C₁—C₄). In embodiments, R^{3C} is independently substituted or unsubstituted heteroalkyl (e.g., 2 to 8 membered, 2 to 6 membered, or 2 to 4 membered). In embodiments, R^{3C} is independently substituted or unsubstituted cycloalkyl (e.g., C₃—C₈, C₃—C₆, or C₅—C₆). In embodiments, R^{3C} is independently substituted or unsubstituted heterocycloalkyl (e.g., 3 to 8 membered, 3 to 6 membered, or 5 to 6 membered). In embodiments, R^{3C} is independently substituted or unsubstituted aryl (e.g., C₆-C₁₀, C₁₀, or phenyl). In embodiments, R^{3C} is independently substituted or unsubstituted heteroaryl (e.g., 5 to 10 membered, 5 to 9 membered, or 5 to 6 membered).

In embodiments, R^{3D} is independently hydrogen. In embodiments, R^{3D} is independently —CCl₃. In embodiments, R^{3D} is independently —CBr₃. In embodiments, R^{3D} is independently —CF₃. In embodiments, R^{3D} is independently —CI₃. In embodiments, R^{3D} is independently —CHCl₂. In embodiments, R^{3D} is independently —CHBr₂. In embodiments, R^{3D} is independently —CHF₂. In embodiments, R^{3D} is independently —CHI2. In embodiments, R^{3D} is independently —CH₂Cl. In embodiments, R^{3D} is independently —CH₂Br. In embodiments, R^{3D} is independently —CH₂F. In embodiments, R^{3D} is independently —CH₂I. In embodiments, R^{3D} is independently —CN. In embodiments, R^{3D} is independently —OH. In embodiments, R^{3D} is independently —NH₂. In embodiments, R^{3D} is independently -COOH. In embodiments, R^{3D} is independently —CONH₂. In embodiments, R^{3D} is independently —OCCl₃. In embodiments, R^{3D} is independently —OCF₃. In embodiments, R^{3D} is independently —OCBr₃. In embodiments, R^{3D} is independently —OCI₃. In embodiments, R^{3D} is independently —OCHCl₂. In embodiments, R^{3D} is independently —OCHBr₂. In embodiments, R^{3D} is independently —OCHI2. In embodiments, R^{3D} is independently —OCHF₂. In embodiments, R^{3D} is independently —OCH₂Cl. In embodiments, R^{3D} is independently —OCH2Br. In embodiments, R^{3D} is independently —OCH₂I. In embodiments, R^{3D} is independently —OCH₂F. In embodiments, R^{3D} is independently halogen. In embodiments, R^{3D} is independently —NO₂. In embodiments, R^{3D} is independently —OCH₃. In embodiments, R^{3D} is independently —OCH₂CH₃. In embodiments, R^{3D} is independently —OCH(CH₃)₂. In embodiments, R^{3D} is independently-OC(CH₃)₃. In embodiments, R^{3D} is independently —CH₃. In embodiments, R^{3D} is independently-CH₂CH₃. In embodiments, R^{3D} is independently —CH(CH₃)₂. In embodiments, R^{3D} is independently —C(CH₃)₃. In embodiments, R^{3D} is independently unsubstituted cyclopropyl. In embodiments, R^{3D} is independently unsubstituted cyclobutyl. In embodiments, R^{3D} is independently unsubstituted cyclopentyl. In embodiments, R^{3D} is independently unsubstituted cyclohexyl. In embodiments, R^{3D} is independently substituted or unsubstituted alkyl (e.g., C₁—C₈, C₁—C₆, or C₁—C₄). In embodiments, R^{3D} is independently substituted or unsubstituted heteroalkyl (e.g., 2 to 8 membered, 2 to 6 membered, or 2 to 4 membered). In embodiments, R^{3D} is independently substituted or unsubstituted cycloalkyl (e.g., C₃—C₈, C₃—C₆, or C₅—C₆). In embodiments, R^{3D} is independently substituted or unsubstituted heterocycloalkyl (e.g., 3 to 8 membered, 3 to 6 membered, or 5 to 6 membered). In embodiments, R^{3D} is independently substituted or unsubstituted aryl (e.g., C₆-C₁₀, C₁₀, or phenyl). In embodiments, R^{3D} is independently substituted or unsubstituted heteroaryl (e.g., 5 to 10 membered, 5 to 9 membered, or 5 to 6 membered).

R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D}.

In embodiments, R⁴ is independently hydrogen. In embodiments, R⁴ is independently halogen. In embodiments, R⁴ is independently —CX⁴₃. In embodiments, R⁴ is independently —CHX⁴₂. In embodiments, R⁴ is independently —CH₂X⁴. In embodiments, R⁴ is independently —OCX⁴₃. In embodiments, R⁴ is independently —OCH₂X⁴. In embodiments, R⁴ is independently —OCHX⁴₂. In embodiments, R⁴ is independently —CN. In embodiments, R⁴ is independently —SR^{4D}. In embodiments, R⁴ is independently —NR^{4A}R^{4B}. In embodiments, R⁴ is independently —OR^{4D}.

In embodiments, R⁴ is independently —CF₃. In embodiments, R⁴ is independently halogen. In embodiments, R⁴ is independently —CCl₃. In embodiments, R⁴ is independently —CBr₃. In embodiments, R⁴ is independently —CF₃. In embodiments, R⁴ is independently —CI₃. In embodiments, R⁴ is independently —CHCl₂. In embodiments, R⁴ is independently —CHBr₂. In embodiments, R⁴ is independently —CHF₂. In embodiments, R⁴ is independently —CHI2. In embodiments, R⁴ is independently —CH₂Cl. In embodiments, R⁴ is independently —CH₂Br. In embodiments, R⁴ is independently —CH₂F. In embodiments, R⁴ is independently —CH₂I. In embodiments, R⁴ is independently —CN. In embodiments, R⁴ is independently —OH. In embodiments, R⁴ is independently —NH₂. In embodiments, R⁴ is independently —SH. In embodiments, R⁴ is independently —OCCl₃. In embodiments, R⁴ is independently —OCF₃. In embodiments, R⁴ is independently —OCBr₃. In embodiments, R⁴ is independently —OCI₃. In embodiments, R⁴ is independently —OCHCl₂. In embodiments, R⁴ is independently —OCHBr₂. In embodiments, R⁴ is independently —OCHI2. In embodiments, R⁴ is independently —OCHF₂. In embodiments, R⁴ is independently —OCH₂Cl. In embodiments, R⁴ is independently —OCH₂Br. In embodiments, R⁴ is independently —OCH₂I. In embodiments, R⁴ is independently —OCH₂F. In embodiments, R⁴ is independently —F. In embodiments, R⁴ is independently —Cl. In embodiments, R⁴ is independently —Br. In embodiments, R⁴ is independently —I. In embodiments, R⁴ is independently —SCH₃. In embodiments, R⁴ is independently —OCH₃. In embodiments, R⁴ is independently —SCH₂CH₃. In embodiments, R⁴ is independently —OCH₂CH₃. In embodiments, R⁴ is independently -OCH₂CH₃. In embodiments, R⁴ is independently —OCH(CH₃)₂. In embodiments, R⁴ is independently —OC(CH₃)₃.

R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered),; R^{4A} and R^{4B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered).

In embodiments, R^{4A}, R^{4B}, R^{4C}, and R^{4D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, or unsubstituted methyl.

In embodiments, R^{4A} is independently unsubstituted C₁—C₄ alkyl. In embodiments, R^{4A} is independently unsubstituted cyclopropyl. In embodiments, R^{4A} is independently unsubstituted phenyl. In embodiments, R^{4A} is independently hydrogen. In embodiments, R^{4A} is independently —CCl₃. In embodiments, R^{4A} is independently —CBr₃. In embodiments, R^{4A} is independently —CF₃. In embodiments, R^{4A} is independently —CI₃. In embodiments, R^{4A} is independently —CHCl₂. In embodiments, R^{4A} is independently —CHBr₂. In embodiments, R^{4A} is independently —CHF₂. In embodiments, R^{4A} is independently —CHI2. In embodiments, R^{4A} is independently —CH₂Cl. In embodiments, R^{4A} is independently —CH₂Br. In embodiments, R^{4A} is independently —CH₂F. In embodiments, R^{4A} is independently —CH₂I. In embodiments, R^{4A} is independently —CN. In embodiments, R^{4A} is independently —OH. In embodiments, R^{4A} is independently —COOH. In embodiments, R^{4A} is independently —CONH₂. In embodiments, R^{4A} is independently —CH₃. In embodiments, R^{4A} is independently —CH₂CH₃. In embodiments, R^{4A} is independently —CH(CH₃)₂. In embodiments, R^{4A} is independently -C(CH₃)₃. In embodiments, R^{4A} is independently unsubstituted cyclopropyl. In embodiments, R^{4A} is independently unsubstituted cyclobutyl. In embodiments, R^{4A} is independently unsubstituted cyclopentyl. In embodiments, R^{4A} is independently unsubstituted cyclohexyl. In embodiments, R^{4A} is independently substituted or unsubstituted alkyl (e.g., C₁—C₈, C₁—C₆, or C₁—C₄). In embodiments, R^{4A} is independently substituted or unsubstituted heteroalkyl (e.g., 2 to 8 membered, 2 to 6 membered, or 2 to 4 membered). In embodiments, R^{4A} is independently substituted or unsubstituted cycloalkyl (e.g., C₃—C₈, C₃-C₆, or C₅—C₆). In embodiments, R^{4A} is independently substituted or unsubstituted heterocycloalkyl (e.g., 3 to 8 membered, 3 to 6 membered, or 5 to 6 membered). In embodiments, R^{4A} is independently substituted or unsubstituted aryl (e.g., C₆—C₁₀, C₁₀, or phenyl). In embodiments, R^{4A} is independently substituted or unsubstituted heteroaryl (e.g., 5 to 10 membered, 5 to 9 membered, or 5 to 6 membered). In embodiments, R^{4A} is independently unsubstituted alkyl (e.g., C₁—C₈, C₁—C₆, or C₁—C₄). In embodiments, R^{4A} is independently unsubstituted heteroalkyl (e.g., 2 to 8 membered, 2 to 6 membered, or 2 to 4 membered). In embodiments, R^{4A} is independently unsubstituted cycloalkyl (e.g., C₃—C₈, C₃-C₆, or C₅—C₆). In embodiments, R^{4A} is independently unsubstituted heterocycloalkyl (e.g., 3 to 8 membered, 3 to 6 membered, or 5 to 6 membered). In embodiments, R^{4A} is independently unsubstituted aryl (e.g., C₆—C₁₀, C₁₀, or phenyl). In embodiments, R^{4A} is independently unsubstituted heteroaryl (e.g., 5 to 10 membered, 5 to 9 membered, or 5 to 6 membered).

In embodiments, R^{4B} is independently unsubstituted C₁—C₄ alkyl. In embodiments, R^{4B} is independently unsubstituted cyclopropyl. In embodiments, R^{4B} is independently unsubstituted phenyl. In embodiments, R^{4B} is independently hydrogen. In embodiments, R^{4B} is independently —CCl₃. In embodiments, R^{4B} is independently —CBr₃. In embodiments, R^{4B} is independently —CF₃. In embodiments, R^{4B} is independently —CI₃. In embodiments, R^{4B} is independently —CHCl₂. In embodiments, R^{4B} is independently —CHBr₂. In embodiments, R^{4B} is independently —CHF₂. In embodiments, R^{4B} is independently —CHI2. In embodiments, R^{4B} is independently —CH₂Cl. In embodiments, R^{4B} is independently —CH₂Br. In embodiments, R^{4B} is independently —CH₂F. In embodiments, R^{4B} is independently —CH₂I. In embodiments, R^{4B} is independently —CN. In embodiments, R^{4B} is independently —OH. In embodiments, R^{4B} is independently —COOH. In embodiments, R^{4B} is independently —CONH₂. In embodiments, R^{4B} is independently —CH₃. In embodiments, R^{4B} is independently —CH₂CH₃. In embodiments, R^{4B} is independently —CH(CH₃)₂. In embodiments, R^{4B} is independently -C(CH₃)₃. In embodiments, R^{4B} is independently unsubstituted cyclopropyl. In embodiments, R^{4B} is independently unsubstituted cyclobutyl. In embodiments, R^{4B} is independently unsubstituted cyclopentyl. In embodiments, R^{4B} is independently unsubstituted cyclohexyl. In embodiments, R^{4B} is independently substituted or unsubstituted alkyl (e.g., C₁—C₈, C₁—C₆, or C₁—C₄). In embodiments, R^{4B} is independently substituted or unsubstituted heteroalkyl (e.g., 2 to 8 membered, 2 to 6 membered, or 2 to 4 membered). In embodiments, R^{4B} is independently substituted or unsubstituted cycloalkyl (e.g., C₃—C₈, C₃-C₆, or C₅—C₆). In embodiments, R^{4B} is independently substituted or unsubstituted heterocycloalkyl (e.g., 3 to 8 membered, 3 to 6 membered, or 5 to 6 membered). In embodiments, R^{4B} is independently substituted or unsubstituted aryl (e.g., C₆—C₁₀, C₁₀, or phenyl). In embodiments, R^{4B} is independently substituted or unsubstituted heteroaryl (e.g., 5 to 10 membered, 5 to 9 membered, or 5 to 6 membered). In embodiments, R^{4B} is independently unsubstituted alkyl (e.g., C₁—C₈, C₁—C₆, or C₁—C₄). In embodiments, R^{4B} is independently unsubstituted heteroalkyl (e.g., 2 to 8 membered, 2 to 6 membered, or 2 to 4 membered). In embodiments, R^{4B} is independently unsubstituted cycloalkyl (e.g., C₃—C₈, C₃-C₆, or C₅—C₆). In embodiments, R^{4B} is independently unsubstituted heterocycloalkyl (e.g., 3 to 8 membered, 3 to 6 membered, or 5 to 6 membered). In embodiments, R^{4B} is independently unsubstituted aryl (e.g., C₆—C₁₀, C₁₀, or phenyl). In embodiments, R^{4B} is independently unsubstituted heteroaryl (e.g., 5 to 10 membered, 5 to 9 membered, or 5 to 6 membered).

In embodiments, R^{4C} is independently unsubstituted C₁—C₄ alkyl. In embodiments, R^{4C} is independently unsubstituted cyclopropyl. In embodiments, R^{4C} is independently unsubstituted phenyl. In embodiments, R^{4C} is independently hydrogen. In embodiments, R^{4C} is independently —CCl₃. In embodiments, R^{4C} is independently —CBr₃. In embodiments, R^{4C} is independently —CF₃. In embodiments, R^{4C} is independently —CI₃. In embodiments, R^{4C} is independently —CHCl₂. In embodiments, R^{4C} is independently —CHBr₂. In embodiments, R^{4C} is independently —CHF₂. In embodiments, R^{4C} is independently —CHI2. In embodiments, R^{4C} is independently —CH₂Cl. In embodiments, R^{4C} is independently —CH₂Br. In embodiments, R^{4C} is independently —CH₂F. In embodiments, R^{4C} is independently —CH₂I. In embodiments, R^{4C} is independently —CN. In embodiments, R^{4C} is independently —OH. In embodiments, R^{4C} is independently —COOH. In embodiments, R^{4C} is independently —CONH₂. In embodiments, R^{4C} is independently —CH₃. In embodiments, R^{4C} is independently —CH₂CH₃. In embodiments, R^{4C} is independently —CH(CH₃)₂. In embodiments, R^{4C} is independently -C(CH₃)₃. In embodiments, R^{4C} is independently unsubstituted cyclopropyl. In embodiments, R^{4C} is independently unsubstituted cyclobutyl. In embodiments, R^{4C} is independently unsubstituted cyclopentyl. In embodiments, R^{4C} is independently unsubstituted cyclohexyl. In embodiments, R^{4C} is independently substituted or unsubstituted alkyl (e.g., C₁—C₈, C₁—C₆, or C₁—C₄). In embodiments, R^{4C} is independently substituted or unsubstituted heteroalkyl (e.g., 2 to 8 membered, 2 to 6 membered, or 2 to 4 membered). In embodiments, R^{4C} is independently substituted or unsubstituted cycloalkyl (e.g., C₃—C₈, C₃-C₆, or C₅—C₆). In embodiments, R^{4C} is independently substituted or unsubstituted heterocycloalkyl (e.g., 3 to 8 membered, 3 to 6 membered, or 5 to 6 membered). In embodiments, R^{4C} is independently substituted or unsubstituted aryl (e.g., C₆—C₁₀, C₁₀, or phenyl). In embodiments, R^{4C} is independently substituted or unsubstituted heteroaryl (e.g., 5 to 10 membered, 5 to 9 membered, or 5 to 6 membered). In embodiments, R^{4C} is independently unsubstituted alkyl (e.g., C₁—C₈, C₁—C₆, or C₁—C₄). In embodiments, R^{4C} is independently unsubstituted heteroalkyl (e.g., 2 to 8 membered, 2 to 6 membered, or 2 to 4 membered). In embodiments, R^{4C} is independently unsubstituted cycloalkyl (e.g., C₃—C₈, C₃-C₆, or C₅—C₆). In embodiments, R^{4C} is independently unsubstituted heterocycloalkyl (e.g., 3 to 8 membered, 3 to 6 membered, or 5 to 6 membered). In embodiments, R^{4C} is independently unsubstituted aryl (e.g., C₆—C₁₀, C₁₀, or phenyl). In embodiments, R^{4C} is independently unsubstituted heteroaryl (e.g., 5 to 10 membered, 5 to 9 membered, or 5 to 6 membered).

In embodiments, R^{4D} is independently unsubstituted C₁—C₄ alkyl. In embodiments, R^{4D} is independently unsubstituted cyclopropyl. In embodiments, R^{4D} is independently unsubstituted phenyl. In embodiments, R^{4D} is independently hydrogen. In embodiments, R^{4D} is independently —CCl₃. In embodiments, R^{4D} is independently —CBr₃. In embodiments, R^{4D} is independently —CF₃. In embodiments, R^{4D} is independently —CI₃. In embodiments, R^{4D} is independently —CHCl₂. In embodiments, R^{4D} is independently —CHBr₂. In embodiments, R^{4D} is independently —CHF₂. In embodiments, R^{4D} is independently —CHI2. In embodiments, R^{4D} is independently —CH₂Cl. In embodiments, R^{4D} is independently —CH₂Br. In embodiments, R^{4D} is independently —CH₂F. In embodiments, R^{4D} is independently —CH₂I. In embodiments, R^{4D} is independently —CN. In embodiments, R^{4D} is independently —OH. In embodiments, R^{4D} is independently -COOH. In embodiments, R^{4D} is independently —CONH₂. In embodiments, R^{4D} is independently —CH₃. In embodiments, R^{4D} is independently —CH₂CH₃. In embodiments, R^{4D} is independently —CH(CH₃)₂. In embodiments, R^{4D} is independently -C(CH₃)₃. In embodiments, R^{4D} is independently unsubstituted cyclopropyl. In embodiments, R^{4D} is independently unsubstituted cyclobutyl. In embodiments, R^{4D} is independently unsubstituted cyclopentyl. In embodiments, R^{4D} is independently unsubstituted cyclohexyl. In embodiments, R^{4D} is independently substituted or unsubstituted alkyl (e.g., C₁—C₈, C₁—C₆, or C₁—C₄). In embodiments, R^{4D} is independently substituted or unsubstituted heteroalkyl (e.g., 2 to 8 membered, 2 to 6 membered, or 2 to 4 membered). In embodiments, R^{4D} is independently substituted or unsubstituted cycloalkyl (e.g., C₃—C₈, C₃-C₆, or C₅—C₆). In embodiments, R^{4D} is independently substituted or unsubstituted heterocycloalkyl (e.g., 3 to 8 membered, 3 to 6 membered, or 5 to 6 membered). In embodiments, R^{4D} is independently substituted or unsubstituted aryl (e.g., C₆—C₁₀, C₁₀, or phenyl). In embodiments, R^{4D} is independently substituted or unsubstituted heteroaryl (e.g., 5 to 10 membered, 5 to 9 membered, or 5 to 6 membered). In embodiments, R^{4D} is independently unsubstituted alkyl (e.g., C₁—C₈, C₁—C₆, or C₁—C₄). In embodiments, R^{4D} is independently unsubstituted heteroalkyl (e.g., 2 to 8 membered, 2 to 6 membered, or 2 to 4 membered). In embodiments, R^{4D} is independently unsubstituted cycloalkyl (e.g., C₃—C₈, C₃-C₆, or C₅—C₆). In embodiments, R^{4D} is independently unsubstituted heterocycloalkyl (e.g., 3 to 8 membered, 3 to 6 membered, or 5 to 6 membered). In embodiments, R^{4D} is independently unsubstituted aryl (e.g., C₆—C₁₀, C₁₀, or phenyl). In embodiments, R^{4D} is independently unsubstituted heteroaryl (e.g., 5 to 10 membered, 5 to 9 membered, or 5 to 6 membered).

In embodiments, L¹ is substituted or unsubstituted heteroalkylene and R¹ is independently substituted or unsubstituted aryl or substituted or unsubstituted heteroaryl.

In embodiments, L¹ is —C(O)N(R^{L1})—(C₁—C₆ alkyl)- or —SO₂N(R^{L1})—(C₁—C₆ alkyl)-; R¹ is independently substituted phenyl or substituted 5 to 6 membered heteroaryl; and R^{L1} is independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, unsubstituted alkyl, or unsubstituted cycloalkyl.

In embodiments, L¹ is —C(O)N(R^{L1})—(C₁—C₆ alkyl)- or —SO₂N(R^{L1})—(C₁—C₆ alkyl)-; R¹ is independently substituted phenyl or substituted 5 to 6 membered heteroaryl; and R^{L1} is independently hydrogen, unsubstituted C₁—C₆ alkyl, or unsubstituted C₃—C₆ cycloalkyl.

In embodiments, L¹ is —C(O)N(R^{L1})CH₂— or —SO₂N(R^{L1})CH₂—; R¹ is independently substituted phenyl or substituted 5 to 6 membered heteroaryl; and R^{L1} is independently hydrogen, unsubstituted methyl, unsubstituted ethyl, unsubstituted isopropyl, or unsubstituted cyclopropyl.

In embodiments, L¹ is —C(O)N(R^{L1})—; R¹ is independently substituted phenyl or substituted 5 to 6 membered heteroaryl; and R^{L1} is independently hydrogen.

In embodiments, R¹ is independently R¹⁰-substituted phenyl or R¹⁰-substituted 5 to 6 membered heteroaryl; R¹⁰ is independently halogen, —CX¹⁰₃, —CHX¹⁰₂, —CH₂X¹⁰, —OCX¹⁰₃, —OCH₂X¹⁰, —OCHX¹⁰₂, —CN, —SO₂R^{10D}, —SR^{10D}, —C(O)R^{10C}, —OR^{10D}, substituted or unsubstituted C₁—C₆ alkyl, substituted or unsubstituted 2 to 6 membered heteroalkyl, substituted or unsubstituted C₃—C₆ cycloalkyl, substituted or unsubstituted 3 to 6 membered heterocycloalkyl, substituted or unsubstituted phenyl, or substituted or unsubstituted 5 to 6 membered heteroaryl; R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, unsubstituted C₁—C₆ alkyl, or unsubstituted C₃—C₆ cycloalkyl; and X¹⁰ is independently —F, —Cl, —Br, or —I.

In embodiments, R¹ is independently R¹⁰-substituted phenyl or R¹⁰-substituted 5 to 6 membered heteroaryl; R¹⁰ is independently halogen, —CX¹⁰₃, —CHX¹⁰₂, —CH₂X¹⁰, —OCX¹⁰₃, —OCH₂X¹⁰, —OCHX¹⁰₂, —CN, —SO₂R^{10D}, —SR^{10D}, —OR^{10D}, unsubstituted C₁—C₄ alkyl, unsubstituted 2 to 6 membered heteroalkyl, unsubstituted C₃-C₄ cycloalkyl, or unsubstituted 3 to 6 membered heterocycloalkyl; R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, or unsubstituted methyl; and X¹⁰ is independently —F, —Cl, —Br, or —I.

In embodiments, R¹ is independently R¹⁰-substituted phenyl or R¹⁰-substituted 5 to 6 membered heteroaryl; and R¹⁰ is independently halogen, —CF₃, —CHF₂, —CH₂F, —OCF₃, —OCH₂F, —OCHF₂, —OCH₃, —CH₂OCH₃, —CN, —SO₂CH₃, —SCH₃, —OCH₃, unsubstituted C₁—C₄ alkyl, or unsubstituted 3 to 6 membered heterocycloalkyl.

In embodiments, R¹ is independently

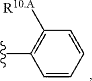

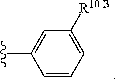

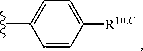

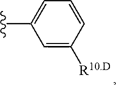

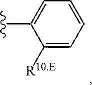

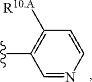

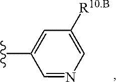

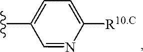

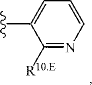

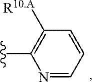

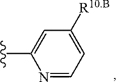

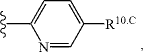

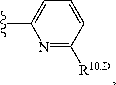

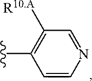

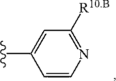

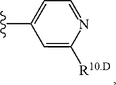

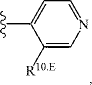

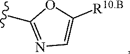

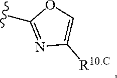

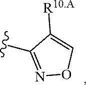

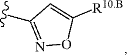

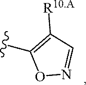

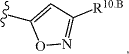

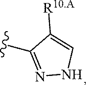

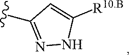

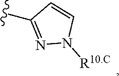

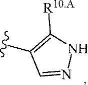

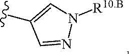

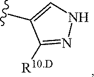

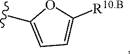

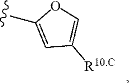

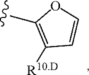

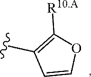

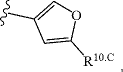

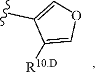

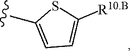

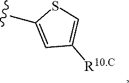

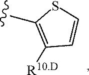

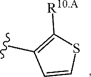

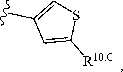

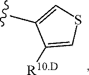

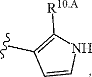

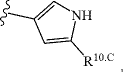

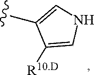

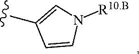

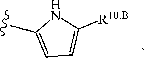

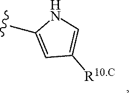

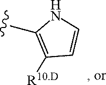

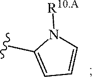

and R^{10.A}, R^{10.B} , R^{10C}, R^{10.D}, and R^{10.E} are independently —F, —Cl, —CH₃, —OCH₃, —OH, unsubstituted morpholinyl, or unsubstituted piperazinyl.

In embodiments, L¹ is a bond; R¹ is independently —SO₂NR^{1A}R^{1B}, —NR^{1A}R^{1B}, or —C(O)NR^{1A}R^{1B}; and R^{1A} and R^{1B} are independently hydrogen, substituted or unsubstituted alkyl, substituted or unsubstituted cycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl.

In embodiments, L¹ is a bond; R¹ is independently —SO₂NR^{1A}R^{1B} or —C(O)NR^{1A}R^{1B}; R^{1A} and R^{1B} are independently hydrogen, substituted or unsubstituted C₁—C₆ alkyl, substituted or unsubstituted C₃—C₆ cycloalkyl, substituted or unsubstituted phenyl, or substituted or unsubstituted 5 to 6 membered heteroaryl; R^{1A} and R^{1B} substituents bonded to the same nitrogen atom may optionally be joined to form a substituted or unsubstituted C₃—C₆ heterocycloalkyl.

In embodiments, L¹ is a bond; R¹ is independently —C(O)NR^{1A}R^{1B}; R^{1A} is independently hydrogen, unsubstituted C₁—C₄ alkyl, or unsubstituted cyclopropyl; and R^{1B} are independently substituted or unsubstituted phenyl or substituted or unsubstituted 5 to 6 membered heteroaryl.

In embodiments, L¹ is a bond; R¹ is independently —C(O)NR^{1A}R^{1B}; R^{1A} is independently hydrogen, unsubstituted C₁—C₄ alkyl, or unsubstituted cyclopropyl; R^{1B} is independently R¹⁰-substituted phenyl or R¹⁰-substituted 5 to 6 membered heteroaryl; R¹⁰ is independently halogen, —CX¹⁰₃, —CHX¹⁰₂, —CH₂X¹⁰, —OCX¹⁰₃, —OCH₂X¹⁰, —OCHX¹⁰₂, —SR^{10D}, —OR^{10D}, unsubstituted C₁—C₄ alkyl, unsubstituted 5 to 6 membered heterocycloalkyl; R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, or unsubstituted methyl; and X¹⁰ is independently —F, —Cl, —Br, or —I.

In embodiments, L¹ is a bond; R¹ is independently —C(O)NR^{1A}R^{1B}; R^{1A} is independently hydrogen, unsubstituted C₁—C₄ alkyl, or unsubstituted cyclopropyl; R^{1B} is independently R¹⁰-substituted phenyl or R¹⁰-substituted 5 to 6 membered heteroaryl; and R¹⁰ is independently halogen, —CF₃, —CHF₂, —CH₂F, —OCF₃, —OCH₂F, —OCHF₂, —OCH₃, —SCH₃, —OCH₃, unsubstituted C₁—C₄ alkyl, unsubstituted morpholinyl, or unsubstituted piperazinyl.

In embodiments, L¹ is a bond; R¹ is independently —C(O)NR^{1A}R^{1B}; R^{1A} is independently hydrogen, unsubstituted C₁—C₄ alkyl, or unsubstituted cyclopropyl; R^{1B} is independently

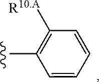

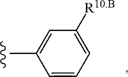

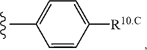

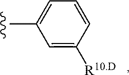

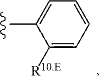

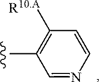

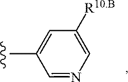

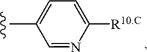

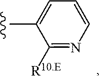

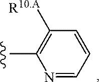

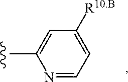

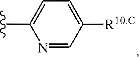

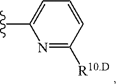

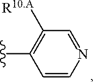

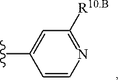

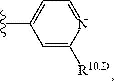

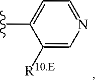

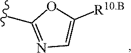

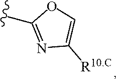

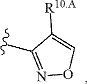

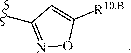

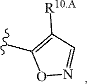

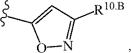

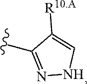

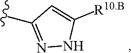

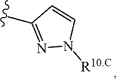

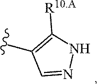

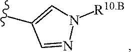

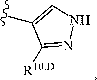

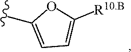

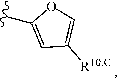

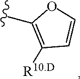

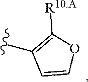

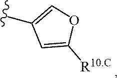

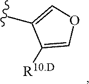

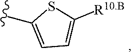

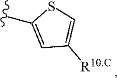

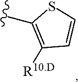

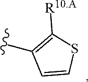

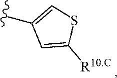

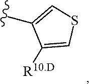

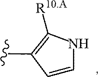

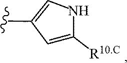

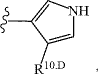

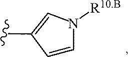

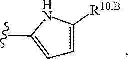

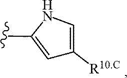

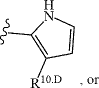

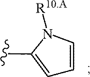

and R^{10.A}, R^{10.B}, R^{10C}, R^{10.D}, and R^{10.E} are independently —F, —Cl, —CH₃, —OCH₃, —OH, unsubstituted morpholinyl, or unsubstituted piperazinyl.

In embodiments, L¹ is a bond; R¹ is independently —C(O)NR^{1A}R^{1B}; R^{1A} is independently hydrogen, unsubstituted C₁—C₄ alkyl, or unsubstituted cyclopropyl; R^{1B} is independently

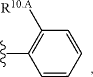

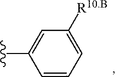

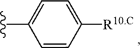

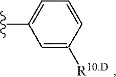

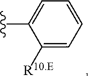

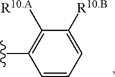

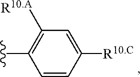

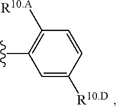

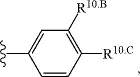

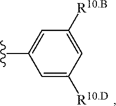

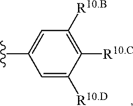

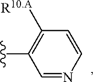

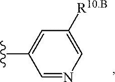

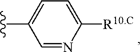

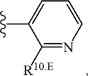

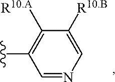

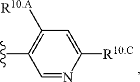

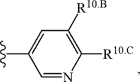

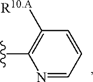

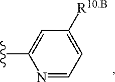

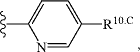

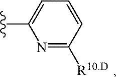

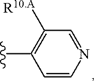

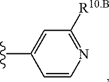

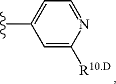

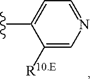

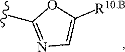

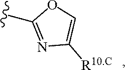

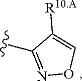

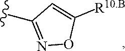

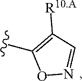

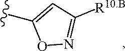

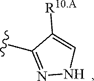

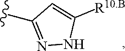

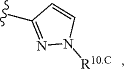

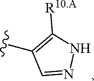

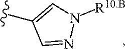

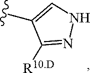

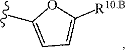

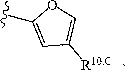

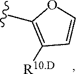

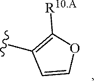

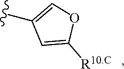

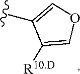

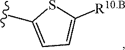

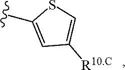

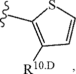

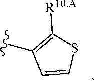

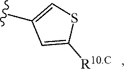

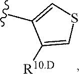

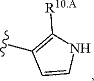

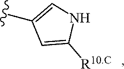

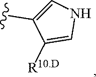

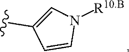

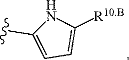

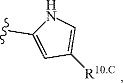

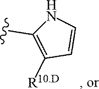

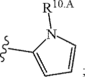

and R^{10.A}, R^{10.B} , R^{10.C}, R^{10.D}, and R^{10.}^{E} are independently —F, —Cl, —CH₃, —OCH₃, —OH, substituted or unsubstituted morpholinyl, or substituted or unsubstituted piperazinyl.

In embodiments, L¹ is a bond; R¹ is independently —C(O)NR^{1A}R^{1B}; R^{1A} is independently hydrogen, unsubstituted C₁—C₄ alkyl, or unsubstituted cyclopropyl; R^{1B} is independently

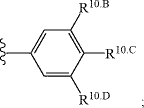

and R^{10.B}, R^{10C}, and R^{10.D} are independently —F, —Cl, —CH₃, —OCH₃, —OH, substituted or unsubstituted morpholinyl, or substituted or unsubstituted piperazinyl.

In embodiments, L¹ is a bond; R¹ is independently -SO₂NR^{1A}R^{1B} or —C(O)NR^{1A}R^{1B}; and R^{1A} and R^{1B} bonded to the same nitrogen atom are joined to form a substituted or unsubstituted C₃—C₆ heterocycloalkyl.

In embodiments, L¹ is a bond; R¹ is independently —C(O)NR^{1A}R^{1B}; and R^{1A} and R^{1B} bonded to the same nitrogen atom are joined to form a substituted or unsubstituted piperazinyl.

In embodiments, L¹ is —C(O)—; R¹ is independently —NR^{1A}R^{1B}; and R^{1A} and R^{1B} are independently hydrogen, substituted or unsubstituted alkyl, substituted or unsubstituted cycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl.

In embodiments, L¹ is —C(O)—; R¹ is independently -NR^{1A}R^{1B}; and R^{1A} and R^{1B} are independently hydrogen, substituted or unsubstituted C₁—C₆ alkyl, substituted or unsubstituted C₃—C₆ cycloalkyl, substituted or unsubstituted phenyl, or substituted or unsubstituted 5 to 6 membered heteroaryl; R^{1A} and R^{1B} substituents bonded to the same nitrogen atom may optionally be joined to form a substituted or unsubstituted C₃—C₆ heterocycloalkyl..

In embodiments, L¹ is —C(O)—; R¹ is independently —NR^{1A}R^{1B}; R^{1A} is independently hydrogen, unsubstituted C₁—C₄ alkyl, or unsubstituted cyclopropyl; R^{1B} is independently R¹⁰-substituted phenyl or R¹⁰-substituted 5 to 6 membered heteroaryl; R¹⁰ is independently halogen, —CX¹⁰₃, —CHX¹⁰₂, —CH₂X¹⁰, —OCX¹⁰₃, —OCH₂X¹⁰, —OCHX¹⁰₂, —SR^{10D}, —OR^{10D}, unsubstituted C₁—C₄ alkyl, unsubstituted 2 to 6 membered heteroalkyl, unsubstituted 5 to 6 membered heterocycloalkyl; R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, or unsubstituted methyl; and X¹⁰ is independently —F, —Cl, —Br, or —I.

In embodiments, L¹ is —C(O)—; R¹ is independently —NR^{1A}R^{1B}; R^{1A} is independently hydrogen, unsubstituted C₁—C₄ alkyl, or unsubstituted cyclopropyl; R^{1B} is independently

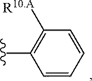

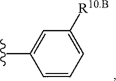

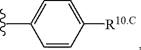

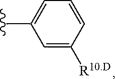

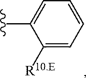

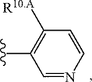

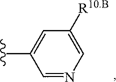

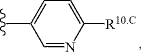

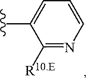

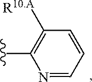

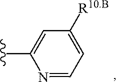

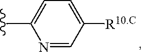

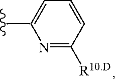

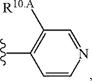

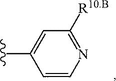

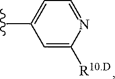

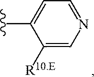

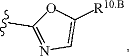

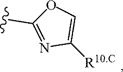

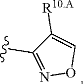

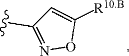

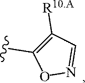

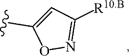

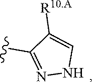

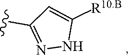

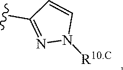

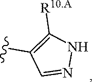

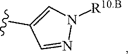

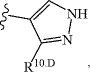

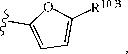

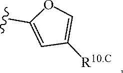

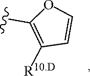

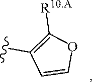

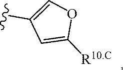

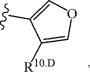

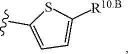

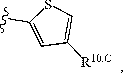

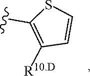

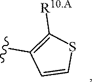

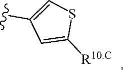

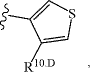

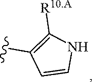

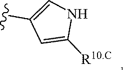

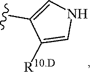

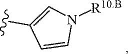

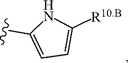

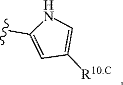

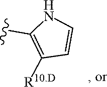

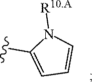

and R^{10.A}, R^{10.B}, R^{10C}, R^{10.D}, and R^{10.E} are independently —F, —Cl, —CH₃, —OCH₃, —OH, unsubstituted morpholinyl, or unsubstituted piperazinyl.

In embodiments, L¹ is —C(O)—; R¹ is independently —NR^{1A}R^{1B}; R^{1A} is independently hydrogen, unsubstituted C₁—C₄ alkyl, or unsubstituted cyclopropyl; R^{1B} is independently

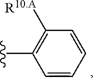

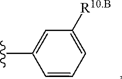

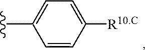

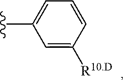

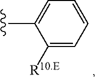

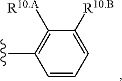

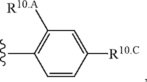

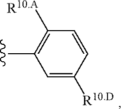

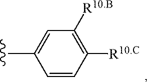

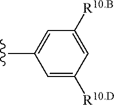

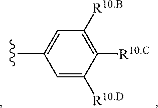

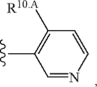

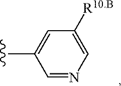

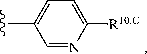

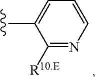

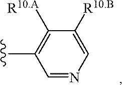

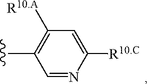

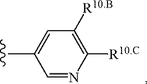

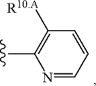

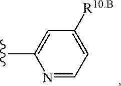

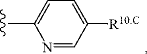

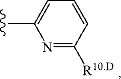

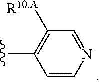

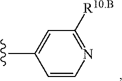

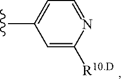

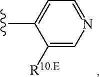

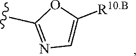

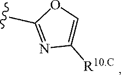

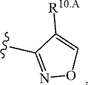

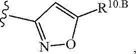

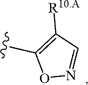

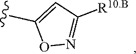

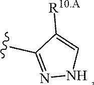

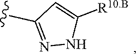

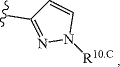

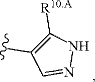

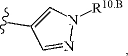

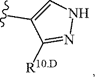

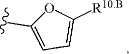

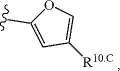

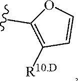

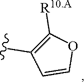

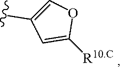

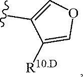

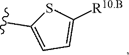

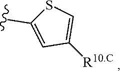

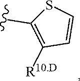

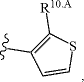

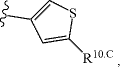

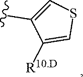

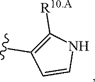

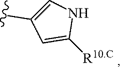

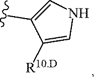

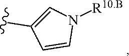

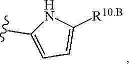

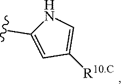

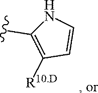

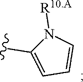

and R^{10.A}, R^{10.B} , R^{10.C,} R^{10.D}, and R^{10.E} are independently —F, —Cl, —CH₃, —OCH₃, —OH, substituted or unsubstituted morpholinyl, or substituted or unsubstituted piperazinyl.

In embodiments, L¹ is —C(O)—; R¹ is independently —NR^{1A}R^{1B}; R^{1A} is independently hydrogen, unsubstituted C₁—C₄ alkyl, or unsubstituted cyclopropyl; R^{1B} is independently

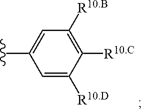 [SMILES: O=C(Nc1ncccc1O)c1c(O)c2c(n(Cc3ccco3)c1=O)CCCC2]

and R^{10.A}, R^{10.B}, R^{10.C}, R^{10.D}, and R^{10.E} are independently —F, —Cl, —CH₃, —OCH₃, —OH, substituted or unsubstituted morpholinyl, or substituted or unsubstituted piperazinyl.

In embodiments, L¹ is —C(O)—; R¹ is independently —NR^{1A}R^{1B}; and R^{1A} and R^{1B} bonded to the same nitrogen atom are joined to form a substituted or unsubstituted C₃—C₆ heterocycloalkyl. In embodiments, L¹ is —C(O)—; R¹ is independently —NR^{1A}R^{1B}; and R^{1A} and R^{1B} bonded to the same nitrogen atom are joined to form a substituted or unsubstituted piperazinyl.

In embodiments, R⁴ is independently -SR^{4D}, -NR^{4A}R^{4B}, or -OR^{4D}; and R^{4A}, R^{4B}, and R^{4D} are independently hydrogen or unsubstituted C₁—C₆ alkyl; R^{4A} and R^{4B} substituents bonded to the same nitrogen atom may optionally be joined to form a substituted or unsubstituted 5 to 6 membered heterocycloalkyl.

In embodiments, R⁴ is independently —OR^{4D}; and R^{4D} is independently hydrogen or unsubstituted C₁—C₆ alkyl.

In embodiments, R² is independently R²⁰-substituted phenyl or R²⁰-substituted 5 to 6 membered heteroaryl; R²⁰ is independently halogen, —CX²⁰₃, —CHX²⁰₂, —CH₂X²⁰, —OCX²⁰₃, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R^{20B}, —N(O)m20, —NR^{20A}R^{20B}, —C(O)R^{20C}, —C(O)—OR^{20C}, —C(O)NR^{20A}R^{20B}, —OR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCl₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl; X²⁰ is independently —F, —Cl, —Br, or —I; n20 is independently an integer from 0 to 4; and m20 and v20 are independently 1 or 2.

In embodiments, R² is independently R²⁰-substituted phenyl or R²⁰-substituted 5 to 6 membered heteroaryl; and R²⁰ is independently halogen.

In embodiments, R² is independently R²⁰-substituted phenyl or R²⁰-substituted 5 to 6 membered heteroaryl; and R²⁰ is independently —F.

In embodiments, L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, —N(R^{L1})CH₂—, —OCH₂—, —SCH₂—, —SO₂CH₂—, —C(O)CH₂—, —C(O)N(R^{L1})CH₂—, —N(R^{L1})C(O)CH₂—, —N(R^{L1})C(O)NHCH₂—, —NHC(O)N(R^{L1})CH₂—, —C(O)OCH₂—, —OC(O)CH₂—, —SO₂N(R^{L1})CH₂—, —N(R^{L1})SO₂CH₂—, —CH₂N(R^{L1})—, —CH₂O—, —CH₂S—, —CH₂SO₂—, —CH₂C(O)—, —CH₂C(O)N(R^{L}1)—, —CH₂N(R^{L1})C(O)—, —CH₂N(R^{L1})C(O)NH—, —CH₂NHC(O)N(R^{L1})—, —CH₂C(O)O—, —CH₂OC(O)—, —CH₂SO₂N(R^{L1})—, —CH₂N(R^{L1})SO₂—; and R^{L1} is independently hydrogen or unsubstituted C₁—C₄ alkyl.

In embodiments, L¹ is —C(O)N(R^{L1})— or —C(O)N(R^{L1})CH₂—; and R^{L1} is independently hydrogen or unsubstituted methyl.

In embodiments, R¹ is independently R¹⁰-substituted or unsubstituted C₃—C₆ cycloalkyl, R¹⁰-substituted or unsubstituted 3 to 6 membered heterocycloalkyl, R¹⁰-substituted or unsubstituted phenyl, or R¹⁰-substituted or unsubstituted 5 to 6 membered heteroaryl; R¹⁰ is independently halogen, —CX¹⁰₃, —CHX¹⁰₂, —CH₂X¹⁰, -OCX¹⁰₃, —OCH₂X¹⁰, —OCHX¹⁰₂, —SR^{10D}, —OR^{10D}, unsubstituted C₁—C₄ alkyl, unsubstituted 2 to 4 membered heteroalkyl, unsubstituted C₃—C₆ cycloalkyl, unsubstituted 3 to 6 membered heterocycloalkyl, unsubstituted phenyl, or unsubstituted 5 to 6 membered heteroaryl; R^{10D} is independently hydrogen or unsubstituted C₁—C₄ alkyl; and X¹⁰ is independently —F, —Cl, —Br, or —I.

In embodiments, R¹ is independently R¹⁰-substituted or unsubstituted C₃—C₆ cycloalkyl, R¹⁰-substituted or unsubstituted 3 to 6 membered heterocycloalkyl, R¹⁰-substituted or unsubstituted phenyl, or R¹⁰-substituted or unsubstituted 5 to 6 membered heteroaryl; and R¹⁰ is independently halogen, —OH, —OCH₃, —CH₃, unsubstituted 6 membered heterocycloalkyl.

In embodiments, the compound has the formula:

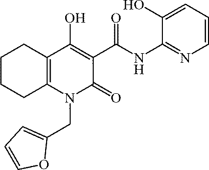 [SMILES: Cc1cccnc1NC(=O)c1c(O)c2ccccc2n(Cc2ccccc2)c1=O]

In embodiments, the salt (e.g., pharmaceutically acceptable salt) of the compound of formula VI is the HCl salt. In embodiments, the salt(e.g., pharmaceutically acceptable salt) of the compound of formula VI is the Cl⁻ salt. In embodiments, the salt (e.g., pharmaceutically acceptable salt) of the compound of formula VI is a pharmaceutically acceptable salt.

In embodiments, the compound has the formula:

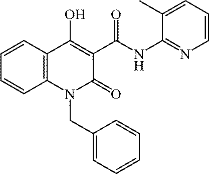 [SMILES: O=C(Nc1ccccn1)c1c(O)c2ccccc2n(Cc2ccccc2)c1=O]

In embodiments, the salt (e.g., pharmaceutically acceptable salt) of the compound of formula VII is the HCl salt. In embodiments, the salt (e.g., pharmaceutically acceptable salt) of the compound of formula VII is the Cl⁻ salt. In embodiments, the salt (e.g., pharmaceutically acceptable salt) of the compound of formula VII is a pharmaceutically acceptable salt.

In embodiments, the compound has the formula:

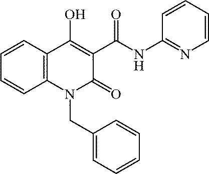 [SMILES: CC(C)Cn1c(=O)c(C(=O)Nc2cccc(F)c2)c(O)c2ccccc21]

In embodiments, the salt (e.g., pharmaceutically acceptable salt) of the compound of formula VIII is the HCl salt. In embodiments, the salt (e.g., pharmaceutically acceptable salt) of the compound of formula VIII is the Cl⁻ salt. In embodiments, the salt (e.g., pharmaceutically acceptable salt) of the compound of formula VIII is a pharmaceutically acceptable salt.

In embodiments, the compound has the formula:

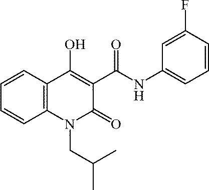 [SMILES: CC(C)Cn1c(=O)c(C(=O)Nc2cnn(C)c2)c(O)c2ccccc21]

In embodiments, the salt (e.g., pharmaceutically acceptable salt) of the compound of formula IX is the HCl salt. In embodiments, the salt (e.g., pharmaceutically acceptable salt) of the compound of formula IX is the Cl⁻ salt. In embodiments, the salt (e.g., pharmaceutically acceptable salt) of the compound of formula IX is a pharmaceutically acceptable salt.

In embodiments, the compound has the formula:

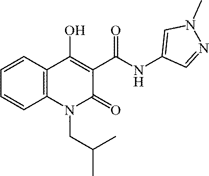 [SMILES: Cc1cccnc1NC(=O)c1c(O)c2ccccc2n(CC(C)C)c1=O ; ". In"]

In embodiments, the salt (e.g., pharmaceutically acceptable salt) of the compound of formula X is the HCl salt. In embodiments, the salt (e.g., pharmaceutically acceptable salt) of the compound of formula X is the Cl⁻ salt. In embodiments, the salt (e.g., pharmaceutically acceptable salt) of the compound of formula X is a pharmaceutically acceptable salt.

In embodiments, the compound has the formula:

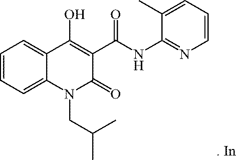 [SMILES: COc1ccc2c(c1)c(O)c(C(=O)Nc1ncccc1C)c(=O)n2CC(C)C ; ". In"]

embodiments, the compound has the formula:

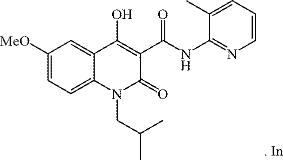 [SMILES: Cc1cc(NC(=O)c2c(O)c3ccccc3n(CC(C)C)c2=O)on1 ; "."]

embodiments, the compound has the formula:

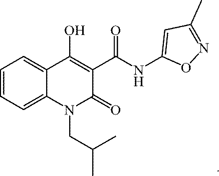 [SMILES: CC(C)Cn1c(=O)c(C(=O)Nc2ccccn2)c(O)c2ccccc21 ; "."]

In embodiments, the compound has the formula:

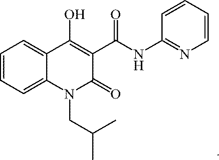 [SMILES: CC(C)Cn1c(=O)c(C(=O)Nc2ncccc2O)c(O)c2ccccc21 ; "."]

In embodiments, the compound has the formula:

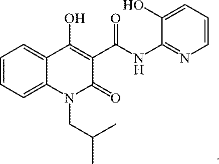 [SMILES: CC(C)Cn1c(=O)c(C(=O)Nc2cc(F)ccn2)c(O)c2ccccc21 ; "."]

In embodiments, the compound has the formula:

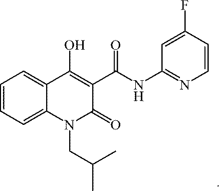 [SMILES: CC(C)Cn1c(=O)c(C(=O)Nc2ccon2)c(O)c2ccccc21 ; "."]

In embodiments, the compound has the formula:

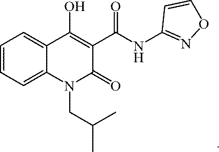 [SMILES: CC(C)Cn1c(=O)c(C(=O)Nc2cc(Cl)ccn2)c(O)c2ccccc21 ; "."]

In embodiments, the compound has the formula:

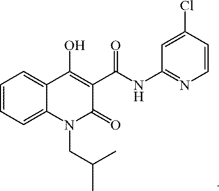 [SMILES: CC(C)Cn1c(=O)c(C(=O)Nc2cc(N3CCOCC3)ccn2)c(O)c2ccccc21 ; "."]

In embodiments, the compound has the formula:

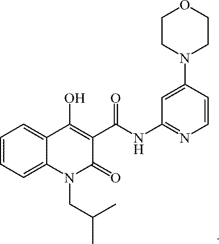 [SMILES: CC(C)Cn1c(=O)c(C(=O)Nc2ncco2)c(O)c2ccccc21 ; "."]

In embodiments, the compound has the formula:

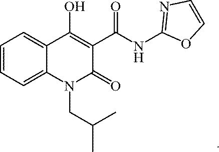 [SMILES: COc1ccnc(NC(=O)c2c(O)c3ccccc3n(CC(C)C)c2=O)c1 ; "."]

In embodiments, the compound has the formula:

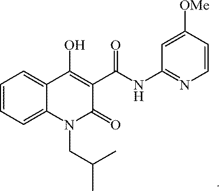 [SMILES: CC(C)Cn1c(=O)c(C(=O)Nc2ccn(C)n2)c(O)c2ccccc21 ; "."]

In embodiments, the compound has the formula:

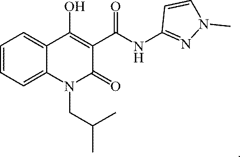 [SMILES: CC(C)Cn1c(=O)c(C(=O)Nc2cnn(C)c2)c(O)c2ccccc21 ; "."]

In embodiments, the compound has the formula:

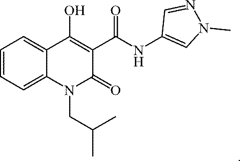 [SMILES: Cc1ccccc1NC(=O)c1c(O)c2ccccc2n(CC(C)C)c1=O ; "."]

In embodiments, the compound has the formula:

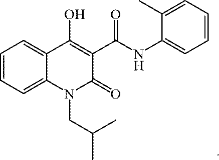 [SMILES: CC(C)Cn1c(=O)c(C(=O)Nc2ccccc2F)c(O)c2ccccc21 ; "."]

In embodiments, the compound has the formula:

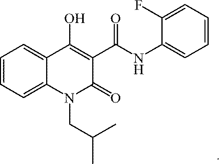 [SMILES: CC(C)Cn1c(=O)c(C(=O)Nc2cccc(F)c2)c(O)c2ccccc21 ; "."]

In embodiments, the compound has the formula:

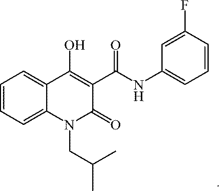 [SMILES: CC(C)Cn1c(=O)c(C(=O)Nc2ccc(F)cc2)c(O)c2ccccc21 ; "."]

In embodiments, the compound has the formula:

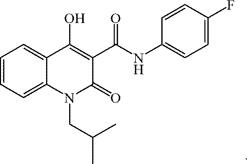 [SMILES: Cc1ncccc1NC(=O)c1c(O)c2ccccc2n(CC(C)C)c1=O ; "."]

In embodiments, the compound has the formula:

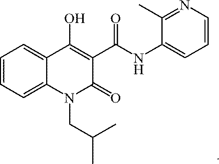 [SMILES: CC(C)Cn1c(=O)c(C(=O)NCc2ccco2)c(O)c2ccccc21 ; "."]

In embodiments, the compound has the formula:

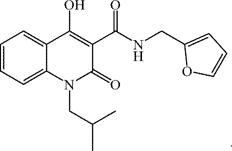 [SMILES: Cc1cc(NC(=O)c2c(O)c3ccccc3n(CC(C)C)c2=O)n[nH]1 ; "."]

In embodiments, the compound has the formula:

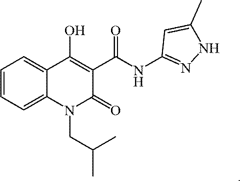 [SMILES: Cc1ccncc1NC(=O)c1c(O)c2ccccc2n(CC(C)C)c1=O ; "."]

In embodiments, the compound has the formula:

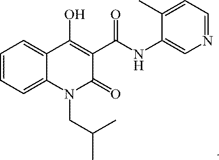 [SMILES: Cc1cnccc1NC(=O)c1c(O)c2ccccc2n(CC(C)C)c1=O ; "."]

In embodiments, the compound has the formula:

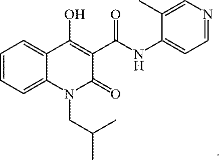 [SMILES: CC(C)Cn1c(=O)c(C(=O)Nc2cccc(N3CCNCC3)c2)c(O)c2ccccc21 ; "."]

In embodiments, the compound has the formula:

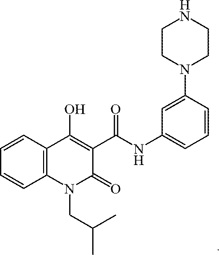 [SMILES: CC(C)Cn1c(=O)c(C(=O)Nc2cccc(N3CCOCC3)c2)c(O)c2ccccc21 ; "."]

In embodiments, the compound has the formula:

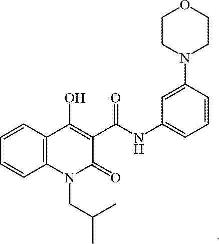 [SMILES: CC(C)Cn1c(=O)c(C(=O)Nc2cccc(N3CCN(C)CC3)c2)c(O)c2ccccc21 ; "."]

In embodiments, the compound has the formula:

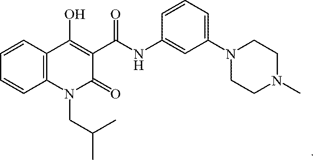 [SMILES: CC(C)Cn1c(=O)c(C(=O)Nc2cccc(N(C)C3CC3)c2)c(O)c2ccccc21 ; "."]

In embodiments, the compound has the formula:

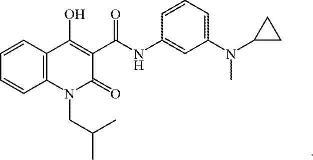 [SMILES: CC(C)Cn1c(=O)c(C(=O)Nc2cccc(N(C)CC3CC3)c2)c(O)c2ccccc21 ; "."]

In embodiments, the compound has the formula:

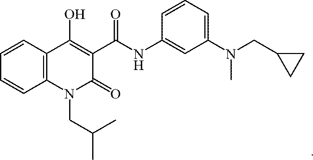 [SMILES: Cc1cccnc1NC(=O)c1c(O)c2c(Br)cccc2n(CC(C)C)c1=O ; "."]

In embodiments, the compound has the formula:

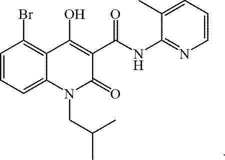 [SMILES: Cc1cccnc1NC(=O)c1c(O)c2ccc(Br)cc2n(CC(C)C)c1=O ; "."]

In embodiments, the compound has the formula:

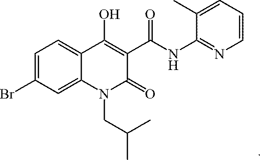 [SMILES: CC(C)Cn1c(=O)c(C(=O)Nc2cccc(F)c2)c(O)c2c(Br)cccc21 ; "."]

In embodiments, the compound has the formula:

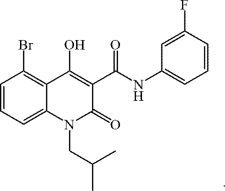

In embodiments, the compound has the formula:

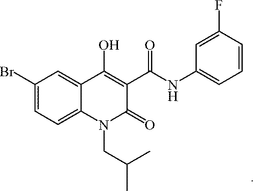

In embodiments, the compound has the formula:

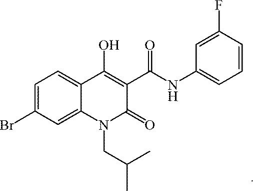

In embodiments, the compound has the formula:

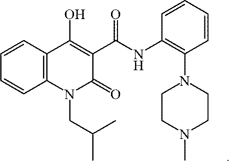

In embodiments, the compound has the formula:

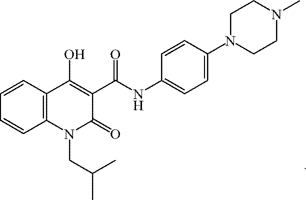

In embodiments, the compound has the formula:

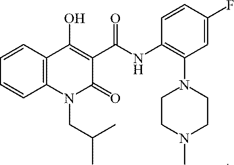

In embodiments, the compound has the formula:

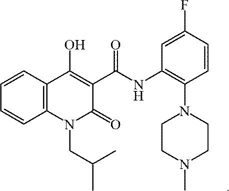

In embodiments, the compound has the formula:

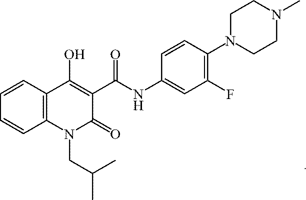

In embodiments, the compound has the formula:

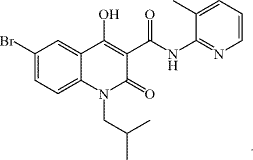

In embodiments, the compound has the formula:

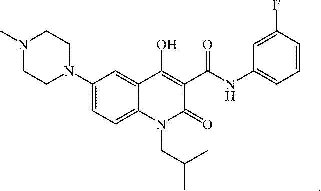

In embodiments, the compound has the formula:

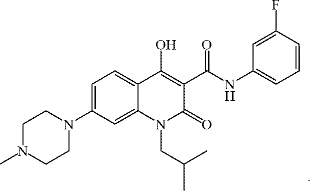

In embodiments, the compound has the formula:

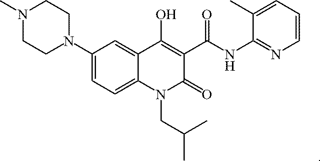

In embodiments, the compound has the formula:

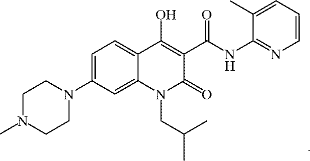

In embodiments, the compound has the formula:

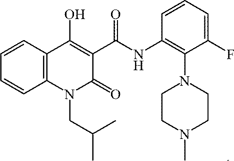

In embodiments, the compound has the formula:

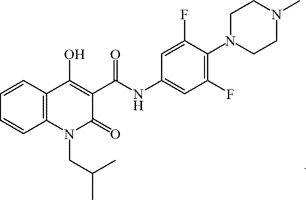

In embodiments, the compound has the formula:

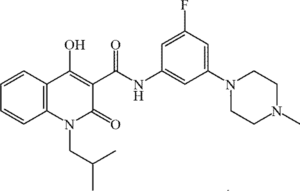

In embodiments, the compound has the formula:

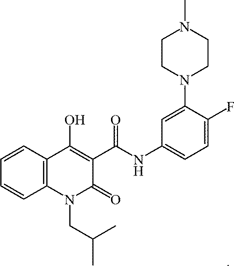

In embodiments, the compound has the formula:

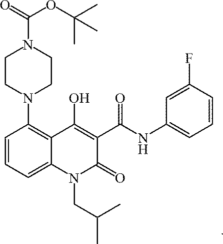

In embodiments, the compound has the formula:

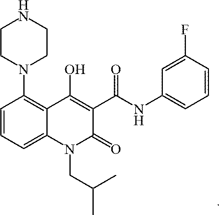

In embodiments, the compound has the formula:

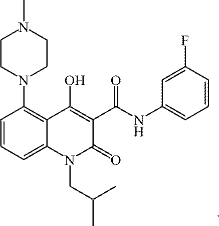

In embodiments, the compound has the formula:

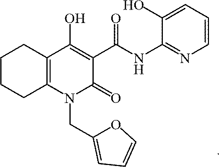

In embodiments, the compound has the formula:

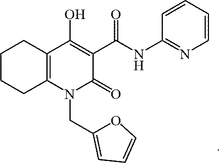

In embodiments, the compound has the formula:

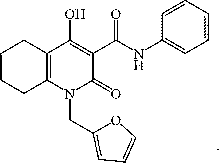

In embodiments, the compound has the formula:

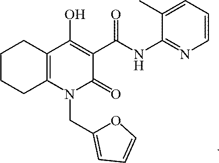

In embodiments, the compound has the formula:

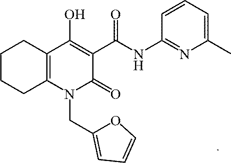

In embodiments, the compound has the formula:

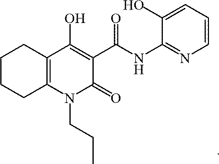

In embodiments, the compound has the formula:

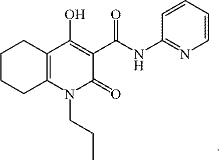

In embodiments, the compound has the formula:

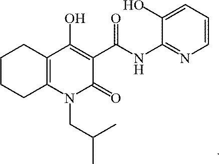

In embodiments, the compound has the formula:

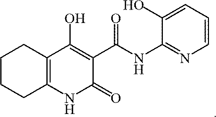

In embodiments, the compound has the formula:

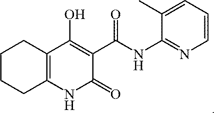

In embodiments, the compound has the formula:

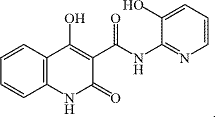

In embodiments, the compound has the formula:

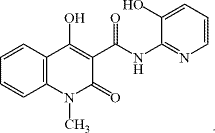

In embodiments, the compound has the formula:

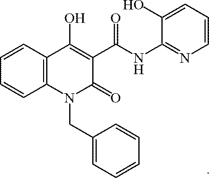

In embodiments, the compound has the formula:

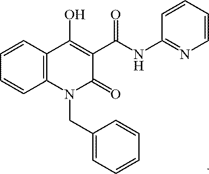

In embodiments, the compound has the formula:

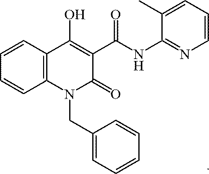

In embodiments, the compound has the formula:

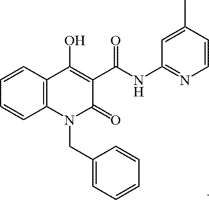

In embodiments, the compound has the formula:

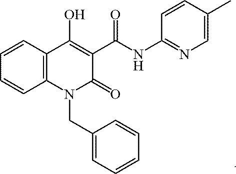

In embodiments, the compound has the formula:

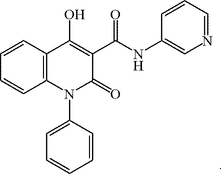

In embodiments, the compound has the formula:

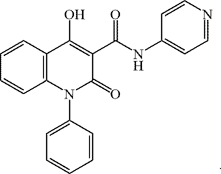

In embodiments, the compound has the formula:

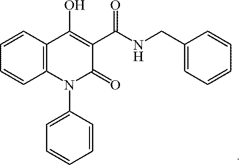

In embodiments, the compound has the formula:

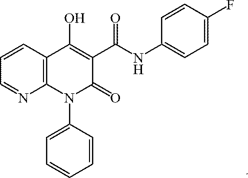

In embodiments, the compound has the formula:

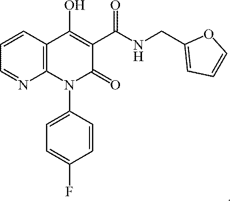

In embodiments, the compound has the formula:

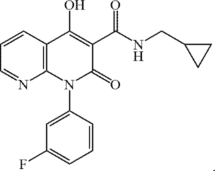

In embodiments, the compound has the formula:

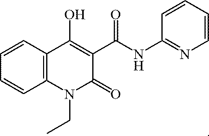

In embodiments, the compound has the formula:

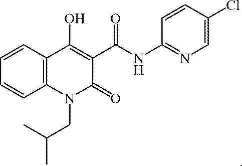

In embodiments, the compound has the formula:

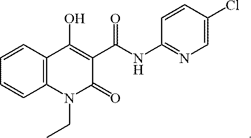

In embodiments, the compound has the formula:

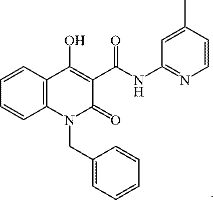

In embodiments, the compound has the formula:

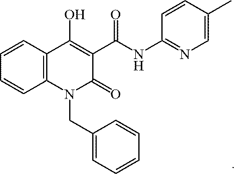

In embodiments, the compound has the formula:

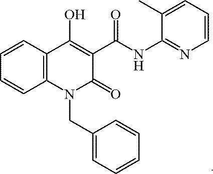

In embodiments, the compound has the formula:

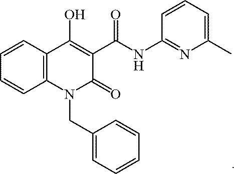

In embodiments, the compound has the formula:

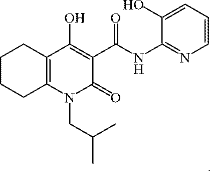

In embodiments, the compound has the formula:

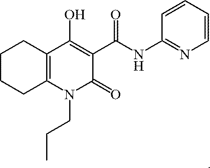

In embodiments, the compound has the formula:

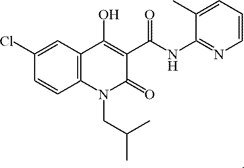

In embodiments, the compound has the formula:

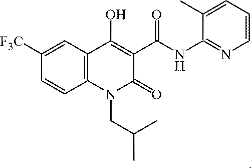

In embodiments, the compound has the formula:

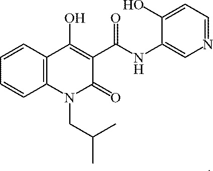

In embodiments, the compound has the formula:

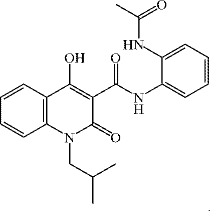

In embodiments, the compound has the formula:

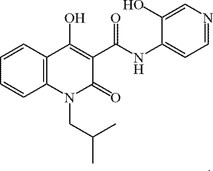

In embodiments, the compound has the formula:

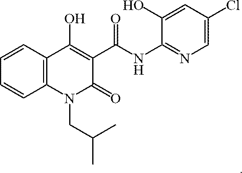

In embodiments, the compound has the formula:

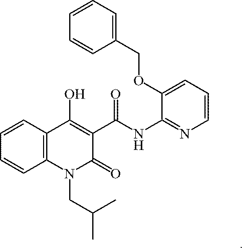

In embodiments, the compound has the formula:

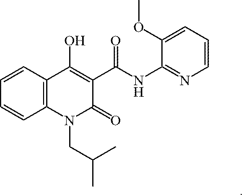

In embodiments, the compound has the formula:

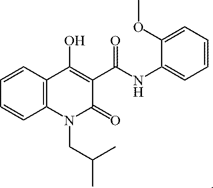

In embodiments, the compound has the formula:

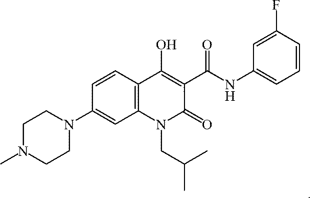

In embodiments, the salt (e.g., pharmaceutically acceptable salt) of the compound described above is the HCl salt. In embodiments, the salt (e.g., pharmaceutically acceptable salt) of the compound described above is the Cl⁻ salt. In embodiments, the salt (e.g., pharmaceutically acceptable salt) of the compound described above is the F₃CC(O)OH salt. In embodiments, the salt (e.g., pharmaceutically acceptable salt) of the compound described above is the F₃CC(O)O⁻ salt. In embodiments, the salt (e.g., pharmaceutically acceptable salt) of the compound described above is the HC(O)OH salt. In embodiments, the salt (e.g., pharmaceutically acceptable salt) of the compound described above is the HC(O)O⁻ salt. In embodiments, the salt of the compound described above is a pharmaceutically acceptable salt.

In embodiments, when R¹ is substituted, R¹ is substituted with one or more first substituent groups denoted by R^{1.1} as explained in the definitions section above in the description of “first substituent group(s)”. In embodiments, when an R^{1.1} substituent group is substituted, the R^{1.1} substituent group is substituted with one or more second substituent groups denoted by R^{1.2} as explained in the definitions section above in the description of “first substituent group(s)”. In embodiments, when an R^{1.2} substituent group is substituted, the R^{1.2} substituent group is substituted with one or more third substituent groups denoted by R^{1.3} as explained in the definitions section above in the description of “first substituent group(s)”. In the above embodiments, R¹, R^{1.1}, R^{1.2}, and R^{1.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R¹, R^{1.1}, R^{1.2}, and R^{1.3}, respectively.

In embodiments, when R^{1A} is substituted, R^{1A} is substituted with one or more first substituent groups denoted by R^{1A.1} as explained in the definitions section above in the description of “first substituent group(s)”. In embodiments, when an R^{1A.1} substituent group is substituted, the R^{1A1} substituent group is substituted with one or more second substituent groups denoted by R^{1A}^{.}² as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1} , R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{1A}, R^{1A.1}, R^{1A.2}, and R^{1A.3}, respectively.

In embodiments, when R^{1B} is substituted, R^{1B} is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B.2} as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2} , and R^{1B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{1B}, R^{1B.1}, R^{1B.2} , and R^{1B.3}, respectively.

In embodiments, when R^{1A} and R^{1B} substituents that are bonded to the same nitrogen atom are joined to form a moiety that is substituted (e.g., a substituted heterocycloalkyl or substituted heteroaryl), the moiety is substituted with one or more first substituent groups denoted by R^{1A.1} as explained in the definitions section above in the description of “first substituent group(s)”. In embodiments, when an R^{1A.1} substituent group is substituted, the R^{1A.1} substituent group is substituted with one or more second substituent groups denoted by R^{1A}^{.}² as explained in the definitions section above in the description of “first substituent group(s)”. In embodiments, when an R^{1A.2} substituent group is substituted, the R^{1A.2} substituent group is substituted with one or more third substituent groups denoted by R^{1A.3} as explained in the definitions section above in the description of “first substituent group(s)”. In the above embodiments, R^{1A}, R^{1A.1}, R^{1A.2}, and R^{1A.3} have values corresponding to the values of R^{WW}, R^{WW.1}, R:^{WW.2} , and R^{WW.2}, respectively, as explained in the definitions section above in the description of “first substituent group(s)”, wherein R^{WW}, R^{WW.1}, R^{WW.2}, and R^{WW.2} correspond to R^{1A}, R^{1A.1}, R^{1A.2}, and R^{1A.3}, respectively.

In embodiments, when R^{1A} and R^{1B} substituents that are bonded to the same nitrogen atom are joined to form a moiety that is substituted (e.g., a substituted heterocycloalkyl or substituted heteroaryl), the moiety is substituted with one or more first substituent groups denoted by R^{1B.1} as explained in the definitions section above in the description of “first substituent group(s)”. In embodiments, when an R^{1B.1} substituent group is substituted, the R^{1B.1} substituent group is substituted with one or more second substituent groups denoted by R^{1B}^{.}² as explained in the definitions section above in the description of “first substituent group(s)”. In embodiments, when an R^{1B.2} substituent group is substituted, the R^{1B.2} substituent group is substituted with one or more third substituent groups denoted by R^{1B.3} as explained in the definitions section above in the description of “first substituent group(s)”. In the above embodiments, R^{1B}, R^{1B.1}, R^{1B.2} , and R^{1B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{1B}, R^{1B.1}, R^{1B.2} , and R^{1B.3}, respectively.

In embodiments, when R^{1C} is substituted, R^{1C} is substituted with one or more first substituent groups denoted by R^{1C.1} as explained in the definitions section above in the description of “first substituent group(s)”. In embodiments, when an R^{1C.1} substituent group is substituted, the R^{1C.1} substituent group is substituted with one or more second substituent groups denoted by R^{1C.2} as explained in the definitions section above in the description of “first substituent group(s)”. In embodiments, when an R^{1C.2} substituent group is substituted, the R^{1C.2} substituent group is substituted with one or more third substituent groups denoted by R^{1C.3} as explained in the definitions section above in the description of “first substituent group(s)”. In the above embodiments, R^{1C}, R^{1C.1}, R^{1C.2}, and R^{1C.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{1C}, R^{1C.1}, R^{1C.2}, and R^{1C.3}, respectively.

In embodiments, when R^{1D} is substituted, R^{1D} is substituted with one or more first substituent groups denoted by R^{1D.1} as explained in the definitions section above in the description of “first substituent group(s)”. In embodiments, when an R^{1D.1} substituent group is substituted, the R^{1D.1} substituent group is substituted with one or more second substituent groups denoted by R^{1D.2} as explained in the definitions section above in the description of “first substituent group(s)”. In embodiments, when an R^{1D.2} substituent group is substituted, the R^{1D.2} substituent group is substituted with one or more third substituent groups denoted by R^{1D.3} as explained in the definitions section above in the description of “first substituent group(s)”. In the above embodiments, R^{1D}, R^{1D.1}, R^{1D.2}, and R^{1D}^{.}³ have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{1D}, R^{1D.1}, R^{1D.2}, and R^{1D.3}, respectively.

In embodiments, when R² is substituted, R² is substituted with one or more first substituent groups denoted by R^{2.1} as explained in the definitions section above in the description of “first substituent group(s)”. In embodiments, when an R^{2.1} substituent group is substituted, the R^{2.1} substituent group is substituted with one or more second substituent groups denoted by R^{2.2} as explained in the definitions section above in the description of “first substituent group(s)”. In embodiments, when an R^{2.2} substituent group is substituted, the R^{2.2} substituent group is substituted with one or more third substituent groups denoted by R^{2.3} as explained in the definitions section above in the description of “first substituent group(s)”. In the above embodiments, R², R^{2.1}, R^{2.2}, and R^{2.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R², R^{2.1}, R^{2.2}, and R^{2.3}, respectively.

In embodiments, when R^{2A} is substituted, R^{2A} is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{2A}, R^{2A.1}, R^{2A.2}, and R^{2A.3}, respectively.

In embodiments, when R^{2B} is substituted, R^{2B} is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{2B}, R^{2B.1}, R^{2B.2}, and R^{2B.3}, respectively.

In embodiments, when R^{2A} and R^{2B} substituents that are bonded to the same nitrogen atom are joined to form a moiety that is substituted (e.g., a substituted heterocycloalkyl or substituted heteroaryl), the moiety is substituted with one or more first substituent groups denoted by R^{2A.1} as explained in the definitions section above in the description of “first substituent group(s)”. In embodiments, when an R^{2A.1} substituent group is substituted, the R^{2A.1} substituent group is substituted with one or more second substituent groups denoted by R^{2A.2} as explained in the definitions section above in the description of “first substituent group(s)”. In embodiments, when an R^{2A.2} substituent group is substituted, the R^{2A.2} substituent group is substituted with one or more third substituent groups denoted by R^{2A.3} as explained in the definitions section above in the description of “first substituent group(s)”. In the above embodiments, R^{2A}, R^{2A.1}, R^{2A.2}, and R^{2A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{2A}, R^{2A.1}, R^{2A.2}, and R^{2A.3}, respectively.

In embodiments, when R^{2A} and R^{2B} substituents that are bonded to the same nitrogen atom are joined to form a moiety that is substituted (e.g., a substituted heterocycloalkyl or substituted heteroaryl), the moiety is substituted with one or more first substituent groups denoted by R^{2B.1} as explained in the definitions section above in the description of “first substituent group(s)”. In embodiments, when an R^{2B.1} substituent group is substituted, the R^{2B.1} substituent group is substituted with one or more second substituent groups denoted by R^{2B.2} as explained in the definitions section above in the description of “first substituent group(s)”. In embodiments, when an R^{2B.2} substituent group is substituted, the R^{2B.2} substituent group is substituted with one or more third substituent groups denoted by R^{2B.3} as explained in the definitions section above in the description of “first substituent group(s)”. In the above embodiments, R^{2B}, R^{2B.1}, R^{2B.2}, and R^{2B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{2B}, R^{2B.1}, R^{2B.2}, and R^{2B.3}, respectively.

In embodiments, when R^{2C} is substituted, R^{2C} is substituted with one or more first substituent groups denoted by R^{2C.1} as explained in the definitions section above in the description of “first substituent group(s)”. In embodiments, when an R^{2C.1} substituent group is substituted, the R^{2C.1} substituent group is substituted with one or more second substituent groups denoted by R^{2C.2} as explained in the definitions section above in the description of “first substituent group(s)”. In embodiments, when an R^{2C.2} substituent group is substituted, the R^{2C.2} substituent group is substituted with one or more third substituent groups denoted by R^{2C.3} as explained in the definitions section above in the description of “first substituent group(s)”. In the above embodiments, R^{2C}, R^{2C.1}, R^{2C.2}, and R^{2C.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{2C}, R^{2C.1}, R2^{2C.2} and R^{2C.3} , respectively.

In embodiments, when R^{2D} is substituted, R^{2D} is substituted with one or more first substituent groups denoted by R^{2D.1} as explained in the definitions section above in the description of “first substituent group(s)”. In embodiments, when an R^{2D.1} substituent group is substituted, the R^{2D.1} substituent group is substituted with one or more second substituent groups denoted by R^{2D.2} as explained in the definitions section above in the description of “first substituent group(s)”. In embodiments, when an R^{2D.2} substituent group is substituted, the R^{2D.2} substituent group is substituted with one or more third substituent groups denoted by R^{2D.3} as explained in the definitions section above in the description of “first substituent group(s)”. In the above embodiments, R^{2D}, R^{2D.1}, R^{2D.2}, and R^{2D.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{2D}, R^{2D.1}, R^{2D.2}, and R^{2D.3} , respectively.

In embodiments, when R³ is substituted, R³ is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R³, R^{3.1}, R^{3.2}, and R^{3.3}, respectively.

In embodiments, when two adjacent R³ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{3.1} as explained in the definitions section above in the description of “first substituent group(s)”. In embodiments, when an R^{3.1} substituent group is substituted, the R^{3.1} substituent group is substituted with one or more second substituent groups denoted by R^{3.2} as explained in the definitions section above in the description of “first substituent group(s)”. In embodiments, when an R^{3.2} substituent group is substituted, the R^{3.2} substituent group is substituted with one or more third substituent groups denoted by R^{3.3} as explained in the definitions section above in the description of “first substituent group(s)”. In the above embodiments, R³, R^{3.1}, R^{3.2}, and R^{3.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R³, R^{3.1}, R^{3.2}, and R^{3.3}, respectively.

In embodiments, when R^{3A} is substituted, R^{3A} is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{3A}, R^{3A.1}, R^{3A.2}, and R^{3A.3}, respectively.

In embodiments, when R^{3B} is substituted, R^{3B} is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW}^{.3} correspond to R^{3B}, R^{3B.1}, R^{3B.2}, and R^{3B.3}, respectively.

In embodiments, when R^{3A} and R^{3B} substituents that are bonded to the same nitrogen atom are joined to form a moiety that is substituted (e.g., a substituted heterocycloalkyl or substituted heteroaryl), the moiety is substituted with one or more first substituent groups denoted by R^{3A.1} as explained in the definitions section above in the description of “first substituent group(s)”. In embodiments, when an R^{3A.1} substituent group is substituted, the R^{3A.1} substituent group is substituted with one or more second substituent groups denoted by R^{3A.2} as explained in the definitions section above in the description of “first substituent group(s)”. In embodiments, when an R^{3A.2} substituent group is substituted, the R^{3A.2} substituent group is substituted with one or more third substituent groups denoted by R^{3A.3} as explained in the definitions section above in the description of “first substituent group(s)”. In the above embodiments, R^{3A}, R^{3A.1}, R^{3A.2}, and R^{3A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{3A}, R^{3A1}, R^{3A.2}, and R^{3A.3}, respectively.

In embodiments, when R^{3A} and R^{3B} substituents that are bonded to the same nitrogen atom are joined to form a moiety that is substituted (e.g., a substituted heterocycloalkyl or substituted heteroaryl), the moiety is substituted with one or more first substituent groups denoted by R^{3B.1} as explained in the definitions section above in the description of “first substituent group(s)”. In embodiments, when an R^{3B.1} substituent group is substituted, the R^{3B.1} substituent group is substituted with one or more second substituent groups denoted by R^{3B.2} as explained in the definitions section above in the description of “first substituent group(s)”. In embodiments, when an R^{3B.2} substituent group is substituted, the R^{3B.2} substituent group is substituted with one or more third substituent groups denoted by R^{3B.3} as explained in the definitions section above in the description of “first substituent group(s)”. In the above embodiments, R^{3B}, R^{3B.1}, R^{3B.2}, and R^{3B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{3B}, R^{3B.1}, R^{3B.2}, and R^{3B.3}, respectively.

In embodiments, when R^{3C} is substituted, R^{3C} is substituted with one or more first substituent groups denoted by R^{3C.1} as explained in the definitions section above in the description of “first substituent group(s)”. In embodiments, when an R^{3C.1} substituent group is substituted, the R^{3C.1} substituent group is substituted with one or more second substituent groups denoted by R^{3C.2} as explained in the definitions section above in the description of “first substituent group(s)”. In embodiments, when an R^{3C.2} substituent group is substituted, the R^{3C.2} substituent group is substituted with one or more third substituent groups denoted by R^{3C.3} as explained in the definitions section above in the description of “first substituent group(s)”. In the above embodiments, R^{3C}, R^{3C.1}, R^{3C.2}, and R^{3C.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{3C}, R^{3C.1}, R^{3C.2}, and R^{3C.3}, respectively.

In embodiments, when R^{3D} is substituted, R^{3D} is substituted with one or more first substituent groups denoted by R^{3D.1} as explained in the definitions section above in the description of “first substituent group(s)”. In embodiments, when an R^{3D.1} substituent group is substituted, the R^{3D.1} substituent group is substituted with one or more second substituent groups denoted by R^{3D.2} as explained in the definitions section above in the description of “first substituent group(s)”. In embodiments, when an R^{3D.2} substituent group is substituted, the R^{3D.2} substituent group is substituted with one or more third substituent groups denoted by R^{3D.3} as explained in the definitions section above in the description of “first substituent group(s)”. In the above embodiments, R^{3D}, R^{3D.1}, R^{3D.2}, and R^{3D.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{3D}, R^{3D.1}, R^{3D.2}, and R^{3D.3}, respectively.

In embodiments, when R⁴ is substituted, R⁴ is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R⁴, R^{4.1}, R^{4.2}, and R^{4.3}, respectively.

In embodiments, when two adjacent R⁴ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{4.1} as explained in the definitions section above in the description of “first substituent group(s)”. In embodiments, when an R^{4.1} substituent group is substituted, the R^{4.1} substituent group is substituted with one or more second substituent groups denoted by R^{4.2} as explained in the definitions section above in the description of “first substituent group(s)”. In embodiments, when an R^{4.2} substituent group is substituted, the R^{4.2} substituent group is substituted with one or more third substituent groups denoted by R^{4.3} as explained in the definitions section above in the description of “first substituent group(s)”. In the above embodiments, R⁴, R^{4.1}, R^{4.2}, and R^{4.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R⁴, R^{4.1}, R^{4.2}, and R^{4.3}, respectively.

In embodiments, when R^{4A} is substituted, R^{4A} is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{4A}, R^{4A.1}, R^{4A.2}, and R^{4A.3}, respectively.

In embodiments, when R^{4B} is substituted, R^{4B} is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{4B}, R^{4B.1}, R^{4B.2}, and R^{4B.3}, respectively.

In embodiments, when R^{4A} and R^{4B} substituents that are bonded to the same nitrogen atom are joined to form a moiety that is substituted (e.g., a substituted heterocycloalkyl or substituted heteroaryl), the moiety is substituted with one or more first substituent groups denoted by R^{4A.1} as explained in the definitions section above in the description of “first substituent group(s)”. In embodiments, when an R^{4A.1} substituent group is substituted, the R^{4A.1} substituent group is substituted with one or more second substituent groups denoted by R^{4A.2} as explained in the definitions section above in the description of “first substituent group(s)”. In embodiments, when an R^{4A.2} substituent group is substituted, the R^{4A.2} substituent group is substituted with one or more third substituent groups denoted by R^{4A.3} as explained in the definitions section above in the description of “first substituent group(s)”. In the above embodiments, R^{4A}, R^{4A.1}, R^{4A.2}, and R^{4A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{4A}, R^{4A.1}, R^{4A.2}, and R^{4A.3}, respectively.

In embodiments, when R^{4A} and R^{4B} substituents that are bonded to the same nitrogen atom are joined to form a moiety that is substituted (e.g., a substituted heterocycloalkyl or substituted heteroaryl), the moiety is substituted with one or more first substituent groups denoted by R^{4B.1} as explained in the definitions section above in the description of “first substituent group(s)”. In embodiments, when an R^{4B.1} substituent group is substituted, the R^{4B.1} substituent group is substituted with one or more second substituent groups denoted by R^{4B.2} as explained in the definitions section above in the description of “first substituent group(s)”. In embodiments, when an R^{4B.2} substituent group is substituted, the R^{4B.2} substituent group is substituted with one or more third substituent groups denoted by R^{4B.3} as explained in the definitions section above in the description of “first substituent group(s)”. In the above embodiments, R^{4B}, R^{4B.1}, R^{4B.2}, and R^{4B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{4B}, R^{4B.1}, R^{4B.2}, and R^{4B.3}, respectively.

In embodiments, when R^{4C} is substituted, R^{4C} is substituted with one or more first substituent groups denoted by R^{4C.1} as explained in the definitions section above in the description of “first substituent group(s)”. In embodiments, when an R^{4C.1} substituent group is substituted, the R^{4C.1} substituent group is substituted with one or more second substituent groups denoted by R^{4C.2} as explained in the definitions section above in the description of “first substituent group(s)”. In embodiments, when an R^{4C.2} substituent group is substituted, the R^{4C.2} substituent group is substituted with one or more third substituent groups denoted by R^{4C.3} as explained in the definitions section above in the description of “first substituent group(s)”. In the above embodiments, R^{4C}, R^{4C.1}, R^{4C.2}, and R^{4C.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{4C}, R^{4C.1}, R^{4C.2}, and R^{4C.3}, respectively.

In embodiments, when R^{4D} is substituted, R^{4D} is substituted with one or more first substituent groups denoted by R^{4D.1} as explained in the definitions section above in the description of “first substituent group(s)”. In embodiments, when an R^{4D.1} substituent group is substituted, the R^{4D.1} substituent group is substituted with one or more second substituent groups denoted by R^{4D.2} as explained in the definitions section above in the description of “first substituent group(s)”. In embodiments, when an R^{4D.2} substituent group is substituted, the R^{4D.2} substituent group is substituted with one or more third substituent groups denoted by R^{4D.3} as explained in the definitions section above in the description of “first substituent group(s)”. In the above embodiments, R^{4D}, R^{4D.1}, R^{4D.2}, and R^{4D.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{4D}, R^{4D.1}, R^{4D.2}, and R^{4D.3}, respectively.

In embodiments, when R¹⁰ is substituted, R¹⁰ is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.1}, R^{10.2}, and R^{10.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R¹⁰, R^{10.1} R^{10.2}, and R^{10.3}, respectively.

In embodiments, when two adjacent R¹⁰ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{10.1} as explained in the definitions section above in the description of “first substituent group(s)”. In embodiments, when an R^{10.1} substituent group is substituted, the R^{10.1} substituent group is substituted with one or more second substituent groups denoted by R^{10.2} as explained in the definitions section above in the description of “first substituent group(s)”. In embodiments, when an R^{10.2} substituent group is substituted, the R^{10.2} substituent group is substituted with one or more third substituent groups denoted by R^{10.3} as explained in the definitions section above in the description of “first substituent group(s)”. In the above embodiments, R¹⁰, R^{10.1}, R^{10.2}, and R^{10.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R¹⁰, R^{10.1} R^{10.2}, and R^{10.3}, respectively.

In embodiments, when R^{10A} is substituted, R^{10A} is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A}, R^{10A.1}, R^{10A.2}, and R^{10A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10A}, R^{10A.1}, R^{10A.2}, and R^{10A.3}, respectively.

In embodiments, when R^{10B} is substituted, R^{10B} is substituted with one or more first substituent groups denoted by R^{10B.1} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B}, R^{10B.1}, R^{10B.2}, and R^{10B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10B}, R^{10B.1}, R^{10B.2}, and R^{10B.3}, respectively.

In embodiments, when R^{10A} and R^{10B} substituents that are bonded to the same nitrogen atom are joined to form a moiety that is substituted (e.g., a substituted heterocycloalkyl or substituted heteroaryl), the moiety is substituted with one or more first substituent groups denoted by R^{10A.1} as explained in the definitions section above in the description of “first substituent group(s)”. In embodiments, when an R^{10A.1} substituent group is substituted, the R^{10A.1} substituent group is substituted with one or more second substituent groups denoted by R^{10A.2} as explained in the definitions section above in the description of “first substituent group(s)”. In embodiments, when an R^{10A.2} substituent group is substituted, the R^{10A.2} substituent group is substituted with one or more third substituent groups denoted by R^{10A.3} as explained in the definitions section above in the description of “first substituent group(s)”. In the above embodiments, R^{10A}, R^{10A.1}, R^{10A.2}, and R^{10A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10A}, R^{10A.1}, R^{10A.2}, and R^{10A.3}, respectively.

In embodiments, when R^{10A} and R^{10B} substituents that are bonded to the same nitrogen atom are joined to form a moiety that is substituted (e.g., a substituted heterocycloalkyl or substituted heteroaryl), the moiety is substituted with one or more first substituent groups denoted by R^{10B.1} as explained in the definitions section above in the description of “first substituent group(s)”. In embodiments, when an R^{10B.1} substituent group is substituted, the R^{10B.1} substituent group is substituted with one or more second substituent groups denoted by R^{10B.2} as explained in the definitions section above in the description of “first substituent group(s)”. In embodiments, when an R^{10B.2} substituent group is substituted, the R^{10B.2} substituent group is substituted with one or more third substituent groups denoted by R^{10B.3} as explained in the definitions section above in the description of “first substituent group(s)”. In the above embodiments, R^{10B}, R^{10B.1}, R^{10B.2}, and R^{10B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10B}, R^{10B.1}, R^{10B.2}, and R^{10B.3}, respectively.

In embodiments, when R^{10C} is substituted, R^{10C} is substituted with one or more first substituent groups denoted by R^{10C.1} as explained in the definitions section above in the description of “first substituent group(s)”. In embodiments, when an R^{10C.1} substituent group is substituted, the R^{10C.1} substituent group is substituted with one or more second substituent groups denoted by R^{10C.2} as explained in the definitions section above in the description of “first substituent group(s)”. In embodiments, when an R^{10C.2} substituent group is substituted, the R^{10C.2} substituent group is substituted with one or more third substituent groups denoted by R^{10C.3} as explained in the definitions section above in the description of “first substituent group(s)”. In the above embodiments, R^{10C}, R^{10C.1}, R^{10C.2}, and R^{10C.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10C}, R^{10C.1}, R^{10C.2}, and R^{10C.3}, respectively.

In embodiments, when R^{10D} is substituted, R^{10D} is substituted with one or more first substituent groups denoted by R^{10D.1} as explained in the definitions section above in the description of “first substituent group(s)”. In embodiments, when an R^{10D.1} substituent group is substituted, the R^{10D.1} substituent group is substituted with one or more second substituent groups denoted by R^{10D.2} as explained in the definitions section above in the description of “first substituent group(s)”. In embodiments, when an R^{10D.2} substituent group is substituted, the R^{10D.2} substituent group is substituted with one or more third substituent groups denoted by R^{10D.3} as explained in the definitions section above in the description of “first substituent group(s)”. In the above embodiments, R^{10D}, R^{10D.1}, R^{10D.2}, and R^{10D.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10D}, R^{10D.1}, R^{10D.2}, and R^{10D.3}, respectively.

In embodiments, when R^{10.A} is substituted, R^{10.A} is substituted with one or more first substituent groups denoted by R¹⁰^{.}^{A.1} as explained in the definitions section above in the description of “first substituent group(s)”. In embodiments, when an R^{10.A.1} substituent group is substituted, the R^{10.A.1} substituent group is substituted with one or more second substituent groups denoted by R^{10.A.2} as explained in the definitions section above in the description of “first substituent group(s)”. In embodiments, when an R^{10.A.2} substituent group is substituted, the R^{10.A.2} substituent group is substituted with one or more third substituent groups denoted by R^{10.A.3} as explained in the definitions section above in the description of “first substituent group(s)”. In the above embodiments, R^{10.A}, R¹⁰^{.}^{A.1}, R^{10.A.2}, and R¹⁰^{.}^{A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10.A}, R^{10.A.1}, R^{10.A.2}, and R^{10.A.3}, respectively.

In embodiments, when R^{10.B} is substituted, R^{10.B} is substituted with one or more first substituent groups denoted by R^{10.B.1} as explained in the definitions section above in the description of “first substituent group(s)”. In embodiments, when an R^{10.B.1} substituent group is substituted, the R^{10.B.1} substituent group is substituted with one or more second substituent groups denoted by R^{10.B.2} as explained in the definitions section above in the description of “first substituent group(s)”. In embodiments, when an R^{10.B.2} substituent group is substituted, the R^{10.B.2} substituent group is substituted with one or more third substituent groups denoted by R^{10.B.3} as explained in the definitions section above in the description of “first substituent group(s)”. In the above embodiments, R¹⁰^{.}^{B}, R¹⁰^{.}^{B.1}, R¹⁰^{.}^{B-2}, and R¹⁰^{.}^{B.3} have values corresponding to the values of R^{WW}, R^{WW.1}, R^{WW.2} and R^{WW.2}, respectively, as explained in the definitions section above in the description of “first substituent group(s)”, wherein R^{WW}, R^{WW.1}, R^{WW.2}, and R^{WW.2} correspond to R^{10.B}, R^{10.B.1}, R^{10.B.2}, and R^{10.B.3}, respectively.

In embodiments, when R^{10.C} is substituted, R^{10.C} is substituted with one or more first substituent groups denoted by R^{10.C.1} as explained in the definitions section above in the description of “first substituent group(s)”. In embodiments, when an R^{10.C.1} substituent group is substituted, the R^{10.C.1} substituent group is substituted with one or more second substituent groups denoted by R^{10.C.2} as explained in the definitions section above in the description of “first substituent group(s)”. In embodiments, when an R^{10.C.2} substituent group is substituted, the R^{10.C.2} substituent group is substituted with one or more third substituent groups denoted by R^{10.C.3} as explained in the definitions section above in the description of “first substituent group(s)”. In the above embodiments, R^{10.C}, R^{10.C.1}, R^{10.C.2}, and R^{10.C.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10.C}, R^{10.C.1}, R^{10.C.2}, and R^{10.C.3}, respectively.

In embodiments, when R^{10.D} is substituted, R^{10.D} is substituted with one or more first substituent groups denoted by R^{10.D.1} as explained in the definitions section above in the description of “first substituent group(s)”. In embodiments, when an R^{10.D.1} substituent group is substituted, the R^{10.D.1} substituent group is substituted with one or more second substituent groups denoted by R^{10.D.2} as explained in the definitions section above in the description of “first substituent group(s)”. In embodiments, when an R^{10.D.2} substituent group is substituted, the R^{10.D.2} substituent group is substituted with one or more third substituent groups denoted by R^{10.D.3} as explained in the definitions section above in the description of “first substituent group(s)”. In the above embodiments, R^{10.D}, R¹⁰^{.}^{D.1}, R¹⁰^{.}^{D.2}, and R¹⁰^{.}^{D.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10.D}, R^{10.D.1}, R^{10.D.2}, and R^{10.D.3}, respectively.

In embodiments, when R^{10.E} is substituted, R^{10.E} is substituted with one or more first substituent groups denoted by R^{10.E.1} as explained in the definitions section above in the description of “first substituent group(s)”. In embodiments, when an R^{10.E.1} substituent group is substituted, the R^{10.E.1} substituent group is substituted with one or more second substituent groups denoted by R^{10.E.2} as explained in the definitions section above in the description of “first substituent group(s)”. In embodiments, when an R^{10.E.2} substituent group is substituted, the R^{10.E.2} substituent group is substituted with one or more third substituent groups denoted by R^{10.E.3} as explained in the definitions section above in the description of “first substituent group(s)”. In the above embodiments, R¹⁰^{.}^{E}, R¹⁰^{.}^{E.1}, R¹⁰^{.}^{E.2}, and R¹⁰^{.}^{E.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{10.E}, R^{10.E.1}, R^{10.E.2}, and R¹⁰^{.}^{E.3}, respectively.

In embodiments, when R²⁰ is substituted, R²⁰ is substituted with one or more first substituent groups denoted by R^{20.1} as explained in the definitions section above in the description of “first substituent group(s)”. In embodiments, when an R^{20.1} substituent group is substituted, the R^{20.1} substituent group is substituted with one or more second substituent groups denoted by R^{20.2} as explained in the definitions section above in the description of “first substituent group(s)”. In embodiments, when an R^{20.2} substituent group is substituted, the R^{20.2} substituent group is substituted with one or more third substituent groups denoted by R^{20.3} as explained in the definitions section above in the description of “first substituent group(s)”. In the above embodiments, R²⁰, R^{20.1}, R^{20.2}, and R^{20.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R²⁰, R^{20.1}, R^{20.2}, and R^{20.3}, respectively.

In embodiments, when two adjacent R²⁰ substituents are optionally joined to form a moiety that is substituted (e.g., a substituted cycloalkyl, substituted heterocycloalkyl, substituted aryl, or substituted heteroaryl), the moiety is substituted with one or more first substituent groups denoted by R^{20.1} as explained in the definitions section above in the description of “first substituent group(s)”. In embodiments, when an R^{20.1} substituent group is substituted, the R^{20.1} substituent group is substituted with one or more second substituent groups denoted by R^{20.2} as explained in the definitions section above in the description of “first substituent group(s)”. In embodiments, when an R^{20.2} substituent group is substituted, the R^{20.2} substituent group is substituted with one or more third substituent groups denoted by R^{20.3} as explained in the definitions section above in the description of “first substituent group(s)”. In the above embodiments, R²⁰, R^{20.1}, R^{20.2}, and R^{20.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R²⁰, R^{20.1}, R^{20.2}, and R^{20.3}, respectively.

In embodiments, when R^{20A} is substituted, R^{20A} is substituted with one or more first substituent groups denoted by R^{20A.1} as explained in the definitions section above in the description of “first substituent group(s)”. In embodiments, when an R^{20A.1} substituent group is substituted, the R^{20A.1} substituent group is substituted with one or more second substituent groups denoted by R^{20A.2} as explained in the definitions section above in the description of “first substituent group(s)”. In embodiments, when an R^{20A.2} substituent group is substituted, the R^{20A.2} substituent group is substituted with one or more third substituent groups denoted by R^{20A.3} as explained in the definitions section above in the description of “first substituent group(s)”. In the above embodiments, R^{20A}, R^{20A.1}, R^{20A.2}, and R^{20A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{20A}, R^{20A.1}, R^{20A.2}, and R^{20A.3}, respectively.

In embodiments, when R^{20B} is substituted, R^{20B} is substituted with one or more first substituent groups denoted by R^{20B.1} as explained in the definitions section above in the description of “first substituent group(s)”. In embodiments, when an R^{20B.1} substituent group is substituted, the R^{20B.1} substituent group is substituted with one or more second substituent groups denoted by R^{20B.2} as explained in the definitions section above in the description of “first substituent group(s)”. In embodiments, when an R^{20B.2} substituent group is substituted, the R^{20B.2} substituent group is substituted with one or more third substituent groups denoted by R^{20B.3} as explained in the definitions section above in the description of “first substituent group(s)”. In the above embodiments, R^{20B}, R^{20B.1}, R^{20B.2}, and R^{20B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW}.³ correspond to R^{20B} R^{20B.1}, R^{20B.2}, and R^{20B.3}, respectively.

In embodiments, when R^{20A} and R^{20B} substituents that are bonded to the same nitrogen atom are joined to form a moiety that is substituted (e.g., a substituted heterocycloalkyl or substituted heteroaryl), the moiety is substituted with one or more first substituent groups denoted by R^{20A.1} as explained in the definitions section above in the description of “first substituent group(s)”. In embodiments, when an R^{20A.1} substituent group is substituted, the R^{20A.1} substituent group is substituted with one or more second substituent groups denoted by R^{20A.2} as explained in the definitions section above in the description of “first substituent group(s)”. In embodiments, when an R^{20A.2} substituent group is substituted, the R^{20A.2} substituent group is substituted with one or more third substituent groups denoted by R^{20A.3} as explained in the definitions section above in the description of “first substituent group(s)”. In the above embodiments, R^{20A}, R^{20A.1}, R^{20A.2}, and R^{20A.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{20A}, R^{20A.1}, R^{20A.2}, and R^{20A.3}, respectively.

In embodiments, when R^{20A} and R^{20B} substituents that are bonded to the same nitrogen atom are joined to form a moiety that is substituted (e.g., a substituted heterocycloalkyl or substituted heteroaryl), the moiety is substituted with one or more first substituent groups denoted by R^{20B.1} as explained in the definitions section above in the description of “first substituent group(s)”. In embodiments, when an R^{20B.1} substituent group is substituted, the R^{20B.1} substituent group is substituted with one or more second substituent groups denoted by R^{20B.2} as explained in the definitions section above in the description of “first substituent group(s)”. In embodiments, when an R^{20B.2} substituent group is substituted, the R^{20B.2} substituent group is substituted with one or more third substituent groups denoted by R^{20B.3} as explained in the definitions section above in the description of “first substituent group(s)”. In the above embodiments, R^{20B}, R^{20B.1}, R^{20B.2}, and R^{20B.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{20B} R^{20B.1}, R^{20B.2}, and R^{20B.3}, respectively.

In embodiments, when R^{20C} is substituted, R^{20C} is substituted with one or more first substituent groups denoted by R^{20C.1} as explained in the definitions section above in the description of “first substituent group(s)”. In embodiments, when an R^{20C.1} substituent group is substituted, the R^{20C.1} substituent group is substituted with one or more second substituent groups denoted by R^{20C.2} as explained in the definitions section above in the description of “first substituent group(s)”. In embodiments, when an R^{20C.2} substituent group is substituted, the R^{20C.2} substituent group is substituted with one or more third substituent groups denoted by R^{20C.3} as explained in the definitions section above in the description of “first substituent group(s)”. In the above embodiments, R^{20C}, R^{20C.1}, R^{20C.2}, and R^{20C.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{20C}, R^{20C.1}, R^{20C.2}, and R^{20C.3}, respectively.

In embodiments, when R^{20D} is substituted, R^{20D} is substituted with one or more first substituent groups denoted by R^{20D.1} as explained in the definitions section above in the description of “first substituent group(s)”. In embodiments, when an R^{20D.1} substituent group is substituted, the R^{20D.1} substituent group is substituted with one or more second substituent groups denoted by R^{20D.2} as explained in the definitions section above in the description of “first substituent group(s)”. In embodiments, when an R^{20D.2} substituent group is substituted, the R^{20D.2} substituent group is substituted with one or more third substituent groups denoted by R^{20D.3} as explained in the definitions section above in the description of “first substituent group(s)”. In the above embodiments, R^{20D}, R^{20D.1}, R^{20D.2}, and R^{20D.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{20D}, R^{20D.1}, R^{20D.2}, and R^{20D.3}, respectively.

In embodiments, when L¹ is substituted, L¹ is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, L¹, R^{L1.1}, R^{L1.2}, and R^{L1.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2,} and R^{LWW.3} are L¹, R^{L1.1}, R^{L1.2}, and R^{L1.3}, respectively.

In embodiments, when R^{L1} is substituted, R^{L1} is substituted with one or more first substituent groups denoted by R^{L1.1} as explained in the definitions section above in the description of “first substituent group(s)”. In embodiments, when an R^{L1.1} substituent group is substituted, the R^{L1.1} substituent group is substituted with one or more second substituent groups denoted by R^{L1.2} as explained in the definitions section above in the description of “first substituent group(s)”. In embodiments, when an R^{L1.2} substituent group is substituted, the R^{L1.2} substituent group is substituted with one or more third substituent groups denoted by R^{L1.3} as explained in the definitions section above in the description of “first substituent group(s)”. In the above embodiments, R^{L1}, R^{L1.1}, R^{L1.2}, and R^{L1.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{L1}, R^{L1.1}, R^{L1.2}, and R^{L1.3}, respectively.

In embodiments, when L² is substituted, L² is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, L², R^{L2.1}, R^{L2.2}, and R^{L2.3} have values corresponding to the values of L^{WW}, R^{LWW.1}, R^{LWW.2}, and R^{LWW.3}, respectively, as explained in the definitions section above in the description of “first substituent group(s)”, wherein L^{WW}, R^{LWW.1}, R^{LWW.2}, and R^{LWW.3} are L², R^{L2.1}, R^{L2.2}, and R^{L2.3}, respectively.

In embodiments, when R^{L2} is substituted, R^{L2} is substituted with one or more first substituent groups denoted by R^{L2.1} as explained in the definitions section above in the description of “first substituent group(s)”. In embodiments, when an R^{L2.1} substituent group is substituted, the R^{L2.1} substituent group is substituted with one or more second substituent groups denoted by R^{L2.2} as explained in the definitions section above in the description of “first substituent group(s)”. In embodiments, when an R^{L2.2} substituent group is substituted, the R^{L2.2} substituent group is substituted with one or more third substituent groups denoted by R^{L2.3} as explained in the definitions section above in the description of “first substituent group(s)”. In the above embodiments, R^{L2}, R^{L2.1}, R^{L2.2}, and R^{L2.3} have values corresponding to the values of R^{WW}, R^{WW.1}, R^{WW.2}, and R^{WW.2}, respectively, as explained in the definitions section above in the description of “first substituent group(s)”, wherein R^{WW}, R^{WW.1}, R^{WW.2}, and R^{WW.2} correspond to R^{L2}, R^{L2.1}, R^{L2.2}, and R^{L2.3}, respectively.

In embodiments, a substituted R¹ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹ is substituted with a plurality of groups selected from substituent groups, size-limited substituent groups, and lower substituent groups; each substituent group, size-limited substituent group, and/or lower substituent group may optionally be different. In embodiments, when R¹ is substituted, it is substituted with at least one substituent group. In embodiments, when R¹ is substituted, it is substituted with at least one size-limited substituent group. In embodiments, when R¹ is substituted, it is substituted with at least one lower substituent group.

In embodiments, a substituted R^{1A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1A} is substituted with a plurality of groups selected from substituent groups, size-limited substituent groups, and lower substituent groups; each substituent group, size-limited substituent group, and/or lower substituent group may optionally be different. In embodiments, when R^{1A} is substituted, it is substituted with at least one substituent group. In embodiments, when R^{1A} is substituted, it is substituted with at least one size-limited substituent group. In embodiments, when R^{1A} is substituted, it is substituted with at least one lower substituent group.

In embodiments, a substituted R^{1B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1B} is substituted with a plurality of groups selected from substituent groups, size-limited substituent groups, and lower substituent groups; each substituent group, size-limited substituent group, and/or lower substituent group may optionally be different. In embodiments, when R^{1B} is substituted, it is substituted with at least one substituent group. In embodiments, when R^{1B} is substituted, it is substituted with at least one size-limited substituent group. In embodiments, when R^{1B} is substituted, it is substituted with at least one lower substituent group.

In embodiments, a substituted ring formed when R^{1A} and R^{1B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{1A} and R^{1B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{1A} and R^{1B} substituents bonded to the same nitrogen atom are joined is substituted, it is substituted with at least one substituent group. In embodiments, when the substituted ring formed when R^{1A} and R^{1B} substituents bonded to the same nitrogen atom are joined is substituted, it is substituted with at least one size-limited substituent group. In embodiments, when the substituted ring formed when R^{1A} and R^{1B} substituents bonded to the same nitrogen atom are joined is substituted, it is substituted with at least one lower substituent group.

In embodiments, a substituted R^{1C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1C} is substituted with a plurality of groups selected from substituent groups, size-limited substituent groups, and lower substituent groups; each substituent group, size-limited substituent group, and/or lower substituent group may optionally be different. In embodiments, when R^{1C} is substituted, it is substituted with at least one substituent group. In embodiments, when R^{1C} is substituted, it is substituted with at least one size-limited substituent group. In embodiments, when R^{1C} is substituted, it is substituted with at least one lower substituent group.

In embodiments, a substituted R^{1D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{1D} is substituted with a plurality of groups selected from substituent groups, size-limited substituent groups, and lower substituent groups; each substituent group, size-limited substituent group, and/or lower substituent group may optionally be different. In embodiments, when R^{1D} is substituted, it is substituted with at least one substituent group. In embodiments, when R^{1D} is substituted, it is substituted with at least one size-limited substituent group. In embodiments, when R^{1D} is substituted, it is substituted with at least one lower substituent group.

In embodiments, a substituted R² (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R² is substituted with a plurality of groups selected from substituent groups, size-limited substituent groups, and lower substituent groups; each substituent group, size-limited substituent group, and/or lower substituent group may optionally be different. In embodiments, when R² is substituted, it is substituted with at least one substituent group. In embodiments, when R² is substituted, it is substituted with at least one size-limited substituent group. In embodiments, when R² is substituted, it is substituted with at least one lower substituent group.

In embodiments, a substituted R^{2A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2A} is substituted with a plurality of groups selected from substituent groups, size-limited substituent groups, and lower substituent groups; each substituent group, size-limited substituent group, and/or lower substituent group may optionally be different. In embodiments, when R^{2A} is substituted, it is substituted with at least one substituent group. In embodiments, when R^{2A} is substituted, it is substituted with at least one size-limited substituent group. In embodiments, when R^{2A} is substituted, it is substituted with at least one lower substituent group.

In embodiments, a substituted R^{2B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2B} is substituted with a plurality of groups selected from substituent groups, size-limited substituent groups, and lower substituent groups; each substituent group, size-limited substituent group, and/or lower substituent group may optionally be different. In embodiments, when R^{2B} is substituted, it is substituted with at least one substituent group. In embodiments, when R^{2B} is substituted, it is substituted with at least one size-limited substituent group. In embodiments, when R^{2B} is substituted, it is substituted with at least one lower substituent group.

In embodiments, a substituted ring formed when R^{2A} and R^{2B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{2A} and R^{2B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{2A} and R^{2B} substituents bonded to the same nitrogen atom are joined is substituted, it is substituted with at least one substituent group. In embodiments, when the substituted ring formed when R^{2A} and R2^{B} substituents bonded to the same nitrogen atom are joined is substituted, it is substituted with at least one size-limited substituent group. In embodiments, when the substituted ring formed when R^{2A} and R^{2B} substituents bonded to the same nitrogen atom are joined is substituted, it is substituted with at least one lower substituent group.

In embodiments, a substituted R^{2C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2C} is substituted with a plurality of groups selected from substituent groups, size-limited substituent groups, and lower substituent groups; each substituent group, size-limited substituent group, and/or lower substituent group may optionally be different. In embodiments, when R^{2C} is substituted, it is substituted with at least one substituent group. In embodiments, when R^{2C} is substituted, it is substituted with at least one size-limited substituent group. In embodiments, when R^{2C} is substituted, it is substituted with at least one lower substituent group.

In embodiments, a substituted R^{2D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{2D} is substituted with a plurality of groups selected from substituent groups, size-limited substituent groups, and lower substituent groups; each substituent group, size-limited substituent group, and/or lower substituent group may optionally be different. In embodiments, when R^{2D} is substituted, it is substituted with at least one substituent group. In embodiments, when R^{2D} is substituted, it is substituted with at least one size-limited substituent group. In embodiments, when R^{2D} is substituted, it is substituted with at least one lower substituent group.

In embodiments, a substituted R³ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R³ is substituted with a plurality of groups selected from substituent groups, size-limited substituent groups, and lower substituent groups; each substituent group, size-limited substituent group, and/or lower substituent group may optionally be different. In embodiments, when R³ is substituted, it is substituted with at least one substituent group. In embodiments, when R³ is substituted, it is substituted with at least one size-limited substituent group. In embodiments, when R³ is substituted, it is substituted with at least one lower substituent group.

In embodiments, a substituted ring formed when two R3 substituents bonded to adjacent atoms are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed two R³ substituents bonded to adjacent atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed two R³ substituents bonded to adjacent atom are joined is substituted, it is substituted with at least one substituent group. In embodiments, when the substituted ring formed when two R³ substituents bonded to adjacent atom are joined is substituted, it is substituted with at least one size-limited substituent group. In embodiments, when the substituted ring formed when two R³ substituents bonded to adjacent atom are joined is substituted, it is substituted with at least one lower substituent group.

In embodiments, a substituted R^{3A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3A} is substituted with a plurality of groups selected from substituent groups, size-limited substituent groups, and lower substituent groups; each substituent group, size-limited substituent group, and/or lower substituent group may optionally be different. In embodiments, when R^{3A} is substituted, it is substituted with at least one substituent group. In embodiments, when R^{3A} is substituted, it is substituted with at least one size-limited substituent group. In embodiments, when R^{3A} is substituted, it is substituted with at least one lower substituent group.

In embodiments, a substituted R^{3B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3B} is substituted with a plurality of groups selected from substituent groups, size-limited substituent groups, and lower substituent groups; each substituent group, size-limited substituent group, and/or lower substituent group may optionally be different. In embodiments, when R^{3B} is substituted, it is substituted with at least one substituent group. In embodiments, when R^{3B} is substituted, it is substituted with at least one size-limited substituent group. In embodiments, when R^{3B} is substituted, it is substituted with at least one lower substituent group.

In embodiments, a substituted ring formed when R^{3A} and R^{3B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{3A} and R^{3B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{3A} and R^{3B} substituents bonded to the same nitrogen atom are joined is substituted, it is substituted with at least one substituent group. In embodiments, when the substituted ring formed when R^{3A} and R^{3B} substituents bonded to the same nitrogen atom are joined is substituted, it is substituted with at least one size-limited substituent group. In embodiments, when the substituted ring formed when R^{3A} and R^{3B} substituents bonded to the same nitrogen atom are joined is substituted, it is substituted with at least one lower substituent group.

In embodiments, a substituted R^{3C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3C} is substituted with a plurality of groups selected from substituent groups, size-limited substituent groups, and lower substituent groups; each substituent group, size-limited substituent group, and/or lower substituent group may optionally be different. In embodiments, when R^{3C} is substituted, it is substituted with at least one substituent group. In embodiments, when R^{3C} is substituted, it is substituted with at least one size-limited substituent group. In embodiments, when R^{3C} is substituted, it is substituted with at least one lower substituent group.

In embodiments, a substituted R^{3D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{3D} is substituted with a plurality of groups selected from substituent groups, size-limited substituent groups, and lower substituent groups; each substituent group, size-limited substituent group, and/or lower substituent group may optionally be different. In embodiments, when R^{3D} is substituted, it is substituted with at least one substituent group. In embodiments, when R^{3D} is substituted, it is substituted with at least one size-limited substituent group. In embodiments, when R^{3D} is substituted, it is substituted with at least one lower substituent group.

In embodiments, a substituted R⁴ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁴ is substituted with a plurality of groups selected from substituent groups, size-limited substituent groups, and lower substituent groups; each substituent group, size-limited substituent group, and/or lower substituent group may optionally be different. In embodiments, when R⁴ is substituted, it is substituted with at least one substituent group. In embodiments, when R⁴ is substituted, it is substituted with at least one size-limited substituent group. In embodiments, when R⁴ is substituted, it is substituted with at least one lower substituent group.

In embodiments, a substituted ring formed when two R⁴ substituents bonded to adjacent atoms are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed two R⁴ substituents bonded to adjacent atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed two R⁴ substituents bonded to adjacent atom are joined is substituted, it is substituted with at least one substituent group. In embodiments, when the substituted ring formed when two R⁴ substituents bonded to adjacent atom are joined is substituted, it is substituted with at least one size-limited substituent group. In embodiments, when the substituted ring formed when two R⁴ substituents bonded to adjacent atom are joined is substituted, it is substituted with at least one lower substituent group.

In embodiments, a substituted R^{4A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4A} is substituted with a plurality of groups selected from substituent groups, size-limited substituent groups, and lower substituent groups; each substituent group, size-limited substituent group, and/or lower substituent group may optionally be different. In embodiments, when R^{4A} is substituted, it is substituted with at least one substituent group. In embodiments, when R^{4A} is substituted, it is substituted with at least one size-limited substituent group. In embodiments, when R^{4A} is substituted, it is substituted with at least one lower substituent group.

In embodiments, a substituted R^{4B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4B} is substituted with a plurality of groups selected from substituent groups, size-limited substituent groups, and lower substituent groups; each substituent group, size-limited substituent group, and/or lower substituent group may optionally be different. In embodiments, when R^{4B} is substituted, it is substituted with at least one substituent group. In embodiments, when R^{4B} is substituted, it is substituted with at least one size-limited substituent group. In embodiments, when R^{4B} is substituted, it is substituted with at least one lower substituent group.

In embodiments, a substituted ring formed when R^{4A} and R^{4B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{4A} and R^{4B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{4A} and R^{4B} substituents bonded to the same nitrogen atom are joined is substituted, it is substituted with at least one substituent group. In embodiments, when the substituted ring formed when R^{4A} and R^{4B} substituents bonded to the same nitrogen atom are joined is substituted, it is substituted with at least one size-limited substituent group. In embodiments, when the substituted ring formed when R^{4A} and R^{4B} substituents bonded to the same nitrogen atom are joined is substituted, it is substituted with at least one lower substituent group.

In embodiments, a substituted R^{4C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4C} is substituted with a plurality of groups selected from substituent groups, size-limited substituent groups, and lower substituent groups; each substituent group, size-limited substituent group, and/or lower substituent group may optionally be different. In embodiments, when R^{4C} is substituted, it is substituted with at least one substituent group. In embodiments, when R^{4C} is substituted, it is substituted with at least one size-limited substituent group. In embodiments, when R^{4C} is substituted, it is substituted with at least one lower substituent group.

In embodiments, a substituted R^{4D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{4D} is substituted with a plurality of groups selected from substituent groups, size-limited substituent groups, and lower substituent groups; each substituent group, size-limited substituent group, and/or lower substituent group may optionally be different. In embodiments, when R^{4D} is substituted, it is substituted with at least one substituent group. In embodiments, when R^{4D} is substituted, it is substituted with at least one size-limited substituent group. In embodiments, when R^{4D} is substituted, it is substituted with at least one lower substituent group.

In embodiments, a substituted R¹⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R¹⁰ is substituted with a plurality of groups selected from substituent groups, size-limited substituent groups, and lower substituent groups; each substituent group, size-limited substituent group, and/or lower substituent group may optionally be different. In embodiments, when R¹⁰ is substituted, it is substituted with at least one substituent group. In embodiments, when R¹⁰ is substituted, it is substituted with at least one size-limited substituent group. In embodiments, when R¹⁰ is substituted, it is substituted with at least one lower substituent group.

In embodiments, a substituted ring formed when two R¹⁰ substituents bonded to adjacent atoms are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed two R¹⁰ substituents bonded to adjacent atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed two R¹⁰ substituents bonded to adjacent atom are joined is substituted, it is substituted with at least one substituent group. In embodiments, when the substituted ring formed when two R¹⁰ substituents bonded to adjacent atom are joined is substituted, it is substituted with at least one size-limited substituent group. In embodiments, when the substituted ring formed when two R¹⁰ substituents bonded to adjacent atom are joined is substituted, it is substituted with at least one lower substituent group.

In embodiments, a substituted R^{10A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10A} is substituted with a plurality of groups selected from substituent groups, size-limited substituent groups, and lower substituent groups; each substituent group, size-limited substituent group, and/or lower substituent group may optionally be different. In embodiments, when R^{10A} is substituted, it is substituted with at least one substituent group. In embodiments, when R^{10A} is substituted, it is substituted with at least one size-limited substituent group. In embodiments, when R^{10A} is substituted, it is substituted with at least one lower substituent group.

In embodiments, a substituted R^{10B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10B} is substituted with a plurality of groups selected from substituent groups, size-limited substituent groups, and lower substituent groups; each substituent group, size-limited substituent group, and/or lower substituent group may optionally be different. In embodiments, when R^{10B} is substituted, it is substituted with at least one substituent group. In embodiments, when R^{10B} is substituted, it is substituted with at least one size-limited substituent group. In embodiments, when R^{10B} is substituted, it is substituted with at least one lower substituent group.

In embodiments, a substituted ring formed when R^{10A} and R^{10B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{10A} and R^{10B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{10A} and R^{10B} substituents bonded to the same nitrogen atom are joined is substituted, it is substituted with at least one substituent group. In embodiments, when the substituted ring formed when R^{10A} and R^{10B} substituents bonded to the same nitrogen atom are joined is substituted, it is substituted with at least one size-limited substituent group. In embodiments, when the substituted ring formed when R^{10A} and R^{10B} substituents bonded to the same nitrogen atom are joined is substituted, it is substituted with at least one lower substituent group.

In embodiments, a substituted R^{10C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10C} is substituted with a plurality of groups selected from substituent groups, size-limited substituent groups, and lower substituent groups; each substituent group, size-limited substituent group, and/or lower substituent group may optionally be different. In embodiments, when R^{10C} is substituted, it is substituted with at least one substituent group. In embodiments, when R^{10C} is substituted, it is substituted with at least one size-limited substituent group. In embodiments, when R^{10C} is substituted, it is substituted with at least one lower substituent group.

In embodiments, a substituted R^{10D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10D} is substituted with a plurality of groups selected from substituent groups, size-limited substituent groups, and lower substituent groups; each substituent group, size-limited substituent group, and/or lower substituent group may optionally be different. In embodiments, when R^{10D} is substituted, it is substituted with at least one substituent group. In embodiments, when R^{10D} is substituted, it is substituted with at least one size-limited substituent group. In embodiments, when R^{10D} is substituted, it is substituted with at least one lower substituent group.

In embodiments, a substituted R^{10.A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10.A} is substituted with a plurality of groups selected from substituent groups, size-limited substituent groups, and lower substituent groups; each substituent group, size-limited substituent group, and/or lower substituent group may optionally be different. In embodiments, when R¹⁰^{.}^{A} is substituted, it is substituted with at least one substituent group. In embodiments, when R^{10.A} is substituted, it is substituted with at least one size-limited substituent group. In embodiments, when R^{10.A} is substituted, it is substituted with at least one lower substituent group.

In embodiments, a substituted R^{10.B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10.B} is substituted with a plurality of groups selected from substituent groups, size-limited substituent groups, and lower substituent groups; each substituent group, size-limited substituent group, and/or lower substituent group may optionally be different. In embodiments, when R¹⁰^{.}^{B} is substituted, it is substituted with at least one substituent group. In embodiments, when R^{10.B} is substituted, it is substituted with at least one size-limited substituent group. In embodiments, when R^{10.B} is substituted, it is substituted with at least one lower substituent group.

In embodiments, a substituted R^{10.C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10.C} is substituted with a plurality of groups selected from substituent groups, size-limited substituent groups, and lower substituent groups; each substituent group, size-limited substituent group, and/or lower substituent group may optionally be different. In embodiments, when R^{10.C} is substituted, it is substituted with at least one substituent group. In embodiments, when R^{10.C} is substituted, it is substituted with at least one size-limited substituent group. In embodiments, when R^{10.C} is substituted, it is substituted with at least one lower substituent group.

In embodiments, a substituted R^{10.D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10.D} is substituted with a plurality of groups selected from substituent groups, size-limited substituent groups, and lower substituent groups; each substituent group, size-limited substituent group, and/or lower substituent group may optionally be different. In embodiments, when R¹⁰^{.}^{D} is substituted, it is substituted with at least one substituent group. In embodiments, when R^{10.D} is substituted, it is substituted with at least one size-limited substituent group. In embodiments, when R^{10.D} is substituted, it is substituted with at least one lower substituent group.

In embodiments, a substituted R^{10.E} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{10.E} is substituted with a plurality of groups selected from substituent groups, size-limited substituent groups, and lower substituent groups; each substituent group, size-limited substituent group, and/or lower substituent group may optionally be different. In embodiments, when R¹⁰^{.}^{E} is substituted, it is substituted with at least one substituent group. In embodiments, when R^{10.E} is substituted, it is substituted with at least one size-limited substituent group. In embodiments, when R^{10.E} is substituted, it is substituted with at least one lower substituent group.

In embodiments, a substituted R²⁰ (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R²⁰ is substituted with a plurality of groups selected from substituent groups, size-limited substituent groups, and lower substituent groups; each substituent group, size-limited substituent group, and/or lower substituent group may optionally be different. In embodiments, when R²⁰ is substituted, it is substituted with at least one substituent group. In embodiments, when R²⁰ is substituted, it is substituted with at least one size-limited substituent group. In embodiments, when R²⁰ is substituted, it is substituted with at least one lower substituent group.

In embodiments, a substituted ring formed when two R²⁰ substituents bonded to adjacent atoms are joined (e.g., substituted cycloalkyl, substituted heterocycloalkyl, substituted aryl, and/or substituted heteroaryl) is substituted with at least one substituent group, size-limited substituent group, or lower substituent group; wherein if the substituted ring formed two R²⁰ substituents bonded to adjacent atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed two R²⁰ substituents bonded to adjacent atom are joined is substituted, it is substituted with at least one substituent group. In embodiments, when the substituted ring formed when two R²⁰ substituents bonded to adjacent atom are joined is substituted, it is substituted with at least one size-limited substituent group. In embodiments, when the substituted ring formed when two R²⁰ substituents bonded to adjacent atom are joined is substituted, it is substituted with at least one lower substituent group.

In embodiments, a substituted R^{20A} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20A} is substituted with a plurality of groups selected from substituent groups, size-limited substituent groups, and lower substituent groups; each substituent group, size-limited substituent group, and/or lower substituent group may optionally be different. In embodiments, when R^{20A} is substituted, it is substituted with at least one substituent group. In embodiments, when R^{20A} is substituted, it is substituted with at least one size-limited substituent group. In embodiments, when R^{20A} is substituted, it is substituted with at least one lower substituent group.

In embodiments, a substituted R^{20B} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20B} is substituted with a plurality of groups selected from substituent groups, size-limited substituent groups, and lower substituent groups; each substituent group, size-limited substituent group, and/or lower substituent group may optionally be different. In embodiments, when R^{20B} is substituted, it is substituted with at least one substituent group. In embodiments, when R^{20B} is substituted, it is substituted with at least one size-limited substituent group. In embodiments, when R^{20B} is substituted, it is substituted with at least one lower substituent group.

In embodiments, a substituted ring formed when R^{20A} and R^{20B} substituents bonded to the same nitrogen atom are joined (e.g., substituted heterocycloalkyl and/or substituted heteroaryl) is substituted with at least one substituent group, size-limited substituent group, or lower substituent group; wherein if the substituted ring formed when R^{20A} and R^{20B} substituents bonded to the same nitrogen atom are joined is substituted with a plurality of groups selected from substituent groups, size-limited substituent groups, and lower substituent groups; each substituent group, size-limited substituent group, and/or lower substituent group may optionally be different. In embodiments, when the substituted ring formed when R^{20A} and R^{20B} substituents bonded to the same nitrogen atom are joined is substituted, it is substituted with at least one substituent group. In embodiments, when the substituted ring formed when R^{20A} and R^{20B} substituents bonded to the same nitrogen atom are joined is substituted, it is substituted with at least one size-limited substituent group. In embodiments, when the substituted ring formed when R^{20A} and R^{20B} substituents bonded to the same nitrogen atom are joined is substituted, it is substituted with at least one lower substituent group.

In embodiments, a substituted R^{20C} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20C} is substituted with a plurality of groups selected from substituent groups, size-limited substituent groups, and lower substituent groups; each substituent group, size-limited substituent group, and/or lower substituent group may optionally be different. In embodiments, when R^{20C} is substituted, it is substituted with at least one substituent group. In embodiments, when R^{20C} is substituted, it is substituted with at least one size-limited substituent group. In embodiments, when R^{20C} is substituted, it is substituted with at least one lower substituent group.

In embodiments, a substituted R^{20D} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{20D} is substituted with a plurality of groups selected from substituent groups, size-limited substituent groups, and lower substituent groups; each substituent group, size-limited substituent group, and/or lower substituent group may optionally be different. In embodiments, when R^{20D} is substituted, it is substituted with at least one substituent group. In embodiments, when R^{20D} is substituted, it is substituted with at least one size-limited substituent group. In embodiments, when R^{20D} is substituted, it is substituted with at least one lower substituent group.

In embodiments, a substituted L¹ (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L¹ is substituted with a plurality of groups selected from substituent groups, size-limited substituent groups, and lower substituent groups; each substituent group, size-limited substituent group, and/or lower substituent group may optionally be different. In embodiments, when L¹ is substituted, it is substituted with at least one substituent group. In embodiments, when L¹ is substituted, it is substituted with at least one size-limited substituent group. In embodiments, when L¹ is substituted, it is substituted with at least one lower substituent group.

In embodiments, a substituted R^{L1} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{L1} is substituted with a plurality of groups selected from substituent groups, size-limited substituent groups, and lower substituent groups; each substituent group, size-limited substituent group, and/or lower substituent group may optionally be different. In embodiments, when R^{L1} is substituted, it is substituted with at least one substituent group. In embodiments, when R^{L1} is substituted, it is substituted with at least one size-limited substituent group. In embodiments, when R^{L1} is substituted, it is substituted with at least one lower substituent group.

In embodiments, a substituted L2 (e.g., substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted L2 is substituted with a plurality of groups selected from substituent groups, size-limited substituent groups, and lower substituent groups; each substituent group, size-limited substituent group, and/or lower substituent group may optionally be different. In embodiments, when L² is substituted, it is substituted with at least one substituent group. In embodiments, when L² is substituted, it is substituted with at least one size-limited substituent group. In embodiments, when L² is substituted, it is substituted with at least one lower substituent group.

In embodiments, a substituted R^{L2} (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R^{L2} is substituted with a plurality of groups selected from substituent groups, size-limited substituent groups, and lower substituent groups; each substituent group, size-limited substituent group, and/or lower substituent group may optionally be different. In embodiments, when R^{L2} is substituted, it is substituted with at least one substituent group. In embodiments, when R^{L2} is substituted, it is substituted with at least one size-limited substituent group. In embodiments, when R^{L2} is substituted, it is substituted with at least one lower substituent group.

In embodiments, the compound is a compound described herein. In embodiments, the compound, or salt (e.g., pharmaceutically acceptable salt) thereof, is the compound. In embodiments, the compound, or a salt (e.g., pharmaceutically acceptable salt) thereof, is the salt (e.g., pharmaceutically acceptable salt) of the compound. In embodiments, the compound, or a salt (e.g., pharmaceutically acceptable salt) thereof, is the pharmaceutically acceptable salt of the compound.

In embodiments, R¹, R², L², R³, R⁴, Ring A, and z3 are as described herein, including in embodiments; and L¹ is a bond, —N(R^{L1})—, —O—, —S—, —SO₂—, —C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene (e.g., C₁—C₈, C₁—C₆, or C₁—C₄) or substituted or unsubstituted heteroalkylene (e.g., 2 to 8 membered, 2 to 6 membered, or 2 to 4 membered). In embodiments, L¹ is a bond, —N(R^{L1})—, —O—, —S—, —SO₂—, —C(O)—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene (e.g., C₁—C₈, C₁—C₆, or C₁—C₄) or substituted or unsubstituted heteroalkylene (e.g., 2 to 8 membered, 2 to 6 membered, or 2 to 4 membered). In embodiments, L¹ is not —C(O)N(R^{L1})—. In embodiments, L¹ is not —C(O)NH—. In embodiments, L¹ is not —C(O)N(R^{L1})—, wherein N is bonded directly to R¹. In embodiments, L¹ is not —C(O)NH—, wherein N is bonded directly to R¹.

In embodiments, L¹, R², L², R³, R⁴, Ring A, and z3 are as described herein, including in embodiments; and R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or unsubstituted heteroaryl (e.g., 5 to 10 membered, 5 to 9 membered, or 5 to 6 membered). In embodiments, R¹ is independently hydrogen, halogen, —CX¹₃, -CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), or substituted or unsubstituted aryl (e.g., C₆—C₁₀, C₁₀, or phenyl). In embodiments, R¹ is not substituted heteroaryl. In embodiments, R¹ is not substituted 6 membered heteroaryl. In embodiments, R¹ is not substituted pyridyl. In embodiments, R¹ is not substituted 2-pyridyl. In embodiments, R¹ is not OH-substituted 2-pyridyl. In embodiments, R¹ is not

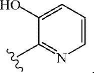

In embodiments, R¹, L¹, R², R³, R⁴, Ring A, and z3 are as described herein, including in embodiments; and L2 is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted alkylene (e.g., C₁—C₈, C₁—C₆, or C₁—C₄) or substituted or unsubstituted heteroalkylene (e.g., 2 to 8 membered, 2 to 6 membered, or 2 to 4 membered). In embodiments, L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2}) SO₂—, or substituted or unsubstituted heteroalkylene (e.g., 2 to 8 membered, 2 to 6 membered, or 2 to 4 membered). In embodiments, L² is not unsubstituted alkylene (e.g., C₁—C₈, C₁—C₆, or C₁—C₄). In embodiments, L² is not unsubstituted C₁—C₄ alkylene. In embodiments, L² is not unsubstituted methylene.

In embodiments, R¹, L¹, L², R³, R⁴, Ring A, and z3 are as described herein, including in embodiments; and R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted heteroaryl (e.g., 5 to 10 membered, 5 to 9 membered, or 5 to 6 membered). In embodiments, R2 is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), or substituted or unsubstituted aryl (e.g., C₆—C₁₀, C₁₀, or phenyl). In embodiments, R² is not unsubstituted heteroaryl (e.g., 5 to 10 membered, 5 to 9 membered, or 5 to 6 membered). In embodiments, R² is not unsubstituted 5 membered heteroaryl. In embodiments, R² is not unsubstituted furanyl. In embodiments, R² is not unsubstituted 2-furanyl.

In embodiments, R¹, L¹, R², L², R³, R⁴, and z3 are as described herein, including in embodiments; and Ring A is C₅ cycloalkyl, 5 to 6 membered heterocycloalkyl, phenyl, or 5 to 6 membered heteroaryl. In embodiments, Ring A is 5 to 6 membered heterocycloalkyl, phenyl, or 5 to 6 membered heteroaryl. In embodiments, Ring A is not C₆ cycloalkyl. In embodiments, Ring A is not C₅—C₆ cycloalkyl.

In embodiments, R¹, L¹, R², L², R³, R⁴, and Ring A are as described herein, including in embodiments; and z3 is independently an integer from 1 to 8. In embodiments, z3 is not 0.

In embodiments, R¹, L¹, R², L², R³, Ring A, and z3 are as described herein, including in embodiments; and R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, or —NR^{4A}R^{4B}. In embodiments, R⁴ is not -OR^{4D}. In embodiments, R⁴ is not —OH.

In embodiments, R¹, L¹, R², L², R³, R⁴, Ring A, and z3 are as described herein, including in embodiments; and R^{4D} is independently —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered). In embodiments, R^{4D} is not hydrogen.

In embodiments, R¹, L¹, R², L², R³, R⁴, Ring A, and z3 are as described herein, including in embodiments; and R^{L1} is independently —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered). In embodiments, R^{L1} is not hydrogen.

In embodiments, R¹ is not

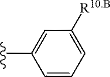

and R^{10.C} is as described herein, including in embodiments. In embodiments, R¹ is not

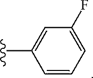

In embodiments, R¹ is not

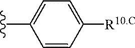

and R^{10.C} is as described herein, including in embodiments. In embodiments, R¹ is not

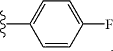

In embodiments, —L²—R² is not hydrogen, —CH₃, —CH₂—(unsubstituted phenyl), or unsubstituted phenyl. In embodiments, —L²—R² is not hydrogen. In embodiments, —L²—R² is not —CH₃. In embodiments, —L²—R² is not —CH₂—(unsubstituted phenyl). In embodiments, —L²—R² is not unsubstituted phenyl.

In embodiments, R³ is not halogen or substituted or unsubstituted 3 to 6 membered heterocycloalkyl. In embodiments, R³ is not —Br or substituted or unsubstituted piperazinyl.

In embodiments, the compound is not

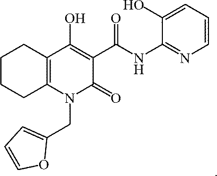

In embodiments, the compound is not:

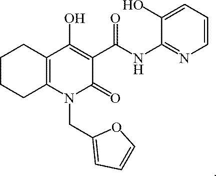

In embodiments, the compound is not:

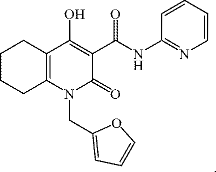

In embodiments, the compound is not:

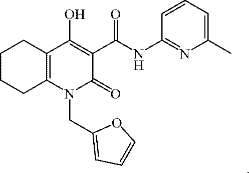

In embodiments, the compound is not:

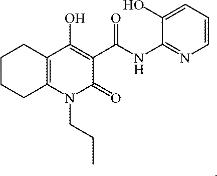

In embodiments, the compound is not:

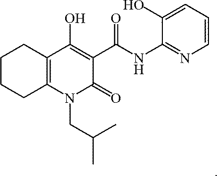

In embodiments, the compound is not:

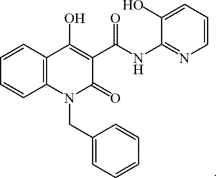

In embodiments, the compound is not:

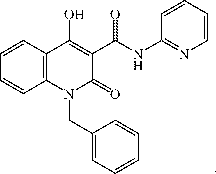 [SMILES: Cc1cccnc1NC(=O)c1c(O)c2ccccc2n(Cc2ccccc2)c1=O ; ". In"]

In embodiments, the compound is not:

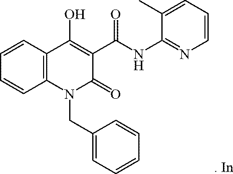

embodiments, the compound is not:

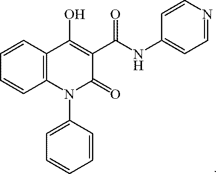

In embodiments, the compound is not:

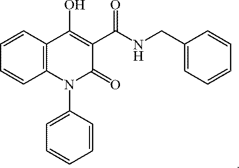

In embodiments, the compound is not:

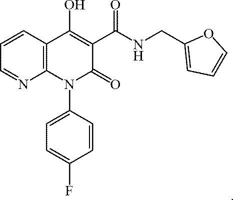

In embodiments, the compound is not:

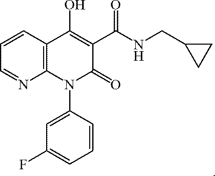

In embodiments, the compound is not:

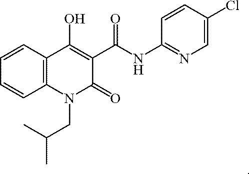

In embodiments, the compound is not:

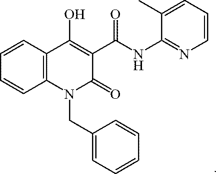

In embodiments, the compound is not:

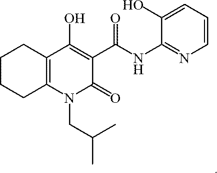 [SMILES: Cc1cccnc1NC(=O)c1c(O)c2ccc(Br)cc2n(CC(C)C)c1=O ; ". In"]

In embodiments, the compound is not:

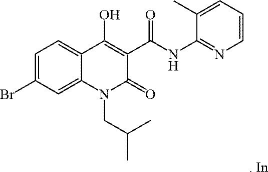 [SMILES: CC(C)Cn1c(=O)c(C(=O)Nc2cccc(F)c2)c(O)c2c(Br)cccc21 ; "."]

embodiments, the compound is not:

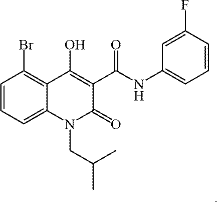 [SMILES: CC(C)Cn1c(=O)c(C(=O)Nc2cccc(F)c2)c(O)c2cc(Br)ccc21 ; "."]

In embodiments, the compound is not:

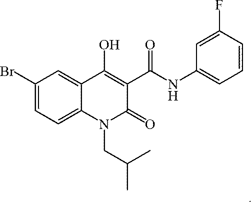

In embodiments, the compound is not:

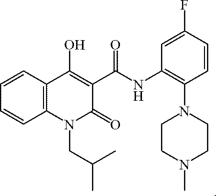 [SMILES: Cc1cccnc1NC(=O)c1c(O)c2cc(Br)ccc2n(CC(C)C)c1=O]

In embodiments, the compound is not:

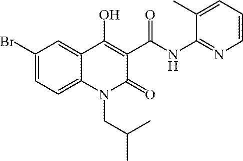 [SMILES: CC(C)Cn1c(=O)c(C(=O)Nc2cccc(F)c2)c(O)c2ccc(N3CCN(C)CC3)cc21 ; ". In"]

. In embodiments, the compound is not:

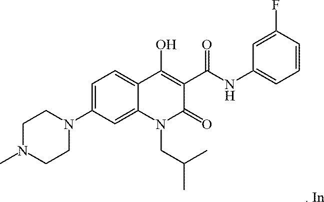 [SMILES: Cc1cccnc1NC(=O)c1c(O)c2cc(N3CCN(C)CC3)ccc2n(CC(C)C)c1=O ; "."]

embodiments, the compound is not:

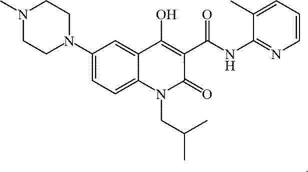

In embodiments, the compound is not:

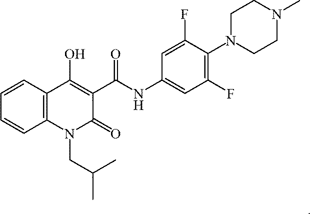

In embodiments, the compound is not:

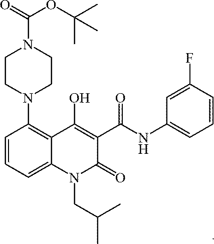

In embodiments, the compound is not:

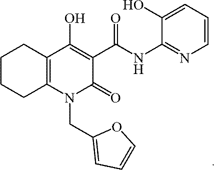

In embodiments, the compound is not:

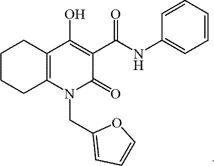

In embodiments, the compound is not:

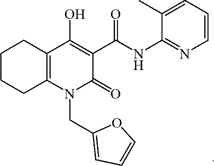

In embodiments, the compound is not:

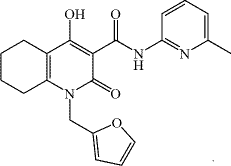

In embodiments, the compound is not:

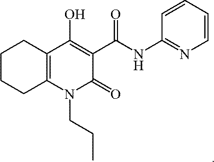

In embodiments, the compound is not:

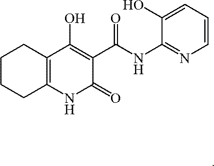

In embodiments, the compound is not:

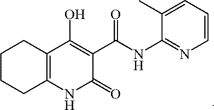

In embodiments, the compound is not:

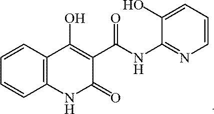 [SMILES: Cn1c(=O)c(C(=O)Nc2ncccc2O)c(O)c2ccccc21 ; ". In"]

In embodiments, the compound is not:

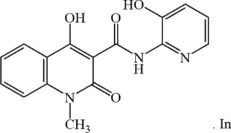 [SMILES: Cc1ccnc(NC(=O)c2c(O)c3ccccc3n(Cc3ccccc3)c2=O)c1 ; "."]

embodiments, the compound is not:

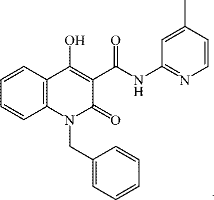 [SMILES: Cc1ccc(NC(=O)c2c(O)c3ccccc3n(Cc3ccccc3)c2=O)nc1 ; "."]

In embodiments, the compound is not:

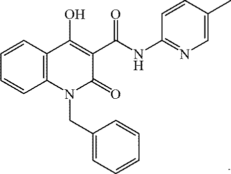 [SMILES: O=C(Nc1cccnc1)c1c(O)c2ccccc2n(-c2ccccc2)c1=O ; "."]

In embodiments, the compound is not:

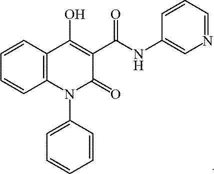 [SMILES: O=C(Nc1ccc(F)cc1)c1c(O)c2cccnc2n(-c2ccccc2)c1=O ; "."]

In embodiments, the compound is not:

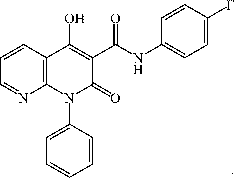 [SMILES: CCn1c(=O)c(C(=O)Nc2ccccn2)c(O)c2ccccc21 ; "."]

In embodiments, the compound is not:

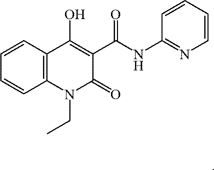 [SMILES: CCn1c(=O)c(C(=O)Nc2ccc(Cl)cn2)c(O)c2ccccc21 ; "."]

In embodiments, the compound is not:

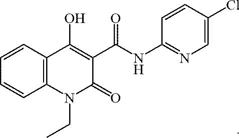 [SMILES: Cc1ccnc(NC(=O)c2c(O)c3ccccc3n(Cc3ccccc3)c2=O)c1 ; "."]

In embodiments, the compound is not:

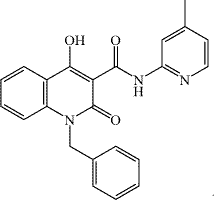 [SMILES: Cc1ccc(NC(=O)c2c(O)c3ccccc3n(Cc3ccccc3)c2=O)nc1 ; "."]

In embodiments, the compound is not:

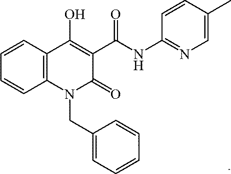 [SMILES: Cc1cccc(NC(=O)c2c(O)c3ccccc3n(Cc3ccccc3)c2=O)n1 ; "."]

In embodiments, the compound is not:

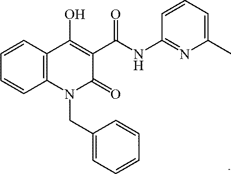 [SMILES: CCCn1c2c(c(O)c(C(=O)Nc3ccccn3)c1=O)CCCC2 ; "."]

In embodiments, the compound is not:

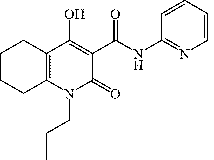 [SMILES: Cc1cccnc1NC(=O)c1c(O)c2cc(Cl)ccc2n(CC(C)C)c1=O ; "."]

In embodiments, the compound is not:

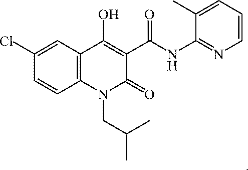 [SMILES: Cc1cccnc1NC(=O)c1c(O)c2cc(C(F)(F)F)ccc2n(CC(C)C)c1=O ; "."]

In embodiments, the compound is not:

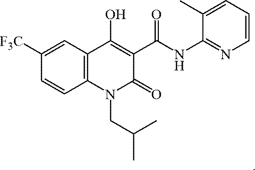 [SMILES: CC(C)Cn1c(=O)c(C(=O)Nc2ncccc2O)c(O)c2ccccc21 ; "."]

In embodiments, the compound is not:

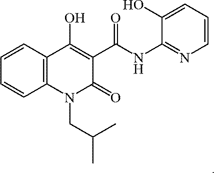 [SMILES: CC(=O)Nc1ccccc1NC(=O)c1c(O)c2ccccc2n(CC(C)C)c1=O ; "."]

In embodiments, the compound is not:

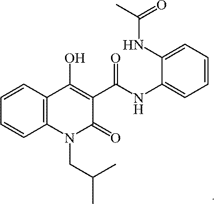 [SMILES: CC(C)Cn1c(=O)c(C(=O)Nc2ccncc2O)c(O)c2ccccc21 ; "."]

In embodiments, the compound is not:

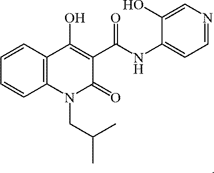 [SMILES: CC(C)Cn1c(=O)c(C(=O)Nc2ncc(Cl)cc2O)c(O)c2ccccc21 ; "."]

In embodiments, the compound is not:

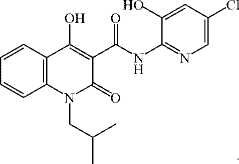 [SMILES: CC(C)Cn1c(=O)c(C(=O)Nc2ncccc2OCc2ccccc2)c(O)c2ccccc21 ; "."]

In embodiments, the compound is not:

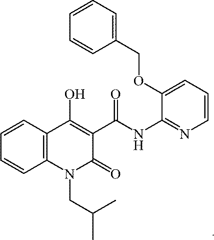 [SMILES: COc1cccnc1NC(=O)c1c(O)c2ccccc2n(CC(C)C)c1=O ; ". In"]

In embodiments, the compound is not:

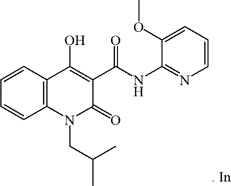 [SMILES: COc1ccccc1NC(=O)c1c(O)c2ccccc2n(CC(C)C)c1=O ; "."]

embodiments, the compound is not:

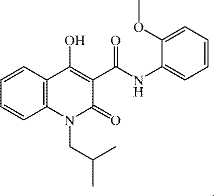 [SMILES: CC(C)Cn1c(=O)c(C(=O)Nc2cccc(F)c2)c(O)c2ccc(N3CCN(C)CC3)cc21 ; "."]

In embodiments, the compound is not:

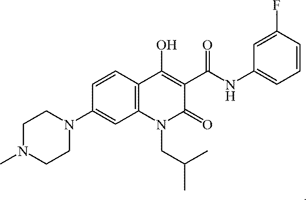

In embodiments, the compound has the formula:

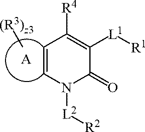

or a salt (e.g., pharmaceutically acceptable salt) thereof, wherein; L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene (e.g., C₁—C₈, C₁—C₆, or C₁—C₄) or substituted or unsubstituted heteroalkylene (e.g., 2 to 8 membered, 2 to 6 membered, or 2 to 4 membered); R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene (e.g., C₁—C₈, C₁—C₆, or C₁—C₄) or substituted or unsubstituted heteroalkylene (e.g., 2 to 8 membered, 2 to 6 membered, or 2 to 4 membered); R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); Ring A is 5 to 6 membered heterocycloalkyl, phenyl, or 5 to 6 membered heteroaryl; R³ is independently halogen, oxo, —CX³₃, —CHX³₂, —CH₂X³, —OCX³₃, —OCH₂X³, —OCHX³₂, —CN, —SOₙ₃R^{3D}, -SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); two adjacent R³ substituents may optionally be joined to form a substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); z3 is independently an integer from 0 to 8; R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D}; R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl (e.g., C₁—C₈, C₁—C₆, or C₁—C₄), substituted or unsubstituted heteroalkyl (e.g., 2 to 8 membered, 2 to 6 membered, or 2 to 4 membered), substituted or unsubstituted cycloalkyl (e.g., C₃—C₈, C₃—C₆, or C₅—C₆), substituted or unsubstituted heterocycloalkyl (e.g., 3 to 8 membered, 3 to 6 membered, or 5 to 6 membered), substituted or unsubstituted aryl (e.g., C₆—C₁₀, C₁₀, or phenyl), or substituted or unsubstituted heteroaryl (e.g., 5 to 10 membered, 5 to 9 membered, or 5 to 6 membered); R^{1A} and R^{1B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered) ; R^{2A} and R^{2B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered); R^{3A} and R^{3B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered) ; R^{4A} and R^{4B} substituents bonded to the same nitrogen atom may optionally be joined to form a substituted or unsubstituted heterocycloalkyl (e.g., 3 to 8 membered, 3 to 6 membered, or 5 to 6 membered) or substituted or unsubstituted heteroaryl (e.g., 5 to 10 membered, 5 to 9 membered, or 5 to 6 membered); X¹, X², X³, and X⁴, are independently —F, —Cl, —Br, or —I; n1, n2, and n3 are independently an integer from 0 to 4; m1, m2, m3, v1, v2, and v3 are independently 1 or 2.

In embodiments, the compound has the formula:

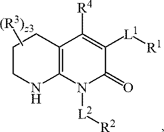

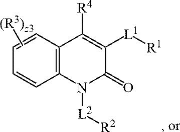

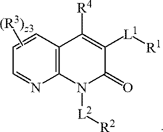

R¹, L¹, R², L², R³, R⁴, and z3 are as described herein, including in embodiments.

In embodiments, the compound has the formula:

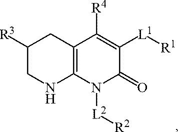

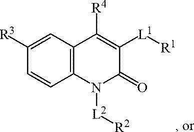

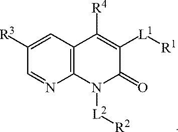

R¹, L¹, R², L², R³, and R⁴³ are as described herein, including in embodiments.

In embodiments, the compound has the formula:

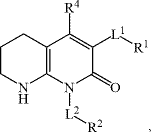

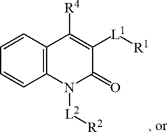

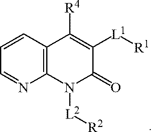

R¹, L¹, R², L², and R⁴ are as described herein, including in embodiments.

In embodiments, the compound has the formula:

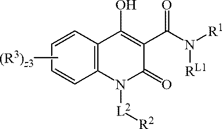

and R¹, R^{L1}, L2, R², R³, and z3 are as described herein, including in embodiments.

In embodiments, the compound has the formula:

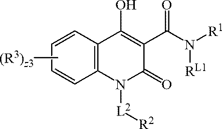

and R^{L1}, L2, R², R³, and z3 are as described herein, including in embodiments; R¹ is

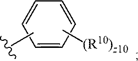

R¹⁰ is independently halogen, substituted or unsubstituted cycloalkyl, or substituted or unsubstituted heterocycloalkyl; z10 is an integer from 1 to 5; and wherein —L²—R² is not hydrogen. In embodiments, at least one R¹⁰ is independently a substituted or unsubstituted cycloalkyl or substituted or unsubstituted heterocycloalkyl. In embodiments, R¹⁰ is independently a substituted or unsubstituted C₆ cycloalkyl or substituted or unsubstituted 6 membered heterocycloalkyl. In embodiments, z3 is 0.

In embodiments, the compound has the formula:

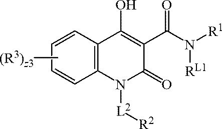

and R^{L1}, L2, R², R³, and z3 are as described herein, including in embodiments; R¹ is

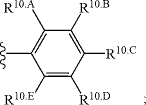

R^{10.A}, R^{10.B}, R^{10.C}, R^{10.D}, and R^{10.E} are as described herein, including in embodiments; wherein —L²—R² is not hydrogen; and wherein at least one of R^{10.A}, R^{10.B}, R^{10.C},R^{10.D}, or R¹⁰ is a substituted or unsubstituted cycloalkyl or substituted or unsubstituted heterocycloalkyl. In embodiments, at least one of R³, R^{10.A}, R¹⁰ R^{10.C}, R^{10.D}, or R^{10.E} is a substituted or unsubstituted cycloalkyl or substituted or unsubstituted heterocycloalkyl.

In embodiments, the compound has the formula:

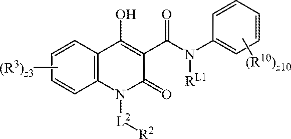

and L², R2, R³, z3, R^{L1}, R¹⁰, and z10 are as described herein, including in embodiments.

In embodiments, the compound has the formula:

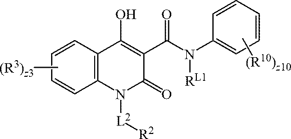

and L², R2, R³, z3, R^{L1}, R¹⁰, and z10 are as described herein, including in embodiments; R¹⁰ is independently halogen, substituted or unsubstituted cycloalkyl, or substituted or unsubstituted heterocycloalkyl; wherein at least one R¹⁰ is independently a substituted or unsubstituted cycloalkyl or substituted or unsubstituted heterocycloalkyl; and wherein —L²—R² is not hydrogen. In embodiments, R¹⁰ is independently a substituted or unsubstituted C₆ cycloalkyl or substituted or unsubstituted 6 membered heterocycloalkyl. In embodiments, z3 is 0.

In embodiments, the compound has the formula:

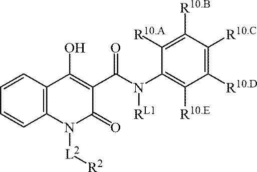

and L2, R², and R^{L1} are as described herein, including in embodiments. R^{10.A}, R^{10.B}, R^{10.C}, R^{10.D}, and R^{10.E} are independently hydrogen or any value of R¹⁰ described herein, including in embodiments. In embodiments, R^{10.C} is a substituted or unsubstituted cycloalkyl or substituted or unsubstituted heterocycloalkyl. In embodiments, R^{10.C} is a substituted or unsubstituted C₆ cycloalkyl or substituted or unsubstituted 6 membered heterocycloalkyl. In embodiments, R^{10.C} is a substituted or unsubstituted 6 membered heterocycloalkyl. In embodiments, R^{10.C} is a substituted or unsubstituted piperazinyl.

In embodiments, the compound has the formula:

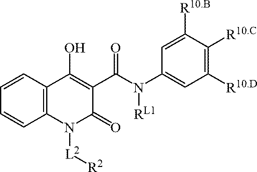

and L2, R², and R^{L1} are as described herein, including in embodiments. R^{10.B}, R^{10.C}, and R^{10.D} are independently hydrogen or any value of R¹⁰ described herein, including in embodiments. In embodiments, R^{10.C} is a substituted or unsubstituted cycloalkyl or substituted or unsubstituted heterocycloalkyl. In embodiments, R^{10.C} is a substituted or unsubstituted C₆ cycloalkyl or substituted or unsubstituted 6 membered heterocycloalkyl. In embodiments, R^{10.C} is a substituted or unsubstituted 6 membered heterocycloalkyl. In embodiments, R^{10.C} is a substituted or unsubstituted piperazinyl.

In embodiments, the compound is useful as a comparator compound. In embodiments, the comparator compound can be used to assess the activity of a test compound as set forth in an assay described herein (e.g., in the examples section, figures, or tables).

In embodiments, the compound is a compound described herein. In embodiments, the compound, or salt (e.g., pharmaceutically acceptable salt) thereof, is the compound. In embodiments, the compound, or a salt (e.g., pharmaceutically acceptable salt) thereof, is the salt (e.g., pharmaceutically acceptable salt) of the compound. In embodiments, the compound, or a salt (e.g., pharmaceutically acceptable salt) thereof, is the pharmaceutically acceptable salt of the compound.

### III. Pharmaceutical Compositions

In an aspect is provided a pharmaceutical composition including a compound described herein, or a salt (e.g., pharmaceutically acceptable salt) thereof, and a pharmaceutically acceptable excipient. In embodiments, the compound as described herein is included in a therapeutically effective amount. In embodiments, the compound as described herein is included in an effective amount.

In embodiments of the pharmaceutical compositions, the compound, or salt (e.g., pharmaceutically acceptable salt) thereof, is included in a therapeutically effective amount. In embodiments of the pharmaceutical compositions, the compound, or salt (e.g., pharmaceutically acceptable salt) thereof, is a compound. In embodiments of the pharmaceutical compositions, the compound, or salt (e.g., pharmaceutically acceptable salt) thereof, is a salt (e.g., pharmaceutically acceptable salt) of the compound. In embodiments of the pharmaceutical compositions, the compound, or salt (e.g., pharmaceutically acceptable salt) thereof, is a pharmaceutically acceptable salt of the compound.

In embodiments of the pharmaceutical compositions, the pharmaceutical composition includes a second agent (e.g., therapeutic agent). In embodiments of the pharmaceutical compositions, the pharmaceutical composition includes a second agent (e.g., therapeutic agent) in a therapeutically effective amount. In embodiments of the pharmaceutical compositions, the second agent is an agent for treating cancer. In embodiments of the pharmaceutical compositions, the second agent is an anti-cancer agent. In embodiments, the administering does not include administration of any active agent other than the recited active agent (e.g., a compound described herein). In embodiments, the second agent is included in an effective amount.

### IV. Methods of Use

In an aspect is provided a method of decreasing the level of Notch (e.g., one or more of Notch 1, Notch 2, Notch 3, and/or Notch 4) protein activity in a subject, the method including administering a compound described herein, or a salt (e.g., pharmaceutically acceptable salt) thereof, to the subject. In embodiments, the compound is administered in a therapeutically effective amount. In embodiments, the compound as described herein is included in an effective amount. In embodiments, the Notch is Notch 1. In embodiments, the Notch is Notch 2. In embodiments, the Notch is Notch 3. In embodiments, the Notch is Notch 4. In embodiments, the Notch is Notch 1 and Notch 2. In embodiments, the Notch is Notch 1 and Notch 3. In embodiments, the Notch is Notch 1 and Notch 4. In embodiments, the Notch is Notch 2 and Notch 3. In embodiments, the Notch is Notch 2 and Notch 4. In embodiments, the Notch is Notch 3 and Notch 4. In embodiments, the Notch is Notch 1, Notch 2, and Notch 3. In embodiments, the Notch is Notch 1, Notch 2, and Notch 4. In embodiments, the Notch is Notch 1, Notch 3, and Notch 4. In embodiments, the Notch is Notch 2, Notch 3, and Notch 4. In embodiments, the Notch is Notch 1, Notch 2, Notch 3, and Notch 4.

In an aspect is provided a method of decreasing the level of Notch (e.g., one or more of Notch 1, Notch 2, Notch 3, and/or Notch 4) activity in a cell, the method including contacting the cell with a compound described herein, or a salt (e.g., pharmaceutically acceptable salt) thereof. In embodiments, the compound is administered in an effective amount. In embodiments, the compound as described herein is included in an effective amount. In embodiments, the Notch is Notch 1. In embodiments, the Notch is Notch 2. In embodiments, the Notch is Notch 3. In embodiments, the Notch is Notch 4. In embodiments, the Notch is Notch 1 and Notch 2. In embodiments, the Notch is Notch 1 and Notch 3. In embodiments, the Notch is Notch 1 and Notch 4. In embodiments, the Notch is Notch 2 and Notch 3. In embodiments, the Notch is Notch 2 and Notch 4. In embodiments, the Notch is Notch 3 and Notch 4. In embodiments, the Notch is Notch 1, Notch 2, and Notch 3. In embodiments, the Notch is Notch 1, Notch 2, and Notch 4. In embodiments, the Notch is Notch 1, Notch 3, and Notch 4. In embodiments, the Notch is Notch 2, Notch 3, and Notch 4. In embodiments, the Notch is Notch 1, Notch 2, Notch 3, and Notch 4.

In an aspect is provided a method of decreasing the level of CSL-Notch (e.g., one or more of Notch 1, Notch 2, Notch 3, and/or Notch 4)-Mastermind complex activity in a subject, the method including administering a compound described herein, or a salt (e.g., pharmaceutically acceptable salt) thereof, to the subject. In embodiments, the compound is administered in an effective amount. In embodiments, the compound as described herein is included in an effective amount. In embodiments, the Notch is Notch 1. In embodiments, the Notch is Notch 2. In embodiments, the Notch is Notch 3. In embodiments, the Notch is Notch 4. In embodiments, the Notch is Notch 1 and Notch 2. In embodiments, the Notch is Notch 1 and Notch 3. In embodiments, the Notch is Notch 1 and Notch 4. In embodiments, the Notch is Notch 2 and Notch 3. In embodiments, the Notch is Notch 2 and Notch 4. In embodiments, the Notch is Notch 3 and Notch 4. In embodiments, the Notch is Notch 1, Notch 2, and Notch 3. In embodiments, the Notch is Notch 1, Notch 2, and Notch 4. In embodiments, the Notch is Notch 1, Notch 3, and Notch 4. In embodiments, the Notch is Notch 2, Notch 3, and Notch 4. In embodiments, the Notch is Notch 1, Notch 2, Notch 3, and Notch 4.

In an aspect is provided a method of decreasing the level of CSL-Notch (e.g., one or more of Notch 1, Notch 2, Notch 3, and/or Notch 4)-Mastermind complex activity in a cell, the method including contacting the cell with a compound described herein, or a salt (e.g., pharmaceutically acceptable salt) thereof. In embodiments, the compound is administered in an effective amount. In embodiments, the compound as described herein is included in an effective amount. In embodiments, the Notch is Notch 1. In embodiments, the Notch is Notch 2. In embodiments, the Notch is Notch 3. In embodiments, the Notch is Notch 4. In embodiments, the Notch is Notch 1 and Notch 2. In embodiments, the Notch is Notch 1 and Notch 3. In embodiments, the Notch is Notch 1 and Notch 4. In embodiments, the Notch is Notch 2 and Notch 3. In embodiments, the Notch is Notch 2 and Notch 4. In embodiments, the Notch is Notch 3 and Notch 4. In embodiments, the Notch is Notch 1, Notch 2, and Notch 3. In embodiments, the Notch is Notch 1, Notch 2, and Notch 4. In embodiments, the Notch is Notch 1, Notch 3, and Notch 4. In embodiments, the Notch is Notch 2, Notch 3, and Notch 4. In embodiments, the Notch is Notch 1, Notch 2, Notch 3, and Notch 4.

In embodiments, the compound contacts Notch (e.g., one or more of Notch 1, Notch 2, Notch 3, and/or Notch 4) protein. In embodiments, the compound contacts both Notch (e.g., one or more of Notch 1, Notch 2, Notch 3, and/or Notch 4) protein and CSL protein at the interface between the two proteins. In embodiments, the Notch is Notch 1. In embodiments, the Notch is Notch 2. In embodiments, the Notch is Notch 3. In embodiments, the Notch is Notch 4. In embodiments, the Notch is Notch 1 and Notch 2. In embodiments, the Notch is Notch 1 and Notch 3. In embodiments, the Notch is Notch 1 and Notch 4. In embodiments, the Notch is Notch 2 and Notch 3. In embodiments, the Notch is Notch 2 and Notch 4. In embodiments, the Notch is Notch 3 and Notch 4. In embodiments, the Notch is Notch 1, Notch 2, and Notch 3. In embodiments, the Notch is Notch 1, Notch 2, and Notch 4. In embodiments, the Notch is Notch 1, Notch 3, and Notch 4. In embodiments, the Notch is Notch 2, Notch 3, and Notch 4. In embodiments, the Notch is Notch 1, Notch 2, Notch 3, and Notch 4.

In embodiments, the compound reduces Mastermind binding to Notch (e.g., one or more of Notch 1, Notch 2, Notch 3, and/or Notch 4). In embodiments, the Notch is Notch 1. In embodiments, the Notch is Notch 2. In embodiments, the Notch is Notch 3. In embodiments, the Notch is Notch 4. In embodiments, the Notch is Notch 1 and Notch 2. In embodiments, the Notch is Notch 1 and Notch 3. In embodiments, the Notch is Notch 1 and Notch 4. In embodiments, the Notch is Notch 2 and Notch 3. In embodiments, the Notch is Notch 2 and Notch 4. In embodiments, the Notch is Notch 3 and Notch 4. In embodiments, the Notch is Notch 1, Notch 2, and Notch 3. In embodiments, the Notch is Notch 1, Notch 2, and Notch 4. In embodiments, the Notch is Notch 1, Notch 3, and Notch 4. In embodiments, the Notch is Notch 2, Notch 3, and Notch 4. In embodiments, the Notch is Notch 1, Notch 2, Notch 3, and Notch 4.

In embodiments, the compound reduces CSL binding to Notch (e.g., one or more of Notch 1, Notch 2, Notch 3, and/or Notch 4). In embodiments, the Notch is Notch 1. In embodiments, the Notch is Notch 2. In embodiments, the Notch is Notch 3. In embodiments, the Notch is Notch 4. In embodiments, the Notch is Notch 1 and Notch 2. In embodiments, the Notch is Notch 1 and Notch 3. In embodiments, the Notch is Notch 1 and Notch 4. In embodiments, the Notch is Notch 2 and Notch 3. In embodiments, the Notch is Notch 2 and Notch 4. In embodiments, the Notch is Notch 3 and Notch 4. In embodiments, the Notch is Notch 1, Notch 2, and Notch 3. In embodiments, the Notch is Notch 1, Notch 2, and Notch 4. In embodiments, the Notch is Notch 1, Notch 3, and Notch 4. In embodiments, the Notch is Notch 2, Notch 3, and Notch 4. In embodiments, the Notch is Notch 1, Notch 2, Notch 3, and Notch 4.

In an aspect is provided a method of inhibiting cancer growth in a subject in need thereof, the method including administering to the subject in need thereof an effective amount of a compound described herein, or a salt (e.g., pharmaceutically acceptable salt) thereof. In embodiments, the compound is administered in a therapeutically effective amount. In embodiments, the compound as described herein is included in an effective amount.

In an aspect is provided a method of treating a cancer in a subject in need thereof, the method including administering to the subject in need thereof an effective amount of a compound described herein, or a salt (e.g., pharmaceutically acceptable salt) thereof. In embodiments, the compound is administered in a therapeutically effective amount. In embodiments, the compound as described herein is included in an effective amount.

In embodiments, the cancer is breast cancer, esophageal cancer, leukemia, prostate cancer, colorectal cancer, lung cancer, central nervous system cancer. In embodiments, the cancer is T-cell acute lymphoblastic leukemia, B-cell acute lymphoblastic leukemia, chronic lymphocytic leukemia, myelomonocytic leukemia, breast cancer, medulloblastoma, colorectal cancer, non-small cell lung carcinoma, melanoma, cerebral autosomal-dominant ateriopathy with sub-cortical infarcts and leukoencephalophathy, hepatocellular carcinoma, pancreatic ductal adenocarcinoma, head and neck squamous cell carcinoma, renal cell adenocarcinoma, basal cell carcinoma, luminal A breast cancer, luminal B breast cancer, or fibrosarcoma.

In embodiments, the method further includes co-administering an anti-cancer agent to the subject in need. In embodiments, the anti-cancer agent is administered in a therapeutically effective amount.

In an aspect is provided a method of treating a disease associated with Notch (e.g., one or more of Notch 1, Notch 2, Notch 3, and/or Notch 4) activity in a subject in need thereof, the method including administering to the subject in need thereof an effective amount of a compound described herein, or a salt (e.g., pharmaceutically acceptable salt) thereof. In embodiments, the compound is administered in a therapeutically effective amount. In embodiments, the compound as described herein is included in an effective amount. In embodiments, the Notch is Notch 1. In embodiments, the Notch is Notch 2. In embodiments, the Notch is Notch 3. In embodiments, the Notch is Notch 4. In embodiments, the Notch is Notch 1 and Notch 2. In embodiments, the Notch is Notch 1 and Notch 3. In embodiments, the Notch is Notch 1 and Notch 4. In embodiments, the Notch is Notch 2 and Notch 3. In embodiments, the Notch is Notch 2 and Notch 4. In embodiments, the Notch is Notch 3 and Notch 4. In embodiments, the Notch is Notch 1, Notch 2, and Notch 3. In embodiments, the Notch is Notch 1, Notch 2, and Notch 4. In embodiments, the Notch is Notch 1, Notch 3, and Notch 4. In embodiments, the Notch is Notch 2, Notch 3, and Notch 4. In embodiments, the Notch is Notch 1, Notch 2, Notch 3, and Notch 4.

In embodiments, the disease is cancer. In embodiments, the disease is multiple sclerosis. In embodiments, the disease is Tetralogy of Fallot or Alagille syndrome or Hajdu-Cheney syndrome.

In embodiments, the compound reduces the level of Notch (e.g., one or more of Notch 1, Notch 2, Notch 3, and/or Notch 4) protein contacting a CSL protein (e.g., in a cell, in a subject, compared to a control such as absence of the compound under otherwise identical conditions). In embodiments, the compound reduces the level of Notch (e.g., one or more of Notch 1, Notch 2, Notch 3, and/or Notch 4) protein contacting a Mastermind protein (e.g., in a cell, in a subject, compared to a control such as absence of the compound under otherwise identical conditions).

### V. Embodiments

Embodiment P1. A compound having the formula:

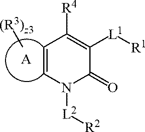

wherein,
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or , substituted or unsubstituted heteroalkylene;
R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R¹²)—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOn₃R^{3D}, —SOᵥ₃NR^{3A}R^{3B}, —NR3^{C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR3AC(O)R^{3C}, —NR3^{A}C(O)OR^{3C}, —NR3AOR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is independently an integer from 0 to 8;
R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D};
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴, are independently —F, —Cl, —Br, or —I;
n1, n2, and n3 are independently an integer from 0 to 4; and
m1, m2, m3, v1, v2, and v3 are independently 1 or 2;
or a salt thereof;
wherein the compound is not:

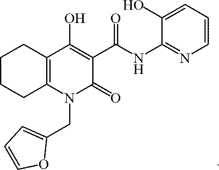

Embodiment P2. The compound of embodiment P1, having the formula:

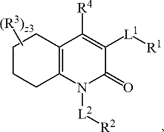

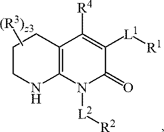

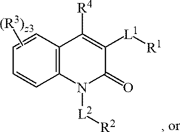

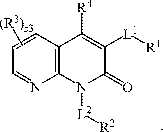

Embodiment P3. The compound of embodiment P1, having the formula:

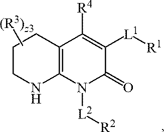

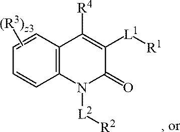

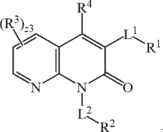

Embodiment P4. The compound of embodiment P1, having the formula:

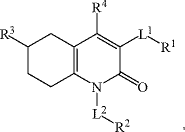

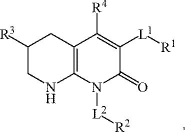

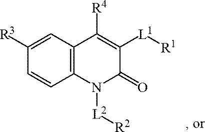

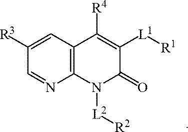

Embodiment P5. The compound of embodiment P1, having the formula:

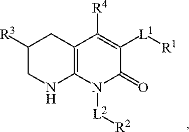

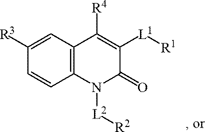

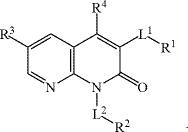

Embodiment P6. The compound of embodiment P1, having the formula:

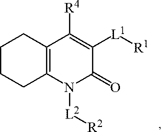

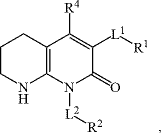

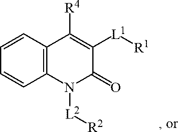

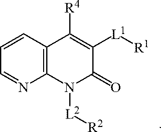

Embodiment P7. The compound of embodiment P1, having the formula:

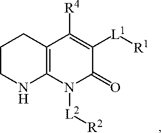

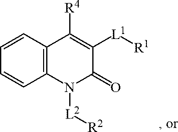

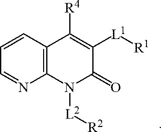

Embodiment P8. The compound of one of embodiments P1 to P5, wherein R³ is independently halogen, oxo, —CC1₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —NO₂, —SH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CH₃, —CH₂CH₃, —OCH₃, or —OCH₂CH₃.

Embodiment P9. The compound of one of embodiments P1 to P5, wherein R³ is independently —OH, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, —CH₃, —CH₂CH₃, —OCH₃, or —OCH₂CH₃.

Embodiment P10. The compound of one of embodiments P1 to P5, wherein R³ is independently —OCH₃.

Embodiment P11. The compound of one of embodiments P1 to P10, wherein R⁴ is independently —SR^{4D}, —NR^{4A}R^{4B}, or —OR^{4D};

R^{4A}, R^{4B}, and R^{4D} are independently hydrogen or unsubstituted C₁—C₆ alkyl; R^{4A} and R^{4B} substituents bonded to the same nitrogen atom may optionally be joined to form a substituted or unsubstituted 5 to 6 membered heterocycloalkyl;

Embodiment P12. The compound of one of embodiments P1 to P10, wherein R⁴ is independently —OR^{4D}; and R^{4D} is independently hydrogen or unsubstituted C₁—C₆ alkyl.

Embodiment P13. The compound of one of embodiments P1 to P10, wherein R⁴ is independently —OH.

Embodiment P14. The compound of one of embodiments P1 to P13, wherein L² is a bond or substituted or unsubstituted C₁—C₆ alkylene.

Embodiment P15. The compound of one of embodiments P1 to P13, wherein L² is a bond or unsubstituted C₁—C₄ alkylene.

Embodiment P16. The compound of one of embodiments P1 to P13, wherein L² is a bond.

Embodiment P17. The compound of one of embodiments P1 to P14, wherein L² is unsubstituted C₁—C₄ alkylene.

Embodiment P18. The compound of one of embodiments P1 to P14, wherein L² is unsubstituted methylene.

Embodiment P19. The compound of one of embodiments P1 to P18, wherein R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment P20. The compound of one of embodiments P1 to P18, wherein R² is independently unsubstituted alkyl.

Embodiment P21. The compound of one of embodiments P1 to P18, wherein R² is independently substituted or unsubstituted aryl or substituted or unsubstituted heteroaryl.

Embodiment P22. The compound of one of embodiments P1 to P18, wherein R² is independently substituted or unsubstituted phenyl or substituted or unsubstituted 5 to 6 membered heteroaryl.

Embodiment P23. The compound of one of embodiments P1 to P18, wherein R² is independently substituted or unsubstituted phenyl or substituted or unsubstituted 5 to 6 membered heteroaryl.

Embodiment P24. The compound of one of embodiments P1 to P18, wherein
R² is independently R²⁰-substituted phenyl or R²⁰-substituted 5 to 6 membered heteroaryl;
R²⁰ is independently halogen, —CX²⁰₃, —CHX²⁰₂, —CH₂X²⁰, —OCX²⁰₃, —OCH₂X²⁰, —OCHX²⁰₂, —CN, -SOₙ₂₀R^{20D}, —SO_{V20}NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R^{20B}, —N(O)ₘ₂₀, —NR^{20A}R^{20B}, —C(O)R^{20C}, —C(O)—OR^{20C}, —C(O)NR^{20A}R^{20B}, —OR^{20D}, —NR^{20A}SO₂R^{20D}, —NR^{20A}C(O)R^{20C}, —NR²⁰AC(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{20A,} R^{20B}, R^{20C}, and R^{20D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl;
X²⁰ is independently —F, —Cl, —Br, or —I;
n20 is independently an integer from 0 to 4; and
m20 and v20 are independently 1 or 2.

Embodiment P25. The compound of one of embodiments P1 to P18, wherein R² is independently R²⁰-substituted phenyl or R²⁰-substituted 5 to 6 membered heteroaryl; and R²⁰ is independently halogen.

Embodiment P26. The compound of one of embodiments P1 to P18, wherein R² is independently R²⁰-substituted phenyl or R²⁰-substituted 5 to 6 membered heteroaryl; and R²⁰ is independently —F.

Embodiment P27. The compound of one of embodiments P1 to P18, wherein R² is independently unsubstituted phenyl or unsubstituted 5 to 6 membered heteroaryl.

Embodiment P28. The compound of one of embodiments P1 to P27, wherein L¹ is —C(O)NH—, —NHC(O)—, —NHC(O)NH—, —SO₂NH—, —NHSO₂—, or substituted or unsubstituted heteroalkylene.

Embodiment P29. The compound of one of embodiments P1 to P27, wherein L¹ is —C(O)NH—, —NHC(O)—, —NHC(O)NH—, —SO₂NH—, —NHSO₂—, or substituted or unsubstituted 2 to 3 membered heteroalkylene.

Embodiment P30. The compound of one of embodiments P1 to P27, wherein
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, —N(R^{L1})CH₂—, —OCH₂—, —SCH₂—, —SO₂CH₂—, —C(O)CH₂—, —C(O)N(R^{L1})CH₂—, —N(R^{L1})C(O)CH₂—, —N(R^{L1})C(O)NHCH₂—, —NHC(O)N(R^{L1})CH₂—, —C(O)OCH₂—, —OC(O)CH₂—, —SO₂N(R^{L1})CH₂—, —N(R^{L1})SO₂CH₂—, —CH₂N(R^{L1})—, —CH₂O—, —CH₂S—, —CH₂SO₂—, —CH₂C(O)—, —CH₂C(O)N(R^{L1})—, —CH₂N(R^{L1})C(O)—, —CH₂N(R^{L1})C(O)NH—, —CH₂NHC(O)N(R^{L1})—, —CH₂C(O)O—, —CH₂OC(O)—, —CH₂SO₂N(R^{L1})—, —CH₂N(R^{L1})SO₂—; and
R^{L1} is independently hydrogen or unsubstituted C₁—C₄ alkyl.

Embodiment P31. The compound of one of embodiments P1 to P27, wherein L¹ is —C(O)NH—, —NHC(O)—, —NHC(O)NH—, —SO₂NH—, —NHSO₂—, —NHCH₂—,—CH₂NH—, —C(O)NHCH₂—, or —NHC(O)CH₂—.

Embodiment P32. The compound of one of embodiments P1 to P27, wherein
L¹ is —C(O)N(R^{L1})— or —C(O)N(R^{L1})CH₂—; and
R^{L1} is independently hydrogen or unsubstituted methyl.

Embodiment P33. The compound of one of embodiments P1 to P27, wherein L¹ is —C(O)NH—.

Embodiment P34. The compound of one of embodiments P1 to P27, wherein L¹ is —C(O)NH— wherein NH— is directly bonded to R¹.

Embodiment P35. The compound of one of embodiments P1 to P27, wherein L¹ is NHC(O)- wherein —C(O)— is directly bonded to R¹.

Embodiment P36. The compound of one of embodiments P1 to P27, wherein L¹ is —C(O)NHCH₂—.

Embodiment P37. The compound of one of embodiments P1 to P36, wherein; R¹ is independently substituted or unsubstituted C₁—C₆ alkyl, substituted or unsubstituted 2 to 6 membered heteroalkyl, substituted or unsubstituted C₃—C₆ cycloalkyl, substituted or unsubstituted 3 to 6 membered heterocycloalkyl, substituted or unsubstituted phenyl, or substituted or unsubstituted 5 to 6 membered heteroaryl.

Embodiment P38. The compound of one of embodiments P1 to P36, wherein;
R¹ is independently R¹⁰-substituted or unsubstituted C₁—C₆ alkyl, R¹⁰-substituted or unsubstituted 2 to 6 membered heteroalkyl, R¹⁰-substituted or unsubstituted C₃—C₆ cycloalkyl, R¹⁰-substituted or unsubstituted 3 to 6 membered heterocycloalkyl, R¹⁰-substituted or unsubstituted phenyl, or R¹⁰-substituted or unsubstituted 5 to 6 membered heteroaryl;
R¹⁰ is independently halogen, oxo, —CX¹⁰₃, —CHX¹⁰₂, —CH₂X¹⁰, —OCX¹⁰₃, —OCH₂X¹⁰, —OCHX¹⁰2, —CN, —SOₙ₁₀R^{10D}, —SOᵥ₁₀NR^{10A}R^{10B}, —NR^{1OC}NR^{1OA}R^{1OB}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10A,} R^{10B}, R^{10C}, and R^{10D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl;
X¹⁰ is independently —F, —Cl, —Br, or —I;
n10 is independently an integer from 0 to 4; and
m10 and v10 are independently 1 or 2.

Embodiment P39. The compound of one of embodiments P1 to P36, wherein;
R¹ is independently R¹⁰-substituted or unsubstituted C₃—C₆ cycloalkyl, R¹⁰-substituted or unsubstituted 3 to 6 membered heterocycloalkyl, R¹⁰-substituted or unsubstituted phenyl, or R¹⁰-substituted or unsubstituted 5 to 6 membered heteroaryl;
R¹⁰ is independently halogen, oxo, —CX¹⁰₃, —CHX¹⁰₂, —CH₂X¹⁰, —OCX¹⁰₃, —OCH₂X¹⁰, —OCHX¹⁰2, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR¹⁰AC(O)OR^{10C}, —NR^{10A}OR^{10C}, —SF₅, —N₃, substituted or unsubstituted C₁—C₆ alkyl, substituted or unsubstituted 2 to 6 membered heteroalkyl, substituted or unsubstituted C₃—C₆ cycloalkyl, substituted or unsubstituted 3 to 6 membered heterocycloalkyl, substituted or unsubstituted phenyl, or substituted or unsubstituted 5 to 6 membered heteroaryl;
R^{10A}, R^{10B}, R^{10C}, and R^{10D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted C₁—C₆ alkyl, substituted or unsubstituted 2 to 6 membered heteroalkyl, substituted or unsubstituted C₃—C₆ cycloalkyl, substituted or unsubstituted 3 to 6 membered heterocycloalkyl, substituted or unsubstituted phenyl, or substituted or unsubstituted 5 to 6 membered heteroaryl; R^{10A} and R^{10B} substituents bonded to the same nitrogen atom may optionally be joined to form a substituted or unsubstituted 3 to 6 membered heterocycloalkyl or substituted or unsubstituted 5 to 6 membered heteroaryl;
X¹⁰ is independently —F, —Cl, —Br, or —I;
n10 is independently an integer from 0 to 4; and
m10 and v10 are independently 1 or 2.

Embodiment P40. The compound of one of embodiments P1 to P36, wherein;
R¹ is independently R¹⁰-substituted or unsubstituted C₃—C₆ cycloalkyl, R¹⁰-substituted or unsubstituted 3 to 6 membered heterocycloalkyl, R¹⁰-substituted or unsubstituted phenyl, or R¹⁰-substituted or unsubstituted 5 to 6 membered heteroaryl;
R¹⁰ is independently halogen, —CX¹⁰₃, —CHX¹⁰₂, —CH₂X¹⁰, —OCX¹⁰₃, —OCH₂X¹⁰, —OCHX¹⁰2, —SR^{10D}, —OR^{10D}, unsubstituted C₁—C₄ alkyl, unsubstituted 2 to 4 membered heteroalkyl, unsubstituted C₃—C₆ cycloalkyl, unsubstituted 3 to 6 membered heterocycloalkyl, unsubstituted phenyl, or unsubstituted 5 to 6 membered heteroaryl;
R^{10D} is independently hydrogen or unsubstituted C₁—C₄ alkyl; and
X¹⁰ is independently —F, —Cl, —Br, or —I.

Embodiment P41. The compound of one of embodiments P1 to P36, wherein;
R¹ is independently R¹⁰-substituted or unsubstituted C₃—C₆ cycloalkyl, R¹⁰-substituted or unsubstituted 3 to 6 membered heterocycloalkyl, R¹⁰-substituted or unsubstituted phenyl, or R¹⁰-substituted or unsubstituted 5 to 6 membered heteroaryl; and
R¹⁰ is independently halogen, —OH, —OCH₃, —CH₃, unsubstituted 6 membered heterocycloalkyl.

Embodiment P42. The compound of one of embodiments P1 to P36, wherein; R¹ is independently

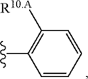

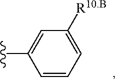

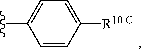

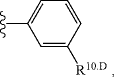

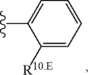

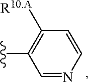

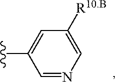

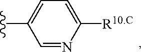

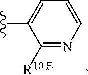

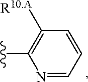

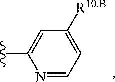

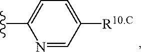

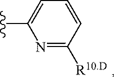

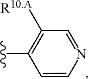

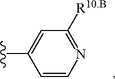

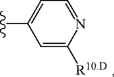

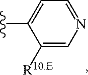

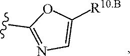

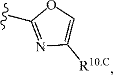

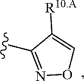

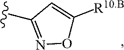

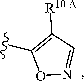

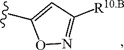

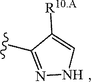

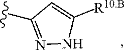

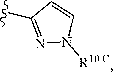

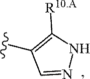

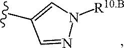

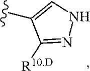

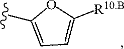

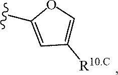

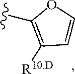

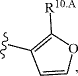

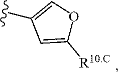

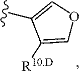

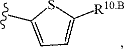

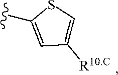

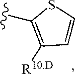

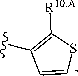

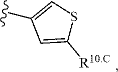

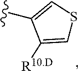

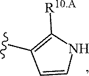

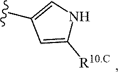

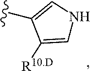

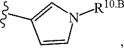

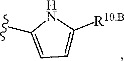

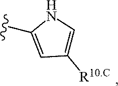

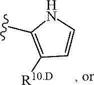

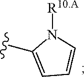

R^{10.A}, R^{10.B}, R^{10.C}, R^{10.D}, and R^{10.E} are independently —F, —C1, —CH₃, —OCH₃, —OH, unsubstituted morpholinyl, or unsubstituted piperazinyl.

Embodiment P43. The compound of one of embodiments P1 to P36, wherein; R¹ is independently

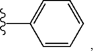

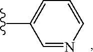

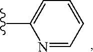

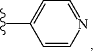

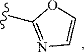

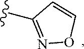

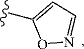

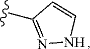

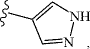

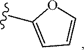

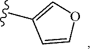

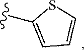

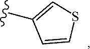

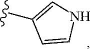

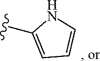

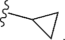

Embodiment P44. A pharmaceutical composition comprising the compound of any one of embodiments P1 to P43 and a pharmaceutically acceptable excipient.

Embodiment P45. A method of decreasing the level of Notch protein activity in a subject, said method comprising administering a compound of one of embodiments P1 to P43 to said subject.

Embodiment P46. A method of decreasing the level of Notch activity in a cell, said method comprising contacting said cell with a compound of one of embodiments P1 to P43.

Embodiment P47. A method of decreasing the level of CSL-Notch-Mastermind complex activity in a subject, said method comprising administering a compound of one of embodiments P1 to P43 to said subject.

Embodiment P48. A method of decreasing the level of CSL-Notch-Mastermind complex activity in a cell, said method comprising contacting said cell with a compound of one of embodiments P 1 to P43.

Embodiment P49. The method of one of embodiments P45 to P48, wherein the compound contacts Notch protein.

Embodiment P50. The method of one of embodiments P45 to P49, wherein the compound reduces Mastermind binding to Notch.

Embodiment P51. The method of one of embodiments P45 to P50, wherein the compound reduces CSL binding to Notch.

Embodiment P52. A method of inhibiting cancer growth in a subject in need thereof, said method comprising administering to the subject in need thereof an effective amount of a compound of one of embodiments P 1 to P43.

Embodiment P53. A method of treating a cancer in a subject in need thereof, said method comprising administering to the subject in need thereof an effective amount of a compound of one of embodiments P 1 to P43.

Embodiment P54. The method of embodiment P53, wherein the cancer is breast cancer, esophageal cancer, leukemia, prostate cancer, colorectal cancer, lung cancer, central nervous system cancer.

Embodiment P55. The method of one of embodiments P53 to P54, further comprising co-administering an anti-cancer agent to said subject in need.

Embodiment P56. A pharmaceutical composition comprising a pharmaceutically acceptable excipient and a compound having the formula:

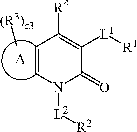

wherein,
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or , substituted or unsubstituted heteroalkylene;
R¹ is independently hydrogen, halogen, —CX¹₃, —CHX¹₂, —CH₂X¹, —OCX¹₃, —OCH₂X¹, —OCHX12, —CN, —SOniR^{1D}, —SOᵥ₁NR^{1A}R^{1B}, —NR^{1C}NR^{1A}R^{1B}, —ONR^{1A}R^{1B}, —NHC(O)NR1^{C}NR1^{A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR1^{A}R^{1B}, —OR ^{1D}, —NR^{1A}SO₂R^{1D}, —NR1AC(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R¹²)—, —O—, —S—, —SO₂—, —C(O)—, —C(O)N(R^{L2})—, —N(R12)C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is independently hydrogen, halogen, —CX²₃, —CHX²₂, —CH₂X², —OCX²₃, —OCH₂X², —OCHX²₂, —CN, —S0n2R2D, —SO_{V2}NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOn₃R^{3D}, —SOᵥ₃NR^{3A}R^{3B}, —NR3^{C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is independently an integer from 0 to 8;
R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OW^{D} ;
R^{1A}_{>} R^{1B}_{>} R^{1C} , R^{1D >} R2^{A}_{>} R2^{B}_{>} R2^{C >} R2^{D >} R^{3A}_{>} R^{3B} , R^{3C}_{>} R^{3D}_{>} R4^{A ,} R^{4B >} R4^{D >} R^{Ll >}_{and} R^{L2}ₐᵣₑ independently hydrogen, —CCl₃, —CBr₃, —CF₃, —CI₃, —CHC12, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHC12, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴, are independently —F, —C1, —Br, or —I;
n1, n2, and n3 are independently an integer from 0 to 4; and
m1, m2, m3, v1, v2, and v3 are independently 1 or 2;
or a salt thereof.

Embodiment P57. A method of decreasing the level of Notch protein activity in a subject or decreasing the level of CSL-Notch-Mastermind complex activity in a subject, said method comprising administering to said subject, a compound having the formula:

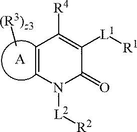

wherein,
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or , substituted or unsubstituted heteroalkylene;
R¹ is independently hydrogen, halogen, —CX¹₃, —CHX¹₂, —CH₂X¹, —OCX¹₃, —OCH₂X¹, —OCHX12, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR1^{C}NR1^{A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR1^{A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R¹²)—, —O—, —S—, —SO₂—, —C(O)—, —C(O)N(R^{L2})—, —N(R12)C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is independently hydrogen, halogen, —CX²₃, —CHX²₂, —CH₂X², —OCX²₃, —OCH₂X², —OCHX²₂, —CN, —S0n2R2D, —SO_{V2}NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOn₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is independently an integer from 0 to 8;
R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D};
R^{1A}, R^{1B}, R^{1C} , R^{1D >} R^{2A}, R^{2B}, R^{2C}, R^{2D}, R^{3A}, R^{3C}, R^{3C}, R^{3D}, R^{4A}, R^{4B}, R^{4D}, R^{L1}, _{and} R^{L2}ₐᵣₑ independently hydrogen, —CCl₃, —CBr₃, —CF₃, —CI₃, —CHC12, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHC12, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴, are independently —F, —Cl, —Br, or —I;
n1, n2, and n3 are independently an integer from 0 to 4; and
ml, m2, m3, v1, v2, and v3 are independently 1 or 2;
or a salt thereof.

Embodiment P58. A method of decreasing the level of Notch activity in a cell or decreasing the level of CSL-Notch-Mastermind complex activity in a cell, said method comprising contacting said cell with a compound having the formula:

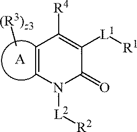

wherein,
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or , substituted or unsubstituted heteroalkylene;
R¹ is independently hydrogen, halogen, —CX¹₃, —CHX¹₂, —CH₂X¹, —OCX¹₃, —OCH₂X¹, —OCHX12, —, —SOₙ₁R^{1D}, —SOᵥ₁NR^{1A}R^{1B}, —NR^{1C}NR^{1A}R^{1B}, —ONR^{1A}R^{1B}, —NHC(O)NR1^{C}NR1^{A}R^{1B}, —NHC(O)NR^{1A}R^{1B}, —N(O)ml, —NR^{1A}R^{1B}, —C(O)R^{1C}, —C(O)—OR^{1c}, —C(O)NR1^{A}R^{1B}, —OR ^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R¹²)—, —O—, —S—, —SO₂—, —C(O)—, —C(O)N(R¹²)—, —N(R12)C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is independently hydrogen, halogen, —CX²₃, —CHX²₂, —CH₂X², —OCX²₃, —OCH₂X², —OCHX²₂, —CN, —S0n2R2D, —SO_{V2}NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOn₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R3^{B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is independently an integer from 0 to 8;
R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D};
R^{1A}, R^{1B}, R^{1C}, R^{1D}, R^{2A}_{,} R^{2B}, R^{2C}, R^{2D}, R^{3A}_{,} R^{3B}, R^{3C}_{,} R^{3D}_{,} R^{4A}, R^{4B}, R^{4D}, R^{L1}, and R^{L2} are independently hydrogen, —CCl₃, —CBr₃, —CF₃, —CI₃, —CHC1₂, —CHBr₂, —CHF₂, —CHI₂, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHC12, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴, are independently —F, —Cl, —Br, or —I;
n1, n2, and n3 are independently an integer from 0 to 4; and
ml, m2, m3, v1, v2, and v3 are independently 1 or 2;
or a salt thereof.

Embodiment P59. The method of one of embodiments P57 to P58, wherein the compound contacts Notch protein.

Embodiment P60. The method of one of embodiments P57 to P59, wherein the compound reduces Mastermind binding to Notch.

Embodiment P61. The method of one of embodiments P57 to P60, wherein the compound reduces CSL binding to Notch.

Embodiment P62. A method of inhibiting cancer growth in a subject in need thereof or treating a cancer in a subject in need thereof, said method comprising administering to the subject in need thereof an effective amount of a compound having the formula:

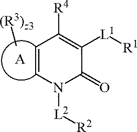

wherein,
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NH C(O)N(R^{L1})—, —C(O)O—, —OC(O)—, —SO₂N(R^{L1})—, —N(R^{L1})SO₂—, substituted or unsubstituted alkylene, or , substituted or unsubstituted heteroalkylene;
R¹ is independently hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is independently hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is independently an integer from 0 to 8;
R⁴ is independently hydrogen, halogen, —CX⁴₃, —CHX⁴₂, —CH₂X⁴, —OCX⁴₃, —OCH₂X⁴, —OCHX⁴₂, —CN, —SR^{4D}, -NR^{4A}R^{4B}, or —OR^{4D};
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴, are independently —F, —Cl, —Br, or —I;
n1, n2, and n3 are independently an integer from 0 to 4; and
m1, m2, m3, v1, v2, and v3 are independently 1 or 2;
or a salt thereof.

Embodiment P63. The method of embodiment P62, wherein the cancer is breast cancer, esophageal cancer, leukemia, prostate cancer, colorectal cancer, lung cancer, central nervous system cancer.

Embodiment P64. The method of one of embodiments P62 to P63, further comprising co-administering an anti-cancer agent to said subject in need.

### VI. Additional Embodiments

Embodiment 1. A compound having the formula:

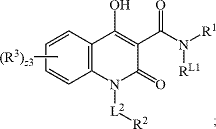

wherein
R¹ is

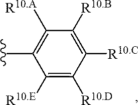

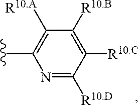

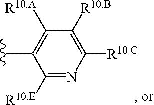

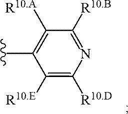

R^{10.A} is hydrogen, halogen, —CX^{10.A}₃, —CHX^{10.A}₂, —CH₂X^{10.A}, —OCX^{10.A}₃, —OCH₂X^{10.A}, —OCHX^{10.A}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10.B} is hydrogen, halogen, —CX^{10.B}₃, —CHX^{10.B}₂, —CH₂X^{10.B}, —OCX^{10.B}₃, —OCH₂X^{10.B}, —OCHX^{10.B}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10.C} is hydrogen, halogen, —CX^{10.C}₃, —CHX^{10.C}₂, —CH₂X^{10.C}, —OCX^{10.C}₃, —OCH₂X^{10.C}, —OCHX^{10.C}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10.D} is hydrogen, halogen, —CX^{10.D}₃, —CHX^{10.D}₂, —CH₂X^{10.D}, —OCX^{10.D}₃, —OCH₂X^{10D}, —OCHX^{10.D}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10.E} is hydrogen, halogen, —CX^{10.E}₃, —CHX^{10.E}₂, —CH₂X^{10.E}, —OCX^{10.E}₃, —OCH₂X^{10.E}, —OCHX^{10.E}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 4;
R^{2A}, R^{2B}, R^{2C}, R^{2D}, R^{3A}, R^{3B}, R^{3C}, R^{3D}, R^{10A}, R^{10B}, R^{10C}, R^{10D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl;
X², X³, X^{10.A}, X^{10.B}, X^{10.C}, X^{10.D}, and X^{10.E} are independently —F, —Cl, —Br, or —I;
n2, n3, and n10 are independently an integer from 0 to 4; and
m2, m3, m10, v2, v3, and v10 are independently 1 or 2;
or a pharmaceutically acceptable salt thereof;
wherein —L²—R² is not hydrogen; and
wherein at least one of R^{10.A}, R^{10.B} , R^{10.C} , R^{10.D}, or R¹⁰^{.}^{E} is a substituted or unsubstituted cycloalkyl or substituted or unsubstituted heterocycloalkyl.

Embodiment 2. The compound of embodiment 1, wherein R¹ is

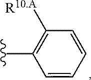

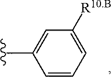

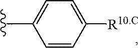

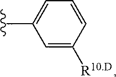

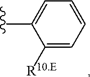

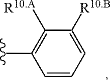

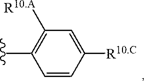

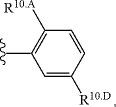

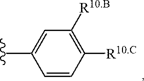

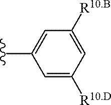

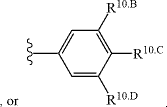

Embodiment 3. The compound of one of embodiments 1 to 2, wherein R^{10.A}, R^{10.B}, R^{10.C}, R^{10.D}, or R^{10.E} is independently halogen, substituted or unsubstituted C₆ cycloalkyl, or substituted or unsubstituted 6 membered heterocycloalkyl.

Embodiment 4. The compound of one of embodiments 1 to 2, wherein R^{10.A}, R^{10.B}, R^{10.C}, R^{10.D}, or R^{10.E} is independently a substituted or unsubstituted 6 membered heterocycloalkyl.

Embodiment 5. The compound of one of embodiments 1 to 2, wherein R^{10.A}, R^{10.B}, R^{10.C}, R^{10.D}, or R^{10.E} is independently a substituted or unsubstituted morpholinyl or substituted or unsubstituted piperazinyl.

Embodiment 6. The compound of one of embodiments 1 to 2, wherein R^{10.A}, R^{10.B}, R^{10.C}, R^{10.D}, or R^{10.E} is independently

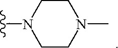

Embodiment 7. The compound of embodiment 1, wherein R¹ is

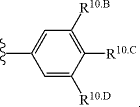

Embodiment 8. The compound of embodiment 7, wherein R^{10.B} and R^{10.D} are independently halogen, and R^{10.C} is substituted or unsubstituted C₆ cycloalkyl or substituted or unsubstituted 6 membered heterocycloalkyl.

Embodiment 9. The compound of embodiment 1, wherein R¹ is

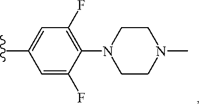

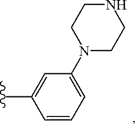

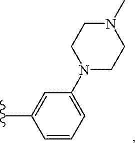

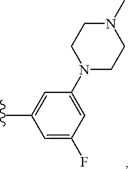

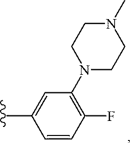

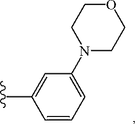

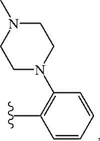

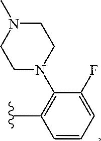

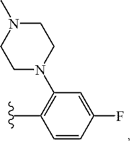

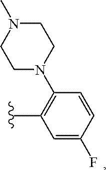

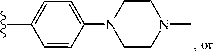

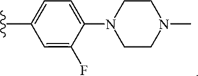

Embodiment 10. The compound of one of embodiments 1 to 9, wherein R^{L1} is hydrogen, unsubstituted methyl, unsubstituted ethyl, unsubstituted isopropyl, or unsubstituted cyclopropyl.

Embodiment 11. The compound of one of embodiments 1 to 9, wherein R^{L1} is hydrogen.

Embodiment 12. The compound of one of embodiments 1 to 11, wherein z3 is 0.

Embodiment 13. The compound of one of embodiments 1 to 11, wherein R³ is independently halogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —NO₂, —SH, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, —CH₃, —CH₂CH₃, —OCH₃, —OCH₂CH₃, or substituted or unsubstituted 3 to 6 membered heterocycloalkyl.

Embodiment 14. The compound of one of embodiments 1 to 11, wherein R³ is independently substituted or unsubstituted 3 to 6 membered heterocycloalkyl.

Embodiment 15. The compound of one of embodiments 1 to 11, wherein R³ is independently substituted or unsubstituted morpholinyl or substituted or unsubstituted piperazinyl.

Embodiment 16. The compound of one of embodiments 1 to 11, wherein R³ is independently —Br, —OCH₃, or substituted or unsubstituted piperazinyl.

Embodiment 17. The compound of one of embodiments 1 to 16, wherein L² is a bond or substituted or unsubstituted C₁—C₆ alkylene.

Embodiment 18. The compound of one of embodiments 1 to 16, wherein L² is a bond or unsubstituted C₁—C₄ alkylene.

Embodiment 19. The compound of one of embodiments 1 to 16, wherein L² is a bond.

Embodiment 20. The compound of one of embodiments 1 to 16, wherein L² is unsubstituted C₁—C₄ alkylene.

Embodiment 21. The compound of one of embodiments 1 to 16, wherein L² is unsubstituted methylene.

Embodiment 22. The compound of one of embodiments 1 to 21, wherein R² is hydrogen, halogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Embodiment 23. The compound of one of embodiments 1 to 21, wherein R² is unsubstituted alkyl.

Embodiment 24. The compound of one of embodiments 1 to 21, wherein R² is unsubstituted C₁—C₄ alkyl.

Embodiment 25. The compound of one of embodiments 1 to 21, wherein R² is unsubstituted isobutyl.

Embodiment 26. The compound of one of embodiments 1 to 21, wherein R² is substituted or unsubstituted aryl or substituted or unsubstituted heteroaryl.

Embodiment 27. The compound of one of embodiments 1 to 21, wherein R² is substituted or unsubstituted phenyl or substituted or unsubstituted 5 to 6 membered heteroaryl.

Embodiment 28. The compound of one of embodiments 1 to 21, wherein
R² is R²⁰-substituted phenyl or R²⁰-substituted 5 to 6 membered heteroaryl;
R²⁰ is independently halogen, —CX²⁰₃, —CHX²⁰₂, —CH₂X²⁰, -OCX²⁰₃, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R^{20B}, —N(O)ₘ₂₀, —NR^{20A}R^{20B}, —C(O)R^{20C}, —C(O)—OR^{20C}, —C(O)NR^{20A}R^{20B}, —OR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{20A,} R^{20B}, R^{20C}, and R^{20D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl;
X²⁰ is independently —F, —Cl, —Br, or —I;
n20 is an integer from 0 to 4; and
m20 and v20 are independently 1 or 2.

Embodiment 29. The compound of one of embodiments 1 to 21, wherein
R² is R²⁰-substituted phenyl or R²⁰-substituted 5 to 6 membered heteroaryl; and
R²⁰ is independently halogen.

Embodiment 30. The compound of one of embodiments 1 to 21, wherein
R² is R²⁰-substituted phenyl or R²⁰-substituted 5 to 6 membered heteroaryl; and
R²⁰ is independently —F.

Embodiment 31. The compound of one of embodiments 1 to 21, wherein R² is unsubstituted phenyl or unsubstituted 5 to 6 membered heteroaryl.

Embodiment 32. A compound having the formula:

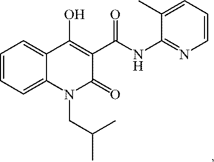

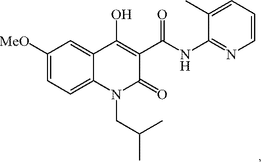

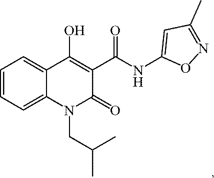

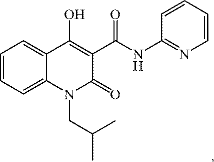

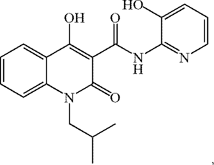

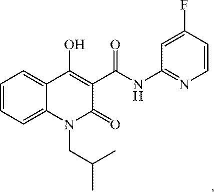

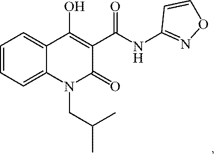

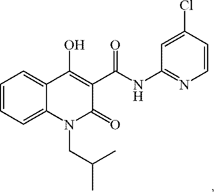

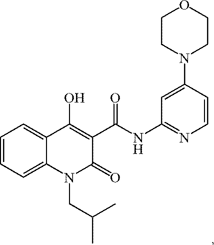

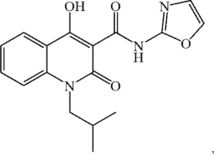

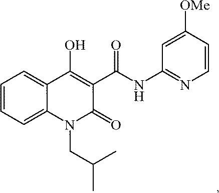

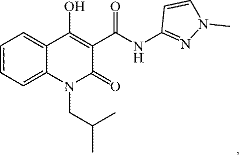

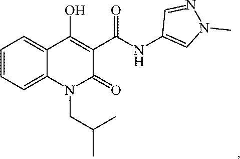

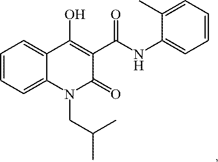

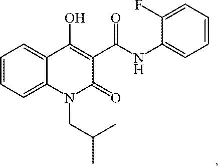

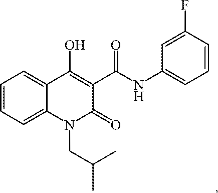

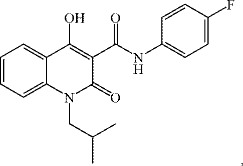

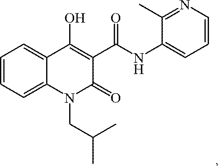

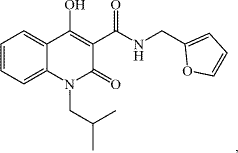

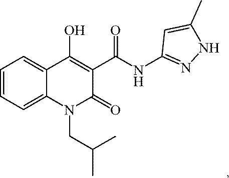

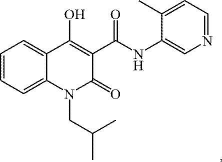

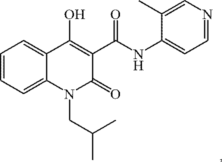

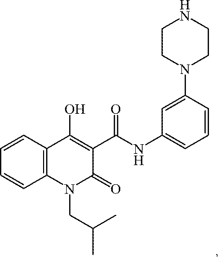

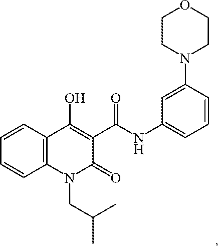

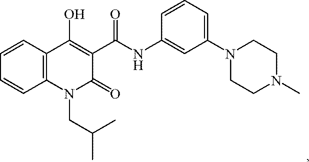

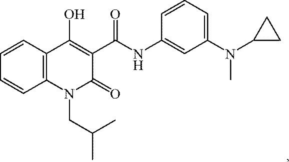

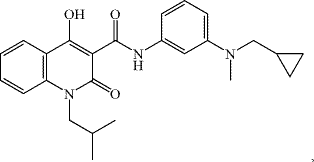

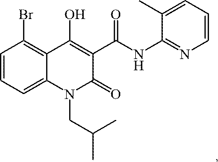

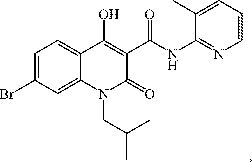

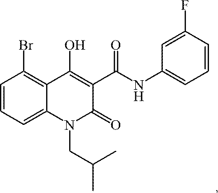

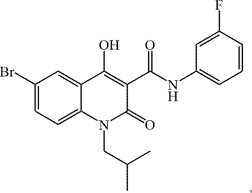

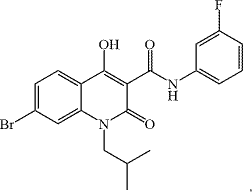

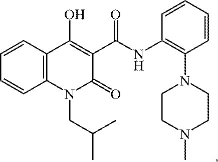

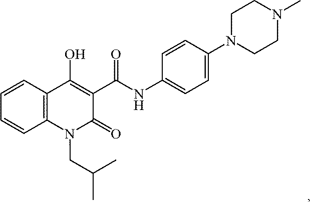

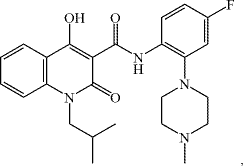

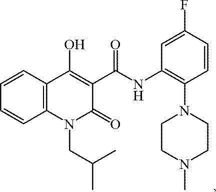

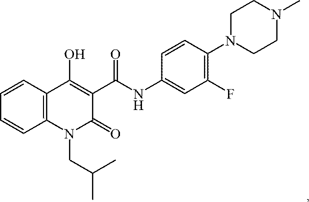

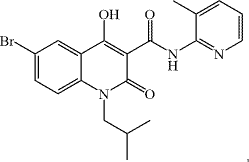

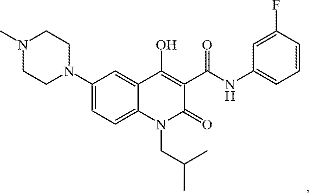

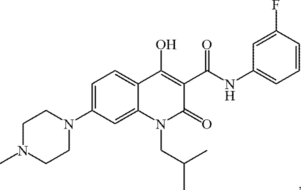

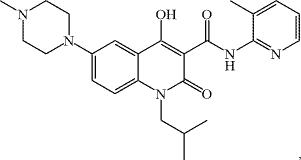

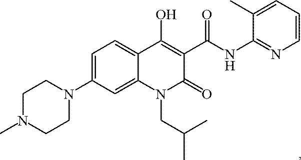

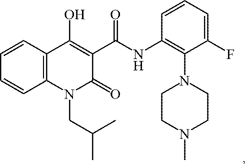

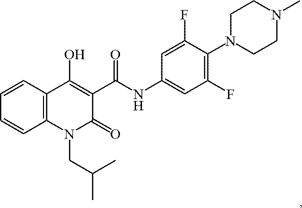

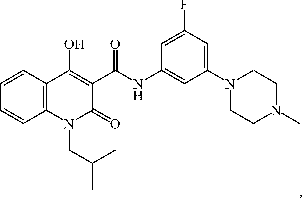

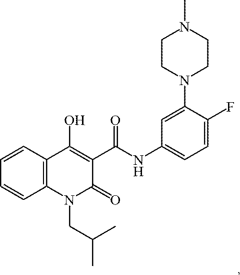

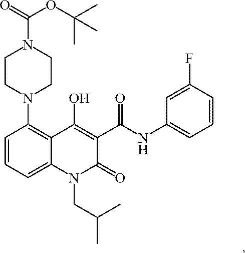

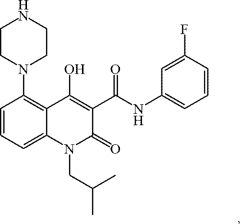

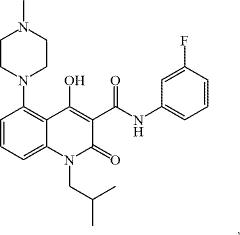

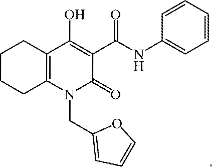

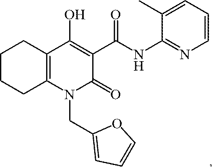

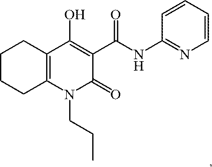

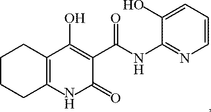

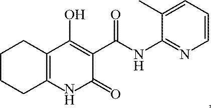

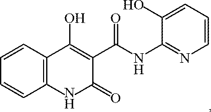

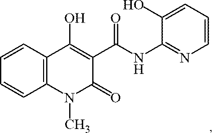

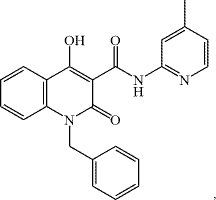

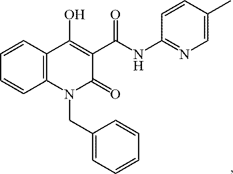

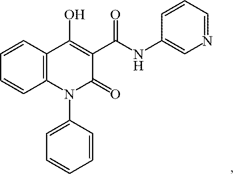

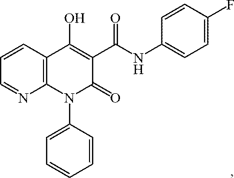

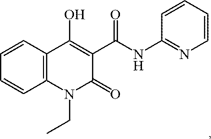

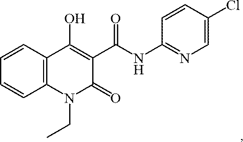

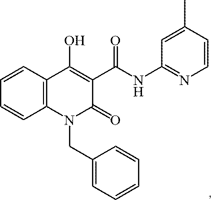

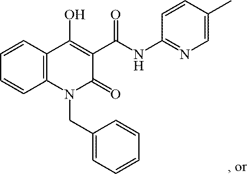

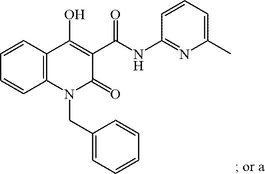

pharmaceutically acceptable salt thereof.

Embodiment 33. A pharmaceutical composition comprising the compound of one of embodiments 1 to 32 and a pharmaceutically acceptable excipient.

Embodiment 34. A method of decreasing the level of Notch protein activity in a subject, said method comprising administering a compound of one of embodiments 1 to 32 to said subject.

Embodiment 35. A method of decreasing the level of Notch activity in a cell, said method comprising contacting said cell with a compound of one of embodiments 1 to 32.

Embodiment 36. A method of decreasing the level of CSL-Notch-Mastermind complex activity in a subject, said method comprising administering a compound of one of embodiments 1 to 32 to said subject.

Embodiment 37. A method of decreasing the level of CSL-Notch-Mastermind complex activity in a cell, said method comprising contacting said cell with a compound of one of embodiments 1 to 32.

Embodiment 38. The method of one of embodiments 34 to 37, wherein the compound contacts Notch protein.

Embodiment 39. The method of one of embodiments 34 to 38, wherein the compound reduces Mastermind binding to Notch.

Embodiment 40. The method of one of embodiments 34 to 39, wherein the compound reduces CSL binding to Notch.

Embodiment 41. A method of inhibiting cancer growth in a subject in need thereof, said method comprising administering to the subject in need thereof an effective amount of a compound of one of embodiments 1 to 32.

Embodiment 42. A method of treating a cancer in a subject in need thereof, said method comprising administering to the subject in need thereof an effective amount of a compound of one of embodiments 1 to 32.

Embodiment 43. The method of embodiment 42, wherein the cancer is breast cancer, esophageal cancer, leukemia, prostate cancer, colorectal cancer, lung cancer, central nervous system cancer.

Embodiment 44. The method of one of embodiments 42 to 43, further comprising co-administering an anti-cancer agent to said subject in need.

Embodiment 45. A pharmaceutical composition comprising a pharmaceutically acceptable excipient and a compound having the formula:

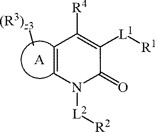

wherein
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or, substituted or unsubstituted heteroalkylene;
R¹ is hydrogen, halogen, —CX¹₃, —CHX¹₂, -CH₂X¹, —OCX¹₃, —OCH₂X¹, -OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 8;
R⁴ is hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D};
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴ are independently —F, —Cl, —Br, or —I;
n1, n2, and n3 are independently an integer from 0 to 4; and
m1, m2, m3, v1, v2, and v3 are independently 1 or 2;
or a salt thereof.

Embodiment 46. A method of decreasing the level of Notch protein activity in a subject or decreasing the level of CSL-Notch-Mastermind complex activity in a subject, said method comprising administering to said subject, a compound having the formula:

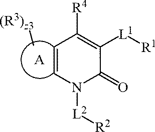

wherein
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or, substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, -ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, -NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 8;
R⁴ is hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D};
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴ are independently —F, —Cl, —Br, or —I;
n1, n2, and n3 are independently an integer from 0 to 4; and
m1, m2, m3, v1, v2, and v3 are independently 1 or 2;
or a salt thereof.

Embodiment 47. A method of decreasing the level of Notch activity in a cell or decreasing the level of CSL-Notch-Mastermind complex activity in a cell, said method comprising contacting said cell with a compound having the formula:

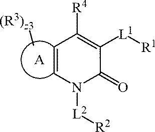

wherein,
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or, substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 8;
R⁴ is hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D};
R^{1A}, R^{1B}, R^{1C}, R^{1D}, R^{2A}, R^{2B}, R^{2C}, R^{2D}, R^{3A}, R^{3B}, R^{3C}, R^{3D}, R^{4A}, R^{4B}, R^{4D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴ are independently —F, —Cl, —Br, or —I;
n1, n2, and n3 are independently an integer from 0 to 4; and
m1, m2, m3, v1, v2, and v3 are independently 1 or 2;
or a salt thereof.

Embodiment 48. The method of one of embodiments 46 to 47, wherein the compound contacts Notch protein.

Embodiment 49. The method of one of embodiments 46 to 48, wherein the compound reduces Mastermind binding to Notch.

Embodiment 50. The method of one of embodiments 46 to 49, wherein the compound reduces CSL binding to Notch.

Embodiment 51. A method of inhibiting cancer growth in a subject in need thereof or treating a cancer in a subject in need thereof, said method comprising administering to the subject in need thereof an effective amount of a compound having the formula:

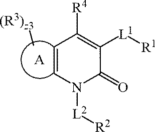

wherein
L¹ is a bond, —N(R^{L1})—, —O—, —S—, —SO₂—, —C(O)—, —C(O)N(R^{L1})—, —N(R^{L1})C(O)—, —N(R^{L}′)C(O)NH—, —NHC(O)N(R^{L1})—, —C(O)O—, —OC(O)—, —SO₂N(R^{L1})—, —N(R^{L}1)SO₂—, substituted or unsubstituted alkylene, or , substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR1^{A}R^{1B}, —OR^{1D}, —NR^{1A}SO₂R^{1D}, —NR1AC(O)R^{1C}, —NR1AC(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R¹²)—, —O—, —S—, —SO₂—, —C(O)—, —C(O)N(R^{L2})—, —N(R12)C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², —OCHX²₂, —CN, —SOₙ₂R^{2D}, —SO_{V2}NR^{2A}R^{2B}, —NR^{2C}NR^{2A}R^{2B}, —ONR^{2A}R^{2B}, —NHC(O)NR2^{C}NR2^{A}R2^{B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR2AC(O)R^{2C}, —NR2AC(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR3^{C}NR3^{A}R3^{B}, —NHC(O)NR3^{A}R^{3B}, —N(O)m₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR3AC(O)R^{3C}, —NR3AC(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 8;
R⁴ is hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OW^{D};
R^{1A}_{>} R^{1B}_{>} R^{1C} , R^{1D >} R2^{A}_{>} R2^{B}_{>} R2^{C}_{>} R2^{D >} R^{3A}_{>} R^{3B} , R^{3C}_{>} R^{3D}_{>} R4^{A ,} R^{4B >} R4^{D >} R^{Ll >}_{and} R^{L2}ₐᵣₑ independently hydrogen, —CCl₃, —CBr₃, —CF₃, —CI₃, —CHC1₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHC12, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴ are independently —F, —Cl, —Br, or —I;
n1, n2, and n3 are independently an integer from 0 to 4; and
ml, m2, m3, v1, v2, and v3 are independently 1 or 2;
or a salt thereof.

Embodiment 52. The method of embodiment 51, wherein the cancer is breast cancer, esophageal cancer, leukemia, prostate cancer, colorectal cancer, lung cancer, central nervous system cancer.

Embodiment 53. The method of one of embodiments 51 to 52, further comprising co-administering an anti-cancer agent to said subject in need.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1: Experimental Procedures and Characterization Data

SSTN-513

Synthesis of 4-hydroxy-1-isobutyl-N-(3-methylpyridin-2-yl)-2-oxo-1,2-dihydroquinoline-3-carboxamide (SSTN-513)

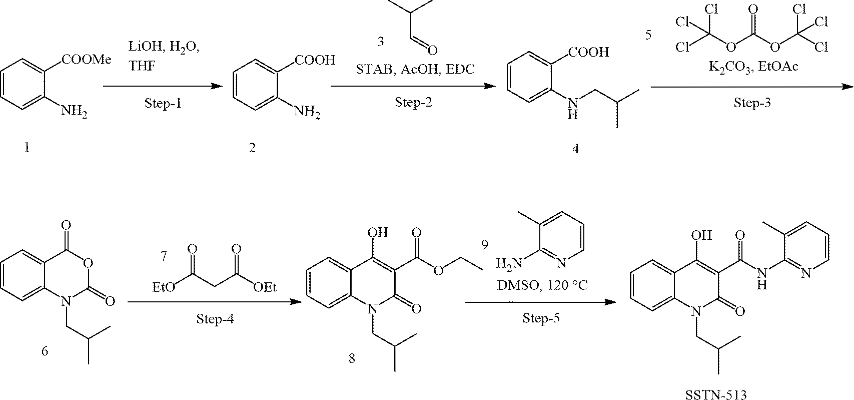

### Step-1: 2-Aminobenzoic Acid (2)

To a stirred mixture of methyl 2-aminobenzoate (1) (20 g, 132.3 mmol, 1 eq) in THF:MeOH: H₂O (2:1:1, 200 mL), LiOH.H₂O (8.32 g, 198.4 mmol, 1.5 eq) was added at RT. The reaction mixture was stirred at 50° C. for 3 h. The progress of the reaction was monitored by TLC (M.Ph: 20% EtOAc in n-hexane). The reaction mixture was concentrated under reduced pressure. The resulting residue was diluted with water (200 mL) and extracted with EtOAc (3 × 200 mL). The combined organic layers was washed with brine, dried over sodium sulfate, filtered and concentrated to dryness to afford titled compound 2 (14.6 g, 80.4%) as an off-white solid. ¹H NMR (DMSO-d6, 400 MHz): 8 8.55 (bs, 3H), 7.66 (d, J= 6.4 Hz, 1H), 7.21 (t, J= 8.0 Hz, 1H), 6.71 (d, J= 8.4 Hz, 1H), 6.49 (d, J= 6.8 Hz, 1H).

### Step-2: 2-(Isobutylamino)Benzoic Acid (4)

To a stirred mixture of 2-aminobenzoic acid (2) (5 g, 36.46 mmol, 1 eq) in 1,2 dichloro ethane (200 mL), isobutyraldehyde (3) (2.9 g, 40.1 mmol, 1.1 eq), sodium triacetoxyborohydride (30.9 g, 145.8 mmol, 4 eq) and AcOH (10.9 g, 182.3 mmol, 5 eq) were added at RT. The reaction mixture was stirred at RT for 16 h. The progress of the reaction was monitored by TLC (M.Ph: 10% EtOAc in n-hexane). The reaction mixture was concentrated under reduced pressure. The resulting residue was dissolved with DCM (150 mL) and washed with water (2 × 100 mL), followed by brine. The organic layer dried over sodium sulfate, filtered and concentrated to dryness. The crude was purified through silica gel column chromatography (elution: 0-10% EtOAc in n-hexane) to afford 4 (6.9 g, 97.94%) as oily mass. ¹H NMR (DMSO-d6, 400 MHz): 8 8.25 (bs, 2H), 7.78 (d, J= 8.0 Hz, 1H), 7.35 (t, J= 7.2 Hz, 1H), 6.72 (d, J= 8.4 Hz, 1H), 6.54 (t, J= 8.0 Hz, 1H), 3.00 (d, J= 7.2 Hz, 2H), 1.90-1.86 (m, 1H), 0.96 (d, 7.2 Hz, 6H); LC-MS: m/z 193.74 [M+H]⁺.

### Step-3: 1-Isobutyl-2H-Benzo[d][l,3]Oxazine-2,4(1H)-Dione (6)

To a stirred mixture of 2-(isobutylamino)benzoic acid (4) (6.2 g, 32.09 mmol, 1 eq) in EtOAc (200 mL), K₂CO₃ (6.85 g, 48.13 mmol, 1.5 eq) and triphosgene (4.76 g, 16.04 mmol, 0.5 eq) were added at 0° C. The reaction mixture was stirred at RT for 1 h. The progress of the reaction was monitored by TLC (M.Ph: 20% EtOAc in n-hexane). The mixture was quenched with water and layers were separated. The aqueous layer was extracted with EtOAc (100 mL). The combined organic layers was washed with brine, dried over sodium sulfate, filtered and concentrated to dryness. The crude was purified through silica gel column chromatography (elution: 10-90% EtOAc in n-hexane) to afford 6 (6.4 g, 91.15%) as an off-white solid. ¹H NMR (DMSO-d6, 400 MHz): 8 8.02 (d, J= 8.0 Hz, 1H), 7.85 (t, J= 7.2 Hz, 1H), 7.49 (d, J= 8.4 Hz, 1H), 7.34 (t, J= 7.6 Hz, 1H), 3.88 (d, J= 7.6 Hz, 2H), 2.11-2.08 (m, 1H), 0.96 (d, 6.8 Hz, 6H); LC-MS: m/z 220.12 [M+H]⁺.

### Step-4: Ethyl 4-Hydroxy-1-Isobutyl-2-Oxo-1,2-Dihydroquinoline-3-Carboxylate (8)

To a stirred solution of diethyl malonate (7) (2.84 g, 17.7 mmol, 1.3 eq) in N,N-dimethyl acetamide (50 mL), NaH (60%, 0.81 g, 20.4 mmol, 1.5 eq) was added at 0° C. The reaction mixture was allowed to RT and stirred for 15 min at same temperature. Then the mixture was heated to 110° C. and a solution of 1-isobutyl-2H-benzo[d][l,3]oxazine-2,4(1H)-dione (6) (3.0 g, 13.6 mmol, 1 eq) in N,N-dimethyl acetamide (20 mL) was added slowly at same temperature. The mixture was stirred at 110° C. for 4 h. The progress of the reaction was monitored by TLC (M.Ph: 100% DCM). The reaction mixture was quenched with chilled water and subjected to acidification upto pH 4 by carefully addition of 6 N HCI. The aqueous layer was extracted with EtOAc (2 × 100 mL). The combined organic layers was washed with brine, dried over sodium sulfate, filtered and concentrated to dryness. The crude was purified through silica gel column chromatography (elution: 100% DCM) to afford 8 (3.85 g, 97.46%) as a low melting brownish solid. ¹H NMR (DMSO-d6, 400 MHz): 8 13.02 (s, 1H), 8.07 (d, J = 8.0 Hz, 1H), 7.73 (t, J= 7.2 Hz, 1H), 7.56 (d, J= 8.8 Hz, 1H), 7.30 (t, J= 7.2 Hz, 1H), 4.35 (q, J= 7.6 Hz, 2H), 4.07 (d, J= 7.6 Hz, 2H), 2.11-2.08 (m, 1H), 1.32 (t, J= 6.8 Hz, 3H), 0.89 (d, 6.8 Hz, 6H); LC-MS: m/z 289.92 [M+H]⁺.

### Step-5: 4-Hydroxy-1-Isobutyl-N-(3-Methylpyridin-2-yl)-2-Oxo-1,2-Dihydroquinoline-3-Carboxamide (SSTN-513)

To a stirred mixture of ethyl 4-hydroxy-1-isobutyl-2-oxo-1,2-dihydroquinoline-3-carboxylate (8) (140 mg, 0.483 mmol, 1 eq) in DMSO (5 mL), 3-methylpyridin-2-amine (9) (78.4 mg, 0.725 mmol, 1.5 eq) was added at RT. The reaction mixture was stirred at 120° C. for 1 h. The progress of the reaction was monitored by TLC (M.Ph: 30% EtOAc in n-hexane). The reaction mixture was quenched with chilled water (50 mL) and extracted with EtOAc (2 × 50 mL). The combined organic layers was washed with brine, dried over sodium sulfate, filtered and concentrated to dryness. The crude was purified through silica gel column chromatography (elution: 20-80%) to afford SSTN-513 (35 mg, 20.6%) as an off-white solid. ¹H NMR (DMSO-d6, 400 MHz): 8 16.33 (s, 1H), 12.43 (s, 1H), 8.33 (d, J= 4 Hz, 1H), 8.16 (d, J= 8 Hz, 1H), 7.86 (t, J= 8 Hz, 3H), 7.43-7.39 (m, 1H), 7.30-7.27 (m, 1H), 4.23 (d, J= 6.4 Hz, 2H), 2.38 (s, 3H), 2.19-2.16 (m, 1H), 0.98 (d, J= 6.4 Hz, 6H); LC-MS: m/z 352.0 [M+H]⁺; HPLC: 99.72%.

SSTN-514

Synthesis of 4-hydroxy-1-isobutyl-6-methoxy-N-(3-methylpyridin-2-yl)-2-oxo-1,2-dihydroquinoline-3-carboxamide (SSTN-514)

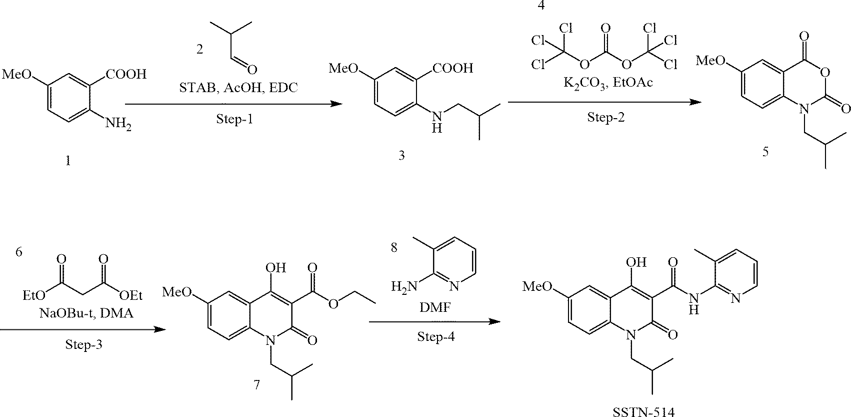

### Step-1: 2-(Isobutylamino)-5-Methoxybenzoic Acid (3)

To a stirred mixture of 2-amino-5-methoxybenzoic acid (1) (4 g, 23.92 mmol, 1 eq) in 1,2 dichloro ethane (200 mL), isobutyraldehyde (2) (2.85 g, 31.1 mmol, 1.3 eq), sodium triacetoxyborohydride (20.2 g, 95.7 mmol, 4 eq) and AcOH (4.1 mL, 71.7 mmol, 3 eq) were added at RT. The reaction mixture was stirred for 16 h at RT. The progress of the reaction was monitored by TLC (M.Ph: 10% EtOAc in n-hexane). The reaction mixture was concentrated under reduced pressure. The resulting residue was dissolved with DCM (150 mL) and washed with water (2 × 100 mL), followed by brine. The organic layer dried over sodium sulfate, filtered and concentrated to dryness. The crude was purified through silica gel column chromatography (elution: 0-30% EtOAc in n-hexane) to afford 3 (3.19 g, 59.7%) as oily mass.

### Step-2: 1-Isobutyl-6-Methoxy-2H-Benzo[d][1,3]Oxazine-2,4(1H)-Dione (5)

To a stirred mixture of 2-(isobutylamino)-5-methoxybenzoic acid (3) (3 g, 13.4 mmol, 1 eq) in EtOAc (150 mL), K₂CO₃ (2.77 g, 20.1 mmol, 1.5 eq) and triphosgene (4) (1.99 g, 6.7 mmol, 0.5 eq) were added at 0° C. The reaction mixture was stirred at RT for 1 h. The progress of reaction was monitored by TLC (M.Ph: 20% EtOAc in n-hexane). The mixture was quenched with water and layers were separated. The aqueous layer was extracted with EtOAc (100 mL). The combined organic layers was washed with brine, dried over sodium sulfate, filtered and concentrated to dryness. The crude 5 (3.05 g, 91.31%) was taken to next step without purification. ¹H NMR (DMSO-A, 400 MHz): 8 7.46-7.40 (m, 3H), 3.85-3.80 (m, 5H), 2.11-2.04 (m, 1H), 0.94 (d, 6.8 Hz, 6H); LC-MS: m/z 250.15 [M+H]⁺.

### Step-3: Ethyl 4-Hydroxy-1-Isobutyl-6-Methoxy-2-Oxo-1,2-Dihydroquinoline-3-Carboxylate (7)

To a stirred solution of diethyl malonate (6) (2.44 mL, 16.0 mmol, 2 eqv) in N,N-dimethyl acetamide (10 mL), t-BuONa (1.54 g, 16.0 mmol, 2 eqv) was added at 0° C. After 10 min of stirring 1-isobutyl-6-methoxy-2H-benzo[d][1,3]oxazine-2,4(1H)-dione (5) was added. The mixture was stirred at 90° C. for 16 h. The progress of the reaction was monitored by TLC (M.Ph: 50% EtOAc in n-hexane). The reaction mixture was quenched with chilled water (150 mL) and extracted with EtOAc (2 × 100 mL). The combined organic layers was washed with brine, dried over sodium sulfate, filtered and concentrated to dryness. The crude was purified through silica gel column chromatography (elution: 10-50% EtOAc in n-hexane) to afford 7 (1.63, 65.2%) as an off-white solid. ¹H NMR (DMSO-d6, 400 MHz): 8 13.06 (s, 1H), 7.52-7.47 (m, 2H), 7.35-7.30 (m, 1H), 4.37 (q, J= 7.6 Hz, 2H), 4.07 (d, J= 7.6 Hz, 2H), 3.47 (s, 3H), 2.16-2.08 (m, 1H), 1.31 (t, J= 6.8 Hz, 3H), 0.89 (d, 6.8 Hz, 6H); LC-MS: m/z 319.79 [M+H]⁺.

### Step-4: 4-Hydroxy-1-Isobutyl-6-Methoxy-N-(3-Methylpyridin-2-yl)-2-Oxo-1,2-Dihydroquinoline-3-Carboxamide (SSTN-514)

To a stirred mixture of ethyl 4-hydroxy-1-isobutyl-6-methoxy-2-oxo-1,2-dihydroquinoline-3-carboxylate (7) (1 g, 3.43 mmol, 1 eq) in DMF (15 mL), 3-methylpyridin-2-amine (8) (0.556 mg, 5.4 mmol, 1.5 eq) was added at RT. The reaction mixture was stirred at 130° C. for 2 h. The progress of the reaction was monitored by TLC (M.Ph: 20% EtOAc in n-hexane). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 0-20% EtOAc in n-hexane) to afford SSTN-514 (45 mg, 3.4%) as an off-white solid. ¹H NMR (DMSO-d6, 400 MHz): 8 16.64 (s, 1H), 12.60 (s, 1H), 8.31 (d, J= 4 Hz, 1H), 7.76 (d, J= 7.2 Hz, 1H), 7.68 (d, J= 9.2 Hz, 1H), 7.51 (s, 1H), 7.45 (d, J= 9.2 Hz, 1H), 7.27-7.24 (m, 1H), 4.19 (d, J= 6.0 Hz, 2H), 3.85 (s, 3H), 2.27 (s, 3H), 2.15-2.11 (m, 1H), 0.92 (d, J= 6.4 Hz, 6H); LC-MS: m/z 382.20 [M+H]⁺; HPLC: 99.55%.

SSTN-517

Synthesis of 4-hydroxy-1-isobutyl-N-(3-methylisoxazol-5-yl)-2-oxo-1,2-dihydroquinoline-3-carboxamide (SSTN-517)

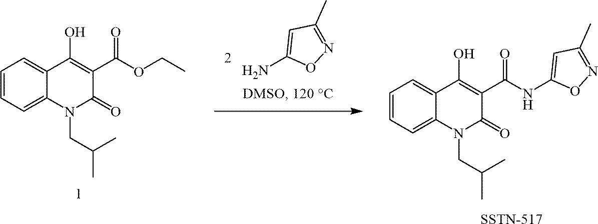

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 3-methylisoxazol-5-amine (2) (74 mg, 0.76 mmol, 1.1 eq) was added at RT. The reaction mixture was stirred at 120° C. for 1 h. The progress of the reaction was monitored by TLC (M.Ph: 20% EtOAc in n-hexane). The reaction mixture was quenched with chilled water (50 mL) and extracted with EtOAc (2 × 50 mL). The combined organic layers was washed with brine, dried over sodium sulfate, filtered and concentrated to dryness. The crude was purified through silica gel column chromatography (elution: 100% DCM) to afford SSTN-517 (42 mg, 17.87%) as an off-white solid. ¹H NMR (DMSO-d6, 400 MHz): 8 15.41 (s, 1H), 13.64 (s, 1H), 8.19 (d, J= 8.4 Hz, 1H), 7.87 (d, J= 8.0 Hz, 1H), 7.77 (d, J= 8.8 Hz, 1H), 7.46 (t, J= 7.2 Hz, 1H), 6.34 (s, 1H), 4.22 (d, J= 6.4 Hz, 2H), 2.24 (s, 3H), 2.17-2.16 (m, 1H), 0.94 (d, J= 6.8 Hz, 6H); LC-MS: m/z 341.9 [M+H]⁺; HPLC: 98.99%.

SSTN-518

Synthesis of 4-hydroxy-1-isobutyl-2-oxo-N-(pyridin-2-yl)-1,2-dihydroquinoline-3-carboxamide (SSTN-518)

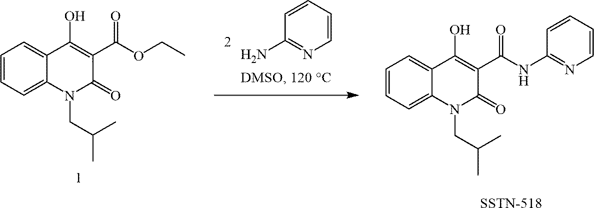

To a stirred mixture of ethyl 4-hydroxy-1-isobutyl-2-oxo-1,2-dihydroquinoline-3-carboxylate (1) (300 mg, 1.03 mmol, 1 eq) in DMSO (5 mL), 2-amino pyridine (2) (146 mg, 1.55 mmol, 1.5 eq) was added at RT. The reaction mixture was stirred at 120° C. for 1 h. The progress of the reaction was monitored by TLC (M.Ph: 20% EtOAc in n-hexane). The reaction mixture was quenched with chilled water (50 mL) and extracted with EtOAc (2 × 50 mL). The combined organic layers was washed with brine, dried over sodium sulfate, filtered and concentrated to dryness. The crude was purified through silica gel column chromatography (elution: 100% DCM) to afford SSTN-518 (180 mg, 51.5%) as an off-white solid. ¹H NMR (DMSO-d6, 400 MHz): 8 16.27 (s, 1H), 13.05 (s, 1H), 8.41 (d, J= 4 Hz, 1H), 8.18 (d, J= 8.4 Hz, 2H), 7.92-7.82 (m, 2H), 7.44 (t, J= 7.2 Hz, 1H), 7.25-7.22 (m, 1H), 4.23 (d, J= 6.8 Hz, 2H), 2.18-2.14 (m, 1H), 0.95 (d, J= 6.8 Hz, 6H); LC-MS: m/z 338.0 [M+H]⁺; HPLC: 99.17%.

SSTN-519

Synthesis of 4-hydroxy-N-(3-hydroxypyridin-2-yl)-1-isobutyl-2-oxo-1,2-dihydroquinoline-3-carboxamide (SSTN-519)

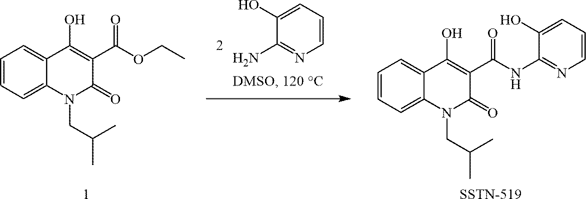

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 2-amino-3-hydroxypyridine (2) (114 mg, 1.03 mmol, 1.5 eq) was added at RT. The reaction mixture was stirred at 120° C. for 1 h. The progress of the reaction was monitored by TLC (M.Ph: 20% EtOAc in n-hexane). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 100% DCM) to afford SSTN-519 (123 mg, 50.44%) as an off-white solid. ¹H NMR (DMSO-d6, 400 MHz): 8 16.89 (s, 1H), 12.66 (s, 1H), 10.39 (s, 1H), 8.17 (d, J= 7.6 Hz, 1H), 7.92 (d, J= 4 Hz, 1H), 7.84 (t, J= 7.6 Hz, 1H), 7.72 (d, J= 8.4 Hz, 1H), 7.42 (t, J= 7.6 Hz, 1H), 7.32 (d, J= 8 Hz, 1H), 7.16-7.12 (m, 1H), 4.22 (d, J= 6.4 Hz, 2H), 2.19-2.14 (m, 1H), 0.94 (d, J= 6.8 Hz, 6H); LC-MS: m/z 354.0 [M+H]⁺; HPLC: 96.27%.

SSTN-522

Synthesis of N-(4-fluoropyridin-2-yl)-4-hydroxy-1 -isobutyl-2-oxo-1,2-dihydroquinoline-3-carboxamide (SSTN-522)

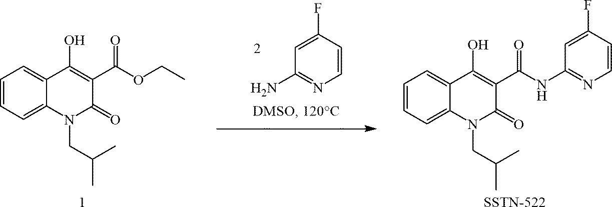

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 2-amino-4-fluoropyridine (2) (116 mg, 1.03 mmol, 1.5 eq) was added at RT. The reaction mixture was stirred at 120° C. for 1 h. The progress of the reaction was monitored by TLC (M.Ph: 20% EtOAc in n-hexane). The reaction mixture was quenched with chilled water (50 mL) and extracted with EtOAc (2 × 50 mL). The combined organic layers was washed with brine, dried over sodium sulfate, filtered and concentrated to dryness. The crude was purified through silica gel column chromatography (elution: 100% DCM) to afford SSTN-522 (95 mg, 38.77%) as an off-white solid. ¹H NMR (DMSO-d6, 400 MHz): 8 15.92 (s, 1H), 13.26 (s, 1H), 8.45 (dd, J= 6 Hz, J= 3.2 Hz, 1H) 8.18 (d, J= 8.4 Hz, 1H), 7.99-7.96 (m, 1H), 7.86-7.83 (m, 1H), 7.73 (d, J= 8.8 Hz, 1H), 7.44 (t, J= 7.2 Hz, 1H), 7.19-7.18 (m, 1H), 4.22 (d, J= 6.8 Hz, 2H), 2.17-2.14 (m, 1H), 0.95 (d, J= 6.8 Hz, 6H); LC-MS: m/z 356.0 [M+H]⁺; HPLC: 99.92%.

SSTN-525

Synthesis of 4-hydroxy-1-isobutyl-N-(isoxazol-3-yl)-2-oxo-1,2-dihydroquinoline-3-carboxamide (SSTN-525)

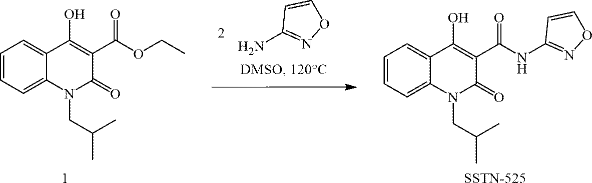

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), isoxazol-3-amine (2) (87.2 mg, 1.03 mmol, 1.5 eq) was added at RT. The reaction mixture was stirred at 120° C. for 1 h. The progress of the reaction was monitored by TLC (M.Ph: 20% EtOAc in n-hexane). The reaction mixture was quenched with chilled water (100 mL) and extracted with EtOAc (2 × 70 mL). The combined organic layers was washed with brine, dried over sodium sulfate, filtered and concentrated to dryness. The crude was purified through silica gel column chromatography (elution: 100% DCM) to afford SSTN-525 (62 mg, 27.4%) as an off-white solid. ¹H NMR (DMSO-d6, 400 MHz): 8 15.86 (s, 1H), 13.22 (s, 1H), 8.95 (s, 1H), 8.18 (d, J = 8.4 Hz, 1H), 7.88 (t, J= 7.6 Hz, 1H), 7.75 (d, J= 8.8 Hz, 1H), 7.45 (t, J= 7.6 Hz, 1H), 7.07 (s, 1H), 4.23 (d, J= 6.8 Hz, 2H), 2.17-2.14 (m, 1H), 0.95 (d, J= 7.2 Hz, 6H); LC-MS: m/z 327.9 [M+H]⁺; HPLC: 99.83%.

SSTN-526

Synthesis of N-(4-chloropyridin-2-yl)-4-hydroxy-1 -isobutyl-2-oxo-1,2-dihydroquinoline-3-carboxamide (SSTN-526)

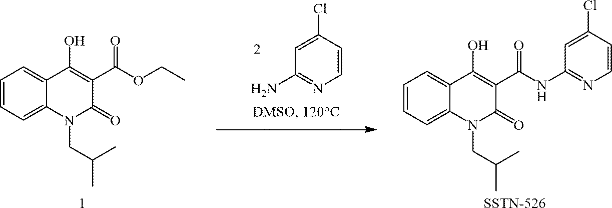

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 4-chloropyridin-2-amine (2) (133 mg, 1.03 mmol, 1.5 eqv) was added at RT. The reaction mixture was stirred at 120° C. for 1 h. The reaction was monitored by TLC (M.Ph: 20% EtOAc in n-hexane). The reaction mixture was quenched with chilled water (50 mL) and extracted with EtOAc (2 × 100 mL). The combined organic layers was washed with brine, dried over sodium sulfate, filtered and concentrated to dryness. The crude was purified through silica gel column chromatography (elution: 100% DCM) to afford SSTN-526 (45 mg, 17.5%) as an off-white solid. ¹H NMR (DMSO-d6, 400 MHz): 8 15.88 (s, 1H), 13.22 (s, 1H), 8.40 (d, J= 5.2 Hz, 1H), 8.23 (s, 1H), 8.18 (d, J= 8.4 Hz, 1H), 7.87 (t, J= 7.6 Hz, 1H), 7.73 (d, J= 8.8 Hz, 1H), 7.44-7.37 (m, 2H), 4.22 (d, J= 7.2 Hz, 2H), 2.18-2.14 (m, 1H), 0.95 (d, J= 6.4 Hz, 6H); LC-MS: m/z 371.9 [M+H]⁺; HPLC: 98.24%.

SSTN-527

Synthesis of 4-hydroxy-1 -isobutyl-N-(4-morpholinopyridin-2-yl)-2-oxo-1,2-dihydroquinoline-3-carboxamide (SSTN-527)

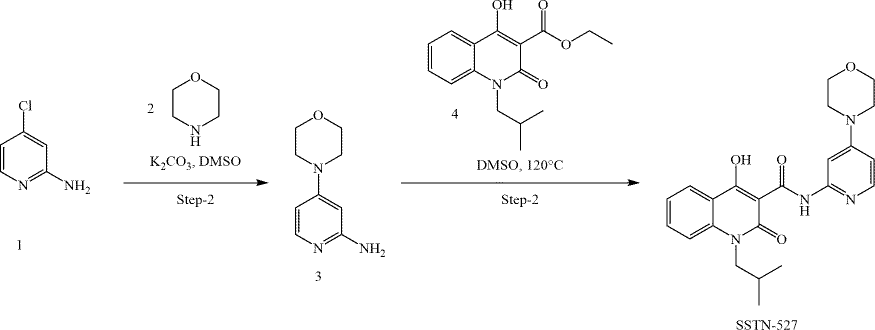

### Step-1: 4-Morpholinopyridin-2-Amine (3)

To a stirred mixture of 2-amino-4-chloropyridine (1) (200 mg, 1.55 mmol, 1 eq) in DMSO (5 mL), morpholine (2) (203 mg, 2.33 mmol, 1.5 eq) and K₂C03 (427 mg, 3.1 mmol, 2 eq) were added at RT. The reaction mixture was stirred at 140° C. for 6 h. The progress of the reaction was monitored by TLC (M.Ph: 10% MeOH in DCM). The reaction mixture was quenched with chilled water (100 mL) and extracted with EtOAc (2 × 70 mL). The combined organic layers was washed with brine, dried over sodium sulfate, filtered and concentrated to dryness. The crude was purified through silica gel column chromatography (elution: 100% DCM) to afford 3 (150 mg, 53.9%) as an off-white solid. ¹H NMR (DMSO-d6, 400 MHz): 8 7.86 (d, J= 5.2 Hz, 1H), 7.61 (d, J= 7.2 Hz, 1H), 6.22 (s, 1H), 5.49 (bs, 2H), 3.69 (t, J= 4.8 Hz, 4H), 3.12 (t, J= 4.8 Hz, 4H); LC-MS: m/z 179.8 [M+H]⁺.

### Step-2: 4-Hydroxy-1-Isobutyl-N-(4-Morpholinopyridin-2-yl)-2-Oxo-1,2-Dihydroquinoline-3-Carboxamide (SSTN-527)

To a stirred mixture of ethyl 4-hydroxy-1-isobutyl-2-oxo-1,2-dihydroquinoline-3-carboxylate (1) (121 mg, 0.418 mmol, 1.0 eq) in DMSO (2 mL), 4-morpholinopyridin-2-amine (3) (100 mg, 0.558 mmol, 1.3 eq) was added at RT. The reaction mixture was stirred at 120° C. for 1 h. The reaction was monitored by TLC (M.Ph: 5% MeOH in DCM). The reaction mixture was quenched with chilled water (50 mL) and extracted with EtOAc (2 × 50 mL). The combined organic layers were washed with brine, dried over sodium sulfate, filtered and concentrated to dryness. The crude was purified through silica gel column chromatography (elution: 0-2% MeOH in DCM) to afford SSTN-527 (5 mg, 2.12%) as an off-white solid. ¹H NMR (DMSO-d6, 400 MHz): 8 16.36 (s, 1H), 13.00 (s, 1H), 8.28 (d, J= 7.6 Hz, 1H), 8.13 (d, J= 6.0 Hz, 1H), 7.76 (s, 1H), 7.71 (t, J= 9.6 Hz, 1H), 7.38 (t, J= 8.4 Hz, 1H), 7.30-7.27 (m, 1H), 6.50 (d, J= 3.6 Hz, 1H), 4.22 (d, J= 6.8 Hz, 2H), 3.89 (t, J= 4.4 Hz, 4H), 3.39 (t, J= 4.4 Hz, 4H), 2.29-2.22 (m, 1H), 1.02 (d, J= 6.8 Hz, 6H); LC-MS: m/z 423.20 [M+H]⁺; HPLC: 99.50%.

SSTN-528

Synthesis of 4-hydroxy-1-isobutyl-N-(oxazol-2-yl)-2-oxo-1,2-dihydroquinoline-3-carboxamide (SSTN-528)

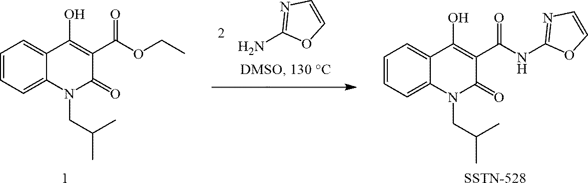

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), oxazol-2-amine (2) (69.7 mg, 0.829 mmol, 1.2 eq) was added at RT. The reaction mixture was stirred at 130° C. for 2 h. The reaction was monitored by TLC (M.Ph: 20% EtOAc in n-hexane). The reaction mixture was quenched with chilled water (70 mL) and extracted with EtOAc (2 x 50 mL). The combined organic layers was washed with brine, dried over sodium sulfate, filtered and concentrated to dryness. The crude was purified by Prep HPLC to afford SSTN-528 (22 mg, 9.73%) as an off-white solid. ¹H NMR (DMSO-d6, 400 MHz): 8 15.88 (s, 1H), 13.55 (s, 1H), 8.18 (d, J= 8.0 Hz, 1H), 8.01 (s, 1H), 7.89 (t, J= 8.0 Hz, 1H), 7.76 (d, J= 8.4 Hz, 1H), 7.46 (t, J= 7.6 Hz, 1H), 7.23 (s, 1H), 4.22 (d, J= 6.4 Hz, 2H), 2.17-2.14 (m, 1H), 0.94 (d, J= 6.0 Hz, 6H); LC-MS: m/z 328.20 [M+H]⁺; HPLC: 98.43%.

SSTN-532

Synthesis of 4-hydroxy-1-isobutyl-N-(4-methoxypyridin-2-yl)-2-oxo-1,2-dihydroquinoline-3-carboxamide (SSTN-532)

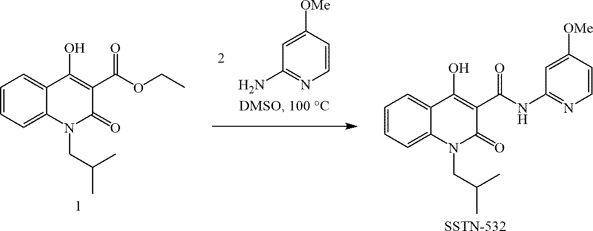

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 4-methoxypyridin-2-amine (2) (128.6 mg, 1.03 mmol, 1.5 eq) was added at RT. The reaction mixture was stirred at 100° C. for 2 h. The reaction was monitored by TLC (M.Ph: 20% EtOAc in n-hexane). The reaction mixture was quenched with chilled water (50 mL) and extracted with EtOAc (2 x 70 mL). The combined organic layers was washed with brine, dried over sodium sulfate, filtered and concentrated to dryness. The crude was purified by Prep HPLC to afford SSTN-532 (46.4 mg, 18.27%) as an off-white solid. ¹H NMR (DMSO-A, 400 MHz): 8 16.19 (s, 1H), 13.01 (s, 1H), 8.22 (d, J= 6.0 Hz, 1H), 8.17 (d, J= 6.8 Hz, 1H), 7.84 (t, J= 8.0 Hz, 1H), 7.75-7.70 (m, 3H), 7.43 (t, J= 7.2 Hz, 1H), 6.85 (dd, J= 3.6 Hz, J= 2.4 Hz, 1H), 4.22 (d, J= 6.4 Hz, 2H), 2.17-2.14 (m, 1H), 0.94 (d, J= 6.0 Hz, 6H); LC-MS: m/z 368.0 [M+H]⁺; HPLC: 99.82%.

SSTN-533

Synthesis of 4-hydroxy-1-isobutyl-N-(1-methyl-1H-pyrazol-3-yl)-2-oxo-1,2-dihydroquinoline-3-carboxamide (SSTN-533)

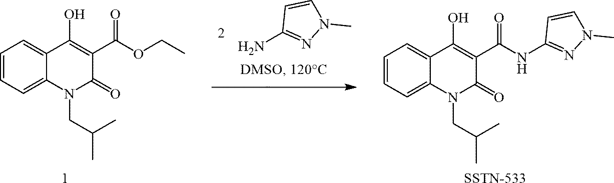

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 1-methyl-1H-pyrazol-3-amine (2) (100 mg, 1.03 mmol, 1.5 eq) was added at RT. The reaction mixture was stirred at 120° C. for 2 h. The progress of the reaction was monitored by TLC (M.Ph: 20% EtOAc in n-hexane). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 0-2% MeOH in DCM) to afford SSTN-533 (62 mg, 26.38%) as an off-white solid. ¹H NMR (DMSO-A, 400 MHz): 8 16.59 (s, 1H), 12.80 (s, 1H), 8.15 (d, J= 8.0 Hz, 1H), 7.83 (t, J= 7.2 Hz, 1H), 7.72-7.68 (m, 2H), 7.42 (t, J= 7.6 Hz, 1H), 6.57 (d, J= 2.0 Hz, 1H), 4.21 (d, J= 6.8 Hz, 2H), 3.79 (s, 3H), 2.17-2.14 (m, 1H), 0.94 (d, J= 6.8 Hz, 6H); LC-MS: m/z 341.3 [M+H]⁺; HPLC: 98.48%.

SSTN-534

Synthesis of 4-hydroxy-1-isobutyl-N-(1-methyl-1H-pyrazol-4-yl)-2-oxo-1,2-dihydroquinoline-3-carboxamide (SSTN-534)

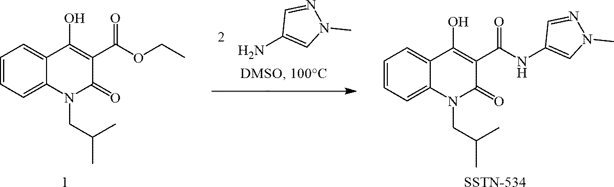

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 1-methyl-1H-pyrazol-4-amine (2) (100 mg, 1.03 mmol, 1.5 eq) was added at RT. The reaction mixture was stirred at 100° C. for 2 h. The reaction was monitored by TLC (M.Ph: 5% MeOH in DCM). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 0-2% MeOH in DCM) to afford SSTN-534 (83.22 mg, 35.39%) as an off-white solid. ¹H NMR (DMSO-A, 400 MHz): 8 16.80 (s, 1H), 12.32 (s, 1H), 8.15-8.12 (m, 2H), 7.83 (t, J= 7.8 Hz, 1H), 7.72-7.70 (m, 2H), 7.41 (t, J= 7.2 Hz, 1H), 4.20 (d, J= 6.8 Hz, 2H), 3.84 (s, 3H), 2.20-2.13 (m, 1H), 0.93 (d, J= 6.4 Hz, 6H); LC-MS: m/z 341.35 [M+H]⁺; HPLC: 99.55%.

SSTN-535

Synthesis of 4-hydroxy-1-isobutyl-2-oxo-N-(o-tolyl)-1,2-dihydroquinoline-3-carboxamide (SSTN-535)

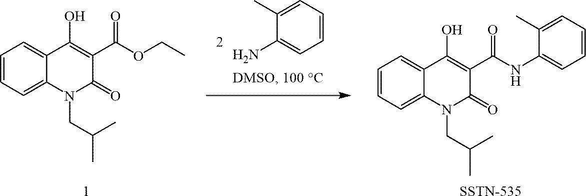

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), o-toluidine (2) (88.7 mg, 0.829 mmol, 1.2 eq) was added at RT. The reaction mixture was stirred at 100° C. for 2 h. The reaction was monitored by TLC (M.Ph: 20% EtOAc in n-hexane). The reaction mixture was quenched with chilled water (50 mL) and extracted with EtOAc (2 × 100 mL). The combined organic layers was washed with brine, dried over sodium sulfate, filtered and concentrated to dryness. The crude was purified through silica gel column chromatography (elution: 100% DCM) to afford SSSTN-535 (125 mg, 51.65%) as an off-white solid. ¹H NMR (DMSO-d6, 400 MHz): 8 16.78 (s, 1H), 12.57 (s, 1H), 8.17 (d, J= 7.6 Hz, 1H), 8.07 (d, J= 8.4 Hz, 1H), 7.85 (t, J= 7.2 Hz, 1H), 7.74 (d, J= 8.4 Hz, 1H), 7.43 (t, J= 8.0 Hz, 1H), 7.32-7.24 (m, 2H), 7.15 (t, J= 7.6 Hz, 1H), 4.23 (d, J= 7.2 Hz, 2H), 2.36 (s, 3H), 2.19-2.14 (m, 1H), 0.94 (d, J= 6.4 Hz, 6H); LC-MS: m/z 351.22 [M+H]⁺; HPLC: 99.83%.

SSTN-537

Synthesis ofN-(2-fluorophenyl)-4-hydroxy-1-isobutyl-2-oxo-1,2-dihydroquinoline-3-carboxamide (SSTN-537)

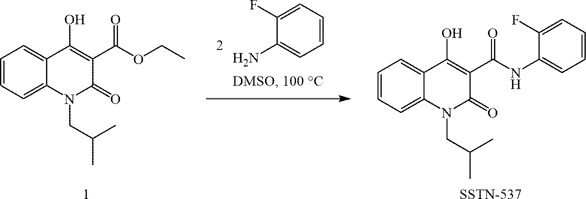

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 2-fluoroaniline (2) (115 mg, 1.03 mmol, 1.5 eq) was added at RT. The reaction mixture was stirred at 100° C. for 2 h. The reaction was monitored by TLC (M.Ph: 100% DCM). The reaction mixture was quenched with chilled water (100 mL) and extracted with EtOAc (2 x 100 mL). The combined organic layers was washed with brine, dried over sodium sulfate, filtered and concentrated to dryness. The crude was purified through silica gel column chromatography (elution: 100% DCM) to afford SSTN-537 (65 mg, 26.5%) as an off-white solid. ¹H NMR (DMSO-d6, 400 MHz): 8 16.32 (s, 1H), 12.95 (s, 1H), 8.30 (t, J= 6.8 Hz, 1H), 8.15 (d, J= 8.4 Hz, 1H), 7.85 (t, J= 8.0 Hz, 1H), 7.75 (d, J= 8.8 Hz, 1H), 7.44-7.35 (m, 2H), 7.27-7.22 (m, 2H), 4.23 (d, J= 6.0 Hz, 2H), 2.20-2.12 (m, 1H), 0.93 (d, J= 6.8 Hz, 6H); LC-MS: m/z 355.0 [M+H]⁺; HPLC: 99.83%.

SSTN-538

Synthesis of N-(3 -fluorophenyl)-4-hydroxy-1 -isobutyl-2-oxo-1,2-dihydroquinoline-3-carboxamide (SSTN-538)

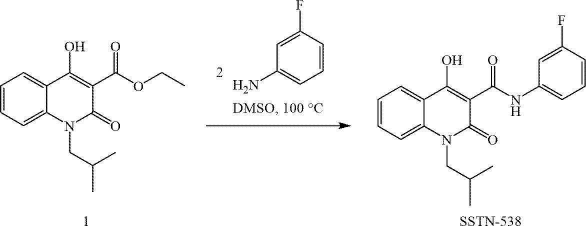

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 3-fluoroaniline (2) (115 mg, 1.03 mmol, 1.5 eq) was added at RT. The reaction mixture was stirred at 100° C. for 2 h. The reaction was monitored by TLC (M.Ph: 30% EtOAc in n-hexane). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 0-20% EtOAc in n-hexane) to afford SSTN-538 (78 mg, 31.96%) as an off-white solid. ¹H NMR (DMSO-d6, 400 MHz): 8 16.34 (s, 1H), 12.82 (s, 1H), 8.17 (d, J= 8.0 Hz, 1H), 7.86 (t, J= 7.6 Hz, 1H), 7.74 (t, J= 8.8 Hz, 2H), 7.47-7.39 (m, 3H), 7.05 (d, J= 7.6 Hz, 1H), 4.22 (d, J= 6.8 Hz, 2H), 2.21-2.14 (m, 1H), 0.94 (d, J= 6.4 Hz, 6H); LC-MS: m/z 355.10 [M+H]⁺; HPLC: 99.01%.

SSTN-539

Synthesis ofN-(4-fluorophenyl)-4-hydroxy-1-isobutyl-2-oxo-1,2-dihydroquinoline-3-carboxamide (SSTN-539)

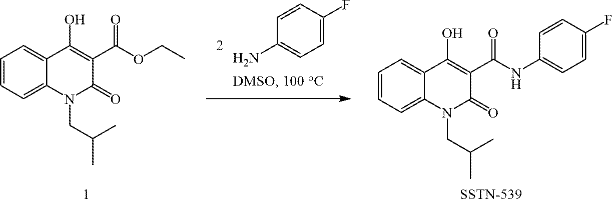

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 4-fluoroaniline (2) (115 mg, 1.03 mmol, 1.5 eq) was added at RT. The reaction mixture was stirred at 100° C. for 2 h. The reaction was monitored by TLC (M.Ph: 30% EtOAc in n-hexane). The reaction mixture was quenched with chilled water (100 mL) and extracted with EtOAc (2 × 100 mL). The combined organic layers was washed with brine, dried over sodium sulfate, filtered and concentrated to dryness. The crude was purified by Prep HPLC to afford SSTN-539 (83.94 mg, 33.97%) as an off-white solid. ¹H NMR (DMSO-A, 400 MHz): 8 16.57 (s, 1H), 12.67 (s, 1H), 8.17 (d, J= 7.6 Hz, 1H), 7.85 (t, J= 8.4 Hz, 1H), 7.73-7.70 (m, 3H), 7.43 (t, J= 8.0 Hz, 1H), 7.27 (t, J= 8.8 Hz, 2H), 4.23 (d, J= 6.4 Hz, 2H), 2.19-2.16 (m, 1H), 0.94 (d, J= 6.8 Hz, 6H); LC-MS: m/z 354.9 [M+H]⁺; HPLC: 98.28%.

SSTN-540

Synthesis of 4-hydroxy-1-isobutyl-N-(2-methylpyridin-3-yl)-2-oxo-1,2-dihydroquinoline-3-carboxamide (SSTN-540)

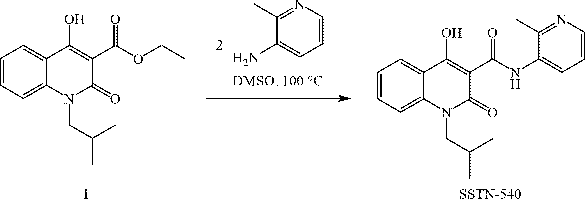

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 2-methylpyridin-3-amine (2) (112.1 mg, 1.03 mmol, 1.5 eq) was added at RT. The reaction mixture was stirred at 100° C. for 2 h. The reaction was monitored by TLC (M.Ph: 20% EtOAc in n-hexane). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 0-20% EtOAc in n-hexane) to afford SSTN-540 (71 mg, 29.33%) as an off-white solid. ¹H NMR (DMSO-d6, 400 MHz): 8 16.42 (s, 1H), 12.69 (s, 1H), 8.39 (d, J= 7.6 Hz, 1H), 8.29 (d, J= 4.4 Hz, 1H), 8.17 (d, J= 8.4 Hz, 1H), 7.86 (t, J= 7.6 Hz, 1H), 7.74 (d, J= 8.8 Hz, 1H), 7.43 (t, J= 7.2 Hz, 1H), 7.31-7.28 (m, 1H), 4.24 (d, J= 6.8 Hz, 2H), 2.58 (s, 3H), 2.19-2.16 (m, 1H), 0.94 (d, J= 7.2 Hz, 6H); LC-MS: m/z 351.9 [M+H]⁺; HPLC: 99.41 %.

SSTN-541

Synthesis ofN-(furan-2-ylmethyl)-4-hydroxy-1-isobutyl-2-oxo-1,2-dihydroquinoline-3-carboxamide (SSTN-541)

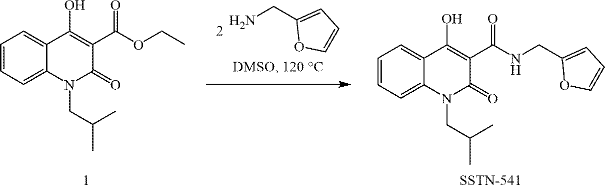

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), furfuryl amine (2) (100 mg, 1.03 mmol, 1.5 eq) was added at RT. The reaction mixture was stirred at 120° C. for 2 h. The reaction was monitored by TLC (M.Ph: 100% DCM). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 100% DCM) to afford SSTN-541 (177 mg, 75.3%) as an off-white solid. ¹H NMR (DMSO-A, 400 MHz): 8 17.10 (s, 1H), 10.66 (t, J= 5.6 Hz, 1H), 8.11 (d, J= 8.4 Hz, 1H), 7.80 (t, J= 8.8 Hz, 1H), 7.66-7.62 (m, 2H), 7.37 (t, J= 7.2 Hz, 1H), 6.42-6.36 (m, 2H), 4.60 (d, J= 5.6 Hz, 2H), 4.13 (d, J= 7.2 Hz, 2H), 2.12-2.09 (m, 1H), 0.88 (d, J= 6.8 Hz, 6H); LC-MS: m/z 341.10 [M+H]⁺; HPLC: 99.84%.

SSTN-549

Synthesis of 4-hydroxy-1-isobutyl-N-(5-methyl-1H-pyrazol-3-yl)-2-oxo-1,2-dihydroquinoline-3-carboxamide (SSTN-549)

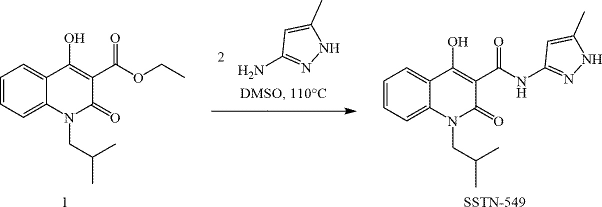

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 5-methyl-1H-pyrazol-3-amine (2) (100 mg, 1.03 mmol, 1.5 eq) was added at RT. The reaction mixture was stirred at 110° C. for 24 h. The reaction was monitored by TLC (M.Ph: 5% MeOH in DCM). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 0-2% MeOH in DCM) to afford SSTN-549 (90 mg, 38.2%) as an off-white solid. ¹H NMR (DMSO-d6, 400 MHz): 8 16.76 (s, 1H), 12.68 (s, 1H), 12.34 (s, 1H), 8.14 (s, 1H), 7.82 -7.71 (m, 2H), 7.40 (s, 1H), 6.41 (s, 1H), 4.20 (d, J= 6.8 Hz, 2H), 2.24 (s, 3H), 2.17-2.14 (m, 1H), 0.93 (d, J= 6.8 Hz, 6H); LC-MS: m/z 341.3 [M+H]⁺; HPLC: 98.82%.

SSTN-550

Synthesis of 4-hydroxy-1-isobutyl-N-(4-methylpyridin-3-yl)-2-oxo-1,2-dihydroquinoline-3-carboxamide (SSTN-550)

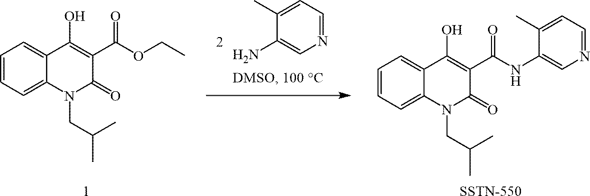

To a stirred mixture of ethyl 4-hydroxy-1-isobutyl-2-oxo-1,2-dihydroquinoline-3-carboxylate (1) (250 mg, 0.864 mmol, 1 eq) in DMSO (5 mL), 4-methylpyridin-3-amine (2) (140 mg, 1.29 mmol, 1.5 eq) was added at RT. The reaction mixture was stirred at 100° C. for 5 h. The reaction was monitored by TLC (M.Ph: 30% EtOAc in n-hexane). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 0-20% EtOAc in n-hexane) to afford SSTN-550 (128 mg, 42.24%) as an off-white solid. ¹H NMR (DMSO-d6, 400 MHz): 8 16.45 (s, 1H), 12.59 (s, 1H), 9.11 (s, 1H), 8.31 (d, J = 4.4 Hz, 1H), 8.18 (d, J= 8.0 Hz, 1H), 7.87 (t, J= 7.2 Hz, 1H), 7.76 (d, J= 8.8 Hz, 1H), 7.44 (t, J= 6.8 Hz, 1H), 7.37 (d, J= 4.4 Hz, 1H), 4.24 (d, J= 5.2 Hz, 2H), 2.36 (s, 3H), 2.20-2.16 (m, 1H), 0.94 (d, J= 6.4 Hz, 6H); LC-MS: m/z 352.15 [M+H]⁺; HPLC: 99.60%.

SSTN-551

Synthesis of 4-hydroxy-1-isobutyl-N-(3-methylpyridin-4-yl)-2-oxo-1,2-dihydroquinoline-3-carboxamide (SSTN-551)

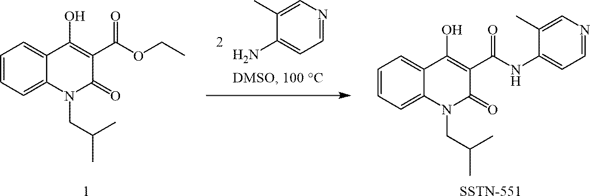

To a stirred mixture of ethyl 4-hydroxy-1-isobutyl-2-oxo-1,2-dihydroquinoline-3-carboxylate (1) (200 mg, 0.691 mmol, 1 eq) in DMSO (5 mL), 3-methylpyridin-4-amine (2) (112.1 mg, 1.03 mmol, 1.5 eq) was added at RT. The reaction mixture was stirred at 100° C. for 3 h. The reaction was monitored by TLC (M.Ph: 30% EtOAc in n-hexane). The reaction mixture was quenched with chilled water (100 mL) and extracted with EtOAc (2 × 100 mL). The combined organic layers was washed with brine, dried over sodium sulfate, filtered and concentrated to dryness. The crude was purified by Prep HPLC to afford SSTN-551 (26.15 mg, 10.7%) as an off-white solid. ¹H NMR (DMSO-A, 400 MHz): 8 16.14 (s, 1H), 12.97 (s, 1H), 8.46 (s, 1H), 8.42 (d, J= 5.2 Hz, 1H), 8.21-8.13 (m, 2H), 7.87 (t, J= 7.2 Hz, 1H), 7.76 (d, J= 8.8 Hz, 1H), 7.45 (t, J= 7.2 Hz, 1H), 4.24 (d, J= 6.8 Hz, 2H), 2.36 (s, 3H), 2.19-2.16 (m, 1H), 0.95 (d, J= 7.2 Hz, 6H); LC-MS: m/z 351.9 [M+H]⁺; HPLC: 98.29%.

SSTN-552

Synthesis of 4-hydroxy-1-isobutyl-2-oxo-N-(3-(piperazin-1-yl)phenyl)-1,2-dihydroquinoline-3-carboxamide trifluoro aceticacid (SSTN-552)

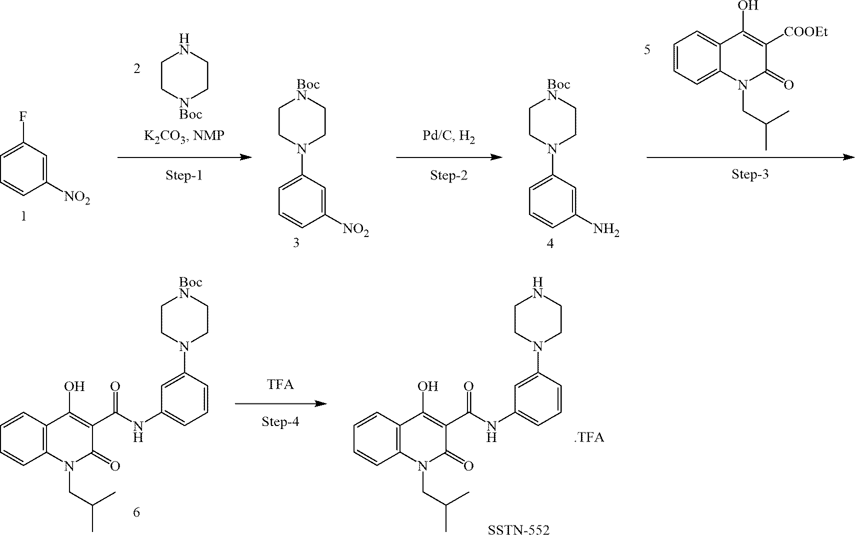

### Step-1: Tert-Butyl 4-(3-Nitrophenyl)Piperazine-1-Carboxylate (3)

To a stirred mixture of 3-fluoro nitrobenzene (1) (100 mg, 0.708 mmol, 1 eq) in NMP (2 mL), tert-butyl piperazine-1-carboxylate (2) (197 mg, 1.06 mmol, 1.5 eq) and potassium carbonate (489 mg, 3.54 mmol, 5 eq) at RT were added. The reaction was subjected to microwave for 1 h at 150° C. The reaction was monitored by TLC (M.Ph: 20% EtOAc in n-hexane). The reaction mixture was quenched with chilled water (50 mL) and extracted with EtOAc (2 × 50 mL). The combined organic layers was washed with brine, dried over sodium sulfate, filtered and concentrated to dryness. The crude was purified through silica gel column chromatography (elution: 0-10% EtOAc in n-hexane) to afford 3 (214 mg, 98.61%) as an off-white solid.

### Step-2: Tert-Butyl 4-(3-Aminophenyl)Piperazine-1-Carboxylate (4)

To a stirred solution of tert-butyl 4-(3-nitrophenyl)piperazine-1-carboxylate (3) (250 mg, 0.813 mmol, 1 eq) in EtOAc (6 mL), MeOH (5 mL) and water (1 mL), 10% Pd/C (25 mg) was added into a hydrogenator. The mixture was degassed for 15 min with the help of alternative vacuum and nitrogen. The reaction was hydrogenated for 3 h at 1 kg/cm² hydrogen pressure. The reaction was monitored by TLC (M.Ph: 50% EtOAc in n-hexane). The mixture was filtered through celite bed and the clear filtrate was concentrated to dryness. The crude was purified through silica gel column chromatography (elution: 100% DCM) to afford 6 (220 mg, 97.7%) as an oily mass. ¹H NMR (DMSO-A, 400 MHz): 8 6.86 (t, J= 8.0 Hz, 1H), 6.14-6.13 (m, 2H), 6.06 (d, J= 8.0 Hz, 1H), 4.87 (bs, 2H), 3.41 (t, J= 4.8 Hz, 4H), 3.16 (t, J= 4.8 Hz, 4H), 1.41-1.38 (m, 9H); LC-MS: m/z 278.32 [M+H]⁺.

### Step-3: Tert-Butyl 4-(3-(4-Hydroxy-1-Isobutyl-2-Oxo-1,2-Dihydroquinoline-3-Carboxamido)Phenyl)Piperazine-1-Carboxylate (6)

To a stirred mixture of ethyl 4-hydroxy-1-isobutyl-2-oxo-1,2-dihydroquinoline-3-carboxylate (5) (145 mg, 0.501 mmol, 1 eq) in DMSO (10 mL), tert-butyl 4-(3-aminophenyl)piperazine-1-carboxylate (4) (230 mg, 0.751 mmol, 1.5 eq) was added. The reaction mixture was stirred at 120° C. for 2 h. The reaction was monitored by TLC (M.Ph: 100% DCM). The reaction mixture was quenched with chilled water (100 mL) and extracted with EtOAc (2 × 100 mL). The combined organic layers was washed with brine, dried over sodium sulfate, filtered and concentrated to dryness. The crude was purified through silica gel column chromatography (elution: 100% DCM) to afford 6 (100 mg, 79.56%) as an off-white solid. ¹H NMR (DMSO-d6, 400 MHz): 8 16.73 (s, 1H), 12.62 (s, 1H), 8.17 (d, J= 8.0 Hz, 1H), 7.85 (t, J= 8.8 Hz, 1H), 7.74 (d, J= 8.8 Hz, 1H), 7.43 (t, J= 7.2 Hz, 1H), 7.27 (t, J= 8.0 Hz, 1H), 7.20-7.16 (m, 2H), 6.81 (d, J= 7.2 Hz, 1H), 4.23 (d, J= 6.8 Hz, 2H), 3.48 (t, J= 4.8 Hz, 4H), 3.17 (t, J= 4.8 Hz, 4H), 2.20-2.16 (m, 1H), 1.46-1.39 (m, 9H), 0.94 (d, J= 7.2 Hz, 6H); LC-MS: m/z 521.4 [M+H]⁺.

### Step-4: 4-Hydroxy-1-Isobutyl-2-Oxo-N-(3-(Piperazin-1-yl)Phenyl)-1,2-Dihydroquinoline-3-Carboxamide Trifluoro Acetic Acid (SSTN-552)

To a stirred mixture of tert-butyl 4-(3-(4-hydroxy-1-isobutyl-2-oxo-1,2-dihydroquinoline-3-carboxamido)phenyl)piperazine-1-carboxylate (6) (100 mg, 0.501 mmol, 1 eq) in DCM (5 mL), trifluoro aceticacid (2 mL) was added at RT at 0° C. The reaction mixture was allowed to RT and stirred at same temperature for 2 h. The reaction was monitored by TLC (M.Ph: 5% MeOH in DCM). The reaction mixture was concentrated to dryness. The crude was triturated in diethyl ether to afford SSTN-552 (98 mg, 98.5%) as an off-white solid. ¹H NMR (DMSO-d6, 400 MHz): 8 16.68 (s, 1H), 12.64 (s, 1H), 8.72 (bs, 2H), 8.17 (d, J= 7.6 Hz, 1H), 7.86 (t, J= 8.0 Hz, 1H), 7.75 (d, J= 8.8 Hz, 1H), 7.44 (t, J= 8.0 Hz, 1H), 7.31-7.22 (m, 3H), 6.86 (d, J= 8.0 Hz, 1H), 4.23 (d, J= 4.8 Hz, 2H), 3.38-3.25 (m, 8H), 2.21-2.15 (m, 1H), 0.94 (d, J= 6.0 Hz, 6H); LC-MS: m/z 421.15 [M+H]⁺; HPLC: 99.65%.

SSTN-554

Synthesis of 4-hydroxy-1-isobutyl-N-(3-morpholinophenyl)-2-oxo-1,2-dihydroquinoline-3-carboxamide (SSTN-554)

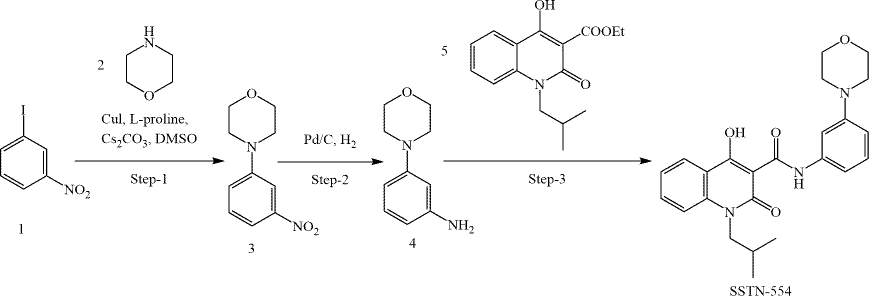

### Step-1: 4-(3-Nitrophenyl)Morpholine (3)

To a stirred mixture of 3-iodo nitrobenzene (1) (500 mg, 2.0 mmol, 1 eq) in DMSO (15 mL), morpholine (2) (262 mg, 3.01 mmol, 1.5 eq), cesium carbonate (1.3 g, 4.0 mmol, 2 eq), CuI (457 mg, 2.4 mmol, 1.2 eq) and L-proline (460 mg, 4 mmol, 2 eq) were added at RT. The mixture was degassed for 15 min with the help of alternative vacuum and nitrogen. The reaction was stirred at 120° C. for 16 h. The reaction was monitored by TLC (M.Ph: 20% EtOAc in n-hexane). The reaction mixture was quenched with chilled water (250 mL) and extracted with EtOAc (2 × 200 mL). The combined organic layers was washed with brine, dried over sodium sulfate, filtered and concentrated to dryness. The crude was purified through silica gel column chromatography (elution: 0-20% EtOAc in n-hexane) to afford 3 (160 mg, 38.2%) as yellow solid. ¹H NMR (DMSO-d6, 400 MHz): 8 7.75-7.72 (m, 2H), 7.45 (t, J= 8.4 Hz, 1H), 7.24 (d, J= 8.4 Hz, 1H), 3.93 (t, J= 4.4 Hz, 4H), 3.29 (t, J= 4.4 Hz, 4H); LC-MS: m/z 209.05 [M+H]⁺.

### Step-2: 4-(3-Nitrophenyl)Morpholine (4)

To a stirred solution of 4-(3-nitrophenyl)morpholine (3) (150 mg, 0.72 mmol, 1 eq) in EtOAc (5 mL), MeOH (4 mL) and water (1 mL), 10% Pd/C (25 mg) was added into a hydrogenator. The mixture was degassed for 15 min with the help of alternative vacuum and nitrogen. The reaction was hydrogenated for 3 h at 1 kg/cm² hydrogen pressure. The reaction was monitored by TLC (M.Ph: 50% EtOAc in n-hexane). The mixture was filtered through celite bed and the clear filtrate was concentrated to dryness. The crude was purified through silica gel column chromatography (elution: 100% DCM) to afford 4 (110 mg, 85.9%) as an oily mass. ¹H NMR (DMSO-d6, 400 MHz): 8 6.86 (t, J= 8.4 Hz, 1H), 6.12-6.10 (m, 2H), 7.05 (d, J= 7.6 Hz, 1H), 4.86 (bs, 2H), 3.70 (t, J= 5.2 Hz, 4H), 2.99 (t, J= 5.2 Hz, 4H); LC-MS: m/z 178.90 [M+H]⁺.

### Step-3: 4-Hydroxy-1-Isobutyl-N-(3-Morpholinophenyl)-2-Oxo-1,2-Dihydroquinoline-3-Carboxamide (SSTN-554)

to a stirred mixture of ethyl 4-hydroxy-1-isobutyl-2-oxo-1,2-dihydroquinoline-3-carboxylate (5) (110 mg, 0.38 mmol, 1 eq) in dmso (3 ml), 4-(3-nitrophenyl)morpholine (4) (101 mg, 0.57 mmol, 1.5 eq) was added. the reaction mixture was stirred at 120° C. for 2 h. the reaction was monitored by tlc (m.ph: 100% dcm). the reaction mixture was quenched with chilled water (100 ml) and extracted with etoac (2 × 100 ml). the combined organic layers was washed with brine, dried over sodium sulfate, filtered and concentrated to dryness. the crude was purified through silica gel column chromatography (elution: 100% dcm) to afford sstn-554 (38 mg, 23.44%) as an off-white solid. ¹h nmr (dmso-d6, 400 mhz): 8 16.71 (s, 1h), 12.60 (s, 1h), 8.15 (d, j= 8.4 hz, 1h), 7.83 (t, j= 7.2 hz, 1h), 7.72 (d, j= 8.4 hz, 1h), 7.41 (t, j= 7.2 hz, 1h), 7.26 (t, j= 8.0 hz, 1h), 7.18-7.13 (m, 2h), 6.79 (d, j= 6.8 hz, 1h), 4.19 (d, j= 5.6 hz, 2h), 3.74 (t, j= 4.8 hz, 4h), 3.13 (t, j= 4.8 hz, 4h), 2.18-2.15 (m, 1h), 0.92 (d, j= 6.4 hz, 6h); lc-ms: m/z 422.10 [m+h]⁺; hplc: 98.05%.

SSTN-560 (free base, HCl salt, formate)

Synthesis of 4-hydroxy-1-isobutyl-N-(3-(4-methylpiperazin-1-yl)phenyl)-2-oxo-1,2-dihydroquinoline-3-carboxamide (SSTN-560, Notch1 Reporter Assay IC₅₀: 14.2 µM)

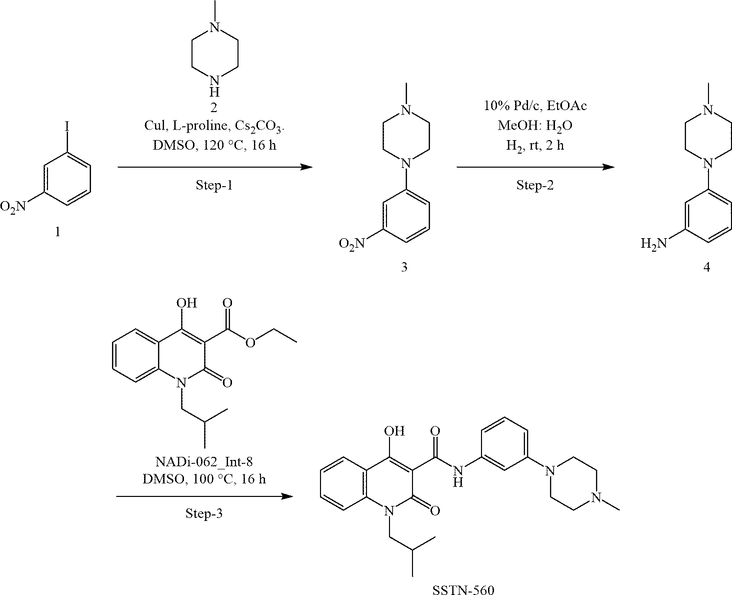

### Step-1: 1-Methyl-4-(3-Nitrophenyl)Piperazine (3)

To a stirred mixture of 1-iodo-3-nitrobenzene (1) (500 mg, 2.007 mmol, 1 eq) and 1-methylpiperazine (2) (301 mg, 3.01 mmol, 1.5 eq) in DMSO (15 mL) were added CuI (687 mg, 3.61 mmol, 1.2 eq), L-proline (462 mg, 4.01 mmol, 2 eq) and cesium carbonate (1.30 g, 4.01 mmol, 2 eq) at room temperature. The reaction mixture was heated at 120° C. for 16 h. The reaction was monitored by TLC (M.Ph: 5% Methanol in DCM). The reaction mixture was cooled to room temperature and filtered through a Celite bed. The filtrate was diluted with ethyl acetate and washed with water (2 × 50 mL) followed by brine (50 mL). The organic layer was dried over anhydrous sodium sulfate, filtered and concentrated in vacuo resulting in the crude compound. The crude compound was purified through 230-400 mesh size silica gel column chromatography (elution: 0-5% methanol in DCM) to afford 3 (155 mg, 34.9%) as yellow solid. ¹H NMR (DMSO-A, 400 MHz): 8 ppm 7.68 (s, 1H), 7.62 (d, J=7.83 Hz, 1H), 7.48-7.55 (m, 1H), 7.42-7.47 (m, 1H), 3.27-3.33 (m, 4H), 2.47-2.52 (m, 4H), 2.27 (s, 3H); LC-MS: m/z 221.80 [M+H]⁺.

### Step-2: 3-(4-Methylpiperazin-1-yl)Aniline (4)

To a stirred solution of 1-methyl-4-(3-nitrophenyl)piperazine (3) (150 mg, 0.677 mmol, 1 eq) in MeOH (4 mL) was added solution of 10% Pd/C (15 mg) in EtOAc (5 mL) followed by water (1 mL) into a hydrogenator. The mixture was degassed for 15 min with the help of alternative vacuum and nitrogen. The reaction was hydrogenated for 2 h at 1 kg/cm² hydrogen pressure. The reaction was monitored by TLC (M.Ph: 5% Methanol in DCM). The reaction mixture was filtered through Celite bed and the filtrate was concentrated in vacuo resulting in the crude compound. The crude compound was purified by trituration with diethyl ether and hexane, filtered and dried in vacuum to afford 4 (110 mg, 85.2%). ¹H NMR (DMSO-d6, 400 MHz): 8 ppm 6.92 (t, J=8.07 Hz, 1H), 6.23 (br. s, 1H), 6.20 (d, J=7.82 Hz, 1H), 6.11 (d, J=7.34 Hz, 1H), 4.92 (br. s, 2H), 3.07-3.14 (m, 4H), 2.48-2.53 (m, 4H), 2.29 (s, 3H); LC-MS: m/z 192.10 [M+H]⁺.

### Step-3: 4-Hydroxy-1-Isobutyl-N-(3-(4-Methylpiperazin-1-Yl)Phenyl)-2-Oxo-1,2-Dihydroquinoline-3-Carboxamide (SSTN-560)

To a stirred mixture of ethyl 4-hydroxy-1-isobutyl-2-oxo-1,2-dihydroquinoline-3-carboxylate (110 mg, 0.380 mmol, 1 eq) in DMSO (3 mL) was added 3-(4-methylpiperazin-1-yl)aniline (4) (109 mg, 0.570 mmol, 1.5 eq) at room temperature. The reaction mixture was heated at 100° C. for 16 h. The progress of the reaction was monitored by TLC (M.Ph: 5% Methanol in DCM). The reaction mixture was diluted with EtOAc (50 mL). The organic layer was washed with ice cold water (2 × 50 mL) followed by brine (50 mL). The organic layer was separated, dried over anhydrous sodium sulfate, filtered and concentrated in vacuo resulting in the crude compound. The crude compound was purified through 230-400 mesh size silica gel column chromatography (elution: 0-5% methanol in DCM) to afford SSTN-560 (30 mg, 18.1%) as an off-white solid. ¹H NMR (DMSO-A, 400 MHz): 8 ppm 16.73 (br. s, 1H), 12.63 (br. s, 1H), 8.16 (d, J=8.31 Hz, 1H), 7.80-7.88 (m, 1H), 7.74 (d, J=8.80 Hz, 1H), 7.42 (t, J=7.34 Hz, 1H), 7.22-7.27 (m, 1H), 7.19 (br. s, 1H), 7.15 (d, J=7.82 Hz, 1H), 6.80 (d, J=8.31 Hz, 1H), 4.22 (d, J=6.85 Hz, 2H), 3.15-3.24 (m, 4H), 2.54-2.64 (m, 4H), 2.32 (br. s, 3H), 2.17-2.22 (m, 1H), 0.94 (d, J=6.85 Hz, 6H); LC-MS: m/z 435.10 [M+H]⁺; HPLC: 96.52%

### Step-3: 4-Hydroxy-1-Isobutyl-N-(3-(4-Methylpiperazin-1-Yl)Phenyl)-2-Oxo-1,2-Dihydroquinoline-3-Carboxamide Formate Salt (SSTN-560 Formate Salt, Notch1 Reporter Assay IC₅₀: 10.2 µM)

A solution of ethyl 4-hydroxy-1-isobutyl-2-oxo-1,2-dihydroquinoline-3-carboxylate (300 mg, 1.037 mmol, 1 eq) and 3-(4-methylpiperazin-1-yl)aniline (4) (238 mg, 1.245 mmol, 1.2 eq) in DMSO (5 mL) was heated at 100° C. for 16 h in a sealed vessel. The progress of the reaction was monitored by TLC (M.Ph: 5% Methanol in DCM). The reaction mixture was diluted with ice cold water (50 mL) and extracted with EtOAc (2 × 100 mL). The combined organic layer was washed with water (2 × 50 mL) followed by brine (20 mL). The organic layer was separated, dried over anhydrous sodium sulfate, filtered and concentrated in vacuo resulting in the crude compound. The crude compound was purified through 230-400 mesh size silica gel column chromatography (elution: 2-8% methanol in DCM). The column purified compound was repurified by preparative HPLC to afford SSTN-560 _Formate salt (170 mg, 34.1%) as an off white solid. ¹H NMR (DMSO-d6, 400 MHz): 8 ppm 12.62 (br. s, 1H), 8.13-8.19 (m, 2H), 7.79-7.87 (m, 1H), 7.73 (d, J--8.31 Hz, 1H), 7.41 (t, J--7.34 Hz, 1H), 7.21-7.27 (m, 1H), 7.19 (br. s, 1H), 7.12 (d, J--7.34 Hz, 1H), 6.79 (d, .T--6.85 Hz, 1H), 4.17-4.26 (m, 2H), 3.13-3.27 (m, 8H), 2.24 (br. s, 3H), 2.13-2.20 (m, 1H), 0.93 (d, J--5.87 Hz, 6H); LC-MS: m/z 435.30 [M+H]⁺; HPLC: 99.56%

Synthesis of 4-hydroxy-1-isobutyl-N-(3-(4-methylpiperazin-1-yl)phenyl)-2-oxo-1,2-dihydroquinoline-3-carboxamide (SSTN-560_HCI salt, Notch1 Reporter Assay IC₅₀: 7.7 µM)

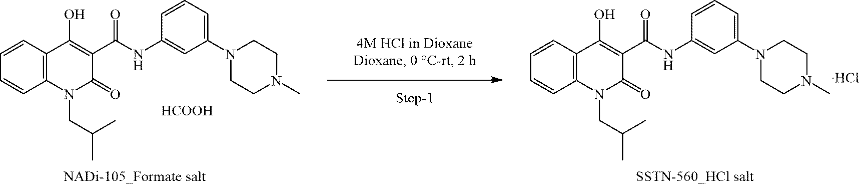

### Step-1: 4-Hydroxy-1-Isobutyl-N-(3-(4-Methylpiperazin-1-Yl)Phenyl)-2-Oxo-1,2-Dihydroquinoline-3-Carboxamide (SSTN-560_Hc1 Salt)

To a stirred mixture of 4-hydroxy-1-isobutyl-N (3-(4-methylpiperazin-1-yl)phenyl)-2-oxo-1,2-dihydroquinoline-3-carboxamide formate salt (SSTN-560 Formate salt) (100 mg, 0.208 mmol, 1 eq) in dioxane (5 mL) at 0° C. was added 4 M HCl in dioxane (1 mL). The reaction mixture was allowed to attain at room temperature and stirred for 2 h. The progress of the reaction was monitored by TLC (M.Ph: 5% methanol in DCM). The reaction mixture was concentrated in vacuo resulting in the crude compound. The crude compound was purified by trituration with diethyl ether (5 mL) and n-hexane (5 mL), filtered and dried under vacuum to afford SSTN-560_HCI salt (80 mg, 81.6%) as an off-white solid. ¹H NMR (DMSO-d6, 400 MHz): 8 ppm 16.69 (s, 1H), 12.64 (br. s, 1H), 10.36 (br. s, 1H), 8.16 (d, J=7.34 Hz, 1H), 7.81-7.88 (m, 1H), 7.74 (d, J--8.80 Hz, 1H), 7.42 (t, .T--7.34 Hz, 1H), 7.23-7.34 (m, 3H), 6.86 (d, J--7.34 Hz, 1H), 4.23 (d, J--3.91 Hz, 2H), 3.88 (d, J--11.74 Hz, 2H), 3.51 (d, J--11.25 Hz, 2H), 3.04-3.22 (m, 4H), 2.84 (d, J--3.91 Hz, 3H), 2.14-2.24 (m, 1H), 0.94 (d, J--6.36 Hz, 6H); LC-MS: m/z 435.10 [M+H]⁺; HPLC: 99.78%

SSTN-561

Synthesis ofN-(3-(cyclopropyl(methyl)amino)phenyl)-4-hydroxy-1-isobutyl-2-oxo-1,2-dihydroquinoline-3 -carboxamide (SSTN-561)

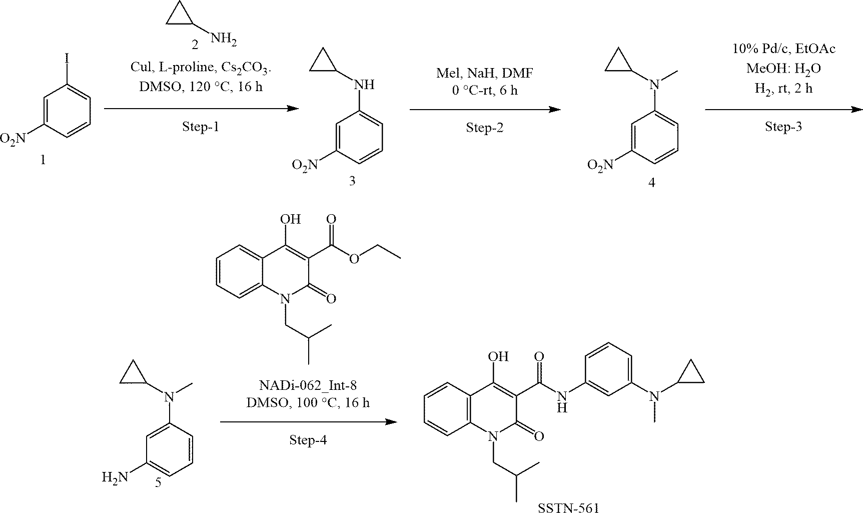

### Step-1: N-Cyclopropyl-3-Nitroaniline (3)

To a stirred mixture of 1-iodo-3-nitrobenzene (1) (800 mg, 3.212 mmol, 1 eq) and cyclopropanamine (2) (274 mg, 4.819 mmol, 1.5 eq) in DMSO (15 mL) were added CuI (734 mg, 3.855 mmol, 1.2 eq), L-proline (739 mg, 6.425 mmol, 2 eq) and cesium carbonate (2.08 g, 6.425 mmol, 2 eq) at room temperature. The reaction mixture was heated at 120° C. for 16 h in a sealed tube. The reaction was monitored by TLC (M.Ph: 20% ethyl acetate in n-hexane). The reaction mixture was poured over ice cold water and stirred for 10 min, and diluted with ethyl acetate (200 mL) and stirred for another 10 min. The resulting solution was filtered through a Celite bed. The organic layer from the filtrate was separated and washed with water (2 × 50 mL) followed by brine (50 mL). The organic layer was dried over anhydrous sodium sulfate, filtered and concentrated in vacuo resulting in the crude compound. The crude compound was purified through 100-200 mesh size silica gel column chromatography (elution: 0-20% ethyl acetate in n-hexane) to afford 3 (225 mg, 39.3%) as brown oil. ¹H NMR (DMSO-d6, 400 MHz): 8 ppm 7.56 (t, J=1.96 Hz, 1H), 7.41-7.50 (m, 2H), 7.17 (d, J=7.34 Hz, 1H), 6.91 (br. s, 1H), 2.47-2.53 (m, 1H), 0.80-0.89 (m, 2H), 0.47-0.53 (m, 2H); LC-MS: m/z 179.17 [M+H]⁺.

### Step-2: N-Cyclopropyl-N-Methyl-3-Nitroaniline (4)

To a stirred mixture of N-cyclopropyl-3-nitroaniline (3) (160 mg, 0.897 mmol, 1 eq) in DMF (3 mL) at 0° C. was added sodium hydride (60% dispersion in oil, 43 mg, 1.077 mmol, 1.2 eq) slowly and stirred for 10-15 min. To the resulting solution was added methyl iodide (0.055 mL, 0.897 mmol, 1 eq) and further stirred at room temperature for 6 h. The reaction was monitored by TLC (M.Ph: 20% ethyl acetate in n-hexane). The reaction mixture was poured over ice cold water (100 mL) and extracted with ethyl acetate (2 × 50 mL). The combined organic layer was washed with water (2 × 50 mL) followed by brine (50 mL). The organic layer was separated, dried over anhydrous sodium sulfate, filtered and concentrated in vacuo resulting in the crude compound. The crude compound from batch no. E20068-020 (50 mg scale) was combined after work-up for purification with batch no. E20068-021 (160 mg scale). The crude compound from both batches were combined purified through 100-200 mesh size silica gel column chromatography (elution: 0-20% ethyl acetate in n-hexane) to afford 4 (156 mg, 69%) as yellow solid. ¹H NMR (DMSO-A, 400 MHz): 8 ppm 7.67 (br. s, 1H), 7.50-7.54 (m, 1H), 7.45 (t, J=8.07 Hz, 1H), 7.32-7.36 (m, 1H), 3.00 (s, 3H), 2.52-2.56 (m, 1H), 0.86-0.94 (m, 2H), 0.56-0.62 (m, 2H); LC-MS: m/z 193.05 [M+H]⁺.

### Step-3: N1-Cyclopropyl-N1-Methylbenzene-1,3-Diamine (5)

To a stirred solution of N-cyclopropyl-N-methyl-3-nitroaniline (4) (150 mg, 0.780 mmol, 1 eq) in MeOH (3 mL) was added solution of 10% Pd/C (15 mg) in EtOAc (5 mL) followed by water (1 mL) into a hydrogenator. The mixture was degassed for 15 min with the help of alternative vacuum and nitrogen. The reaction was hydrogenated for 2 h at 1 kg/cm² hydrogen pressure. The reaction was monitored by TLC (M.Ph: 5% Methanol in DCM). The reaction mixture was filtered through Celite bed and the filtrate was concentrated in vacuo resulting in the crude compound. The crude compound was purified by trituration with diethyl ether and hexane, filtered and dried in vacuum to afford 5 (126 mg, 99.5%). ¹H NMR (DMSO-d6, 400 MHz): 8 ppm 6.81 (t, J=7.82 Hz, 1H), 6.21 (s, 1H), 6.16 (d, J=7.82 Hz, 1H), 5.96 (d, J=7.82 Hz, 1H), 4.79 (s, 2H), 2.82 (s, 3H), 2.21-2.29 (m, 1H), 0.72-0.79 (m, 2H), 0.46-0.51 (m, 2H); LC-MS: m/z 163.10 [M+H]⁺.

### Step-4: N-(3-(Cyclopropyl(Methyl)Amino)Phenyl)-4-Hydroxy-1-Isobutyl-2-Oxo-1,2-Dihydroquinoline-3-Carboxamide (SSTN-561)

To a stirred mixture of ethyl 4-hydroxy-1-isobutyl-2-oxo-1,2-dihydroquinoline-3-carboxylate (140 mg, 0.483 mmol, 1 eq) in DMSO (3 mL) was added N1-cyclopropyl-N1-methylbenzene-1,3-diamine (5) (117 mg, 0.725 mmol, 1.5 eq) at room temperature. The reaction mixture was heated at 100° C. for 16 h. The progress of the reaction was monitored by TLC (M.Ph: 5% Methanol in DCM). The reaction mixture was poured into ice cold water (50 mL) and extracted with EtOAc (50 mL). The organic layer was washed with water (2 × 50 mL) followed by brine (50 mL). The organic layer was separated, dried over anhydrous sodium sulfate, filtered and concentrated in vacuo resulting in the crude compound. The crude compound was purified through 230-400 mesh size silica gel column chromatography (elution: 0-5% methanol in DCM) to afford SSTN-561 (100 mg, 51.2%) as an off white solid. ¹H NMR (DMSO-d6, 400 MHz): 8 ppm 16.84 (s, 1H), 12.59 (s, 1H), 8.16 (d, J=7.82 Hz, 1H), 7.81-7.86 (m, 1H), 7.73 (d, J=8.80 Hz, 1H), 7.41 (t, J=7.34 Hz, 1H), 7.27 (s, 1H), 7.22 (t, J=8.31 Hz, 1H), 7.02 (d, J=8.31 Hz, 1H), 6.77-6.83 (m, 1H), 4.22 (d, J=5.87 Hz, 2H), 2.95 (s, 3H), 2.43 (td, J=3.18, 6.36 Hz, 1H), 2.18 (td, J=6.60, 13.20 Hz, 1H), 0.94 (d, J=6.36 Hz, 6H), 0.82-0.88 (m, 2H), 0.54-0.60 (m, 2H); LC-MS: m/z 406.15 [M+H]⁺; HPLC: 98.99%

SSTN-562

Synthesis ofN-(3-((cyclopropylmethyl)(methyl)amino)phenyl)-4-hydroxy-1-isobutyl-2-oxo-1,2-dihydroquinoline-3 -carboxamide (SSTN-562)

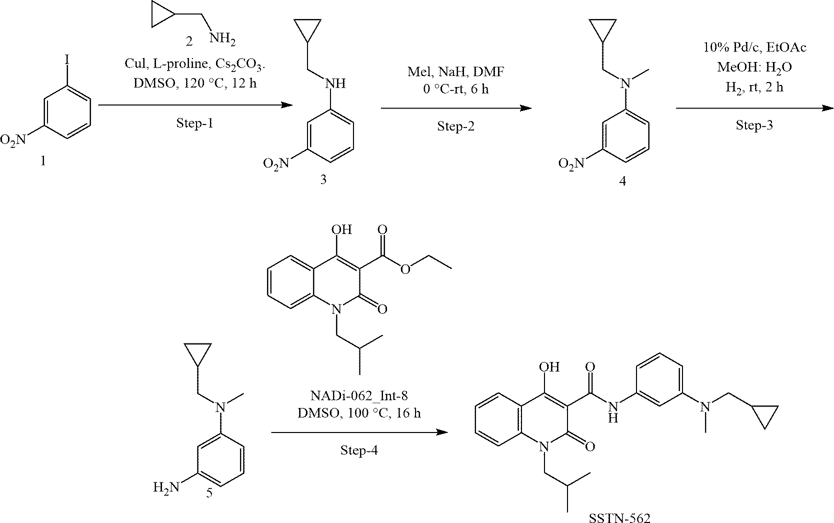

### Step-1: N-(Cyclopropylmethyl)-3-Nitroaniline (3)

To a stirred mixture of 1-iodo-3-nitrobenzene (1) (1.00 g, 4.015 mmol, 1 eq) and cyclopropylmethanamine (2) (428 mg, 6.023 mmol, 1.5 eq) in DMSO (10 mL) were added CuI (0.917 mg, 4.819 mmol, 1.2 eq), L-proline (924 mg, 8.031 mmol, 2 eq) and cesium carbonate (2.60 g, 8.031 mmol, 2 eq) at room temperature. The reaction mixture was heated at 120° C. for 12 h in a sealed tube. The reaction was monitored by TLC (M.Ph: 20% ethyl acetate in n-hexane). The reaction mixture was poured over ice cold water and stirred for 10 min, and diluted with ethyl acetate (200 mL) and stirred for another 10 min. The resulting solution was filtered through a celite bed. The organic layer from the filtrate was separated and washed with water (2 × 100 mL) followed by brine (2 × 100 mL). The organic layer was dried over anhydrous sodium sulfate, filtered and concentrated in vacuo resulting in the crude compound. The crude compound was purified through 100-200 mesh size silica gel column chromatography (elution: 0-20% ethyl acetate in n-hexane) to afford 3 (260 mg, 33.7%) as brown oil. ¹H NMR (DMSO-d6, 400 MHz): 8 ppm 7.27-7.35 (m, 3H), 6.99 (d, J=3.42 Hz, 1H), 6.46 (br. s, 1H), 2.95 (t, J=5.87 Hz, 2H), 0.99-1.11 (m, 1H), 0.45-0.53 (m, 2H), 0.23 (d, J=₄.40 Hz, 2H); LC-MS: m/z 192.90 [M+H]⁺.

### Step-2: N-(Cyclopropylmethyl)-N-Methyl-3-Nitroaniline (4)

To a stirred mixture of N (cyclopropylmethyl)-3-nitroaniline (3) (250 mg, 1.300 mmol, 1 eq) in DMF (3 mL) at 0° C. was added sodium hydride (60% dispersion in oil, 62 mg, 1.560 mmol, 1.2 eq) slowly and stirred for 10-15 min. To the resulting solution was added methyl iodide (0.081 mL, 1.300 mmol, 1 eq) and further stirred at room temperature for 6 h. The reaction was monitored by TLC (M.Ph: 20% ethyl acetate in n-hexane). The reaction mixture was poured over ice cold water (100 mL) and extracted with ethyl acetate (2 × 50 mL). The combined organic layer was washed with water (2 × 50 mL) followed by brine (50 mL). The organic layer was separated, dried over anhydrous sodium sulfate, filtered and concentrated in vacuo resulting in the crude compound. The crude compound was purified through 100-200 mesh size silica gel column chromatography (elution: 0-20% ethyl acetate in n-hexane) to afford 4 (175 mg, 65.2%) as yellow solid. LC-MS: m/z 207.40 [M+H]⁺.

### Step-3: N1-(Cyclopropylmethyl)-N1-Methylbenzene-1,3-Diamine (5)

To a stirred solution of N-(cyclopropylmethyl)-N-methyl-3-nitroaniline (4) (175 mg, 0.848 mmol, 1 eq) in MeOH (3 mL) was added solution of 10% Pd/C (15 mg) in EtOAc (5 mL) followed by water (1 mL) into a hydrogenator. The mixture was degassed for 15 min with the help of alternative vacuum and nitrogen. The reaction was hydrogenated for 2 h at 1 kg/cm² hydrogen pressure. The reaction was monitored by TLC (M.Ph: 5% Methanol in DCM). The reaction mixture was filtered through Celite bed and the filtrate was concentrated in vacuo resulting in the crude compound. The crude compound was purified by trituration with diethyl ether and hexane, filtered and dried in vacuum to afford 5 (146 mg, 97.6%). ¹H NMR (DMSO-d6, 400 MHz): 8 ppm 6.79 (t, J=7.83 Hz, 1H), 5.98-6.00 (m, 1H), 5.95 (dd, J=1.96, 8.31 Hz, 1H), 5.88 (dd, J=1.47, 7.82 Hz, 1H), 4.77 (s, 2H), 3.10 (d, J=6.36 Hz, 2H), 2.83 (s, 3H), 0.89-0.99 (m, 1H), 0.37-0.45 (m, 2H), 0.16-0.22 (m, 2H); LC-MS: m/z 177.00 [M+H]⁺.

### Step-4: N-(3-((Cyclopropylmethyl)(Methyl)Amino)Phenyl)-4-Hydroxy-1-Isobutyl-2-Oxo-1,2-Dihydroquinoline-3-Carboxamide (SSTN-562)

To a stirred mixture of ethyl 4-hydroxy-1-isobutyl-2-oxo-1,2-dihydroquinoline-3-carboxylate (160 mg, 0.553 mmol, 1 eq) in DMSO (3 mL) was added N1-(cyclopropylmethyl)-M -methylbenzene-1,3-diamine (5) (146 mg, 0.829 mmol, 1.5 eq) at room temperature. The reaction mixture was heated at 100° C. for 16 h. The progress of the reaction was monitored by TLC (M.Ph: 5% Methanol in DCM). The reaction mixture was poured into ice cold water (50 mL) and extracted with EtOAc (50 mL). The organic layer was washed with water (2 x× 50 mL) followed by brine (50 mL). The organic layer was separated, dried over anhydrous sodium sulfate, filtered and concentrated in vacuo resulting in the crude compound. The crude compound was purified through 230-400 mesh size silica gel column chromatography (elution: 0-2% methanol in DCM) to afford SSTN-562 (23 mg, 9.90%) as an off white solid. ¹H NMR (DMSO-d6, 400 MHz): 8 ppm 16.84 (br. s, 1H), 12.58 (br. s, 1H), 8.16 (d, J=7.82 Hz, 1H), 7.65-7.89 (m, 2H), 7.40 (br. s, 1H), 7.13-7.21 (m, 1H), 7.01-7.08 (m, 1H), 6.93 (d, J=7.82 Hz, 1H), 6.54-6.64 (m, 1H), 4.21 (br. s, 2H), 3.24 (d, J=6.36 Hz, 2H), 2.95 (s, 3H), 2.12-2.22 (m, 1H), 0.96-1.04 (m, 1H), 0.93 (d, J=6.36 Hz, 6H), 0.46 (d, J=7.83 Hz, 2H), 0.25 (d, J=4.40 Hz, 2H). LC-MS: m/z 420.30 [M+H]⁺; HPLC: 98.70%

SSTN-563

Synthesis of 5-bromo-4-hydroxy-1-isobutyl-N-(3-methylpyridin-2-yl)-2-oxo-1,2-dihydroquinoline-3-carboxamide (SSTN-563)

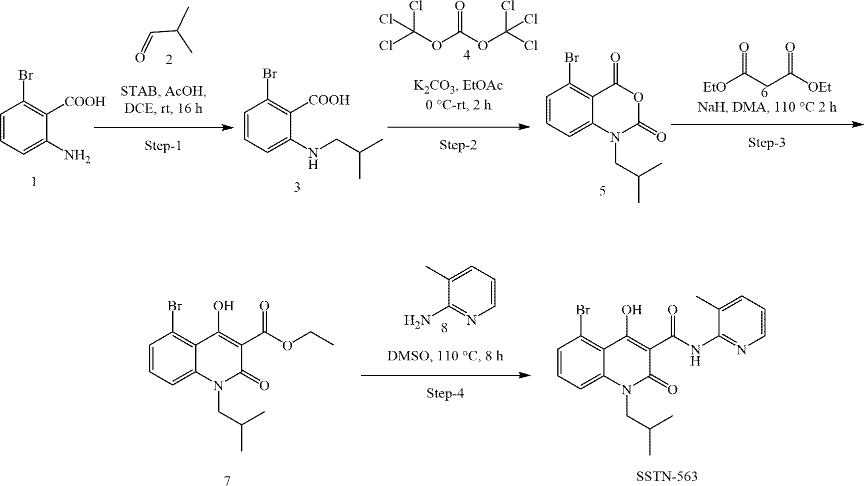

### Step-1: 2-Bromo-6-(Isobutylamino)Benzoic Acid (3)

To a stirred mixture of 2-amino-6-bromobenzoic acid (1) (10.0 g, 46.28 mmol, 1 eq) in DCE (400 mL) was added isobutyraldehyde (2) (3.67 g, 50.91 mmol, 1.1 eq) at room temperature. To the resulting solution at 0° C. was added STAB (39.2 g, 185.15 mmol, 4 eq) followed by acetic acid (13.2 mL, 231.44 mmol, 5 eq) and stirred for 5 min. The reaction mixture was allowed to attain room temperature and stirred for 16 h. The reaction was monitored by TLC (M.Ph: 5% methanol in DCM). The reaction mixture was concentrated in vacuo upto dryness. The crude residue obtained was dissolved in DCM (500 mL) and washed with saturated NaHC03 solution (500 mL) followed by water (500 mL). The combined organic layer was dried over anhydrous sodium sulfate, filtered and concentrated in vacuo resulting in the crude compound. The crude compound was purified through 100-200 mesh size silica gel column chromatography (elution: 0-1% methanol in DCM) to afford 3 (8.12 g, 64.9%) as brown solid. Note: Two batches on 5 g scale were performed. ¹H NMR (DMSO-d₆, 400 MHz): 8 ppm 13.15 (br. s, 1H), 7.09 (t, J=8.07 Hz, 1H), 6.80 (d, J=7.34 Hz, 1H), 6.67 (d, J=8.31 Hz, 1H), 2.91 (d, J=6.85 Hz, 2H), 1.84 (td, J=6.79, 13.33 Hz, 1H), 0.90 (d, J=6.36 Hz, 6H); LC-MS: m/z 271.90 [M+H]⁺.

### Step-2: 5-Bromo-1-Isobutyl-2H-Benzo[d][1,3]Oxazine-2,4(1H)-Dione (5)

To a stirred mixture of 2-bromo-6-(isobutylamino)benzoic acid (3) (8.00 g, 29.51 mmol, 1 eq) in ethyl acetate (300 mL) at 0° C. was added triphosgene (4) (4.30 g, 14.75 mmol, 0.5 eq) and potassium carbonate (6.10 g, 44.27 mmol, 1.2 eq) and stirred for 5 min. The reaction mixture was allowed to attain room temperature and stirred for 2 h. The reaction was monitored by TLC (M.Ph: 20% ethyl acetate in n-hexane). The reaction mixture was poured over ice cold water (100 mL) and extracted with ethyl acetate (2 × 100 mL). The combined organic layer was washed with water (2 × 300 mL) followed by brine (200 mL). The organic layer was separated, dried over anhydrous sodium sulfate, filtered and concentrated in vacuo to afford 5 (8.10 g, 92%) as yellow solid. ¹H NMR (DMSO-d₆, 400 MHz): δ ppm 7.58-7.69 (m, 2H), 7.49 (d, J=7.82 Hz, 1H), 3.86 (d, J=7.34 Hz, 2H), 2.07 (td, J=6.66, 13.57 Hz, 1H), 0.95 (d, J=6.85 Hz, 6H); LC-MS: m/z 299.80 [M+H]⁺.

### Step-3: Ethyl 5-Bromo-4-Hydroxy-1-Isobutyl-2-Oxo-1,2-Dihydroquinoline-3-Carboxylate (7)

To a stirred solution of 5-bromo-1-isobutyl-2H-benzo[d][1,3]oxazine-2,4(1H)-dione (5) (4.00 g, 13.41 mmol, 1 eq) and diethyl malonate (6) (2.79 g, 17.44 mmol, 1.3 eq) in DMA (50 mL) at 0° C. was added sodium hydride (60% dispersion in oil, 804 mg, 20.12 mmol, 1.5 eq) under nitrogen atmosphere. The reaction mixture was heated at 110° C. for 2 h. The reaction was monitored by TLC (M.Ph: 30% ethyl acetate in n-hexane). The reaction mixture was poured over ice cold water and acidified with 1N HCl solution and extracted with ethyl acetate (3 × 50 mL). The organic layer was separated, dried over anhydrous sodium sulfate, filtered and concentrated in vacuo resulting in the crude compound. The crude compound was purified by 100-200 mesh size silica gel column chromatography (elution: 0-30% ethyl acetate in n-hexane) to afford 7 (4.20 g, 85.02%). ¹H NMR (DMSO-d₆, 400 MHz): δ ppm 13.93 (br. s, 1H), 7.50-7.62 (m, 3H), 4.34 (q, J=7.12 Hz, 2H), 4.09 (d, J=5.59 Hz, 2H), 2.07 (td, J=6.83, 13.54 Hz, 1H), 1.31 (t, J=7.12 Hz, 3H), 0.87 (d, J=6.61 Hz, 6H); LC-MS: m/z 367.80 [M+H]⁺.

### Step-4: 5-Bromo-4-Hydroxy-1-Isobutyl-N-(3-Methylpyridin-2-yl)-2-Oxo-1,2-Dihydroquinoline-3-Carboxamide (SSTN-563)

To a stirred mixture of ethyl 5-bromo-4-hydroxy-1-isobutyl-2-oxo-1,2-dihydroquinoline-3-carboxylate (7) (1.00 g, 2.715 mmol, 1 eq) in DMSO (10 mL) was added 3-methylpyridin-2-amine (8) (352 mg, 3.258 mmol, 1.2 eq) at room temperature. The reaction mixture was heated at 110° C. for 8 h. The progress of the reaction was monitored by TLC (M.Ph: 5% Methanol in DCM). The reaction mixture was poured into ice cold water (50 mL) and extracted with EtOAc (100 mL). The organic layer was washed with water (2 ×100 mL) followed by brine (2 × 100 mL). The organic layer was separated, dried over anhydrous sodium sulfate, filtered and concentrated in vacuo resulting in the crude compound. The crude compound was purified through 100-200 mesh size silica gel column chromatography (elution: 0-1% methanol in DCM) to afford SSTN-563 (360 mg, 32.7%) as white solid. ¹H NMR (DMSO-d₆, 400 MHz): δ ppm 17.75 (br. s, 1H), 12.61 (br. s, 1H), 8.30-8.36 (m, 1H), 7.77 (dd, J=8.01, 13.86 Hz, 2H), 7.58-7.70 (m, 2H), 7.30 (dd, J=4.83, 7.63 Hz, 1H), 4.20-4.29 (m, 2H), 2.29 (s, 3H), 2.10-2.20 (m, 1H), 0.93 (d, J=6.61 Hz, 6H); LC-MS: m/z 429.90 [M+H]⁺; HPLC: 98.78%.

SSTN-564

Synthesis of 7-bromo-4-hydroxy-1-isobutyl-N-(3-methylpyridin-2-yl)-2-oxo-1,2-dihydroquinoline-3-carboxamide (SSTN-564)

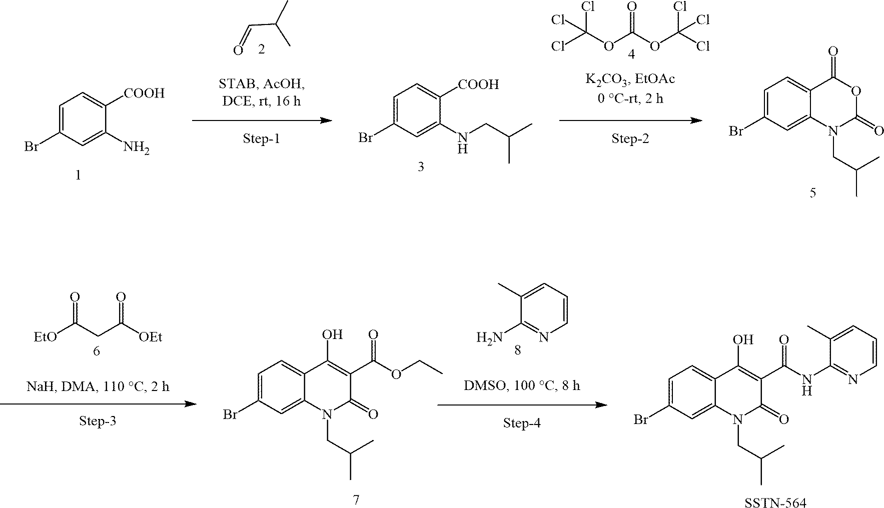

### Step-1: 4-Bromo-2-(Isobutylamino)Benzoic Acid (3)

To a stirred mixture of 2-amino-4-bromobenzoic acid (1) (500 mg, 2.314 mmol, 1 eq) in DCE (25 mL) was added isobutyraldehyde (2) (183 mg, 2.546 mmol, 1.1 eq) and STAB (1.96 g, 9.259 mmol, 4 eq) followed by acetic acid (0.69 mL, 11.57 mmol, 5 eq) at room temperature. The reaction mixture was stirred at room temperature for 16 h. The reaction was monitored by TLC (M.Ph: 5% methanol in DCM). The reaction mixture was diluted with DCM (50 mL) and washed with water (50 mL) followed by brine (50 mL). The organic layer was separated, dried over anhydrous sodium sulfate, filtered and concentrated in vacuo resulting in the crude compound. The crude compound was purified through 100-200 mesh size silica gel column chromatography (elution: 0-50% ethyl acetate in n-hexane) to afford 3 (550 mg, 87.7%) as brown solid. ¹H NMR (DMSO-d₆, 400 MHz): δ ppm 12.84 (br. s, 1H), 8.04 (br. s, 1H), 7.68 (d, J=8.80 Hz, 1H), 6.88 (s, 1H), 6.69 (d, J=8.80 Hz, 1H), 3.00 (d, J=6.85 Hz, 2H), 1.80-1.92 (m, 1H), 0.95 (d, J=6.85 Hz, 6H); LC-MS: m/z 272.05 [M+H]⁺.

### Step-2: 7-Bromo-1-Isobutyl-2H-Benzo[d][1,3]Oxazine-2,4(1H)-Dione (5)

To a stirred mixture of 4-bromo-2-(isobutylamino)benzoic acid (3) (540 mg, 1.984 mmol, 1 eq) in ethyl acetate (20 mL) at 0° C. was added triphosgene (4) (294 mg, 0.992 mmol, 0.5 eq) and potassium carbonate (410 mg, 2.976 mmol, 1.5 eq) under nitrogen atmosphere. The reaction mixture was allowed to attain room temperature and stirred for 2 h. The reaction was monitored by TLC (M.Ph: 20% ethyl acetate in n-hexane). The reaction mixture was diluted with ethyl acetate (200 mL) and washed with water (2 × 50 mL) followed by brine (20 mL). The organic layer was separated, dried over anhydrous sodium sulfate, filtered and concentrated in vacuo resulting in the crude compound. The crude compound was purified by trituration, filtered and dried under vacuum to afford 5 (550 g, 93%) as an off white solid. ¹H NMR (DMSO-d₆, 400 MHz): δ ppm 7.91 (d, J=8.31 Hz, 1H), 7.74 (s, 1H), 7.51 (d, J=8.31 Hz, 1H), 3.88 (d, J=7.34 Hz, 2H), 2.07 (td, J=6.85, 13.69 Hz, 1H), 0.94 (d, J=6.36 Hz, 6H); LC-MS: m/z 299.95 [M+H]⁺.

### Step-3: Ethyl 7-Bromo-4-Hydroxy-1-Isobutyl-2-Oxo-1,2-Dihydroquinoline-3-Carboxylate (7)

To a stirred solution of 7-bromo-1-isobutyl-2H-benzo[d][1,3]oxazine-2,4(1H)-dione (5) (545 mg, 1.828 mmol, 1 eq) and diethyl malonate (6) (380 mg, 2.376 mmol, 1.3 eq) in DMA (50 mL) at 0° C. was added sodium hydride (60% dispersion in oil, 109 mg, 2.742 mmol, 1.5 eq) under nitrogen atmosphere and stirred for 15 min. The reaction mixture was further heated at 110° C. for 2 h. The reaction was monitored by TLC (M.Ph: 50% ethyl acetate in n-hexane). The reaction mixture was cooled to room temperature and poured into ice cold water (50 mL). The aqueous layer was separated and extracted with EtOAc (2 × 100 mL). The combined organic layer was washed with water (3 × 20 mL) followed by brine (10 mL). The organic layer was separated, dried over anhydrous sodium sulfate, filtered and concentrated in vacuo resulting in the crude compound. The crude compound was purified by 100-200 mesh size silica gel column chromatography (elution: 20-80% ethyl acetate in n-hexane) to afford 7 (440 mg, 65.3%) as an off white solid. ¹H NMR (DMSO-d₆, 400 MHz): δ ppm 12.96 (br. s, 1H), 7.96 (d, J=8.31 Hz, 1H), 7.75 (s, 1H), 7.46 (dd, J=1.22, 8.56 Hz, 1H), 4.31 (q, J=7.34 Hz, 2H), 4.07 (d, J=7.34 Hz, 2H), 2.00-2.11 (m, 1H), 1.30 (t, J=7.09 Hz, 3H), 0.88 (d, J=6.85 Hz, 6H); LC-MS: m/z 367.85 [M+H]⁺.

### Step-4: 7-Bromo-4-Hydroxy-1-Isobutyl-N-(3-Methylpyridin-2-yl)-2-Oxo-1,2-Dihydroquinoline-3-Carboxamide (SSTN-564)

To a stirred mixture of ethyl 7-bromo-4-hydroxy-1-isobutyl-2-oxo-1,2-dihydroquinoline-3-carboxylate (7) (200 mg, 0.543 mmol, 1 eq) in DMSO (10 mL) was added 3-methylpyridin-2-amine (8) (70.4 mg, 0.651 mmol, 1.2 eq) at room temperature. The reaction mixture was heated at 100° C. for 8 h. The progress of the reaction was monitored by TLC (M.Ph: 5% Methanol in DCM). The reaction mixture was cooled to room temperature and poured into ice cold water (50 mL). The aqueous layer was separated and extracted with EtOAc (2 x 100 mL). The combined organic layer was washed with water (3 × 20 mL) followed by brine (10 mL). The organic layer was separated, dried over anhydrous sodium sulfate, filtered and concentrated in vacuo resulting in the crude compound. The crude compound was purified through 100-200 mesh size silica gel column chromatography (elution: 2-8% methanol in DCM) to afford SSTN-564 (80 mg, 34.3%) as an off white solid. ¹H NMR (DMSO-d₆, 400 MHz): δ ppm 16.71 (br. s, 1H), 12.34 (br. s, 1H), 8.32 (d, J=3.42 Hz, 1H), 8.06 (d, J=8.31 Hz, 1H), 7.95 (br. s, 1H), 7.78 (d, J=6.36 Hz, 1H), 7.59 (d, J=7.82 Hz, 1H), 7.25-7.34 (m, 1H), 4.23 (d, J=6.36 Hz, 2H), 2.29 (s, 3H), 2.11-2.19 (m, 1H), 0.94 (d, J=6.36 Hz, 6H); LC-MS: m/z 429.91 [M+H]⁺; HPLC: 97.48%.

SSTN-565

Synthesis of 5-bromo-N-(3-fluorophenyl)-4-hydroxy-1-isobutyl-2-oxo-1,2-dihydroquinoline-3-carboxamide (SSTN-565)

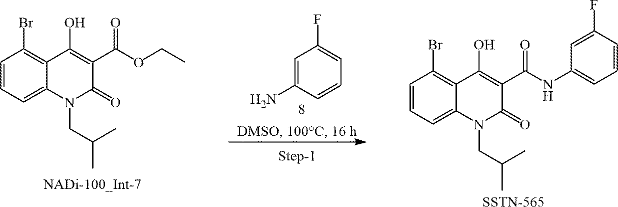

### Step-1: 5-Bromo-N-(3-Fluorophenyl)-4-Hydroxy-1-Isobutyl-2-Oxo-1,2-Dihydroquinoline-3-Carboxamide (SSTN-565)

To a stirred mixture of ethyl 5-bromo-4-hydroxy-1-isobutyl-2-oxo-1,2-dihydroquinoline-3-carboxylate (500 mg, 1.357 mmol, 1 eq) in DMSO (5 mL) was added 3-fluoroaniline (8) (226 mg, 2.036 mmol, 1.2 eq) at room temperature. The reaction mixture was heated at 100° C. for 16 h. The progress of the reaction was monitored by TLC (M.Ph: 30% ethyl acetate in n-hexane). The reaction mixture was poured into ice cold water (50 mL) and extracted with ethyl acetate (100 mL). The organic layer was washed with water (2 x 100 mL) followed by brine (2 × 100 mL). The organic layer was separated, dried over anhydrous sodium sulfate, filtered and concentrated in vacuo resulting in the crude compound. The crude compound was purified through 100-200 mesh size silica gel column chromatography (elution: 0-10% ethyl acetate in n-hexane) to afford SSTN-565 (100 mg, 17%) as an off white solid. ¹H NMR (DMSO-d₆, 400 MHz): δ ppm 17.47 (s, 1H), 12.94 (br. s, 1H), 7.77 (d, J=8.31 Hz, 1H), 7.60-7.72 (m, 3H), 7.37-7.51 (m, 2H), 7.06 (t, J=7.34 Hz, 1H), 4.24 (br. s, 2H), 2.15 (td, J=6.54, 12.84 Hz, 1H), 0.93 (d, J=6.36 Hz, 6H); LC-MS: m/z 432.80 [M+H]⁺; HPLC: 96.72%.

SSTN-566

Synthesis of 6-bromo-N-(3-fluorophenyl)-4-hydroxy-1-isobutyl-2-oxo-1,2-dihydroquinoline-3-carboxamide (SSTN-566)

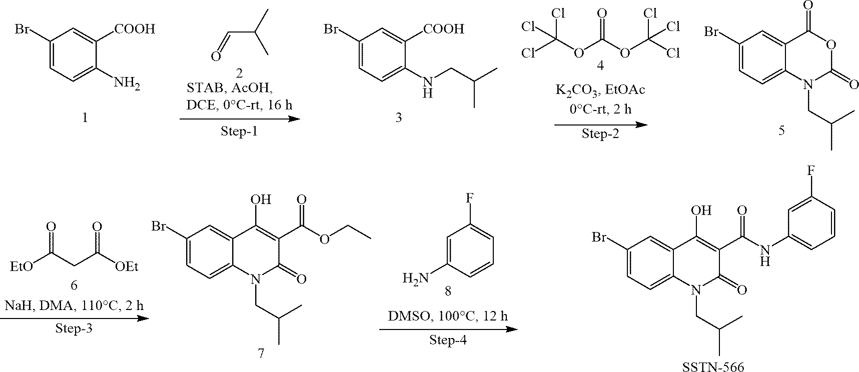

### Step-1: 5-Bromo-2-(Isobutylamino)Benzoic Acid (3)

To a stirred solution of 2-amino-5-bromobenzoic acid (1) (10.0 g, 46.28 mmol, 1 eq) in DCE (400 mL) was added isobutyraldehyde (2) (3.67 g, 50.91 mmol, 1.1 eq) at room temperature. To the resulting solution at 0° C. was added STAB (39.2 g, 185.15 mmol, 4 eq) lotwise followed by acetic acid (13.2 mL, 231.44 mmol, 5 eq) and stirred for 5 min. The reaction mixture was allowed to attain room temperature and stirred for 16 h. The reaction was monitored by TLC (M.Ph: 5% methanol in DCM). The reaction mixture was concentrated in vacuo upto dryness. The crude residue obtained was dissolved in DCM (500 mL) and washed with water (500 mL) and extracted with ethyl acetate (2 × 500 mL). The combined organic layer was dried over anhydrous sodium sulfate, filtered and concentrated in vacuo resulting in the crude compound. The crude compound was purified through 230-400 mesh size silica gel column chromatography (elution: 0-1% methanol in DCM) to afford 3 (10 g, 79.7%) as white solid. ¹H NMR (DMSO-d₆, 400 MHz): δ ppm 7.79 (br. s, 1H), 7.42 (d, J=8.80 Hz, 1H), 6.68 (d, J=8.80 Hz, 1H), 3.06-3.18 (m, 1H), 2.93 (d, J=5.87 Hz, 2H), 1.80 (td, J=6.30, 12.35 Hz, 1H), 0.88 (d, J=5.87 Hz, 6H); LC-MS: m/z 271.80 [M+H]⁺.

### Step-2: 6-Bromo-1-Isobutyl-2H-Benzo[d][1,3]Oxazine-2,4(1H)-Dione (5)

To a stirred mixture of 5-bromo-2-(isobutylamino)benzoic acid (3) (2.00 g, 7.379 mmol, 1 eq) in ethyl acetate (150 mL) at 0° C. was added triphosgene (4) (1.09 g, 3.689 mmol, 0.5 eq) and potassium carbonate (1.52 g, 11.06 mmol, 1.5 eq) and stirred for 5 min. The reaction mixture was allowed to attain room temperature and stirred for 2 h. The reaction was monitored by TLC (M.Ph: 20% ethyl acetate in n-hexane). The reaction mixture was poured over ice cold water (100 mL) and extracted with ethyl acetate (2 × 100 mL). The combined organic layer was washed with water (2 × 200 mL) followed by brine (200 mL). The organic layer was separated, dried over anhydrous sodium sulfate, filtered and concentrated in vacuo resulting in the crude compound. The crude compound was purified by 100-200 mesh size silica gel column chromatography (elution: 0-15% ethyl acetate in n-hexane) to afford 5 (1.65 g, 75.3%) as white solid. ¹H NMR (DMSO-d₆, 400 MHz): δ ppm 8.07 (d, J=2.45 Hz, 1H), 7.96 (dd, J=2.20, 9.05 Hz, 1H), 7.47 (d, J=8.80 Hz, 1H), 3.85 (d, J=7.34 Hz, 2H), 2.02-2.13 (m, 1H), 0.94 (d, J=6.85 Hz, 6H).

### Step-3: Ethyl 6-Bromo-4-Hydroxy-1-Isobutyl-2-Oxo-1,2-Dihydroquinoline-3-Carboxylate (7)

To a stirred solution of 6-bromo-1-isobutyl-2H-benzo[d][1,3]oxazine-2,4(1H)-dione (5) (1.60 g, 5.351 mmol, 1 eqv) and diethyl malonate (6) (1.11 g, 6.956 mmol, 1.3 eq) in DMA (50 mL) at 0° C. was added sodium hydride (60% dispersion in oil, 321 mg, 8.026 mmol, 1.5 eq) under nitrogen atmosphere. The reaction mixture was further heated at 110° C. for 2 h. The reaction was monitored by TLC (M.Ph: 30% ethyl acetate in n-hexane). The reaction mixture was poured over ice cold water and acidified with 1N HCl solution and extracted with ethyl acetate (2 × 250 mL). The combined organic layer was washed with water (2 × 250 mL) followed by brine (2 × 250 mL). The organic layer was separated, dried over anhydrous sodium sulfate, filtered and concentrated in vacuo resulting in the crude compound. The crude compound was purified by 230-400 mesh size silica gel column chromatography (elution: 0-30% ethyl acetate in n-hexane) to afford 7 (1.10 g, 57.8%) as yellow solid. LC-MS: m/z 367.80 [M+H]⁺.

### Step-4: 6-Bromo-N-(3-Fluorophenyl)-4-Hydroxy-1-Isobutyl-2-Oxo-1,2-Dihydroquinoline-3-Carboxamide (SSTN-566)

To a stirred mixture of ethyl 6-bromo-4-hydroxy-1-isobutyl-2-oxo-1,2-dihydroquinoline-3-carboxylate (7) (1.00 g, 2.715 mmol, 1 eq) in DMSO (10 mL) was added 3-fluoroaniline (8) (450 mg, 4.072 mmol, 1.5 eq) at room temperature. The reaction mixture was further heated at 100° C. for 12 h. The progress of the reaction was monitored by TLC (M.Ph: 70% ethyl acetate in n-hexane). The reaction mixture was cooled to room temperature and poured over ice cold water (100 mL) and extracted with ethyl acetate (2 × 100 mL). The combined organic layer was washed with water (2 × 100 mL) followed by brine (2 × 100 mL). The organic layer was separated, dried over anhydrous sodium sulfate, filtered and concentrated in vacuo resulting in the crude compound. The crude compound was purified through 100-200 mesh size silica gel column chromatography (elution: 10-90% ethyl acetate in n-hexane) to afford SSTN-566 (400 mg, 34%) as an off white solid. ¹H NMR (DMSO-d₆, 400 MHz): δ ppm 16.40 (br. s, 1H), 12.71 (br. s, 1H), 8.21 (br. s, 1H), 7.96 (d, J=8.80 Hz, 1H), 7.66-7.76 (m, 2H), 7.40-7.50 (m, 2H), 7.05 (t, J=7.58 Hz, 1H), 4.20 (d, J=5.87 Hz, 2H), 2.10-2.20 (m, 1H), 0.93 (d, J=6.36 Hz, 6H); LC-MS: m/z 432.90 [M+H]⁺; HPLC: 98.45%

SSTN-567

Synthesis of 7-bromo-N-(3-fluorophenyl)-4-hydroxy-1-isobutyl-2-oxo-1,2-dihydroquinoline-3-carboxamide (SSTN-567)

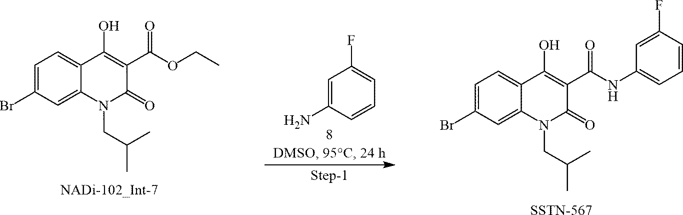

### Step-1: 7-Bromo-N-(3-Fluorophenyl)-4-Hydroxy-1-Isobutyl-2-Oxo-1,2-Dihydroquinoline-3-Carboxamide (SSTN-567)

To a stirred solution of ethyl 7-bromo-4-hydroxy-1-isobutyl-2-oxo-1,2-dihydroquinoline-3-carboxylate (200 mg, 0.543 mmol, 1 eq) in DMSO (10 mL) was added 3-fluoroaniline (8) (72.4 mg, 0.651 mmol, 1.2 eq) at room temperature. The reaction mixture was heated at 95° C. for 24 h. The progress of the reaction was monitored by TLC (M.Ph: 30% ethyl acetate in n-hexane). The reaction mixture was diluted with water (50 mL) and extracted with ethyl acetate (2 × 50 mL). The combined organic layer was washed with water (20 mL) followed by brine (20 mL). The organic layer was separated, dried over anhydrous sodium sulfate, filtered and concentrated in vacuo resulting in the crude compound. The crude compound was purified through 100-200 mesh size silica gel column chromatography (elution: 0-30% ethyl acetate in n-hexane) to afford SSTN-567 (115 mg, 48.9%) as an off white solid. ¹H NMR (DMSO-d₆, 400 MHz): δ ppm 16.40 (br. s, 1H), 12.68 (br. s, 1H), 8.07 (d, J=8.80 Hz, 1H), 7.92-7.99 (m, 1H), 7.71 (d, J=11.25 Hz, 1H), 7.59 (br. s, 1H), 7.38-7.49 (m, 3H), 7.03 (br. s, 1H), 4.21 (d, J=3.42 Hz, 2H), 2.15 (dt, J=6.36, 13.20 Hz, 2H), 0.93 (d, J=6.36 Hz, 6H); LC-MS: m/z 433.00 [M+H]⁺; HPLC: 98.36%

SSTN-568 (free base, HCl salt)

Synthesis of 4-hydroxy-1-isobutyl-N-(2-(4-methylpiperazin-1-yl)phenyl)-2-oxo-1,2-dihydroquinoline-3-carboxamide (SSTN-568, Notch1 Reporter Assay IC₅₀: 21.5 µM)

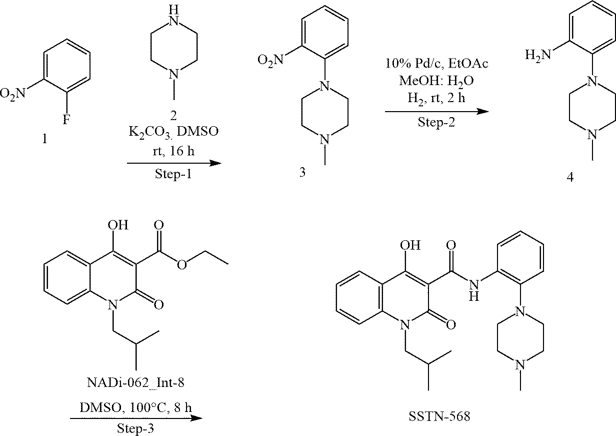

### Step-1: 1-Methyl-4-(2-Nitrophenyl)Piperazine (3)

To a stirred mixture of 1-methylpiperazine (2) (1.70 g, 17.00 mmol, 1.2 eq) in DMSO (15 mL) was added potassium carbonate (3.89 g, 3.61 mmol, 1.2 eq) followed by 1-fluoro-2-nitrobenzene (1) (2.00 g, 14.17 mmol, 1 eq) at room temperature and stirred for 16 h. The reaction was monitored by TLC (M.Ph: 50% ethyl acetate in n-hexane). The reaction mixture was poured into water (100 mL) and extracted with ethyl acetate (2 × 200 mL). The combined organic layer was washed with water (3 × 50 mL) followed by brine (20 mL). The organic layer was dried over anhydrous sodium sulfate, filtered and concentrated in vacuo resulting in the crude compound. The crude compound was purified through 230-400 mesh size silica gel column chromatography (elution: 40-60% ethyl acetate in n-hexane) to afford 3 (2.83 g, 90.4%) as dark brown oil. ¹H NMR (DMSO-d₆, 400 MHz): δ ppm 7.85 (d, J=8.31 Hz, 1H), 7.64 (t, J=7.83 Hz, 1H), 7.38 (d, J=8.31 Hz, 1H), 7.18 (t, J=7.58 Hz, 1H), 3.00-3.10 (m, 4H), 2.46-2.53 (m, 4H), 2.28 (s, 3H); LC-MS: m/z 221.80 [M+H]⁺.

### Step-2: 2-(4-Methylpiperazin-1-yl)Aniline (4)

To a stirred solution of 1-methyl-4-(2-nitrophenyl)piperazine (3) (500 mg, 2.259 mmol, 1 eq) in mixture of MeOH: EtOAc: H₂O (3: 5: 1 mL) was added 10% Pd/C (100 mg) in (5 mL) under inert atmosphere at room temperature into a hydrogenator. The mixture was degassed for 15 min with the help of alternative vacuum and nitrogen. The reaction was hydrogenated for 2 h at 3.5 bar pressure. The reaction was monitored by TLC (M.Ph: 5% Methanol in DCM). The reaction mixture was filtered through Celite bed and the filtrate was concentrated in vacuo resulting in the crude compound. The crude compound was purified by trituration with n-hexane, filtered and dried in vacuum to afford 4 (350 mg, 81%) as yellow solid. ¹H NMR (DMSO-d₆, 400 MHz): δ ppm 6.88 (d, J=7.82 Hz, 1H), 6.77-6.82 (m, 1H), 6.66 (d, J=7.83 Hz, 1H), 6.53 (t, J=7.58 Hz, 1H), 4.66 (br. s, 2H), 2.74-2.81 (m, 4H), 2.41-2.49 (m, 4H), 2.22 (s, 3H); LC-MS: m/z 191.80 [M+H]⁺.

### Step-3: 4-Hydroxy-1-Isobutyl-N-(2-(4-Methylpiperazin-1-yl)Phenyl)-2-Oxo-1,2-Dihydroquinoline-3-Carboxamide (SSTN-568)

To a stirred mixture of ethyl 4-hydroxy-1-isobutyl-2-oxo-1,2-dihydroquinoline-3-carboxylate (453 mg, 1.565 mmol, 1.2 eq) in DMSO (10 mL) was added 2-(4-methylpiperazin-1-yl)aniline (4) (250 mg, 1.306 mmol, 1 eq) at room temperature. The reaction mixture was heated at 100° C. for 8 h. The progress of the reaction was monitored by TLC (M.Ph: 5% Methanol in DCM). The reaction mixture was cooled to room temperature, poured into ice cold water (50 mL). The aqueous layer was separated and extracted with ethyl acetate (2 × 100 mL). The combined organic layer was washed with ice cold water (2 x 30 mL) followed by brine (10 mL). The organic layer was separated, dried over anhydrous sodium sulfate, filtered and concentrated in vacuo resulting in the crude compound. The crude compound was purified through 230-400 mesh size silica gel column chromatography (elution: 0-5% methanol in DCM) to afford SSTN-568 (150 mg, 26.4%) as an off white solid. ¹H NMR (DMSO-d₆, 400 MHz): δ ppm 16.88 (br. s, 1H), 13.02 (br. s, 1H), 8.29-8.34 (m, 1H), 8.16 (d, J=7.83 Hz, 1H), 7.79-7.86 (m, 1H), 7.70 (d, J=8.80 Hz, 1H), 7.39 (t, J=7.58 Hz, 1H), 7.29 (dd, J=3.42, 5.87 Hz, 1H), 7.14-7.19 (m, 2H), 4.23 (d, J=7.34 Hz, 2H), 2.86 (t, J=4.40 Hz, 4H), 2.60-2.68 (m, 4H), 2.27 (s, 3H), 2.16-2.24 (m, 1H), 0.98 (d, J=6.85 Hz, 6H); LC-MS: m/z 435.00 [M+H]⁺; HPLC: 99.75%

Synthesis of 4-hydroxy-1-isobutyl-N-(2-(4-methylpiperazin-1-yl)phenyl)-2-oxo-1,2-dihydroquinoline-3-carboxamide hydrochloride salt (SSTN-568_HCl salt, Notch1 Reporter Assay IC₅₀: 6.83 µM)

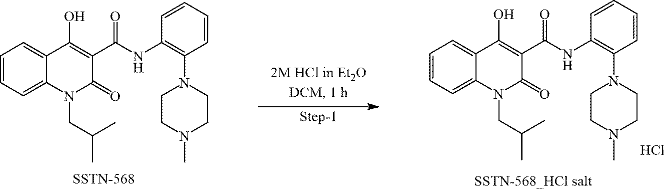

### Step-1: Synthesis of 4-Hydroxy-1-Isobutyl-N-(2-(4-Methylpiperazin-1-yl)Phenyl)-2-Oxo-1,2-Dihydroquinoline-3-Carboxamide Hydrochloride Salt (SSTN-568_HCl Salt)

To a stirred mixture of 4-hydroxy-1-isobutyl-N-(2-(4-methylpiperazin-1-yl)phenyl)-2-oxo-1,2-dihydroquinoline-3-carboxamide (SSTN-568) (20 mg, 0.046 mmol, 1 eq) in Et₂O (1 mL) at was added 2 M HCl in dioxane (100 µL). The reaction mixture was stirred for 1 h then concentrated in vacuo resulting in the crude compound. The crude compound was purified by trituration with Et₂O/DCM, filtered and dried under vacuum to afford SSTN-568-_HCl salt (19.5 mg, 90%) an off-white solid. ¹H NMR (400 MHz, DMSO) δ 10.52 (s, 1H), 8.38 (dd, J=7.7, 1.9 Hz, 1H), 8.17 (dd, J=8.1, 1.6 Hz, 1H), 7.84 (ddd, J=8.7, 7.0, 1.7 Hz, 1H), 7.72 (d, J= 8.7 Hz, 1H), 7.41 (t, J= 7.6 Hz, 1H), 7.32 (dd, J= 7.4, 1.9 Hz, 1H), 7.28 -7.15 (m, 2H), 4.26 (d, J= 7.3 Hz, 2H), 3.58 (d, J= 11.4 Hz, 2H), 3.32 (s, 2H), 3.22 (d, J= 12.9 Hz, 2H), 3.13 (d, J= 11.5 Hz, 4H), 2.89 (s, 3H), 2.18 (dt, J= 13.7, 6.9 Hz, 1H), 0.98 (d, J= 6.6 Hz, 5H); LC-MS: m/z 435.00 [M+H]⁺.

SSTN-569 (free base, HCl salt)

Synthesis of 4-hydroxy-1-isobutyl-N-(4-(4-methylpiperazin-1-yl)phenyl)-2-oxo-1,2-dihydroquinoline-3-carboxamide (SSTN-569, Notch1 Reporter Assay IC₅₀: 8.42 µM)

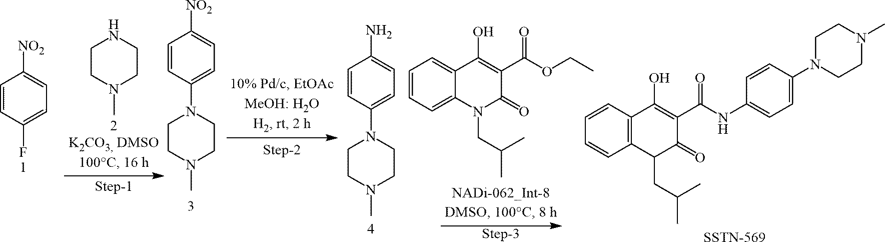

### Step-1: 1-Methyl-4-(4-Nitrophenyl)Piperazine (3)

To a stirred mixture of 1-fluoro-4-nitrobenzene (1) (2.00 g, 14.18 mmol, 1 eq) and 1-methylpiperazine (2) (1.56 g, 15.60 mmol, 1.2 eq) in DMSO (20 mL) was added potassium carbonate (5.87 g, 42.55 mmol, 3 eq) at room temperature. The reaction mixture was further heated at 100° C. for 16 h. The reaction was monitored by TLC (M.Ph: 5% methanol in DCM). The reaction mixture was poured into ice cold water (300 mL) and extracted with ethyl acetate (2 × 250 mL). The combined organic layer was washed with water (2 × 300 mL). The organic layer was dried over anhydrous sodium sulfate, filtered and concentrated in vacuo resulting in the crude compound. The crude compound was purified through 230-400 mesh size silica gel column chromatography (elution: 0-5% methanol in DCM) to afford 3 (3.00 g, 95.6%) as yellow solid. ¹H NMR (DMSO-d₆, 400 MHz): δ ppm 8.09-8.15 (m, 2H), 7.07-7.13 (m, 2H), 3.48-3.57 (m, 4H), 2.47-2.54 (m, 4H), 2.30 (s, 3H); LC-MS: m/z 221.90 [M+H]⁺.

### Step-2: 4-(4-Methylpiperazin-1-yl)Aniline (4)

To a stirred solution of 1-methyl-4-(4-nitrophenyl)piperazine (3) (1 g, 4.519 mmol, 1 eq) in mixture of MeOH: EtOAc: H₂O (5: 4: 2 mL) was added 10% Pd/C (300 mg) under inert atmosphere at room temperature into a hydrogenator. The mixture was degassed for 15 min with the help of alternative vacuum and nitrogen. The reaction was stirred under hydrogen atmosphere for 2 h at room temperature. The reaction was monitored by TLC (M.Ph: 5% methanol in DCM). The reaction mixture was filtered through Celite bed and the filtrate was concentrated in vacuo to afford 4 (750 mg, 86.8%) as yellow solid. ¹H NMR (DMSO-d₆, 400 MHz): δ ppm 6.75 (d, J=8.80 Hz, 2H), 6.56 (d, J=8.80 Hz, 2H), 4.63 (br. s, 2H), 2.93-3.02 (m, 4H), 2.46-2.54 (m, 4H), 2.28 (s, 3H); LC-MS: m/z 191.80 [M+H]⁺.

### Step-3: 4-Hydroxy-1-Isobutyl-N-(4-(4-Methylpiperazin-1-yl)Phenyl)-2-Oxo-1,2-Dihydroquinoline-3-Carboxamide (SSTN-569)

To a stirred mixture of ethyl 4-hydroxy-1-isobutyl-2-oxo-1,2-dihydroquinoline-3-carboxylate (453 mg, 1.565 mmol, 1.2 eq) in DMSO (5 mL) was added 4-(4-methylpiperazin-1-yl)aniline (4) (250 mg, 1.306 mmol, 1 eq) at room temperature. The reaction mixture was heated at 100° C. for 8 h. The progress of the reaction was monitored by TLC (M.Ph: 5% Methanol in DCM). The reaction mixture was poured into ice cold water (200 mL) and extracted with ethyl acetate (2 × 250 mL). The combined organic layer was washed with ice cold water (2 × 200 mL). The organic layer was separated, dried over anhydrous sodium sulfate, filtered and concentrated in vacuo resulting in the crude compound. The crude compound was purified through 230-400 mesh size silica gel column chromatography (elution: 0-5% methanol in DCM) to afford SSTN-569 (128 mg, 22.5%) as a yellow solid. ¹H NMR (DMSO-d₆, 400 MHz): δ ppm 16.94 (br. s, 1H), 12.50 (br. s, 1H), 8.15 (d, J=7.82 Hz, 1H), 7.78-7.86 (m, 1H), 7.72 (d, J=8.80 Hz, 1H), 7.53 (d, J=9.29 Hz, 2H), 7.41 (t, J=7.34 Hz, 1H), 6.97 (d, J=8.80 Hz, 2H), 4.22 (d, J=5.87 Hz, 2H), 3.10-3.17 (m, 4H), 2.44-2.48 (m, 4H), 2.23 (s, 3H), 2.14-2.21 (m, 1H), 0.93 (d, J=6.36 Hz, 6H). LC-MS: m/z 435.00 [M+H]⁺; HPLC: 95.02%.

Synthesis of 4-hydroxy-1-isobutyl-N-(4-(4-methylpiperazin-1-yl)phenyl)-2-oxo-1,2-dihydroquinoline-3-carboxamide hydrochloride salt (SSTN-569_HCl salt, Notch1 Reporter Assay IC₅₀: 4.93 µM)

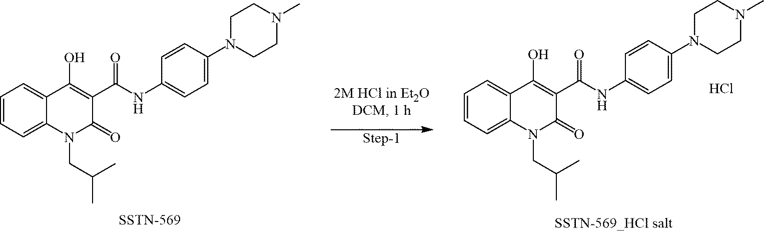

### Step-1: Synthesis of 4-Hydroxy-1-Isobutyl-N-(4-(4-Methylpiperazin-1-yl)Phenyl)-2-Oxo-1,2-Dihydroquinoline-3-Carboxamide Hydrochloride Salt (SSTN-569_HCl Salt)

To a stirred mixture of 4-hydroxy-1-isobutyl-N-(4-(4-methylpiperazin-1-yl)phenyl)-2-oxo-1,2-dihydroquinoline-3-carboxamide (SSTN-569) (20 mg, 0.046 mmol, 1 eq) in Et₂O (1 mL) at was added 2 M HCl in dioxane (100 µL). The reaction mixture was stirred for 1 h then concentrated in vacuo resulting in the crude compound. The crude compound was purified by trituration with Et₂O/DCM, filtered and dried under vacuum to afford SSTN-569_HCl salt (18.4 mg, 84%) an off-white solid. ¹H NMR (400 MHz, DMSO) δ 10.44 (s, 1H), 8.14 (dd, J= 8.0, 1.6 Hz, 1H), 7.82 (ddd, J= 8.7, 7.0, 1.6 Hz, 1H), 7.72 (d, J= 8.7 Hz, 1H), 7.62 - 7.54 (m, 2H), 7.40 (t, J= 7.5 Hz, 1H), 7.09 - 7.01 (m, 2H), 4.21 (d, J= 7.5 Hz, 2H), 3.82 (d, J= 12.3 Hz, 2H), 3.48 (s, 2H), 3.32 (s, 1H), 3.23 - 2.95 (m, 4H), 2.82 (s, 3H), 2.17 (dt, J= 13.8, 6.9 Hz, 1H), 0.92 (d, J= 6.7 Hz, 6H); LC-MS: m/z 435.0 [M+H]⁺.

SSTN-570 (free base, HCl salt)

Synthesis ofN-(4-fluoro-2-(4-methylpiperazin-1-yl)phenyl)-4-hydroxy-1-isobutyl-2-oxo-1,2-dihydroquinoline-3-carboxamide (SSTN-570, Notch1 Reporter Assay IC₅₀: 6.5 µM)

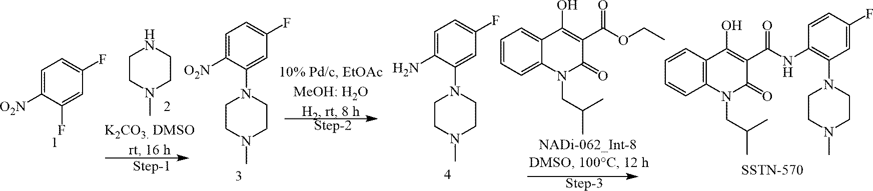

### Step-1: 1-(5-Fluoro-2-Nitrophenyl)-4-Methylpiperazine (3)

To a stirred mixture of 2,4-difluoro-1-nitrobenzene (1) (1.00 g, 6.285 mmol, 1 eq) and 1-methylpiperazine (2) (755 mg, 7.542 mmol, 1.2 eq) in DMSO (10 mL) was added potassium carbonate (1.73 g, 12.57 mmol, 3 eq) at room temperature and stirred for 16 h. The reaction was monitored by TLC (M.Ph: 30% ethyl acetate in n-hexane. The reaction mixture was diluted with ethyl acetate (250 mL) and washed with water (250 mL). The organic layer was separated, dried over anhydrous sodium sulfate, filtered and concentrated in vacuo to afford 3 (1.40 g, 93%) as yellow solid. Crude compound was used as such in the next step without further purification. ¹H NMR (DMSO-d₆, 400 MHz): δ ppm 7.83-8.01 (m, 1H), 7.06-7.14 (m, 1H), 6.82-6.92 (m, 1H), 2.95-3.06 (m, 4H), 2.37-2.46 (m, 4H), 2.21 (s, 3H); LC-MS: m/z 239.90 [M+H]⁺.

### Step-2: 4-Fluoro-2-(4-Methylpiperazin-1-yl)Aniline (4)

To a stirred solution of 1-(5-fluoro-2-nitrophenyl)-4-methylpiperazine (3) (1.00 g, 4.179 mmol, 1 eq) in mixture of MeOH: EtOAc: H₂O (5: 10: 0.5 mL) was added 10% Pd/C (300 mg) under inert atmosphere at room temperature into a hydrogenator. The mixture was degassed for 15 min with the help of alternative vacuum and nitrogen. The reaction was stirred under hydrogen atmosphere for 8 h at room temperature. The reaction was monitored by TLC (M.Ph: 5% methanol in DCM). The reaction mixture was filtered through Celite bed and the filtrate was concentrated in vacuo to afford 4 (800 mg, 91.5%) as yellow solid. LC-MS: m/z 209.90 [M+H]⁺.

### Step-3: N-(4-Fluoro-2-(4-Methylpiperazin-1-yl)Phenyl)-4-Hydroxy-1-Isobutyl-2-Oxo-1,2-Dihydroquinoline-3-Carboxamide (SSTN-570)

To a stirred mixture of ethyl 4-hydroxy-1-isobutyl-2-oxo-1,2-dihydroquinoline-3-carboxylate (300 mg, 1.036 mmol, 1 eq) in DMSO (5 mL) was added 4-fluoro-2-(4-methylpiperazin-1-yl)aniline (4) (260 mg, 1.244 mmol, 1.2 eq) at room temperature. The reaction mixture was further heated at 100° C. for 12 h in a sealed tube. The progress of the reaction was monitored by TLC (M.Ph: 5% Methanol in DCM). The reaction mixture was poured into ice cold water (50 mL) and extracted with ethyl acetate (2 × 50 mL). The combined organic layer was washed with ice cold water (2 × 50 mL) followed by brine (50 mL). The organic layer was separated, dried over anhydrous sodium sulfate, filtered and concentrated in vacuo resulting in the crude compound. The crude compound was purified preparative HPLC to afford SSTN-570 (10 mg, 2.13%) as an off white solid. ¹H NMR (CDC1₃, 400 MHz): δ ppm 16.66 (s, 1H), 12.53 (br. s, 1H), 8.26 (d, J=7.88 Hz, 1H), 8.09 (t, J=9.16 Hz, 1H), 7.68 (t, J=7.38 Hz, 1H), 7.37 (d, J=8.39 Hz, 1H), 7.30 (t, J=7.63 Hz, 1H), 6.68-6.76 (m, 2H), 4.16-4.25 (m, 2H), 3.19-3.26 (m, 4H), 2.54-2.63 (m, 4H), 2.36 (s, 3H), 2.26 (td, J=6.68, 13.61 Hz, 1H), 1.01 (d, J=6.61 Hz, 6H); LC-MS: m/z 453.10 [M+H]⁺; HPLC: 95.69%

Synthesis ofN-(4-fluoro-2-(4-methylpiperazin-1-yl)phenyl)-4-hydroxy-1-isobutyl-2-oxo-1,2-dihydroquinoline-3-carboxamide hydrochloride salt (SSTN-570_HCl salt, Notch1 Reporter Assay IC₅₀: 5.6 µM)

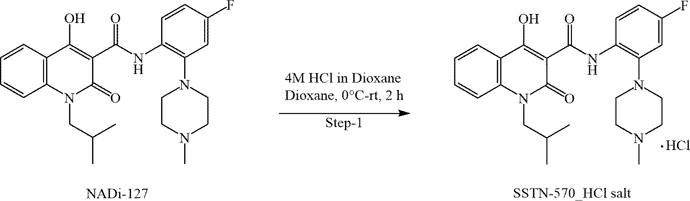

### Step-1: N-(4-Fluoro-2-(4-Methylpiperazin-1-yl)Phenyl)-4-Hydroxy-1-Isobutyl-2-Oxo-1,2-Dihydroquinoline-3-Carboxamide Hydrochloride Salt (SSTN-570_HCl Salt)

To a stirred mixture of N-(4-fluoro-2-(4-methylpiperazin-1-yl)phenyl)-4-hydroxy-1-isobutyl-2-oxo-1,2-dihydroquinoline-3-carboxamide (SSTN-570) (50 mg, 0.110 mmol, 1 eq) in dioxane (5 mL) at 0° C. was added 4 M HCl in dioxane (1 mL). The reaction mixture was allowed to attain at room temperature and stirred for 2 h. The progress of the reaction was monitored by TLC (M.Ph: 5% Methanol in DCM). The reaction mixture was concentrated in vacuo resulting in the crude compound. The crude compound was purified by trituration with n-hexane (5 mL), filtered and dried under vacuum to afford SSTN-570_HCl salt (44 mg, 81.4%) as an off white solid. ¹H NMR (DMSO-d₆, 400 MHz): δ ppm 16.69 (s, 1H), 12.95 (br. s, 1H), 10.51 (br. s, 1H), 8.37 (dd, J=6.36, 8.31 Hz, 1H), 8.17 (d, J=7.83 Hz, 1H), 7.85 (t, J=7.09 Hz, 1H), 7.73 (d, J=8.31 Hz, 1H), 7.42 (t, J=7.34 Hz, 1H), 7.23 (d, J=7.83 Hz, 1H), 7.09 (t, J=7.09 Hz, 1H), 4.27 (d, J=3.42 Hz, 2H), 3.40-3.65 (m, 5H), 3.12 (d, J=10.76 Hz, 3H), 2.89 (br. s, 3H), 2.13-2.23 (m, 1H), 0.98 (d, J=6.36 Hz, 6H); LC-MS: m/z 453.10 [M+H]⁺; HPLC: 97.66%.

SSTN-571 (free base, HCl salt)

Synthesis of N-(5-fluoro-2-(4-methylpiperazin-1-yl)phenyl)-4-hydroxy-1-isobutyl-2-oxo-1,2-dihydroquinoline-3-carboxamide (SSTN-571, Notch1 Reporter Assay IC₅₀: n/a)

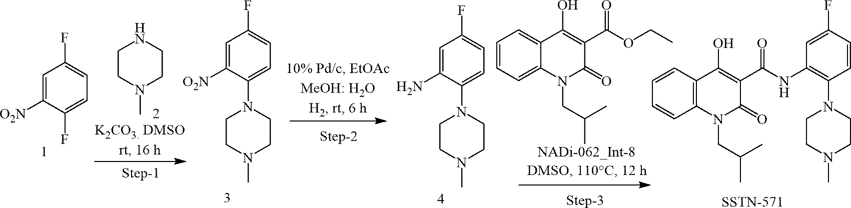

### Step-1: 1-(4-fluoro-2-nitrophenyl)-4-methylpiperazine (3)

To a stirred mixture of 1,4-difluoro-2-nitrobenzene (1) (1.00 g, 6.285 mmol, 1 eq) and 1-methylpiperazine (2) (755 mg, 7.542 mmol, 1.2 eq) in DMSO (10 mL) was added potassium carbonate (1.73 g, 12.57 mmol, 3 eq) at room temperature and stirred for 16 h. The reaction was monitored by TLC (M.Ph: 5% methanol in DCM). The reaction mixture was poured into ice cold water (150 mL) and extracted with ethyl acetate (250 mL). The organic layer was washed with ice cold water (2 × 200 mL) followed by brine (2 × 200 mL). The organic layer was separated, dried over anhydrous sodium sulfate, filtered and concentrated in vacuo to afford 3 (1.45 g, 96.6%) as yellow solid. Crude compound was used as such in the next step without further purification. ¹H NMR (DMSO-d₆, 400 MHz): δ ppm 7.77-7.82 (m, 1H), 7.46-7.54 (m, 1H), 7.40-7.46 (m, 1H), 2.94 (d, J=3.56 Hz, 4H), 2.37-2.44 (m, 4H), 2.20 (s, 3H); LC-MS: m/z 239.90 [M+H]⁺.

### Step-2: 5-Fluoro-2-(4-Methylpiperazin-1-yl)Aniline (4)

To a stirred solution of 1-(4-fluoro-2-nitrophenyl)-4-methylpiperazine (3) (700 mg, 2.926 mmol, 1 eq) in mixture of MeOH: EtOAc: H₂O (3: 5: 1 mL) was added 10% Pd/C (300 mg) under inert atmosphere at room temperature into a hydrogenator. The mixture was degassed for 15 min with the help of alternative vacuum and nitrogen. The reaction was stirred under hydrogen atmosphere for 6 h at room temperature. The reaction was monitored by TLC (M.Ph: 10% methanol in DCM). The reaction mixture was filtered through Celite bed and the filtrate was concentrated in vacuo resulting in the crude compound. The crude compound was purified by trituration with diethyl ether and n-hexane, filtered and dried under vacuum to afford 4 (800 mg, 91.5%) as yellow solid. ¹H NMR (DMSO-d₆, 400 MHz): δ ppm 6.87 (dd, J=6.10, 8.39 Hz, 1H), 6.43 (dd, J=2.92, 11.06 Hz, 1H), 6.27 (dt, J=2.80, 8.65 Hz, 1H), 5.02 (br. s, 2H), 2.69-2.77 (m, 4H), 2.34-2.49 (m, 4H), 2.21 (s, 3H); LC-MS: m/z 210.25 [M+H]⁺.

### Step-3: N-(5-Fluoro-2-(4-Methylpiperazin-1-yl)Phenyl)-4-Hydroxy-1-Isobutyl-2-Oxo-1,2-Dihydroquinoline-3-Carboxamide (SSTN-571)

To a stirred mixture of ethyl 4-hydroxy-1-isobutyl-2-oxo-1,2-dihydroquinoline-3-carboxylate (230 mg, 0.795 mmol, 1 eq) in DMSO (5 mL) was added 5-fluoro-2-(4-methylpiperazin-1-yl)aniline (4) (200 mg, 0.954 mmol, 1.2 eq) at room temperature. The reaction mixture was further heated at 110° C. for 12 h in a sealed tube. The progress of the reaction was monitored by TLC (M.Ph: 5% Methanol in DCM). The reaction mixture was poured into ice cold water (50 mL) and extracted with ethyl acetate (2 × 50 mL). The combined organic layer was washed with ice cold water (2 × 50 mL) followed by brine (50 mL). The organic layer was separated, dried over anhydrous sodium sulfate, filtered and concentrated in vacuo resulting in the crude compound. The crude compound was purified through 230-400 mesh size silica gel column chromatography (elution: 0-3% methanol in DCM) to afford SSTN-571 (90 mg, 25%) as an off white solid. ¹H NMR (CDC1₃, 400 MHz): δ ppm 16.66 (br. s, 1H), 13.12 (br. s, 1H), 8.23-8.33 (m, 2H), 7.68 (t, J=7.88 Hz, 1H), 7.36 (d, J=8.65 Hz, 1H), 7.30 (t, J=7.63 Hz, 1H), 7.15 (dd, J=5.59, 8.65 Hz, 1H), 6.80 (dt, J=2.80, 8.27 Hz, 1H), 4.20 (d, J=6.10 Hz, 2H), 2.95 (d, J=4.07 Hz, 4H), 2.74-2.82 (m, 4H), 2.43 (s, 3H), 2.21-2.31 (m, 1H), 1.05 (d, J=6.61 Hz, 6H); LC-MS: m/z 453.00 [M+H]⁺; HPLC: 96.83%.

Synthesis ofN-(5-fluoro-2-(4-methylpiperazin-1-yl)phenyl)-4-hydroxy-1-isobutyl-2-oxo-1,2-dihydroquinoline-3-carboxamide hydrochloride salt (SSTN-571_HCl salt, Notch1 Reporter Assay IC₅₀: 7.87 µM)

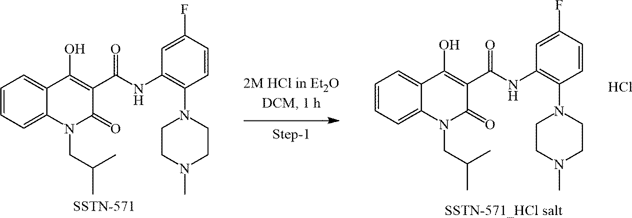

### Step-1: Synthesis of N-(5-Fluoro-2-(4-Methylpiperazin-1-yl)Phenyl)-4-Hydroxy-1-Isobutyl-2-Oxo-1,2-Dihydroquinoline-3-Carboxamide Hydrochloride Salt (SSTN-571_HCl Salt)

To a stirred mixture of N-(5-fluoro-2-(4-methylpiperazin-1-yl)phenyl)-4-hydroxy-1-isobutyl-2-oxo-1,2-dihydroquinoline-3-carboxamide (SSTN-571) (20 mg, 0.044 mmol, 1 eq) in Et₂O (1 mL) at was added 2 M HCl in dioxane (100 µL). The reaction mixture was stirred for 1 h then concentrated in vacuo resulting in the crude compound. The crude compound was purified by trituration with Et₂O/DCM, filtered and dried under vacuum to afford SSTN-571_HCl salt (15.1 mg, 70%) an off-white solid. ¹H NMR (400 MHz, DMSO) δ 10.39 (s, 1H), 8.22 (dd, J=11.2, 3.0 Hz, 1H), 8.17 (dd, J=8.0, 1.6 Hz, 1H), 7.85 (ddd, J=8.7, 7.0, 1.6 Hz, 1H), 7.73 (d, J= 8.7 Hz, 1H), 7.46 - 7.34 (m, 2H), 7.03 (td, J= 8.4, 3.0 Hz, 1H), 4.26 (d, J= 7.1 Hz, 2H), 3.64 - 3.55 (m, 2H), 3.42 - 3.31 (m, 3H), 3.19 (d, J= 12.7 Hz, 2H), 3.10 (t, J= 11.7 Hz, 2H), 2.90 (s, 3H), 2.17 (dq, J= 12.1, 6.3 Hz, 1H), 0.97 (d, J= 6.6 Hz, 6H); LC-MS: m/z 453.2 [M+H]⁺.

SSTN-572 (free base, HCl salt)

Synthesis of N-(3 -fluoro-4-(4-methylpiperazin-1 -yl)phenyl)-4-hydroxy-1 -isobutyl-2-oxo-1,2-dihydroquinoline-3-carboxamide (SSTN-572, Notch1 Reporter Assay IC₅₀: 17.7 µM)

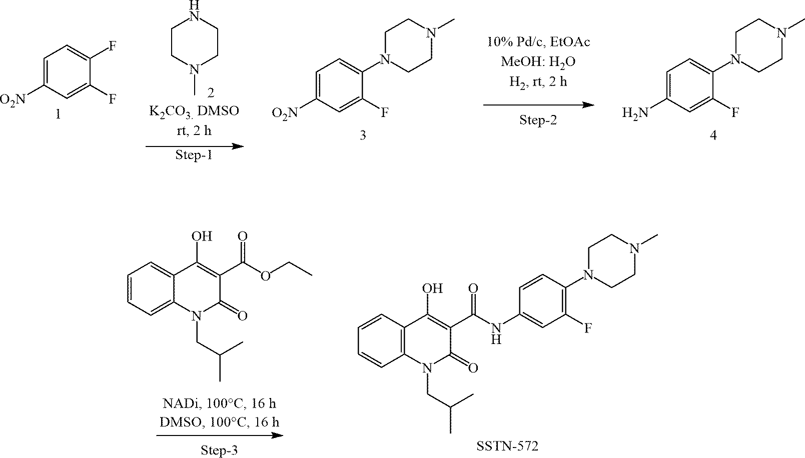

### Step-1: 1-(2-Fluoro-4-Nitrophenyl)-4-Methylpiperazine (3)

To a stirred mixture of 1,2-difluoro-4-nitrobenzene (1) (1.00 g, 6.289 mmol, 1 eq) and 1-methylpiperazine (2) (755 mg, 7.547 mmol, 1.2 eq) in DMSO (10 mL) at 0° C. was added potassium carbonate (1.73 g, 12.57 mmol, 2 eq). Reaction mixture was allowed to attain room temperature and stirred for 3 h. The reaction was monitored by TLC (M.Ph: 3% methanol in DCM). The reaction mixture was poured into ice cold water (100 mL) and extracted with ethyl acetate (2 × 50 mL). The combined organic layer was washed with water (2 × 100 mL) followed by brine (2 × 100 mL). The organic layer was separated, dried over anhydrous sodium sulfate, filtered and concentrated in vacuo to afford 3 (1.40 g, 93.3%) as brown solid. Crude compound obtained was used as such in the next step without further purification. ¹H NMR (DMSO-d₆, 400 MHz): δ ppm 8.04 (d, J=11.74 Hz, 2H), 7.20 (t, J=9.05 Hz, 1H), 3.30-3.35 (m, 4H), 2.47-2.52 (m, 4H), 2.26 (s, 3H); LC-MS: m/z 239.90 [M+H]⁺.

### Step-2: 3-Fluoro-4-(4-Methylpiperazin-1-yl)Aniline (4)

To a stirred solution of 1-(2-fluoro-4-nitrophenyl)-4-methylpiperazine (3) (700 mg, 2.926 mmol, 1 eq) in MeOH (5 mL) was added slurry of 10% Pd/C (250 mg) in ethyl acetate (5 mL) followed by water (1 mL) at room temperature into a hydrogenator. The mixture was degassed for 15 min with the help of alternative vacuum and nitrogen. The reaction was stirred under hydrogen atmosphere for 2 h at room temperature. The reaction was monitored by TLC (M.Ph: 5% methanol in DCM). The reaction mixture was filtered through Celite bed and the filtrate was concentrated in vacuo upto dryness to afford 4 (550 mg, 89.8%) as brown solid. Crude compound obtained was used as such in the next step without further purification. ¹H NMR (DMSO-d₆, 400 MHz): δ ppm 6.75 (t, J=9.29 Hz, 1H), 6.25-6.37 (m, 2H), 4.95 (br. s, 2H), 2.77-2.86 (m, 4H), 2.37-2.45 (m, 4H), 2.19 (s, 3H) ; LC-MS: m/z 210.10 [M+H]⁺.

### Step-3: N-(3-Fluoro-4-(4-Methylpiperazin-1-yl)Phenyl)-4-Hydroxy-1-Isobutyl-2-Oxo-1,2-Dihydroquinoline-3-Carboxamide(SSTN-572)

To a stirred mixture of ethyl 4-hydroxy-1-isobutyl-2-oxo-1,2-dihydroquinoline-3-carboxylate (300 mg, 1.036 mmol, 1 eq) in DMSO (5 mL) was added 3-fluoro-4-(4-methylpiperazin-1-yl)aniline (4) (260 mg, 1.244 mmol, 1.2 eq) at room temperature. The reaction mixture was further heated at 100° C. for 16 h. The progress of the reaction was monitored by TLC (M.Ph: 5% Methanol in DCM). The reaction mixture was poured into ice cold water (250 mL) and extracted with ethyl acetate (2 × 100 mL). The combined organic layer was washed with ice cold water (2 × 50 mL) followed by brine (2 × 50 mL). The organic layer was separated, dried over anhydrous sodium sulfate, filtered and concentrated in vacuo resulting in the crude compound. The crude compound was purified through 230-400 mesh size silica gel column chromatography (elution: 0-5% methanol in DCM) to afford SSTN-572 (230 mg, 49%) as an off white solid. ¹H NMR (CDC1₃, 400 MHz): δ ppm 16.65 (s, 1H), 12.54 (s, 1H), 8.24-8.29 (m, 1H), 7.66-7.72 (m, 1H), 7.61 (dd, J=2.29, 14.24 Hz, 1H), 7.37 (d, J=8.65 Hz, 1H), 7.27-7.34 (m, 2H), 6.94 (t, J=9.03 Hz, 1H), 4.15-4.22 (m, 2H), 3.10-3.16 (m, 4H), 2.60-2.66 (m, 4H), 2.38 (s, 3H), 2.22-2.27 (m, 1H), 1.01 (d, J=6.61 Hz, 6H); LC-MS: m/z 453.10 [M+H]⁺; HPLC: 99.58%.

Synthesis ofN-(3-fluoro-4-(4-methylpiperazin-1-yl)phenyl)-4-hydroxy-1-isobutyl-2-oxo-1,2-dihydroquinoline-3-carboxamide hydrochloride salt (SSTN-572_HCl salt, Notch1 Reporter Assay IC₅₀: 3.4 µM)

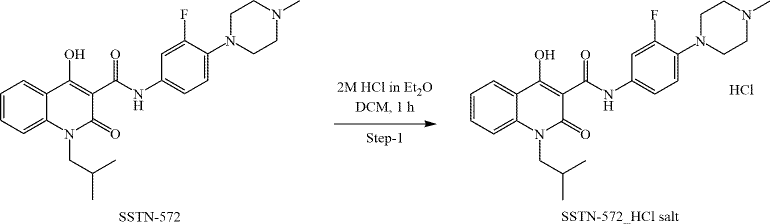

### Step-1: Synthesis of N-(3-Fluoro-4-(4-Methylpiperazin-1-yl)Phenyl)-4-Hydroxy-1-Isobutyl-2-Oxo-1,2-Dihydroquinoline-3-Carboxamide Hydrochloride Salt(SSTN-572_HCl Salt)

To a stirred mixture ofN-(3-fluoro-4-(4-methylpiperazin-1-yl)phenyl)-4-hydroxy-1-isobutyl-2-oxo-1,2-dihydroquinoline-3-carboxamide (SSTN-572) (20 mg, 0.044 mmol, 1 eq) in Et₂O (1 mL) at was added 2 M HCl in dioxane (100 µL). The reaction mixture was stirred for 1 h then concentrated in vacuo resulting in the crude compound. The crude compound was purified by trituration with Et₂O/DCM, filtered and dried under vacuum to afford SSTN-572_HC1 salt (17.6 mg, 82%) an off-white solid. ¹H NMR (400 MHz, DMSO) δ 10.54 (s, 1H), 8.14 (dd, J= 8.1, 1.6 Hz, 1H), 7.83 (ddd, J= 8.7, 7.0, 1.6 Hz, 1H), 7.76 - 7.66 (m, 2H), 7.45 - 7.33 (m, 2H), 7.14 (t, J= 9.2 Hz, 1H), 4.20 (d, J= 7.5 Hz, 2H), 3.48 (d, J= 11.7 Hz, 4H), 3.40 (s, 1H), 3.21 (d, J= 10.9 Hz, 2H), 3.10 (t, J= 12.0 Hz, 2H), 2.83 (d, J= 3.2 Hz, 3H), 2.17 (hept, J= 6.8 Hz, 1H), 0.92 (d, J= 6.7 Hz, 6H); LC-MS: m/z 453.2 [M+H]⁺.

SSTN-576

Synthesis of 6-bromo-4-hydroxy-1-isobutyl-N-(3-methylpyridin-2-yl)-2-oxo-1,2-dihydroquinoline-3-carboxamide (SSTN-576)

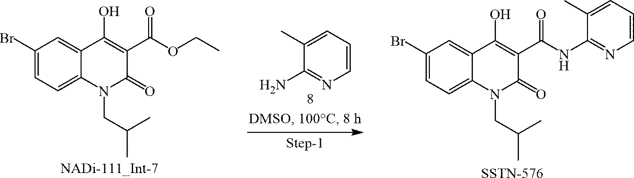

### Step-1: 6-Bromo-4-Hydroxy-1-Isobutyl-N-(3-Methylpyridin-2-yl)-2-Oxo-1,2-Dihydroquinoline-3-Carboxamide (SSTN-576)

To a stirred mixture of ethyl 6-bromo-4-hydroxy-1-isobutyl-2-oxo-1,2-dihydroquinoline-3-carboxylate (1.00 g, 2.715 mmol, 1 eq) in DMSO (10 mL) was added 3-fluoroaniline (8) (352 mg, 3.258 mmol, 1.5 eq) at room temperature. The reaction mixture was heated at 100° C. for 8 h. The progress of the reaction was monitored by TLC (M.Ph: 5% methanol in DCM). The reaction mixture was cooled to room temperature and poured over ice cold water (100 mL) and extracted with EtOAc (250 mL). The combined organic layer was washed with water (2 × 200 mL) followed by brine (2 × 200 mL). The organic layer was separated, dried over anhydrous sodium sulfate, filtered and concentrated in vacuo resulting in the crude compound. The crude compound was purified through 100-200 mesh size silica gel column chromatography (elution: 10-90% ethyl acetate in n-hexane) to afford SSTN-576 (550 mg, 47%) as white solid. ¹H NMR (DMSO-d₆, 400 MHz): δ ppm 16.73 (br. s, 1H), 12.40 (br. s, 1H), 8.33 (d, J=3.56 Hz, 1H), 8.21 (d, J=2.03 Hz, 1H), 7.92-7.98 (m, 1H), 7.79 (d, J=7.38 Hz, 1H), 7.71 (d, J=8.90 Hz, 1H), 7.30 (dd, J=4.70, 7.50 Hz, 1H), 4.19 (d, J=6.87 Hz, 2H), 2.30 (s, 3H), 2.15 (td, J=6.77, 13.67 Hz, 1H), 0.93 (d, J=6.61 Hz, 6H); LC-MS: m/z 431.90 [M+H]⁺; HPLC: 95.23%.

SSTN-577 (free base, HCl salt)

Synthesis of N-(3-fluorophenyl)-4-hydroxy-1-isobutyl-6-(4-methylpiperazin-1-yl)-2-oxo-1,2-dihydroquinoline-3-carboxamide (SSTN-577, Notch1 Reporter Assay IC₅₀: 9.69 µM)

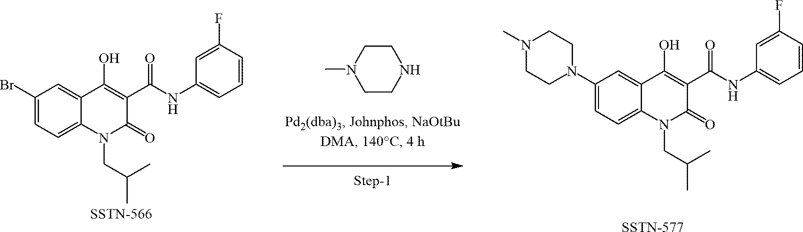

### Step-1: N-(3-Fluorophenyl)-4-Hydroxy-1-Isobutyl-6-(4-Methylpiperazin-1-yl)-2-Oxo-1,2-Dihydroquinoline-3-Carboxamide(SSTN-577)

To a stirred mixture of 6-bromo-N-(3-fluorophenyl)-4-hydroxy-1-isobutyl-2-oxo-1,2-dihydroquinoline-3-carboxamide (SSTN-566) (200 mg, 0.461 mmol, 1 eq) and 1-methylpiperazine (50.8 mg, 0.507 mmol, 1.1 eq) in DMA (5 mL) was added NaOtBu (88.6 mg, 0.923 mmol, 2 eq) and degassed with argon for 10-15 min at room temperature. To the resulting solution was added Pd₂(dba)₃ (42.2 mg, 0.046 mmol, 0.1 eq), Johnphos (20.4 mg, 0.069 mmol, 0.15 eq) and degassed with argon for another 10-15 min. The reaction mixture was further heated at 140° C. for 4 h. The progress of the reaction was monitored by TLC (M.Ph: 5% Methanol in DCM). The reaction mixture was cooled to room temperature, poured into ice cold water (50 mL) and extracted with ethyl acetate (2 × 100 mL). The combined organic layer was washed with water (3 × 20 mL) followed by brine (10 mL). The combined organic layer was dried over anhydrous sodium sulfate, filtered and concentrated in vacuo resulting in the crude compound. The crude compound was purified through 230-400 mesh size silica gel column chromatography (elution: 2-8% methanol in DCM) to afford SSTN-577 (82 mg, 39.4%) as yellow solid. ¹H NMR (CDC1₃, 400 MHz): δ ppm 16.51 (s, 1H), 12.87 (s, 1H), 7.65 (d, J=2.45 Hz, 2H), 7.27-7.40 (m, 4H), 6.82-6.89 (m, 1H), 4.12-4.19 (m, 2H), 3.26-3.31 (m, 4H), 2.60-2.66 (m, 4H), 2.38 (s, 3H), 2.17-2.28 (m, 1H), 1.00 (d, J=6.85 Hz, 6H); LC-MS: m/z 453.15 [M+H]⁺; HPLC: 99.42%.

Synthesis of N-(3 -fluorophenyl)-4-hydroxy-1 -isobutyl-6-(4-methylpiperazin-1 -yl)-2-oxo-1,2-dihydroquinoline-3-carboxamide hydrochloride salt (SSTN-577_HCl salt, Notch1 Reporter Assay IC₅₀: 6.44 µM)

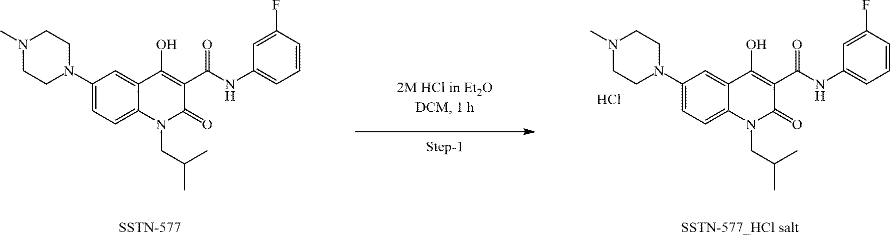

### Step-1: Synthesis of N-(3-Fluorophenyl)-4-Hydroxy-1-Isobutyl-6-(4-Methylpiperazin-1-yl)-2-Oxo-1,2-Dihydroquinoline-3-Carboxamide Hydrochloride Salt (SSTN-577_HCl Salt)

To a stirred mixture of N-(3-fluorophenyl)-4-hydroxy-1-isobutyl-6-(4-methylpiperazin-1-yl)-2-oxo-1,2-dihydroquinoline-3-carboxamide (SSTN-577) (20 mg, 0.044 mmol, 1 eq) in Et₂O (1 mL) at was added 2 M HCl in dioxane (100 µL). The reaction mixture was stirred for 1 h then concentrated in vacuo resulting in the crude compound. The crude compound was purified by trituration with Et₂O/DCM, filtered and dried under vacuum to afford SSTN-577_HCl salt (16.3 mg, 76%) an off-white solid. ¹H NMR (400 MHz, DMSO) δ 10.31 (s, 1H), 7.75 - 7.65 (m, 2H), 7.62 (dd, J= 9.4, 2.9 Hz, 1H), 7.52 (d, J= 2.8 Hz, 1H), 7.49 - 7.35 (m, 2H), 7.08 - 6.98 (m, 1H), 4.19 (d, J= 7.6 Hz, 2H), 3.91 (d, J= 12.6 Hz, 2H), 3.53 (d, J= 11.7 Hz, 2H), 3.41 (s, 1H), 3.25 - 3.15 (m, 2H), 3.09 (t, J= 12.3 Hz, 2H), 2.87 - 2.82 (m, 3H), 2.15 (dt, J= 13.8, 6.9 Hz, 1H), 0.91 (d, J= 6.7 Hz, 6H); LC-MS: m/z 453.2 [M+H]⁺.

SSTN-578

Synthesis ofN-(3-fluorophenyl)-4-hydroxy-1-isobutyl-7-(4-methylpiperazin-1-yl)-2-oxo-1,2-dihydroquinoline-3-carboxamide (SSTN-578, Notch1 Reporter Assay IC₅₀: n/a)

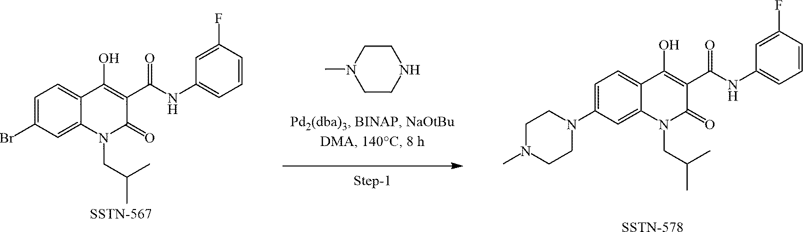

### Step-1: N-(3-Fluorophenyl)-4-Hydroxy-1-Isobutyl-7-(4-Methylpiperazin-1-yl)-2-Oxo-1,2-Dihydroquinoline-3-Carboxamide (SSTN-578)

To a stirred mixture of 7-bromo-N-(3-fluorophenyl)-4-hydroxy-1-isobutyl-2-oxo-1,2-dihydroquinoline-3-carboxamide (SSTN-567) (170 mg, 0.392 mmol, 1 eq), 1-methylpiperazine (47.1 mg, 0.470 mmol, 1.2 eq) and NaOtBu (75.3 mg, 0.784 mmol, 2 eq) in DMA (5 mL) was degassed with argon for 15 min. To the resulting solution was added Pd₂(dba)₃ (35.9 mg, 0.039 mmol, 0.1 eq), BINAP (36.6 mg, 0.058 mmol, 0.015 eq) at room temperature and degassed with argon for another 15 min. The reaction mixture was further heated at 140° C. for 8 h. The progress of the reaction was monitored by TLC (M.Ph: 5% Methanol in DCM). The reaction mixture was diluted with EtOAc (20 mL) and filtered through a Celite bed. The filtrate was dried over anhydrous sodium sulfate, filtered and concentrated in vacuo resulting in the crude compound. The crude compound from batch no. E20058-040 (30 mg scale) was combined after work-up for purification with batch no. E20058-041 (170 mg scale). The crude compound from both batches were combined purified through 230-400 mesh size silica gel column chromatography (elution: 0-5% methanol in DCM) to afford SSTN-578 (80 mg, 38.3%) as an off white solid. ¹H NMR (DMSO-d₆, 400 MHz): δ ppm 16.07 (br. s, 1H), 12.81 (br. s, 1H), 7.92 (d, J=9.16 Hz, 1H), 7.69 (d, J=11.19 Hz, 1H), 7.39-7.47 (m, 1H), 7.34-7.38 (m, 1H), 7.08 (d, J=9.41 Hz, 1H), 7.00 (t, J=7.88 Hz, 1H), 6.74 (s, 1H), 4.20 (d, J=5.85 Hz, 2H), 3.44-3.49 (m, 4H), 2.45-2.48 (m, 4H), 2.24 (s, 3H), 2.14-2.21 (m, 1H), 0.93 (d, J=6.61 Hz, 6H); LC-MS: m/z 453.10 [M+H]⁺; HPLC: 99.67%.

Synthesis ofN-(3-fluorophenyl)-4-hydroxy-1-isobutyl-7-(4-methylpiperazin-1-yl)-2-oxo-1,2-dihydroquinoline-3-carboxamide hydrochloride salt (SSTN-578_HCl salt, Notch1 Reporter Assay IC₅₀: n/a)

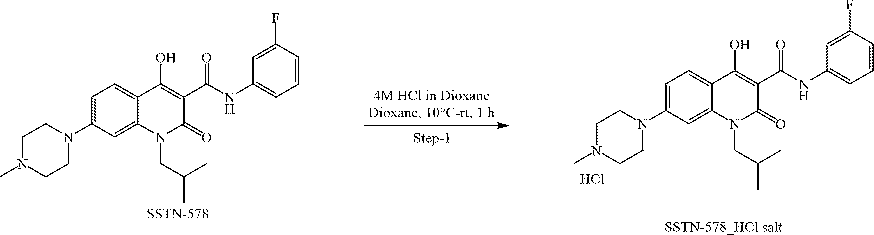

### Step-1: N-(3-Fluorophenyl)-4-Hydroxy-1-Isobutyl-7-(4-Methylpiperazin-1-yl)-2-Oxo-1,2-Dihydroquinoline-3-Carboxamide Hydrochloride Salt (SSTN-585_HCl Salt)

To a stirred mixture of N-(3-fluorophenyl)-4-hydroxy-1-isobutyl-7-(4-methylpiperazin-1-yl)-2-oxo-1,2-dihydroquinoline-3-carboxamide (SSTN-578) (45 mg, 0.099 mmol, 1 eq) in dioxane (5 mL) at 10° C. was added 4 M HCl in dioxane (1 mL). The reaction mixture was allowed to attain at room temperature and stirred for 1 h. The progress of the reaction was monitored by TLC (M.Ph: 5% Methanol in DCM). The reaction mixture was concentrated in vacuo resulting in the crude compound. The crude compound was purified by trituration with n-hexane (5 mL), filtered and dried under vacuum to afford SSTN-578_HCl salt (38.6 mg, 79.5%) as an off white solid. ¹H NMR (DMSO-d₆, 400 MHz): δ ppm 16.14 (s, 1H), 12.78 (s, 1H), 10.58 (br. s, 1H), 7.99 (d, J=9.29 Hz, 1H), 7.69 (d, J=11.25 Hz, 1H), 7.40-7.48 (m, 1H), 7.34-7.40 (m, 1H), 7.14 (d, J=9.29 Hz, 1H), 6.98-7.06 (m, 1H), 6.85 (s, 1H), 4.23 (d, J=8.80 Hz, 4H), 3.49-3.59 (m, 4H), 3.16 (d, J=9.78 Hz, 2H), 2.84 (br. s, 3H), 2.19 (td, J=6.79, 13.33 Hz, 1H), 0.94 (d, J=6.85 Hz, 6H); LC-MS: m/z 453.50 [M+H]⁺; HPLC: 99.34%.

SSTN-579 (free base, HCl salt)

Synthesis of 4-hydroxy-1-isobutyl-6-(4-methylpiperazin-1-yl)-N-(3-methylpyridin-2-yl)-2-oxo-1,2-dihydroquinoline-3-carboxamide (SSTN-579, Notch1 Reporter Assay IC₅₀: 18.4 µM)

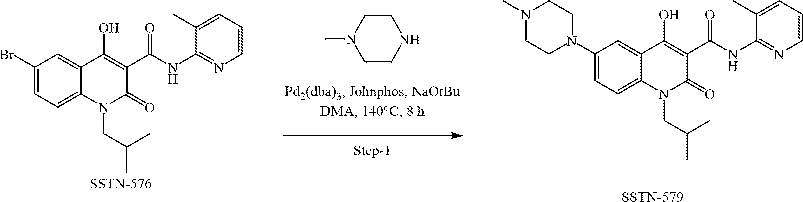

### Step-1: 4-Hydroxy-1-Isobutyl-6-(4-Methylpiperazin-1-yl)-N-(3-Methylpyridin-2-yl)-2-Oxo-1,2-Dihydroquinoline-3-Carboxamide (SSTN-579)

To a stirred mixture of 6-bromo-4-hydroxy-1-isobutyl-N(3-methylpyridin-2-yl)-2-oxo-1,2-dihydroquinoline-3-carboxamide (SSTN-576) (200 mg, 0.464 mmol, 1 eq) and 1-methylpiperazine (51.2 mg, 0.511 mmol, 1.1 eq) in DMA (5 mL) was added NaOtBu (89.1 mg, 0.928 mmol, 2 eq) and degassed with argon for 10-15 min at room temperature. To the resulting solution was added Pd₂(dba)₃ (42.4 mg, 0.046 mmol, 0.1 eq), Johnphos (20.6 mg, 0.069 mmol, 0.15 eq) and degassed with argon for another 10-15 min. The reaction mixture was further heated at 140° C. for 8 h in a sealed tube. The progress of the reaction was monitored by TLC (M.Ph: 5% Methanol in DCM). The reaction mixture was poured into ice cold water (50 mL) and extracted with ethyl acetate (2 × 100 mL). The combined organic layer was washed with water (2 × 50 mL) followed by brine (2 × 50 mL). The combined organic layer was dried over anhydrous sodium sulfate, filtered and concentrated in vacuo resulting in the crude compound. The crude compound was purified through 230-400 mesh size silica gel column chromatography (elution: 2-8% methanol in DCM) to afford SSTN-579 (25 mg, 12%) as yellow solid. ¹H NMR (CDC1₃, 400 MHz): δ ppm 16.59 (br. s, 1H), 12.90 (s, 1H), 8.42 (d, J=3.91 Hz, 1H), 7.66 (d, J=2.45 Hz, 1H), 7.58 (d, J=7.34 Hz, 1H), 7.34-7.38 (m, 1H), 7.28-7.32 (m, 1H), 7.10 (dd, J=4.89, 7.34 Hz, 1H), 4.13-4.21 (m, 2H), 3.31-3.37 (m, 4H), 2.69-2.77 (m, 4H), 2.46 (s, 3H), 2.41 (s, 3H), 2.17-2.27 (m, 1H), 1.00 (d, J=6.85 Hz, 6H). LC-MS: m/z 450.10 [M+H]⁺; HPLC: 99.20%.

Synthesis of 4-hydroxy-1-isobutyl-6-(4-methylpiperazin-1-yl)-N-(3-methylpyridin-2-yl)-2-oxo-1,2-dihydroquinoline-3-carboxamide hydrochloride salt (SSTN-579_HCl salt, Notch1 Reporter Assay IC₅₀: n/a)

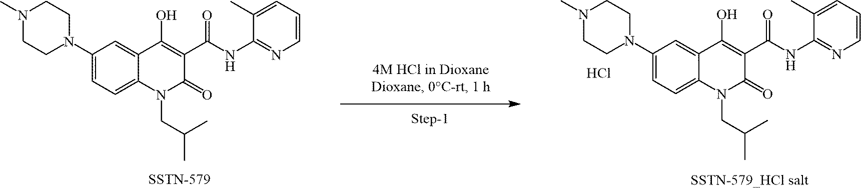

### Step-1: 4-Hydroxy-1-Isobutyl-6-(4-Methylpiperazin-1-yl)-N-(3-Methylpyridin-2-yl)-2-Oxo-1,2-Dihydroquinoline-3-Carboxamide Hydrochloride Salt (SSTN-579_HCl Salt)

To a stirred mixture of 4-hydroxy-1-isobutyl-6-(4-methylpiperazin-1-yl)-N-(3-methylpyridin-2-yl)-2-oxo-1,2-dihydroquinoline-3-carboxamide (SSTN-579) (7 mg, 0.015 mmol, 1 eq) in dioxane (2 mL) at 0° C. was added 4 M HCl in dioxane (0.3 mL). The reaction mixture was allowed to attain at room temperature and stirred for 1 h. The progress of the reaction was monitored by TLC (M.Ph: 5% Methanol in DCM). The reaction mixture was concentrated in vacuo resulting in the crude compound. The crude compound was purified by trituration with n-hexane (5 mL), filtered and dried under vacuum to afford SSTN-579_HCl salt (7 mg, 93.3%) as yellow solid. ¹H NMR (DMSO-d₆, 400 MHz): δ ppm 16.43 (br. s, 1H), 12.87 (br. s, 1H), 10.44 (br. s, 1H), 8.35 (d, J=2.93 Hz, 1H), 7.88 (d, J=5.87 Hz, 1H), 7.61-7.73 (m, 2H), 7.55 (br. s, 1H), 7.30-7.37 (m, 1H), 4.17-4.25 (m, 2H), 4.02-4.14 (m, 2H), 3.92 (d, J=11.74 Hz, 2H), 3.03-3.27 (m, 4H), 2.85 (br. s, 3H), 2.33 (br. s, 3H), 2.07-2.21 (m, 1H), 0.93 (d, J=5.87 Hz, 6H). LC-MS: m/z 448.30 [M-H]⁺; HPLC: 98.00%.

SSTN-580 (TFA salt, HCl salt)

Synthesis of 4-hydroxy-1-isobutyl-7-(4-methylpiperazin-1-yl)-N-(3-methylpyridin-2-yl)-2-oxo-1,2-dihydroquinoline-3-carboxamide SSTN-580_TFA salt, Notch1 Reporter Assay IC₅₀: 13.3 µM)

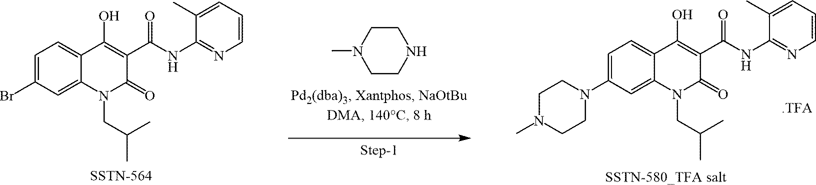

### Step-1: 4-Hydroxy-1-Isobutyl-7-(4-Methylpiperazin-1-yl)-N-(3-Methylpyridin-2-yl)-2-Oxo-1,2-Dihydroquinoline-3-Carboxamide (SSTN-580_TFA Salt)

To a stirred mixture of 7-bromo-4-hydroxy-1-isobutyl-N-(3-methylpyridin-2-yl)-2-oxo-1,2-dihydroquinoline-3-carboxamide (SSTN-564) (150 mg, 0.348 mmol, 1 eq) and 1-methylpiperazine (41.9 mg, 0.418 mmol, 1.2 eq) in DMA (5 mL) was added NaOtBu (66.9 mg, 0.697 mmol, 1.2 eq) and degassed with argon for 10-15 min at room temperature. To the resulting solution was added Pd₂(dba)₃ (31.9 mg, 0.034 mmol, 0.1 eq), Xantphos (30.2 mg, 0.052 mmol, 1.2 eq) and degassed with argon for another 10-15 min. The reaction mixture was further heated at 140° C. for 8 h. The progress of the reaction was monitored by TLC (M.Ph: 5% Methanol in DCM). The reaction mixture was poured into ice cold water (50 mL) and extracted with ethyl acetate (2 × 50 mL). The combined organic layer was washed with water (2 × 50 mL) followed by brine (2 × 50 mL). The combined organic layer was dried over anhydrous sodium sulfate, filtered and concentrated in vacuo resulting in the crude compound. The crude compound was purified through 230-400 mesh size silica gel column chromatography (elution: 0-5% methanol in DCM). Compound obtained after column purification was repurified by preparative HPLC to afford SSTN-580_TFA salt (40 mg, 20.4%) as an off white solid. ¹H NMR (DMSO-d₆, 400 MHz): δ ppm 16.30 (br. s, 1H), 12.54 (br. s, 1H), 9.80 (br. s, 1H), 8.32 (d, J=3.91 Hz, 1H), 7.99 (d, J=9.29 Hz, 1H), 7.81 (d, J=7.34 Hz, 1H), 7.30 (dd, J=4.89, 7.34 Hz, 1H), 7.14 (d, J=9.29 Hz, 1H), 6.85 (br. s, 1H), 4.24 (d, J=7.34 Hz, 4H), 3.11-3.29 (m, 6H), 2.89 (br. s, 3H), 2.30 (s, 3H), 2.13-2.25 (m, 2H), 0.95 (d, J=6.85 Hz, 6H). LC-MS: m/z 450.20 [M+H]⁺; HPLC: 97.75%.

Synthesis of 4-hydroxy-1-isobutyl-7-(4-methylpiperazin-1-yl)-N-(3-methylpyridin-2-yl)-2-oxo-1,2-dihydroquinoline-3-carboxamide hydrochloride salt (SSTN-580_HCl salt, Notch1 Reporter Assay IC₅₀: 14.0 µM)

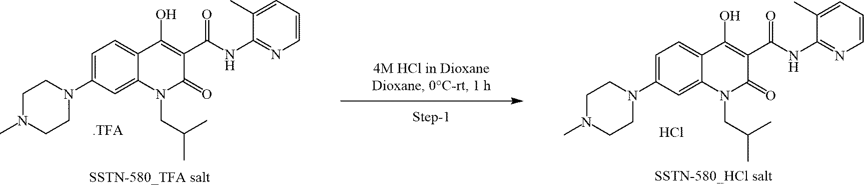

### Step-1: 4-Hydroxy-1-Isobutyl-7-(4-Methylpiperazin-1-yl)-N-(3-Methylpyridin-2-yl)-2-Oxo-1,2-Dihydroquinoline-3-Carboxamide Hydrochloride Salt (SSTN-580_HCl Salt)

To a stirred mixture of 4-hydroxy-1-isobutyl-7-(4-methylpiperazin-1-yl)-N-(3-methylpyridin-2-yl)-2-oxo-1,2-dihydroquinoline-3-carboxamide TFA salt (SSTN-580_TFA salt) (20 mg, 0.035 mmol, 1 eq) in dioxane (2 mL) at 10° C. was added 4 M HCl in dioxane (0.5 mL). The reaction mixture was allowed to attain room temperature and stirred for 1 h. The progress of the reaction was monitored by TLC (M.Ph: 5% methanol in DCM). The reaction mixture was concentrated in vacuo resulting in the crude compound. The crude compound was purified by trituration with n-hexane (5 mL), filtered and dried under vacuum to afford SSTN-580_HCl salt (9.5 mg, 55.8%) as yellow solid. ¹H NMR (DMSO-d₆, 400 MHz): δ ppm 15.98 (br. s, 1H), 12.84 (br. s, 1H), 10.76 (br. s, 1H), 8.35 (br. s, 1H), 8.00 (d, J=8.80 Hz, 1H), 7.93 (d, J=7.34 Hz, 1H), 7.38 (d, J=4.89 Hz, 1H), 7.16 (d, J=8.80 Hz, 1H), 6.86 (br. s, 1H), 4.25 (d, J=7.34 Hz, 3H), 3.54 (d, J=11.74 Hz, 3H), 3.31-3.42 (m, 2H), 3.10-3.24 (m, 2H), 2.84 (br. s, 3H), 2.34 (br. s, 3H), 2.15-2.25 (m, 1H), 0.95 (d, J=6.85 Hz, 6H); LC-MS: m/z 448.00 [M-H]⁺; HPLC: 96.29%.

SSTN-581 (formate salt, HCl salt)

Synthesis of N-(3 -fluoro-2-(4-methylpiperazin-1 -yl)phenyl)-4-hydroxy-1 -isobutyl-2-oxo-1,2-dihydroquinoline-3-carboxamide formate salt (SSTN-581_Formate salt, Notch1 Reporter Assay IC₅₀: 7.76 µM)

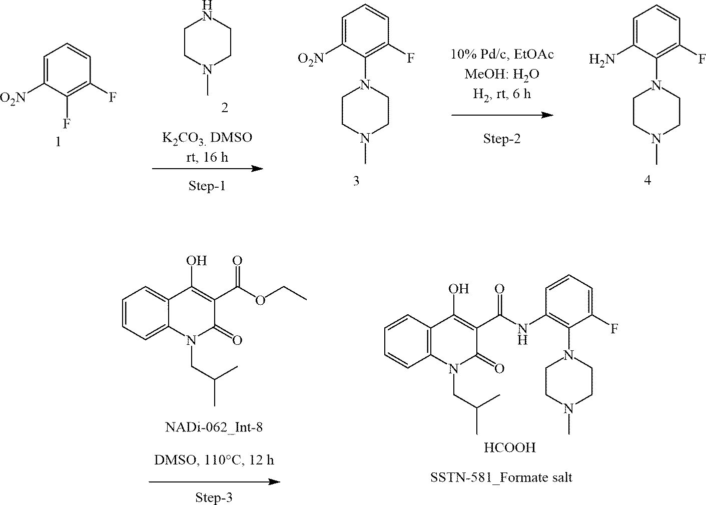

### Step-1: 1-(2-Fluoro-6-Nitrophenyl)-4-Methylpiperazine (3)

To a stirred mixture of 1,2-difluoro-3-nitrobenzene (1) (1.00 g, 6.285 mmol, 1 eq) and 1-methylpiperazine (2) (692 mg, 6.913 mmol, 1.2 eq) in DMSO (10 mL) was added potassium carbonate (1.73 g, 12.57 mmol, 3 eq) at room temperature and stirred for 16 h. The reaction was monitored by TLC (M.Ph: 5% methanol in DCM). The reaction mixture was poured into ice cold water (50 mL) and extracted with EtOAc (2 × 100 mL). The organic layer was washed with ice cold water (2 × 50 mL) followed by brine (2 × 50 mL). The organic layer was separated, dried over anhydrous sodium sulfate, filtered and concentrated in vacuo resulting in the crude compound. The crude compound was purified by trituration with diethyl ether (20 mL) and n-hexane (30 mL), filtered and dried under vacuum to afford 3 (1.40 g, 93.3%) as brown solid. ¹H NMR (DMSO-d₆, 400 MHz): δ ppm 7.61 (d, J=8.31 Hz, 1H), 7.52 (dd, J=8.31, 12.23 Hz, 1H), 7.32 (dt, J=5.14, 8.19 Hz, 1H), 3.02 (t, J=4.16 Hz, 4H), 2.33-2.39 (m, 4H), 2.20 (s, 3H); LC-MS: m/z 239.72 [M+H]⁺.

### Step-2: 3-Fluoro-2-(4-Methylpiperazin-1-yl)Aniline (4)

To a stirred solution of 1-(2-fluoro-6-nitrophenyl)-4-methylpiperazine (3) (700 mg, 2.925 mmol, 1 eq) in MeOH (5 mL) was added slurry of 10% Pd/C (300 mg) in ethyl acetate (3 mL) followed by water (1 mL) at room temperature into a hydrogenator. The mixture was degassed for 15 min with the help of alternative vacuum and nitrogen. The reaction was stirred under hydrogen atmosphere for 6 h at room temperature. The reaction was monitored by TLC (M.Ph: 5% methanol in DCM). The reaction mixture was filtered through Celite bed and the filtrate was concentrated in vacuo resulting in the crude compound. The crude compound was purified by trituration with diethyl ether and n-hexane, filtered and dried under vacuum to afford 4 (550 mg, 89.8%) as brown solid. ¹H NMR (DMSO-d₆, 400 MHz): δ ppm 6.78-6.86 (m, 1H), 6.46 (d, J=7.82 Hz, 1H), 6.25 (dd, J=8.07, 12.47 Hz, 1H), 5.17 (br. s, 2H), 2.96-3.20 (m, 2H), 2.59-2.85 (m, 3H), 2.21 (s, 3H); LC-MS: m/z 210.15 [M+H]⁺.

### Step-3: N-(3-Fluoro-2-(4-Methylpiperazin-1-yl)Phenyl)-4-Hydroxy-1-Isobutyl-2-Oxo-1,2-Dihydroquinoline-3-Carboxamide Formate Salt(SSTN-581_Formate Salt)

To a stirred mixture of ethyl 4-hydroxy-1-isobutyl-2-oxo-1,2-dihydroquinoline-3-carboxylate (300 mg, 1.036 mmol, 1 eq) in DMSO (5 mL) was added 3-fluoro-2-(4-methylpiperazin-1-yl)aniline (4) (260 mg, 1.244 mmol, 1.2 eq) at room temperature. The reaction mixture was further heated at 110° C. for 12 h. The progress of the reaction was monitored by TLC (M.Ph: 5% Methanol in DCM). The reaction mixture was poured into ice cold water (50 mL) and extracted with ethyl acetate (2 × 50 mL). The combined organic layer was washed with ice cold water (2 × 50 mL) followed by brine (2 × 50 mL). The organic layer was separated, dried over anhydrous sodium sulfate, filtered and concentrated in vacuo resulting in the crude compound. The crude compound was purified through 230-400 mesh size silica gel column chromatography (elution: 0-5% methanol in DCM). The compound obtained after column purification was repurified by preparative HPLC to afford SSTN-581_Formate salt (160 mg, 30.9%) as an off white solid. ¹H NMR (DMSO-d₆, 400 MHz): δ ppm 13.45 (s, 1H), 8.27 (d, J=8.31 Hz, 1H), 8.13-8.18 (m, 2H), 7.80-7.86 (m, 1H), 7.71 (d, J=8.80 Hz, 1H), 7.40 (t, J=7.58 Hz, 1H), 7.24-7.31 (m, 1H), 7.01 (dd, J=8.56, 11.98 Hz, 1H), 4.24 (d, J=7.34 Hz, 2H), 2.85-3.08 (m, 4H), 2.65-2.73 (m, 4H), 2.33 (s, 3H), 2.18-2.28 (m, 2H), 0.99 (d, J=6.85 Hz, 6H); LC-MS: m/z 453.10 [M+H]⁺; HPLC: 99.86%.

Synthesis of N-(3 -fluoro-2-(4-methylpiperazin-1 -yl)phenyl)-4-hydroxy-1 -isobutyl-2-oxo-1,2-dihydroquinoline-3-carboxamide hydrochloride salt (SSTN-581_HCl salt, Notch1 Reporter Assay IC₅₀: 7.68 µM)

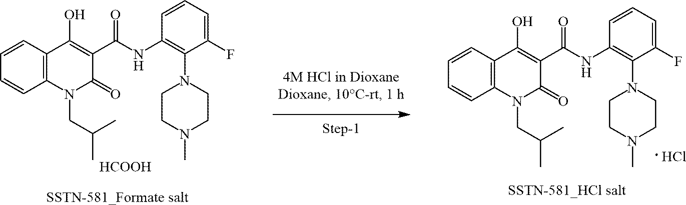

### Step-1: N-(3-Fluoro-2-(4-Methylpiperazin-1-yl)Phenyl)-4-Hydroxy-1-Isobutyl-2-Oxo-1,2-Dihydroquinoline-3-Carboxamide Hydrochloride Salt (SSTN-581_HCl Salt)

To a stirred mixture of N-(3-fluoro-2-(4-methylpiperazin-1-yl)phenyl)-4-hydroxy-1-isobutyl-2-oxo-1,2-dihydroquinoline-3-carboxamide formate salt (SSTN-581_Formate salt) (45 mg, 0.090 mmol, 1 eq) in dioxane (3 mL) at 10° C. was added 4 M HCl in dioxane (1 mL). The reaction mixture was allowed to attain room temperature and stirred for 1 h. The progress of the reaction was monitored by TLC (M.Ph: 5% methanol in DCM). The reaction mixture was concentrated in vacuo resulting in the crude compound. The crude compound was purified by trituration with n-hexane (5 mL), filtered and dried under vacuum to afford SSTN-581_HCl salt (38 mg, 93.3%) as an off white solid. ¹H NMR (DMSO-d₆, 400 MHz): δ ppm 16.61 (br. s, 1H), 13.45 (br. s, 1H), 10.36-10.56 (m, 1H), 8.30 (d, J=7.82 Hz, 1H), 8.18 (d, J=7.34 Hz, 1H), 7.85 (t, J=7.34 Hz, 1H), 7.76 (d, J=8.31 Hz, 1H), 7.43 (t, J=7.09 Hz, 1H), 7.35 (d, J=7.34 Hz, 1H), 7.01-7.12 (m, 1H), 4.21-4.32 (m, 2H), 3.56-3.64 (m, 2H), 3.38-3.55 (m, 4H), 3.15-3.23 (m, 2H), 2.91 (br. s, 3H), 2.16-2.27 (m, 1H), 0.98 (d, J=5.87 Hz, 6H); LC-MS: m/z 453.15 [M+H]⁺; HPLC: 99.75%.

SSTN-582 (free base, HCl salt)

Synthesis ofN-(3,5-difluoro-4-(4-methylpiperazin-1-yl)phenyl)-4-hydroxy-1-isobutyl-2-oxo-1,2-dihydroquinoline-3-carboxamide (SSTN-582, Notch1 Reporter Assay IC₅₀: n/a)

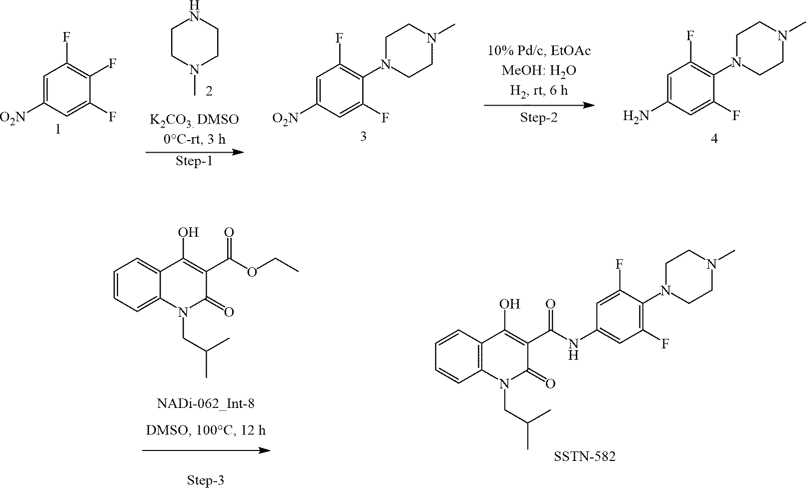

### Step-1: 1-(2,6-Difluoro-4-Nitrophenyl)-4-Methylpiperazine (3)

To a stirred mixture of 1,2,3-trifluoro-5-nitrobenzene (1) (1.00 g, 5.646 mmol, 1 eq) and 1-methylpiperazine (2) (621 mg, 6.211 mmol, 1.1 eq) in DMSO (10 mL) at 0° C. was added potassium carbonate (1.56 g, 11.29 mmol, 2 eq). Reaction mixture was allowed to attain room temperature and stirred for 3 h. The reaction was monitored by TLC (M.Ph: 5% methanol in DCM). The reaction mixture was poured into ice cold water (300 mL) and extracted with ethyl acetate (2 × 200 mL). The combined organic layer was washed with water (2 × 200 mL) followed by brine (2 × 200 mL). The organic layer was separated, dried over anhydrous sodium sulfate, filtered and concentrated in vacuo to afford 3 (1.40 g, 96.5%) as brown solid. Crude compound obtained was used as such in the next step without further purification. ¹H NMR (CDCl₃, 400 MHz): δ ppm 7.76 (d, J=9.16 Hz, 2H), 3.38-3.43 (m, 4H), 2.51-2.57 (m, 4H), 2.35 (s, 3H); LC-MS: m/z 258.10 [M+H]⁺.

### Step-2: 3,5-Difluoro-4-(4-Methylpiperazin-1-yl)Aniline (4)

To a stirred solution of 1-(2,6-difluoro-4-nitrophenyl)-4-methylpiperazine (3) (700 mg, 2.721 mmol, 1 eq) in MeOH (5 mL) was added slurry of 10% Pd/C (300 mg) in ethyl acetate (3 mL) followed by water (1 mL) at room temperature into a hydrogenator. The mixture was degassed for 15 min with the help of alternative vacuum and nitrogen. The reaction was stirred under hydrogen atmosphere for 6 h at room temperature. The reaction was monitored by TLC (M.Ph: 5% methanol in DCM). The reaction mixture was filtered through Celite bed and the filtrate was concentrated in vacuo resulting in the crude compound. The crude compound was purified by trituration with diethyl ether and n-hexane, filtered and dried under vacuum to afford 4 (600 mg, 97%) as brown solid. ¹H NMR (DMSO-d₆, 400 MHz): δ ppm 6.13 (d, J=11.74 Hz, 2H), 5.43 (s, 2H), 2.88-2.94 (m, 4H), 2.32-2.38 (m, 4H), 2.18 (s, 3H); LC-MS: m/z 228.01 [M+H]⁺.

### Step-3: N-(3,5-Difluoro-4-(4-Methylpiperazin-1-yl)Phenyl)-4-Hydroxy-1-Isobutyl-2-Oxo-1,2-Dihydroquinoline-3-Carboxamide (SSTN-582)

To a stirred mixture of ethyl 4-hydroxy-1-isobutyl-2-oxo-1,2-dihydroquinoline-3-carboxylate (250 mg, 0.864 mmol, 1 eq) in DMSO (5 mL) was added 3,5-difluoro-4-(4-methylpiperazin-1-yl)aniline (4) (235 mg, 1.036 mmol, 1.2 eq) at room temperature. The reaction mixture was further heated at 100° C. for 12 h. The progress of the reaction was monitored by TLC (M.Ph: 5% Methanol in DCM). The reaction mixture was poured into ice cold water (250 mL) and extracted with ethyl acetate (2 × 100 mL). The combined organic layer was washed with ice cold water (2 × 50 mL) followed by brine (2 × 50 mL). The organic layer was separated, dried over anhydrous sodium sulfate, filtered and concentrated in vacuo resulting in the crude compound. The crude compound was purified through 230-400 mesh size silica gel column chromatography (elution: 0-2% methanol in DCM) to afford SSTN-582 (65 mg, 16%) as a pale yellow solid. ¹H NMR (CDCl₃, 400 MHz): δ ppm 16.28 (s, 1H), 12.73 (s, 1H), 8.27 (d, J=7.82 Hz, 1H), 7.70 (t, J=7.83 Hz, 1H), 7.38 (d, J=8.80 Hz, 1H), 7.29-7.35 (m, 3H), 4.18 (br. s, 2H), 3.37-3.47 (m, 4H), 2.84-2.97 (m, 4H), 2.60 (s, 3H), 2.18-2.30 (m, 2H), 1.01 (d, J=6.85 Hz, 6H); LC-MS: m/z 471.00 [M+H]⁺; HPLC: 99.39%.

Synthesis of N-(3,5-difluoro-4-(4-methylpiperazin-1-yl)phenyl)-4-hydroxy-1-isobutyl-2-oxo-1,2-dihydroquinoline-3-carboxamide hydrochloride salt (SSTN-582_HCl salt, Notch1 Reporter Assay IC₅₀: 1.90 µM)

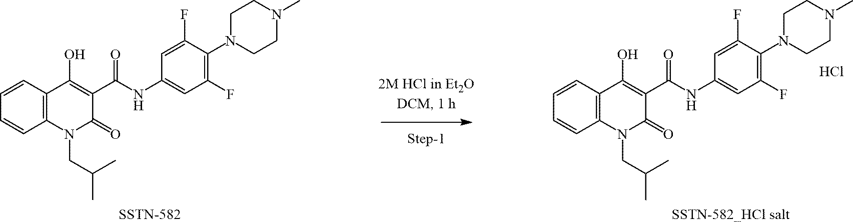

### Step-1: Synthesis of N-(3,5-Difluoro-4-(4-Methylpiperazin-1-yl)Phenyl)-4-Hydroxy-1-Isobutyl-2-Oxo-1,2-Dihydroquinoline-3-Carboxamide Hydrochloride Salt(SSTN-582_HCl Salt)

To a stirred mixture of N-(3,5-difluoro-4-(4-methylpiperazin-1-yl)phenyl)-4-hydroxy-1-isobutyl-2-oxo-1,2-dihydroquinoline-3-carboxamide (SSTN-582) (20 mg, 0.044 mmol, 1 eqv) in Et₂O (1 mL) at was added 2 M HCl in dioxane (100 µL). The reaction mixture was stirred for 1 h then concentrated in vacuo resulting in the crude compound. The crude compound was purified by trituration with Et₂O/DCM, filtered and dried under vacuum to afford SSTN-582_HCl salt (16.3 mg, 76%) an off-white solid. ¹H NMR (400 MHz, DMSO) δ 10.40 (s, 1H), 8.15 (dd, J= 8.1, 1.6 Hz, 1H), 7.84 (ddd, J= 8.7, 7.0, 1.6 Hz, 1H), 7.74 (d, J= 8.7 Hz, 1H), 7.56 - 7.45 (m, 2H), 7.41 (t, J= 7.5 Hz, 1H), 4.20 (d, J= 7.5 Hz, 2H), 3.53 - 3.36 (m, 6H), 3.32 (s, 1H), 3.22 - 3.11 (m, 2H), 2.83 (d, J= 3.2 Hz, 3H), 2.17 (hept, J= 6.8 Hz, 1H), 0.92 (d, J= 6.6 Hz, 6H); LC-MS: m/z 471.0 [M+H]⁺.

SSTN-583 (free base, HCl salt)

Synthesis ofN-(3-fluoro-5-(4-methylpiperazin-1-yl)phenyl)-4-hydroxy-1-isobutyl-2-oxo-1,2-dihydroquinoline-3-carboxamide (SSTN-583, Notch1 Reporter Assay IC₅₀: 8.13 µM)

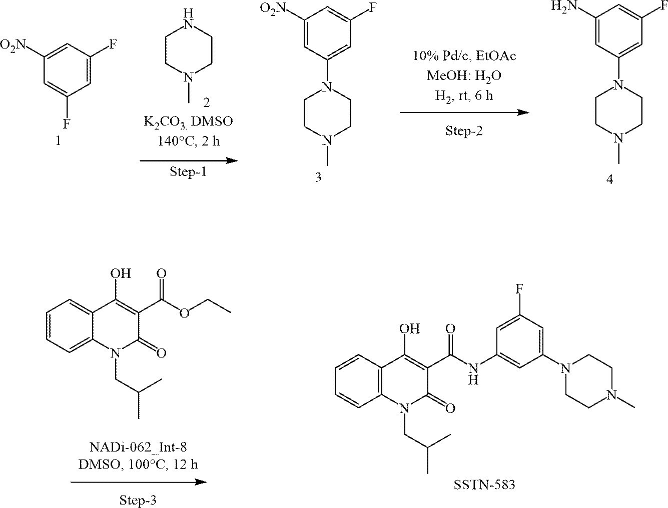

### Step-1: 1-(3-Fluoro-5-Nitrophenyl)-4-Methylpiperazine (3)

To a stirred mixture of 1,3-difluoro-5-nitrobenzene (1) (1.00 g, 6.285 mmol, 1 eq) and 1-methylpiperazine (2) (629 mg, 6.285 mmol, 1.2 eq) in DMSO (10 mL) was added potassium carbonate (1.73 g, 12.57 mmol, 3 eq) at room temperature. The reaction mixture was further heated at 140° C. for 2 h. The reaction was monitored by TLC (M.Ph: 20% ethyl acetate in n-hexane). The reaction mixture was poured into ice cold water (50 mL) and extracted with EtOAc (3 × 100 mL). The combined organic layer was washed with water (3 × 50 mL) followed by brine (10 mL). The organic layer was separated, dried over anhydrous sodium sulfate, filtered and concentrated in vacuo resulting in the crude compound. The crude compound was purified by trituration with diethyl ether (20 mL) and n-hexane (30 mL), filtered and dried under vacuum to afford 3 (1.00 g, 66.5%) as brown solid. LC-MS: m/z 239.90 [M+H]⁺.

### Step-2: 3-Fluoro-5-(4-Methylpiperazin-1-yl)Aniline (4)

To a stirred solution of 1-(3-fluoro-5-nitrophenyl)-4-methylpiperazine (3) (550 mg, 2.298 mmol, 1 eq) in MeOH (5 mL) was added slurry of 10% Pd/C (250 mg) in ethyl acetate (3 mL) followed by water (1 mL) at room temperature into a hydrogenator. The mixture was degassed for 15 min with the help of alternative vacuum and nitrogen. The reaction was stirred under hydrogen atmosphere for 6 h at room temperature. The reaction was monitored by TLC (M.Ph: 5% methanol in DCM). The reaction mixture was filtered through Celite bed and the filtrate was concentrated in vacuo resulting in the crude compound. The crude compound was purified by trituration with diethyl ether and n-hexane, filtered and dried under vacuum to afford 4 (400 mg, 83.1%) as brown solid. ¹H NMR (DMSO-d₆, 400 MHz): δ ppm 5.95-6.04 (m, 2H), 5.84-5.91 (m, 1H), 5.26 (br. s, 2H), 3.10-3.19 (m, 4H), 2.46-2.53 (m, 4H), 2.30 (s, 3H); LC-MS: m/z 209.90 [M+H]⁺.

### Step-3: N-(3-Fluoro-5-(4-Methylpiperazin-1-yl)Phenyl)-4-Hydroxy-1-Isobutyl-2-Oxo-1,2-Dihydroquinoline-3-Carboxamide (SSTN-583)

To a stirred mixture of ethyl 4-hydroxy-1-isobutyl-2-oxo-1,2-dihydroquinoline-3-carboxylate (300 mg, 1.036 mmol, 1 eq) in DMSO (5 mL) was added 3-fluoro-5-(4-methylpiperazin-1-yl)aniline (4) (260 mg, 1.244 mmol, 1.2 eq) at room temperature. The reaction mixture was further heated at 110° C. for 12 h. The progress of the reaction was monitored by TLC (M.Ph: 5% Methanol in DCM). The reaction mixture was poured into ice cold water (50 mL) and extracted with ethyl acetate (2 × 50 mL). The combined organic layer was washed with ice cold water (2 × 50 mL) followed by brine (2 x 50 mL). The organic layer was separated, dried over anhydrous sodium sulfate, filtered and concentrated in vacuo resulting in the crude compound. The crude compound was purified through 230-400 mesh size silica gel column chromatography (elution: 0-5% methanol in DCM) to afford SSTN-583 (80 mg, 17.6%) as an off white solid. ¹H NMR (CDCl₃, 400 MHz): δ ppm 16.46 (s, 1H), 12.69 (br. s, 1H), 8.27 (d, J=7.82 Hz, 1H), 7.70 (t, J=7.82 Hz, 1H), 7.38 (d, J=8.80 Hz, 1H), 7.32 (t, J=7.58 Hz, 1H), 7.14 (d, J=10.27 Hz, 1H), 7.00 (br. s, 1H), 6.40 (d, J=11.25 Hz, 1H), 4.15-4.23 (m, 2H), 3.46-3.53 (m, 4H), 2.95-3.05 (m, 4H), 2.67 (s, 3H), 2.20-2.30 (m, 1H), 1.02 (d, J=6.85 Hz, 6H); LC-MS: m/z 453.10 [M+H]⁺; HPLC: 95.40%

Synthesis ofN-(3-fluoro-5-(4-methylpiperazin-1-yl)phenyl)-4-hydroxy-1-isobutyl-2-oxo-1,2-dihydroquinoline-3-carboxamide hydrochloride salt (SSTN-583_HCl salt, Notch1 Reporter Assay IC₅₀: 6.33 µM)

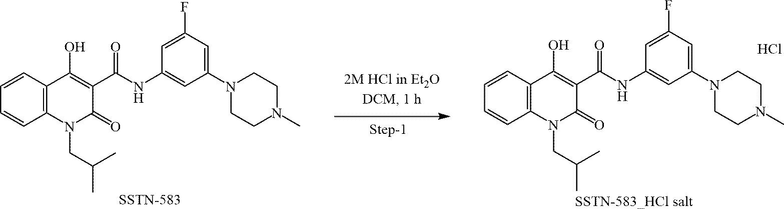

### Step-1: Synthesis of N-(3-Fluoro-5-(4-Methylpiperazin-1-yl)Phenyl)-4-Hydroxy-1-Isobutyl-2-Oxo-1,2-Dihydroquinoline-3-Carboxamide Hydrochloride Salt (SSTN-583_HCl Salt)

To a stirred mixture of N-(3-fluoro-5-(4-methylpiperazin-1-yl)phenyl)-4-hydroxy-1-isobutyl-2-oxo-1,2-dihydroquinoline-3-carboxamide (SSTN-583) (20 mg, 0.044 mmol, 1 eq) in Et₂O (1 mL) at was added 2 M HCl in dioxane (100 µL). The reaction mixture was stirred for 1 h then concentrated in vacuo resulting in the crude compound. The crude compound was purified by trituration with Et₂O/DCM, filtered and dried under vacuum to afford SSTN-583_HCl salt (19.1 mg, 89%) an off-white solid. ¹H NMR (400 MHz, DMSO) δ 10.41 (s, 1H), 8.16 (dd, J= 8.1, 1.6 Hz, 1H), 7.84 (ddd, J= 8.7, 7.0, 1.7 Hz, 1H), 7.74 (d, J= 8.7 Hz, 1H), 7.41 (t, J= 7.6 Hz, 1H), 7.24 (dt, J= 10.6, 2.0 Hz, 1H), 6.93 (t, J= 2.1 Hz, 1H), 6.73 (dt, J= 12.2, 2.3 Hz, 1H), 4.23 (dd, J= 10.8, 7.2 Hz, 2H), 3.94 (d, J= 9.4 Hz, 2H), 3.55 -3.44 (m, 3H), 3.11 (d, J= 8.1 Hz, 4H), 2.82 (d, J= 4.0 Hz, 3H), 2.17 (dq, J= 13.5, 6.9 Hz, 1H), 0.92 (d, J= 6.6 Hz, 6H). LC-MS: m/z 453.1 [M+H]⁺.

SSTN-584 (formate salt, HCl salt)

Synthesis ofN-(4-fluoro-3-(4-methylpiperazin-1-yl)phenyl)-4-hydroxy-1-isobutyl-2-oxo-1,2-dihydroquinoline-3-carboxamide formate salt (SSTN-584_Formate salt, Notch1 Reporter Assay IC₅₀: 4.54 µM)

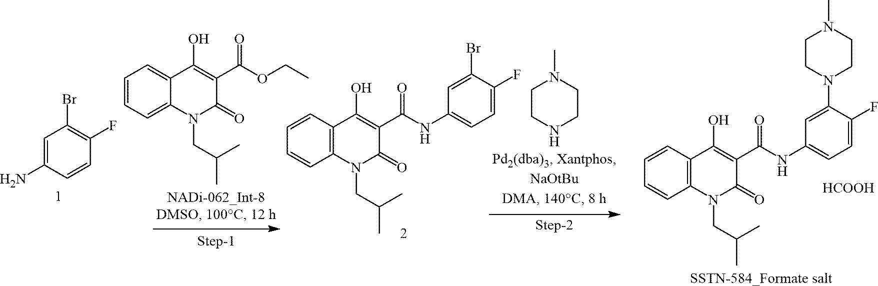

### Step-1: N-(3-Bromo-4-Fluorophenyl)-4-Hydroxy-1-Isobutyl-2-Oxo-1,2-Dihydroquinoline-3-Carboxamide (2)

To a stirred mixture of ethyl 4-hydroxy-1-isobutyl-2-oxo-1,2-dihydroquinoline-3-carboxylate (1.00 g, 3.456 mmol, 1 eq) in DMSO (10 mL) was added 3-bromo-4-fluoroaniline (1) (780 mg, 4.147 mmol, 1.2 eq) at room temperature. The reaction mixture was further heated at 100° C. for 12 h. The progress of the reaction was monitored by TLC (M.Ph: 30% ethyl acetate in n-hexane). The reaction mixture was poured into ice cold water (50 mL) and extracted with EtOAc (250 mL). The organic layer was washed with ice cold water (2 × 250 mL) followed by brine (2 × 250 mL). The organic layer was separated, dried over anhydrous sodium sulfate, filtered and concentrated in vacuo resulting in the crude compound. The crude compound was purified through 100-200 mesh size silica gel column chromatography (elution: 0-5% ethyl acetate in n-hexane) to afford 2 (620 mg, 44.2%) as a white solid. ¹H NMR (DMSO-d₆, 400 MHz): δ ppm 16.28 (br. s, 1H), 12.74 (br. s, 1H), 8.14-8.19 (m, 2H), 7.80-7.88 (m, 1H), 7.74 (d, J=8.80 Hz, 1H), 7.66 (dd, J=3.91, 8.80 Hz, 1H), 7.40-7.47 (m, 2H), 4.22 (d, J=6.85 Hz, 2H), 2.13-2.22 (m, 1H), 0.94 (d, J=6.36 Hz, 6H).

### Step-2: N-(4-Fluoro-3-(4-Methylpiperazin-1-yl)Phenyl)-4-Hydroxy-1-Isobutyl-2-Oxo-1,2-Dihydroquinoline-3-Carboxamide Formate Salt(SSTN-584_Formate Salt)

To a stirred mixture of N-(3-bromo-4-fluorophenyl)-4-hydroxy-1-isobutyl-2-oxo-1,2-dihydroquinoline-3-carboxamide (2) (270 mg, 0.623 mmol, 1 eq) and 1-methylpiperazine (74 mg, 0.747 mmol, 1.2 eq) in DMA (10 mL) was added NaOtBu (119 mg, 1.246 mmol, 2 eq) and degassed with argon for 10-15 min at room temperature. To the resulting solution was added Pd₂(dba)₃ (57 mg, 0.062 mmol, 0.1 eq), Xphos (59 mg, 0.124 mmol, 0.2 eq) and degassed with argon for another 10-15 min. The reaction mixture was further heated at 190° C. for 8 h. The progress of the reaction was monitored by TLC (M.Ph: 8% Methanol in DCM). The reaction mixture was filtered through a Celite bed and the filtrate was diluted with ethyl acetate (100 mL). The organic layer was separated from the filtrate and washed with water (2 × 100 mL) followed by brine (100 mL). The organic layer was dried over anhydrous sodium sulfate, filtered and concentrated in vacuo resulting in the crude compound. The crude compound was purified through 230-400 mesh size silica gel column chromatography (elution: 0-8% methanol in DCM). Compound obtained after column purification was repurified by preparative HPLC to afford SSTN-584_Formate salt (20 mg, 6.45%) as an off white solid. ¹H NMR (DMSO-d₆, 400 MHz): δ ppm 12.62 (br. s, 1H), 8.16 (d, J=8.31 Hz, 1H), 8.14 (s, 1H), 7.80-7.88 (m, 1H), 7.73 (d, J=8.80 Hz, 1H), 7.41 (t, J=7.58 Hz, 1H), 7.25-7.34 (m, 2H), 7.16 (dd, J=8.80, 12.23 Hz, 1H), 4.22 (d, J=5.87 Hz, 2H), 3.02-3.10 (m, 4H), 2.52-2.56 (m, 4H), 2.26 (s, 3H), 2.18 (td, J=6.66, 13.57 Hz, 1H), 0.93 (d, J=6.36 Hz, 6H); LC-MS: m/z 453.10 [M+H]⁺; HPLC: 99.01%.

Synthesis ofN-(4-fluoro-3-(4-methylpiperazin-1-yl)phenyl)-4-hydroxy-1-isobutyl-2-oxo-1,2-dihydroquinoline-3-carboxamide hydrochloride salt (SSTN-584_HCl salt, Notch1 Reporter Assay IC₅₀: 6.04 µM)

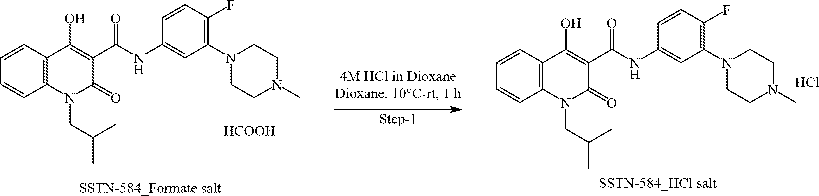

### Step-1: N-(4-Fluoro-3-(4-Methylpiperazin-1-yl)Phenyl)-4-Hydroxy-1-Isobutyl-2-Oxo-1,2-Dihydroquinoline-3-Carboxamide Hydrochloride Salt (SSTN-584_HCl Salt)

To a stirred mixture of N-(4-fluoro-3-(4-methylpiperazin-1-yl)phenyl)-4-hydroxy-1-isobutyl-2-oxo-1,2-dihydroquinoline-3-carboxamide formate salt (SSTN-584_Formate salt) (10 mg, 0.020 mmol, 1 eq) in dioxane (1 mL) at 10° C. was added 4 M HCl in dioxane (0.2 mL). The reaction mixture was allowed to attain room temperature and stirred for 1 h. The progress of the reaction was monitored by TLC (M.Ph: 5% Methanol in DCM). The reaction mixture was concentrated in vacuo resulting in the crude compound. The crude compound was purified by trituration with n-hexane (5 mL), filtered and dried under vacuum to afford SSTN-584_HCl salt (8.2 mg, 83.6%) as an off white solid. ¹H NMR (DMSO-d₆, 400 MHz): δ ppm 16.56 (s, 1H), 12.63 (br. s, 1H), 10.38 (br. s, 1H), 8.16 (d, J=7.82 Hz, 1H), 7.84 (t, J=7.09 Hz, 1H), 7.74 (d, J=8.31 Hz, 1H), 7.36-7.47 (m, 3H), 7.23 (dd, J=8.56, 11.98 Hz, 1H), 4.19-4.27 (m, 2H), 3.48-3.64 (m, 4H), 3.08-3.29 (m, 4H), 2.85 (br. s, 3H), 2.19 (dd, J=6.60, 13.45 Hz, 1H), 0.93 (d, J=6.36 Hz, 6H); LC-MS: m/z 453.10 [M+H]⁺; HPLC: 96.52%.

SSTN-586

Synthesis of tert-butyl 4-(3-((3-fluorophenyl)carbamoyl)-4-hydroxy-1-isobutyl-2-oxo-1,2-dihydroquinolin-5-yl)piperazine-1-carboxylate (SSTN-586)

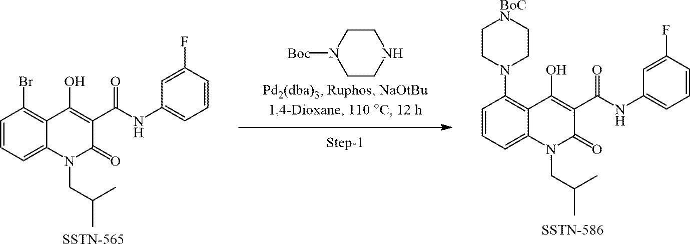

### Step-1: Tert-Butyl 4-(3-((3-Fluorophenyl)Carbamoyl)-4-Hydroxy-1-Isobutyl-2-Oxo-1,2-Dihydroquinolin-5-yl)Piperazine-1-Carboxylate (SSTN-586)

To a stirred mixture of 5-bromo-N-(3-fluorophenyl)-4-hydroxy-1-isobutyl-2-oxo-1,2-dihydroquinoline-3-carboxamide (SSTN-565) (400 mg, 0.923 mmol, 1 eq) and tert-butyl piperazine-1-carboxylate (189 mg, 1.015 mmol, 1.1 eq) in 1,4-dioxane (20 mL) was added NaOtBu (177 mg, 1.846 mmol, 2 eq) and degassed with argon for 10 min at room temperature. To the resulting solution was added Ruphos (64 mg, 0.138 mmol, 0.15 eq), catalyst Pd(OAc)₂ (20 mg, 0.092 mmol, 0.1 eq) and degassed with argon for another 10 min. The reaction mixture was further heated at 110° C. for 12 h in a sealed tube. The progress of the reaction was monitored by TLC (M.Ph: 80% EtOAc in n-hexane). The reaction mixture was cooled to room temperature, diluted with EtOAc (100 mL) and filtered through a Celite bed. The filtrate was washed with water (2 × 50 mL) followed by brine (2 × 50 mL). The organic layer was separated, dried over anhydrous sodium sulfate, filtered and concentrated in vacuo resulting in the crude compound. The crude compound was purified through 230-400 mesh size silica gel column chromatography (elution: 10-90% EtOAc in n-hexane). Compound obtained after column purification was repurified by preparative HPLC to afford SSTN-586 (53 mg, 10.6%) as yellow solid. ¹H NMR (DMSO-d₆, 400 MHz): δ ppm 16.90 (br. s, 1H), 11.53 (br. s, 1H), 7.63-7.75 (m, 2H), 7.36-7.45 (m, 3H), 7.29 (d, J=7.82 Hz, 1H), 6.94 (br. s, 1H), 3.98-4.19 (m, 4H), 3.01-3.21 (m, 4H), 2.90-2.96 (m, 2H), 2.08-2.16 (m, 1H), 1.43 (s, 9H), 0.91 (d, J=6.36 Hz, 6H); LC-MS: m/z 539.30 [M+H]⁺; HPLC: 99.76%.

SSTN-587

N-(3-fluorophenyl)-4-hydroxy-1-isobutyl-2-oxo-5-(piperazin-1-yl)-1,2-dihydroquinoline-3-carboxamide hydrochloride salt (SSTN-587_HCl salt, Notch1 Reporter Assay IC₅₀: 3.76 µM)

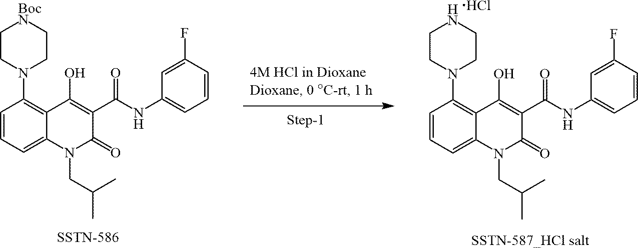

### Step-1: N-(3-Fluorophenyl)-4-Hydroxy-1-Isobutyl-2-Oxo-5-(Piperazin-1-yl)-1,2-Dihydroquinoline-3-Carboxamide Hydrochloride Salt (SSTN-587_HCl Salt)

To a stirred mixture of tert-butyl 4-(3-((3-fluorophenyl)carbamoyl)-4-hydroxy-1-isobutyl-2-oxo-1,2-dihydroquinolin-5-yl)piperazine-1-carboxylate (SSTN-586) (43 mg, 0.079 mmol, 1 eq) in 1,4-dioxane (2 mL) at 0° C. was added 4 M HCl in dioxane (0.5 mL). The reaction mixture was allowed to attain at room temperature and stirred for 1 h. The progress of the reaction was monitored by TLC (M.Ph: 80% ethyl acetate in n-hexane). The reaction mixture was concentrated in vacuo resulting in the crude compound. The crude compound was purified by trituration with n-hexane (5 mL), filtered and dried under vacuum to afford SSTN-587_HCl salt (35 mg, 92.3%) as an off-white solid. Crude compound obtained was used as such in the next step without further purification. ¹H NMR (DMSO-d₆, 400 MHz): δ ppm 16.96 (br. s, 1H), 12.66 (br. s, 1H), 8.93-9.14 (m, 2H), 7.71 (d, J=7.82 Hz, 2H), 7.32-7.47 (m, 3H), 6.91-7.10 (m, 2H), 4.14-4.28 (m, 2H), 3.32-3.40 (m, 3H), 3.05-3.30 (m, 5H), 2.08-2.22 (m, 1H), 0.92 (d, J=6.36 Hz, 6H); LC-MS: m/z 439.10 [M+H]⁺.

SSTN-588 (free base, HCl salt)

Synthesis ofN-(3-fluorophenyl)-4-hydroxy-1-isobutyl-5-(4-methylpiperazin-1-yl)-2-oxo-1,2-dihydroquinoline-3-carboxamide (SSTN-588, Notch1 Reporter Assay IC₅₀: 10.3 µM)

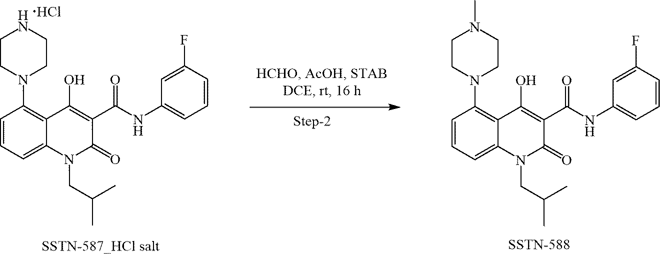

### Step-2: N-(3-Fluorophenyl)-4-Hydroxy-1-Isobutyl-5-(4-Methylpiperazin-1-yl)-2-Oxo-1,2-Dihydroquinoline-3-Carboxamide (SSTN-588)

To a stirred mixture of N-(3-fluorophenyl)-4-hydroxy-1-isobutyl-2-oxo-5-(piperazin-1-yl)-1,2-dihydroquinoline-3-carboxamide hydrochloride salt (SSTN-587_HCl salt) (15 mg, 0.031 mmol, 1 eq) in DCE (5 mL) was added formaldehyde (4.74 mg, 0.157 mmol, 5 eq) at room temperature. To the resulting solution was added STAB (26.7 g, 0.126 mmol, 4 eq) followed by acetic acid (0.009 mL, 0.157 mmol, 5 eq) and stirred for 5 min. The reaction mixture was allowed to attain room temperature and stirred for 16 h. The reaction was monitored by TLC (M.Ph: 5% methanol in DCM). The reaction mixture was concentrated in vacuo upto dryness resulting in the crude compound. The crude compound was purified through 100-200 mesh size silica gel column chromatography (elution: 0-5% methanol in DCM). Compound obtained after column chromatography purification was repurified by preparative TLC to afford SSTN-588 (9 mg, 37.8%) as yellow solid. ¹H NMR (DMSO-d₆, 400 MHz): δ ppm 17.24 (br. s, 1H), 10.93 (br. s, 1H), 7.62-7.76 (m, 2H), 7.35-7.48 (m, 4H), 6.86-6.95 (m, 1H), 4.06-4.16 (m, 2H), 3.05-3.18 (m, 4H), 2.89 (d, J=9.29 Hz, 2H), 2.27 (br. s, 3H), 2.16-2.24 (m, 2H), 2.05-2.14 (m, 1H), 0.91 (d, J=6.36 Hz, 6H); LC-MS: m/z 453.10 [M+H]⁺; HPLC: 99.87%.

Synthesis ofN-(3-fluorophenyl)-4-hydroxy-1-isobutyl-5-(4-methylpiperazin-1-yl)-2-oxo-1,2-dihydroquinoline-3-carboxamide hydrochloride salt (SSTN-588_HCl salt, Notch1 Reporter Assay IC₅₀: 9.8 µM)

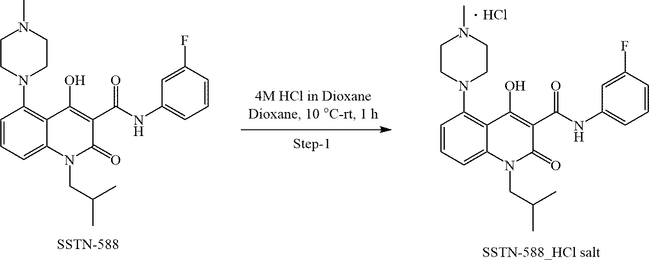 [SMILES: O=C(Nc1ncccc1O)c1c(O)c2c(n(Cc3ccco3)c1=O)CCCC2]

### Step-1: N-(3-Fluorophenyl)-4-Hydroxy-1-Isobutyl-5-(4-Methylpiperazin-1-yl)-2-Oxo-1,2-Dihydroquinoline-3-Carboxamide Hydrochloride Salt (SSTN-588_HCl Salt)

To a stirred mixture of N-(3-fluorophenyl)-4-hydroxy-1-isobutyl-5-(4-methylpiperazin-1-yl)-2-oxo-1,2-dihydroquinoline-3-carboxamide (SSTN-588) (4.5 mg, 0.0099 mmol, 1 eq) in dioxane (1 mL) at 10° C. was added 4 M HCl in dioxane (0.2 mL). The reaction mixture was allowed to attain at room temperature and stirred for 1 h. The progress of the reaction was monitored by TLC (M.Ph: 5% Methanol in DCM). The reaction mixture was concentrated in vacuo resulting in the crude compound. The crude compound was purified by trituration with n-hexane (5 mL), filtered and dried under vacuum to afford SSTN-588_HCl salt (4.6 mg, 95.8%) as yellow solid. ¹H NMR (DMSO-d₆, 400 MHz): δ ppm 16.96 (br. s, 1H), 12.69 (br. s, 1H), 10.35 (br. s, 1H), 7.69 (d, J=4.40 Hz, 2H), 7.34-7.49 (m, 3H), 7.03 (d, J=5.38 Hz, 2H), 4.21 (d, J=1.47 Hz, 2H), 3.46-3.60 (m, 6H), 3.15 (d, J=11.25 Hz, 2H), 2.88 (br. s, 3H), 2.10-2.19 (m, 1H), 0.92 (d, J=5.38 Hz, 6H); LC-MS: m/z 453.00 [M+H]⁺; HPLC: 97.67%.

TABLE 1

Additional compounds

| | | |
|---|---|---|
| 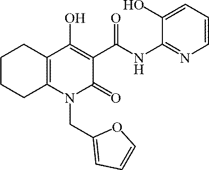 [SMILES: O=C(Nc1ccccn1)c1c(O)c2c(n(Cc3ccco3)c1=O)CCCC2] | 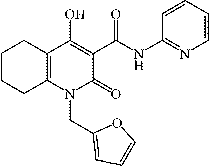 [SMILES: O=C(Nc1ccccc1)c1c(O)c2c(n(Cc3ccco3)c1=O)CCCC2] | 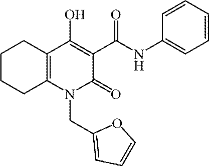 [SMILES: Cc1cccnc1NC(=O)c1c(O)c2c(n(Cc3ccco3)c1=O)CCCC2] |
| SSTN-293 SSTN-405 | SSTN-379 | SSTN-380 |
| 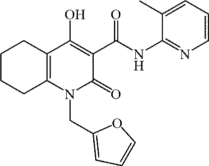 [SMILES: Cc1cccc(NC(=O)c2c(O)c3c(n(Cc4ccco4)c2=O)CCCC3)n1] | 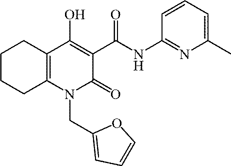 [SMILES: CCCn1c2c(c(O)c(C(=O)Nc3ncccc3O)c1=O)CCCC2] | 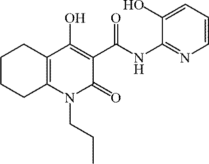 [SMILES: CCCn1c2c(c(O)c(C(=O)Nc3ccccn3)c1=O)CCCC2] |
| SSTN-403 | SSTN-404 | SSTN-401 |
| 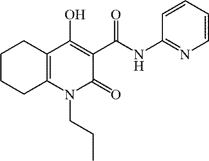 [SMILES: CC(C)Cn1c2c(c(O)c(C(=O)Nc3ncccc3O)c1=O)CCCC2] | 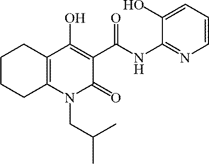 [SMILES: O=C(Nc1ncccc1O)c1c(O)c2c([nH]c1=O)CCCC2] | 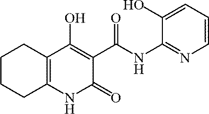 [SMILES: Cc1cccnc1NC(=O)c1c(O)c2c([nH]c1=O)CCCC2] |
| SSTN-400 | SSTN-402 | SSTN-407 |
| 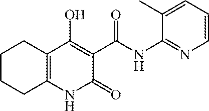 [SMILES: O=C(Nc1ncccc1O)c1c(O)c2ccccc2[nH]c1=O] | 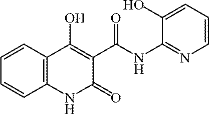 [SMILES: Cn1c(=O)c(C(=O)Nc2ncccc2O)c(O)c2ccccc21] | 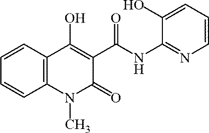 [SMILES: O=C(Nc1ncccc1O)c1c(O)c2ccccc2n(Cc2ccccc2)c1=O] |
| SSTN-406 | SSTN-397 | SSTN-398 |
| 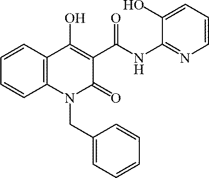 [SMILES: O=C(Nc1ccccn1)c1c(O)c2ccccc2n(Cc2ccccc2)c1=O] | 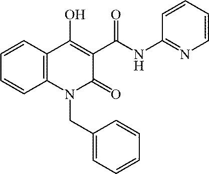 [SMILES: Cc1cccnc1NC(=O)c1c(O)c2ccccc2n(Cc2ccccc2)c1=O] | 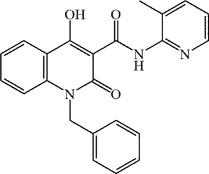 [SMILES: Cc1ccnc(NC(=O)c2c(O)c3ccccc3n(Cc3ccccc3)c2=O)c1] |
| SSTN-409 | SSTN-408 SSTN-452 | SSTN-410 SSTN-455 |
| 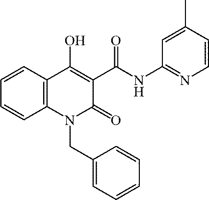 [SMILES: Cc1ccc(NC(=O)c2c(O)c3ccccc3n(Cc3ccccc3)c2=O)nc1] | 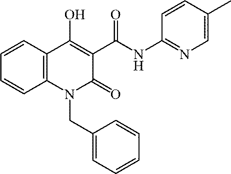 [SMILES: O=C(Nc1cccnc1)c1c(O)c2ccccc2n(-c2ccccc2)c1=O] | 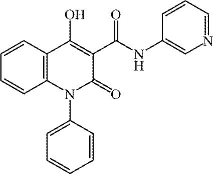 [SMILES: O=C(Nc1ccncc1)c1c(O)c2ccccc2n(-c2ccccc2)c1=O] |
| SSTN-411 | SSTN-412 | SSTN-441 |
| 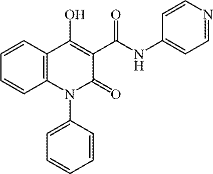 [SMILES: O=C(NCc1ccccc1)c1c(O)c2ccccc2n(-c2ccccc2)c1=O] | 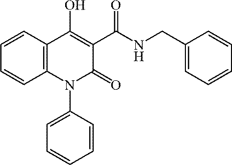 [SMILES: O=C(Nc1ccc(F)cc1)c1c(O)c2cccnc2n(-c2ccccc2)c1=O] | 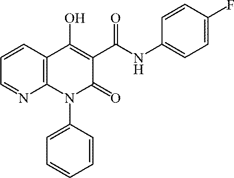 |
| SSTN-442 | SSTN-443 | SSTN-444 |
| 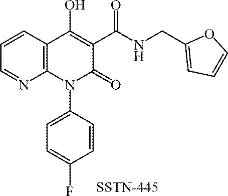 [SMILES: O=C(NCC1CC1)c1c(O)c2cccnc2n(-c2cccc(F)c2)c1=O] | 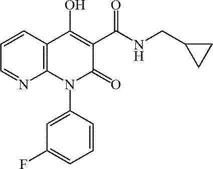 [SMILES: CCn1c(=O)c(C(=O)Nc2ccccn2)c(O)c2ccccc21] | 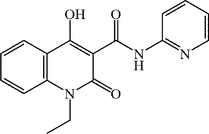 |
| | SSTN-446 | SSTN-449 |
| 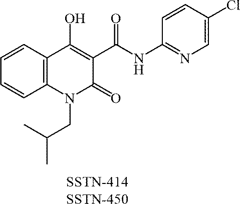 [SMILES: CCn1c(=O)c(C(=O)Nc2ccc(Cl)cn2)c(O)c2ccccc21] | 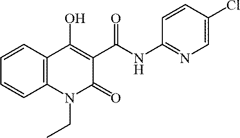 [SMILES: Cc1ccnc(NC(=O)c2c(O)c3ccccc3n(Cc3ccccc3)c2=O)c1] | 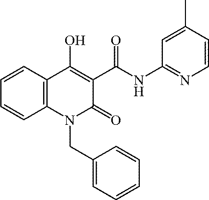 [SMILES: Cc1ccc(NC(=O)c2c(O)c3ccccc3n(Cc3ccccc3)c2=O)nc1] |
| | SSTN-451 | SSTN-453 |
| 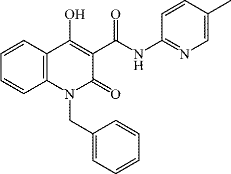 [SMILES: Cc1cccnc1NC(=O)c1c(O)c2ccccc2n(Cc2ccccc2)c1=O] | 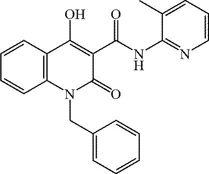 [SMILES: Cc1cccc(NC(=O)c2c(O)c3ccccc3n(Cc3ccccc3)c2=O)n1] | 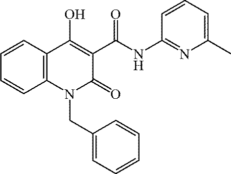 [SMILES: CC(C)Cn1c2c(c(O)c(C(=O)Nc3ncccc3O)c1=O)CCCC2] |
| SSTN-454 | SSTN-455 | SSTN-456 |
| 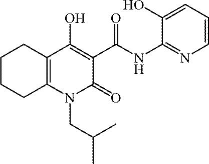 [SMILES: CCCn1c2c(c(O)c(C(=O)Nc3ccccn3)c1=O)CCCC2] | 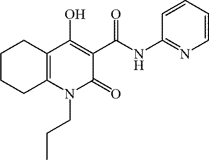 [SMILES: Cc1cccnc1NC(=O)c1c(O)c2cc(Cl)ccc2n(CC(C)C)c1=O] | 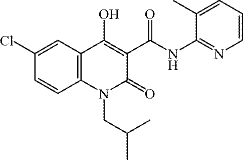 [SMILES: Cc1cccnc1NC(=O)c1c(O)c2cc(C(F)(F)F)ccc2n(CC(C)C)c1=O] |
| SSTN-457 | SSTN-400 | SSTN-515 |
| 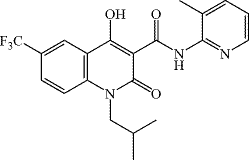 [SMILES: CC(C)Cn1c(=O)c(C(=O)Nc2cnccc2O)c(O)c2ccccc21] | 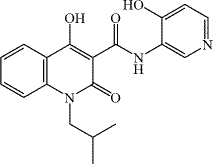 [SMILES: CC(=O)Nc1ccccc1NC(=O)c1c(O)c2ccccc2n(CC(C)C)c1=O] | 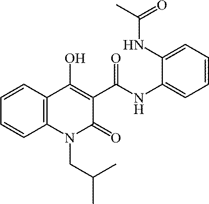 [SMILES: CC(C)Cn1c(=O)c(C(=O)Nc2ccncc2O)c(O)c2ccccc21] |
| SSTN-516 | SSTN-521 | SSTN-523 |
| 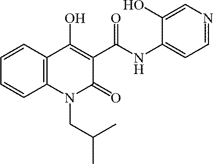 [SMILES: CC(C)Cn1c(=O)c(C(=O)Nc2ncc(Cl)cc2O)c(O)c2ccccc21] | 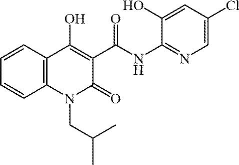 [SMILES: CC(C)Cn1c(=O)c(C(=O)Nc2ncccc2OCc2ccccc2)c(O)c2ccccc21] | 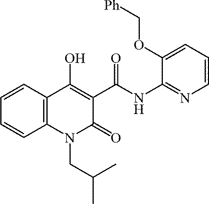 [SMILES: COc1cccnc1NC(=O)c1c(O)c2ccccc2n(CC(C)C)c1=O] |
| SSTN-524 | SSTN-529 | SSTN-530 |
| 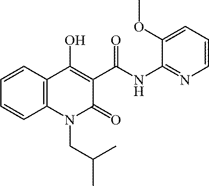 [SMILES: COc1ccccc1NC(=O)c1c(O)c2ccccc2n(CC(C)C)c1=O] | 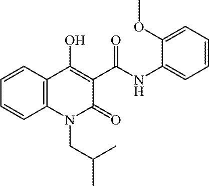 [SMILES: CC(C)Cn1c(=O)c(C(=O)Nc2cccc(F)c2)c(O)c2ccc(N3CCN(C)CC3)cc21 ; Cl] | 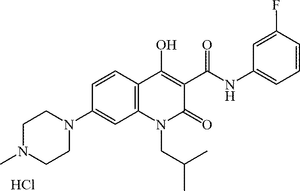 |
| SSTN-531 | SSTN-536 | SSTN-585 |

### Example 2: Biological Assay Procedures and Activity Data

### Notch1-3 Reporter Assay

293A cells expressing 1) pCMV-Tet-On 3G, 2) pLV[Tet]-Puro-TRE3G>Notch1-ICD, 3) pLV[Tet]-Puro-TRE3G>Notch2-ICD, 4) pLV[Tet]-Puro-TRE3G>Notch3-ICD, and 5) pCSL-RElement-Luc were used. When Doxycycline is added, the Tet-ON gene activates expression of hNotch1ICD, which together with endogenous NTC components binds to CSL responsive elements (pCSL-RElement-Luc) and expresses luciferase. 1 ×× 10⁴ cells are plated in 100 µL (96-well format). 24 h later, compounds (10 mM stock in DMSO) are diluted in DMSO to 200X, then added (5 ul into 1 mL) to cell culture media containing 50 ng/mL Dox. This is then added 1:1 to cells. Final DMSO = 0.25%, Dox = 25 ng/mL. After 24 h, media is discarded, and cells are lysed in passive lysis buffer (Promega). Cells are rocked at room temperature for 15 min and then lysate is divided for luciferase assay (Luciferase Assay System, Promega) and cell viability (CellTiter Glo 2.0, Promega). Raw luciferase is normalized to cell viability, and then scaled to DMSO wells. Results are analyzed and IC₅₀′s are determined in GraphPad by nonlinear regression curve fitting (4 parameter) of dose response curves.

### OE33 Colony Formation Assay

The OE33 Esophageal Adenocarcinoma cell line was cultured and plated into 96-well tissue culture plates under sparse conditions (200 cells/well). Test compounds were serially diluted in DMSO and then into culture media (final DMSO concentration = 0.1%). Compound/media was then added to cells every 48 hours for a total of 7 days. Clonogenic growth was assessed using the CellTiter-Glo reagent, according to manufacturer (Promega) specifications. Percent inhibition was calculated as the percent of luminescence normalized to control (0.1% DMSO) wells. Nonlinear regression curve fitting was performed using GraphPad Prism software to determine EC₅₀′s.

TABLE 2

Biological data

| Compound (SSTN-XXX) | Notch1 Reporter Assay IC₅₀, µM | OE33 Colony Formation Assay EC₅₀, µM |
|---|---|---|
| 293 | 12.8 | 3.4 |
| 379 | 27.5 | N.T. |
| 380 | n/a | N.T. |
| 397 | n/a | N.T. |
| 398 | n/a | N.T. |
| 400 | n/a | N.T. |
| 401 | 21.6 | N.T. |
| 402 | 12.1 | N.T. |
| 403 | n/a | N.T. |
| 404 | n/a | N.T. |
| 406 | n/a | N.T. |
| 407 | n/a | N.T. |
| 408 | 5 | 3.7 |
| 409 | 5.8 | N.T. |
| 410 | 21.9 | N.T. |
| 411 | n/a | N.T. |
| 412 | n/a | N.T. |
| 414 | 11.8 | N.T. |
| 441 | n/a | N.T. |
| 442 | 22.8 | n/a |
| 443 | 9 | n/a |
| 444 | n/a | N.T. |
| 445 | 13.7 | 5.9 |
| 446 | n/a | N.T. |
| 449 | n/a | N.T. |
| 450 | 8.5 | 2.1 |
| 451 | n/a | N.T. |
| 452 | 5.4 | 1.6 |
| 453 | n/a | N.T. |
| 454 | n/a | N.T. |
| 455 | 22.7 | N.T. |
| 456 | n/a | N.T. |
| 457 | 17.6 | N.T. |
| 513 | 11.1 | N.T. |
| 514 | 13.6 | N.T. |
| 515 | n/a | N.T. |
| 516 | n/a | N.T. |
| 517 | 22.6 | n/a |
| 518 | 18.7 | n/a |
| 519 | 17.8 | n/a |
| 521 | n/a | N.T. |
| 522 | 17.2 | 6 |
| 523 | n/a | N.T. |
| 524 | n/a | N.T. |
| 525 | 13.8 | 7.4 |
| 526 | 20.3 | N.T. |
| 527 | 8.5 | N.T. |
| 528 | 14.3 | N.T. |
| 529 | n/a | N.T. |
| 530 | n/a | N.T. |
| 531 | n/a | N.T. |
| 532 | 12.4 | N.T. |
| 533 | 22.5 | N.T. |
| 534 | 11.3 | 2.5 |
| 535 | 5.9 | N.T. |
| 536 | n/a | N.T. |
| 537 | 5.1 | N.T. |
| 538 | 9.6 | 1.7 |
| 539 | 6.6 | N.T. |
| 540 | 18.5 | N.T. |
| 541 | 15.1 | N.T. |
| 549 | 10.1 | N.T. |
| 550 | 20.8 | N.T. |
| 551 | 6.7 | N.T. |
| 552 | 4.3 | 0.9 |
| 553 | n/a | N.T. |
| 554 | 4.3 | N.T. |
| 560(01) | 12.3 | 2.3 |
| 560(02) | 7.7 | 4.1 |
| 560(03) | 10.2 | N.T. |
| 560(04) | 9 | 2.3 |
| 561 | 19.1 | 3.9 |
| 562 | 15 | 7.7 |
| 563 | 8 | N.T. |
| 564 | n/a | N.T. |
| 565 | n/a | N.T. |
| 566 | n/a | N.T. |
| 567 | 33.2 | N.T. |
| 568(01) | n/a | N.T. |
| 568(02) | 6.8 | 2.6 |
| 569(01) | 8.4 | N.T. |
| 569(02) | 4.9 | 1.8 |
| 570(01) | 6.5 | N.T. |
| 570(02) | 5.6 | 2 |
| 571(01) | n/a | N.T. |
| 571(02) | 7.9 | 2.5 |
| 572(01) | 17.7 | N.T. |
| 572(02) | 3.4 | 2.2 |
| 576 | n/a | N.T. |
| 577(01) | 9.7 | N.T. |
| 577(02) | 6.4 | 0.66 |
| 578(01) | n/a | N.T. |
| 578(02) | n/a | N.T. |
| 579(01) | 18.4 | N.T. |
| 579(02) | n/a | N.T. |
| 580(01) | 13.3 | N.T. |
| 580(02) | 14 | N.T. |
| 581(01) | 7.8 | N.T. |
| 581(02) | 7.7 | N.T. |
| 582(01) | n/a | N.T. |
| 582(02) | 1.9 | 1.1 |
| 583(01) | 8.1 | N.T. |
| 583(02) | 6.3 | N.T. |
| 584(01) | 4.5 | N.T. |
| 584(02) | 6 | 2 |
| 585 | n/a | N.T. |
| 586 | n/a | N.T. |
| 587 | 3.8 | 1.7 |
| 588(01) | 10.3 | N.T. |
| 588(02) | 9.8 | N.T. |

N.T. = not tested.

What is claimed is:

1. A compound having the formula: 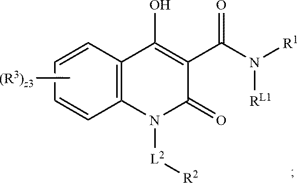
wherein 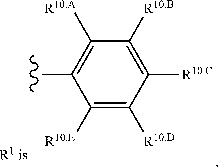 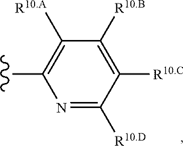 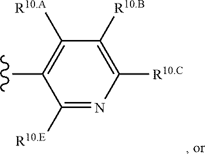 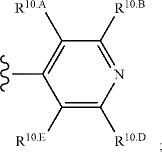
R^{10.A} is hydrogen, halogen, —CX^{10.A}₃, —CHX^{10.A}₂, —CH₂X¹⁰^{.A}, —OCX^{10.A}₃, —OCH₂X^{10.A}, —OCHX^{10.A}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10.B} is hydrogen, halogen, —CX^{10.B}₃, —CHX^{10.B}₂, —CH₂X^{10.B}, —OCX^{10.B}₃, —OCH₂X^{10.B}, —OCHX^{10.B}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10.C} is hydrogen, halogen, —CX^{10.C}₃, —CHX^{10.C}₂, —CH₂X^{10.C}, —OCX^{10.C}₃, —OCH₂X^{10.C}, —OCHX^{10.c}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10.D} is hydrogen, halogen, —CX^{10.D}₃, —CHX^{10.D}₂, —CH₂X^{10.D}, —OCX^{10.D}₃, —OCH₂X^{10D}, —OCHX^{10.D}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{10.E} is hydrogen, halogen, —CX^{10.E}₃, —CHX^{10.E}₂, —CH₂X^{10.E}, —OCX^{10.E}₃, —OCH₂X^{10.E}, —OCHX^{10.E}₂, —CN, —SOₙ₁₀R^{10D}, —SOᵥ₁₀NR^{10A}R^{10B}, —NR^{10C}NR^{10A}R^{10B}, —ONR^{10A}R^{10B}, —NHC(O)NR^{10C}NR^{10A}R^{10B}, —NHC(O)NR^{10A}R^{10B}, —N(O)ₘ₁₀, —NR^{10A}R^{10B}, —C(O)R^{10C}, —C(O)—OR^{10C}, —C(O)NR^{10A}R^{10B}, —OR^{10D}, —NR^{10A}SO₂R^{10D}, —NR^{10A}C(O)R^{10C}, —NR^{10A}C(O)OR^{10C}, —NR^{10A}OR^{10C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, -OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R³ is independently halogen, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 4;
Ra^{2A}, R^{2B}, R^{2C}, R^{2D}, R^{3A}, R^{3B}, R^{3C}, R^{3D}, R^{10A}, R^{10B}, R^{10C}, R^{10D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHl₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{10A} and R^{10B} substituents bonded to the same nitrogen atom may optionally be joined to form a substituted or unsubstituted heterocycloalkyl or substituted or unsubstituted heteroaryl;
X², X³, X^{10.A}, X^{10.B}, X^{10.C}, x^{10.D}, and X^{10.E} are independently —F, —Cl, —Br, or —I;
n2, n3, and n10 are independently an integer from 0 to 4; and
m2, m3, m10, v2, v3, and v10 are independently 1 or 2;
or a pharmaceutically acceptable salt thereof;
wherein —L²—R² is not hydrogen; and
wherein at least one of R^{10.A}, R^{10.B}, R^{10.C} , R^{10.D}, or R^{10.E} is a substituted or unsubstituted cycloalkyl or substituted or unsubstituted heterocycloalkyl.

2. The compound of claim 1, wherein R¹ is 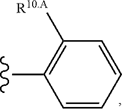 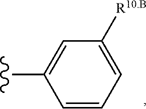 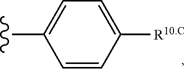 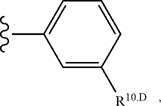 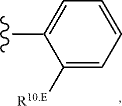 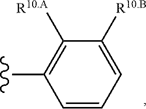 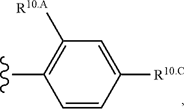 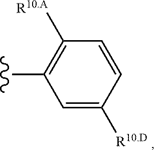 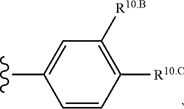 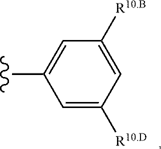 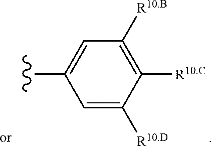
.

3. The compound of claim 1, wherein R^{10.A}, R^{10.B}, R^{10.C}, R^{10.D}, or R^{10.E} is independently halogen, substituted or unsubstituted C₆ cycloalkyl, or substituted or unsubstituted 6 membered heterocycloalkyl.

4. The compound of claim 1, wherein R^{10.A}, R^{10.B}, R^{10.C}, R^{10.D}, or R^{10.E} is independently a substituted or unsubstituted 6 membered heterocycloalkyl.

5. The compound of claim 1, wherein R^{10.A}, R^{10.B}, R^{10.C}, R^{10.D}, or R^{10.E} is independently a substituted or unsubstituted morpholinyl or substituted or unsubstituted piperazinyl.

6. The compound of claim 1, wherein R^{10.A}, R^{10.B}, R^{10.C}, R^{10.D}, or R^{10.E} is independently 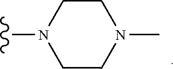
.

7. The compound of claim 1, wherein R¹ is 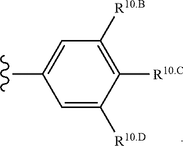
.

8. The compound of claim 7, wherein R^{10.B} and R^{10.D} are independently halogen, and R^{10.C} is substituted or unsubstituted C₆ cycloalkyl or substituted or unsubstituted 6 membered heterocycloalkyl.

9. The compound of claim 1, wherein R¹ is 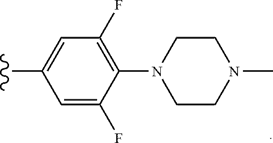 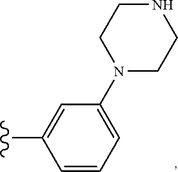 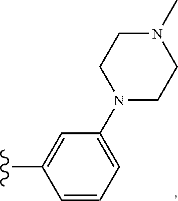 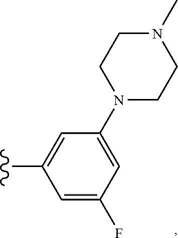 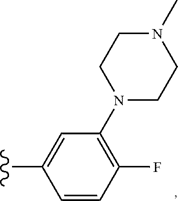 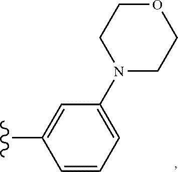 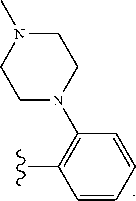 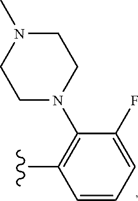 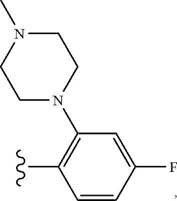 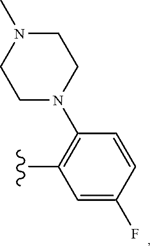 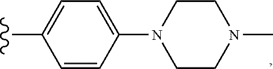 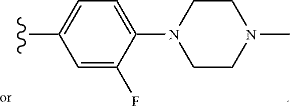
.

10. The compound of claim 1, wherein R^{L1} is hydrogen, unsubstituted methyl, unsubstituted ethyl, unsubstituted isopropyl, or unsubstituted cyclopropyl.

11. The compound of claim 1, wherein R^{L1} is hydrogen.

12. The compound of claim 1, wherein z3 is 0.

13. The compound of claim 1, wherein R³ is independently halogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —NO₂, —SH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —CH₃, —CH₂CH₃, —OCH₃, —OCH₂CH₃, or substituted or unsubstituted 3 to 6 membered heterocycloalkyl.

14. The compound of claim 1, wherein R³ is independently substituted or unsubstituted 3 to 6 membered heterocycloalkyl.

15. The compound of claim 1, wherein R³ is independently substituted or unsubstituted morpholinyl or substituted or unsubstituted piperazinyl.

16. The compound of claim 1, wherein R³ is independently —Br, —OCH₃, or substituted or unsubstituted piperazinyl.

17. The compound of claim 1, wherein L² is a bond or substituted or unsubstituted C₁—C₆ alkylene.

18. The compound of claim 1, wherein L² is a bond or unsubstituted C₁—C₄ alkylene.

19. The compound of claim 1, wherein L² is a bond.

20. The compound of claim 1, wherein L² is unsubstituted C₁—C₄ alkylene.

21. The compound of claim 1, wherein L² is unsubstituted methylene.

22. The compound of claim 1, wherein R² is hydrogen, halogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

23. The compound of claim 1, wherein R² is unsubstituted alkyl.

24. The compound of claim 1, wherein R² is unsubstituted C₁—C₄ alkyl.

25. The compound of claim 1, wherein R² is unsubstituted isobutyl.

26. The compound of claim 1, wherein R² is substituted or unsubstituted aryl or substituted or unsubstituted heteroaryl.

27. The compound of claim 1, wherein R² is substituted or unsubstituted phenyl or substituted or unsubstituted 5 to 6 membered heteroaryl.

28. The compound of claim 1, wherein
R2 is R²⁰-substituted phenyl or R²⁰-substituted 5 to 6 membered heteroaryl;
R²⁰ is independently halogen, —CX²⁰₃, —CHX²⁰₂, —CH₂X²⁰, —OCX²⁰₃, —OCH₂X²⁰, —OCHX²⁰₂, —CN, —SOₙ₂₀R^{20D}, —SOᵥ₂₀NR^{20A}R^{20B}, —NR^{20C}NR^{20A}R^{20B}, —ONR^{20A}R^{20B}, —NHC(O)NR^{20C}NR^{20A}R^{20B}, —NHC(O)NR^{20A}R^{20B}, —N(O)ₘ₂₀, —NR^{20A}R^{20B}, —C(O)R^{20C}, —C(O)—OR^{20C}, —C(O)NR^{20A}R^{20B}, —OR^{20D}, —NR^{20A}SO₂R^{20D}, —NR^{20A}C(O)R^{20C}, —NR^{20A}C(O)OR^{20C}, —NR^{20A}OR^{20C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R^{20A}, R^{20B}, R^{20C}, and R^{20D} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI₂, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{20A} and R^{20B} substituents bonded to the same nitrogen atom may optionally be joined to form a substituted or unsubstituted heterocycloalkyl or substituted or unsubstituted heteroaryl;
X²⁰ is independently —F, —Cl, —Br, or —I;
n20 is an integer from 0 to 4; and
m20 and v20 are independently 1 or 2.

29. The compound of claim 1, wherein
R2 is R²⁰-substituted phenyl or R²⁰-substituted 5 to 6 membered heteroaryl; and
R²⁰ is independently halogen.

30. The compound of claim 1, wherein
R2 is R²⁰-substituted phenyl or R²⁰-substituted 5 to 6 membered heteroaryl; and
R²⁰ is independently —F.

31. The compound of claim 1, wherein R² is unsubstituted phenyl or unsubstituted 5 to 6 membered heteroaryl.

32. A compound having the formula: 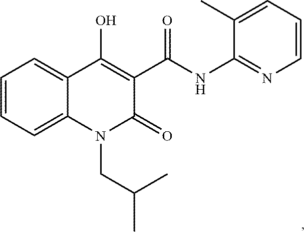 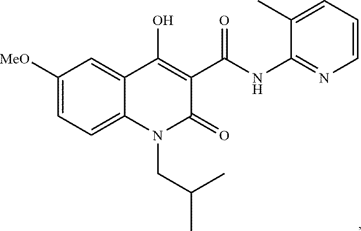 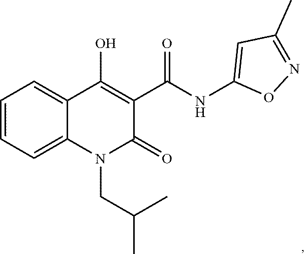 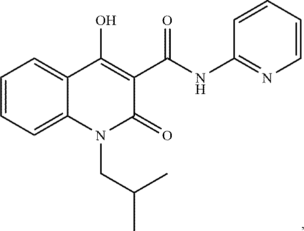 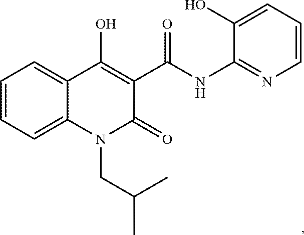 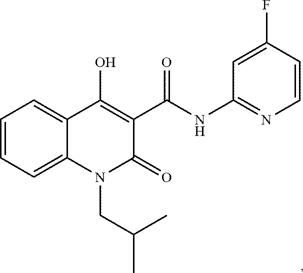 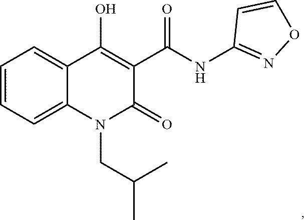 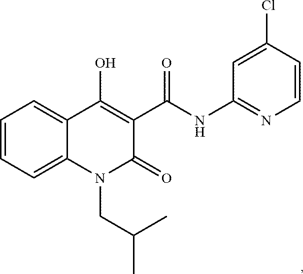 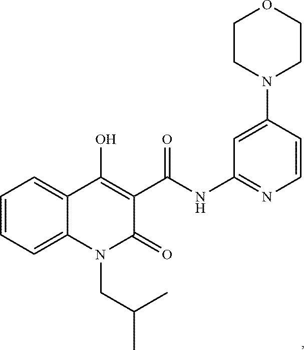 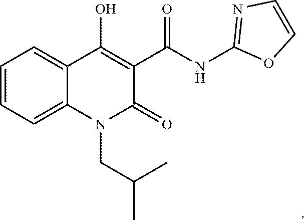 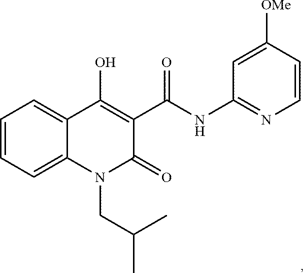 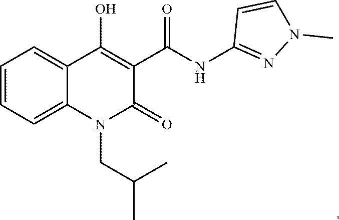 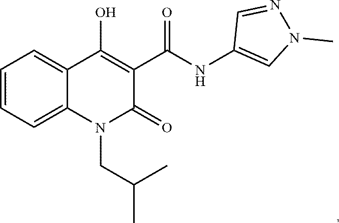 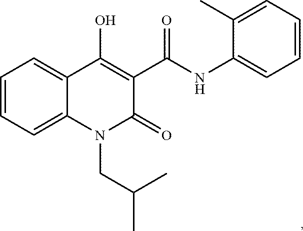 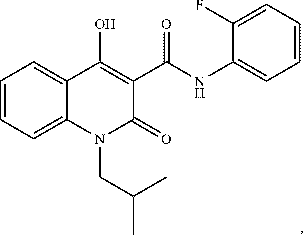 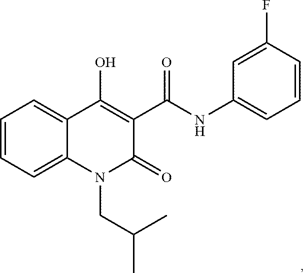 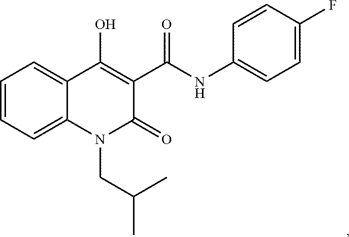 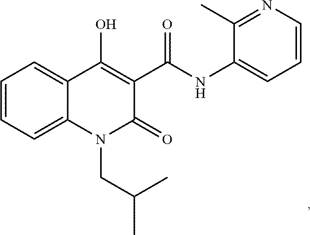 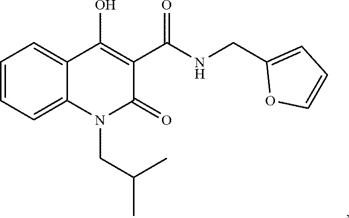 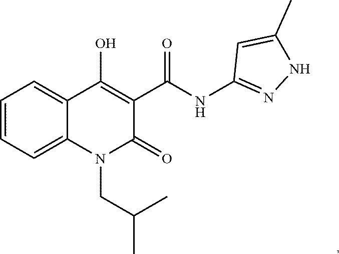 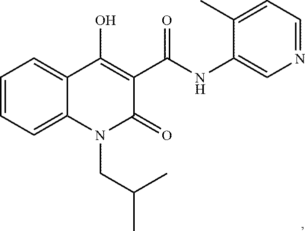 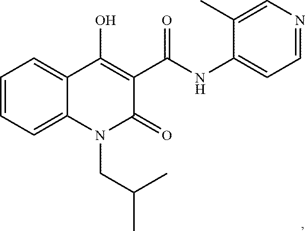 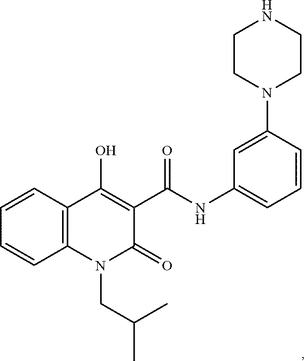 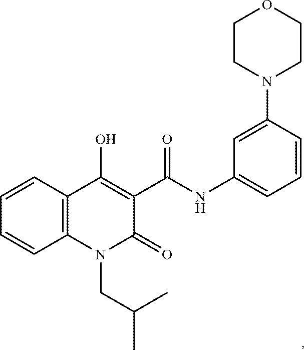 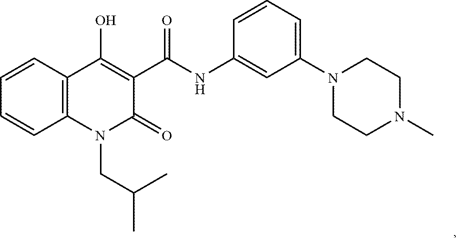 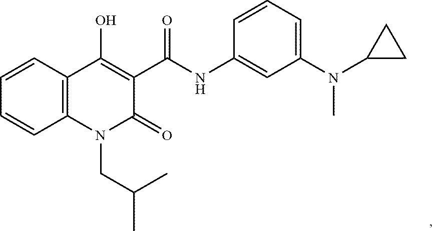 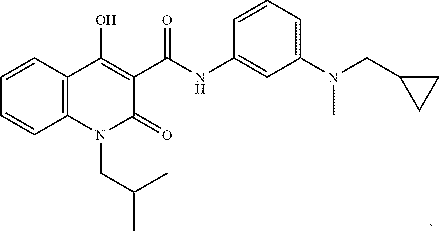 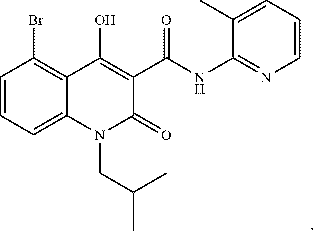 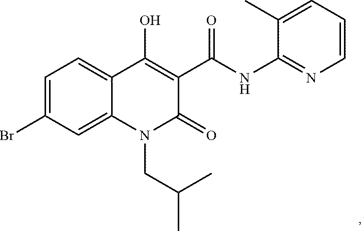 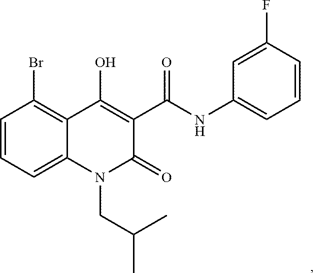 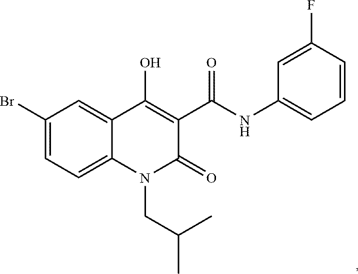 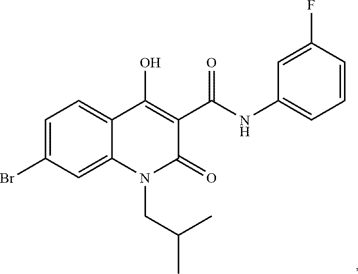 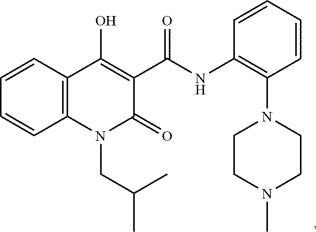 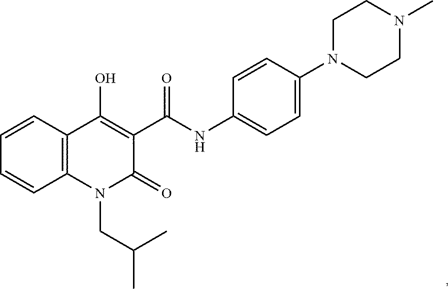 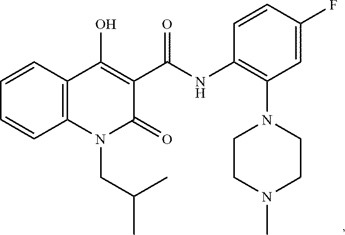 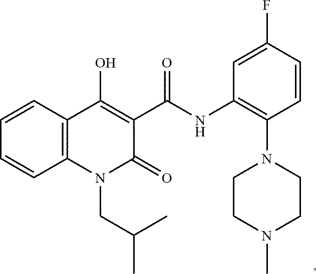 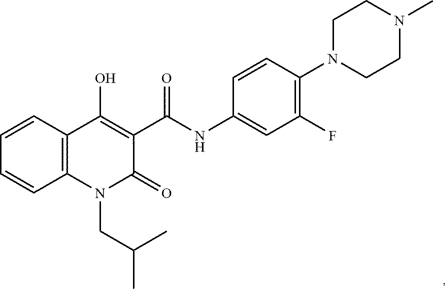 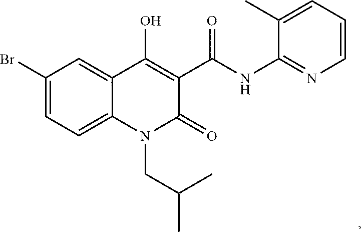 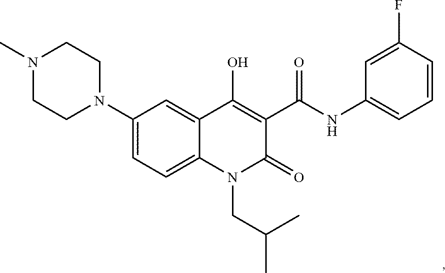 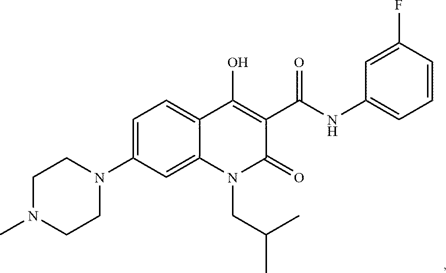 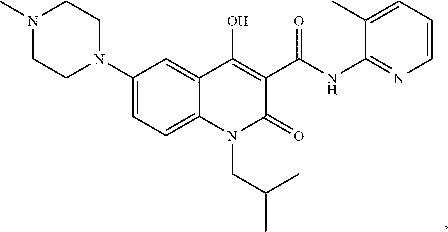 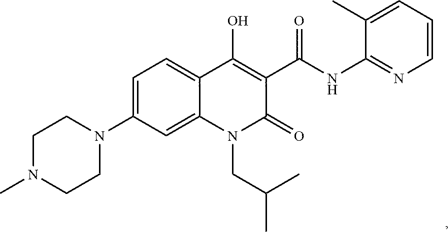 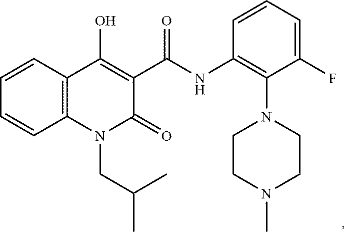 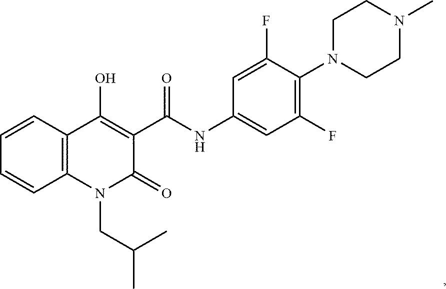 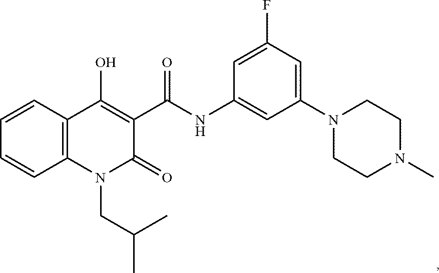 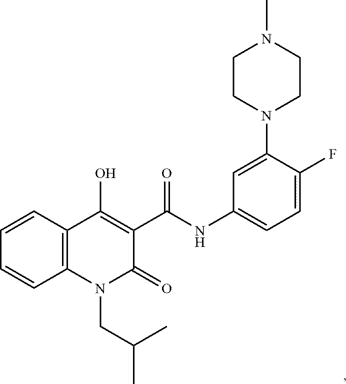 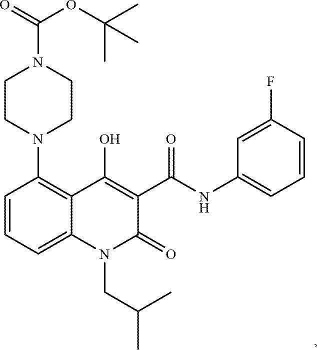 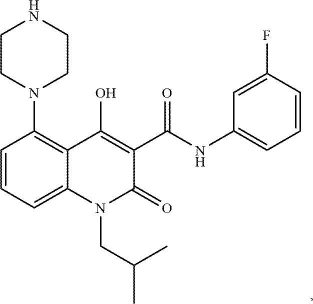 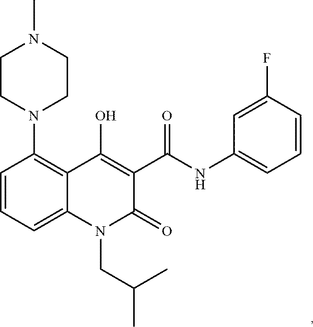 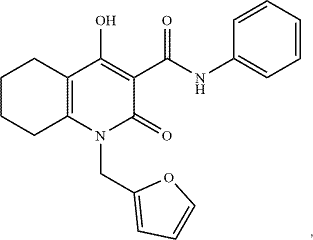 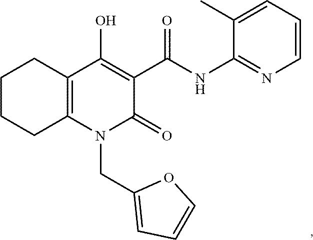 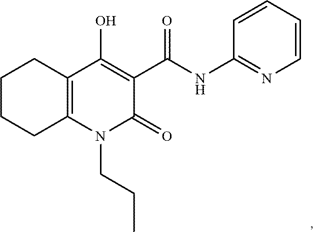 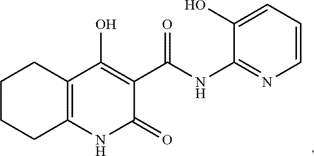 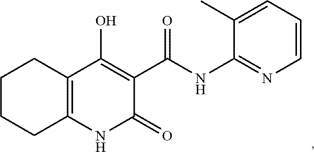 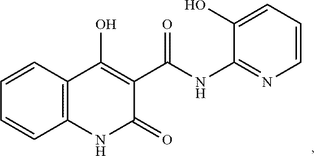 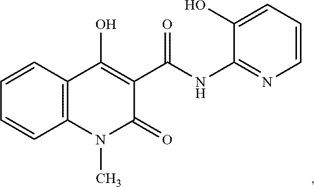 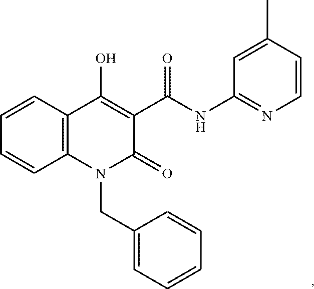 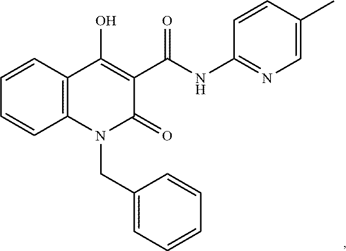 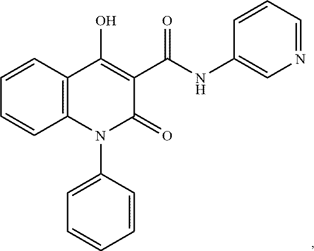 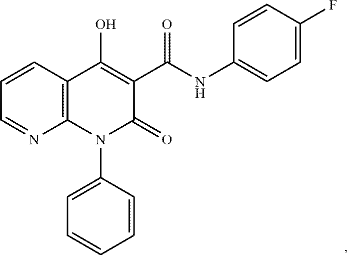 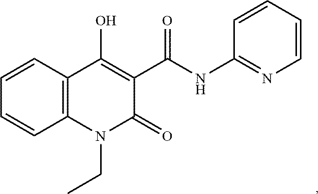 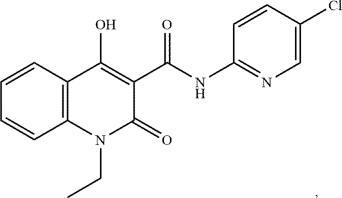 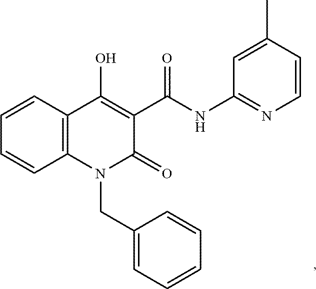 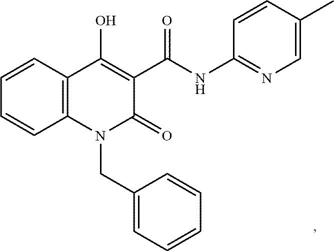 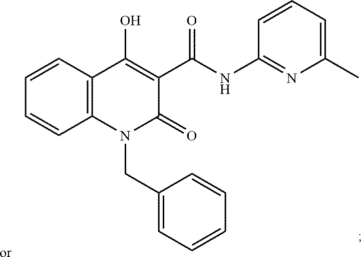
or a pharmaceutically acceptable salt thereof.

33. A pharmaceutical composition comprising the compound of one of claims 1 to 32 and a pharmaceutically acceptable excipient.

34. A method of decreasing the level of Notch protein activity in a subject, said method comprising administering a compound of one of claims 1 to 32 to said subject.

35. The method of claim 34, wherein the compound contacts Notch protein.

36. The method of claim 34, wherein the compound reduces Mastermind binding to Notch.

37. The method of claim 34, wherein the compound reduces CSL binding to Notch.

38. A method of decreasing the level of Notch activity in a cell, said method comprising contacting said cell with a compound of one of claims 1 to 32.

39. The method of claim 38, wherein the compound contacts Notch protein.

40. The method of claim 38, wherein the compound reduces Mastermind binding to Notch.

41. The method of claim 38, wherein the compound reduces CSL binding to Notch.

42. A method of decreasing the level of CSL-Notch-Mastermind complex activity in a subject, said method comprising administering a compound of one of claims 1 to 32 to said subject.

43. The method of claim 42, wherein the compound contacts Notch protein.

44. The method of claim 42, wherein the compound reduces Mastermind binding to Notch.

45. The method of claim 42, wherein the compound reduces CSL binding to Notch.

46. A method of decreasing the level of CSL-Notch-Mastermind complex activity in a cell, said method comprising contacting said cell with a compound of one of claims 1 to 32.

47. The method of claim 46, wherein the compound contacts Notch protein.

48. The method of claim 46, wherein the compound reduces Mastermind binding to Notch.

49. The method of claim 46, wherein the compound reduces CSL binding to Notch.

50. A method of inhibiting cancer growth in a subject in need thereof, said method comprising administering to the subject in need thereof an effective amount of a compound of one of claims 1 to 32.

51. A method of treating a cancer in a subject in need thereof, said method comprising administering to the subject in need thereof an effective amount of a compound of one of claims 1 to 32.

52. The method of claim 51, wherein the cancer is breast cancer, esophageal cancer, leukemia, prostate cancer, colorectal cancer, lung cancer, central nervous system cancer.

53. The method of claim 51, further comprising co-administering an anticancer agent to said subject.

54. A pharmaceutical composition comprising a pharmaceutically acceptable excipient and a compound having the formula: 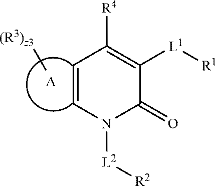
wherein
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or , substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, -N(O)ₘ₁, —NR^{1A}R^{1B}, —C(O)R^{1C}, —C(O)—OR^{1C}, —C(O)NR^{1A}R^{1B}, -OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², —OCHX²₂, —CN, —SOₙ₂R^{2D}, —SOᵥ₂NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 8;
R⁴ is hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D};
R^{1A}, R^{1B}, R^{1C}, R^{1D}, R^{2A}, R^{2B}, R^{2c}, R^{2D}, R^{3A}, R^{3B}, R^{3C}, R^{3D}, R^{4A}, R^{4B}, R^{4D}, R^{L1}, and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴ are independently —F, —Cl, —Br, or —I;
n1, n2, and n3 are independently an integer from 0 to 4; and
m1, m2, m3, v1, v2, and v3 are independently 1 or 2;
or a salt thereof.

55. A method of decreasing the level of Notch protein activity in a subject or decreasing the level of CSL-Notch-Mastermind complex activity in a subject, said method comprising administering to said subject, a compound having the formula: 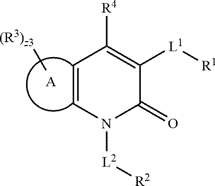
wherein
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or , substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², -OCHX²₂, —CN, —SOₙ₂R^{2D}, —SO_{V2}NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 8;
R⁴ is hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D}.,
R^{1A}, R^{1B}, R^{1C},R^{1D},R^{2A},R^{2B},R^{2c},R^{2D},R^{3A},R^{3B},R^{3C},R^{3D}, R^{4A},R^{4B}, R^{4D},R^{L1} , and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴ are independently —F, —Cl, —Br, or —I;
n1, n2, and n3 are independently an integer from 0 to 4; and
m1, m2, m3, v1, v2, and v3 are independently 1 or 2;
or a salt thereof.

56. The method of claim 55, wherein the compound contacts Notch protein.

57. The method of claim 55, wherein the compound reduces Mastermind binding to Notch.

58. The method of claim 55, wherein the compound reduces CSL binding to Notch.

59. A method of decreasing the level of Notch activity in a cell or decreasing the level of CSL-Notch-Mastermind complex activity in a cell, said method comprising contacting said cell with a compound having the formula: 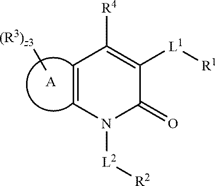
wherein,
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or, substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², —OCHX²₂, —CN, —SOₙ₂R^{2D}, —SO_{V2}NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, —CHX³₂, —CH₂X³, —OCX³₃, —OCH₂X³, -OCHX³₂, —CN, —SOₙ₃R^{3D}, —SOᵥ₃NR^{3A}R^{3B}, —NR^{3C}NR^{3A}R^{3B}, —ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 8;
R⁴ is hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D}.
R^{1A}, R^{1B}, R^{1C}, R^{1D}, R^{2A}, R^{2B}, R^{2c}, R^{2D}, R^{3A}, R^{3B}, R^{3C}, R^{3D}, R^{4A}, R^{4B}, R^{4D}, R^{L1} , and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴ are independently —, —Cl, —Br, or —I;
n1, n2, and n3 are independently an integer from 0 to 4; and
m1, m2, m3, v1, v2, and v3 are independently 1 or 2;
or a salt thereof.

60. The method of claim 59, wherein the compound contacts Notch protein.

61. The method of claim 59, wherein the compound reduces Mastermind binding to Notch.

62. The method of claim 59, wherein the compound reduces CSL binding to Notch.

63. A method of inhibiting cancer growth in a subject in need thereof or treating a cancer in a subject in need thereof, said method comprising administering to the subject in need thereof an effective amount of a compound having the formula: 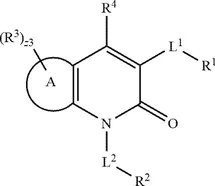
wherein
L¹ is a bond, —N(R^{L1})—, —O—, —S—, —SO₂—, —C(O)—, —C(O)N(R^{L1})—, —N(R^{L1})C(O)—, —N(R^{L1})C(O)NH—, —NHC(O)N(R^{L1})—, —C(O)O—, —OC(O)—, —SO₂N(R^{L1})—, —N(R^{L1})SO₂—, substituted or unsubstituted alkylene, or , substituted or unsubstituted heteroalkylene;
R¹ is hydrogen, halogen, —CX¹₃, —CHX¹₂, —CH₂X¹, —OCX¹₃, —OCH₂X¹, —OCHX¹₂, —CN, —SOₙ₁R^{1D}, —SOᵥ₁NR^{1A}R^{1B}, —NR^{1C}NR^{1A}R^{1B}, —ONR^{1A}R^{1B}, —NHC(O)NR^{1C}NR^{1A}R^{1B}, —NHC(O)NR^{1A}R^{1B}, —N(O)ₘ₁, —NR^{1A}R^{1B}, —C(O)R^{1C}, —C(O)—OR^{1C}, —C(O)NR^{1A}R^{1B}, —OR^{1D}, —NR^{1A}SO₂R^{1D}, —NR^{1A}C(O)R^{1C}, —NR^{1A}C(O)OR^{1C}, —NR^{1A}OR^{1C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
L² is a bond, —N(R^{L2})—, —O—, —S—, —SO₂—, —C(O)—, —C(O)N(R^{L2})—, —N(R^{L2})C(O)—, —N(R^{L2})C(O)NH—, —NHC(O)N(R^{L2})—, —C(O)O—, —OC(O)—, —SO₂N(R^{L2})—, —N(R^{L2})SO₂—, substituted or unsubstituted alkylene, or, substituted or unsubstituted heteroalkylene;
R² is hydrogen, halogen, —CX²₃, —CHX²₂, —CH₂X², —OCX²₃, —OCH₂X², —OCHX²₂, —CN, —SOₙ₂R^{2D}, —SO_{V2}NR^{2A}R^{2B}, —NR^{2C}NR^{2A}R^{2B}, —ONR^{2A}R^{2B}, —NHC(O)NR^{2C}NR^{2A}R^{2B}, —NHC(O)NR^{2A}R^{2B}, —N(O)ₘ₂, —NR^{2A}R^{2B}, —C(O)R^{2C}, —C(O)—OR^{2C}, —C(O)NR^{2A}R^{2B}, —OR^{2D}, —NR^{2A}SO₂R^{2D}, —NR^{2A}C(O)R^{2C}, —NR^{2A}C(O)OR^{2C}, —NR^{2A}OR^{2C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
Ring A is C₅—C₆ cycloalkyl, 5 to 6 membered heterocycloalkyl, phenyl, or 5 to 6 membered heteroaryl;
R³ is independently halogen, oxo, —CX³₃, -CHX³₂, —CH₂X³, —OCX³₃, —OCH₂X³, —OCHX³₂, —CN, —SOₙ₃R^{3D}, —SOᵥ₃NR^{3A}R^{3B}, —NR^{3C}NR^{3A}R^{3B}, -ONR^{3A}R^{3B}, —NHC(O)NR^{3C}NR^{3A}R^{3B}, —NHC(O)NR^{3A}R^{3B}, —N(O)ₘ₃, —NR^{3A}R^{3B}, —C(O)R^{3C}, —C(O)—OR^{3C}, —C(O)NR^{3A}R^{3B}, —OR^{3D}, —NR^{3A}SO₂R^{3D}, —NR^{3A}C(O)R^{3C}, —NR^{3A}C(O)OR^{3C}, —NR^{3A}OR^{3C}, —SF₅, —N₃, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; two R³ substituents may optionally be joined to form a substituted or unsubstituted cycloalkyl, substituted or unsubstituted heterocycloalkyl, substituted or unsubstituted aryl, or substituted or unsubstituted heteroaryl;
z3 is an integer from 0 to 8;
R⁴ is hydrogen, halogen, —CX⁴₃, —CHX⁴₂, —CH₂X⁴, —OCX⁴₃, —OCH₂X⁴, —OCHX⁴₂, —CN, —SR^{4D}, —NR^{4A}R^{4B}, or —OR^{4D}.,
R^{1A}, R^{1B}, R^{1C}, R^{1D}, R^{2A}, R^{2B}, R^{2c}, R^{2D}, R^{3A}, R^{3B}, R^{3C}, R^{3D}, R^{4A}, R^{4B}, R^{4D}, R^{L1} , and R^{L2} are independently hydrogen, —CCl₃, —CBr₃, —CF₃, —CI₃, —CHCl₂, —CHBr₂, —CHF₂, —CHI2, —CH₂Cl, —CH₂Br, —CH₂F, —CH₂I, —CN, —OH, —NH₂, —COOH, —CONH₂, —OCCl₃, —OCF₃, —OCBr₃, —OCI₃, —OCHCl₂, —OCHBr₂, —OCHI2, —OCHF₂, —OCH₂Cl, —OCH₂Br, —OCH₂I, —OCH₂F, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{1A} and R^{1B} substituents bonded to the same nitrogen atom may optionally be joined to form a substituted or unsubstituted heterocycloalkyl or substituted or unsubstituted heteroaryl; R^{2A} and R^{2B} substituents bonded to the same nitrogen atom may optionally be joined to form a substituted or unsubstituted heterocycloalkyl or substituted or unsubstituted heteroaryl; R^{3A} and R^{3B} substituents bonded to the same nitrogen atom may optionally be joined to form a substituted or unsubstituted heterocycloalkyl or substituted or unsubstituted heteroaryl; R^{4A} and R^{4B} substituents bonded to the same nitrogen atom may optionally be joined to form a substituted or unsubstituted heterocycloalkyl or substituted or unsubstituted heteroaryl;
X¹, X², X³, and X⁴ are independently —F, —Cl, —Br, or —I;
n1, n2, and n3 are independently an integer from 0 to 4; and
m1, m2, m3, v1, v2, and v3 are independently 1 or 2;
or a salt thereof.

64. The method of claim 63, wherein the cancer is breast cancer, esophageal cancer, leukemia, prostate cancer, colorectal cancer, lung cancer, central nervous system cancer.

65. The method of claim 63, further comprising co-administering an anticancer agent to said subject in need.
